(19) United States
(12) Patent Application Publication
(10) Pub. No.: US 2016/0184454 A1
(43) Pub. Date: Jun. 30, 2016

(54) RAB7L1 INTERACTS WITH LRRK2 TO MODIFY INTRANEURONAL PROTEIN SORTING AND PARKINSON'S DISEASE RISK

(71) Applicants: Asa ABELIOVICH, New York, NY (US); THE TRUSTEES OF COLUMBIA UNIVERSITY IN THE CITY OF NEW YORK, New York, NY (US)

(72) Inventor: Asa ABELIOVICH, New York, NY (US)

(21) Appl. No.: 14/760,421

(22) PCT Filed: Jan. 13, 2014
(86) PCT No.: PCT/US2014/011226

§ 371 Date: Jul. 10, 2015

Related U.S. Application Data
(60) Provisional application No. 61/751,435, filed on Jan. 11, 2013.

(51) Int. Cl. A61K 48/00

(52) U.S. Cl. CPC A61K 48/005

(57) ABSTRACT

The present invention relates to methods and compositions for treating Parkinson's disease in a subject.

This application claims priority to U.S. Application Ser. No. 61/751,435 filed Jan. 11, 2013, the contents of which is hereby incorporated in its entirety.

### GOVERNMENT INTERESTS

The work described herein was supported in whole, or in part, by National Institute of Health Grant Nos. NS064433 and NS060876. The United States Government has certain rights to the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Parkinson's disease (PD) is a degenerative disorder of the central nervous system. It results from the death of dopamine-containing cells in the substantia nigra, a region of the midbrain; the cause of cell-death is unknown. Early in the course of the disease, the most obvious symptoms are movement-related, including shaking, rigidity, slowness of movement and difficulty with walking and gait. Later, cognitive and behavioral problems may arise, with dementia commonly occurring in the advanced stages of the disease. Other symptoms include sensory, sleep and emotional problems. PD is more common in the elderly with most cases occurring after the age of 50.

Parkinson's disease is diagnosed by a physician exam, and diagnosis is based on the medical history and a neurological examination of the patient. There is no laboratory or molecular test that will clearly identify the disease. Brain scans are sometimes used to rule out disorders that could give rise to similar symptoms. Patients may be given levodopa, or other dopamine affecting agent, and resulting relief of motor impairment tends to confirm diagnosis. The finding of Lewy bodies in the midbrain on autopsy is usually considered proof that the patient suffered from Parkinson's disease. Thus there is need for biomarkers for PD disease or treatment.

### SUMMARY

Recent genome-wide association studies have linked common variants in the human genome to Parkinson's disease (PD) risk. In certain aspects, the invention describes that the consequences of variants at 2 such loci, PARK6 and LRRK2, are highly interrelated, both in terms of their broad impacts on human brain transcriptomes of unaffected carriers, and in terms of their associations with PD risk. Deficiency of the PARK16 locus gene RAB7L1 in primary rodent neurons, or of a RAB7L1 orthologue in Drosophila dopamine neurons, recapitulated degeneration observed with expression of a familial PD mutant form of LRRK2, whereas RAB7L1 overexpression rescued the LRRK2 mutant phenotypes. PD-associated defects in RAB7L1 or LRRK2 led to endolysosomal and Golgi apparatus sorting defects and deficiency of the VPS35 component of the retromer complex. Expression of wild-type VPS35, but not a familial PD-associated mutant form, rescued these defects. Taken together, these studies implicate retromer and lysosomal pathway alterations in PD risk.

In certain aspects the invention provides that genetic variants at PARK16 and LRRK2 interact to modify Parkinson's disease risk.

In certain aspects the invention provides that splicing of the PARK16 locus gene RAB7L1 is modified by genetic variants.

In certain aspects the invention provides that RAB7L1 and LRRK2 coordinately regulate protein sorting through the retromer pathway.

In certain aspects the invention provides that expression of the retromer component VPS35 can suppress LRRK2 mutant pathology.

In certain aspects the invention provides human functional genomics combined with cell and animal model studies, to provide convergent evidence of a critical role for RAB7L1 at the PARK16 locus and of retromer pathway dysfunction in Parkinson's disease etiology.

In certain embodiments, the subject is suspected of or evaluated for having predisposition or risk to sporadic (non-familial) PD.

In certain aspects, the invention provides methods to determine risk or predisposition to develop PD in a subject in need thereof comprising: (a) providing a sample from a subject in need thereof, (b) determining the presence or absence of a genetic variant(s) at PARK16 and LRRK2 locus, and (c) comparing the genetic variant(s) at PARK16 and/or LRRK2 locus from the subject sample to the PARK16 and/or LRRK2 locus variant in a reference sample, wherein the reference sample is associated with a non-PD status. In certain embodiments, the methods further comprise determining whether the gene variant(s) lead to a deficiency of the PARK16 locus gene RAB7L1. In certain embodiments, the deficiency is reduced level of the full-length RAB7L.

The methods can determine the protein level of full-length RAB7L, the full-length RAB7L mRNA levels, or a combination thereof. In other embodiments, the methods can determine the level of VPS35 protein or mRNA, or a combination thereof. The methods can determine the protein level of full-length RAB7L, the full-length RAB7L mRNA levels, the full-length RAB7L mRNA levels, or a combination thereof.

In certain embodiments, the predisposition or risk that is determined is to sporadic (non-familial) PD.

In certain embodiments, the variants are associated with familial PD. In other embodiments, the variants are associated with sporadic PD.

In certain aspects, the invention provides methods to determine risk or predisposition to develop PD in a subject in need thereof comprising: (a) providing a sample from a subject in need thereof, (b) determining the presence or absence of a genetic variant(s) at the PARK16 locus gene RAB7L1, and (c) comparing the genetic variant(s) at PARK16 locus gene RAB7L1 from the subject sample to the PARK16 locus gene RAB7L1 variant(s) in a reference sample, wherein the reference sample is associated with a non-PD status. In certain embodiments, the genetic variant(s) at the PARK16 locus affect the PARK16 locus gene RAB7L1. In certain embodiments, the PARK16 locus gene variant(s) lead to a deficiency of the PARK16 locus gene RAB7L1.

In certain aspects, the invention provides methods to determine a risk or predisposition to develop PD in a subject in need thereof comprising: (a) providing a sample from a subject in need thereof, (b) determining the level of full-length RAB7L, and (c) comparing the level of full-length RAB7L from the subject sample to the full-length RAB7L1 level in a reference sample, wherein the reference sample is associated in non-PD status, and wherein a reduced level of the full-length RAB7L is indicative of an increased risk or predisposition to PD. The methods can determine the protein level of full-length RAB7L, the full-length RAB7L mRNA levels, or a combination thereof. In certain embodiments, the methods comprise determining the full-length RAB7L1 level in a reference sample. In certain embodiments, the methods comprise determining whether the levels the full-length RAB7L1 level in the subject sample are reduced compared to these levels in a reference sample.

In certain aspects, the invention provides methods to determine a risk or predisposition to develop PD in a subject in need thereof comprising: (a) providing a sample from a subject in need thereof, (b) determining the level of VPS35 protein or mRNA, and (c) comparing the level of VPS35 protein or mRNA from the subject sample to the VPS35 protein or mRNA level in a reference sample, wherein the reference sample is associated in non-PD status, and wherein a reduced level of the VPS35 protein or mRNA is indicative of an increased risk or predisposition to PD. In certain embodiments, the methods comprise determining the level VPS35 protein or mRNA in a reference sample. In certain embodiments, the methods comprise determining whether the levels the VPS35 protein or mRNA level in the subject sample are reduced compared to these levels in a reference sample.

In certain embodiments, the methods determine protein or mRNA levels, or a combination thereof.

Certain PARK16 and/or LRRK2 locus variants, including but not limited to variants as described herein, are associated with increased PD risk. In certain embodiments, the PARK16 locus gene is RAB7L1. In certain embodiments, PARK16 and LRRK2 locus variants cooperatively determine PD risk. In certain embodiments, the effect of a risk associated variant at the LRRK2 locus is dependent (or correlated) on the presence of the risk variant at the PARK16 locus, and vice versa. In certain embodiments, there is a genetic interaction between PARK16 and LRRK2 locus variants that affects PD predisposition and risk.

In certain embodiments, the methods further comprise determining whether PD-associated variants or defects in RAB7L1 or LRRK2 lead to endolysosomal, Golgi apparatus sorting defects, deficiency of the VPS35 component of the retromer complex, or any combination thereof.

In certain aspects, the invention provides methods to determine risk or predisposition to develop PD in a subject in need thereof comprising: (a) providing a sample from a subject in need thereof, (b) determining the presence or absence of an endolysosomal, Golgi apparatus sorting defects, deficiency of the VPS35 component of the retromer complex, or any combination thereof, compared a reference sample, wherein the reference sample is associated with a non-PD status.

In certain embodiments, the splicing of the PARK16 locus gene RAB7L1 is modified by genetic variants and is associated with increased risk of PD. In certain embodiments, the PD-associated variants or defects in RAB7L1 or LRRK2 lead to endolysosomal, Golgi apparatus sorting defects, deficiency of the VPS35 component of the retromer complex, or any combination thereof.

In certain embodiments of the methods, the subject is diagnosed with PD and is not administered dopamine affecting agents (i.e. not treated for PD).

In certain embodiments of the methods, the subject is diagnosed by clinical symptoms, imaging of dopamine uptake, or combination thereof.

In certain embodiments, the methods comprise isolating nucleic acids from the subject's biological sample. In the instant methods, the subject's sample is a biological sample, including but not limited to a blood sample, plasma sample, serum, CSF, tissue, cell or any combination thereof. Methods to isolate nucleic acid sequences from biological samples are known in the art. Methods for quantitative determination of amount of nucleic acids in a biological sample are known in the art.

In certain embodiments, the methods comprise quantifying the nucleic acid levels of RAB7L1, VPS35, or any combination thereof, wherein the nucleic acid levels are quantified by RT-qPCR, or any other suitable method. In other embodiments, the methods comprise quantifying the protein levels of RAB7L1. VPS35, or any combination thereof. Methods to determine protein levels in a quantitative manner are known in the art.

In certain embodiments, the sample is a cerebro-spinal fluid (CSF) sample, blood sample, plasma, serum, or any other suitable sample, or any combination thereof.

The invention provides a kit comprising PCR primers to carry out the methods of any of the steps, may also include instructions to carry out steps (a), (b) and (c) of these methods.

A kit comprising at least one nucleic acid, for example but not limited to a primer or a probe, to selectively quantify the levels of RAB7L1. VPS35, or any combination thereof, so as to determine the levels of RAB7L1. VPS3, and instructions to carry out steps (a) and (b) of the method of any of the methods.

In certain aspects, the invention provides methods for treating PD, comprising administering to a subject in need thereof a therapeutic amount of the retromer component VPS35.

An aspect of the invention provides for a method of treating Parkinson's Disease (PD) in a subject. In one embodiment, the method comprises determining the presence or absence of a genetic variant at the PARK16 and LRRK2 loci in a sample from a subject, wherein the presence of a PD-associated genetic variant at both the PARK16 and LRRK2 loci in the subject sample indicates the subject has an increased risk or predisposition to PD; and administering a treatment if the subject has an increased risk or predisposition to PD. In one embodiment, the genetic variant at the PARK16 locus comprises a genetic variant in the RAB7L1 gene. In another embodiment, the genetic variant at the RAB7L1 gene is SNP rs1572931. In a further embodiment, the PD-associated genetic variant at the PARK16 locus comprises a guanine (G) nucleotide at SNP rs1572931. In some embodiments, the PD-associated genetic variant at the PARK16 locus encodes a RAB7L1 mRNA, wherein exon 2 is excluded from the RAB7L1 mRNA sequence. In other embodiments, the the PD-associated genetic variant comprises SEQ ID NO: 5. In further embodiments, the PD-associated genetic variant at the PARK16 locus results in loss of expression of a RAB7L1 protein. In some embodiments, the genetic variant at the LRRK2 locus comprises SNP rs11176052. In other embodiments, the PD-associated genetic variant at the LRRK2 locus encodes the protein of SEQ ID NO: 27 or 28. In yet further embodiments, the PD-associated genetic variant at the LRKK2 locus results in loss of expression of a LRKK2 protein. In one embodiment, the treatment comprises administering to the subject an effective amount of a nucleic acid encoding a protein comprising 90% of SEQ ID NO: 6, SEQ ID NO: 26, SEQ ID NO: 11, SEQ ID NO: 14, or a combination or fragment thereof. In some embodiments, the treatment comprises administering to the subject an effective amount of a protein comprising 90% of SEQ ID NO: 6, SEQ ID NO: 26, SEQ ID NO: 11, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the PARK16 and LRRK2 loci in a sample from a subject, wherein the presence of a PD-associated genetic variant at both the PARK16 and LRRK2 loci in the subject sample indicates the subject has an increased risk or predisposition to PD; and administering a treatment if the subject has an increased risk or predisposition to PD, wherein the treatment comprises administering to the subject an effective amount of a protein comprising 90% of SEQ ID NO: 6. SEQ ID NO: 26, SEQ ID NO: 11. SEQ ID NO: 14, or a combination or fragment thereof. In one embodiment, the genetic variant at the PARK16 locus comprises a genetic variant in the RAB7L1 gene. In another embodiment, the genetic variant at the RAB7L1 gene is SNP rs1572931. In a further embodiment, the PD-associated genetic variant at the PARK16 locus comprises a guanine (G) nucleotide at SNP rs1572931. In some embodiments, the PD-associated genetic variant at the PARK16 locus encodes a RAB7L1 mRNA, wherein exon 2 is excluded from the RAB7L1 mRNA sequence. In other embodiments, the the PD-associated genetic variant comprises SEQ ID NO: 5. In further embodiments, the PD-associated genetic variant at the PARK16 locus results in loss of expression of a RAB7L1 protein. In some embodiments, the genetic variant at the LRRK2 locus comprises SNP rs11176052. In other embodiments, the PD-associated genetic variant at the LRRK2 locus encodes the protein of SEQ ID NO: 27 or 28. In yet further embodiments, the PD-associated genetic variant at the LRKK2 locus results in loss of expression of a LRKK2 protein. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the PARK16 and LRRK2 loci in a sample from a subject, wherein the presence of a PD-associated genetic variant at both the PARK16 and LRRK2 loci in the subject sample indicates the subject has an increased risk or predisposition to PD; and administering a treatment if the subject has an increased risk or predisposition to PD, wherein the treatment comprises administering to the subject an effective amount of a nucleic acid encoding a protein comprising 90% of SEQ ID NO: 6, SEQ ID NO: 26. SEQ ID NO: 11, SEQ ID NO: 14, or a combination or fragment thereof. In one embodiment, the genetic variant at the PARK16 locus comprises a genetic variant in the RAB7L1 gene. In another embodiment, the genetic variant at the RAB7L1 gene is SNP rs1572931. In a further embodiment, the PD-associated genetic variant at the PARK16 locus comprises a guanine (G) nucleotide at SNP rs1572931. In some embodiments, the PD-associated genetic variant at the PARK16 locus encodes a RAB7L1 mRNA, wherein exon 2 is excluded from the RAB7L1 mRNA sequence. In other embodiments, the the PD-associated genetic variant comprises SEQ ID NO: 5. In further embodiments, the PD-associated genetic variant at the PARK16 locus results in loss of expression of a RAB7L1 protein. In some embodiments, the genetic variant at the LRRK2 locus comprises SNP rs11176052. In other embodiments, the PD-associated genetic variant at the LRRK2 locus encodes the protein of SEQ ID NO: 27 or 28. In yet further embodiments, the PD-associated genetic variant at the LRKK2 locus results in loss of expression of a LRKK2 protein. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the LRRK2 locus in a sample from a subject, wherein the presence of a PD-associated genetic variant at the LRRK2 locus in the subject sample indicates the subject has an increased risk or predisposition to PD; and administering a treatment if the subject has an increased risk or predisposition to PD. In some embodiments, the genetic variant at the LRRK2 locus comprises SNP rs11176052. In other embodiments, the PD-associated genetic variant at the LRRK2 locus encodes the protein of SEQ ID NO: 27 or 28. In yet further embodiments, the PD-associated genetic variant at the LRKK2 locus results in loss of expression of a LRKK2 protein. In further embodiments, the treatment comprises administering to the subject an effective amount of a nucleic acid encoding a protein comprising 90% of SEQ ID NO: 6, SEQ ID NO: 26, SEQ ID NO: 14, or a combination or fragment thereof. In some embodiments, the treatment comprises administering to the subject an effective amount of a protein comprising 90% of SEQ ID NO: 6, SEQ ID NO: 26, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the LRRK2 locus in a sample from a subject, wherein the presence of a PD-associated genetic variant at the LRRK2 locus in the subject sample indicates the subject has an increased risk or predisposition to PD; and administering a treatment if the subject has an increased risk or predisposition to PD, wherein the treatment comprises administering to the subject an effective amount of a nucleic acid encoding a protein comprising 90%/o of SEQ ID NO: 6. SEQ ID NO: 26, SEQ ID NO: 14, or a combination or fragment thereof. In some embodiments, the genetic variant at the LRRK2 locus comprises SNP rs11176052. In other embodiments, the PD-associated genetic variant at the LRRK2 locus encodes the protein of SEQ ID NO: 27 or 28. In yet further embodiments, the PD-associated genetic variant at the LRKK2 locus results in loss of expression of a LRKK2 protein. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the LRRK2 locus in a sample from a subject, wherein the presence of a PD-associated genetic variant at the LRRK2 locus in the subject sample indicates the subject has an increased risk or predisposition to PD; and administering a treatment if the subject has an increased risk or predisposition to PD, wherein the treatment comprises administering to the subject an effective amount of a protein comprising 90% of SEQ ID NO: 6, SEQ ID NO: 26, SEQ ID NO: 14, or a combination or fragment thereof. In some embodiments, the genetic variant at the LRRK2 locus comprises SNP rs11176052. In other embodiments, the PD-associated genetic variant at the LRRK2 locus encodes the protein of SEQ ID NO: 27 or 28. In yet further embodiments, the PD-associated genetic variant at the LRKK2 locus results in loss of expression of a LRKK2 protein. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the PARK16 locus in a sample from a subject, wherein the presence of a PD-associated genetic variant at the PARK16 locus in the subject sample indicates the subject has an increased risk or predisposition to PD; and administering a treatment if the subject has an increased risk or predisposition to PD. In one embodiment, the genetic variant at the PARK16 locus comprises a genetic variant in the RAB7L1 gene. In another embodiment, the genetic variant at the RAB7L1 gene is SNP rs1572931. In a further embodiment, the PD-associated genetic variant at the PARK16 locus comprises a guanine (G) nucleotide at SNP rs1572931. In some embodiments, the PD-associated genetic variant at the PARK16 locus encodes a RAB7L1 mRNA, wherein exon 2 is excluded from the RAB7L1 mRNA sequence. In other embodiments, the the PD-associated genetic variant comprises SEQ ID NO: 5. In further embodiments, the PD-associated genetic variant at the PARK16 locus results in loss of expression of a RAB7L1 protein. In one embodiment, the treatment comprises administering to the subject an effective amount of a nucleic acid encoding a protein comprising 90% of SEQ ID NO: 11, SEQ ID NO: 14, or a combination or fragment thereof. In another embodiment, the treatment comprises administering to the subject an effective amount of a protein comprising 90% of SEQ ID NO: 1, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the PARK16 locus in a sample from a subject, wherein the presence of a PD-associated genetic variant at the PARK16 locus in the subject sample indicates the subject has an increased risk or predisposition to PD; and administering a treatment if the subject has an increased risk or predisposition to PD, wherein the treatment comprises administering to the subject an effective amount of a nucleic acid encoding a protein comprising 90% of SEQ ID NO: 11, SEQ ID NO: 14, or a combination or fragment thereof. In one embodiment, the genetic variant at the PARK16 locus comprises a genetic variant in the RAB7L1 gene. In another embodiment, the genetic variant at the RAB7L1 gene is SNP rs1572931. In a further embodiment, the PD-associated genetic variant at the PARK16 locus comprises a guanine (G) nucleotide at SNP rs1572931. In some embodiments, the PD-associated genetic variant at the PARK16 locus encodes a RAB7L1 mRNA, wherein exon 2 is excluded from the RAB7L mRNA sequence. In other embodiments, the the PD-associated genetic variant comprises SEQ ID NO: 5. In further embodiments, the PD-associated genetic variant at the PARK16 locus results in loss of expression of a RAB7L1 protein. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating Parkinson's Disease (PD) in a subject. In one embodiment, the method comprises determining the presence or absence of a genetic variant at the PARK16 locus in a sample from a subject, wherein the presence of a PD-associated genetic variant at the PARK16 locus in the subject sample indicates the subject has an increased risk or predisposition to PD; and administering a treatment if the subject has an increased risk or predisposition to PD, wherein the treatment comprises administering to the subject an effective amount of a protein comprising 90% of SEQ ID NO: 11, SEQ ID NO: 14, or a combination or fragment thereof. In one embodiment, the genetic variant at the PARK16 locus comprises a genetic variant in the RAB7L11 gene. In another embodiment, the genetic variant at the RAB7L1 gene is SNP rs1572931. In a further embodiment, the PD-associated genetic variant at the PARK16 locus comprises a guanine (G) nucleotide at SNP rs1572931. In some embodiments, the PD-associated genetic variant at the PARK6 locus encodes a RAB7L1 mRNA, wherein exon 2 is excluded from the RAB7L1 mRNA sequence. In other embodiments, the the PD-associated genetic variant comprises SEQ ID NO: 5. In further embodiments, the PD-associated genetic variant at the PARK16 locus results in loss of expression of a RAB7L1 protein. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides for a method of treating PD in a subject. In one embodiment, the method comprises measuring the expression levels of a set of genes in a sample from a subject, wherein the set of genes comprises at least one gene selected from the genes listed in Table 2; comparing the expression levels of the set of genes in the subject sample to expression levels of the same set of genes in a reference sample or samples, wherein the reference sample or samples are from an individual who has a PD-associated SNP, and wherein similar expression levels of the set of genes in the subject sample and the set of genes in the reference sample(s) indicates the subject has an increased risk or predisposition to PD; and administering a treatment if the subject has an increased risk or predisposition to PD. In one embodiment, the treatment comprises administering to the subject an effective amount of a nucleic acid encoding a protein comprising 90% of SEQ ID NO: 6, SEQ ID NO: 26, SEQ ID NO: 11, SEQ ID NO: 14, or a combination or fragment thereof. In some embodiments, the treatment comprises administering to the subject an effective amount of a protein comprising 90% of SEQ ID NO: 6, SEQ ID NO: 26, SEQ ID NO: 11, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof.

An aspect of the invention provides a method of treating PD in a subject. In one embodiment, the method comprises determining a level of full-length RAB7L1 in a sample from a subject; comparing the level of full-length RAB7L1 from the subject sample to a full-length RAB7L1 level in a reference sample, wherein the reference sample is associated with a non-PD status, and wherein a reduced level of the full-length RAB7L in the subject sample indicates the subject has an increased risk or predisposition to PD; and administering a treatment if the subject has an increased risk or predisposition to PD. In one embodiment, the method comprises the level of full-length RAB7L is protein level of full-length RAB7L, or mRNA levels of the full-length RAB7L, or a combination thereof. In one embodiment, the treatment comprises administering to the subject an effective amount of a nucleic acid encoding a protein comprising 90% of SEQ ID NO: 6, SEQ ID NO: 26, SEQ ID NO: 11, SEQ ID NO: 14, or a combination or fragment thereof. In some embodiments, the treatment comprises administering to the subject an effective amount of a protein comprising 90% of SEQ ID NO: 6, SEQ ID NO: 26, SEQ ID NO: 1, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof. In another embodiment, the method further comprises a step of sequencing nucleic acids isolated from the subject's sample to determine the presence or absence of a PD-risk associated SNP, wherein the presence of a PD-risk associated SNP is further indicative that the subject is at risk of developing PD or is suffering from PD.

An aspect of the invention provides for a method of treating Parkinson's Disease (PD) in a subject. In one embodiment, the method comprises determining a level of isoform 3 of RAB7L1 in a sample from a subject; comparing the level of isoform 3 of RAB7L1 from the subject sample to an isoform 3 of RAB7L1 level in a reference sample, wherein the reference sample is associated in non-PD status, and wherein an increased level of isoform 3 of RAB7L1 in the subject sample indicates the subject has an increased risk or predisposition to PD; and administering a treatment if the subject has an increased risk or predisposition to PD. In another embodiment, the level of isoform 3 of RAB7L1 is a protein level. In a further embodiment, the method further comprises determining the level of transcript variant 4, 5, or a combination thereof of RAB7L1. In one embodiment, the treatment comprises administering to the subject an effective amount of a nucleic acid encoding a protein comprising 90% of SEQ ID NO: 6, SEQ ID NO: 26, SEQ ID NO: 11. SEQ ID NO: 14, or a combination or fragment thereof. In some embodiments, the treatment comprises administering to the subject an effective amount of a protein comprising 90% of SEQ ID NO: 6, SEQ ID NO: 26, SEQ ID NO: 11, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof. In another embodiment, the method further comprises a step of sequencing nucleic acids isolated from the subject's sample to determine the presence or absence of a PD-risk associated SNP, wherein the presence of a PD-risk associated SNP is further indicative that the subject is at risk of developing PD or is suffering from PD.

An aspect of the invention provides a method of treating Parkinson's Disease (PD) in a subject. In one embodiment, the method comprises determining a level of transcript variant 4, 5, or a combination thereof of RAB7L1 in a sample from a subject; comparing the level of transcript variant 4, 5, or a combination thereof of RAB7L1 from the subject sample to a transcript variant 4, 5, or a combination thereof of RAB7L1 level in a reference sample, wherein the reference sample is associated in non-PD status, and wherein an increased level of transcript variant 4, 5, or a combination thereof of RAB7L1 in the subject sample indicates the subject has an increased risk or predisposition to PD; and administering a treatment if the subject has an increased risk or predisposition to PD. In another embodiment, the level of transcript variant 4, 5 or a combination thereof of RAB7L1 is a mRNA level. In a further embodiment, the method further comprises determining the level of isoform 3 of RAB7L1. In one embodiment, the treatment comprises administering to the subject an effective amount of a nucleic acid encoding a protein comprising 90% of SEQ ID NO: 6, SEQ ID NO: 26. SEQ ID NO: 11, SEQ ID NO: 14, or a combination or fragment thereof. In some embodiments, the treatment comprises administering to the subject an effective amount of a protein comprising 90% of SEQ ID NO: 6, SEQ ID NO: 26, SEQ ID NO: 11, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof. In another embodiment, the method further comprises a step of sequencing nucleic acids isolated from the subject's sample to determine the presence or absence of a PD-risk associated SNP, wherein the presence of a PD-risk associated SNP is further indicative that the subject is at risk of developing PD or is suffering from PD.

An aspect of the invention provides a method of treating Parkinson's Disease (PD) in a subject. In one embodiment, the method comprises determining a level of retromer components in a sample from a subject; comparing the level of retromer components from the subject sample to a retromer component level in a reference sample, wherein the reference sample is associated with a non-PD status, and wherein a reduced level of the retromer components in the subject sample indicates the subject has an increased risk or predisposition to PD; and administering a treatment if the subject has an increased risk or predisposition to PD. In another embodiment, the level of retromer component is protein level of retromer component, or mRNA levels of retromer component, or a combination thereof. In a further embodiment, the retromer component is VPS35, VPS29, VPS26 or a combination thereof. In some embodiments, the level of VPS35. VPS29, or VPS26 is protein level of VPS35, VPS29, or VPS26, or mRNA levels of VPS35. VPS29, or VPS26, or a combination thereof. In one embodiment, the treatment comprises administering to the subject an effective amount of a nucleic acid encoding a protein comprising 90% of SEQ ID NO: 6. SEQ ID NO: 26, SEQ ID NO: 11. SEQ ID NO: 14, or a combination or fragment thereof. In some embodiments, the treatment comprises administering to the subject an effective amount of a protein comprising 90% of SEQ ID NO: 6, SEQ ID NO: 26, SEQ ID NO: 11, SEQ ID NO: 14, or a combination or fragment thereof. In further embodiments, the PD disease status is determined by any suitable method, including but not limited to a physical examination of the subject, a neurological examination of the subject, a brain scan, or a combination thereof. In another embodiment, the subject is not diagnosed with PD. In yet other embodiments, the method further comprises a physical examination of the subject, a neurological examination of the subject, a brain scan, or a combination thereof. In some embodiments, the subject is suspected of having PD or is at risk of developing PD based on the presence of any one of parkinsonism symptoms. In further embodiments, the sample is a CSF sample, blood sample, plasma sample, serum sample or any combination thereof. In another embodiment, the method further comprises a step of sequencing nucleic acids isolated from the subject's sample to determine the presence or absence of a PD-risk associated SNP, wherein the presence of a PD-risk associated SNP is further indicative that the subject is at risk of developing PD or is suffering from PD.

An aspect of the invention provides for a composition for evaluating the existence of, or predisposition to, PD in a subject, said composition comprising polynucleotides or oligonucleotides, wherein each polynucleotide or oligonucleotide hybridizes to a gene, gene fragment, or gene transcript of at least two different markers in a subject sample, wherein the markers comprise LRRK2, RAB7L1 and VPS35.

An aspect of the invention provides for a composition for evaluating the existence of, or predisposition to, PD in a subject, said composition comprising polynucleotides or oligonucleotides, wherein each polynucleotide or oligonucleotide hybridizes to a gene, gene fragment, or gene transcript of a different marker in a subject sample, each marker being one of the genes listed in Table 2. In one embodiment, the composition comprises a microarray, a microfluidics card, a chip, or a chamber.

An aspect of the invention provides a kit for determining the levels of RAB7L1, LRRK2, VPS35, or a combination thereof, the kit comprising at least one oligonucleotide or polynucleotide to selectively quantify the levels of RAB7L1. LRRK2. VPS35, or a combination thereof. In one embodiment, the oligonucleotide or polynucleotide comprises SEQ ID NO: 15, 16, 17, or 18.

An aspect of the invention provides for a diagnostic kit for determining whether a sample from a subject exhibits a presence or absence of a PD-associated genetic variant, the kit comprising at least one oligonucleotide or polynucleotide for sequencing nucleic acids isolated from the subject's sample to determine the presence or absence of a PD-risk associated SNP, wherein the presence of a PD-risk associated SNP is further indicative that the subject is at risk of developing PD or is suffering from PD. In one embodiment, the oligonucleotide or polynucleotide comprises SEQ ID NO: 24, or 25.

An aspect of the invention provides for a diagnostic kit comprising the microarray, microfluidics card, chip, or chamber described herein.

An aspect of the invention provides for a synthetic nucleic acid comprising SEQ ID NO: 15, 16, 17, 18, 19, 24, or 25.

### BRIEF DESCRIPTION OF THE FIGURES

To conform to the requirements for PCT patent applications, many of the figures presented herein are black and white representations of images originally created in color, such as many of those figures based on immunofluorescence microscopy. In the below descriptions and the examples, this colored staining is described in terms of its appearance in black and white. For example, GFP staining which appeared green in the original appears as a grey stain when presented in black and white. The original color versions of FIGS. 1-13 can be viewed in MacLeod et al., (2013) Neuron. 77(3):425-39 (including the accompanying Supplementary Information available in the on-line version of the manuscript available on the Neuron web site). For the purposes of the PCT, the contents of MacLeod et al., (2013) Neuron. 77(3):425-39, including the accompanying “Supplementary Information.” are herein incorporated by reference.

FIGS. 1A-1B. LRRK2 and PARK16 PD risk-associated variants function in a common genetic pathway. (1A) PD risk-associated variants exert functional effects in the CNS of unaffected individuals that is assessed in terms of a global transcriptome impact. Similar to the one observed in PD affected brain, it may reflect a pre-disease prodromal state. (1B) Schematic of GPI analysis. Without being bound by theory, PD risk-associated genotypes at 2 independent loci (upper panels) differentially alter the function of a nearby gene (red star in middle panel). This secondarily impacts the brain transcriptome (lower panels), with significant overlap for different PD-risk genotype shows.

FIGS. 1C-1D. LRRK2 and PARK16 PD risk-associated variants function in a common genetic pathway. (1C) Hierarchical clustering dendrogram shows that the gene expression signatures across 7 PD-associated variant GPIs (“Risk GPI”; in unaffected cerebral cortex Broadmann Area 9 [BA9]) are most similar to the signatures seen in PD brain (BA9 or substantia nigra; SN; in red (e.g., PD/S.N. and PD/Cx.) rather than in other CNS diseases such as Alzheimer's disease, Huntington's disease, Bipolar Disorder or Schizophrenia. 352 gene transcript expression patterns—corresponding to the intersection of the PD risk variants GPIs (FIG. 8A-C)—were interrogated. Clustering was performed using Pearson's distance with complete linkage (see Methods). (1D) Genetic interaction between PARK16 and LRRK2 alleles revealed by GPI analysis in 185 unaffected brain samples (GEO GSE15222 “Initial”) and in an independent cohort of 143 unaffected brain samples (GEO GSE15745. “Replication”), as established by the interaction factor between pairs of GPIs as indicated, in a linear regression model (see Methods). The p-value (“p”) associated with the interaction term as well as its orientation (“Dir.”) are presented. Results combined across both cohorts presented (“Combined”) with the resulting Z-scores and p-values for interaction.

FIGS. 1E-1F. LRRK2 and PARK16 PD risk-associated variants function in a common genetic pathway. (1E) The PARK16 genotype modifies LRRK2 associated risk in sporadic PD. A table presents the odds ratios for PD at the LRRK2 locus as a function of the PARK16 genotype in 4 independent GWAS cohorts: 1 of Ashkenazi Jews (“AJ”, n=417) and 3 of Caucasians (“NGRC”, n=4008; “NINDS”, n=537; “MAYO”, n=886). (1F) Manhattan plot of the Chr1 region reported as a modifier of age of disease onset in familial PD with LRRK2 mutation (Latourelle et al., 2011). Epistasis was evaluated for 74 SNPs in 4 independent sporadic PD GWAS datasets. X-axis represents chromosomal location, Y-axis represents −log 10 of the combined p-value for epistasis of each SNP with the PD risk SNP rsl 1176052 at the LRRK2 locus. The PARK16 locus PD-associated SNP rs823114 (arrow) exhibited the most significant association (p=4.6 E-6; red line (shown as grey in black and white image) represents the significance threshold after correction for multiple testing).

FIG. 2A shows schematics of the PARK16 locus on chromosome 1.

FIG. 2B. Overexpression of the PARK16 locus gene RAB7L1 specifically rescues a LRRK2 mutant phenotype. RAB7L1 modifies a LRRK2-associated neurite process length phenotype. Rat primary cortical neuron cultures transfected with a vector expressing G2019S mutant LRRK2 displayed reduced total neurite length relative to vector alone (cells are co-transfected with GFP for visualization by fluorescence microscopy). (i) (ii) Co-transfection of a wild-type or constitutively active (CA) RAB7L1 expression vector (1 μg/well) along with LRRK2 G2019S (0.5 μg/well) significantly rescued neurite length; other PARK16 genes—NUCKS1, SLC45A3, PM20D1, and SLC41A1—failed to rescue. CA or inactive (IN) RAB vectors were also tested as indicated (left panel; GFP-tagged at the N-terminus; 1 μg/well). (iii) Knock-down of RAB7L1 by shRNA vector transfection led to a similar decrease in neurite length as with LRRK2 G2019S expression. n=20 neurons in 4 independent cultures per group. Mean total neurite lengths are displayed; error bars represent SEM, *: p<0.05, **: p<0.01, ***: p<0.001 for ANOVA followed by Tukey's HSD post hoc analysis.

FIGS. 3A-3C. Evidence of a RAB7L1-LRRK2 complex. (3A) Immunoprecipitation (IP) analysis of RAB7L1 from lysates of HEK293T cells transfected with plasmids encoding a GFP-RAB7L1 fusion protein (or vector alone) and a 3×flag (3FL) epitope-tagged LRRK2 construct (either wild type [WT], G2019S [GS], K1906M [KM], or empty vector). IP with an anti-flag antibody was followed with immunoblot (IB) analysis with an anti-GFP or an anti-LRRK2 antibody as indicated. Arrowheads indicate the expected protein sizes. (3B) Co-immunoprecipitation of LRRK2 with RAB7L1 from lysates of HEK293T cells transfected with a plasmid encoding a 3×flag LRRK2 construct and a plasmid encoding a GFP-RAB7L1 fusion protein (either WT, CA, IN, or GFP only). (3C) Immunoprecipitation using an anti-LRRK2 antibody from whole brain lysates of non-transgenic (NT), LRRK2 wild type transgenic (WT), LRRK2 R1441C (RC) transgenic, or LRRK2 knockout (−/−) mice. IB was subsequently performed for RAB7L1, RAB11 and LRRK2.

FIG. 3D. Evidence of a RAB7L1-LRRK2 complex. (3D) Subcellular co-localization of RAB7L1 and LRRK2. Human neuroblastoma SH-SY5Y cells were transfected with GFP-tagged RAB7L1 vectors (in green (shown as light grey in black and white image)); either WT, CA, or IN forms, as well as a RAB7L1 construct lacking exon 2 and 3 and corresponding to an alternatively spliced RAB7L1 transcript, “AT”) and a 3×flag-tagged LRRK2 vector (in red, left panel (shown as grey in black and white image)). Subcellular localization was determined by immunostaining with a marker for the Golgi apparatus (Golph4; in blue (shown as dark grey in black and white image)). The CA form leads to a reduced localization to the Golgi apparatus. Co-localization is evaluated by quantifying the fraction of RAB7L1/Golph4, RAB7L1/LRRK2 and LRRK2/Golph4 staining overlap (Upper, lower and middle right panels respectively). Results represent mean±SEM (n=15 per group).

FIG. 4A. RAB7L1 rescues lethality and dopamine neuron loss in a Drosophila model of LRRK2 G2019S neurodegeneration. Modifier screen for suppressors of an early adult lethality phenotype seen with expression of LRRK2 G2019S selectively in tyrosine hydroxylase (TH)-positive dopamine neurons. Left, a panel of 16 Drosophila RAB transgenes was screened (of 31 total; see Table 3). Adult survival (days post-eclosion) curves are presented for individual strains harboring different RABs along with the LRRK2 G2019S transgene. Non-transgenic survival curve is shown for comparison. n>25 for all conditions. Right, Adult survival (days post-eclosion) of Drosophila is presented in the context of transgenic expression of LRRK2 (WT or G2019S), with or without RAB7L1 (WT, CA or IN), using a tyrosine hydroxylase promoter GAL4 driver for dopaminergic neuron expression. Non-transgenic survival is also shown for comparison. n>25 for all conditions.

FIG. 4B. RAB7L1 rescues lethality and dopamine neuron loss in a Drosophila model of LRRK2 G2019S neurodegeneration. (left) Confocal microscopy of mushroom bodies of the CNS from transgenic Drosophila as in (FIG. 4A), with dopaminergic neuron nuclei visualized using an additional marker transgene, a nuclear localization sequence (NLS)-GFP fusion, also driven by TH-Gal4. (Right) Quantitation of surviving dopaminergic neurons in the PPM1 and PPL1 clusters of Drosophila CNS mushroom bodies. Means are displayed; error bars represent SEM; ***:p<0.001 by ANOVA followed by Tukey's HSD post hoc analysis for (4A) and (4B).

FIGS. 5A-5D. PARK16 PD risk-associated variants modify RAB7L1 splicing and protein accumulation. (5A) Exonic structure of the human RAB7L1 gene. (5B) Analysis of RAB7L1 alternative splicing in human cortical brain samples. The rs1572931 allele G, linked to the PD high-risk haplotype (R), is associated with an increase in the fraction of RAB7L1 transcripts that lack the exon 2 to exon 3 junction region (termed exon 2 skipping; presented relative to the extent of exon 2 skipping seen in carriers of the rs1572931 protective allele A; quantified by qrtPCR using primers as depicted by red and black arrows in (5A) detecting respectively the amount of total and unskipped RAB7L1 mRNA; n=15 and 57 for P and R respectively; details in Table 6). (SC) (i) Schematic of predicted splice site enhancer and silencer motifs upstream of RAB7L1 exon2 and affected by rs1572931 variants G (associated with increased PD risk, “R”) and A (protective, “P”, associated with decreased PD risk). (ii) Structure of a minigene construct to assess the effect of rs1572931 variants on RAB7L1 exon2 inclusion in vitro. Green arrows indicate the position of the primers used to assess exon 2 inclusion. (5D) Impact of PARK16 variants on splicing in vitro. The rs1572931 allele G (associated with increased PD risk, R; relative to the allele A associated with decreased PD risk, P) leads to a relative decrease in RAB7L1 exon 2 inclusion in transfected human SH-SY5Y cells as assess by PCR gel quantification (pictures in FIGS. 12D-E; n=6/group).

FIG. 5E is a bar graph showing the impact of rs1572931 on RAB7L1protein level in human cortical brain samples. rs1572931 allele G is associated with a decrease in RAB7L1 protein level in non-PD post-mortem human cortical brain samples, as assessed by Western Blot from individuals homozygous for the risk allele (R, n=25) and from carriers of the protective allele (P, n=13). Mean levels are displayed; errors bars are SEM; *: p<0.05, **: p<0.01, ***: p<0.001 by two-tailed t-test (5B, 5D) or by linear regression analysis (5E).

FIGS. 6A-6C. RAB7L1 and LRRK2 modulate lysosome and Golgi apparatus sorting in a retromer-dependent manner. (6A-6C) Analysis of MPR sorting in primary rat neuron cultures transfected with vectors encoding LRRK2 G2019S mutant (GS), RAB7L1, VPS35, or VPS35 D620N: or with shRNA plasmids for VPS35, RAB7L1 or vector only, co-transfected with GFP vector for visualization and immunostained for MPR as well as either the Golgi marker Golph4 (6A, upper panel), the lysosome marker Lamp2 (6B, upper panel) or with the early endosome marker EEA1 (6B, upper panel). MPR colocalization with either the Golph4 or LAMP2 marker was reduced with G2019S LRRK2, VPS35 D620N, or knockdown of either RAB711 or VPS35 (6A, lower panel; 6B, lower panel). These manipulations also increased total LAMP2 staining (but not Golph4 staining). Scale bar represents 10 um. Quantifications of the MPR co-localization and of total organelle marker analyses are presented in the lower panels. Error bars represent SEM. n>10 cells in 3 independent wells per group. *: p<0.05, **: p<0.01, ***: p<0.001 for comparisons with “vector” group, ++: p<0.01, +++: p<0.001 for comparisons with “LRRK2 G2019S” group by ANOVA followed by Tukey's HSD post hoc analysis.

FIG. 6D is a schematic showing cell sorting phenotype associated with defects in the LRRK2-Rab7L1 pathway or knockdown of the VPS35 retromer component. MPR accumulation at Golph4-positive structures (trans-golgi network [TGN]) and at LAMP2-positive structures (lysosomes and late endosomes [LE]) is reduced, and lysosomes appear swollen.

FIG. 7A. Evidence of retromer Insufficiency in the context of LRRK2-RAB7L1 pathway defects. Transfection of rat primary cortical neuron cultures with a wild-type (WT) VPS35 expression vector rescued the reduced neurite length phenotype associated with LRRK2 G2019S (GS) mutant expression or with Rab7L1 (R7L1) knockdown. Overexpression of a familial PD mutant VPS35 D620N vector leads to reduced neurite length relative to vector alone. Knockdown of VPS35 by shRNA leads to similarly reduced neurite length relative to vector alone, which is not rescued by Rab7L1 overexpression (n=20 neurons in 4 cultures per group).

FIG. 7B. Evidence of retromer insufficiency in the context of LRRK2-RAB7L1 pathway defects. (Left) Confocal microscopy of mushroom bodies of the CNS from transgenic Drosophila with dopaminergic neuron nuclei visualized using a TH-Gal4-driven nuclear localization sequence (NLS)-GFP fusion. (Right) Quantitation of surviving dopaminergic neurons in the PPM1 and PPL1 clusters of Drosophila CNS mushroom bodies.

FIG. 7C. Evidence of retromer insufficiency in the context of LRRK2-RAB7L1 pathway defects. Relative quantification by western blot of VPS35 (left) or VPS29 (right) protein levels in lysates from mouse neuroblastoma (N2a) cells transfected with vectors encoding VPS35 WT, PS35 shRNA, VPS35 D620N, LRRK2 WT, LRRK2 G2019S (GS), RAB7L1, RAB7L1 shRNA, or vector control (N=3/group).

FIG. 7D. Evidence of retromer insufficiency in the context of LRRK2-RAB7L1 pathway defects. LRRK2 impacts the levels of retromer components in mouse brain. Relative quantification by Western blotting of VPS35 (left), VPS29 (middle) and VPS26 (right) levels in brain tissue samples from non-transgenic (“nTg”), LRRK2 wild-type (“LRRK2-WT”) and LRRK2 R1441C mutant (“LRRK2-RC”) BAC transgenic mice (N=3/group).

FIG. 7E. Evidence of retromer insufficiency in the context of LRRK2-RAB7L1 pathway defects. Immunoprecipitation (IP) analysis of RAB7L1 from lysates of SH-SY5Y cells transfected with plasmids encoding a GFP-VPS35 fusion protein with VPS35 wild-type sequence (“WT”) or the familial PD mutant D620M (“D620N) or vector alone, along with a LRRK2 construct or an empty vector. IP with an anti-GFP antibody was followed with Western immunoblot analysis with an anti-LRRK2 or anti-GFP antibody as indicated. Arrowheads indicate the expected protein sizes.

FIG. 7F. Evidence of retromer insufficiency in the context of LRRK2-RAB7L1 pathway defects. IP using an anti-LRRK2 antibody from whole brain lysates of non-transgenic (NT), LRRK2 wild type transgenic (WT), LRRK2 R1441C (RC) transgenic, or LRRK2 knockout (−/−) mice as in FIG. 3D. Immunoblot was subsequently performed for VPS35 and β-Actin.

FIG. 7G. Evidence of retromer insufficiency in the context of LRRK2−RAB7L1 pathway defects. VPS3S mRNA levels in substantia nigra tissue as determined by meta-analysis of 5 gene expression microarray datasets (Table 5) in 63 unaffected individuals and 81 PD patients samples (left panel) and in laser-microdissected substantia nigra dopaminergic neurons from 8 unaffected individuals and 10 PD patients samples (right panel, GEO GSE20141). Expression levels are normalized to mean of the unaffected group.

FIGS. 7H-7I. Evidence of retromer insufficiency in the context of LRRK2-RAB7L1 pathway defects. (H) VPS35 mRNA in cerebral cortex tissue as determined by high-throughput sequencing of the 3′UTR ends of polyadenylated mRNA transcripts on a cohort of 17 unaffected and 17 PD cerebral cortical tissue samples. Levels are expressed as reads per million (rpm). (I) VPS35 mRNA levels in Globus Pallidus Interna (Gpi) samples (n=10/group GEO GSE20146). Expression levels are normalized to mean of the unaffected group. For all graphs means are displayed, error bars represent SEM; p<0.05(*) p<0.01(**) for ANOVA followed by Tukey's HSD post hoc analysis (7A, 7B, 7C) or by two-tailed t-test (7G, 7H).

FIG. 8A is a graph depicting GPIs of SNPs at 7 PD-associated loci that show a high degree of overlap. Overlap was quantified in terms of number of gene expression profiles that were impacted in the same direction by 7 disease risk-associated haplotypes (at the SNCA, MAPT, LRRK2, PARK16, HLA-DRA, STK39 and LAMP3 loci). 352 were impacted in the same orientation (up or down, of 8560 queried) by all 7 SNPs. Such overlap is highly significant, as shown by analysis of randomly chosen sets of SNP variants. Analysis of 25000 randomly chosen sets of 7 SNPs is shown for comparison, with average of 19.5 overlapping genes and count distribution as shown. p=1E-5 using Wald statistics (see Methods).

FIG. 8B is a histogram of the resampling result for the estimation of the significance between the PD-risk GPI and the expression profile characteristic of PD in prefrontal cortex.

FIG. 8C is a schematic that shows the correlation pattern for each of the genes belonging to the PD-risk intersection GPI with a FDR<5% and that also shows a significant difference (p<0.05, two tailed t test) in their expression levels in either BA9 or SN for a PD vs unaffected comparison.

FIGS. 8D-8E shows Gene Ontology categories enriched in genes whose expression levels are positively (red (first 5 rows)) or negatively (blue (last 5 rows)) associated with the PD risk-associated allelic load for all PD loci (8D) and specifically for the LRRK2 and PARK16 loci (8E). Analysis were conducted using DAVID.

FIG. 9A is a bar graph depicting total neurite length of rat primary cortical neurons transfected with vector or LRRK2 WT or LRRK2 R1441C (0.5 ug per well) and RAB7L1 expressing vector or empty vector (1 ug per well) as indicated. Means are represented, errors bars are SEM. N=20 per group.

FIGS. 9B-9C. Rab7L1 knockdown efficiency measured by Western blot quantitation in 3 independent vector or Rab7L1 shRNAtransfected cultures (9B). Graph shows relative band intensity+/−SEM * p<0.05 by two-tailed t-test. Validation of PARK16 locus genes overexpression vectors by Western Blot (9C). Lysates from cells tranfected with the PARK16 gene indicated (+) or control vector (−) probed by immunoblot using corresponding antibodies that recognize both endogenous and exogenous PARK16 gene expression. Constructs were transfected in cell lines of matching species (human SH-SY5Y for RAB7L1 and NUCKS1; mouse N2a for SLC41A1 and SLC45A3). 30 ug protein was loaded per lane. Beta-actin loading control is shown below.

FIG. 10A is a photographic image of Rab7L1 and LRRK2 immunohistochemistry of substantia nigra section from non-transgenic, LRRK2 WT, and LRRK2 R1441C transgenic mice. Tyrosine hydroxylase (TH) staining (in green (shown as light grey in black and white image)) marks dopaminergic neurons.

FIG. 10B is a photographic image of an immunoblot analysis of N2a cells transiently expressing wildtype or mutant forms RAB7L1 as indicated. 30 μg of cell lysate was loaded in each lane. Arrrowheads indicate RAB7L1 as detected by an anti-GFP antibody; the DN form leads to a smaller product as expected.

FIG. 11 is a bar graph showing a negative geotaxis analysis of lrrk mutant Drosophila. Lrrk mutant (−/−) Drosophila defective negative geotaxis can be rescued by pan-neuronal expression of human LRRK2 WT or G2019S transgenes. Bars represent Mean times to climb 10 cm upward in a cylinder are represented. Error bars are SEM. ***: p<0.001, for comparison with the non-transgenic group by ANOVA (df=3, F=13.14) followed by Tukey HSD post hoc analysis.

FIG. 12A is a schematic showing the exons/introns structure of the RAB7L1 gene and its different known isoform products.

FIG. 12B is a schematic showing the exons/introns structure of an artificial RAB7L1 minigene and expected isoform products.

FIG. 12C is a schematic showing the RAB7L1 protein functional domains as predicted by CD-search, in parallel with the exonic structure of the CDS. The dashed red line indicates the alternative start site of the CDS in the event of exon 2 exclusion.

FIG. 12D is a photographic image of a gel showing rtPCR products to assess the splice of a RAB7L1 exon 2 reporter in SH-SY5Y cells transfected with a minigene bearing one of the two rs1572931 alleles. The numbered arrows correspond to the different isoforms expected from the minigene as depicted in FIG. 12B.

FIG. 12E is a graph showing the relative quantification of the different isoforms produced by the RAB7L1 minigene. The numbers correspond to the different isoforms expected from the minigene and shown in FIG. 12A.

FIG. 12F is a bar graph showing that alternatively spliced RAB7L1 (AT) does not functionally rescue LRRK2 G2019S neurite length assay. Total neurite length of primary rat cortical neurons transfected with expression vectors as indicated. (n=20 per group; ***p<0.001 vs vector alone by ANOVA followed by Bonferroni correction).

FIG. 13 is a photographic image of an immunoprecipitation using an anti-LRRK2 antibody from whole brain lysates of nontransgenic (NT), LRRK2 wild type transgenic (WT). LRRK2 R1441C (RC) transgenic, or LRRK2 knockout (−/−) mice. IB was subsequently performed for VPS35 and betaactin. VPS35 but not beta-actin were co-precipitated with LRRK2. Neither VPS35 nor beta-actin were immunoprecipitated by a control IgG antibody, or from LRRK2 KO mice.

### DETAILED DESCRIPTION

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Parkinson's disease (PD) is a common neurodegenerative disorder of aging, characterized by slowed movements and a distinctive tremor at rest (Lang and Lozano, 1998). Defining pathological features of the disease include neurodegeneration that is most prominent among midbrain dopamine neurons (DNs) in the Substantia Nigra (SN) and Lewy body protein aggregates that are composed in part of alpha-Synuclein (aSyn) protein. As the course of PD is thought to last decades, and as at the time of autopsy the vast majority of DNs are long lost, the molecular pursuit of initial etiological events has proven difficult.

In rare inherited familial forms of PD, specific causative mutations have been identified, and this has significantly advanced the field (Abeliovich and Beal, 2006; Hardy et al., 2006). For instance, autosomal dominantly inherited mutations in aSyn, including missense mutations and triplication of the locus, lead to a familial PD variant, implicating aSyn directly in the disease process. Another familial genetic cause of PD is the presence of autosomal dominantly inherited mutations in the Leucine rich-repeat kinase-2 (LRRK2) protein, which encodes a large multidomain protein with GTPase and kinase activities. Although the precise functions of aSyn and LRRK2 in neurons remain to be determined, both proteins have been broadly implicated in intraneuronal protein sorting. aSyn mutations have been reported to modify synaptic vesicle kinetics (Abeliovich et al., 2000) as well as trafficking to the Golgi apparatus in a variety of model systems (Cooper et al., 2006; Thayanidhi et al., 2010), whereas LRRK2 mutations are implicated in defective lysosomal protein degradation and macroautophagy, which is a cellular process that delivers cytosolic proteins and protein agregates to the lysosome (Dodson et al., 2012; Heo et al., 2010; MacLeod et al., 2006), and Golgi Apparatus integrity (Stafa et al., 2012). The recent identification of rare autosomal dominant familial PD mutations in VPS35 (Vilarino-Guell et al., 2011; Zimprich et al., 2011), which encodes a component of the retromer complex that guides protein sorting from the endosome-lysosome degradation pathway retrogradely to the Golgi Apparatus (Bonifacino and Hurley, 2008; Seaman, 2009; Seaman et al., 1998), suggests that defective protein sorting in vesicular compartments may play a role in PD.

Several genome-wide association studies (GWAS) have described common genetic variants (at single nucleotide polymorphisms; SNPs) that modify PD risk in non-familial ‘sporadic’ cases (Hamza et al., 2010; Simon-Sanchez et al., 2009). Strikingly, a subset of these common variants lie within genomic loci previously associated with familial disease, such as aSyn or LRRK2, supporting the notion that common pathogenic pathways underlie familial and sporadic forms of PD. However, mechanisms that underlie the impact of non-familial genetic loci on PD risk, or that relate the functions of such loci to familial PD genes, remain unclear.

Described herein is a series of human brain transcriptome, human genetic, and cell biological studies, that together implicate a PD-associated genetic and cellular pathway. RAB7L1—one of 5 genes within the PARK16 non-familial PD risk-associated locus—functions together with LRRK2 to impact non-familial PD risk in the human population. This genetic interaction is apparent even in unaffected individuals who carry both risk alleles, as quantified in terms of a broad transcriptomic analysis of brain gene expression. Similarly, these genes together modify neuronal survival and neurite integrity in model systems. At a cellular level, defects in this PD-associated RAB7L1-LRRK2 pathway lead to abnormal lysosomal structures and defective retromer complex function, that normally links the endolysosomal protein degradation system with the Golgi apparatus (Bonifacino and Hurley, 2008; Seaman. 2009; Seaman et al., 1998). Consistent with a role for such cellular defects in disease pathology, mutations in a retromer complex component, VPS35, have recently been associated with rare forms of autosomal dominantly inherited familial PD (Vilarino-Guell et al., 2011; Zimprich et al., 2011).

Molecules of the Invention

As used herein, a “RAB7L1 molecule” refers to a RAB7L1 protein, or a fragment thereof. A “RAB7L1 molecule” can also refer to a nucleic acid (including, for example, genomic DNA, complementary DNA (cDNA), synthetic DNA, as well as any form of corresponding RNA) which encodes a polypeptide corresponding to a RAB7L1 protein, or fragment thereof. For example, a RAB7L1 molecule can comprise the nucleic acid sequences shown in SEQ ID NOS: 1, 2, 3, 4, or 5, or comprise the amino acid sequences shown in SEQ ID NOS: 6, 7, 8, or 26. For example, a RAB7L1 molecule can be encoded by a recombinant nucleic acid encoding a RAB7L1 protein, or fragment thereof. The RAB7L1 molecules of the invention can be obtained from various sources and can be produced according to various techniques known in the art. For example, a nucleic acid that encodes a RAB7L1 molecule can be obtained by screening DNA libraries, or by amplification from a natural source. A RAB7L1 molecule can include a fragment or portion of a RAB7L1 protein. A RAB7L1 molecule can include a variant of the above described examples, such as a fragment thereof. Such a variant can comprise a naturally-occurring variant due to allelic variations between individuals (e.g., polymorphisms), mutated alleles, or alternative splicing forms (e.g. SEQ ID NOS: 2-5). In one embodiment, a RAB7L1 molecule is encoded by a nucleic acid variant of the nucleic acid having the sequence shown in SEQ ID NOS: 1, 2, 3, 4, or 5 wherein the variant has a nucleotide sequence identity to SEQ ID NOS: 1, 2, 3, 4, or 5 of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In another embodiment, a variant of the RAB7L1 protein comprises a protein or polypeptide encoded by a RAB7L1 nucleic acid sequence, such as the sequence shown in SEQ ID NOS: 1, 2, 3, 4, or 5.

As used herein, a “LRRK2 molecule” refers to a LRRK2 protein, or a fragment thereof. A “LRRK2 molecule” can also refer to a nucleic acid (including, for example, genomic DNA, complementary DNA (cDNA), synthetic DNA, as well as any form of corresponding RNA) which encodes a polypeptide corresponding to a LRRK2 protein, or fragment thereof. For example, a LRRK2 molecule can comprise the nucleic acid sequences shown in SEQ ID NOS: 9, or 10, or comprising the amino acid sequences shown in SEQ ID NO: 11, 27, or 28. For example, a LRRK2 molecule can be encoded by a recombinant nucleic acid encoding a LRRK2 protein, or fragment thereof. The LRRK2 molecules of the invention can be obtained from various sources and can be produced according to various techniques known in the art. For example, a nucleic acid that encodes a LRRK2 molecule can be obtained by screening DNA libraries, or by amplification from a natural source. A LRRK2 molecule can include a fragment or portion of a LRRK2 protein. A LRRK2 molecule can include a variant of the above described examples, such as a fragment thereof. Such a variant can comprise a naturally-occurring variant due to allelic variations between individuals (e.g., polymorphisms), mutated alleles, or alternative splicing forms. In one embodiment, a LRRK2 molecule is encoded by a nucleic acid variant of the nucleic acid having the sequence shown in SEQ ID NOS: 9, or 10 wherein the variant has a nucleotide sequence identity to SEQ ID NOS: 9 or 10 of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In another embodiment, a variant of the LRRK2 protein comprises a protein or polypeptide encoded by a LRRK2 nucleic acid sequence, such as the sequence shown in SEQ ID NOS: 9 or 10.

As used herein, a “VPS35 molecule” refers to a VPS35 protein, or a fragment thereof. A “VPS35 molecule” can also refer to a nucleic acid (including, for example, genomic DNA, complementary DNA (cDNA), synthetic DNA, as well as any form of corresponding RNA) which encodes a polypeptide corresponding to a VPS35 protein, or fragment thereof. For example, a VPS35 molecule can comprise the nucleic acid sequences shown in SEQ ID NOS: 12 or 13, or comprising the amino acid sequences shown in SEQ ID NO: 14. For example, a VPS35 molecule can be encoded by a recombinant nucleic acid encoding a VPS35 protein, or fragment thereof. The VPS35 molecules of the invention can be obtained from various sources and can be produced according to various techniques known in the art. For example, a nucleic acid that encodes a VPS35 molecule can be obtained by screening DNA libraries, or by amplification from a natural source. A VPS35 molecule can include a fragment or portion of a VPS35 protein. A VPS35 molecule can include a variant of the above described examples, such as a fragment thereof. Such a variant can comprise a naturally-occurring variant due to allelic variations between individuals (e.g., polymorphisms), mutated alleles, or alternative splicing forms. In one embodiment, a VPS35 molecule is encoded by a nucleic acid variant of the nucleic acid having the sequence shown in SEQ ID NOS: 12, or 13 wherein the variant has a nucleotide sequence identity to SEQ ID NOS: 12, or 13 of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In another embodiment, a variant of the VPS35 protein comprises a protein or polypeptide encoded by a VPS35 nucleic acid sequence, such as the sequence shown in SEQ ID NOS: 12 or 13.

The nucleic acid can be any type of nucleic acid, including genomic DNA, complementary DNA (cDNA), synthetic or semi-synthetic DNA, as well as any form of corresponding RNA. For example, a nucleic acid encoding a RAB7L1, a LRRK2, or a VPS35 protein can comprise a recombinant nucleic acid encoding such a protein. The nucleic acid can be a non-naturally occurring nucleic acid created artificially (such as by assembling, cutting, ligating or amplifying sequences). It can be double-stranded or single-stranded.

The invention further provides for nucleic acids that are complementary to a RAB7L1, a LRRK2, or a VPS35 molecule. Complementary nucleic acids can hybridize to the nucleic acid sequence described above under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

According to the invention, protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture. In one embodiment, a RAB7L1, a LRRK2, or a VPS35molecule can be modified with an amino acid sequence inserted as a carboxyl terminal fusion. For example, carboxyl terminal fusions may be used to increase the stability of a RAB7L1, a LRRK2, or a VPS35 molecule.

In one embodiment, a RAB7L1 molecule comprises a protein or polypeptide encoded by a nucleic acid sequence encoding a RAB7L1 protein, such as the sequences shown in SEQ ID NOS: 6, 7, 8, or 26. In another embodiment, the polypeptide can be modified, such as by glycosylations and/or acetylations and/or chemical reaction or coupling, and can contain one or several non-natural or synthetic amino acids. An example of a RAB7L1 molecule is the polypeptide having the amino acid sequence shown in SEQ ID NOS: 6, 7, 8, or 26. Such variants can include those having at least from about 46% to about 50% identity to SEQ ID NOS: 6, 7, 8, or 26 or having at least from about 50.1% to about 55% identity to SEQ ID NOS: 6, 7, 8, or 26, or having at least from about 55.1% to about 60% identity to SEQ ID NOS: 6, 7, 8, or 26, or having from at least about 60.1% to about 65% identity to SEQ ID NOS: 6, 7, 8, or 26, or having from about 65.1% to about 70% identity to SEQ ID NOS: 6, 7, 8, or 26, or having at least from about 70.1% to about 75% identity to SEQ ID NOS: 6, 7, 8, or 26, or having at least from about 75.1% to about 80% identity to SEQ ID NOS: 6, 7, 8, or 26, or having at least from about 80.1% to about 85% identity to SEQ ID NOS: 6, 7, 8, or 26, or having at least from about 85.1% to about 90% identity to SEQ ID NOS: 6, 7, 8, or 26, or having at least from about 90.1% to about 95% identity to SEQ ID NOS: 6, 7, 8, or 26, or having at least from about 95.1% to about 97% identity to SEQ ID NOS: 6, 7, 8, or 26, or having at least from about 97.1% to about 99% identity to SEQ ID NOS: 6, 7, 8, or 26. In another embodiment, a RAB7L1 molecule can be a fragment of a RAB7L1 protein.

In one embodiment, a LRRK2 molecule comprises a protein or polypeptide encoded by a nucleic acid sequence encoding a LRRK2 protein, such as the sequences shown in SEQ ID NOS: 11, 27, or 28. In another embodiment, the polypeptide can be modified, such as by glycosylations and/or acetylations and/or chemical reaction or coupling, and can contain one or several non-natural or synthetic amino acids. An example of a LRRK2 molecule is the polypeptide having the amino acid sequence shown in SEQ ID NOS: 11, 27, or 28. Such variants can include those having at least from about 46% to about 50% identity to SEQ ID NOS: 11, 27, or 28 or having at least from about 50.1% to about 55% identity to SEQ ID NOS: 11, 27, or 28, or having at least from about 55.1% to about 60% identity to SEQ ID NOS: 11, 27, or 28, or having from at least about 60.1% to about 65% identity to SEQ ID NOS: 11, 27, or 28, or having from about 65.1% to about 70% identity to SEQ ID NOS: 11, 27, or 28, or having at least from about 70.1% to about 75% identity to SEQ ID NOS: 11, 27, or 28, or having at least from about 75.1% to about 80% identity to SEQ ID NOS: 11, 27, or 28, or having at least from about 80.1% to about 85% identity to SEQ ID NOS: 11, 27, or 28, or having at least from about 85.1% to about 90% identity to SEQ ID NOS: 11, 27, or 28, or having at least from about 90.1% to about 95% identity to SEQ ID NOS: 11, 27, or 28, or having at least from about 95.1% to about 97% identity to SEQ ID NOS: 11, 27, or 28, or having at least from about 97.1% to about 99% identity to SEQ ID NOS: 11, 27, or 28. In another embodiment, a LRRK2 molecule can be a fragment of a LRRK2 protein.

In one embodiment, a VPS35 molecule comprises a protein or polypeptide encoded by a nucleic acid sequence encoding a VPS35 protein, such as the sequences shown in SEQ ID NO: 14. In another embodiment, the polypeptide can be modified, such as by glycosylations and/or acetylations and/or chemical reaction or coupling, and can contain one or several non-natural or synthetic amino acids. An example of a VPS35 molecule is the polypeptide having the amino acid sequence shown in SEQ ID NO: 14. Such variants can include those having at least from about 46% to about 50% identity to SEQ ID NO: 14 or having at least from about 50.1% to about 55% identity to SEQ ID NO: 14, or having at least from about 55.1% to about 60% identity to SEQ ID NO: 14, or having from at least about 60.1% to about 65% identity to SEQ ID NO: 14, or having from about 65.1% to about 70% identity to SEQ ID NO: 14, or having at least from about 70.1% to about 75% identity to SEQ ID NO: 14, or having at least from about 75.1% to about 80% identity to SEQ ID NO: 14, or having at least from about 80.1% to about 85% identity to SEQ ID NO: 14, or having at least from about 85.10% to about 90% identity to SEQ ID NO: 14, or having at least from about 90.1% to about 95% identity to SEQ ID NO: 14, or having at least from about 95.1% to about 97% identity to SEQ ID NO: 14, or having at least from about 97.1% to about 99% identity to SEQ ID NO: 14. In another embodiment, a VPS35 molecule can be a fragment of a VPS35 protein.

In one embodiment, a RAB7L1, a LRRK2, or a VPS35 molecule, according to the methods described herein can be administered to a subject as a recombinant protein. In another embodiment, a RAB7L1, a LRRK2, or a VPS35 molecule, can be administered to a subject as a modified recombinant protein. In a further embodiment, a RAB7L1, a LRRK2, or a VPS35 molecule, according to the methods described herein can be administered to a subject by delivery of a nucleic acid encoding a RAB7L1, a LRRK2, or a VPS35 protein, or fragment thereof. For example, nucleic acids can be delivered to a subject using a viral vector.

Polypeptides can be susceptible to denaturation or enzymatic degradation in the blood, liver or kidney. Accordingly, polypeptides can be unstable and have short biological half-lives. Polypeptides can be modified to increase their stability, for example, a fusion protein can be generated for increased stability and to cause a longer biological half-life to the polypeptides in circulation.

The term “biological half-life” is the time required for the activity of a substance taken into the body to lose one half its initial pharmacologic, physiologic, or biologic activity.

The invention provides for a nucleic acid encoding a RAB7L1 protein, or fragment thereof.

For example, the human genomic nucleotide sequence corresponding to the sense strand of the human RAB7L1 gene is depicted in SEQ ID NO: 1 (9752 bp). Sequence information related to RAB7L1 is accessible in public databases by GenBank Accession number NC_000001.10, 205737114, 205744615, complement (nucleotide).

SEQ ID NO: 1:

| | |
|---|---|
| 1 | ctgaaagaaa aagataatca tcagagaagt acggggatga caaagaaaga acagcgtcat |
| 61 | agaaggcata agggaaacaa atgtcaagga gtggtcaact atgtcaaaac gaataagaac |
| 121 | agagaaaact ggatccttaa agatgagtag cttgaactaa cctcctgtac ctgggtaacg |
| 181 | aacattctgg gcaaaatata ttgtgaacca tgctgatgtt ctttcgccac aaaacataag |
| 241 | tgatagcctc tctatcaaga gacctagatt tcctagtgtc tggtcctggg ctgttttccc |
| 301 | atggccccgc tggttctctt cctctattcc tagaaaagta tagaaaatgg agctctcttt |
| 361 | gcttgccatt gagctcatgt cctgccaact tctacccaag tgacaggtag gtcagtaaag |
| 421 | ttacaggctg agaaattaaa attatttgcc aaacactagt tttatgatta tctattaaca |
| 481 | acagccacaa caaatcaaga aggagtccca gtgtatttca attagcttct agctcaagtg |
| 541 | atacatgagt agtatggcta gaataggcat tgtagaagta tgaatggaga actgtctctt |
| 601 | gttttctgtc catctttatt gttattgctg gatattaaat gctgtaattt ttggcatagg |
| 661 | tttttttcca aagactggtc tcttgggtca agttttcctg aacttgcctg gtattgggca |
| 721 | agttcacaca gaaaatatga gtcatttcac aaagtcctgg gcttgtctat ctacttgaag |
| 781 | gaataggaaa tggacttcac ttggagcatg atggatgagg cttgggcttt tactaatgaa |
| 841 | ataagtttca atcttaactc agttaaacct ttaaagacag gagaaaacta tctggggatg |
| 901 | gattcaggcc agtgagatca cgttgcacaa atctcccctt aagggatctt tactgaccta |
| 961 | gaattgtttc aaaataagat tccaaactgt cgagggagtt tgaagccttg aaacttggtg |
| 1021 | taggtttgct taaaacaaaa taacgaatat actacatttc ccaggaggct tcgcggtgag |
| 1081 | cctccgcact cggctggttc tctttaccgc gaggaaagct gggaaatgta gtgccacagg |
| 1141 | caaccctgca cgtgacgctt gcggaggaag gggagagaga ggcgcgcggg agggcgtcta |
| 1201 | gggaatcgag gtgccggctg ctccttcctc acaatttggt ttgtgctgca aggggagggt |
| 1261 | ccccatcatc tggccccagt ggtgtaagga gctgactggg attcagtcac tgacttggag |
| 1321 | ccgctcgggg gaagtcccgg tgggtgaggt tccgcggcgc ctggtccagt ttctcggcag |
| 1381 | tcaggccagg agggggtggg gaaggtgcga acagccgggg gctcggagct cgcgccctgc |
| 1441 | gccccaccca ggttgcggcc gcccggggag gaagcgcgta taggttgagt gcaaagtttc |
| 1501 | cttttttgct ttctcgtcag agcagcccag acaggcggtg ggtgggaatg cctcacttca |
| 1561 | gtttgaagag ggtccggatc caaaggggtt aaaacgagcg acccccgatc cccgaccaca |
| 1621 | cttcccgcct ccctaaaacg cacaccccgc tagccatggg cagccgcgac cacctgttca |
| 1681 | aagtgctggt ggtgggggac gccgcagtgg gcaagacgtc gctggtgcag cgatattccc |
| 1741 | aggacagctt cagcaaacac tacaagtcca cggtgggagg tgagacgtct cgggggtggg |
| 1801 | gagggggagg gggaggagga ggccccgacc gcggactcga gctggggacc caagtggggt |
| 1861 | ggggatttgc tgtcggttgt tgggtccttc ttcgggtcag gaagtactgt ctgggaacct |
| 1921 | agatggtgta attgccgggt gtttggttta cacgcctctc ttgagtttcc cgttagattt |
| 1981 | tcccaaactg agcccactgc caggagaaag gtaagagcaa aggcgtgaag atgagaaata |
| 2041 | gggaagggag gatgaggatg agcaattgct gtttattgca ggcgtactgt gaacttaagt |
| 2101 | ctccagcctt tcagctgtcc ggcaaatctc tccaatcagc agaagcagct gagatcctct |
| 2161 | gtggggcctc ctggttaggt gccggctggg gctgtagtcc tgcccctctg ttgagggagg |
| 2221 | aactgcaagg tgtttaacct ggcttcagtg ggagaacgca ggaagcggaa ctggtctgaa |
| 2281 | ggactgtagt tttgttgtgg ggctgtgggt gggatggaag acacaaaatc cccgaacgag |
| 2341 | ccccttggct acgcagtcag atgcccatga gggctgatga gctgtgtttt tgtccgttga |
| 2401 | ttgcttaaat aaaggtaacc aaggaattct tccaagaatg tgaaatatac cacttctctg |
| 2461 | acctatcttc ccaccacttt tttcctcaaa ctggagaagg tagggaggaa ggggacaccg |
| 2521 | agttcattca tttctttccc gggtgtggcc tttggactca actatttttt cagaggcaga |
| 2581 | aatgccttat tgttaattag aaggtattta gctaggaaga gctggtgatg ggggagagta |
| 2641 | acttaaaatt ttatttctct ctgttaaaag aacttgtttc aaggggggaa aactcgtacc |
| 2701 | aatggaaaaa ttgaaaatgt gtgctcctgt tcatatggaa gtttaaaaag aaattaacaa |
| 2761 | acatttacat tgcaattaaa acattctcct taagaacccc cagtagggcc cagtaccgtg |
| 2821 | gttcatactt gcaatcccag cactttagga ggctgaggcg ggaggattgc ttgagcccag |
| 2881 | gagttcgaga ccagccatgg gaaacatggc aagaccccca cctctacaaa aattaaagaa |
| 2941 | gaaagtagcc gtggtcccag ctactcggga aactgagacg agaggattgc ttgaactggg |
| 3001 | gaggtcgagg cttcagtgag ccccgttcgc accactcact gcactgtagc ctgggcaaca |
| 3061 | gagtgagacc ctgtctcaaa aacaaaggaa caaaaaattt aagtctggca aaattaaagg |
| 3121 | ttcaaagttt cagacactct atcctgaccg ctctgccctc ccactcctaa ataatataag |
| 3181 | ggtccctatt tcacggattt gggttaaaag tggcctgcta ttaatagtta atttttaaca |
| 3241 | attgctgtag ttttccttta gcaaagaata taattggcta attttttttt cagtgtttag |
| 3301 | tctaaatttc ccttagcttt gattcctgtc acagatgtga aagataatat ttccaaacct |
| 3361 | cagaaattac tctgtttctc tccttagcca aaaggctttc cccatggaat acttactcag |
| 3421 | ccttaaggag tctcagcacc tgcagacttg gtggtagatt ccagcaggac tcttcagcaa |
| 3481 | catccacaca gtgtcttctt cttgggactc ctctgggacc ttttccattt attccattct |
| 3541 | tgcccaagat aatacctgca accttatggt ggatgtagag ttccccaggg tgtgttcttt |
| 3601 | tcataataca atttgattag aaaccaacag tgaaattgaa tgtggaagat acgaaagcaa |
| 3661 | ccacacttgt ctcatttccc ccttctcccc catcacccct tccctgagaa aaccccctaa |
| 3721 | atctcccaga aactatttaa cctgagaatg agaattgttt tcatagttac tcccaccccc |
| 3781 | agtcttggag acaaacagta gcaaataaat gagcttgaca gtagagtaaa agctggaaac |
| 3841 | ccctttggag agtgagtatg aatcatccat ctcacactaa atacacgcat gtggactcat |
| 3901 | ctcagtgaat tgagggctta aagttaaaca tatgggattg gagttgtgtg tccatagggt |
| 3961 | ttcactgcct atttgatttg agtttatccc tattaatttt ttacagtgaa attttattaa |
| 4021 | agtataatgt acatatattt tcagtggatt ttgctctgaa ggttctccag tggtctgact |
| 4081 | acgagatagt gcggcttcag ctgtgggata ttgcaggtaa agtgggaggg ctagatagga |
| 4141 | acaagggaag aaattttgat ttaactagaa agctctgagg aaaaaatggg gcagcaaagc |
| 4201 | cagaaattgt ttttctgaag ctggaatgat tgagctgctc tgaggacaca tgagctgaat |
| 4261 | aggtgcagag cttttgtaga tgttttgccc tgaatagatc tggagcagtc tctatgtgga |
| 4321 | tggaggggct ggcaaggtgg tggattgtcc agactatagg tggacactcc cacagcaggg |
| 4381 | gagggtgctt tttctaggtt taaggctgac cttggtgaaa aacaagatcc ccagaaaata |
| 4441 | gatacagtaa aaatagtgaa tccttactat gtgccaagca ttcttctaag tattttaaat |
| 4501 | tcactgactc attaatcctc acatcaaccc tatcataaaa gctactgctt ttggagaatt |
| 4561 | gcttgaacct gggaggtgga ggttgcagtg agcctagatg gcaccactgc actccagcct |
| 4621 | gggcaacagg agtgcaggaa actgtttcaa aaaaaaaaaa aaaaagcgac tgcttttatc |
| 4681 | ttcactttac aaatgagaaa tctgaagcac tgagagcttg atttgcccga agtcaaatag |
| 4741 | tatgcagcag agcagggatt tggactggag caatctgggt gcggaatctg ccttggaaat |
| 4801 | aattgggctt cattatccct ttatgggata gaaaaccaag agtcagaggg ggaaagtgag |
| 4861 | tggccccatc tgacacatat gcttactgcc ctcttctaca aggaatgggg agaggccggc |
| 4921 | tcttgggtca ccttgagaac tctccccttt ctccccaggg caggagcgct tcacctctat |
| 4981 | gacacgattg tattatcggg atgcctctgc ctgtgttatt atgtttgacg ttaccaatgc |
| 5041 | cactaccttc agcaacagcc agaggtggaa acaggaccta gacagcaagc tcacactacc |
| 5101 | caatggagag ccggtgccct gcctgctctt ggccaacaag gtatgtggcc aatctcagaa |
| 5161 | aatggtccct agcctctatc tgtggttaga aaggaagtaa aatgctctct gattctgggc |
| 5221 | atccatttcc ccttcttaag ttggcaaaat catctctacc tccttcaaag gatgttgagg |
| 5281 | gtgagtgaga ctttctttct tttttttttt tttgagacgg agtctcgctc tgttgctcag |
| 5341 | gctggagtgc agtggcgcga tctcggctca ctgccagctc tgcctcccag gttcacgcca |
| 5401 | ttctcttgcc tcagcctccc gagtagctgg gactacaggt gcccaccacc acacccagct |
| 5461 | aatttttttg tatttttagt agagactggg tttcattgtg ttagccagga tggtctcgct |
| 5521 | ctcctgacct catgatctgc ccgcctcagc ctcccaaagt gctgggatta caggcgtgag |
| 5581 | ccactgcgcc cagccaaggg tgagactttc tttgggaaga atgttgatta tagatttccg |
| 5641 | atgctggtgt tcttatccat tggtggaaat atgtttaaaa tccacagtat ctgagccata |
| 5701 | ttctttctta gttgcaggaa gtttatgata ctgcctttta ttttttctct ttgctagtgt |
| 5761 | gatctgtccc cttgggcagt gagccgggac cagattgacc ggttcagtaa agagaacggt |
| 5821 | ttcacaggtt ggacagaaac atcagtcaag gagaacaaaa atattaatga ggctatgagg |
| 5881 | taagtagctc atgctgatag gcatgcagat gtatccatct tccactgtgg ccctgtggac |
| 5941 | cctcttttct tatttctgag ccaacaatga cagactgagc cattggaatg ctagccccta |
| 6001 | aaggtcaggg atactgtctt tcctgagata actggggaca aaggggcatt taaaccttct |
| 6061 | ggctttacca aatactctct gatgataagt cagactacat ctgccatttc tgcttttcct |
| 6121 | ccaaaataag cttctcaggc ttattttttg tcttttagag tcctcattga aaagatgatg |
| 6181 | agaaattcca cagaagatat catgtctttg tccacccaag gggactacat caatctacaa |
| 6241 | accaagtcct ccagctggtc ctgctgctag tagtgtttgg cttattttcc atcccagttc |
| 6301 | tgggaggtct tttaagtctc ttccctttgg ttgcccacct gacaatttta ttaagtacat |
| 6361 | ttgaattgtc tcctgactac tgtccagtaa ggaggcccat tgtcacttag aaaagacacc |
| 6421 | tggaacccat gtgcatttct gcatctcctg gattagcctt tcacatgttg ctggctcaca |
| 6481 | ttagtgccag ttagtgcctt cggtgtaaga tcttctcatc agccctcaat ttgtgatccg |
| 6541 | gaattttatg agaaggatta gaaatcagca cctgcgtttt agagatcata attctcacct |
| 6601 | acttctgagc ttatttttcc atttgatatt cattgatatc atgacttcca attgagagga |
| 6661 | aaatgagatc aaatgtcatt tcccaaattt cttgtaggcc gttgtttcag attctttctg |
| 6721 | tcttggaatg taaacatctg attctggaat gcagaaggag ggggtctggg catctgtgga |
| 6781 | tttttggcta ctagaagtgt cccagaagtc actgtatttt tgaaacttct aacgtcataa |
| 6841 | ttaagtttct cttgtcttgg catcaagaat agtcaagttt tttggccggg catggtggct |
| 6901 | catgcctgta atcccagcac ttggggaggc caaggcaggc ggatcacatg aggccaggaa |
| 6961 | ttcgagacca acctggtcag catggcaaaa ccccgtctct actaaaagta caaaaattag |
| 7021 | ccaggcgtga tggcacgtgt ctgtaatccc agctactctg gagactgagg tgggagaatc |
| 7081 | gcttgagact gggaggcaga ggttgcagtg aaccgagatc atgccaccgc acttcagcct |
| 7141 | gggtgacaga gaaggactcc gtctcaaaaa aaaaagaaaa aagaatagtc atttttaaac |
| 7201 | tacctatctc atgcaatgaa agcattttct tccacaaaga gcttaatcct catgatagga |
| 7261 | ttgcctagtg tctcccattt gcaggtttct gggttgatgt cttaatgcat aatactgcaa |
| 7321 | gtgacatcag ctggctgtga tgcttcgaaa taggtctgct cctcacagct ttgggaatct |
| 7381 | gaatggaaga agaaaagaga gaagttaaca acctccactg gggcaacttt gtgaacacgt |
| 7441 | aggcacttag tcataggaaa catattatgt gcaggtccta gcctggggga ggaaagtaga |
| 7501 | tagacagaaa atcattaggt aatttaagta ctaaattggg cagggctttt tagtatcaaa |
| 7561 | tcactactag accatttaat ttgttaaatt atctctagga tggtgattta taacctaccc |
| 7621 | aaagttatcg atattcttag taaactctga ggcctgaagt tctgtgatag accttaaata |
| 7681 | agtgtcctaa gtcagtggtt cccaaatctg gctggtcggg aatacctggg aagtttgtta |
| 7741 | aaatttttta aaaatgtttt aagatttttg ggtcctgagc cagccgtggt ggctcacacc |
| 7801 | tgtaatccca gcactttggg aggctgaggc aggtggatcg cctgaggtca ggagttcaag |
| 7861 | atcaacctgg ccaacatact gaaaccccgt ctctactaaa aataagaaaa attagctggg |
| 7921 | cgtggtggcg ggcacctgta atcccagcta cttgggagac tgaggcagga gaatcgcttg |
| 7981 | aacctgggag ttagaggttg cagtgagctg agatcacacc attgcgcttc agcctgggca |
| 8041 | acaagagtga aactccatct ccaaaaaaaa aaaacaaaaa gaaaaagatt tttgggtccc |
| 8101 | gacctcaaac ctactgaatc agaatttcta gggatgaagc ctaggaatgt gttgttgttt |
| 8161 | tcagagcttc cctggtgatt gtgataagcc tggtttggaa accattgctg gagaactttg |
| 8221 | taaagataca gagacccaga ccttttgtat ttacatttaa atacaaatac aaatcctggg |
| 8281 | tttctatata ttctgttagc ttttcaggtg attctgctac acagacgttg aaaaccactg |
| 8341 | ccctaagaaa gagatcagag gccacatatc agagagaaaa gggaccaaac cttcggtggt |
| 8401 | ttgttgtgtg tcgttttaat gccaattatt ttaacttgca cagtcttctg aaaccttgta |
| 8461 | ttaatagttc tcttttgtat taccattttc aggtagggtt ttgatcacta tgattctgaa |
| 8521 | gataatagtg aaatagtgaa tttcattgat atgaagagat aattgatttt cattcattgg |
| 8581 | tttgaacacc tgcaaaatca caaataaatg agaactaagt cttgtattca tggtggttat |
| 8641 | tggcctttaa tgtgagtttg tcaaagtgct gttttatact gatagctcaa gaggattgcg |
| 8701 | gaaatggaga ctttattttt taaatacctt tttctaaatt ttcaattcaa ggggtacgtg |
| 8761 | tgcaagtttg ttacatggat atattgtgtg atgctaaagc ttgggtttct gcttagcctg |
| 8821 | tctcccaagt agtgaacata gcacccagtc ggcacttttt caacgcttac tccctctctc |
| 8881 | cccacttttg ggagtctcca gtgtctgttg ttcccatctt tatgtgtgtg cccaatgttt |
| 8941 | acctcccact tacaagtgag aaatgtggtt aaataccttc ttttaaaatg aaggtaaata |
| 9001 | ttattctgtt tacctgttaa cttactaata ccaattgctt agatccatct gcctagtacc |
| 9061 | aattcagtac ttgatggaac atgccaaata ctacttggaa acgtgacctt tttgaaaaac |
| 9121 | tgaatgtgct tgcttttccc atttgctgac tcaggattta cctgcctttt caagcattgt |
| 9181 | tagaggctgg ccagccagcc agcccttctg gatattctct cccaactggt gaacattata |
| 9241 | ggcctgaaac taattaaaga aggaactgaa atatacctaa ttcctcttcg tgcccctatc |
| 9301 | cataagagaa aagcaattat gattaggaaa aataaaatat ttacatgaat ggaaatgcga |
| 9361 | tgctactaaa aataattagg tcttatcact cggatgctac ctctttggtg cattcaaaag |
| 9421 | aggtagcgtc caaaattgtt tggaacgtag atatcgtagc ccctccctag aggaggtcct |
| 9481 | acatttgaga caagatgcct ctccaaccac tcaggtggct tttggtcaaa aaagaaaaca |
| 9541 | gttcccccag agtgttctca gaaaccctaa gggagggctg agaacacagc tgtggggtag |
| 9601 | tgtctgccct gactttcttc tttgaacaga agaaatttgg tcacttagct gagtgaccaa |
| 9661 | ataagtggag cagaggagat tgtaggtcag ggaaggccct ttagcagtaa agaatggtct |
| 9721 | gctgctttat cactactatt gcgccagaat ta |

For example, the nucleotide sequence corresponding to the mRNA of the human RAB7L1 (transcript variant 1) is depicted in SEQ ID NO:2 (3324 bp), wherein the underscored bolded “ATG” denotes the beginning of the open reading frame. Nucleotides 1130-1238, 1526-1779, 4045-4116, 4959-5140, 5758-5879, and 6159-8626 of SEQ ID NO: 1 can be spliced together to form RAB7L1 (transcript variant 1). Sequence information related to RAB7L1 (transcript variant 1) is accessible in public databases by GenBank Accession number NM 003929.2 (nucleotide).

SEQ ID NO: 2:

| | |
|---|---|
| 1 | agtgccacag gcaaccctgc acgtgacgct tgcggaggaa ggggagagag aggcgcgcgg |
| 61 | gagggcgtct agggaatcga ggtgccggct gctccttcct cacaatttgg tttgtgctgc |
| 121 | aaggggaggg tccccatcat ctggccccag tggtgtaagg agctgactgg gattcagtca |
| 181 | ctgacttgga gccgctcggg ggaagtcccg cccagacagg cggtgggtgg gaatgcctca |
| 241 | cttcagtttg aagagggtcc ggatccaaag gggttaaaac gagcgacccc cgatccccga |
| 301 | ccacacttcc cgcctcccta aaacgcacac cccgctagcc atgggcagcc gcgaccacct |
| 361 | gttcaaagtg ctggtggtgg gggacgccgc agtgggcaag acgtcgctgg tgcagcgata |
| 421 | ttcccaggac agcttcagca aacactacaa gtccacggtg ggagtggatt ttgctctgaa |
| 481 | ggttctccag tggtctgact acgagatagt gcggcttcag ctgtgggata ttgcagggca |
| 541 | ggagcgcttc acctctatga cacgattgta ttatcgggat gcctctgcct gtgttattat |
| 601 | gtttgacgtt accaatgcca ctaccttcag caacagccag aggtggaaac aggacctaga |
| 661 | cagcaagctc acactaccca atggagagcc ggtgccctgc ctgctcttgg ccaacaagtg |
| 721 | tgatctgtcc ccttgggcag tgagccggga ccagattgac cggttcagta aagagaacgg |
| 781 | tttcacaggt tggacagaaa catcagtcaa ggagaacaaa aatattaatg aggctatgag |
| 841 | agtcctcatt gaaaagatga tgagaaattc cacagaagat atcatgtctt tgtccaccca |
| 901 | aggggactac atcaatctac aaaccaagtc ctccagctgg tcctgctgct agtagtgttt |
| 961 | ggcttatttt ccatcccagt tctgggaggt cttttaagtc tcttcccttt ggttgcccac |
| 1021 | ctgacaattt tattaagtac atttgaattg tctcctgact actgtccagt aaggaggccc |
| 1081 | attgtcactt agaaaagaca cctggaaccc atgtgcattt ctgcatctcc tggattagcc |
| 1141 | tttcacatgt tgctggctca cattagtgcc agttagtgcc ttcggtgtaa gatcttctca |
| 1201 | tcagccctca atttgtgatc cggaatttta tgagaaggat tagaaatcag cacctgcgtt |
| 1261 | ttagagatca taattctcac ctacttctga gcttattttt ccatttgata ttcattgata |
| 1321 | tcatgacttc caattgagag gaaaatgaga tcaaatgtca tttcccaaat ttcttgtagg |
| 1381 | ccgttgtttc agattctttc tgtcttggaa tgtaaacatc tgattctgga atgcagaagg |
| 1441 | agggggtctg ggcatctgtg gatttttggc tactagaagt gtcccagaag tcactgtatt |
| 1501 | tttgaaactt ctaacgtcat aattaagttt ctcttgtctt ggcatcaaga atagtcaagt |
| 1561 | tttttggccg ggcatggtgg ctcatgcctg taatcccagc acttggggag gccaaggcag |
| 1621 | gcgcatcaca tgaggccagg aattcgagac caacctggtc agcatggcaa aaccccgtct |
| 1681 | ctactaaaag tacaaaaatt agccaggcgt gatggcacgt gtctgtaatc ccagctactc |
| 1741 | tggagactga ggtgggagaa tcgcttgaga ctgggaggca gaggttgcag tgaaccgaga |
| 1801 | tcatgccacc gcacttcagc ctgggtgaca gagaaggact ccgtctcaaa aaaaaaagaa |
| 1861 | aaaagaatag tcatttttaa actacctatc tcatgcaatg aaagcatttt cttccacaaa |
| 1921 | gagcttaatc ctcatgatag gattgcctag tgtctcccat ttgcaggttt ctgggttgat |
| 1981 | gtcttaatgc ataatactgc aagtgacatc agctggctgt gatgcttcga aataggtctg |
| 2041 | ctcctcacag ctttgggaat ctgaatggaa gaagaaaaga gagaagttaa caacctccac |
| 2101 | tggggcaact ttgtgaacac gtaggcactt agtcatagga aacatattat gtgcaggtcc |
| 2161 | tagcctgggg gaggaaagta gatagacaga aaatcattag gtaatttaag tactaaattg |
| 2221 | ggcagggctt tttagtatca aatcactact agaccattta atttgttaaa ttatctctag |
| 2281 | gatggtgatt tataacctac ccaaagttat cgatattctt actaaactct gaggcctgaa |
| 2341 | gttctgtgat agaccttaaa taagtgtcct aagtcagtgg ttcccaaatc tggctggtcg |
| 2401 | ggaatacctg ggaagtttgt taaaattttt taaaaatgtt ttaagatttt tgggtcctga |
| 2461 | gccagccgtg gtggctcaca cctgtaatcc cagcactttg ggaggctgag gcaggtggat |
| 2521 | cgcctgaggt caggagttca agatcaacct ggccaacata ctgaaacccc gtctctacta |
| 2581 | aaaataagaa aaattagctg ggcgtggtgg cgggcacctg taatcccagc tacttgggag |
| 2641 | actgaggcag gagaatcgct tgaacctggg agttagaggt tgcagtgagc tgagatcaca |
| 2701 | ccattgcgct tcagcctggg caacaagagt gaaactccat ctccaaaaaa aaaaaacaaa |
| 2761 | aagaaaaaga tttttgggtc ccgacctcaa acctactgaa tcagaatttc tagggatgaa |
| 2821 | gcctaggaat gtgttgttgt tttcagagct tccctggtga ttgtgataag cctggtttgg |
| 2881 | aaaccattgc tggagaactt tgtaaagata cagagaccca gaccttttgt atttacattt |
| 2941 | aaatacaaat acaaatcctg ggtttctata tattctgtta gcttttcagg tgattctgct |
| 3001 | acacagacgt tgaaaaccac tgccctaaga aagagatcag aggccacata tcagagagaa |
| 3061 | aagggaccaa accttcggtg gtttgttgtg tgtcgtttta atgccaatta ttttaacttg |
| 3121 | cacagtcttc tgaaaccttg tattaatagt tctcttttgt attaccattt tcaggtaggg |
| 3181 | ttttgatcac tatgattctg aagataatag tgaaatagtg aatttcattg atatgaagag |
| 3241 | ataattgatt ttcattcatt ggtttgaaca cctgcaaaat cacaaataaa tgagaactaa |
| 3301 | gtcttgtaaa aaaaaaaaaa aaaa |

For example, the nucleotide sequence corresponding to the mRNA of the human RAB7L1 (transcript variant 2) is depicted in SEQ ID NO: 3 (3223 bp), wherein the underscored bolded “ATG” denotes the beginning of the open reading frame. Nucleotides 1130-1238, 1526-1779, 4045-4116, 4959-5140, 5758-5879, 6159-8626 of SEQ ID NO: 1 can be spliced together to form RAB7L1 (transcript variant 2). Sequence information related to RAB7L1 (transcript variant 2) is accessible in public databases by GenBank Accession number NM_001135662.1 (nucleotide).

SEQ ID NO: 3:

| | |
|---|---|
| 1 | agtgccacag gcaaccctgc acgtgacgct tgcggaggaa ggggagagag aggcgcgcgg |
| 61 | gagggcgtct agggaatcga ggtgccggct gctccttcct cacaatttgc ccagacaggc |
| 121 | ggtgggtggg aatgcctcac ttcagtttga agagggtccg gatccaaagg ggttaaaacg |
| 181 | agcgaccccc gatccccgac cacacttccc gcctccctaa aacgcacacc ccgctagcca |
| 241 | tgggcagccg cgaccacctg ttcaaagtgc tggtggtggg ggacgccgca gtgggcaaga |
| 301 | cgtcgctggt gcagcgatat tcccaggaca gcttcagcaa acactacaag tccacggtgg |
| 361 | gagtggattt tgctctgaag gttctccagt ggtctgacta cgagatagtg cggcttcagc |
| 421 | tgtgggatat tgcagggcag gagcgcttca cctctatgac acgattgtat tatcgggatg |
| 481 | cctctgcctg tgttattatg tttgacgtta ccastgccac taccttcagc aacagccaga |
| 541 | ggtggaaaca ggacctagac agcaagctca cactacccaa tggagagccg gtgccctgcc |
| 601 | tgctcttggc caacaagtgt gatctgtccc cttgggcagt gagccgggac cagattgacc |
| 661 | ggttcagtaa agagaacggt ttcacaggtt ggacagaaac atcagtcaag gagaacaaaa |
| 721 | atattaatga ggctatgaga gtcctcattg aaaagatgat gagaaattcc acagaagata |
| 781 | tcatgtcttt gtccacccaa ggggactaca tcaatctaca aaccaagtcc tccagctggt |
| 841 | cctgctgcta gtagtgtttg gcttattttc catcccagtt ctgggaggtc ttttaagtct |
| 901 | cttccctttg gttgcccacc tgacaatttt attaagtaca tttgaattgt ctcctgacta |
| 961 | ctgtccagta aggaggccca ttgtcactta gaaaagacac ctggaaccca tgtgcatttc |
| 1021 | tgcatctcct ggattagcct ttcacatgtt gctggctcac attagtgccagttagtgcct |
| 1081 | tcggtgtaag atcttctcat cagccctcaa tttgtgatcc ggaattttat gagaaggatt |
| 1141 | agaaatcagc acctgcgttt tagagatcat aattctcacc tacttctgag cttatttttc |
| 1201 | catttgatat tcattgatat catgacttcc aattgagagg aaaatgagat caaatgtcat |
| 1261 | ttcccaaatt tcttgtaggc cgttgtttca gattctttct gtcttggaat gtaaacatct |
| 1321 | gattctggaa tgcagaagga gggggtctgg gcatctgtgg atttttggct actagaagtg |
| 1381 | tcccagaagt cactgtattt ttgaaacttc taacgtcata attaagtttc tcttgtcttg |
| 1441 | gcatcaagaa tagtcaagtt ttttggccgg gcatggtggc tcatgcctgt aatcccagca |
| 1501 | cttggggagg ccaaggcagg cggatcacat gaggccagga attcgagacc aacctggtca |
| 1561 | gcatggcaaa accccgtctc tactaaaagt acaaaaatta gccaggcgtg atggcacgtg |
| 1621 | tctgtaatcc cagctactct ggagactgag gtgggagaat cgcttgagac tgggaggcag |
| 1681 | aggttgcagt gaaccgagat catgccaccg cacttcagcc tgggtgacag agaaggactc |
| 1741 | cgtctcaaaa aaaaaagaaa aaagaatagt catttttaaa ctacctatct catgcaatga |
| 1801 | aagcattttc ttccacaaag agcttaatcc tcatgatagg attgcctagt gtctcccatt |
| 1861 | tgcaggtttc tgggttgatg tcttaatgca taatactgca agtgacatca gctggctgtg |
| 1921 | atgcttcgaa ataggtctgc tcctcacagc tttgggaatc tgaatggaag aagaaaagag |
| 1981 | agaagttaac aacctccact ggggcaactt tgtgaacacg taggcactta gtcataggaa |
| 2041 | acatattatg tgcaggtcct agcctggggg aggaaagtag atagacagaa aatcattagg |
| 2101 | taatttaagt actaaattgg gcagggcttt ttagtatcaa atcactacta gaccatttaa |
| 2161 | tttgttaaat tatctctagg atggtgattt ataacctacc caaagttatc gatattctta |
| 2221 | ctaaactctg aggcctgaag ttctgtgata gaccttaaat aagtgtccta agtcagtggt |
| 2281 | tcccaaatct ggctggtcgg gaatacctgg gaagtttgtt aaaatttttt aaaaatgttt |
| 2341 | taagattttt gggtcctgag ccagccgtgg tggctcacac ctgtaatccc agcactttgg |
| 2401 | gaggctgagg caggtggatc gcctgaggtc aggagttcaa gatcaacctg gccaacatac |
| 2461 | tgaaaccccg tctctactaa aaataagaaa aattagctgg gcgtggtggc gggcacctgt |
| 2521 | aatcccagct acttgggaga ctgaggcagg agaatcgctt gaacctggga gttagaggtt |
| 2581 | gcagtgagct gagatcacac cattgcgctt cagcctgggc aacaagagtg aaactccatc |
| 2641 | tccaaaaaaa aaaaacaaaa agaaaaagat ttttgcgtcc cgacctcaaa cctactgaat |
| 2701 | cagaatttct agggatgaag cctaggaatg tgttgttgtt ttcagagctt ccctggtgat |
| 2761 | tgtgataagc ctggtttgga aaccattgct ggagaacttt gtaaagatac agagacccag |
| 2821 | accttttgta tttacattta aatacaaata caaatcctgg gtttctatat attctgttag |
| 2881 | cttttcaggt gattctgcta cacagacgtt gaaaaccact gccctaagaa agagatcaga |
| 2941 | ggccacatat cagagagaaa agggaccaaa ccttcggtgg tttgttgtgt gtcgttttaa |
| 3001 | tgccaattat tttaacttgc acagtcttct gaaaccttgt attaatagtt ctcttttgta |
| 3061 | ttaccatttt caggtagggt tttgatcact atgattctga agataatagt gaaatagtga |
| 3121 | atttcattga tatgaagaga taattgattt tcattcattg gtttgaacac ctgcaaaatc |
| 3181 | acaaataaat gagaactaag tcttgtaaaa aaaaaaaaaa aaaa |

For example, the nucleotide sequence corresponding to the mRNA of the human RAB7L1 (transcript variant 3) is depicted in SEQ ID NO: 4 (3438 bp), wherein the underscored bolded “ATG” denotes the beginning of the open reading frame. Nucleotides 1130-1779, 4959-5140, 5758-5879, 6159-8626 of SEQ ID NO: 1 can be spliced together to form RAB7L1 (transcript variant 3). Sequence information related to RAB7L1 (transcript variant 3) is accessible in public databases by GenBank Accession number NM_001135663.1 (nucleotide).

SEQ ID NO: 4:

| | |
|---|---|
| 1 | agtgccacag gcaaccctgc acgtgacgct tgcggaggaa ggggagagag aggcgcgcgg |
| 61 | gagggcgtct agggaatcga ggtgccggct gctccttcct cacaatttgg tttgtgctgc |
| 121 | aaggggaggg tccccatcat ctggccccag tggtgtaagg agctgactgg gattcagtca |
| 181 | ctgacttgga gccgctcggg ggaagtcccg gtgggtgagg ttccgcggcg cctggtccag |
| 241 | tttctcggca gtcaggccag gagggggtgg ggaaggtgcg aacagccggg ggctcggagc |
| 301 | tcgcgccctg cgccccaccc aggttgcggc cgcccgggga ggaagcgcgt ataggttgag |
| 361 | tgcaaagttt ccttttttgc tttctcgtca gagcagccca gacaggcggt gggtgggaat |
| 421 | gcctcacttc agtttgaaga gggtccggat ccaaaggggt taaaacgagc gacccccgat |
| 481 | ccccgaccac acttcccgcc tccctaaaac gcacaccccg ctagccatgg gcagccgcga |
| 541 | ccacctgttc aaagtgctgg tggtggggga cgccgcagtg ggcaagacgt cgctggtgca |
| 601 | gcgatattcc caggacagct tcagcaaaca ctacaagtcc acggtgggag ggcaggagcg |
| 661 | cttcacctct atgacacgat tgtattatcg ggatgcctct gcctgtgtta ttatgtttga |
| 721 | cgttaccaat gccactacct tcagcaacag ccagaggtgg aaacaggacc tagacagcaa |
| 781 | gctcacacta cccaatggag agccggtgcc ctgcctgctc ttggccaaca agtgtgatct |
| 841 | gtccccttgg gcagtgagcc gggaccagat tgaccggttc agtaaagaga acggtttcac |
| 901 | aggttggaca gaaacatcag tcaaggagaa caaaaatatt aatgaggcta tgagagtcct |
| 961 | cattgaaaag atgatgagaa attccacaga agatatcatg tctttgtcca cccaagggga |
| 1021 | ctacatcaat ctacaaacca agtcctccag ctggtcctgc tgctagtagt gtttggctta |
| 1081 | ttttccatcc cagttctggg aggtctttta agtctcttcc ctttggttgc ccacctgaca |
| 1141 | attttattaa gtacatttga attgtctcct gactactgtc cagtaaggag gcccattgtc |
| 1201 | acttagaaaa gacacctgga acccatgtgc atttctgcat ctcctggatt agcctttcac |
| 1261 | atgttgctgg ctcacattag tgccagttag tgccttcggt gtaagatctt ctcatcagcc |
| 1321 | ctcaatttgt gatccggaat tttatgagaa ggattagaaa tcagcacctg cgttttagag |
| 1381 | atcataattc tcacctactt ctgagcttat ttttccattt gatattcatt gatatcatga |
| 1441 | cttccaattg agaggaaaat gagatcaaat gtcatttccc aaatttcttg taggccgttg |
| 1501 | tttcagattc tttctgtctt ggaatgtaaa catctgattc tggaatgcag aaggaggggg |
| 1561 | tctgggcatc tgtggatttt tggctactag aagtgtccca gaagtcactg tatttttgaa |
| 1621 | acttctaacg tcataattaa gtttctcttg tcttggcatc aagaatagtc aagttttttg |
| 1681 | gccgggcatg gtggctcatg cctgtaatcc cagcacttgg ggaggccaag gcaggcggat |
| 1741 | cacatgaggc caggaattcg agaccaacct ggtcagcatg gcaaaacccc gtctctacta |
| 1801 | aaagtacaaa aattagccag gcgtgatggc acgtgtctgt aatcccagct actctggaga |
| 1861 | ctgaggtggg agaatcgctt gagactggga ggcagaggtt gcagtgaacc gagatcatgc |
| 1921 | caccgcactt cagcctgggt gacagagaag gactccgtct caaaaaaaaa agaaaaaaga |
| 1981 | atagtcattt ttaaactacc tatctcatgc aatgaaagca ttttcttcca caaagagctt |
| 2041 | aatcctcatg ataggattgc ctagtgtctc ccatttgcag gtttctgggt tgatgtctta |
| 2101 | atgcataata ctgcaagtga catcagctgg ctgtgatgct tcgaaatagg tctgctcctc |
| 2161 | acagctttgg gaatctgaat ggaagaagaa aagagagaag ttaacaacct ccactggggc |
| 2221 | aactttgtga acacgtaggc acttagtcat aggaaacata ttatgtgcag gtcctagcct |
| 2281 | gggggaggaa agtagataga cagaaaatca ttaggtaatt taagtactaa attgggcagg |
| 2341 | gctttttagt atcaaatcac tactagacca tttaatttgt taaattatct ctaggatggt |
| 2401 | gatttataac ctacccaaag ttatcgatat tcttactaaa ctctgaggcc tgaagttctg |
| 2461 | tgatagacct taaataagtg tcctaagtca gtggttccca aatctggctg gtcgggaata |
| 2521 | cctgggaagt ttgttaaaat tttttaaaaa tgttttaaga tttttgggtc ctgagccagc |
| 2581 | cgtggtggct cacacctgta atcccagcac tttgggaggc tgaggcaggt ggatcgcctg |
| 2641 | aggtcaggag ttcaagatca acctggccaa catactgaaa ccccgtctct actaaaaata |
| 2701 | agaaaaatta gctgggcgtg gtggcgggca cctgtaatcc cagctacttg ggagactgag |
| 2761 | gcaggagaat cgcttgaacc tgggagttag aggttgcagt gagctgagat cacaccattg |
| 2821 | cgcttcagcc tgggcaacaa gagtgaaact ccatctccaa aaaaaaaaaa caaaaagaaa |
| 2881 | aagatttttg ggtcccgacc tcaaacctac tgaatcagaa tttctaggga tgaagcctag |
| 2941 | gaatgtgttg ttgttttcag agcttccctg gtgattgtga taagcctggt ttggaaacca |
| 3001 | ttgctggaga actttgtaaa gatacagaga cccagacctt ttgtatttac atttaaatac |
| 3061 | aaatacaaat cctgggtttc tatatattct gttagctttt caggtgattc tgctacacag |
| 3121 | acgttgaaaa ccactgccct aagaaagaga tcagaggcca catatcagag agaaaaggga |
| 3181 | ccaaaccttc ggtggtttgt tgtgtgtcgt tttaatgcca attattttaa cttgcacagt |
| 3241 | cttctgaaac cttgtattaa tagttctctt ttgtattacc attttcaggt agggttttga |
| 3301 | tcactatgat tctgaagata atagtgaaat agtgaatttc attgatatga agagataatt |
| 3361 | gattttcatt cattggtttg aacacctgca aaatcacaaa taaatgagaa ctaagtcttg |
| 3421 | taaaaaaaaa aaaaaaaa |

For example, the nucleotide sequence corresponding to the mRNA of the human RAB7L1 (transcript variant 4) is depicted in SEQ ID NO: 5 (3070 bp), wherein the underscored bolded “ATG” denotes the beginning of the open reading frame. Nucleotides 1130-1339, 4045-4116, 4959-5140, 5758-5879, 6159-8626 of SEQ ID NO: 1 can be spliced together to form RAB7L1 (transcript variant 4). Sequence information related to RAB7L1 (transcript variant 4) is accessible in public databases by GenBank Accession number NM_001135664.1 (nucleotide).

SEQ ID NO: 5:

| | |
|---|---|
| 1 | agtgccacag gcaaccctgc acgtgacgct tgcggaggaa ggggagagag aggcgcgcgg |
| 61 | gagggcgtct agggaatcga ggtgccggct gctccttcct cacaatttgg tttgtgctgc |
| 121 | aaggggaggc tccccatcat ctggccccag tggtgtaagg agctgactgg gattcagtca |
| 181 | ctgacttgga gccgctcggg ggaagtcccg tggattttgc tctgaaggtt ctccagtggt |
| 241 | ctgactacga gatagtgcgg cttcagctgt gggatattgc agggcaggag cgcttcacct |
| 301 | ctatgacacg attgtattat cgggatgcct ctgcctgtgt tattatgttt gacgttacca |
| 361 | atgccactac cttcagcaac agccagaggt ggaaacagga cctagacagc aagctcacac |
| 421 | tacccaatgg agagccggtg ccctgcctgc tcttggccaa caagtgtgat ctgtcccctt |
| 481 | gggcagtgag ccgggaccag attgaccggt tcagtaaaga gaacggtttc acaggttgga |
| 541 | cagaaacatc agtcaaggag aacaaaaata ttaatgaggc tatgagagtc ctcattgaaa |
| 601 | agatgatgag aaattccaca gaagatatca tgtctttgtc cacccaaggg gactacatca |
| 661 | atctacaaac caagtcctcc agctggtcct gctgctagta gtgtttggct tattttccat |
| 721 | cccagttctg ggaggtcttt taagtctctt ccctttggtt gcccacctga caattttatt |
| 781 | aagtacattt gaattgtctc ctgactactg tccagtaagg aggcccattg tcacttagaa |
| 841 | aagacacctg gaacccatgt gcatttctgc atctcctgga ttagcctttc acatgttgct |
| 901 | ggctcacatt agtgccagtt agtgccttcg gtgtaagatc ttctcatcag ccctcaattt |
| 961 | gtgatccgga actttatgag aaggattaga aatcagcacc tgcgttttag agatcataat |
| 1021 | tctcacctac ttctgagctt atttttccat ttgatattca ttgatatcat gacttccaat |
| 1081 | tgagaggaaa atgagatcaa atgtcatttc ccaaatttct tgtaggccgt tgtttcagat |
| 1141 | tctttctgtc ttggaatgta aacatctgat tctggaatgc agaaggaggg ggtctgggca |
| 1201 | tctgtggatt tttggctact agaagtgtcc cagaagtcac tgtatttttg aaacttctaa |
| 1261 | cgtcataatt aagtttctct tgtcttggca tcaagaatag tcaagttttt tggccgggca |
| 1321 | tggtggctca tgcctgtaat cccagcactt ggggaggcca aggcaggcgg atcacatgag |
| 1381 | gccaggaatt cgagaccaac ctggtcagca tggcaaaacc ccgtctctac taaaagtaca |
| 1441 | aaaattagcc aggcgtgatg gcacgtgtct gtaatcccag ctactctgga gactgaggtg |
| 1501 | ggagaatcgc ttgagactgg gaggcagagg ttgcagtgaa ccgagatcat gccaccgcac |
| 1561 | ttcagcctgg gtgacagaga aggactccgt ctcaaaaaaa aaagaaaaaa gaatagtcat |
| 1621 | ttttaaacta cctatctcat gcaatgaaag cattttcttc cacaaagagc ttaatcctca |
| 1681 | tgataggatt gcctagtgtc tcccatttgc aggtttctgg gttgatgtct taatgcataa |
| 1741 | tactgcaagt gacatcagct ggctgtgatg cttcgaaata ggtctgctcc tcacagcttt |
| 1801 | gggaatctga atggaagaag aaaagagaga agttaacaac ctccactggg gcaactttgt |
| 1861 | gaacacgtag gcacttagtc ataggaaaca tattatgtgc aggtcctagc ctgggggagg |
| 1921 | aaagtagata gacagaaaat cattaggtaa tttaagtact aaattgggca gggcttttta |
| 1981 | gtatcaaatc actactagac catttaattt gttaaattat ctctaggatg gtgatttata |
| 2041 | acctacccaa agttatcgat attcttacta aactctgagg cctgaagttc tgtgatagac |
| 2101 | cttaaataag tgtcctaagt cagtggttcc caaatctggc tggtcgggaa tacctgggaa |
| 2161 | gtttgttaaa attttttaaa aatgttttaa gatttttggg tcctgagcca gccgtggtgg |
| 2221 | ctcacacctg taatcccagc actttgggag gctgaggcag gtggatcgcc tgaggtcagg |
| 2281 | agttcaagat caacctggcc aacatactga aaccccgtct ctactaaaaa taagaaaaat |
| 2341 | tagctgggcg tggtggcggg cacctgtaat cccagctact tgggagactg aggcaggaga |
| 2401 | atcgcttgaa cctgggagtt agaggttgca gtgagctgag atcacaccat tgcgcttcag |
| 2461 | cctgggcaac aagagtgaaa ctccatctcc aaaaaaaaaa aacaaaaaga aaaagatttt |
| 2521 | tgggtcccga cctcaaacct actgaatcag aatttctagg gatgaagcct aggaatgtgt |
| 2581 | tgttgttttc agagcttccc tggtgattgt gataagcctg gtttggaaac cattgctgga |
| 2641 | gaactttgta aagatacaga gacccagacc ttttgtattt acatttaaat acaaatacaa |
| 2701 | atcctgggtt tctatatatt ctgttagctt ttcaggtgat tctgctacac agacgttgaa |
| 2761 | aaccactgcc ctaagaaaga gatcagaggc cacatatcag agagaaaagg gaccaaacct |
| 2821 | tcggtggttt gttgtgtgtc gttttaatgc caattatttt aacttgcaca gtcttctgaa |
| 2881 | accttgtatt aatagttctc ttttgtatta ccattttcag gtagggtttt gatcactatg |
| 2941 | attctgaaga taatagtgaa atagtgaatt tcattgatat gaagagataa ttgattttca |
| 3001 | ttcattggtt tgaacacctg caaaatcaca aataaatgag aactaagtct tgtaaaaaaa |
| 3061 | aaaaaaaaaa |

For example, other mRNA transcript variants of human RAB7L1 can exist. For example nucleotides 1130-1238, 4045-4116, 4959-5140, 5758-5879, 6159-8626 of SEQ ID NO: 1 can be spliced together to form RAB7L1 (transcript variant 5). Sequence information related to RAB7L1 transcript variants is accessible in public databases such as GenBank.

For example, the polypeptide sequence corresponding to human RAB7L1 (isoform 1) is encoded by the nucleic acid sequence of SEQ ID NOS: 2 or 3 and is depicted in SEQ ID NO: 6 (203aa). Sequence information related to RAB7L1 (isoform 1) is accessible in public databases by GenBank Accession numbers NP_003920.1 and NP_001129134.1 (protein).

SEQ ID NO: 6:

| | |
|---|---|
| 1 | MGSRDHLFKV LVVGDAAVGK TSLVQRYSQD SFSKHYKSTV GVDFALKVLQ WSDYEIVRLQ |
| 61 | LWDIAGQERF TSMTRLYYRD ASACVIMFDV TNATTFSNSQ RWKQDLDSKL TLPNGEPVPC |
| 121 | LLLANKCDLS PWAVSRDQID RFSKENGFTG WTETSVKENK NINEAMRVLI EKMMRNSTED |
| 181 | IMSLSTQGDY INLQTKSSSW SCC |

For example, the polypeptide sequence corresponding to human RAB7L1 (isoform 2) is encoded by the nucleic acid sequence of SEQ ID NO: 4 and is depicted in SEQ ID NO: 7 (179aa). Sequence information related to RAB7L1 (isoform 2) is accessible in public databases by GenBank Accession numbers NP_001123135.1 (protein).

SEQ ID NO: 7:

| | |
|---|---|
| 1 | MGSRDHLFKV LVVGDAAVGK TSLVQRYSQD SFSKHYKSTV GGQERFTSMT PLYYRDASAC |
| 61 | VIMFDVTNAT TFSNSQRWKQ DLDSKLTLPN GEPVPCLLLA NKCDLSPWAV SRDQIDRFSK |
| 121 | ENGFTGWTET SVKENKNINE AMRVLIEKMM RNSTEDIMSL STQGDYINLQ TKSSSWSCC |

For example, the polypeptide sequence corresponding to human RAB7L1 (isoform 3) is encoded by the nucleic acid sequence of SEQ ID NO: 5 and is depicted in SEQ ID NO: 8 (131 aa). Sequence information related to RAB7L1 (isoform 3) is accessible in public databases by GenBank Accession numbers NP_001129136.1 (protein).

SEQ ID NO: 8:

| | |
|---|---|
| 1 | MTRLYYRDAS ACVIMFDVTN ATTFSNSQRW KQDLDSKLTL PNGEPVPCLL LANKCDLSPW |
| 61 | AVSRDQIDRF SKENGFTGWT ETSVKENKNI NEAMRVLIEK MMRNSTEDIM SLSTQGDYIN |
| 121 | LQTKSSSWSC C |

The invention provides for a nucleic acid encoding a LRRK2 protein, or fragment thereof.

For example, the human genomic nucleotide sequence corresponding to the sense strand of the human LRRK2 gene is depicted in SEQ ID NO: 9 (144275 bp). Sequence information related to LRRK2 is accessible in public databases by GenBank Accession number NG_011709.1 (nucleotide).

SEQ ID NO: 9:

| | |
|---|---|
| 1 | gcgctggctg cgggcggtga gctgagctcg cccccgggga gctgtggccg gcgcccctgc |
| 61 | cggttccctg agcagcggac gttcatgctg ggagggcggc gggttggaag caggtgccac |
| 121 | catggctagt ggcagctgtc aggggtgcga agaggacgag gaaactctga agaagttgat |
| 181 | agtcaggctg aacaatgtcc aggaaggaaa acagatagaa acgctggtcc aaatcctgga |
| 241 | ggatctgctg gtgttcacgt actccgagcg cggtaatcac ttgaaaataa actgtgcttt |
| 301 | tatttttgca aactttctcc ccctccttac atttgcaaat tttgtcctcc tccccttgac |
| 361 | cctgctcaaa cccggactct taaggagccg caaactccca tatcctttcc ttagggcaga |
| 421 | aagcagctga gaatttcagg aaggtcttca cctttttgac ttttctcccc gtttcagact |
| 481 | aaaaaggaga gggggtgctg tggattgtga ctttgcttct tttccccacc cacttgtttt |
| 541 | ccagcctcca agttatttca aggcaaaaat atccatgtgc ctctgttgat cgtcttggac |
| 601 | tcctatatga gagtcgcgag tgtgcagcag gtaaaggcat tgttttcact tcaactcatt |
| 661 | ctcccttctg tttggaagga gacgttttac tggcaatgtt aatatagccg agagttcttg |
| 721 | gttattccca aaatttggct tgaggaacct ctgactgtga ttttaagatg ggaatattgt |
| 781 | taaatcatta cgcaatgtaa acgggatgaa gagccccagt atgtgttccc tgagtgtctt |
| 841 | taagaagtaa ctttataaaa ccaacagtat ggatggtggt agaaggagga taaaaatggg |
| 901 | ttcggtttta gtctcgttat tggcaagatg aattcattag tgtttagact tgactattcc |
| 961 | aagtatcttc ccaatacaga gcatgtccta gatgagaaga ttatgaatag tttggaaaag |
| 1021 | gggaataatt aatagtgata aaatgcaact ttgtcactag caaactcttg tagagttcag |
| 1081 | cactttttaa aattcaaaga tttctagcct ttagttgtag tataccttgt agtatctaaa |
| 1141 | gaaagtgatg tcttatgaga ccctcatagt ttgcaactgt tgtcatataa aatgcatgta |
| 1201 | gaagtgaaac ttttacaatc tgtaccatag gaaacccaga aatttgctat gtatcttgga |
| 1261 | ttttttttta aagggggcct taaaaatggt aattaagaat gatttacagt caaaacaaaa |
| 1321 | ttataggcca aggtgataac ttccttcgga gcacttagag atttggggaa ctgaaatcag |
| 1381 | ttttgtcatc tgcatgttaa ctcatgcaga gaaagagaat tggactttga actccttgga |
| 1441 | ggtgcagtca gaaagccaat gtttcttaat ggttgagagg cttgacagac atgaggcatc |
| 1501 | tcaatcttta aagtggtgtg ggtctatctt tatcttgatg tttatctctg tatctagctg |
| 1561 | tatctagtct gggtgaacca tctagcttct ttgatatgag gacatttaca tctggaagaa |
| 1621 | atattttaat ttgttttcaa ctgtgaaata ttttccatct gactattata gattttcacg |
| 1681 | ctgctatcaa accaaaccaa gaaaagatgg aggcataata aagatgctgt tcttttaaga |
| 1741 | ctcaaagtcg gaattttgcc tgtggaatat gagtcacttt ttgggcactg gcctattgtg |
| 1801 | cttcctgctc tgcacccacg tcatcccttc ttacttgtct ctgctttggt gttcagaagt |
| 1861 | gcctgattct ggccaccttc attccctaga ctctgtactt gatagagtca ctcctgcttg |
| 1921 | atactgctca ggacagtcag atcctgggta ggcgttttgg tctgcagggt ctagataagg |
| 1981 | cagtgctata cttgacaacc caggggagcc tggaacatac ttcctaattc ttaattttag |
| 2041 | aaattgccca agcctgagca tacttgtccg gagtagttat gagtgtcact tagtatttct |
| 2101 | gcctagagag taccagaggc aaagtatgct ggaaaataag gaagagtttt tttaaaagta |
| 2161 | attaattact tttttggata tatcatagtt gtatatattt tggggataca tatgctattt |
| 2221 | gatacatgta tacaatgtgt aatgttcaaa tcagggtaac tggaatatcc gtcacctcga |
| 2281 | acatttttct ttgtgttggc aacgttgcaa tttctttctt ctagctattt taaaatatgc |
| 2341 | aatgaattat taaccataat ttccctgcta tactattaaa tattaactta attgcttgta |
| 2401 | tctaattata tttttgtaca cattaaccac cttctcttta tccctcccca tcctttcatt |
| 2461 | tccagtctct ggtaaccacc attctactct cttcctccat gagatccacc ttttccgctc |
| 2521 | ctacatatga gtgagattat gcaatatttt atttctgtac ctggcttatt taatttaacc |
| 2581 | taatgacctc cagtcccacc catgctgttg caaatgacag gatttcattt tttatgactg |
| 2641 | aataatattc cattgtgtat gtataccaca ttttctttta tttttagtta agtaattaat |
| 2701 | ttagagacag ggtctcactc tgttgcccag actggagagc agtggtgtga tcaaagctca |
| 2761 | ctgcaggcct gcaatcctgg gctgaagtag tcctcctgcc tcagcctccc aggtagctag |
| 2821 | gactataagc atgtgccacc atgctcagct aatttttttt tcttttttta ctttttgtag |
| 2881 | agatggagtc ttgctatgtt gcccaggtta gtttcaaact cctgacctca agtaatcctc |
| 2941 | ctgcctcggc ctccatattt tctttgttaa tctgttgata gacatataag gtgattctgt |
| 3001 | attttaacta ttgtgaacag ggctgcaata aacatgggag ttcagatata tctttgatat |
| 3061 | actgatgttc tttttttgga tatataccca gcgatgagat tactggatca tatgaaaatt |
| 3121 | ctctttttag ttttttaaga tacctccata ctgtgtttca tcatggctgt gctactttat |
| 3181 | attcccatca gcagtgtacc accattcccc tttttctgca tccttaccag catttgttat |
| 3241 | tttttgtctt tttgataata gccattctgc ctgtggtgag ataatatctc attatggttt |
| 3301 | tgatttgcct ctccctaatg attagtgaag tttaggattt ttttttcatg tacctgttag |
| 3361 | ccatttgtct gtcttctttt gagaaatgtc tatttggatc ttttgtccat ttaaaaataa |
| 3421 | gacttttttt tttttttttt tttttgctaa ttgagttttt ttagttcctt atgtattctg |
| 3481 | gttgttaatc ttttgttgaa tggatatttt gcaaatattt tctccctttc tttatgttgt |
| 3541 | ctcttcactt tgttaattat tttctttgtt gtgcagaagc tttttagctt gatataatcc |
| 3601 | catttgccta tttttgttgt aattgcctgt gcttttgagg tcctacccca aaaatcattg |
| 3661 | cacagaccaa tgtcctgtag catttcccca gtgttttctt ctagtagttg catattttca |
| 3721 | ggtctgagat gtaagtcttt aatccatttt gaaatgattt gtgtatatgg tgaaagctgt |
| 3781 | ggatctagtt tccttctttt gcacagccaa tatttgattc tcactgaaat ctcactgccc |
| 3841 | tcctggaaat attaccggat gttttactgt catagcgaag tgagagtaag ctgctcactg |
| 3901 | aggatcaaag agcttgtgac agacctaaga ctcaagtctt ctcacacctt caaaatctct |
| 3961 | ttccatcata caatctacta gctgctgaat tcgcaagctt tttgtgcaag ctagtaaaaa |
| 4021 | gcaaaatggt ttgatacaaa tactgtggcc atgctaggta caatgacatc aatttaaatt |
| 4081 | atcattggtc ttaacaaggg gatgtagaaa ggggtctcct actgacattt taatactcac |
| 4141 | ttaaaagtag tatttttcct tcagatttct ttatattatt agtataatta ctgtaagtat |
| 4201 | cctttactgc tttatatgtt gaattagctg gaagtgccaa aagaaaaact cttaatgata |
| 4261 | aatttaaggt attaaggtaa atttctcctt catttaattt aatagtaatt cttaattact |
| 4321 | atttaaaata aagattaagg tttgtttcta gatgccattt aacatgatat tccagactgc |
| 4381 | cagttttatt ttcaaagttt gtttcatatt ttattaatgt ttcttcataa atgacagtct |
| 4441 | ttagaaaatt gacggttaag ctaggtgctt tatatttttc ttttcctgcc tatcttttca |
| 4501 | ctgtgctcct aaattttaca tctctttatt ctcaagggtt caacctttga agaaggggag |
| 4561 | caaaataaat gaaagtggct aaaatttttt ctttaacccc tagactcttt cctgttgtgc |
| 4621 | attaattaca tgcttgagtt tttagaataa ttataataaa gtaaaactac caatttaatt |
| 4681 | gtattgtaac tgtgcaagat gggaaccttc tctcttagag agataagctt ttaattgaat |
| 4741 | agattaatgg atcaattgtt acctctgctt tgctgccaga gattctattt aatcacagaa |
| 4801 | gttccatgta gtgctggaga gctcagttgc ctgaatcttt ttgcaaagcg tttactgata |
| 4861 | ctgttgcttc accaaccaaa acaaacaggt tttttccttg agtcagcttt gtaggtacag |
| 4921 | agatgagttt ggcatcctat gtgacttttt tttttttttt tttttttttt tttaccacca |
| 4981 | gaagctgttc agaatgttat tttcttaaat agttcggaaa aaagtcttga tgtattctat |
| 5041 | gaaagcacaa aaatagtcag tttctatgac agctggattg tcaacgtctg ttcagcttac |
| 5101 | gtggaggagg atgtcctact tgagtagtat aggtagaaat agctatcaga aattgccgcc |
| 5161 | tttgaaagca atttgaaatt atgtaaaagg aagtaatgac aaaataaagc aatttatgtt |
| 5221 | taatctggaa aagatccaaa agtaatattg taaagagatc ttgagtaatc atttttatct |
| 5281 | tcctaaaata gccgttgttt actcccgtaa gcgagtaaga aacttgtgcc attattcctt |
| 5341 | attgggtgca tatagatttc tcaccttgtc attcaactcc ttgcaatatt caactttact |
| 5401 | tatgcatcca gccttatccc aaaatagcct cttccctgta gcagcttcct tatcatgtag |
| 5461 | cagcctactc tcctcaccgc tcatccgttc ttatacaatc tggcttaagc tctacctctt |
| 5521 | catattatat tcttctctga gtaatttcaa ctcacactga gtcttacttt cagtatttct |
| 5581 | attatattgg tatttattac acatcacact tagatacttt tccattagtc tctagagggt |
| 5641 | acatatatgc actttctctt tttttttttt tttttgacca tgtcaagtat agttctatag |
| 5701 | tataatagaa tgaattggag atcctttaca tttagagagg gaggagtcta cagtaggaag |
| 5761 | aataggttaa ttttcatctc cggtttgaaa tcaggacttt caattttttt ttcagaggta |
| 5821 | aagagcacct tagtcaagtt ggcatcttgt aaacagactg agtgaagata tacttaaaat |
| 5881 | gcatctataa tttcatattt tatttcgaaa tgtgaaagag cctactaggg gtgtctgtga |
| 5941 | tctctagacc ttatcaattc attctagaga aatctggagg gagccattga ggagttctac |
| 6001 | ctcctgtcta ttttatagag ctctttctct ttttctccta tcagatgtag attcaattgc |
| 6061 | taaaaatgcc acgtttcttg cctctattat tctagcttca ttacttgggg agcagccatt |
| 6121 | ctgataactt acattttgct actaaaatct ccaacttcac ctaatccttc attataagcc |
| 6181 | acttcatttt ttcctataat taaaatttta aatatgtgga ggaaattctg tcaggtagat |
| 6241 | atgacttaaa acctactaag ggccaggtgc agaggctcac gcctgtaatc ccagcacttt |
| 6301 | gagaggccaa gtcaggagga ttgcttgagc ccaggagctt gagaccagcc tgggcaacag |
| 6361 | agcgagaccc ctgtcttcac aaaaaaaaaa aaaaaaaaat tagctgggtg tggtggcaca |
| 6421 | tacctgtagt cccagctact tgggaggctg aagtgggtgg atcacctaag gacaggagtt |
| 6481 | ctaggctgca gtgatctatg attgcaccat tgcactccag cctgggtgac agagtgagac |
| 6541 | cctatctcaa aaacaaaaac aaaaccaaaa aaaaaaaaaa aacaagaaaa aaaaagtact |
| 6601 | aaggatctgg tatagccatt cttgcactta ataatcttgg tacaacctct aaaactattt |
| 6661 | tttatagttt attttatttt gccttattta gacaattggc atgtctatgt tcttcataat |
| 6721 | ttagaaatta tatgtattta tatatataaa ttatatatat tatatattat gtataaatta |
| 6781 | aattatatat atatataata tatatatatt tgaaatggat tcttgctctg tcatccaggc |
| 6841 | tagagtgcag tggcacaatc ttggctcact gcaccctctg cctcctgggt tcaagcgatt |
| 6901 | ctccttcctc agcctcccga gtagctgaga ttacaggcgc ccatcaccat gcctggctaa |
| 6961 | tttttgtatt tttagtagag actgggtttc accatcttgg ccacattggt ctcaaactgc |
| 7021 | cggctgaaat gttgtatttt ttgtatgtct ttctggtatg atttttggag aaaggtgtat |
| 7081 | cctaagaata cggcttgctt ttgtttctgg gtaagcattt tagggtatca ttttgttgta |
| 7141 | taaccattgt ttacaagtga gataagcatc tattccacta agattgaaga gattcatgtt |
| 7201 | tgactgagta tgctctatta acattcttta aaacatgtga atatatgtct ttcttgtttt |
| 7261 | caggtgggtt ggtcacttct gtgcaaatta atagaagtct gtccaggtac aatgcaaagc |
| 7321 | ttaatgggac cccaggatgt tggaaatgat tgggaagtcc ttggtgttca ccagtaagta |
| 7381 | tgatagatat gtaaaacaaa tggccttgag tatttatttg tacacatgac aaccttccct |
| 7441 | tgatacactg tgtttgcaat ccaaggctac tcctgtggaa ttctttaaaa tacagatatt |
| 7501 | tttccttgag tcaatgattt acatttatag agagctttaa actcagaagt ttgatttaga |
| 7561 | aagcaaacat ttaaggtaac atgtcagaag ttattatttt aataatataa tcatataatt |
| 7621 | ataaaactgg ttaagttgta gatttttgat gagtactttt gaattcaaac catgaagaga |
| 7681 | ttttggcttt taataataga atcgatacaa accactagtt cttaaaaaaa tgggaactga |
| 7741 | gaaaagttag ttctgtaagt agtaatttga aagttgatgt tctactgtct ttaaatagta |
| 7801 | catttatata tatattccta tatatacagt aagtttaaac tatggctttc agaaagagtt |
| 7861 | aagaaagagg aaattaactt tcagcacatc tgtagccaaa tcttgatagt aattttacca |
| 7921 | gctatgtttt tgcagtttgc agcataatgg cttcttagat gagactactt ccttagccat |
| 7981 | cattaattaa gaaaatattt tctcaagaag aatgtgtttc caggaaaata cattttggat |
| 8041 | agctttgttt cttgacagtt aaaaaatatc ttctaagcta ctgaggaggc tgaggaagga |
| 8101 | gaatcacttg aacctgggag gcggaggttg cagtgagcgg aggttgcagt gagccgagat |
| 8161 | tgcaccactg cactccagcc tgggtgacag agcgacactc tgtctcaaaa acaaacaaac |
| 8221 | aaacaaacct tttgtcatta actttaaatc ttttttatac ctaatatgac ttttctttat |
| 8281 | cacagaaaag gaaattgtga atattttttg gcttccaatg gtatatggtt tatgaaaatt |
| 8341 | taatttatga aaaattttca ggtgtttgta ttgctgatca gtgtcaagta gtgctataaa |
| 8401 | tttagacaaa ttagagctat gtgtttgtcc ataagtgaac atgtctgtgc ttatacattt |
| 8461 | tcccctcttt gacaaatgtg ttgctcttct tgttttcagt acataaaggg tgtgttttgg |
| 8521 | aaagagcata tttacaatta attggagttc tcgtcttcaa tctaatctct gtaattctat |
| 8581 | gtatcagttc taaagtatac agcatttgat gaggaattac tcaaaatata ccagtaatta |
| 8641 | ggaattgtaa ctttaaatgt cccttggttt gggtgataat ttccaggaag tccaaagatg |
| 8701 | agccagtcta taacctcagg gagtgtttgg gaaactcatc tagtcatatt cctgtacaaa |
| 8761 | ccaactgttc aaattaaatt acataaaagt ttatgtagga aatttcattc actcactcac |
| 8821 | ttactcattc actcactttg ttcatccagt cattcatctt ctattcattg aatgtttttg |
| 8881 | aagcctgtcc tctgggtcag aaaccatgca gttgtgaaga agatagacac actgctgtct |
| 8941 | ccagtggagt gtattagatc actcccagca aaaattgatt gtaaaacaga tttctctttt |
| 9001 | ttcaaggcct tttccctcca aagacttacc agtactgaag aaaaatttct tccgtggtaa |
| 9061 | taaagtcagg aattgtggga atggtatagg gagaggtagg ggcagggtga ttaggaggaa |
| 9121 | ggctggcaga gaatcgaaga ctggcttcat tcaggtcctc caattgccaa atggagatta |
| 9181 | tgcaacgttt cttgaataca tacaaaactc tagatgtggc cagctcagtc ttcttccaat |
| 9241 | aatgtaaagc caaacaatgc tttgcaggaa tagactagag attatatttt gggattaata |
| 9301 | acatagggat taaaatctta tcttgaacta actaaacatt attgatatgc taaattcact |
| 9361 | tttttttttt tttttttttt ttttttttag acagagtctt tctctgttgc caggctggcg |
| 9421 | tgcagtggcg tgatctcggc tcactgtaac ctctgtgtcc cgggttcaag tgattctcct |
| 9481 | gcctcagcct ctcgagtagc tgggactgca ggagtacgcc accacgccca gctaattttt |
| 9541 | gtatttttag tagagacagg gtttcatcat gttggccagg atggtcttga tctattgacc |
| 9601 | tcatgatccc cccgcctcgg cgtcccaaag tgctgagatt acaggcgtga gcaacggcca |
| 9661 | ccggcccact actttttaat atatcattaa tttctctttt aaaaacagta gcaatcaata |
| 9721 | atttaaatat tcaaatgaat tcttaattta tatacacaaa ctaacatctt tattatatct |
| 9781 | ctatatttta atatatcaac atgtctaaga taatttataa atttacatca tatataaaaa |
| 9841 | tgggtttgct ctcgatgtat ataaggcttc atgatatttt gaatatggag ttgggtgaaa |
| 9901 | atagtgaatc tgaatatttg aatttgaata tttattggaa aataagtagt gcttttaact |
| 9961 | ttttaaatga gacacataat agtcccctgt tgattttttt ttattttttt aactttatta |
| 10021 | aagtatagtt gacaattaaa aattgtttat atttaaggta tacaattgat gatttgacat |
| 10081 | gtgtacattg tgaattattc accacaatca agctaattaa cattccctgt tagtttttat |
| 10141 | aagcctggtt caggtttgta gaaagaaaca aacacacatg gccaggcacg gtggctcaca |
| 10201 | cctataatcc cagactttgg gaggccaagg caggaggatc acttgaaccc agaagtttag |
| 10261 | accagcctag gcaaaatagc aaaccctgtc tctccaaaaa agaatgaaaa aattagcctg |
| 10321 | gtgtggtggc atgtacctgt atccctagca actcaggagg ctgaggcagg aggattgctc |
| 10381 | acttgagccc aggagtttga ggtttcagtg agctatgatt gcaccattgc attccagcct |
| 10441 | gggtgacaca gcaagaccct gtctctaaaa gcaggcaaca aaaacacatg agcttcacta |
| 10501 | cagggaatta aatacaatga gagtaataaa aaataggtga gcaaaaaaaatgcaaaataag |
| 10561 | caaacttttg agtatgatat ttcattctta tcttgatttc tgtttttaac tccagattga |
| 10621 | ttcttaaaat gctaacagtt cataatgcca gtgtaaactt gtcagtgatt ggactgaaga |
| 10681 | ccttagatct cctcctaact tcaggtaata tgtgtatatg ttttttgtgt tgattcaaat |
| 10741 | taaaaaaaaa gttgatacca ttaagtaaat gtgtgtgtgt gtgttttttt tttttttttt |
| 10801 | ttgaagatca ggattagggt agcttgattt aaatgtccta aaattgcatc tgtttttaga |
| 10861 | cctagtgatg ggacagccat aatataatct aaatatcagt tatttccaaa attctttctg |
| 10921 | tttccatctc ttctccttat ctcttttctc tatactttgc ctctcaaaaa tctcattcaa |
| 10981 | tacattggtt ttaaacatta ccttatatat tatccccaaa tctctgttgg tagtccgatg |
| 11041 | tttgctccca aaatctggac ctacatctca tattccccca ggttagtggt cattcctgcc |
| 11101 | cttgccatta ttacttcttt ctccctatat atctttaata aattttctat aatatatgtt |
| 11161 | ctggagtatg ccataatcgt tacattttga aaacacatag tattacttct tgagtatttg |
| 11221 | ctagatgcca ggcttcacaa gtaaaatgct tcacgtgctt ttaaacacct gaatctgaaa |
| 11281 | acaccccttg agatagggat tttatctcag ttttctaagt gacaaacact gaagtgcaga |
| 11341 | gaagttgctt tggccactaa gaggtagaaa cagggtctga ttctccatgt caggttcctt |
| 11401 | ccctgagaaa actttggcct ggtagataat ggacctgaaa acaaaaaatc ttgaaatgat |
| 11461 | gcaacagttg tgggcattgc tgtgctggac actggctatt atataaggtt ccgagaagaa |
| 11521 | aggccgctca cagggagcta atcttgaagg gctgggagga gtttccttcc atgtagggga |
| 11581 | gggcttttta ggttgagaga agtatgggtg cagaggcctg ggaggatagc atgagagagg |
| 11641 | ctggacgtgt gattgggaag gtttgaattg tcctcatcag tcctgctaag agatgtaaag |
| 11701 | accatgctgg agaaagagga agatgagagt atgagggaaa aacaagaagg tactcaacat |
| 11761 | ttcactacag ctttttatga ccatgttgta tggcatgcac taagagtctt taaccatgat |
| 11821 | ttaatttaac ctcacccttg ggaggtattt tttttttgac ggagattcat tctccatttg |
| 11881 | gcgatggaca ggaagatgag ggtttattaa tatgaaaaat ctaccaacac tggaatatat |
| 11941 | tgaagttagc ctcatacagt actactactc ctattccagt attattattt ttattgacag |
| 12001 | aatagatgct gtttgtgtta agttttggat tatgatagga aatgtttggt atagtaaaag |
| 12061 | gcaagagtgt gacatgcagt tagtccaagt acgaagagat accaaaaaaa aaatgtttag |
| 12121 | tgaggagcag agtttagcat atttggagtg aagacaatgt gcggaaggaa aggagctgat |
| 12181 | gagatacatg tactatagtc ggttgtgtga aaggtcttgt ttttcatact aaggatcatg |
| 12241 | agaagatctt agtagattcc agcaagggat ttgcaagacc acatttgtgt tttagaaata |
| 12301 | taatgcaggc aataaaccag ttggatagaa attggggact gtagagcaat taagcaactg |
| 12361 | tttttgcatt ctagatgaga atgcaaaaca caataggaat gaaggtgcct tgttcaaaag |
| 12421 | gagttttgtt caaaaggaat cttcaagatg tgtaggagat attcttaaag gacttggtaa |
| 12481 | tgaattgatt tgttgcttgg atagagaatg agaagagaaa ggagggtgga agaaggaaga |
| 12541 | tgacttagga gtttctcttg ggtagctagt ggattatggt atcattgatg aagacaggga |
| 12601 | acaggagtag gccaggtttg gggtaactgt ggaatattca gatgttgtct aagaggcata |
| 12661 | agaatgtatt tcagatgttt ggggcaagtt gtctaggcta gaagtactga ttgagactca |
| 12721 | tgaaattata gtaaagtgaa ctgggagttt atctcattta taaagatcta gagcttgata |
| 12781 | agtctaacat ctagggcagt taagtagttt atcaacaaac aaacaaacaa acaaacaaga |
| 12841 | aaaccatggg tctacaaacc attcacagtc ttcatgtaaa aattaattca tgtaaaaatt |
| 12901 | aacacattaa atgttaaagc agctctttac tcagagcata ttattctctt taaaataggt |
| 12961 | aaaatcacct tgctgatatt ggatgaagaa agtgatattt tcatgttaat ttttgatgcc |
| 13021 | atgcactcat ttccagccaa tgatgaagtc cagaaacttg gatgcaaagc tttacatgtg |
| 13081 | ctgtttgaga gaggtatttt aaaatgtcaa attccttaaa gtatatataa gaaaaaaagg |
| 13141 | cttatactgg gaaaagtaga acacagttat aataagaaga aggtttctaa aatcctacta |
| 13201 | tttattaaga agtgggagtt gtctgtcaag ggtgaggaat ggggttaatt cagaagtatt |
| 13261 | gcttgttttg gtggggtgaa tttcattcgt gggttataaa tcatgcccct ggagtagact |
| 13321 | ttcttcaatt gcttaacaag gcataaggtt tactttgaaa actggatgtg tgggtgctat |
| 13381 | gaaagaaaaa ataaaactgt gaagccaagc ataggttaca ctgggattat gatgttgagt |
| 13441 | catcaccaga aatcatagaa attgcataaa gagcctgaag gtttacaaag tgtccttcag |
| 13501 | gaaaaagact aatatgcatt tcatagcctg gccctgagat tgataactga gattattatg |
| 13561 | taattttaga gttggttgga gtccttgttt agtctttcca ttgaccttag gaggaagtgg |
| 13621 | gtcacagcag tgaagtgagc atcctgcctg aggacacaga gcttgtgaca gtacagttca |
| 13681 | attagcaatt attttaagag ccccttttgt atcattatga gagccaactg tgctaggggt |
| 13741 | ttagataaga atgatttatg tgggccctgt gtcagttatc agtttaccag tctaatttct |
| 13801 | tgcagttccc agaatgggat agatcacctg ataactgttg aattccctgt ctcctcccag |
| 13861 | aaggatttta aacagcttat agataattat aatacacaag agtaaacaaa atggatgaga |
| 13921 | aaataggtga agggacaata atataaagct agattaagtt tactgtgttt ctaaggtcct |
| 13981 | gcatatttac aaaggggtgg gccagaaatt tgtctgtttg cttcctatct gacaaagaaa |
| 14041 | agaggtaaat atcagtggtt acaaagttcc ttaagataaa agtaaaccta ttattcagga |
| 14101 | gaagcaattg gtctcatggg agatctgaga aacatcttcc catgggtttt cctggatgag |
| 14161 | acaataaagg acatacattt tgcaaggaat acaaagtgta ttgcagcgag agtgactctg |
| 14221 | tcaaaagtca gaatagcatg ggcctggtac ccagctcttt gataatcata caccgtgaag |
| 14281 | tagaagatag tttacagcga gtacggaatt ccttcaggct gtcatgtata aatgttctat |
| 14341 | cttgcaacta agctttcgat gacaattagg ataaagtttg aggttctatt gtcttgcagg |
| 14401 | gtctgtaatc ttctgtgtgg aaggttaggg gcacattctt cttcctggaa ggagggctag |
| 14461 | catcacttta tcaccatcgt tgtttagtcc atctaagaca ctggaggtag accatagaat |
| 14521 | gttacaaaga agaatgttgc tcaatagaaa aaccatcagt gctgagaggg ttatgactat |
| 14581 | aaatgtagag tagaaaaatt tctgattttt ccaggagtat caggttctcc aggactcagg |
| 14641 | ggtgactata aagttaattt tcaaaatttg aaagtgtact gtggaaacta gaccataaag |
| 14701 | tgagaaagtt ccatgatatt cttcacttgt taggaaaact taactgattt cacattatat |
| 14761 | tatagggaca ctctggcata aaattaaaaa aaatgaatgt tgatcactta gagtgctgtg |
| 14821 | ttttctaaca tatttctggc gccattctca agctagataa actatazttt tatacatgtt |
| 14881 | tttcaggttg ttgcccaata acaatgactc caaatggaac ttactggctt gatcaaatga |
| 14941 | ctttaattgt gaaaattaat gatttatatt tttgctgtct gatggaaaac cactaagaca |
| 15001 | gagtatttca aagtctgatt acttgccatt tgctcaagtt gacaactctt gaactgaaac |
| 15061 | atttagccga gctgcccttc agcagcctac cattaatgcc tcccttttaa atattgcaat |
| 15121 | atgtccagtt ccagttggcc atctttatta gtcactgtca gttttctcta gaatttccca |
| 15181 | aatgaaattg taaataattt tgtttttctg agaactgctt gctgactagc acttttacat |
| 15241 | ttcaaaacat ggagtaccta acataggccg aaacaaaatt atttgaatct ccgtagcttg |
| 15301 | ttttctcatt ataacattct taggaagggc tgcttcacag aaatatattt tttatttaag |
| 15361 | gagattacac ttgatgtatc tcacacaact ataatgaata ttgtaatttt tgaataatta |
| 15421 | aactttcata tcatctttaa gcttattcag tattttgtct ttcattttta agtctcagag |
| 15481 | gagcaactga ctgaatttgt tgagaacaaa gattatatga tattgttaag tgcgttaaca |
| 15541 | aattttaaag atgaagagga aattgtgctt catgtgctgc attgtttaca ttccctagcg |
| 15601 | attccttgta agtagcattt aaatgttatt tattttttgt atctgaaaaa ttacaatata |
| 15661 | tctcattctg agtatatttt aacaatattt ttattattta gaaacttgtg gatgctcaac |
| 15721 | ccattcattc atttattcat ttaattaatt tacattcact gacattatac tgaagttggc |
| 15781 | tgtgggcttg gtgctggaga aacaatcgtg gaaaatacag atgtgttcct taccttttca |
| 15841 | gagcttgtag tacaatgggg gacacagata agtacagagg tgattacagt ggcagaagtg |
| 15901 | atggcagatg gcagaagtac ctagagttag gagatcaaat aggaagtgag gcagtgtctc |
| 15961 | ttagcaaaga tttaataagt ggagcttcct gtgcatgaag gtgtgacctg aagtgagaat |
| 16021 | gcaggcaaag tggcccaggc agtgggcatg gttaatgtaa agatgctgga gcaagagaga |
| 16081 | gcagactgcc ttcaagaaga caaaagtagc tcagtaaagg tgtggggtta tgagtgtgcg |
| 16141 | tgcatgcatg cgtgtgtgcc gctgtgcatg cacatcccca aatatcctat ccgtttgtgt |
| 16201 | ttcattgaca gaggcaaggg agagcttgat aagaggcagt aaatgaggcc agagacatgg |
| 16261 | agtggagagc atgaagggcc taaaaagcca catgaaggag tttgaatttt attgtgactc |
| 16321 | ttgattagca ttttaatgag gctttgaaat ttagccacat ttttcaccaa aaatattaat |
| 16381 | cagaagaaat taatttgatg tgtatgctac caatgattgc tattaggcta aaataatggt |
| 16441 | tcatattctg ttttgttttg tattaatggt tcatattctg ttttgttttg taagtgacca |
| 16501 | ttaacacttt gtattttatg tattacttgt gtgggtttct acaggatata catatgcatt |
| 16561 | tatctagtga tattttcatc ctcacacatg tgaagttttg aggattagag ttaaacaatg |
| 16621 | tacctggtat gtaataagtg ttctaaaatc actgacagga ttattagaca atatgtattt |
| 16681 | tatatgtgtg ttgtatacta tatgtaattg catttatggt ttcagatatg gaaatcactg |
| 16741 | tgtcaatctg aaggtgtgag ccttcggtgt aggcagagta aaacccaatg cccttgtgaa |
| 16801 | agaatgcttt tttttggtga tgtttataaa atcacaatgt tttcttatcc acaggaaatt |
| 16861 | aaacactgga aagtgggtgg ggctgaacaa taatagagaa aggccatggt tttacatttc |
| 16921 | tctgagacat cactgccaac aaactgaata tgtttttcat tatacttttt ccttggctat |
| 16981 | atttattcat ttatttattt attttgggct ggaggttttt ggaatccatt gttttccacc |
| 17041 | cacattggac ataactccag taaaaatgtg ttgattcata atgcaaaagt caagaaagta |
| 17101 | gcagctaaaa attaagaaat caaaagtttt taaaacactg attctaactg aaaaacattt |
| 17161 | gcttttcagt ctttaagtct attgttctga gtcaaagcag ttcatttcct tacgttgtta |
| 17221 | attttttttc tatgtttaag cattgtaata tactttttgt gaaaacagtt gattagtttt |
| 17281 | ggttgtgcca aaacaaatac taaaatgttt tgcaaacagc ctttttttaa acaaaaaaag |
| 17341 | aacagttaac atttgatgca gagatataca tgttttctcc atgtaggttc acacctcact |
| 17401 | tcctttattg attaattgct ttttctggta gagtctttct ttcctttctg ttttacctgt |
| 17461 | gtttgtccct aagacttata ttttaatatt atgcctcctc tctttcgttc tcccatcttt |
| 17521 | tcttccacct attttggagc cttcaggaag cttgattttg ctgccttgta cattggttgc |
| 17581 | ccttctggaa tggaggaaac aggtcatagc tgattttaac tgttccatct ggtgacatat |
| 17641 | tcttgatttt ctttcttttg gttggggaaa aaaaaacaat gcaaaagtca ttctccaatg |
| 17701 | gggttgagcc tcgttaagaa atagaccctc cacaatggtt gaactagttt acagtcccac |
| 17761 | caacagtgta aaagtgttcc tatttctcca catcaaaaaa aaaaaaaggt aagcaatata |
| 17821 | acatgagcca tatctaatag gacttcagaa attatctatc ctatagttcc aggatgacga |
| 17881 | tgatgatggt tgtgataatg atgaagattg tgatgatagt tatatgggag ataaaacttt |
| 17941 | aagcacttta catattaaat tctataatat tcaccacatc tattaaaata tgttacatta |
| 18001 | ttgtccctat tttaccaaca agaaaactga ccaacaagat taaacaacgt gactaagttc |
| 18061 | acacaacctg taacagcaga atctatgtca atcacaacac aattagcagc tatttctgtg |
| 18121 | gcaattttca ataaagatgt gtctggaaaa aaaaaaaaga aatagaccct ccaatttatt |
| 18181 | tatctgaaaa cttatgacca atacattaca tttccagact ttcattttca gtacttttcc |
| 18241 | tttcattttc agtactaaaa gtactctgaa tttttccttt ttttgatctt aaggctttaa |
| 18301 | gccaagaaac aggaataaag taaattttcc ttaatgccaa agattagtcc tacaccccat |
| 18361 | tatgttatta atgaacagca tagtattttt tacagctact taaagaacat gatgtttaaa |
| 18421 | tttggaaatg cagtcattat gctgccatct atttacagtc tatataagac gtctttgtat |
| 18481 | gcatatttga aaggagaaca tggttacctt attgataatt atgatctctt taaattcagg |
| 18541 | caataatgtg gaagtcctca tgagtggcaa tgtcaggtgt tataatattg tggtggaagc |
| 18601 | tatgaaagca ttccctatga gtgaaagaat tcaagaagtg agttgctgtt tgctccatag |
| 18661 | gcttacatta ggtgagtttc ttagttaata tgtcatcaca cactgtatga tatacatata |
| 18721 | catataaaac atatatatgt tgcataataa tggataagta gcatattgac atactttgaa |
| 18781 | tgaaaatatt gtaaaatccc agaaaaaata aattaaaaca aaaagaaaat actgtaaatt |
| 18841 | accaaactgt tctgctgtgc ttagatggac ttttaaaagg agtgtcaaaa atagatgtgt |
| 18901 | agaatgtaaa agaagtatta tcttaatctt atttttatag atgtagttct atagatgagt |
| 18961 | ttttttattg ttgaggctat atttaaaata taattatgta agaattgata catacaaaaa |
| 19021 | tatgcataac atatacaata taaagcataa tgcaaataac tcactatcca acttaaatgt |
| 19081 | tgtatattcc cagtggggga agctaccctg ggcttcttcc tgcccttgct ttctctatag |
| 19141 | aggttaacac tatccagaat tttgtgttta caaatctttt gtttataaat atgatttact |
| 19201 | acattttcat gtttctctaa gcaaaattgt taatgtttgc ctgcttttgc ttcatcaaaa |
| 19261 | tgtaatcata ctgtatgttg tcttctgcaa ttttcaaata tcagtgctat gattataaga |
| 19321 | atcagaaata tttttgcatg tggttgcatg tagtattcta ttatttggaa ataccacaat |
| 19381 | ttatttttcc atttttctat ccatggacat ttggattctt tcttatttta tgctactact |
| 19441 | atctgtgtta aaacagataa ctgaaaaaga acagttaaca tttggtgcag agatatacat |
| 19501 | attttctcca tatataaata agggttaata ttttaaaaaa tatttatttg ccattggtat |
| 19561 | gtccttcttt gtgaaatatg caatctgtca tttatcaata ttgttcacag gatagtctgt |
| 19621 | ctttttaaaa ccgattcata gattctggat aataatgttt tggtggtagg ttatacgtat |
| 19681 | tgaaaatacc ttcccttgaa catcacactc tggggactgt tgtggggtgg ggggaggggg |
| 19741 | gagggatagc attgggagat atacctaatg ctagatgacg agttagtggg tgcagcgcac |
| 19801 | caacatggca catgtataca tatgtaacta acctgaacat tgtacacatg taccctaaaa |
| 19861 | cttaaagtat aataataatt ttaaaaaaaa ggagatgaag aggtagctgc aggttgactg |
| 19921 | agcaagggtc attgtctatt tgaagtttca aaggtatctc tgaaaataaa cacagttttt |
| 19981 | tgcaagagtg aaaaaaaaaa aaaaaagaaa ataccttccc ttgacttgta gcatgtcttt |
| 20041 | tcaccgtctt tatggtggct tttgtgatat agttaaattt aataatcagt tcctttgtga |
| 20101 | tttactcttt tttatgtatc ttgtttaaga aatcttaatc tgtcctttcc ctaagataat |
| 20161 | aaatatattg tatattttat tccaaatctt actcgtttgt taaactgttg cagtttgttt |
| 20221 | ttgtaaagaa cccaattccc cctctttttt tcagtgtgga gagctagtta ccatcacagc |
| 20281 | acaatttatt agaggttcac ctgtttccca gtcatgtggt atataaatat gtaaacatat |
| 20341 | atgcttatgt ttctgtcctt ggcgttctgt tgcattaatc tgatttgtcc gaatcagatt |
| 20401 | taaatctgat gttaaatata acactattta aattaacagt tctgtaagtc ctgatatctg |
| 20461 | aaaagctaat aggtcaagtc accatacatt cttttttagg cacagcttgc cttttttctt |
| 20521 | gggcctttac tattccatgc aaattacagg attaacttgt ccagttcctt ggaaaacact |
| 20581 | gttgagattt tgactggaat tgcacaaaat atgtggatca ttttaagaag aactgacatc |
| 20641 | tttacattat aatttcttct atacatgtct tgcacatctt agtttttgct acgtatctta |
| 20701 | ttttttatta ttattgtaaa tggtatttct ttttaaatca tatttttgaa ctgtgttttc |
| 20761 | ataaagaaat gcaaatgatt ttttgtatat tgaactaacc tacctttcta aactcttcgt |
| 20821 | taattctgac aatttgtttc taaatgcact taagttctct acctagcaat tatataattt |
| 20881 | gtaaataatg acagtctgag ttcttccttt ctgcttctta tattctttat ttcatttcct |
| 20941 | tgtcttctta catgagtcag aattattaat atagtgttaa atagagccat tgatactaga |
| 21001 | catccttgtc ttgctctttt tcctgatttt aaacagaata tttttaatat tctcccatcc |
| 21061 | aaaataatgc atcttacaag ggtttaaact tttaaaaaaa attttgttaa gaaattttat |
| 21121 | tttatttctt ctttgctttt gttttttcaa tgtgggcttt taaatgtttg cttttatttt |
| 21181 | tacaatgtgg gcttaaaaat attaaaatat ttaattttat caaatatact taaaatgtag |
| 21241 | taagtctttt ttttcctttt cattatgtta atatgaagaa atacatctac aggttttcta |
| 21301 | atactaagct actgtgcact tcctgataaa tttaacttgg tcatttatta gatttttaaa |
| 21361 | aacacttcta aaaaatcttc tctgtttatt ttcacatata ccaggtgagt tttggcagcc |
| 21421 | tcttgttttt tcttttttac cctttctttt ttgttaaaaa catcctttta tttatttaat |
| 21481 | gattaataca tggctatttt gtgttttgta tctgataact atatataaag ttctggggag |
| 21541 | tctaaaccaa ctgcttctag gctgactgcc tgtcagttgc ggagccttgt tttcttgtat |
| 21601 | ggttgtgagt ttctctttta ttgagcatcc tgaggactta aattaaagat gctctttcag |
| 21661 | aggtgtttgc caggagtcag agcacaggac tgacctggga gcagtttagg atatgatcca |
| 21721 | ggcttaatat gggagactct ggttgagacc ttaccttgca gagagtctga aactggtttg |
| 21781 | ttgaatgcag cgccaggatt catgctttcc cacaagacta ctctggcgtt caactcacag |
| 21841 | ctcttgtttc agcttctttt tgaactccct ctgcccctca acacacacct tggaaatttc |
| 21901 | cttgatattt ttgtgaggac aacaatgcat ttaaaagtga gagtggttgc tgaataataa |
| 21961 | ggaatgatcg ttaactgttg gacattgatt ctgatgacat tttctcaaaa ggataaccag |
| 22021 | gaatattttg tacaaaatta ggattattat aataggcatt tagtttttca ttgttacaaa |
| 22081 | ttttgaggaa aatcattaat catttgaaaa aactaattga catgtctcca ttgtagcaac |
| 22141 | ttgtattttc acttctagta tcatgattat atcccctgtg taatttggaa attgatttta |
| 22201 | gcattaggaa atttcctagt ttcagttaaa atgaattttt tgtaagctga attctatttt |
| 22261 | acatgcacaa ctttagtttg ttatttcttt ccttgacaag catttattga atgttgttat |
| 22321 | atgactaaaa ctgtaagatg ataatgtttt aatattttca catttctttc atagcttcca |
| 22381 | aagtgtatat atatactcac atacttcata tatatgaatc tacttatata ctgtatataa |
| 22441 | aaatatgtat ataaatatat acacattgta tataaatgtg tatatatatt tacacatgta |
| 22501 | tataaatata catacaaatg tatataaata tacatattta tacacagatg tatataaata |
| 22561 | tacatattta tacacagatg tatataaata tacatattta tacacagatg tatataaata |
| 22621 | tacatattta tacacagatg tatataaata tacaaatata aatatataca tttatatata |
| 22681 | aatatacata tttatacaca gatgtatata aatatacaaa tataaatata tacatttata |
| 22741 | tataaatata catatttata cacagatgta tataaatata caaatataaa tatatacatt |
| 22801 | tatatataaa tatatatatt ttttgatacg tacaactatc ttgggagatg ggatattgtt |
| 22861 | attatttcca catttacaga tgaggtcctg tggttcatca atctttgtgt tttattcaag |
| 22921 | attatgtaag cagtaaggga tgaaatcagg ctgagaaccc aaatttcttc atccctaaac |
| 22981 | aaagtatttc ttctaaacat ggtatccatt tactagttta tctctatcag gtgacccttt |
| 23041 | attcattatt tttcatgaga aggttgtagt tgtacaaagt ggctgatatc tgataatgtt |
| 23101 | ttaatctaat tcaaagtcga tttcttaaat ccaggtgtca gagtagcaca ctactgtaca |
| 23161 | attctctgtt tcatgtttta caatttacca cagtcaagtt acaacttgca catgttacat |
| 23221 | taaaatgtga attcacctta atttcttgaa atgagccaga agaagaagtg gttttgtttt |
| 23281 | gtgatcaggg aaatgctact tgactgccaa attgtctaga acagcacatt aaagttgctt |
| 23341 | gattttatac tgttaaaatt aaataaaaca tggcaactgt catgtcatat gtaacttttg |
| 23401 | tattattctg taactttttt tgaaaataaa aagtgatcaa attgatcttc aggtaaagaa |
| 23461 | tcttttttct cttgattatc tcttagtgga tgatgatttg ttccatttaa tgggtagaga |
| 23521 | atttattgtt gctgttattg tttcaaaagt agctgaatga aactcttaac tttttcttca |
| 23581 | tagttaaaat taaaacctca agtaaataaa tatttaacgt tttgccaaac tatgtaatct |
| 23641 | aattatgggc ttaatgcatt taaaggcttt gtataatttg ctacgtattt tcacacaagt |
| 23701 | tacctgaaca taagtccagt cttcctgctg ttttctgagt cacacagtga ttcagtgacc |
| 23761 | gaacagctgt tggcagtggt gtgggtataa tagggaaaag agactgatgg ggacaaccca |
| 23821 | agtttagaca agctggtaaa agtagaagaa aatcttcttg aaaacactag tgatcactaa |
| 23881 | gggctgtgga gaatttttgc ttggtgggtg aatgtggaag aggcaacagc atgggaaggt |
| 23941 | gttggtaaag gagctccata cttgcttaaa ctgccttttg attgtgaggc cgttgatgaa |
| 24001 | tatttagttt gggctttagg ttttttatga tacaggatat tttccatttc tggctttgta |
| 24061 | tctcagagat cacttagtta cacttataga tgaataggag tttcaattcc ttgttttaga |
| 24121 | aagaagcttg gtaactgtta gtgagttaca aataagccaa atagaagaag gtacatattt |
| 24181 | ctgcagtatc aggtaaagtt tttcctcata aggatttaga ctcttggata tcatattaat |
| 24241 | tctcagaaga gtgggtataa aaaggtatgg gacttcttcc tggggtgggt tggaggtggt |
| 24301 | gaaatacctt tttttttttt tttctgagat catcatagac aagatcaaat aatggtaaac |
| 24361 | atgccaatga attttctaag cactattcct ttaagtgaaa gaagagtgtt tcagtaaaat |
| 24421 | gatttaatat tgggtcttcc aaaagatgga tttaagagtt tcaactttaa aagacagaaa |
| 24481 | aattaagtta ttttacacaa tgaatattgt cgtgccgtgt gtcacagaca tgacatgaga |
| 24541 | gggaatcaga gaacatacag ttaatacaac gcaaactagt atcattactt ttgctcaatc |
| 24601 | acttccattg tctaagtaag ataattaaag gacagcataa aataaaattt caaaacttta |
| 24661 | ctcaatcata ttaagctatt ttaattaaag taaatgtttt aatgccattg aatattcatc |
| 24721 | accattcaaa attattgatg taaatagtgt tatatgttaa aggtaattta acttccatgg |
| 24781 | atgagaattc agctaatgtt tcacttaact tttaggtaat tttttcaata tcctggtatt |
| 24841 | aaacgaagtc catgagtttg tggtgaaagc tgtgcagcag tacccagaga atgcagcatt |
| 24901 | gcagatctca gcgctcagct gtttggccct cctcagtaag taacttcact aaaaagggga |
| 24961 | ttcttacaga ggcatttgac atcaaatatg aacattgtaa caagagaatc atatgtacag |
| 25021 | atggaagcat tcaatgcctt ttctgtcctg tgtagctcat tttccagtag aggatacttt |
| 25081 | caaggaaact aacagttgtg acaaatatac acatctcaat gtagagtttt gctttacatc |
| 25141 | attcttgatt tagctttgtc attaagcagc taatctgttt taaaaaaatt tttatttgtg |
| 25201 | cctgggcatg gtggctcacg cctgtaatct cagaactttg ggaggccgag gcgggtggat |
| 25261 | cacaaggtca ggagttcgag accagcctgg ccaacatggt gaaaccccat ctctactaaa |
| 25321 | aatataaaaa ttagtctggc atggtggcgg gcacctataa tcccagctgc tcgggaggcc |
| 25381 | gaggcaggag tatcgcttgg aactggaggg taggagttga agtgagctga gattgtgcca |
| 25441 | ctgcactcca gcctgggcaa caagaatgaa actccatctc aaaaaaaata atttatttgt |
| 25501 | gttttaagtt ctggtgtaca cgtgcaggat gtgcaggttt ttcacatagg tagacgtgtg |
| 25561 | ccatggtggt ttgctatacc tattaaccca tcacctaggt attaagccca gcatgcatta |
| 25621 | gctatttttc ctaatgctct cccttctcca gtcccatccc ctaacaggcc ccagtgtgta |
| 25681 | ttgtccctct ccctgtgttc atgtgttctc attgttcagc tcccatttat aagtgagaac |
| 25741 | acgcagtgtg tggttttctc ttcatgtgtt agttatctga ggataatggt ttccagctcc |
| 25801 | atccgtgtcc ctgcaaagga catgagataa ttccttttta tggctgcata gtattccatg |
| 25861 | gtatatatgt accacatttt ctttatctag tctatcattg atgggcattt gggttgattc |
| 25921 | catgtctttg ctcttgtgaa tagtgctgtg atgaactaat cttttcaaat aatcctcctc |
| 25981 | tcgtctatta ggtttttttt ttttttggta ccttcttcct cattttatta tttatctgga |
| 26041 | taggatggta gcattatgag acgtataata tattaaaaat tatctttata attgaccaag |
| 26101 | gcttctccta aagcacacct cattctgttg gtaatatttc aaaatatgga cttagagttg |
| 26161 | gtcaaactgt taagtagata atatatataa tgtttttata tatttttcaa tttttttcaa |
| 26221 | gctgagacta ttttcttaaa tcaagattta gaggaaaaga atgagaatca agagaatgat |
| 26281 | gatgaggggg aagaagataa attgttttgg ctggaagcct gttacaaagc attaacgtgg |
| 26341 | catagaaaga acaagcacgt gcaggtagga ctctcataaa tattagagtt attcaaaatt |
| 26401 | atgttttcca gtcatttata ttttgacaga tttctttttt ctcccctaat ccaggaggcc |
| 26461 | gcatgctggg cactaaataa tctccttatg taccaaaaca gtttacatga gaagattgga |
| 26521 | gatgaagatg gccagttagt agttttgatt ttatatgata gaaaatttca gttatatttt |
| 26581 | aaatcaatac ctataaaata ccttaaccgt aacttttatt gttagaaata tttttgatat |
| 26641 | aggcatttag ttttagatgt tgctgcaaaa tagtagtagg tatgtagtat tttgatctca |
| 26701 | tcaccttcag gagttagaaa aggtagaatg agagttatta ttgagagatt tggaatcaag |
| 26761 | ggtcatttgg taattcatga atcatggaga aagaatcttt ctattttctg gctgatcgtt |
| 26821 | ttaaaatgcc atattaattc atcttgggtg atagaaattg cagagccatc tgtgatcttt |
| 26881 | tcttctatgg tgactagcca gcaggttgtc aatatcagaa attagatttg gttagagagc |
| 26941 | ttcctatgat gagttcccaa ctgatgtgac agagttgacc tgtcttcttt cgagagggtt |
| 27001 | atttgaagct gtcatctctg gataactctt tcaataggag tgccattcaa acatcataag |
| 27061 | accggcactc tctcccaaag atacaagctg tagcaaggag ttttgtgcat atcaggtttg |
| 27121 | tttcatatcc gtgagccttt gtgttttatg gcaactgatt gattatactt gtgctatttg |
| 27181 | caatgggtat cctctgggtt ttaaatagtg aatgacttat tttggaaaca aatagtagta |
| 27241 | ttctatgtct gaaatcttga ccgtctattt gtttaattat ctattgctga taagaaggga |
| 27301 | attaataaca cagatgctac ttaattaaat attttcattt tgacaagaaa cagtaattct |
| 27361 | tttgaaaact atgctaaatt ggcatcttaa taatctcatg ttgagcaagg cttttggaga |
| 27421 | ttagggtaag ggagattcat gtcgcagttt ataaatttca gttcatagga tacctatttt |
| 27481 | tcaatatcca taagataact ttaaaataaa tatattatta aaacaaagaa aatatattta |
| 27541 | cgttatacat ctttaaaacc taccttgctt ctttacaatt ttcagttttt cctctcttgt |
| 27601 | ttccattctc ttctcctcac tttacctttt tcctcagcat ctctttacat atctgctgag |
| 27661 | cttttttatt tctctctgct gcatatgctt ttgaaaaagc ataaaaatac taatttgtta |
| 27721 | ggatttaatt aattcgagtc ttaaaaaatg aactataatt cactcttgta agtggaggtg |
| 27781 | gcatgaaata ttgtttatat gctcttaatt gttgttagag atatttgata atggcaagtg |
| 27841 | agaatttgag atagttattt aaaagattac tactaacatt ttgtttgaat ttttgaaagt |
| 27901 | ttcccagctc atagggaagt gatgctctcc atgctgatgc attcttcatc aaaggaagtt |
| 27961 | ttccaggcat ctgcgaatgc attgtcaact ctcttagaac aaaatggtaa gcagtgggcc |
| 28021 | atgttttcaa ataaagggaa acacattttt gtggtatttt taattataga agctatatac |
| 28081 | tgtgaaaaat ttacataatt tataaagcta tatattgtga aagatatctc tatgtgtaga |
| 28141 | gatgtattga catatggatt atgaatatat aggtaaaagg atgaagaata aaataaacat |
| 28201 | ttgttgtata tttttccgga cttctatgtg taaactcaca catgacatat acaaaatttt |
| 28261 | atgtattttg tagaaatggg atcatattat actcttttat aaccaaattt tatttattct |
| 28321 | cttttttatg ttgatacatg tgtattctca cattatcatt ttatttttaa ttttttaaat |
| 28381 | taatttttgt ttttagatat ggtctcactc tgtcacctag gctggagtgc agtaccatga |
| 28441 | tcatggctca ctacaacctc aaacttttgg actcaggtga tgctcccacc tcagcttcct |
| 28501 | gagtatctgg gactacaggc atgcactgcc atacctggct aattttttgc agagatggtg |
| 28561 | ttttgccata ttgcccaggc tggtctcgaa ttcctgggct caagcaatcc atctgccttg |
| 28621 | gcctcccaag tgctgggatt acaggcatga accactgtgc ccggcccaaa ttataatttt |
| 28681 | taatagcttt gtagtttttc agtgcttgcc tgtattctgt tttatgaacc aatttcttac |
| 28741 | tgatgaagtt ttagtttgtg tccagtggga tgtttctgga acccatagta aacaccagtg |
| 28801 | atcactttcc tcctctgttt tcctttatac atggttccta ttattttctt gggaaaattt |
| 28861 | catagaaatg caactgggta gagagctatt caccatttta aaatctggta aaattgtcta |
| 28921 | ttataaattt ccacactata cttttttaaa aaatcgttct ttaatgtaat tcttataaat |
| 28981 | cttatagctt tgtataatta tgaagagaaa aatggcttgt atccctttag aaagacatga |
| 29041 | gttttaagat tatggttcag gctgctcaaa tttcttcccc cataaacagg aatactgcca |
| 29101 | gaaatcctta ggtgaaaact catcataaag gcattgggac ttggcagctt ttgcaggaca |
| 29161 | tttttagagg gcaaadaata gagaaaaaca ctgaaagtca gagacagaga ccagtgtaag |
| 29221 | catcagcttt taatagagaa actgggtagg gtggaaaaaa aataaagcaa ccacctcatg |
| 29281 | catgtttctt tatattatta ttgaagtcaa ataaagagga aaatcatttc ttcttcctct |
| 29341 | tcctcctttt tcatctcacc tctccaatgg cactttaata aaacgcttgg agtggccagg |
| 29401 | gcactgacag acagacaggg gctgctctca aggataatga gtcaaagggg aaggagaggg |
| 29461 | aatcgctgtt ctcgaatctc tcttattcta ctgtgcagtt aaagaggtct ggacagggat |
| 29521 | ttcactcctg aaaatgagga ctggactttg tggcttctgt tggggcacct ttagagtgga |
| 29581 | ggtagacttt tactatgtac agacaacatt gtgttggtga catcattcat aaccacctgg |
| 29641 | aaatctcctt tgatatgcaa atcaaacaac cataactttg tgaaatttcg actgcttcct |
| 29701 | attgtggtgt ctgaggactg gttacattca gagtccaccc tgatgtcttt gttcagtttt |
| 29761 | ctgctctttc tagttcctta cttctttgtt cctgatcacc tgtcaagtaa aatgtcctca |
| 29821 | gatccttttg tattgtcttt ggagttctgc cttaataaag catgaagaac ttgagtagct |
| 29881 | cgttcatcaa ctttcttggg caatttctca ttgaaagaca cttgggtgtc tttgggtgtg |
| 29941 | cagagctgag catggcttta tgtttttaga aaaaatggct acattggcag gcagaagaac |
| 30001 | tgcgtccttg gaatcatgga ggtcccaagg ttgcatacat tttgtgtgac atttttccta |
| 30061 | ttcaattaat taactaatat ttattgagct cccaagtgtg tagtgtctgt aggcacttgg |
| 30121 | gatgcattca ttaagtaaaa atcccaggct catggagttt aaactgtagt agggaagata |
| 30181 | ataagattaa ctaaaatatg taatatttga ggcagttaag aataaaaaat gaagcaagga |
| 30241 | aggagaatat gatatgttag acatcagagg agatgaagtt gtgaataggc agccaggaaa |
| 30301 | gaaggtgact attgagtaag acctgcaggg cgtcgcatat tgttttttgc ctctgaaagc |
| 30361 | agttaatttc ctgttaaaat ggagtggatg agatcaagag tattacgtag atagctggta |
| 30421 | aatgcgatag tgtgtaaaat gttctataaa gtctaacgtg actttatgat gaaatttctt |
| 30481 | cttctaggtt tattgcttgc aattttcaaa ccacacattg ggttactgtc taggatagtg |
| 30541 | attcttaaag tgtggttcct ggaccagcag catttgctgg gcgaacttga taaacagtgt |
| 30601 | aaattcaagg accccataca gaccttctca atcacaaacc ctggagttga gacccagcaa |
| 30661 | tccatgtttt aacaagctct ccaggtgatt ctgatgcaca ctaaagtttg agaaccacta |
| 30721 | acccagtgtc atttttgtct tttaaagtgt cttcttggct agaagctagc cactttggga |
| 30781 | aaggttatta caacttctga tgtgatcaag caaagtaacc aactctttat tgtatcttaa |
| 30841 | tatgtgatat tctgaatgtg tttaaaaggt atgagttttt caggctctgg cagtatttta |
| 30901 | gaatgtgtat gtgatttcta tttatttcca tgttttgtcc tatcttctta agatagacta |
| 30961 | cttattttaa aagcagtact taagttaaaa ctttttatgt ttctttttct gccactttca |
| 31021 | aagtgttgaa tcacagtgtg taatgttgga actgatattt ttatagcggc ttcaagacaa |
| 31081 | ttgatattta tgtggaaact tgaagacagt aggtttatgt ttagtgaagg aagtttatta |
| 31141 | caaagaggaa aattggccag ttgtggtggc tcacgcctgt aatcccagca ctttgggagg |
| 31201 | ccaaggcagg aggattgctt gagctcagga gttcaagacc aggctgggca acatagtgag |
| 31261 | atcccctctc tacaaaatat taacaaaaat tagccaggca tggtggcaca cttgtagtcc |
| 31321 | ctggtacttg gaggctgaga caggaggatc acttgaggcc aggaggttaa gactgcagtg |
| 31381 | agctatgatc atgctactgc actccagcct tggcaacaga gagagatgct gtcacaaaaa |
| 31441 | gaaacaccaa caaaaaaaga ggaaaattat tccttaatca ttattgctgg aatatagtta |
| 31501 | ctttccacaa atagtgaagt gccagttgta aagcatatct atatgtttcc tagactttgg |
| 31561 | cattactttg tgaaaataac tgtaattact tatgttctat gtaaatgctt tccattcatt |
| 31621 | tgtatatgat ggcatatata gaaattataa tgtttgtaaa gtccactggg ataaatggac |
| 31681 | aaagcagctg aaggctgaaa gcaaccaagc cttttacagc cccttcattc cccacactcc |
| 31741 | caaaaagctg agtgaatggt cgatacctcc acatgcttat aactcattcc cagcccacca |
| 31801 | gtgtctagca tatctggtta gtcttagctt tatataagtg cagttatttg tgaacttgtt |
| 31861 | ttaagtattg gaatacaatt taactttcat tcttattttg gagaccatta tttaaacaga |
| 31921 | tttctttttt cctgcaaaaa cactcttttc acaatggaca gagacacggt gattacatta |
| 31981 | aaaccatcta ctctatgaat aaaaatgtta aaaccaaaat cccaacaaag ggttaataaa |
| 32041 | ggcaaaaaaa attggaaatg acatgtgttt taaagaaata aacatgaatt atctttaagc |
| 32101 | tgtcaatgaa ctataaatta tgtgtgctct tgtatatgct ttcctgtaaa tttggactat |
| 32161 | attaatattc taaagcttat ggtaaaatta tgaaaatatg ctttcatatc tataagtaac |
| 32221 | attttaaaaa atctcagtta atttcagaaa aatactgtta tcaaaaggaa tacacctgaa |
| 32281 | tgttttggag ttaatgcaga agcatataca ttctcctgaa gtggctgaaa gtggctgtaa |
| 32341 | aatgctaaat catctttttg aaggaaggta atatagattc attaacttgt acagaatata |
| 32401 | tcatattggg ccaggtagaa tatcaatatt tcaagcatat ttctaacaat gaaaagaaaa |
| 32461 | agaaaaacat aagacacttg aaaactgaag cattttgcaa tgtaatctcg tgtcactagt |
| 32521 | accatagact tactttatct gaacactgaa aggaatggca agattgtgga aacatgttga |
| 32581 | aggtttgctt ttgaacctga tgcttgatgt tgactatatt ttgaaaagtg gtaattgtat |
| 32641 | agcacatagc atacagcagt ttttctaatt attgtgtgtg tgaaagttat aaaagataaa |
| 32701 | atcagtttat ggctaaattt tgctctttca caacgaatat attattcctt catctgaatg |
| 32761 | aactttgtct tctcttcctg ctctcaatcc ttagttaggg aaaattttaa ctacatctag |
| 32821 | tccaagtgca gagatgctaa gattatatag ctggcggtta gtggcacaac agagaccata |
| 32881 | tcctttgatc tatgtgtgga tggtggtggg gcagggtggg atgaggtggg ggtgaggggt |
| 32941 | ggtaggtgtg ggcagaactc tcatgtgtaa aaaaaataat tggcacagaa gttgcagtga |
| 33001 | aaactaattt tgttcctggt tttgccacta atttgagcca actgtttcat ctctaaaact |
| 33061 | tcacgttcct cattgataaa gaggaatgat aataacaact ttgaaagttg taagcttaga |
| 33121 | atgtaaggat taaataaatt aatttttaca aagggattag gataatgcct gctgcatttt |
| 33181 | aagcactcaa caaattgtgt ctattgttgt tatactgtta ctaagtgtga ataaatgaag |
| 33241 | tgcatatagc atgaaatgta gcgtaactgc agacttgtaa gaagtagggt tacactgttt |
| 33301 | ttaacatcag tctaactaat ctatgtttat atatctttct aagctgtatt tctcttattt |
| 33361 | aagtgttgtt tttgaacact gagatgaaaa gtttatctta aatgttgatt ttaatggggg |
| 33421 | cggaagtgtg caaaccttta caaatgaggc aaaaacacag cggaataaac tccagtctag |
| 33481 | gattctgtag attctgggca aggcatttaa tgtttctcgc tgcatgctct tacgtaaaat |
| 33541 | gtgtacagtt gcagccctgg agattccgca ttggctctga cagtgtgtct gcccctacag |
| 33601 | aactcatgtg agtcgagaga ctgataagta aacagattat tataatacag tctcagaatg |
| 33661 | cagtggcagt agtgtgtaaa agacgcaatg gtaagagtag agtggactca gctggggtta |
| 33721 | cccaaggagg ggaggctcca atggagggat gtgtttaaac tgggacttta agttggaaaa |
| 33781 | gaaggaatgt atctcagtgt ccacagaacc atgcaaagtg agaacatggt tctgatatgc |
| 33841 | acaagtttca gttaacaagc aaagcaagga ttgactgtat taaagttcat agtacctact |
| 33901 | gcattctagt caagtgacat ttgctcatat gtaaaagaaa gaatagctta aatacctgag |
| 33961 | agaaaccaag actgtaaaac aaattaaaaa taattaaaaa taccttataa gaagtccagt |
| 34021 | gatgttaatc tggaagagga aggttcgtgt gatgtaaaga ggccttgtct tggggtcaga |
| 34081 | caagtttggg taccagcctt gtctctgtca ctttctagtg ataagacctg aatatttaac |
| 34141 | ctctatctgt ctaagttctt catgtagaaa atggggataa taacacctac ctgctgggat |
| 34201 | tgttgttatt gacccatcgt aggtcagaag gatgttgtta gttttatgaa gtgaaataat |
| 34261 | tcccggatta ctatgaattc tatcttatga gttcaaagtt tagacaatta aaattatgta |
| 34321 | tgctcatact actgatttca aatgcatttt catatagtct ttcctgataa aatatattgg |
| 34381 | ttctgccctc ctgtacttat ttcaatttgg tgtttatacc attgaatcag atcagtcttt |
| 34441 | caataagcat gccaatttta tatccccagc aacacttccc tggatataat ggcagcagtg |
| 34501 | gtccccaaaa tactaacagt tatgaaacgt catgagacat cattaccagt gcagctggag |
| 34561 | gcgcttcgag ctattttaca ttttatagtg cctggtaagt tacatagttg attgtgggaa |
| 34621 | gagataacaa tttaaatgga tttttgattt ttcatgaaat agcaatattc taggcaaata |
| 34681 | ttaaaagact agtttctgtc gactaaatgt aaatctttct gttaaaccaa aaagaggtta |
| 34741 | aatatgatgc agaagagtca cttagattaa tttttataag aaagcaatat gaattcagta |
| 34801 | atttatttat acaaagtaac tacaatgtaa aatgtggagc ttttattttt aaggagggtg |
| 34861 | ttcatctctg ataattcttt tctatttttg ttgccatgac ctgagttcaa gctttttttc |
| 34921 | tcttgtttga attgtactat tagctaattt tcatacctgt tctcttcctt catttctatc |
| 34981 | cttttcttac tctatgattg aattaatctt tctccaatgt ggcttgtact catttacctc |
| 35041 | aatggcttcc accctccacc cacctttaat tgttactgac atctgttatc accttatttg |
| 35101 | ttctccaaag cccctttaaa acacctatgt ctgtattcat ccaacacatt tattaagtgc |
| 35161 | ttcttatgtt ctaagcactg tgatgctgtt aaactttaaa agatgaattg gaaaaaaagc |
| 35221 | acaactgtac tattatagga gcttagaggt agatggaagt gtaaatagat aattaacagg |
| 35281 | cagcgtgaga gaggcgatga tagatgcata tataaaaggc tttgagaata tcgatggagg |
| 35341 | cacaaataat ttcttcaaag gagtaggaca gagattgtaa tatttgaggt ggaagggtag |
| 35401 | atagagctgg cctggtagaa aggccaagga agacatttca agcagaagaa agtgcacatg |
| 35461 | gtcgggggtg agtgaggcat ggaggataag gagaggagtg cttggggatg agtgtggaag |
| 35521 | gaaaggctga ggcccaaaga aaagggcctt gaatgcagtg ctaaaaattt tttgcctttt |
| 35581 | aaaaatggaa gccaacccaa gtttattaaa attgtttgca gattgtagtg tcacaccgaa |
| 35641 | atttgcaact aaagacaata gcattgtggt gcagaggatg atagacaggg agagagacta |
| 35701 | gatacaagga gattgggttg gaggtccatg gtagtcaagt gagacccagt gaagggaccc |
| 35761 | agcaatggaa acacagatga gagggcagat tggacagatg gttgggagat ctgtttgatg |
| 35821 | tgacatcgtg atccctaagt atgagggatg atactttgtt ctgtgaagtt cccaatcttt |
| 35881 | tgaagtcatt tttgctttgt tcttttaaaa tgaaatcttc ctaaatctat cattttctct |
| 35941 | agttatatag gtattttgtt tctctgtata agagtattac tcaatataaa agttttttca |
| 36001 | aggacagagc tttcttcagt catttttttc tgggcccccc agtcctctgg tggctattca |
| 36061 | atcagtattt taaaaattga atcatggtgt cctattcata gtttcagctt agttttgaga |
| 36121 | cgtaatgtaa cagatgtaat tttacttgaa aatattattg catgttttat tgaattttat |
| 36181 | ttttagattg taattaaaaa caataaaaat gccttttgat tatccttaaa gttgacagcc |
| 36241 | ttattctttt gagggaggtt ttgggtttta aagataacca aaggacactc aaaaaccgtt |
| 36301 | tctgttagtt aataaaataa tactctttta gtccaaaagc aagttttgaa tacagtattt |
| 36361 | cttttctttt attgttctta aactgatcct gaaggaaaat tgttagttaa caatcaatca |
| 36421 | gatgtattat gggtgccctt gaaaataatc acttgaggac tgtacttaat gtaaaaaaat |
| 36481 | acattttata agcatatcag tatgtaagta cattcttcta gtaggtaaag gcttaatcat |
| 36541 | ttactgtatg ctaacatatt atactaccac agtacaccat actgtactgt acccaacgta |
| 36601 | atgtaccatg ccgtgcagta ccgtatcatt ctaggtatca acaagtaact atggtagcaa |
| 36661 | tactccagta gttttgaggt tgaatatgat tctggtttga acatgttaag ttgaggttct |
| 36721 | tatgagatac ctaggtgtac atgtattcat ttattagcta gattatattc aggtttttta |
| 36781 | gtggcaaaga tgtacattat ggcaaatgtg tataatttag ggtccaatta aggatagcat |
| 36841 | attgcatcta gtgcatatat cttaagtttc tttcaatcta ttactgtact ttttctcttc |
| 36901 | tactttataa taacttgtta tatagacatt gagctggttg ctttaagaaa ataataatga |
| 36961 | agacccagtt actttaataa aatatcattt gacttgtttc ttgagttact gctagtttta |
| 37021 | gaggaaataa tgtgaaattc tttaagaagg gatataggca aattgggaag tatatagagg |
| 37081 | agagtgatta gggagataaa agcacatcaa aaagagcaat aagaggaact gagattgttt |
| 37141 | tatttgaaga agagatgact caaagggata tgaaactcta ttcaaatcta cttacttata |
| 37201 | accagtaaca gtaataatac caaacaatat ggaggactta aaaagtatta ggcattgttt |
| 37261 | gaagtatttt aagtatatta tctcttctga ttctaacagc actccatgag gcagatatta |
| 37321 | ttattattat tattttggta ttgtatggac tacaaaactg aggcatagaa ttgtagagat |
| 37381 | ttttaaggtc aggcctgata taacagcacc agattttcag cttatgcagg ctgactccag |
| 37441 | agtaggcatt ttaaaataaa tatttgtgca acaatcttgt cctgaacaac tgttgtattt |
| 37501 | aaagcactat gtctagaatc cttaaggtat cgggagatga tgagattatg gatactgctc |
| 37561 | tcaaaaaaat ttacagtcca cacgacctaa aggactgtta tgtagaaagg aaattatatc |
| 37621 | catttgaata gtaggacctc aagtgataga acaaagactg gagaagaaat ttacaggaat |
| 37681 | gttcatttgg tcttaattta aggaagatgt taaataagac ccattacatg tagcatgatg |
| 37741 | tttttactac tcaggccagt tttagtggtc acatcttctt aaggtgtaat aggcagcctc |
| 37801 | agaagttact gtggtcttgc tcactggaaa tgatacacag acagtttaag ggacttgccc |
| 37861 | cagaccacac ggcaagaggt gaatgtcaga acctctttga gtacatttta aaataaggac |
| 37921 | tgaaagttgg aggagggtgg ttatcaaggc tgccttcctt accatagtat tcccagcatt |
| 37981 | aacaaaatcc ttggcatgta attggaattc agatgcttct taaataaatg aaaagcctgt |
| 38041 | tgtagccagc ttacagtttg cattaatgca gattattaaa gtggaagatc ataaatgatt |
| 38101 | ttttattaat atttatgtct ataatcttag gtttggaaaa cattattcat tcataataat |
| 38161 | tttaattata tgttacatta ccacattttt gacttgtagt gtttttagca tagttcagct |
| 38221 | acagtgtagc ttaataaaga atatgatttt ttaaaatagc aatgctatta tatagccttt |
| 38281 | acagaacttc taaaaaatga catgttctct accaccttaa tactgaaact caaatcttat |
| 38341 | tttttgctac gattattcca gctactcttt tttgtctata tttcatttct gcctttttat |
| 38401 | gttgtggtcg aagtaactct gagcttttct catgttgtcc attgttgcat aaaaatcttc |
| 38461 | cagcatctta aagcacagcc tactcacaca aaaaagtgat tgtttgctac agaaaatttc |
| 38521 | ttcaccatcg taattttttg ctacttcaaa ttcagtaagc attcttacac attatattta |
| 38581 | ttttatattc agtgatgaac tatttttata gattccttaa aatttctggt tatttatttg |
| 38641 | ataaggaaac atgtactaga aaaaagtaca acacatatat tgtgagatta attatgacaa |
| 38701 | tttctagaaa gtaacagtct gttcaactca aatgtttata agaaaattct ttctttattt |
| 38761 | atttatctgt gcatttaggc atgccagaag aatccaggga ggatacagaa tttcatcata |
| 38821 | agctaaatat ggttaaaaaa cagtgtttca agaatgatat tcacaaactg gtcctagcag |
| 38881 | ctttgaacag ggtatgttga atataagttt tctgtattta tactattaac taaaatatta |
| 38941 | aatttggaga actaggggcg ctttttcagt ctaagttttc tgttctccgt ttgctatgat |
| 39001 | aggaggaagt catgtggtta gagacataag atgacagtgg ggatgtggga agtgaaaaga |
| 39061 | tatgtactaa gctaagtcca gctaagtgta ttatcaatta tagatgtagg caagattctt |
| 39121 | ttgattgcca gtaacataaa tccactctag tttgctcaac cagaaagaga accaaagagc |
| 39181 | catatatgca gctagacctt gtgagtcatg ctgggtacta tggctgctgt tttctctttc |
| 39241 | tgtcctctgg ctacttgtct ttcttttctg gtctcatagt atatggttta gcccatgaag |
| 39301 | acataccagt gttaacagta aagtcttcgg ctgggcacag tggcccacac ctgtaatccc |
| 39361 | agcactttgg gaggctgagg tgggtggatc acgaggtcag gagttcgaga gcaacctggc |
| 39421 | caacatggtg aaaccctgtc tctactaaaa atacaaaaat taactgggca tggtggcacg |
| 39481 | tgcctgtaat cccagctact caggaggctc aggcaggaga atcgcttgaa cccaggaggc |
| 39541 | agaggttgca gtgagctgag atcacaccac tgcactccag cttgggcgac aagagtgaga |
| 39601 | cttcgtctca aaacaaaaac aaaaacagac aaacaaaaac agtaaagtct tctttgattc |
| 39661 | cctacgctca ttttcattgt tctccggaga aataacctct gaaatgattt ggtatacatt |
| 39721 | gtttccattt tttagcattt acatatccat gttcctacat tataattaaa gtatccataa |
| 39781 | atcatactga gtatgaaaaa gagaagaagg gaattacatt taaattgtgt aatgcaaaaa |
| 39841 | gtattggtgg aattaagaag ttttggaaat tttgcataag atgaattggt tctttattaa |
| 39901 | agatgttaag aataaagaca taattagtgt gaacattttt ataaaaggag gagcctattt |
| 39961 | aaaataatta atggaaatga ttccatgtga tttgatatac tttgatgaat gtcataaatt |
| 40021 | aattaaagtg gcttccagag agatctccct taaaaattca ttttaaattg aactttatac |
| 40081 | tgtcactcac tgcctataat atgtttgagt catttatact caaactttaa tacaatcctt |
| 40141 | gagtatggca agaatttatg ttgtaatggg ttaaatttat cttgagaaat atttgttgaa |
| 40201 | aataagtata tggaaggaag gggttaggca tttagaagat aaataaatat gctttgtact |
| 40261 | cttctctcct gaatctcata agccggttgt tgatggctgt tgtgaaacct tggttctttt |
| 40321 | ctttaaacaa gagacacaca gcagaggaga tgcagcatcg agtaatttat tgcaaaagaa |
| 40381 | aaagaatatt ttgcaagtta agtgaggaat agacacttat accctgacag aattcagggt |
| 40441 | gggcttacta gtaaggatga gacagcgtaa attggcacta ggaagactcc ctttgtggga |
| 40501 | gttgtacatg atttttcata agtgggtggg aagaagtgtt actagtaagc atattctagg |
| 40561 | ttgtcctctg agtgaacatg tgcagtagct gtacatgctt gttcatatat cgcatgtctc |
| 40621 | ataagtatct gaaatctcca cccaggggtg tgtgttttac tattataatg agcaaagggt |
| 40681 | cagtctgagg acaaggaaaa tcaaaatgtg catgctcccc acgctacctg acttcaaact |
| 40741 | atactacaaa gctacagtaa ccaaaacagc atggtactgg taccaaaaaa gagatatagg |
| 40801 | ccaatggaac agaacagagc cctcagaaat aatgccgcat atctacaacc atctgatctt |
| 40861 | tgacaaacct gacaaaaaca agaaatgggg aaacgattcc ctatttaata aatggtgctg |
| 40921 | ggaaaactgg ctagccatat gtagaaagct gaaactggat cccttcctta caccttatac |
| 40981 | aaaaataaat tcaagatggt ttaaagactt aaatgttaga cctaaaacca taaaaaccct |
| 41041 | agaagaaaac ctaagcaata ctattcagga cataggcata ggcaaggcct tcatgtctaa |
| 41101 | aacaccaaaa gcagtggcaa caaaagccaa aattgacaaa tgggatctaa ttaaactaaa |
| 41161 | gagcttctgc acagcaaaag aaactaccat cagagtgaac aggcaaccta cagaatggga |
| 41221 | gaaaattttt gcaatctact catctgacaa agggctaata tctagaatct acaatgaact |
| 41281 | ccaacaaatt tacaagaaag aaaaaacaac cccatcaaaa agtgggcaaa ggatatgagc |
| 41341 | agacacttct caaaagaaga catttatgca gccaacaggc acatgaaaaa atcctcatca |
| 41401 | tcattggcca tcagagaaat gcaaatcaaa accacaatga tataccatct cacaccagtt |
| 41461 | agaatggcga tcattaaaaa gtcaggaaac aacagatgct ggagaggatg tggagaaata |
| 41521 | ggaacacttt tacactgttg gtgggactgt aaactagttc aaccattgtg gaagacagtg |
| 41581 | tggcgattcc tcagggatct agaactagaa ataccatttg acccagccat cctgttagtg |
| 41641 | ggtatatacc caaaggatta taaatcatgc tgctataaag acacttgcac acctatgttt |
| 41701 | attgtggcac tattcacaat agcaaagact tggaaccaac ccaaatgtcc aacaatgata |
| 41761 | gactggatta agaaaatgtg gcacatatac accatggaat actaagcagc cataaaaaat |
| 41821 | gatgagttca tgtcctttgt agggacatgg atggtactca gcaaagtatg ccaaggacaa |
| 41881 | aaaaccaaac accatatgtt ctcactcata agtgggaatt gaacaatgag aacacatgga |
| 41941 | cacaggaagg ggaacatcac actctggggc ctgttgtggg gtggggggag gggggatagc |
| 42001 | atttggagat atacctaatg ttaaatgaca agttactggg tgtagcacac caacatggca |
| 42061 | catgtataca tatgtaacta acctgcacgt tgtgcacatc taccctaaaa cttaaagtat |
| 42121 | aattaaaaaa aaatgtgcat gctccataca ggggcaattc cctactggag atagctttgc |
| 42181 | ttaaatgagc tggactacaa tgcaaatgct gaaacttact atattgacag taagattgcc |
| 42241 | acagttgccg cgtcctgagg acatggttac ttccttttaa tacctatcct gtctcattgt |
| 42301 | gagaggatta acaactgtgc ataaaaccag ttgttctaca tgagcactta ggagggatac |
| 42361 | cagcattgtg aacatagttt aagtacgtag aggagggaac agttaagttt attcatggtg |
| 42421 | agtgttggtg aaaagtggaa gaggtaccaa aacagccgta tagataactg gttccagtta |
| 42481 | gccaacattc tctaaagtta ttagagaagc ctaagtgagg tgtaacctca gcagtcggga |
| 42541 | gccaagagag caagtaagtg ctgtgatgtg gagaaaatca ctttgttcca actgagaaga |
| 42601 | aatggttgag cactgctttt cccccatgcc agtactgacg cacagccttt cacttagcac |
| 42661 | tgattatcga taggggtggg gagttaaggt atggggaaac acaagtaaca atattttatt |
| 42721 | tcaaaaacct ctccactgta attcccctaa tccttcatca tggttgagga aaatggctct |
| 42781 | aaaaaatgag agcaattact gtagctccaa aattctgtga ttgcatgtct tactctgaat |
| 42841 | agcaattaca aagcatcaga ggatttaggt ccaaatattg cagacacaag aaaatgaatt |
| 42901 | acattttaat acatctaaac ttggagagca gagttccaaa taaggtagaa cttgagattc |
| 42961 | aactctgatt tataaagcag agactaagaa gagtatttat aaagcgaatc catgtttgga |
| 43021 | tacataaaag tgcaataaaa ttcaagctga agttaaaatc tctgtctaga acagcgatgt |
| 43081 | tccatttatg cctgatcctt ttagcttttc cacagatgaa gactttgtca cctgttccag |
| 43141 | agagatatat ttgttcatta ttgtttccag agagcaaaat ggaaaataaa ctctgcacat |
| 43201 | tttggccgca tctgtgtttt atatgcggtg acactcctgt tctcttcagt gaggaaatcc |
| 43261 | agtaaagtaa aaccagtctt ctgatgaaat gggcacaaat caaagaactt gtgagcttca |
| 43321 | caaaaacctt gaagcaaaat ataccaagct taaatattga atgtattgat ttcagtagtc |
| 43381 | aaaaacagag ctcatctgca aaagcaacaa caacaatgac aacaacaaat tacatataag |
| 43441 | taaaatttaa aaaaggttta caggatgaat atacagaaaa ctatgaagct taggggtaga |
| 43501 | gaggagtagt tgaatatatg aaaagataca tcttttttga tgaaggacta aatttttaaa |
| 43561 | aatgaaaatt gatctatgtt tatgaaccat tagtaaaaat aacaatagta ttttctggaa |
| 43621 | ctaagtaagc tagtgtaaaa tttgtatgaa aaaattaata catatgagta atcaggagaa |
| 43681 | ttttaaaaaa gagtgttgat tcatatggcc taactccaaa agataatcaa atgtattata |
| 43741 | caattttagt aattataatg gtgtgatact ggcactaggg gagagagatc agtgccacag |
| 43801 | aagggtggct caggaaatag actcaaataa aaatttgtaa tgttatgata ttcctcgact |
| 43861 | gcgggaggaa aatagattac tcagttactt gtgttggaac aactaactct tcagccattt |
| 43921 | gggaaaagca aagaaagctg aatatttatc ttactccttt tgccaaaaca aattacagat |
| 43981 | ggatgagatt taaagtctat aatgaaattg gtatatgtac atattaaaag tttcaactat |
| 44041 | aattgttatt aacgtaaatg acaacggaac accttgttgg agggaaattt ggtaatatct |
| 44101 | atcaaaatta aaatgccatg ttttctgatc agcaatttca ttctaagaat ttttaatgta |
| 44161 | gatatacttg ctcatgtaca taaagattat tagacatgaa tgttcactgt ggcatgattc |
| 44221 | gtaattaaaa atgtggcaac aaactaaatg cttacatggt aatcattcat gctgtttgcc |
| 44281 | agatattttt atttctccac cttgtggtga ttctggcata gtatttagta gttaggttga |
| 44341 | agcatgtgac taattttggt cagggagttg taaatggagg gaatagctgg tgagacatct |
| 44401 | tgcggaatac tgtctcaggt accatcgtac ctgcagtgtt tgagatagta gttgctccat |
| 44461 | cagtagaggt ccctgaggga ctacaacaag cagagtcccc tcctgacctg caacacatat |
| 44521 | gtagcatgag caataatgga acttattatt tttgcaccct ataaaatttt gagaattgtt |
| 44581 | actgcagctt aatttaatct ttcctgacaa tacaatacct atcaatagag actaatcaga |
| 44641 | taaattatac catttccaaa ttgtatcatc ctgcataaat attaaaaaac aatgaggtga |
| 44701 | gattcctaat gtgctgctat ggaaaaatct tcaattttct atgtgtacga atgtatttcc |
| 44761 | caggtattcg ttttttcctt cctgtatgtt gacacattat gcacactttt ggatgaaatt |
| 44821 | aatatatttc caccgcttta tgtcctctcc ctcactttta tcacccaatt gtagcaaagt |
| 44881 | atatgtttgc ttttatagct ttagctatat aaaattttct aatagctaag tttgtggttc |
| 44941 | ctggaattaa gatatctgaa tttaaatcta acactactac ctacagacta tgcaccctgg |
| 45001 | acaagtactt aatgtcttgg ttttgttatg tataaaatgg agataataac tgatttttca |
| 45061 | cttagagttg ttgaatattt tataagataa ttcatgaaaa agtgtcagta taatgcttgg |
| 45121 | cacatagtat gcgctcaata aatgttaatg ttattactat tagatttaaa agtatctttt |
| 45181 | gacccctggc tatagaagat gaggaaatca gagtatttgc acttctaata tctcctgttt |
| 45241 | tcccacctac ttttgttcaa taaattaact ccacattggc agggtagata atatttatat |
| 45301 | tcagctttct aattatgctt tctaagtttg tgtttatctt actcctaccg ttatttggaa |
| 45361 | gcagtcttca tctcaagtcc ttttgttact gttttttcac tcatctttta gttgtctgaa |
| 45421 | tttctttaga aaagtttaat tccctgaata tttaatgttt atattttgtt gttgtttttg |
| 45481 | cctttatatt tggactccta aaaaaagttt tgttggttgt aaaatattgt ttttttttcc |
| 45541 | ttgaagtgtt tgtaggcatt tcactgtgtt ctagtactga atgttttagt ggagaagtct |
| 45601 | gaggccagct tcatttgttt cttggatatt tttgcccagt gctgaaagga ttttttctct |
| 45661 | tgaaatccat ccactttatt agggtatatc tcaatgttag tcctgggata tggtatcctc |
| 45721 | attctagcta tacattcaac ttttttcctg gaaagttttc ttgagttgca tctttatata |
| 45781 | catatacata cacgaataca tatacatggt aaagatatta aggataaaga taggtgcttt |
| 45841 | ataaatacca atttgatata attttggcat gaaaaaaagc ctgtggtgca ttctgagttt |
| 45901 | ggaagtagaa ctggatatac ttacttatta ctattgtttt taatacaaaa atggagacag |
| 45961 | ggtctcacta tgttgcccag gctggtctca aactccttga ctcaagcaat cctgcctcga |
| 46021 | cctcccaaag tgctgagatt acaaatgtga gccaccgtga ccagcctaaa tatctaaatt |
| 46081 | tttctctttc attattttag ctctcttctt tggggctatc agtcatatga tgttggatct |
| 46141 | cttttgcctt aatcttctaa tttttcaaag actaatccag agaccactat ctatcaccat |
| 46201 | gccattactc atagacaagg gacatcattc cacacattta gcttatttgt caaaatcaga |
| 46261 | gatgggattt ataaaagaaa aagaataaag ggagaaaaca aaacacaaat tatctgggaa |
| 46321 | tgcgagtctg ttttcctttc tttggctaat ctacttacct agtagctagg aaagaggcca |
| 46381 | gcatgagtat ttttccattg agctggctag ctcagtcatt ggagaaacca tttcaatatt |
| 46441 | tatggattct ctgaaaaaaa cttggagagt aaaaaagcat aggtggtagg atgctgccat |
| 46501 | ccttctgttg gcatctccag agtttcactt gaaaaacacc tcctaagtag acactgataa |
| 46561 | attgatttta atccctattt gagcaccaat gcaatacatt actatttcaa gatggagcat |
| 46621 | tagataacta aaggaaattc tattgtgggc ttgtaaatat catgatagtc atacctatgt |
| 46681 | aatacatata tgtatatata aatattaatt tttaaacatt tacttagcta ttaaaaatca |
| 46741 | aatgctcata tttaaaatta ttggtttatt aatgcaccat cacatgttat catgtgatac |
| 46801 | tctgaattct ctttttgcat agacaaatca gagattacct gctgcaagtc attagtcatc |
| 46861 | attaacctgt actgaaatgg ttagagccta ggggagttcc aggtaaaagg accagtaatg |
| 46921 | agtagaagct tgttagatgt agagatattg aggacagaga taggtgcttt ataaatatca |
| 46981 | atttgatata attttagcat aaaaataagc ctatgatgct ctttgagctt gggagtagaa |
| 47041 | gtggatatgt aattttcagg gcgtagtata aaatggaaat gcacactcct tgtttgaaaa |
| 47101 | ttattaacga ttttacaagg gtgacaacag agcattgaag caagtgctct gtgagcatga |
| 47161 | agccctgtgt gaaatacaca cctgtgaagc tggccttgcc tgccaaacag gaatgctggt |
| 47221 | actgttaatc aaacagaaag ttcagaatga caatctgact tttttttgta gaagatacta |
| 47281 | aacttttggc cttgaatatc tgaatttaag atactggtat caagccaaaa gaaatttggg |
| 47341 | cttagaactt gaaagattta aacttgtgct ttgacttgtg tgctcacagc tctctgtgtc |
| 47401 | acaatttttt tcatctgtac ttcgggacaa caatagtgtg tcgacatcac aaaggttttg |
| 47461 | ggaggattaa gtaggtgaat acatatgaag ttcttaaaag aatgtctggc actgagtgag |
| 47521 | tgctacctcg gttttggcat cgttattgtg gtcattgcta ttgttattat gacttgaagt |
| 47581 | catattagtg tatgaaatcc catccatgaa tagaagacag aaagaacttt gggcagggtt |
| 47641 | tggaggtaaa agaagacatt gtaaaggaga tgggtaaagg aatgatttta aagactgaga |
| 47701 | ataattaggg gagtacgatg tcatggaaat taagggatga taagcaccaa ttaagttgtt |
| 47761 | attggacttt tttctttgaa tggttcaaat tcagaataat aaggaaagaa gtctgattat |
| 47821 | acgaaattaa agggtagggt gactgtggag gtagtgggag ttgacttttc tgctaataag |
| 47881 | tttagaaata aaggaaaaat ggtagcttga ggaaagagag gagcgattaa gggaaagtct |
| 47941 | ctcgttaact cttgtttttc atctctgagg acagcgctta gcccaaggcc tgacctgtga |
| 48001 | tcattactct ctcaaggttt attccatgga cagagctatc tcatttcatg cttataacaa |
| 48061 | ccctacatta ttagaattgt tttagagatg aggaagctga gacacacacc aaaccagcct |
| 48121 | tccaatttca ctttgcacaa ctttgaattt ctttatattt cttgaataaa agttccactt |
| 48181 | tttaacttac cacttcttag cagtcattgt ctaactgagt aattgttact tcattcattt |
| 48241 | aatggttctc agattcgcat aatttgaacc taaatttaat tggcctccaa gctgatgtgc |
| 48301 | ttacagaaac agtgacagga aacaaaaatg tcaagggaga ctatgtatta ttaagatgat |
| 48361 | aaatgaaatg atgtccaagc tgagcaatta aagtgtgaag tagaaggaca cagggtgaga |
| 48421 | aactgatgct tctcctcagc ctctataaaa aagatactga ataaagataa ttgagaggca |
| 48481 | ttaggggact agactgagaa aggattggaa atctgttcac tgagagtaca gaaatgagga |
| 48541 | agcttggaag gcagaagatt ttggtcaaag acgtctggct tgaagctatt tcagctcttt |
| 48601 | ggattatctg tggtggaggc catcacgtct ttggagtgga ggtaccatga aactagtgtc |
| 48661 | tgcaaaacat catctaaatg aaagcaaaat tcctgagaag gatggcacta taatttttaa |
| 48721 | aagaaaagct atgagttaag cattcatatc aaggtagatg tttggagtgt attgcttgtg |
| 48781 | tgtgaaaagg cagagatgac cagaataaga gttagaggta tgctgcgttt tcttcttggt |
| 48841 | tgatgagtag gatggcctgg acaaagaagt gacctctagt aaaatacctt catagtgtca |
| 48901 | aatcatctgg aggaaattca gattaaagag gttggatgat gtcgtaatta agatcctggg |
| 48961 | cttttaaggt ggacacattt atattcaagt cccaggccca atgcatatta gctctgttac |
| 49021 | ttgagctttt atttctgcat ctttaaagtt tggcaaacct attacatgaa gctgttgagg |
| 49081 | ggataaatga aataacgcat gcaaagcact tgcagtaaag actaattatc aatattttat |
| 49141 | ttgttaagag gcagcattgc gttttactgg tcaagtatgt agactctggg gtgaaacata |
| 49201 | tttggtttgg tttcatctct gcaatttata gtttgtgtag actttgagaa tagttctcaa |
| 49261 | tcattctaac cctcagtgaa ttcatcttct aatgggagtg atatcagtat ggatttcatg |
| 49321 | agattatgaa aagaaaatgc ctacaaagta tttattacaa tgcctggcac agaacaagct |
| 49381 | ctccttaatt gtaaaaatgc taactcttat tcttcataat aaataaaagt aattaatgtt |
| 49441 | atagaaaaca aaatcaagga tactgattta tatttggatt acttgattta tattttgtca |
| 49501 | gtctataact ggtcttaact aaggtaagta ttaagatctc atttttaaca gcgagtattc |
| 49561 | ttttgatttt agttcattgg aaatcctggg attcagaaat gtggattaaa agtaatttct |
| 49621 | tctattgtac attttcctga tgcattagag atgttatccc tggaaggtgc tatggattca |
| 49681 | gtgcttcaca cactgcagat gtatccagat gaccaaggtc agtacaattt gaattcagga |
| 49741 | tttagaatag atttttgtag ggcattagct ggtgactgga tgtctttaaa tatttttctt |
| 49801 | cagttttgag atttaaaaca attctttttt tttattttcc tagaaattca gtgtctgggt |
| 49861 | ttaagtctta taggatactt gattacaaag aagaatgtgt tcataggaac tggacatctg |
| 49921 | ctggcaaaaa ttctggtttc cagcttatac cgatttaagg atgttgctga aatacagact |
| 49981 | aaagtatgtg cattatcttg gaaagaattt gggaacttgt gcgaatttca cttttggagc |
| 50041 | agtttgtgta attcccactt tgcatgaatg gggtattcta gttaatggaa aaccatttat |
| 50101 | ccttttgtag tattttaatt atacaagcaa agaaaattgg attgaatctc taaagatcca |
| 50161 | gtgtttcatt atgaaatctc taaagtcagc atggttattc accatttatc ttgcccataa |
| 50221 | aagttcagag aatgtgctaa gaaatcccag ctagctgagt ttattcgctt agattttaga |
| 50281 | taaatagaat ttataaatat tccaaagttt gtcactctct gggttttatt gcaggttgct |
| 50341 | tacctttagt aattttgctt gttgattttt ttccttgcag tgaaaaaatg tttttaacat |
| 50401 | ttttcatcaa gcaaaattta aaacatgata tataataact gtctttgtaa ggaattcaag |
| 50461 | atactggcct agagttagtt cacgggagat taagaataaa tttgttttgt tttgtttttt |
| 50521 | aattgtagca aaacaaatag tttttcttca agagtttctg ccttggttgt ggagtttgca |
| 50581 | actttcataa actacaaagg aatttttttt ttttttttgg agacagggtc tcactctgtc |
| 50641 | acccaggctg gagtgtagtg gcagatttca gctcactaca acagctgctt cccgagctca |
| 50701 | agtgattctc ttgcctcagc ctcctgagta gctcagacta caggcatgca ctcccatgcc |
| 50761 | tggctaattt ttgtattttt tgtagagatg aggtttcacc atatttccca ggctggtctc |
| 50821 | aaactccctg gtctcaagca atctgtcctg ctcagcctcc caaagtgctg ggattacagg |
| 50881 | tgtgagccaa ggtgcccagc tgactcagga aatatttttt gtaactggca gcattgacca |
| 50941 | ggaataaaaa tacctggtct ctaatctttg cacagacatt atcagtaaat gagagaatat |
| 51001 | gtgtaaagtt ttttaaaaaa ttataaagtt atgaacatac aaaattctta gattaataac |
| 51061 | aacaatgtgt tttataactg cttttcataa tgtgcctcag gctaggctga ttaaaccaag |
| 51121 | ataggattga ttaaaagtaa tcttagggaa agggaaggat tttgtgccgg tatggaactc |
| 51181 | tcagttactc tggattaatt catctaggca taaattttag aatctctata gtagagttta |
| 51241 | tgaactaaat ctggcctgcc aacatatttt atttgtccag ttcagggttt tgctttgttt |
| 51301 | tttgagacag agtctcactc tgttgcccag gctgtagtgc agtggcgtag tctcagctca |
| 51361 | catcaccctc cgcctcctgg gttcaagcaa ttctcctgcc tcagcctccc tagtagctag |
| 51421 | gactacaagt atgcaccacc atgctcagct aatgtttgta tttttagctg agatggggtt |
| 51481 | tcgccgtgtt ggccaggctg gtctcaaact cctgacctca agtgattcac tcacctcgga |
| 51541 | ctcccaaagt gttgggatta caggcatgag ccactgcacc cggccttcag ttcagtgttt |
| 51601 | aaaagttttt aattcgaatg acgtactttc tgcacatttg catggcctgc tctgctgtag |
| 51661 | cattcacttg ttttcagaga cctctgctct agaggcaggt ggatcacctg tccctcagac |
| 51721 | atacataaat taaggctact ttgcttatca aatattagta ttcctagata ctcagcatca |
| 51781 | taagagttcg aagtaataat tttaatattt agatgacgta agttaattta aaattttttt |
| 51841 | gagatggggt ctcactctat ggcccaggct agagtgcagt ggcacaatct cggttcactg |
| 51901 | caacctctgc ctcctgggcc catcctcctg ggtgggctca agtgatccac ctcagcctcc |
| 51961 | tgagtagctg ggactacagg tgcatgcacg ggtaatttta aaatattttt tataggcaca |
| 52021 | agattttgcc atattgtgca ggctggtctt gaattcctgg gctcaagcaa tcccacagtg |
| 52081 | ctgagattac aggtgtgagc catggtgtct agccaatttt attaatatgt aatattagag |
| 52141 | gtaataaaat attaaaaagt taagatgatc cttggtggct ttacccaacc taaataatac |
| 52201 | taaagtcaaa agcccaatct ttcattaaaa catcacatga gtgaagagga cagactctgg |
| 52261 | ggatgtgctt aaggtggttc taaaaaagta acggtcttct ttataaataa cttattatta |
| 52321 | gaatgtaatc ctcagagtgc cctcagcgct tctcaactac actcaacata aatgaaatct |
| 52381 | aggagtccac actagccttt ctgagataaa catttcggaa gacagcgcaa aaagctgggg |
| 52441 | gatatgctag gctctctaga gaacctactg ttcaatatta taatacaaat ttttactcta |
| 52501 | ttgacctgtt tggatgtgta gttctgctga tccaaccgct ttaatcctgt ttaatatctg |
| 52561 | ggtttcatcc tataactatg gctttagaca agcatctttg aaaaccaaat ttgagggtat |
| 52621 | tagttctttt tcctgctttg ctactgaatg gtttgttaac tagcatttta ttctctgtgc |
| 52681 | ctgctatatt tcttagtcat gagagagaga gggagtattt atttacagga taaatacttt |
| 52741 | aaagcaccaa cccaatatat ctatagttaa atgaacatcc taggtattgt ttcatataca |
| 52801 | aactctctct gctttatact gtttattcat tttgcctgta attgcttatt ttattatttt |
| 52861 | ttttcttata cttttaggga tttcagacaa tcttagcaat cctcaaattg tcagcatctt |
| 52921 | tttctaagct gctggtgcat cattcatttg acttagtaat attccatcaa atgtcttcca |
| 52981 | atatcatgga acaaaaggat caacaggtac agtgtttttc acttgcatcc taaatgttat |
| 53041 | gtatttatct gactctaatt ctcatttcca ctctttttag tttctaaacc tctgttgcaa |
| 53101 | gtgttttgca aaagtagcta tggatgatta cttaaaaaat gtgatgctag agagagcgtg |
| 53161 | tgatcagaat aacagcatca tggttgaatg cttgcttcta ttgggagcag atgccaatca |
| 53221 | agcaaaggag ggatcttctt taatttgtca ggtaaatatt caaggcctca cttttgtctt |
| 53281 | tgctcagtat tcttatagaa tgtaagagcc ctgccattgt gtatctctta cttatatcat |
| 53341 | attattcttc actacagaaa tttaccagtt tattgcaatt gtttgtgtct tgtagtagat |
| 53401 | ttatagaatt ccagaagtaa tagggtcctt taggtgttat ccagtctaat ctttcatttc |
| 53461 | atctgtttac ttatcttgtt aagttgataa ataacttttc aaatgtgtcc cttagtaggc |
| 53521 | atctctacaa cttagtctcc agatacactc cacataacac atagttctaa tgttttgata |
| 53581 | atttttaaac catttttttc catggtttta gtttctttgc ctagaaagtt ctcccctgag |
| 53641 | ggctaccaca catggctatg caggctgtgg atggcacact tttgtcggtg ccattcacag |
| 53701 | tgacatgagt tgctgttggc caaagttgtg taacactggt ctttctttcc ttctctcttc |
| 53761 | cctcctgaac catgtaaaca tatatctatc tgattgttct gctctccctt caaaatataa |
| 53821 | ttcaaattat ctttctttaa agccctcccc atacctccaa acctccaaac aaaattaaga |
| 53881 | tttacttctt ttgtcagtct atgaaaatat atacatatct cttgtatact tggtgagttg |
| 53941 | tgtgaaaata acagtgtaca gtgttcatct ttgtatcatt cagaatatcg agctcattgc |
| 54001 | tttacatatg gtgtgtattc aataaatact aggttcattg cttatatttc agatttgtat |
| 54061 | tatttgtata agtgttagag tttatactag cattcaggta gcactatgtc tattttctag |
| 54121 | aaatttaata tttctaacaa agcaattatg tagtgattta atacacatta ttaaataatc |
| 54181 | aataaagtac tatgtttgcc aatagtttac tttttaaacc ttactgtatt taatatccct |
| 54241 | actgtattta atatcccact tgcctatgga ttgaaatcaa tttgttgact gttaagatta |
| 54301 | agttaatact aattagtaat caacataaaa agaaaaagaa tttgtaaccc attttcatgc |
| 54361 | attacgttta tgaattaaaa tcacataaac aatctaatta tttaaattta gtcaaatttc |
| 54421 | ttttaagcaa gcaacaatta aaatagttgc tccgctttac taaagataat taaatttttc |
| 54481 | catcaataat ttaatacatt tttactgtgc atcttttgca tgcagattat tgcattaatt |
| 54541 | ttaattgaaa ataccgaaga actaaaaaga aacttcccct tctaagtcca cattaaggaa |
| 54601 | acaacatacc taaaagcacc tgatacaact gtactacatt ccccacagga aatcatttct |
| 54661 | actattcttt caatttatcc aaatctttct acccaacagg atttttactt tattcctctt |
| 54721 | tccatattct tttggacttc atatgcttag ttttatcttt tctttttaaa acgaaatctt |
| 54781 | aaatccaagg attatgtatt aggtttaaag aatttatccc agttgtcaga ggttatttat |
| 54841 | atctagcaaa caataactgc tgattaaatc ttgtggatga gtttgtcgta tgtaccttat |
| 54901 | ttgtgccaga gcaaaataag gtaatcagga ctatttattc atttaccaag aggttacata |
| 54961 | ttgaaggact atctagagca agggtggagt tgtgttagac tttctgcaca gaatttgata |
| 55021 | atggaatgta catgattggt agagaagaat atggaagttt aatactgggt atgcaaatgc |
| 55081 | atggataaaa acctcaaggt aaaactcatc aaatcacagt ggaaaaagta tagtgaagtc |
| 55141 | tgaataaaaa taataagagg ctgggcatgg tggctcacat ctgtaatccc aacactttgg |
| 55201 | gatgttgagg tgggaggatc acttgagcca ggagttcgag accaacttga gaaacatagt |
| 55261 | gagactccat ttctacaaaa caaaccaaca agcaaaaaac catgtatgat ggcacacaca |
| 55321 | tgtagtccta gcttcatgca gggtggctca tgcctgtaat cccagggctt tgggaagtca |
| 55381 | aggcgggagg atcatttgag cccaggagtt caagaccagc ctgggcaaca tagttagacc |
| 55441 | cccatgtcta caaaaagtca aaaaattagc tgggtatggt ggtacctgct tatagtccca |
| 55501 | gctacttggg agtctgaggt gggaggatga cttgagcctg ggaggttgag gctgcagtta |
| 55561 | gctgagattg caccattgca ctccagccta ggcaacagag ccagaccctg ttaaaataaa |
| 55621 | ataaaataaa ataaaataaa ataaaataaa ataatataat aaggctgagg tgggaggatc |
| 55681 | acttgagcct aggaggtcaa ggctgcagga gctaagattg tgccactgta cagcagcctt |
| 55741 | ggtgacagag ggagactctg tctcaaaacc aaccggtcgg gtgcggtggc tcacgcctgt |
| 55801 | aatcccagca ctttgggagg ccgaggtggg tggatcatga ggtcaggaga tcgagaccat |
| 55861 | cctggctaac ccggtgaaac cttgtttcta ctaaaaaaat acaaaaaatt agccaggcgt |
| 55921 | ggtggcaggt gcctgtagtc ccagctactt gggaggctga ggcaggagaa tggcgtgaac |
| 55981 | ctggaaggcg gagcttgcag tgagcctaga tcgcgccact gcactgcagc ctgggcgaca |
| 56041 | gagtgagact ccgtctcaaa aaaaaaaaaa acaaaaaacg aaccaaccaa ccaaccaaca |
| 56101 | aaacaaacaa acaaaaaacc aacaaaacca aacacttcta tcatgctcat taccacctgg |
| 56161 | gcactgctcc aaatacttta cacaatttaa tccttacgac aacctacgaa aaggtccagt |
| 56221 | aggttctaat gttattccca ttgtgcaagt gagaagctga ggcactgagg gtttaaataa |
| 56281 | cttgcctaag aacaagctcc tggtaacagt gtgaaatctg cctccacagt gcctgcttta |
| 56341 | atttcttggc tacacagcag attcatggta gtggtggtag tggtgttcat tttctctaaa |
| 56401 | ataacagttt gaataatttg gttttgataa tgcactgcat ttattataaa ttagatgatc |
| 56461 | agagaaagat tgcagggata agaaattatg cttttgataa tctttagtta tattcttaat |
| 56521 | tttcttcatt attatttaaa tgtaaaaata aatatctgtg agcagtagta ttttcctgtc |
| 56581 | atgaagctga aattactttc ataaatatgt gtgaatattc taaagagaat gactctgtag |
| 56641 | gatttaaaga aattaattct tatttttgct ggcatttatt tattttatca gattcacttt |
| 56701 | ctcatatatg tctctcttca tggcaccata tgcctaaagt cagcttggat agtttggatc |
| 56761 | ctccaaggaa aattccttcc acaaacatgt gcagcacaca gtgctagata attaatagag |
| 56821 | aatataaaat gggtttcctg tttcaagatg gtttgtaggt ctgtatgtgt agggcattga |
| 56881 | caagagagta aaacataaat caccttagta caaagtaagg agtgaatggc atatcttaga |
| 56941 | gaaaaaaaag ttactgggct ataagagaag gcatttgtga gtttttccct ccctccccgc |
| 57001 | ttcccttccc ttcccttccc ttcccttccc ttcccttccc ttcccttccc ttcccttccc |
| 57061 | ttcccttccc ttcccttctc ttccctcccc tccccttctc ttccctcccc tcccctcccc |
| 57121 | tcccctcccc tcccttctcc tttctcttcc ccttcccctt cctcttcccc ttccccttct |
| 57181 | ccttccttcc ttccttcctt cctcttccac ctgccttcct tttaattttg ctatgagccc |
| 57241 | ttaaagagga ttttagtaat ttgctactta aattaaatat atttgctaga tgttgtgcta |
| 57301 | ggcttcagga atacaagttg gattgcagta atgtaaagcc ctttgcattc tagcaagaaa |
| 57361 | acagatgggt atgtatgttt gctcagtgct acattaaatg aaatggatgg gagccgggag |
| 57421 | gagaaatggt gtgtttggcc tgagaggtta gtagcaagga cttctctgca agaaagtttg |
| 57481 | aagccaattc ttcaagaatg aacacctttt tgctgggtga aaagtagagg aaggcatttg |
| 57541 | gggtaataga aatagcataa aaggtaatga ggtttgaaaa attacatgct gtgtttggaa |
| 57601 | gaatgtcctg gagcagcagc gttttagaag gtttttaaag acgatggtga cttgatcaga |
| 57661 | gctctgtagt gctttgagga tgggttgaag gtgggcgtac ttggagactg gtgggcattt |
| 57721 | aattggtgcc ttccaaccac ataaatgaat gtcccctcaa atcccttgga aacactttaa |
| 57781 | ttctagaaaa ttcaaaaatt gtccccaaca tctttttcct ctgagttggt accctggatc |
| 57841 | tttgggtctt cttttctttc cttttttgat gttttatttt gggtaatgaa agtcacacag |
| 57901 | gttttgaagc cagcagattt ggcttcaaat ccaagtctca gttgcttgct agctgtaagg |
| 57961 | gacaaattat atatcttttc taaatactca tctataaaat gggagtaata attgctatgg |
| 58021 | cataggattt ttttaaaaaa aagattagaa atcatgtgtg tacagaattt agcacagtaa |
| 58081 | ctgatggata ttatttctat tacctgttat cttggtcttc tagttgatag ctccttgcta |
| 58141 | gcgtctagct cctttccata gctcttcctg agtagggcca gcatgcagtg ccacagcttg |
| 58201 | ctaaggcttc tcctggattg ctgagttgtt ctagtttttg tggcacctca catgctaacc |
| 58261 | caccctgaac acatgctctg aaaacataac atttagagga aggttgaaga ctgagagaca |
| 58321 | aggtatatct ttgaggaaat tcagatgctt gtcttgagga gctcaggaaa gctagacacg |
| 58381 | agtaatgact gtcgtttgtg tgtggcatta ataaatttta caatagctat gtccccattt |
| 58441 | agttattcta tgtcacaaat aaaggcagga cagtagtatt tactgtgtta aggtactggt |
| 58501 | ttcccaggta tcttacagtg agaagacaga agctcagaaa gtgtaagcaa tgtgcatatt |
| 58561 | tggtggagtc tggatgtaaa cagagatctt gatgccaagc ctgtggagct ttgtctccat |
| 58621 | ataatgttgt ctctttcata ataactgact gtcatgtggc agattattca tgctattctg |
| 58681 | acattgatgg cattaatatc atcttatttt cccaatctat tcaaggatca gttttgcctt |
| 58741 | attttatttt gtttcattcc aaattggaga tgtagagaaa aatcacatga agtttgattt |
| 58801 | gccagtctcc taaaaggaag aaaaatgtag atttttaata tacttaattt tttgtcttta |
| 58861 | ataggtatgt gagaaagaga gcagtcccaa attggtggaa ctcttactga atagtggatc |
| 58921 | tcgtgaacaa gatgtacgaa aagcgttgac gataagcatt gggaaaggtg acagccagat |
| 58981 | catcagcttg ctcttaagga ggctggccct ggatgtggcc aacaatagca tttgccttgg |
| 59041 | aggattttgt ataggaaaag ttgaaccttc ttggcttggt cctttatttc cagataagac |
| 59101 | ttctaattta aggaaacaaa caagtaagta acaaggagaa tattttttac aattcttatt |
| 59161 | tttaatagta tttttttaag tcactagtct tttagtggtt attcatgcca gtttgaggga |
| 59221 | ccttaagcca aagatattgc aaaggtttgg attttttttt tttttggcta tgaaatactt |
| 59281 | caaaatgaca tttaagttct ttatgagata gcaaatagtt atttataaaa atagagcaaa |
| 59341 | atagtggaag ctttttgaag gggtactttt taatatatat tttttattat taaagtaaga |
| 59401 | tatccctgtt tttaaaggaa atataaaatt ataaaaaaga aaataaaaat aacttatttt |
| 59461 | atctcttata agtaattaat atggatattt ttcctaactt tttatatgct tacatgtacc |
| 59521 | tatgcattca aatgtatgta aaagcataca cacatattta tttggcattt ttaacttaga |
| 59581 | atatacttta tatttcaatt gataatgcat tttctttata ctttcaagct catgtgtatt |
| 59641 | ttgtacatat tatgtgtatt gatggtaagt taccatcttc tgacactatt tttatctttt |
| 59701 | gagctctctc atttgttcac actaaatgtg tttttagcgt gaaagctccc agctttccct |
| 59761 | gtgttaactt agtcccatgc ccatctcctt ccccatggtc atcaaactcc atgaatcaac |
| 59821 | accttaagga ccatcttgca agtaacatgt ttgcttctct catttttatg atgcactcac |
| 59881 | tagcaaaaca ccagttttgg tcagtctacc agtctacttt ttccctcagt ttcaccaaga |
| 59941 | aaactgagtg ctgctagaga aaagtaccca tccatgcaat ttggtgcctt tatacatcaa |
| 60001 | ggtttccaac cgctcagtag gctccaaaag ttccaatcag gctgaatttt cctcggtttc |
| 60061 | tcaaacactt cgtgtaccct tacttccagt ctttttccag tgttactctc tctctaccta |
| 60121 | gctctaaatt ctctcttcac ctggctgtct cttcattctt cctgtctcag tgctatcacc |
| 60181 | agtctggaag gttctcttac atgaccctat agcactttat ttctcacata tactaccatt |
| 60241 | caccacatta tataatttaa tttttcattt ttataatcta ctttttggta aattgttagt |
| 60301 | accatgaagt caatgtcaat tttgttcatg gttgtaacct taccattgat actagtgttt |
| 60361 | tgcacatagt agattatcat ttagaattaa gtattcaata ttggcaaaaa ataaaaattg |
| 60421 | tgtaatacat tatgttgata agcatgtgtg gaaacatgct tcatatattg atatgaattt |
| 60481 | aaattgtcct cttttgagga caatttggca atatctacta atatttttaa tatatgtaca |
| 60541 | tacttttttg acctcacaat gtactgttag gaatctatga tacagacatt ctcaatgtgc |
| 60601 | acaaaaatta tgtacaaaaa tgcacattaa aacattgttt ataatagcaa aagagtagga |
| 60661 | aaaaaaccta agtattcccc aaaaggaact attcaaataa ataatggtac atacatgttg |
| 60721 | tggaatgctt tgcaatcatt gaggaaaaaa aaacgtggag caaatttaat gtcctgataa |
| 60781 | agattacatt actccgtggc aaaaaaaagg gcacagacag tgtttttact atgctaatgt |
| 60841 | tgatgaaaat gcaactggaa tatgatagtt ataaaagttt gaatatgaaa taaaaccctc |
| 60901 | cagaaatggg ttccctggtt gtctctgggt ctttggaaat tactgagaca tggttagatc |
| 60961 | ccatgtttca ttacttaaac tagtcttatg ccaaaaacct gcttacttta atcttcaata |
| 61021 | tccgatggag aggaattgtg ggcccattgg agagggacag agggagattt atcattcact |
| 61081 | atattctctt tgttctgtct ggagttttta ccattgacac atcttaccca gttaaaaaaa |
| 61141 | catagaattg tcatttgatt aattggaggg tataaccatg atttcactgg cagctggtct |
| 61201 | gagtaaagaa cactttgggt catagctttc aaacattttt caggtagtat ttgcctaagt |
| 61261 | gacatatttg tgtgtgagct catcctaccg ggcttcggga taatttccca tatcataaca |
| 61321 | tattactctg gaaaaaggaa ccatttgggt atatgggtat agtgtaagcc atagtatcag |
| 61381 | ttgccttctt ggggtttatc atatgggtcc accacatatt tacagtagga atagatgtag |
| 61441 | atacatgagc atacttcact ctgctactat aattattgct attcctactg ttgtcaaagt |
| 61501 | cttttagctg attatctaca cttcacaggg gtaatatcaa atgatcccgc accatgctct |
| 61561 | gagccccagg gtttattttc ctttttacag taggaggcct aaccagcatt gtattagcca |
| 61621 | actcatcact ggatattgta ctacatgata cagcagtatc atgtagtaat atttgaagtc |
| 61681 | attacagtag taatatttga agaaatcttc ctggctgtat gtagaacaat gaggactcag |
| 61741 | ccaacttatt cttcatagta gagctaatac ataatgtaat gaagttgtga gaagaatgtt |
| 61801 | aactttgaaa ttccatcagg tttcccaata gtcataatga atcactcagc aaactttata |
| 61861 | aaaataacaa gatcctttat ttagcagttt atgtgttcta tgcattgtgc taaacatttt |
| 61921 | atatgcatca tttcaattac tctttttcat caccatatac tgtatttatt atcatcttca |
| 61981 | ttttccaggt gaggacactg atacccaggg agctcatata actcacaaat ggcatttcta |
| 62041 | tgacttgaac acaggcctgt ctggcttcaa agcctaggcc ttttcctgaa taaagttagt |
| 62101 | tccatagaga ttcagttttg ctgtctacat gaaagcattg tgtacatggt tatgtttttt |
| 62161 | taaaaaatat atgatctgcc acctgttaat tattcaggat cactagtgta aggtgacttt |
| 62221 | gaaaggaaaa atagaaatat tctccagaag catagcaata cgtaagaact ttggtcctat |
| 62281 | gtatgtttat ttttgcataa ttgttgattt ctaagttgct ggtgtatctc ttattttcag |
| 62341 | atatagcatc tacactagca agaatggtga tcagatatca gatgaaaagt gctgtggaag |
| 62401 | aagcaacagc ctcaggcagc gatggaaatt tttctgaaga tgtgctgtct aaatttgatg |
| 62461 | aatggacctt tattcctgac tcttctatgg acagtgtgtt tgctcaaagt gatgacctgg |
| 62521 | atagtgaagg tatttattat aaaaaaaaac cctttatgct ttatatttac acactgacat |
| 62581 | tgaacaatag gacccaagac aaaaacctga cctaaatcat ctggaaaaac ttgagtagaa |
| 62641 | atgtgtttat tatcgcaaac agttaagttt actaattttg gttaaagtga tgggtcaagg |
| 62701 | aagtgtgtct ctgtgcttct aaatgttata ctaattggtt aatggttaat attccaggaa |
| 62761 | acaaactctg actagactgg aacgagattc cacgctctgt cattgactag atcctttcgt |
| 62821 | ggcttgtgta agccccttaa ccttgttaaa ggtagtaatg tcgactttgc agggttatac |
| 62881 | ataataatta gaaaaaatgt atgtaaaatg tctgcaacaa tgcttggcaa acaggaagcg |
| 62941 | cttaataaaa aaggttttta tctttactat agcttaaaac aatattaata ttttaatagc |
| 63001 | tcacttgaga taacttttta aaaaattaat atggtgaaat atataatgac aatgattagg |
| 63061 | gctgatgtat ttagcattag cagtttggta aaaatggagt gaggggcttt cttattaata |
| 63121 | tagtatgatt gaaaacactg ggtgatagaa taaggatatt tgagagggca aaaaatgaga |
| 63181 | gttgttccaa aatattgtgt ctcaagtcaa accattttta aaaatcaagt gtagtgattt |
| 63241 | atatacatat ataatttata taaaataaaa tgcattcact ttaagtatat attttcatgc |
| 63301 | gctttgaaaa ttaacatatt catgtggtca ttgtcgctat taagttatgc aatattttca |
| 63361 | ttatccaaaa aagtttcttc atgcctcttc actgaaaaca tccccttccc ctggcccccc |
| 63421 | tgacccttag caatcatttg cttcctgaca atgtagatta atgttttctc tagttttata |
| 63481 | tacataggat catacagtga gtactctttt gtgtctgttt tccaaaatga ttgtactatg |
| 63541 | ttctaccccc accagcagta catgagcatt ctggttgctc tacatccttg tcagtacttg |
| 63601 | gtattttcaa gttactttta gctgttctag tggaggttta attcatttag atgtaatttt |
| 63661 | catttccctg atgactaatt atgtagagga tgttttcatg ttcttattgg ccattcttat |
| 63721 | ttttgtgtga agtgttgaag tattttgctt ttaattgggt tgtcttatta tataagagtt |
| 63781 | ctttgtatat tctagataga agttgtgaca ggtatatgta ttgcatattt ttttcccagt |
| 63841 | catagcttgt cttttcattt tactaattct atttttaaca aaacagaggt tttaaatctt |
| 63901 | ggtgaaatgc agttttccag tttttttctt ttatggtttg tgctttttgt atcccactta |
| 63961 | agaaaccttt tcttagccta aatttgtgaa tattttctcc catattttct cttagaagtg |
| 64021 | ttaaaatctc agctttggca tttaggtcta aaacatttta agttgatttt tgtgtgtggt |
| 64081 | gtgtcaatga agagttgaca tttatttctt tctgtatgga tatccagttg ttccaacatt |
| 64141 | acttgttgaa aatattatat aattcctcat tgaattaatg gaagctttgt tctctttaat |
| 64201 | tgactattta ggtatggttc tattttagca ttatttattc tgtgccactg atttatacct |
| 64261 | tattcttatg ccaataccac actgtcttga ttactctagc ttaatagcag ttcttgaaat |
| 64321 | cagatagtgt aagtcctctg gtgttctttt aaaaaaaatt gttcttatta ttctaggttc |
| 64381 | tttgcatttc catataaatt tttaatcaac taactttatg ctgggatttt tattgtaatt |
| 64441 | aagtccatat ttagatttta tatagaattt atttataaat taaatcatat tacctatgat |
| 64501 | tttaatgtaa tctataatat ataattaata cataaataat aatttatata gattatatct |
| 64561 | ataaattaat ttgagggaac taatatatta ataatgagtc ttttgacata ttaatgtgat |
| 64621 | atatagttca attagtcttt taaaatttct aacagtgtgt tgtattggat gttttctgat |
| 64681 | actattatag atggtattgt atttgaattc taatttccag tagttcactg ttgatatata |
| 64741 | gaaacataat tcatttattt gtacatgaat tttgtatcct gtaaacttac taagctcact |
| 64801 | tacgtgttcc agtaccttat tatagattct acaggatttt ctttgttcac aattatacca |
| 64861 | tttggtaata aagacagatt tgctttttct tttctaatat ttatgtcttc tttttttttc |
| 64921 | ttgtcctatt gcactggcta ggacctccag tactatgttg aatagaattg gtcagactgg |
| 64981 | gcatcattcc ctggtttcca aacttagagg aaaaacatac agtctctaat cactaattat |
| 65041 | gacattatct gtagtttttc atagatgccc ttcatcaaat tgaagaagct tcttcctagt |
| 65101 | catttttgga gagccttttt ttatcattaa taggtgttga aaaatgctct tcagcatctg |
| 65161 | ctgaggtatt catatttttc acctttattt tgttaatatg gtcaaataca ctgactgatt |
| 65221 | ttctaatgtt aaaccaactt gcattcctgg agtaaatctc acttggttat ggtacattat |
| 65281 | cctttttata tagtattaaa ttttgttttc taaattttgt taagaatttt gcatctgtga |
| 65341 | gagatattag tctgtagttt tatacttcct tgtaattatg ggagtaatgc tggcctcttg |
| 65401 | aaatgaattg ggaagtgttt cccattccac aattttctgg aagaatttgt gtaaaggtat |
| 65461 | tttcttctta aatgtctgat agacttcacc agcaaaggcc atctaggttt taaatttttg |
| 65521 | tgaggaggag atttagaatt atgaatttaa taactttgat agatgtatga ttattttaat |
| 65581 | tttcttttac ttcttaagtc agttttagta atgtgtaact ttcaaggaat atgcccattt |
| 65641 | catataagtt gccaaattta tttgtttaat gttacttata gcaattattt aattatcctg |
| 65701 | ttagtgattt aattatcctg ttaatctctt caaagaatca gatttttgtt atattgattt |
| 65761 | tctctattgt ttgtgttact ttctcacgga cttttgttct tatctttatt gttccctttt |
| 65821 | ccttacttat ttttatttta attgactctt tttatagatt cttaatttgg aagcttagaa |
| 65881 | cactggtttt tagaaccttc ttattttgta acagaaacat ttatttaagg ctgtatatgt |
| 65941 | ccttataaat atcactttca ctgcatccca atattttgat gtgtcctctt tctattcatt |
| 66001 | taaaaaaatt taatttccca aatgtccaac aatgatagac tggattaaga aaatgtggca |
| 66061 | catgtacacc atggaatact atgcagccat aaaaaatgat gagttcatgt cctttgtagg |
| 66121 | gacatggatg aagctggaaa ccatcgttct cagcaaacta ttgcaaggac aaaaaaccaa |
| 66181 | acaccgcatg ttctcactca taggtgggaa ttgaacaatg agaacacttg gacacaggaa |
| 66241 | ggggaacatc acacaccagg gcctgttgtg gggtgggggg aagggggagg gatagcatta |
| 66301 | ggagatacac ctaatgtaaa tgaggagtta atgggtgcag cacaccagca tggcacatgt |
| 66361 | atacatatgt aacaaacctg catgttgtgc atgtgtaccc tagaacttaa agtataataa |
| 66421 | aatatatata tatatatata aatttaattt cctttgtgac ttctttgatt cacgagttat |
| 66481 | ttagaaggct gtcatttatt ttccaaatat ttggagattt tctggataac tttctggtta |
| 66541 | ttggtttcag tttgactttt ttgtgctcag ataacctact ctgatgattt ctatcctttt |
| 66601 | atatttattg aaacatgctt tatggattat catatggtct gtcttggtga tgtgccatgt |
| 66661 | gcaactgaaa agaaagtata ttcagctgtt attgggtcaa atcaataggc caaattgttt |
| 66721 | ggtaggattt ctcaagtttt ctgtgacttc actgactttt ctttctattt attgtatttg |
| 66781 | atagagaaat attgaagttg taattgagga tttgttggtt gccttttcag ttttatctgt |
| 66841 | ttatgctttg tatattttga agctctgttg tttgttgtgc aaacattaag gactatataa |
| 66901 | caaaggacta tatataaaaa taatttttta aataaagtaa ccactttata ttaatctaag |
| 66961 | tttgcagtta gtatcatttt cttcctctgg aagaattccc ttttacattt ctagcgcaga |
| 67021 | tcttccagaa acccatatgc agattactgg agcacttttt ccataactgc cccagaactc |
| 67081 | ccagctatct cagtctccat aaatgttgat ctctatctcc tcatctcagg aaagccatga |
| 67141 | gactgtttaa atttcttctt atgtatatgg cccttaattt gtctctaagc agaaggccag |
| 67201 | agtgatcata gggctcacat aatttgtttc ccttctttcg ggtattacat agtcctatgc |
| 67261 | tgtctgttgc ccagtgtctg tatccagtca tttaatatat ttatccagtt ttcttgttgt |
| 67321 | ttatgtgagt aggttaagtt cacatttgtt actctatcat gcctgaaaga actctcttca |
| 67381 | agtatttttc aagaaaacat tagaaatttt aacataaaat aattataaaa tgcactatgc |
| 67441 | tgcttgtaca aataatgtgc taatagacat ttcacacaca tataaagtgg gataattatg |
| 67501 | ataatttaat aatctttata attatgtaat tgttactaaa ttataaatta tattttatta |
| 67561 | aaattcttaa gcattaaaat aatttaaact atagaattaa ttaaacttta gcaatgaacc |
| 67621 | aggcaggaac tgtttatatc aggatttata ctgaatatta taagggtggt atttggatat |
| 67681 | acagatttta taatttggtt ttgacataaa atgaattctt tctttttcta agataaagag |
| 67741 | taggggaatt taaaatcata attaattttt cacaaatgca ttcacattat ttatctttaa |
| 67801 | tatgattttt ttattgatct ttgttttctt gtaaatttat tattttgaaa gtactgcctt |
| 67861 | ttccttattt tataaaacaa ttattgccag ccaaatttat tgtgtttatt ttaataccat |
| 67921 | tccataaaag aaaccatgaa acatgaaatt caaatagaaa tttattaaaa attgctgact |
| 67981 | gttaaataat ttgtgtgatt acaacattta aagcaagatt tgaaaaattt ataagaaaaa |
| 68041 | ttcagggagt taatccactc tctttcctat gctgctagac cctattccag cggtctccat |
| 68101 | aaaaaaaatt cagagaaaca gaaagcagta acagtgatca gattgcatac aaactttctg |
| 68161 | cacacacata tatttgttat aacttatgta accgttgatt gatttggctt tttcctgctg |
| 68221 | gactatgagc tcctctgagg aatagattta ttttttttct gcatatcagt atcccgtgct |
| 68281 | tacccagtgt ctagtctgta acagccattc tataaatatt tatgggtgaa gaaaaatgtt |
| 68341 | gttgaatttt taaagtgaaa aaccaacatg gcttatcatc tctattttaa agattttaca |
| 68401 | aagggaatgg actgtgaaat ttccattaat aaaaataggt ctaatcttcc atgattgaac |
| 68461 | tatgatagaa ggatcttatg attgagtaag ctttttgtat tcaccttcat gttattttat |
| 68521 | cattttcaaa ataggaagtg aaggctcatt tcttgtgaaa aagaaatcta attcaattag |
| 68581 | tgtaggagaa ttttaccgag atgccgtatt acagcgttgc tcaccaaatt tgcaaagaca |
| 68641 | ttccaattcc ttggtaagtt aaattgtgca attgtgatta tgttgtgttt tgctgctgac |
| 68701 | attctcttga taactaaaat ttatgccaaa gctaggaaca attggtaggg atttccctga |
| 68761 | tgtatgaaaa ctataatttt gagattttta tatatgtaat agatatgaaa acatattaga |
| 68821 | tgtaaattat gctcaattca catttgtagt cttttgagta tgcagggtat gaattttttg |
| 68881 | gggcacatat atatatatat atatacttac agtacacttc aagatggttt tcttctttct |
| 68941 | tttcagaact ccatgtctga aaagagccca ggctagacct ctacctaatg gtgtgggttg |
| 69001 | gtcccatgaa cactttagct agaaatctga tagtgatttc taagaaacca gacagaagtc |
| 69061 | tgaaggacac tgaacaagat ggagtagcat aatataattc attgttcatc tatctatcta |
| 69121 | tcatctatct atctatctat ctatctatct atctatctat ctatctatca tctatctatc |
| 69181 | aatcatctat ctattttggt gtattgaaag tcatttaatt ttttagatac ctttattatt |
| 69241 | atttcaacct cttgtctgtt ttggaactat ggaaggacta tggcatattt gcatgaggag |
| 69301 | tctgataatt ctagttgagg aaattgggag ccaccttatt ctcaggttca ctttgaaaga |
| 69361 | cctgttctaa cctattctcc aattttgatt atagctgagt actaaaaata tgagggttgt |
| 69421 | tttgtgttaa ttctagatct taagatgggt gaaatgaatg actgtagttg aatcggttaa |
| 69481 | attagctgtc agtctttata tgctctttcg aatttatata taaatttagt tataaaaagt |
| 69541 | agtttggtta atgagaaatt atatggatat agctttttca ctcaaccttt ctgtttttca |
| 69601 | gtttccttat atttaaaaca aaggagaaag agtagatgct ttctaaggtc atttgagcac |
| 69661 | tgaactggag ttttctttta tcctcataat tgggttctta gtttttactt gcctattttt |
| 69721 | tcccataatt ataaatacca ttaaccctat taaaatttca tggttccttc cttataaaaa |
| 69781 | tgtcctcttc tccaataaat gacagcaatt ttattataaa ttatttttta ataggggccc |
| 69841 | atttttgatc atgaagattt actgaagcga aaaagaaaaa tattatcttc agatgattca |
| 69901 | ctcagtaagt atttggatgt aatcataagt aaatagatat tttgggcaga atgcagtgtt |
| 69961 | tggttgaatt tcctccaatt attcaaatat tcttggtgcc agtttcatct tacataatct |
| 70021 | tcatatatat ttacctaatg attccttcca taagctatag aaaaatgaaa catacattta |
| 70081 | aaaatttacc tttcttgaat attatagaac acattagtct ttttttttac aagttttctg |
| 70141 | aaatgtaaat aacacctcca ccaaggccac tcttcatcct ctcctccagt tttctttctt |
| 70201 | tctttttttt ttcacaccac ttacaccacc tgattaaatg ctttttattt actgttcatc |
| 70261 | tctgccactg gaatgtaaac tccatagttt ggttttctac tgaatcttca gtgccttgaa |
| 70321 | gaaagcctga ttgctaggag gtgctcacta aacttttact acatgaattt acattatttg |
| 70381 | cttatatttc cattgtttgt ttgtttttga caaaagggtc atcaaaactt caatcccata |
| 70441 | tgaggcattc agacagcatt tcttctctgg cttctgagag agaatatatt acatcactag |
| 70501 | acctttcagc aaatgaacta agagatattg atgccctaag ccagaaatgc tgtataagtg |
| 70561 | ttcatttgga gcatcttgaa aagctggagc ttcaccagaa tgcactcacg agctttccac |
| 70621 | aacagctatg tgaagtaaat ttaatttatc cttgtaactt tcaagacatt tgaagagctt |
| 70681 | ttgtatttat atctaatttg cataattaag tcgtttaaaa gaacattcta cttttgtgtc |
| 70741 | actgggtgat aagtcccccg tgcctctggt ttttgcacac atatcttagt ctgtgtgatg |
| 70801 | ttcaggagca tctttgaggg caggcaatgg aaacagatct gattagaaag gaattccagg |
| 70861 | ttctgtacgg agtacatgtt aaagtctgtc aagtgtatat tgattatact ttaatcattt |
| 70921 | aattcaagta agacaacttc aacaatttaa attagattag gtaaactaga attagacctg |
| 70981 | gtttggtagt actggctctg actcagctac caactgtgtg acaatatgaa catgtcactc |
| 71041 | cgcctgctct aatccttcat tttctcatct gtgaaataga gattcaacta aatgattact |
| 71101 | gaaagttttt ttttcagttt aaaataatgt gtaacttaaa gatttttttc tttttggtca |
| 71161 | aagttcctgt cttgtaagaa ttaaagtata acatagtttg ttgataggat agctctctga |
| 71221 | aaattgactt tgctcaccat ttgtatgtac tacagatcaa aatagttttg aaagccaaag |
| 71281 | aagatatcat aaaagttaaa attattttaa tgcaaatgtt taaattgtta aattctcaag |
| 71341 | gctgggcatg gtgactcatg cctataatcc cagcactttg ggaggctgag gcgggtggat |
| 71401 | ctttttgagt tcaggagttc aagaccagct taagcatcag caaaatcctg tctctaccaa |
| 71461 | aaatatgaaa aattagccag gtgtggtggg gtgcacctgt ggtcccagct acttgggatg |
| 71521 | ctgaggtggg aggattgcct gatcctggga ggcagaggtt gcagtgagcc aagatcgcac |
| 71581 | cactgtactc tagcctgggc aacagagtga gaccctgtct caaaaaaaaa aaagtcttgt |
| 71641 | tgcaaatgca tttccccctt tttaagccta aaaaattaat cataattttg agatgtttta |
| 71701 | aaggcaacat tacataaatt ttaagtatat ttaagggatg ttttttctct aaagttttta |
| 71761 | tatctggaga cagagagaag aaagaaaggt gacccactcc tccagccatg ccctaatgtc |
| 71821 | taaaatgtgt ctttctctct tctccacttc tttgccttgc taaatacttc aagccaccca |
| 71881 | gggctcaatt taactgtcac ttcatcactc tctgcttctc ttcctttttc cttctaccct |
| 71941 | ccagccaacc tacccaccta gcctccatgt ccataacacc tgatgctttc cacctaattc |
| 72001 | tcattcggtc atccaatttg atttaaaata cctttgcccc cactagactg tgaactcttt |
| 72061 | gaggacagga cttgtttcag gtttgtttct gtctattccc agtgcctagt gagatctgac |
| 72121 | atatggtaga agtttagtac ttactgaatt gatttgtgga ggaataaatg tctgaaactt |
| 72181 | ggtaatcctt caattaatat ttgttaaatg agcaagcaaa ataattttgg gatttagtct |
| 72241 | agttaaaaca aagagaattg gaagagactg tgacaaggtg agacatgccg gcattcaatg |
| 72301 | actggacaag ctcagaacct tctcttaggg aaatttcaaa atgacaccat tagatggcac |
| 72361 | tttgtttgtt tgtttgttat tgtcaaaggg tctcatctat gttcctttta taggaacatt |
| 72421 | tcctgattaa accttgggaa taattttaaa atctttactt cagaataagt taatgagggt |
| 72481 | ctgaaacaaa agcaggaatt ttgaaacaac ttctggggct aagagtggtt aataagcctc |
| 72541 | tataatgata tcaaactcta gagtttctcg tgtggataaa tatattgata aataaagaag |
| 72601 | accatagaga agtgattgat tttggtattt tagctctttg agagtattac gtacctaagt |
| 72661 | tttaaaaaat tgacataatg tgtaagtagg ggtttgctat tatcattata aaattagaaa |
| 72721 | ttgcttaaaa atagaaagta gaaatttgaa acaaaaagtt tcgtaaaaaa caggaggttc |
| 72781 | taaaatgaaa cacattataa gtaactattt ttatagttaa atcttaaata tatcaaaata |
| 72841 | tgtaaaattt ctgacagcat ttaaaacata ttcccaggat tatattgtac tttttgttaa |
| 72901 | atcattaatt caaatatttg ttgaggcgta ttctgcttcc attttgctct ttctggaaat |
| 72961 | aatttacaaa aaagctgaag gaagctttca actctatttt tgtgaacctg ctttttacaa |
| 73021 | tctacctgtt gtaattttcc tggttttacc catgctacaa gcagagacga ttgggccaat |
| 73081 | tagtatactg aaattctgtt gtggattgtg ttttcaactt tttgaaaatt cttgatggtt |
| 73141 | ctagttacca gaggtgtgta aggcagaaat attagctaga cttaagttcc tcagatggtt |
| 73201 | cactttagaa ttttaaacta ttgtcttttc agactctgaa gagtttgaca catttggact |
| 73261 | tgcacagtaa taaatttaca tcatttcctt cttatttgtt gaaaatgagt tgtattgcta |
| 73321 | atcttgatgt ctctcgaaat gacattggac cctcagtggt tttagatcct acagtgaaat |
| 73381 | gtccaactct gaaacagttt aacctgtcat ataaccagct gtcttttgta cctgagaacc |
| 73441 | tcactgatgt ggtagagaaa ctggagcagc tcattttaga agggtaagaa agagctcatt |
| 73501 | aaaaataaaa gggttgccta aatatgctga tgttaacaaa atatgctgac atttttatag |
| 73561 | caatgagttt taacaacatg gtgaaactcc atctctacta aaaatacaaa aattagccca |
| 73621 | gcgtggtggt gcgcacctta taatcccagc tactcagagg ctgaggcatg agaatcgctt |
| 73681 | gaacccagga ggcggaggtc gcagtgagcc gagatcgtgc cattgcactc cagcctgggt |
| 73741 | gacagaggcg agactctgtc tcaaagaaat atatatatat atatatataa tatatgtatt |
| 73801 | ataatatata atacatatat tatatatatt tattatatat aatacctata tattatatat |
| 73861 | atactatata taatacttat tatatatata ctatatataa tacttattat atatataata |
| 73921 | aaagaccgag gcaatgaata ttacaacttt tatcaactga cttacatttt tacaactaat |
| 73981 | ttttaaatta atgagtcctc tttgatgctg ttctttgaaa gcaaattgtt tttgatattt |
| 74041 | tttctttaaa gcatatgaat ttatgcaatt taatcattat cttgtctctt gtgactagaa |
| 74101 | ataaaatatc agggatatgc tcccccttga gactgaagga actgaagatt ttaaacctta |
| 74161 | gtaagaacca catttcatcc ctatcagaga actttcttga ggcttgtcct aaagtggaga |
| 74221 | gtttcagtgc cagaatgaat tttcttggta agtgttctgt gtgggtctcc tccttaccag |
| 74281 | gccctctaag ttgtacaaga tgagtcatat atggaccctt tagttgtgga tttaaaagtg |
| 74341 | gcatttcagt ttaaatatta tgctggattt aaaaaataaa attagcaggt tggcaataaa |
| 74401 | acaaaatgct ataaaactat gaaaagacat gaaagaaaca taaatgcata ttggtaagtg |
| 74461 | aaagaagcca atctgaaaag gctacatact attcgttccc aactataaga cattctggaa |
| 74521 | gagacaaaat tatgaggaca ttaaaaagat caatggttgt caggggttag gggagggaga |
| 74581 | gatgaataga taaaggacag aggattttta gggcagtgaa actgttttgt atactataga |
| 74641 | gggggatatg tgccatggta cacttgtcaa aacccataga atgtgcagta taaagaatga |
| 74701 | accctgatgt aaactatgga ctttgagtga taatggtgtg tcaaatattg gctcattgat |
| 74761 | tggaacaact gtgccaaact aatgcaaatt gttaataata ggggaaaatg tgtgtggggt |
| 74821 | ggtggtggtg gtgagggaag aagggattta ttgaaagtct ctggactgtg tgctcaattt |
| 74881 | tcctgtgtat ctaaagctgc cctaaaaata gtctataatt taaaaaaatt atcacatttt |
| 74941 | tattgtcaga ggttaaaatg atagttactt ggcctactgt gtagtaccct gtggttccct |
| 75001 | ttagtcttaa actaaacatg cacatggctg cctgagctgg gtaaggcatc ctgatactga |
| 75061 | gatattgttt ttcatactga agtttcttca gcaacttttt gtatgataaa tatgattact |
| 75121 | ctttgctgtt gttagaaata aaattaatca tttaatggtt ttcaaattag tgaagttaat |
| 75181 | gtatattcat tcagctcttg tgctttgcaa gacattataa taatgcaaat tattatcatc |
| 75241 | acttttatta aaagttgtag aatcccctgc cttctcttag catatgaaat aatagaggaa |
| 75301 | attatgttca tttgtatcct aaatgaacat tttaatttta aggaacaaaa tacttttatg |
| 75361 | acaataaaca ggaattcccc atattttatc ttccttcata gagaatactc acatcttgcc |
| 75421 | acccatgtgt ttattctata cctgactcag taaaataatt tttaattcta tttaatagca |
| 75481 | gaatatggct taactactta ttaatagtat tttaatttgt cagagctttc agaaactaat |
| 75541 | aatgatcaaa gccatcttaa tttggatagt attttcctcc ttttccgcgc cctcccttcc |
| 75601 | tcctaccctc ctgaatattg gccactttcc aaccatgatc ttgaaactgg ggatagagtt |
| 75661 | gttgactcac tcactagtaa gcaaagcaaa ctcggtctct gtccttaagg aacttgtgat |
| 75721 | taagttcaga gcacagacaa acattaagta atgatggcat aaataaatgt aattttatat |
| 75781 | tgtggtgcct tctgtggaaa agtttcagga tgctctgtgg tacctcaggg gaatctgatt |
| 75841 | cagactgtgg agtcaggaaa gcatttctgt gcctgtgaca tttaagttgg gtctggaaag |
| 75901 | aggagcatcc aaaaggacct tgaagcttgt gtatggtcag tgtgtcatct cagctgaatc |
| 75961 | tcacaatgat cctaatggtc atacctagga caggtatgac ctctgcccaa ttgaggaaat |
| 76021 | tgaagccgtg agattttttg tgtgatgctt acaggtgcat acaattaatt atcttgttgt |
| 76081 | gaaagtttga atggacccca gttcaaaatc tgtttgcttt gcactacaca atcttaatag |
| 76141 | tttgagaagt ggtcttacat ttgagaagat gccccatgga ggtgacatct gagcaggacc |
| 76201 | actaaccatt aaaaaataat cccatgtaaa tggtgaaacc ctgtctctac taaaaataca |
| 76261 | aaaattagct gggtgtggtg gcgtgtgact ataatcccag ctactcggga ggctgaggca |
| 76321 | gaagaatcgc ttgaaccagg gagtcagagg ttgcagtgag tggagatcag gccacagcac |
| 76381 | tccagcctgg cgacagagca agactccgtc aatcaatcaa tcaaacaatc aataaatccc |
| 76441 | atgtaaaata aagttttagt tctgtggcct tatgagtgtt ttccatacag catatgaaac |
| 76501 | tcaagactct gaagtcttaa gtggagaatc atttcgattc atttattttg cgaataggtg |
| 76561 | aggtataata gctatctttc tgcttctcag gaagacagct tctaggagtg tcctggaaca |
| 76621 | ttttgaccct tgaagattgt ctagataaag aataacccat atttttaacc ttgaaaatgc |
| 76681 | taataactaa ttcctcaatc cgttttccta gaaacggtag atttgtagat ctctcaagtg |
| 76741 | tcttagtgtt catctgatct agctacactc ttttacagat aagaaaagag actgtgtgat |
| 76801 | tttctgtttc ccaaattgtg tttcacagaa ttttatttct tggaggtacc tcatactgca |
| 76861 | tttccttctt aacaattcat aaagtaggcc aggcacggtg gctcatgcct gtaatcccag |
| 76921 | cactttggga ggccggggcg ggcagatcac aaggtcagga gatggagacc atcctggcta |
| 76981 | acatggtgaa accccatctc tactaaaaat acagaaaatt agctggacat gatggcacct |
| 77041 | gcctgtaatc ccagctactc aggaggctga ggcaggagaa tctcttgaac ctgggaggcg |
| 77101 | gagattgcat tgagctgaga tcacgctact gcactccagc ctgggcaaca gagcgagact |
| 77161 | ctgtctcaaa taacaataat aataataaaa taataaaaaa taaaaaaatc ataaaatata |
| 77221 | ccagagtatt gagaactcag atattttact ttattttact gaatgtttcc caatctctta |
| 77281 | agattatatg atcgaagaat acttttatta aataacaccc attattattc catagaacac |
| 77341 | tctttatgag ctagtaccat aaagaatttt tgtccttata atatactgta ttttagtaaa |
| 77401 | gtgttcatag ttgttttgct atagttattt aagagtaatt ctagttaatc tatatggtat |
| 77461 | ataataacat cacacataat ttgaaatgaa tattctagga atttttactt tgtacaaatg |
| 77521 | ttgggtagac aaatttatat ttaaatagct gtttagcttc caggttaaaa ggttctggta |
| 77581 | agtatagcat attattttat tgtttagtgg aagtattaag gctattacct tatttttaaa |
| 77641 | agtggatttt taaaaattgt cagtgtgaga atggaaatca aattaagtga cacactattg |
| 77701 | gtagctgttc ttatttttga atttaatgga aatctggtta aaatgattaa aatgtttatc |
| 77761 | tcattttttt tcttttagct gctatgcctt tcttgcctcc ttctatgaca atcctaaaat |
| 77821 | tatctcagaa caaattttcc tgtattccag aagcaatttt aaatcttcca cagtaagttt |
| 77881 | attgttattt taattttaaa agcacattag ctggaacaga acctttagaa acatgatttc |
| 77941 | gatttagtca tatagaggta attgatttct aaacctactc aacttgatgt ttttgtatgt |
| 78001 | atgaatgatt ttcactagat aaaagaccca actcattact taaaatggaa acttttatat |
| 78061 | ttatttagtg gatcattgtg taaaaacaac ttaagattgt ttaattaatt gctgtattag |
| 78121 | tataatgaaa tgagattata ctggctatca ctttaacttt taaaatttta attgtttgtg |
| 78181 | gaacttgata tgttgccaaa atacccttaa ctttcacatt atgcttaagt tatgtttgag |
| 78241 | tgaaattttg ggggaagatt aggcaagttt atgtagttcc aggtttttga gatattttgg |
| 78301 | ttaattcatg aaaccagaag cttcctgtta actttaaatt caggcattaa ttggatcttg |
| 78361 | agtgtgttgt aatcttaaat gctattctaa ttatatcata catgataaac caaattcata |
| 78421 | aaaatatatg tgtaaattta tcttttcctt tgttttcttg ctgtcagcta ttccttcaaa |
| 78481 | cactatggct ttttagaatt gacactaaaa tgctgcttgc atgatgctgc aatgagctct |
| 78541 | tctgtgagct tatatttagg caaataataa ttagaattta gccatagaga gtgttacaca |
| 78601 | aacctataat agctaaatta cgtctagctt tagaatgtgt ttaactgttc taactactct |
| 78661 | acagcggttc atctctttaa tcttcctaat aatgccctac gatagccact gttattatct |
| 78721 | ccacttcata agagatgaag taacttgccc agagtcatag ttcttaaaca ctggtggttc |
| 78781 | aacttcaggc tctcaaatca catgcatact catatgtcaa taatgctagt tttgacgtta |
| 78841 | cactttattc tcaccctggg gaaaattatt tgtgatgtta tttcatgtat tttggaaata |
| 78901 | ctcatttggt ttatgtcttt tctgtgtatt gttttagctt gcggtcttta gatatgagca |
| 78961 | gcaatgatat tcagtaccta ccaggtcccg cacactggaa atctttgaac ttaagggaac |
| 79021 | tcttatttag ccataatcag atcagcatct tggacttgag tgaaaaagca tatttatggt |
| 79081 | ctagagtaga gaaactgcat ctttctcaca ataaactgaa agaggtaaga cgattattgc |
| 79141 | cacttaaaaa atatacttta tgatttgcat cattacaaat tatcatttta agtgatattt |
| 79201 | agcttctaaa taccaatttc atgaaactag aagcttcctg ttaactataa attcctgtca |
| 79261 | actataaatc cagatttcca ttaaatttaa aaataagaac agctactaat gatgtgtcac |
| 79321 | ttaatttaat ttccattctc acaccgacaa tttaaaaaaa tctactttta aaaataaggt |
| 79381 | agtagcctta atacttccac taaacaataa aacaatatgc tatacttatc agaacctttt |
| 79441 | aatctgaaag ctaaacagct agatataaat ttgtctaccc aacatttgta caaagtaaaa |
| 79501 | attattagaa tattcatttc aaattatgtg tgatgttatc atataacata ttatagatta |
| 79561 | actcaaatta ttcttaactg ttacttaact atgacaaaac aacttagaac gctttgctaa |
| 79621 | tacacaagat agtataagga taaaaattct ttatagtact agctcataaa gagtgttcta |
| 79681 | tggaatagta gtggatgtca tttaataact ataaattcaa aataagcatt gtaaatatca |
| 79741 | ataccattca attttttttt gttttttaaa caagttgtaa gcctacccta tggtaaatgg |
| 79801 | atatggtaac acagcataat ttcctcaaaa aattactttt gtgatatact tttaaaggat |
| 79861 | tatatgaata tatacataat tatagatgaa tgtgatgctg tgtgtcattg tatcaccaaa |
| 79921 | tctctgtcca atctgttaac agactcttaa ataaaccatt tttctcaagt tgttactggc |
| 79981 | ctgtatactg tattacttgt ttttcagctt tccttggtac attattaaat ttctgcattc |
| 80041 | cgccaccatg ctatcaccct aatggatcaa ccttttttgt tttgctccat tctctctgtt |
| 80101 | gtaaatctga aattgataat ttgtttgtct cagaaaatat tatttttcaa gttctagcgt |
| 80161 | attgcctaca aaaaccaaaa gaattaagtg tctgacactg tgaggttcag caaaactgtg |
| 80221 | catatatttt gctacctgat tttttgccag caaatgagtg ttttctatta taaatatagt |
| 80281 | atatattgct taaaaatttg gaacagaaaa gaaattactc caattaggat gccacctaaa |
| 80341 | gtataagcat gtagctgtac tttgagaaca ctaaattgca tgcaggtttg tagtgactag |
| 80401 | gtcttcttgc ctttactgaa ggagcagaat gaagtcacag ataatggata accaaatcca |
| 80461 | ttttgtggta agaacttcct tactttcaat gtcttgaaga gatgaagtat gttaccaaag |
| 80521 | gagattgggt tttttaatat tacagatgag tgacatagat tgtttgggag taagttttta |
| 80581 | tatgtaagtt tttatgtttt taaacacata ctgacaactt atgacaaacc tttggaaagt |
| 80641 | tttaaaactc tgttgaaagg ttgtgcaagc tgctgatgga atctgtgagc ctttcttgtt |
| 80701 | tcttatcacg ttttttggca gagcacattt cttccttccc accaacaggt tttgcccttt |
| 80761 | tttttcccat taagattcct cctgagattg gctgtcttga aaatctgaca tctctggatg |
| 80821 | tcagttacaa cttggaacta agatcctttc ccaatgaaat ggggaaatta agcaaaatat |
| 80881 | gggatcttcc tttggatgaa ctgcatctta actttgattt taaacatata ggatgtaaag |
| 80941 | ccaaagacat cataaggtta gataattttt ttctatttgg ttttactaaa tttatttcag |
| 81001 | attttctact ctctgtgact ttgatggaca tatattgtta ctatttaggg aaaaataaat |
| 81061 | agtaatattt ggcattaata tgctgtgtgt catttgcctt tcatttaatg aatgtgtttc |
| 81121 | tgtggtgcca ctgtagagat ttctcattct tcttagccag actaatgttg agagcggctt |
| 81181 | ctcctccttc tgtttctttt cagtggagta gactctaaaa gaaaataagt attgctattt |
| 81241 | ggtctctggt taccaattac acaatctaaa gaaatacagc acagtataat aacttctcac |
| 81301 | actgtatttc atatagcaac tagttaacat atgcctctta catcttaaag cattatagct |
| 81361 | actgacatca tgtgaaatta ctaacttcta ttttgcccat taggatgagt aatctactca |
| 81421 | ccttgatcag ttttgaaagc accaaaactt ctcaagtatc actgtttctg gtctttacac |
| 81481 | tttaagcact ttaaatatct ttggtaatgg attttatcct cctttttgtt ccctttcagc |
| 81541 | acatcggtct tattactttc tcataaaatc ctttgctccc ttttccacag ttactgtatt |
| 81601 | aacgttgcag acctcagctc tgtcatcacc tctcaacttg actgtaatat ccaccaaggc |
| 81661 | agagaccatg gctgtgttca ctcactattc aaatcttggc acccaacaca gtgcctggca |
| 81721 | tacaattaat agttgtttaa ttaccagtga tttatactta ctcattctct tctgcctaaa |
| 81781 | atctcttaaa tttatattta acttcatctg tttttatgag gaaggatttt gttttctgaa |
| 81841 | ctcctgagct tgatttcatt ttaaaggagt ttgttatctt ttgtgctaat tgtggctacc |
| 81901 | cttcatccta cccaattatt tttctctctt gaaactggaa aagatggtca tataaaaatt |
| 81961 | ggttcagttc ttactaaaca tttagtagaa ctagctttca gtgtattata ctgtattatc |
| 82021 | taactaaata tttttaatat ttaatattta atttaatata taactaaata tttttaaaca |
| 82081 | tgtttaacat tttcagaaaa gacagaaaga cctagagcag attagaaatt gtaggcatca |
| 82141 | tttgcttttt gaagaaagac attttttcaa atagtggtgc attcttaaga aataaatcaa |
| 82201 | gaaaggtaat gttgcttttt ggtcatatca tcaggaatgt tggtcagatt cttattagtt |
| 82261 | acaggaatga attgatcact actctgatgt aaaattcact tatgatttag tctttttctc |
| 82321 | taatttgaaa ctgtggcaac attttaacat atttcaaaat atatctttct ctatccatta |
| 82381 | tatttttgat aacactttga ctctactatt agtttaaagg tggtttttta gctacctaaa |
| 82441 | cacttctatt tcattcaggt tttacattaa gatcattagg aatgaaagct aacatctgct |
| 82501 | gatagtataa tagtttatat ttatttatga tgttatgtga tctcactatc catatatact |
| 82561 | attatatgca tatgtgatat acatgaatat atagctatac atcatatata ccatatatga |
| 82621 | atatatacac acacatatat aatgtaacta atatgaccct attatcaagc tttaacagta |
| 82681 | tacatatatc tctaccttgt ttctatgtca tatggacttt gtgaaatttt gaactttata |
| 82741 | atttataggg tttttctttt cttttctttt cttttttttt ttttttttga gactgagttt |
| 82801 | cactcttgtc acccaggctg gagtgcagtg gcctgatctt ggctcactgc aacctctgcc |
| 82861 | tcttggcttc aggcgattct cctgcctcaa cctcccaagt agctggaatt acaggcacct |
| 82921 | gccactgttc ccggctactt tttggatttt taatagagac ggggtttcac tatattggcc |
| 82981 | aggctggtct caaactcctg acctcatgat ccgcccacct cggcctccca aaatgcaggg |
| 83041 | attacaggtg cgagccaccg cacctggcgt ataatttgta gggtttttca tactatttaa |
| 83101 | agacattaga atatgtatac atgtatgtat atgtgtgtat atatagaggt atatatatat |
| 83161 | tgcatatcgt attctaatta gtattgcaaa catattttgg ccttttgatt atttctggtg |
| 83221 | atagtgtaac atgttttctt tggtgatttt accaaacatt atcaactacc ctaaaatctc |
| 83281 | tagcaaaata tatgcattaa cagtactctg aaagacatgt acattattag ttatatgaga |
| 83341 | tatgcactct tctggatact atattttaga atagtgtgac atgtaaaaga actcacctaa |
| 83401 | atctcaagta tacttttaag cagtttatta ttttattttt atctttcaaa tactaggttt |
| 83461 | cttcaacagc gattaaaaaa ggctgtgcct tataaccgaa tgaaacttat gattgtggga |
| 83521 | aatactggga gtggtaaaac caccttattg cagcaattaa tgaaaaccaa gaaatcagat |
| 83581 | cttggaatgc aaagtgccac agttggcata gatgtgaaag actggcctat ccaaataaga |
| 83641 | gacaaaagaa agagagatct cgtcctaaat gtgtgggatt ttgcaggtat ttctttctat |
| 83701 | agaattttaa aattcacttt taccatttgt ttggaacagg gattcaaaaa ctgagctttc |
| 83761 | tgttctaata tccagaaacc tggtagactg tatggaatta ttccaaagcc cttcatttct |
| 83821 | cctaatttta cccttgcctc cagaatggag aagaacatgg agggatatgt taggaacaat |
| 83881 | ttggtgctag gtactttgat cggttgctga caaatatgct aaaagtggtc aatcctagta |
| 83941 | aaaacccaga atagttctct aaacatggtc tgttgttttt ctcttattag tatgctaaat |
| 84001 | aataaatagt attattctcc cagatttttt tttaaaaaag gattcttgcc tgtcgtttga |
| 84061 | aagattaaaa aaatttgtct ctaatcttta tttaggtcgt gaggaattct atagtactca |
| 84121 | tccccatttt atgacgcagc gagcattgta ccttgctgtc tatgacctca gcaagggaca |
| 84181 | ggctgaagtt gatgccatga agccttggct cttcaatata aaggtgattt gttctgatca |
| 84241 | tttgaaaata gaaaataatt catgtgtctg tgtgcgtgtg tgtgtgtgtg tgtaagttaa |
| 84301 | tttattttgg gcaaacaatt gcttcagtct ctttaaatac tttcttaaaa gaagcactaa |
| 84361 | aattttgaat tgggaaactt tccgagtaat gaagtcataa catgaaaatt gtatgttcca |
| 84421 | tgttggtgaa tgttattggt aacctgaaac tcttttatgc tgtaaaactt gaaaatatat |
| 84481 | atgttcaact gttttttaat tatattattt cttaaatgaa atctaaattt ttctaattta |
| 84541 | aaataagcta tattaaagaa aagcaatcta tatatatata tctcatcaac tttgtactca |
| 84601 | ggggccattt agtgtgaaat tcttcagatt gtatccttta agtggtccca gattattatg |
| 84661 | ctgttacatc tggaatctcc cttttgttgc ttttctatct tttcctttgt tgtcttgttg |
| 84721 | tcagctattc cttcaaacac tatggctttt tagaatggag actaaactgc tgcttgcatg |
| 84781 | atgctgcaat gaactcttct gtgcataaag tccttaaaaa gcttgtgtca ggacatttaa |
| 84841 | ccatgtaatt ggctgcatac atgcttgttt tgtaatttgg gtatttttta atgtttcttt |
| 84901 | tattaacttt tttacagcta gccaacgtga gcaaatagta cagtggcagt catatttgct |
| 84961 | tgagtggctt ttattctttc attgtagact ccaaattggt tgactttaaa acgaatttag |
| 85021 | aagattaaat tcacagataa ggaagagaaa atataaacta tatgacgtta atttgatata |
| 85081 | atttgtgggt ttatgaaatg cttattttat ttaggagtga ataactcatc ttaaggcatg |
| 85141 | aagatgggaa aggaaaacta taccactacc gttatatatg ccacctaaaa gggtgaagaa |
| 85201 | ttgggttaag aaaggccaaa aatgactttt taaaatgtcg taaggttaca tttttttctt |
| 85261 | aggtttaagg aaaaaaggac agttgttctt ttcttcttct gaagtctgct agtttctctt |
| 85321 | ttccattcaa gtgaatgtca cggaaagcaa atatcaacag gaatgtgagc aggcccagtt |
| 85381 | tgaaagcaaa cacaagaggg ttttgtgtct ttctctccag gctcgcgctt cttcttcccc |
| 85441 | tgtgattctc gttggcacac atttggatgt ttctgatgag aagcaacgca aagcctgcat |
| 85501 | gagtaaaatc accaaggaac tcctgaataa gcgagggttc cctgccatac gagattacca |
| 85561 | ctttgtgaat gccaccgagg aatctgatgc tttggcaaaa cttcggaaaa ccatcataaa |
| 85621 | cgagagcctt aatttcaagg taacatggta ggctggtaga gaaatgtaat ttattgattc |
| 85681 | tcaactgcct agaaatgtca gaaattttga gaagtgagca actcacttaa aattgtgggt |
| 85741 | tttctttcct tgttgctgtt agcattatta aagtcctttc cattttaaaa ttatttatgc |
| 85801 | cagacttcat ttctaattca tagaaatggg aacaaaaaat aattagagga acctgagaga |
| 85861 | aactaagaga ccgtttctgg gatactgaga aaatgtttct gagagagaat ctgagaaaat |
| 85921 | gtttttgatg ccttttctga ttcaacttct tatagtggtg attcaatcac aagggtaaag |
| 85981 | gtgaatactg aggtcttggg atcatctttc ttctattatt ctttaactgt tatttttcca |
| 86041 | tttcctcttt tcttttggaa ttcctgtttt atggacatct tgatcttttg tgccactcat |
| 86101 | tcatgaattt tgtcactgtg attcccattc caattttttt ccctccgtat tgtgaggcag |
| 86161 | ctgttttatt tagtcatgaa gaccactaac ttggttttca gcagtgtctc actaattact |
| 86221 | tagttcatac aaaatgggct ttttatttta ggaattatgt tttaaatgtt taaagttatc |
| 86281 | ttctcgtaag ccaaattttt ataaaatgta aataaatcag ttatcagaga gaacactttt |
| 86341 | ttttttaaat acttggcaga aaaaagaaat cttcactggg tactacaggg agtgtggtgt |
| 86401 | aaactgtact gaaaaatacc cttgatagtt ccatatgaca aacataatga tgaatttcac |
| 86461 | ttagtctgtc ttggcttagc tcaatagcac taatgatcaa gatactggct gataaataga |
| 86521 | gtcctatttg gcctgggcag tcccagcata attatgtaat agtgtcccac tatattctca |
| 86581 | aaagcattcc aatttggatg ataaattata tagtcacctt ggttataact ccatgctggc |
| 86641 | cagttagctt agttctgttc catttatata gattatgtgt gcttcactcc aaaacctaat |
| 86701 | gagccatttg taaaagtgat ggcttttgcg gtgcccaggg agagaatttg tatgtttgta |
| 86761 | tccttcaaca cacatttatt acagttatta aaaggtttta ttgatgatag atggtaatgt |
| 86821 | catgtaaaaa tgacatatta tttatttgta gactttccta ttctcttgtt ggacatgtaa |
| 86881 | ttagaaacta atatgactta aagaaaaaca aatacacaaa atttattcat ccaattaatc |
| 86941 | tcttaatcca ggtgtttttt ttttctgaga ctatacccat acttcaataa ctttgttgtt |
| 87001 | actgagaata ttttgagttt ccctttttgt cattgttgtc agagaatgta tcatatcttt |
| 87061 | aaaaagactt gttggaggat gagtttgttt tgaaaaggcc tgaatttagt tgatgcaaag |
| 87121 | tcacagataa gatggttcat taagctgtat taatactgct tttgtctaat agatatcatt |
| 87181 | accaataagt cagactagtt tttcttttgg cacttataaa tcacctttga agacaacttt |
| 87241 | ttacaaggaa ataaaacaaa tgctttgaga aataccagta ttattgaaag aaaagtatat |
| 87301 | attgctaatg gatgcagcat tctggcataa tggtttgaaa actcatttga ttgctttgta |
| 87361 | gaagaatgac tctttcagat gacccagggc ctgtgagcct gccagaactt gaaaattctt |
| 87421 | tcttccctga ggtgcttcaa cctgaattca aagagcagct tttaatctat tagagatcat |
| 87481 | tttttgtcct ctcatttatt tttcatattt gcctttgatc ttagctcttc tctaatcttt |
| 87541 | ttctgtctca accttattaa caggtgtctg tgcagacact tttaagtttt gttttttggc |
| 87601 | tcagcctgtc agttaactga taatcatgct gaaaggagaa gcaggacaaa acagagttca |
| 87661 | atgctgacaa tactcctttt aatcttgtcc agcccattag cagagcaggc atctctgtgg |
| 87721 | gccttgagac gtagtcccgt aaaactcatc ccgtttctac ttgatttgct ttctttgaga |
| 87781 | actcttgttt atttttatat ggaggtttcc tgccttggat taaaacataa acctcaatct |
| 87841 | gaagttcaat ttcatcttaa tttatgaacg actaagagag ggaacatgaa aagtggaggt |
| 87901 | tagtgaaatt atctctaatt ctctgggtta agagatacat gaaaacagtc tcttgagtaa |
| 87961 | ccatttgcag gtaaatatgg aagtaatggt tatggttgtc tctttaagtt tttagtcaca |
| 88021 | agtagaaaaa gaccaagtta atttttttct gtgtgtgctg aatttctatt tgtagtaagt |
| 88081 | gtaagaattt aagcagaaat tctgattcgt attttcagat aaaaagaata tgtaatttcc |
| 88141 | ataggtccag aaatagggag agtttgccat ctggtggttc ttaacggcac tctggatatt |
| 88201 | attaagagtt gcatttctat ttaaaattat attttaaaaa acgtttggaa gatactttta |
| 88261 | ttgtagaaac tatcctctta gggccattct ttaaaaaaat cttattttat atatttctca |
| 88321 | ttttgttgat agtgattaga ttctaagagc aacagaacaa tgatcatcct ctcctatcag |
| 88381 | aatcactgat gtttagatga tttctcattt tcccaagttc aaggttccat gaaaaacata |
| 88441 | gcttgagtgg gattttatgt ctctgcgttt cactgttgat atatatgtcc tcccaatata |
| 88501 | acattttaca aataaccaag cacaaaattt aatattttac cttgaatatt taaaatataa |
| 88561 | taatatccaa aagctcttgt aatttgtact gatatcttat actagcgtgt ctgtttcaca |
| 88621 | ttaagtttaa tgtcttagga tataaaaaat cttttttatg gttagtgatt tatcttgttt |
| 88681 | ttttttccat ggaatttctg gatagcgaga taaatatttc catactattt tatttgatat |
| 88741 | ttccaaattt gcctctgaat caacaatttt cctattttaa tttcattgta cttgttcctt |
| 88801 | acaacctaaa tagcttttta ttatattttg attttattta aaaatgtact tctgaataat |
| 88861 | atatctgttt ctgtaaaaac tgttagcact gaatttgcca accatttgac aaatacacaa |
| 88921 | ataaaataga tttttacggc ttgtcatttg taatttcata gatccgagat cagcttgttg |
| 88981 | ttggacagct gattccagac tgctatgtag aacttgaaaa aatcatttta tcggagcgta |
| 89041 | aaaatgtgcc aattgaattt cccgtaattg accggaaacg attattacaa ctagtgagag |
| 89101 | aaaatcagct gcagttagat gaaaatgagc ttcctcacgc agttcacttt ctaaatgaat |
| 89161 | caggtttgtg tttttcgttc cttattttca aagctcagct gtagtaactt ataaaagtgt |
| 89221 | ttctgaatct tttatagaat ttacattcaa agttgagaga atatccatac ggttctttaa |
| 89281 | taggccactg atttttttct ttttggaaga tcatcatgtg tgttcatgac aaatcatgta |
| 89341 | tcatgtcata agaaaacaaa tttagaaatc acctaggagt aaagcagtgg aaagagtccc |
| 89401 | tgagtgggag ttaaaatatt tgggttctag aacttgtctt tactattcag gagctgtgga |
| 89461 | accctgaata gtcaaatgac attcataatg tcaaatgagt ttagtgcatg tgaaagttat |
| 89521 | ttttatattg caaaggggaa ttattgttgg catggtctaa ctgggacgct tggagagtca |
| 89581 | atggctccct gagatgatgc agcttctgag tggaagatct agctctcttg catcaaatat |
| 89641 | tgatctcaaa gatgaaaatt ctcaaagcaa cttcagtgct aattgtgtac ttgatcatat |
| 89701 | taccttgcta gaaatgtgtg agttgtttga tagtactaga gtaagtgact gggaagctgc |
| 89761 | ttttgatccc tagattctgt tgtataaaaa atagcttccc gtggtttatg atctgttcct |
| 87821 | tttccccatc gttcttaagg tatgctgaga tatgctgtgt ttcttatctg tatttgaaaa |
| 89881 | taaaacatgt ctttgtagtg tgtattcagc aagcgaaaca gaaaattatg aatttctact |
| 89941 | tatgtgtgaa atatgctctg taatgcatgt cagtgtctca aatatgctta aatatgatca |
| 90001 | ttttatgtag tttaaaaata ctccattata atattggaac tttagaccat aggatgcaca |
| 90061 | gcttctagtc ccagctctgt cactagctat gctgaaattt cttcacctgc aaaatgagga |
| 90121 | agttggacta gattttttct aaagcccctt gatatttgtt ctagattcca tgtttcactg |
| 90181 | tttgatgact ttttactaca ggagtccttc ttcattttca agacccagca ctgcagttaa |
| 90241 | gtgacttgta ctttgtggaa cccaagtggc tttgtaaaat catggcacag gttggtgtct |
| 90301 | tttatttttg tggcacgggg gttatggtca aagcatagaa cagatggcgc ccagagcatt |
| 90361 | gagcatttta gaatttgggt ttagttaagg cagaaacttt tgtgaatttg gaaaactgtg |
| 90421 | gaacatttca catagaagac tacttgaaga gcttcatgga agaaggaaag atgtcttgag |
| 90481 | ttcacttcca tgacttggtt ttcaagccac atacagatgt ttgtatcact ctgccccatg |
| 90541 | ctgctttact agatcctgat gatgtcattg gtttggttac tgaattagtc aattgaatga |
| 90601 | tggctttgtg gaaatccttg gggtaaacac atataagaaa attaggttgc tgagcctgtg |
| 90661 | aaacctctat ctagataaca tggaggtgag ttttgactta agtgaaatga tctgagcttt |
| 90721 | aaatgcttac gattttgaaa actttggatg gccttggtta tagctatttt tttcttatat |
| 90781 | ttcacatgga aaatgatttt tttctccaaa tgataatcca ttaccaatga gtttaattag |
| 90841 | ttataataat ccatctctgt agctttgaca taaaagacca tttgagcaaa acatactacc |
| 90901 | tcagggcttt tcaaccccag catgatgaca ttttgggcca gataattctt tgttgcacat |
| 90961 | tgtaggatgt tagcagcatt tttggccttt atattcgaga cgtaagtagt atcctctagc |
| 91021 | tgtgacaagc aaaaatgtat ccagacattg ctaaatattg cttggagaat gtgaaaaatt |
| 91081 | accctagttg agaaacatta agctactgat ttgttgatga gtaaaattta tagttttgca |
| 91141 | tgtggctgcc cgagttccta aaattattat atatttttat gttagaaata tctcttccaa |
| 91201 | ttaaaccata aaggtaatta aattcactca ggcagccttg aataattgtt cctaaattcc |
| 91261 | atctaaggaa aaaaaggaag ctattgtgaa gagagaactc agttgaggct aaatcctgta |
| 91321 | ccatggaact caagagcata ttgaaacatt gcaatcagca attatttgca gtgtgtcagt |
| 91381 | tattactatt ttggtaggta tttttaaatt agattttcag ccttctgcac atatgtcatg |
| 91441 | gataatgtga ttttactcaa ttattaaatg ataatggaga cagtagtgtg acccagagca |
| 91501 | cttacttgag catcagcttg acctacgttt cagtctcttt aattacttat tagctctgtg |
| 91561 | aaatttctta atgcattaag cctttgttta cttacttttt aaataaggaa aataacaatt |
| 91621 | atcttctata ttgcctccct ggttcagtgt aagtgagggg taaatgttag ctaattttat |
| 91681 | attggatcta tttggcaatt taaagaatgt taatcaggaa attttaaaaa attcagaact |
| 91741 | ataaagaggt acttacgtag ttttggaaag tgtgtcatgt atggggacaa ataaaaaaga |
| 91801 | tgtgtaggta gctgcatcct gtacagcaaa ggaagtttta aatatatcca gcaattttgt |
| 91861 | tgtcctagct ggcgcacaat agttatcagg aggtaactca actccacata gtcaaggaaa |
| 91921 | agctaaagtt gctctctaaa gtggtgtgtt tccatgtcac tatggaacac ttgaagttgc |
| 91981 | acacatgtga acattaggat gggtatatct tatacagtag aataaggaag aggtttgcat |
| 92041 | cagaactccc cttttaaaaa aatgcagatt ttcactatga ctgcaataaa attcctgaag |
| 92101 | attctgtgga gtaattaagt tgaaactcca tgaaagttct tctcattagc atagttataa |
| 92161 | atatgataat ttaagtaaaa attaagttaa tttgagccac tcaaagttac ttttaaagac |
| 92221 | agatttaaaa tgtcaataaa atgataattt aaatttccga ttaacctaaa aaagaagtgc |
| 92281 | catcattttt atttatgcca ataaattgaa atataatgtc attttatcac taaggtttaa |
| 92341 | aggaaatgaa atctctaaat aatcaagtga aaccaagagc aacttgtctg acagctatta |
| 92401 | gcaaaaataa ataggagtat tcaccttcat gaatcaaggc aagggccgga ataatttcat |
| 92461 | ggtgcagaag ctctaatgag cccacccact ctatgcgccc cgagctgtta ggtcactaaa |
| 92521 | cttattaaaa aaaggtacca ttaaggcagg gagaagttta caagactcat ttaactgtat |
| 92581 | gataaaagag atatgaaaga gacctattca attaatcagg tggaacatta aaaagcttac |
| 92641 | atggcaattt aaccttgata aaaatacatg ggagaaatac aaaggaattt ggaaaattct |
| 92701 | ctttccttga ataaggcatc agttagctat tcaggttatg aggttgaagg aatgttagga |
| 92761 | gctcttttaa aggtgataaa gtcaagataa tgttgcagat tttattctta tgtaacaaac |
| 92821 | cccctcgaaa cttggaggct taaaatgtga acaatttatc atttctcgtt cttctgtggc |
| 92881 | ttgactgggc tcagctgcgt ggttctgctc cacatggtat tggcaagggt tattcacttg |
| 92941 | gcttcattca ttaaactgag ctggaaagtg caagaaaggt acatgcatgt ttttggagta |
| 93001 | ttggtgcttc tccatgtggc ctatcatatg gctaagttgg gcttcctcgt ggcacggtga |
| 93061 | tcacagaata attagacatc tttcatggtg gctggttacc aagagaaatg aagcagattt |
| 93121 | tttctgtcct cttaaaggct aggccaagga ctggcaaaaa tattaattct gctacattct |
| 93181 | agtaaccaga gcaaccacaa acctagctca gattaaaggg gaaggaaaag agactctata |
| 93241 | tgaatagcac ctatgtatag ggatggaaat gatgtgtcca tctttggaaa cttccactat |
| 93301 | aaatagtggt agcacgctat agatccacta ggaaaatcaa gcacaaactc tttaaaaaat |
| 93361 | aagtgtatct tagtaaaata gattagaata actagataat aatggctaac atacatgagg |
| 93421 | ttaatatgtg cttttcaaag attagctcat gtaattctca cagcaacctt tccaaatggt |
| 93481 | actttattag cccctatgat acagatgaag aaattgattg acagagaggt tgaataattt |
| 93541 | atccaacggt acacattcag gaagaggtag agttagaatt tcaaaccaag tagtttgact |
| 93601 | ccagggccta tgagtttata cattcatagg gctgatattc aaatgagaga agagaagtaa |
| 93661 | taaataaaca tataatatgt tgagtggtac agagtgctac aaagaaaata tgaagtgcag |
| 93721 | ttggagatga attgtcaaaa aaggtcttag cacttaaaaa acactaaaac agcaaacaat |
| 93781 | tctctttacc acctaaactg taagagcgat ctggaattgc tataaagtac acaacatggg |
| 93841 | agaagtctta aacaacagtt tttattattt ataggcccat tgcacactgt cattaaatac |
| 93901 | caatatgttc aatcaaccat gcattcattg attcaataaa tactgtacat acaaaataga |
| 93961 | aatacagaaa tgggtaagac aagtccttgg gcctaaggac tttataacct ggtatttcac |
| 94021 | tcaactacat gatagcataa ataatgtttg cttctgttta agtattcctt aaacattata |
| 94081 | gatctcccaa agaaaattaa atacaaacct ctttttaaag tgaatttgac aaagcaaaat |
| 94141 | aaattggaat atatagataa atatgctaaa atttgtcata tgtactttgc gtactttaca |
| 94201 | tgtgttattt cattctcagg gcaatctaag acagtcactt ttattatctc attttataga |
| 94261 | gaagaaagct gtgcagtaaa gaaatcaaat acctttccca aggttacaga gctagtagta |
| 94321 | gagcctggat ttgaatctgg gttctgactg atttttaact gccatgacaa ggatcaaagc |
| 94381 | tcaaagtgtg atctctgtgt tagaaacatc ggggttgctc tttaaaaaag ccgattctca |
| 94441 | ggcctcaacc cagacctact gacccagaca ctgcaagtag aatccatcaa aatgcagtag |
| 94501 | ttactttgag aatcatgaaa ctctgctaca cagtctgtct tcctattcat ggaagtcctc |
| 94561 | tcctagtata taaatgtgaa gtaatatttc tatttcaaac ctgtattgat aactgtctgg |
| 94621 | aagataattt tcctgggaat atattattga tgagactgca aaacagatgt gaggtattgg |
| 94681 | attagtcttt ccattgtagc tagggaaata ctgatgttca ttgtttcagt gaagttcaat |
| 94741 | gatttcctat ccgaattaac tcccttaatt taacaatttt tttttttttt ttgagagtga |
| 94801 | atgcccctct gggcttctag gccacatggt tgctagagaa attaggtact gtgttgcact |
| 94861 | tgaaaacact aaaatctttc tgactacttt cactgagcaa agagacataa aatgctttaa |
| 94921 | atttgcaaca tttcagaaaa taaattttag tgattattta tgactcgaat ctttcagatt |
| 94981 | ttgacagtga aagtggaagg ttgtccaaaa caccctaagg gcattatttc gcgtagagat |
| 95041 | gtggaaaaat ttctttcaaa aaaaaggaaa tttccaaaga actacatgtc acagtatttt |
| 95101 | aagctcctag aaaaattcca gattgctttg ccaataggag aagaatattt gctggttcca |
| 95161 | agcaggtaaa gaaaacctta aaaaattaat tgctacatgg aaattcacta tctattcttt |
| 95221 | taattgtcaa actaactgta gtctataata gatgtattaa ataaataaat atattttgct |
| 95281 | tctagtgtaa acctcctact gacatgtatc atttattttg gaataaaaca ttgcatctga |
| 95341 | cactttaaca atatagtaaa tcacttactt tatgtgtata gttactagtt ggcttatcac |
| 95401 | tgttgaaatt atttaagaaa ggtaaatagt ggagattaat gtgtgtgtgt gtctgtgttt |
| 95461 | gtgtatgtgt gtgttcttaa acaacactga gagagtttat taagcaagtt ctgagaagat |
| 95521 | agtgagtttt caacagaatt ttaaaagcat ttatggcatc acaatggatg cctatgtttt |
| 95581 | agcctatact atggaaattt ttcctactgc tctaagcaac tgggaaattt ataaagtaat |
| 95641 | atgatgttga aatgtgcaaa ttacattgat tgatggatgc agccaatttt aaaaataaat |
| 95701 | atacactttt tttctaggac atgtattttt caggatttat ataagattac atttgtctat |
| 95761 | gcataactaa ttgtaataat ttatgtatta gtgcacaggg attaccgaaa atatttcatg |
| 95821 | catctacatc tgagcatgca tttgaattgg ttattgacca ctgaattttt ggtgtaggaa |
| 95881 | aaatatgtag tgaaacaatg ttacaaaaag attacaattg tttggaatga ttaccttcat |
| 95941 | tgactttaag cagtaaaatc atttgctcaa caaggttggg tgttttgtga ggctgtataa |
| 96001 | ccatagtgtc cttttgcctt tagtttgtct gaccacaggc ctgtgataga gcttccccat |
| 96061 | tgtgagaact ctgaaattat catccgacta tatgaaatgc cttattttcc aatgggattt |
| 96121 | tggtcaagat taatcaatcg attacttgag atttcacctt acatgctttc agggagaggt |
| 96181 | aagtatctaa tgaagactta ttagattttt agagactatt aatttagact tattaatttt |
| 96241 | tagagaaatt agggagatgg catatgaaaa gtaatatgcc attttctcag agtttacttg |
| 96301 | tttggaaggc agctgaagaa ttagaaaata agctcataaa accttggagt aggcaatcta |
| 96361 | aagacacaca agcacatata acctcatcta atttgtcagg aagaaaattc cttaggtgct |
| 96421 | cactcagatc ttgactgtga ttacattgta gggactgtaa ttatctcttt tctgttgcac |
| 96481 | agccactaag acatttacaa aaaaagagca aatccggtgt ttataatgct aactctttct |
| 96541 | tctaaaataa atagagacat tttggtactc caaagggaaa atatcatttt ggggattaaa |
| 96601 | attagcttta cacaggtgtt actggtttcc aaaataaacc ttaccttgat tggaattaat |
| 96661 | caacatatag gtagttacat tgcattaaaa agttcagaaa gttttgcgtt tagcatgatc |
| 96721 | aaaaacttct ttttaaaaat tatgaggatt tatttatgat tttctttctt catctgtcga |
| 96781 | gcatattaaa ctgcttaaca gcatcaacct gaaatggatc ttaatgtgca ggggatttaa |
| 96841 | ctctttttat tgtaaagttg tggataaaat atttaataga tatggatgag gactcatatc |
| 96901 | agtaacaacc caatacttta tttcaaaatg aatagatctg tattacaatc acttgtgttg |
| 96961 | tgtgcagtag attttttccc tttaacttag gaagcagtta ataattaatg gctccatttt |
| 97021 | ttacaacgag cacttcgccc aaacagaatg tattggcgac aaggcattta cttaaattgg |
| 97081 | tctcctgaag cttattgtct ggtaggatct gaagtcttag acaatcatcc agagagtttc |
| 97141 | ttaaaaatta cagttccttc ttgtagaaaa ggtaaggaaa tcaatttgaa tgttttcaat |
| 97201 | tgcaacacta aagaaattta aacttaaaaa aaaaaaaaac tttaccttaa agctttgcga |
| 97261 | cagtatgagg tttagacaag gtgttgagct ctgttttgaa tcatgtaggc tgtattcttt |
| 97321 | tgggccaagt tgtggaccac attgattctc tcatggaaga atggtttcct gggttgctgg |
| 97381 | agattgatat ttgtggtgaa ggagaaactc tgttgaagaa atgggcatta tatagtttta |
| 97441 | atgatggtga agaacatcaa aaaatcttac ttgatgactt gatgaagaaa gcagaggaag |
| 97501 | gtatgttttg atacaactta caaatgcttt taagtgatcc ttcaatactt atgaagtgac |
| 97561 | ttttaataaa tgtaaatatt cttatccata agggatgagt tgaaaaatag tatattcaat |
| 97621 | tatagggaca gttcagaaaa ctgaattata tttattacca ataaaatctt gtattctaga |
| 97681 | ttcagaaaat gttgatttga gggtttgaat gctggcttat tgagcaacat aacctcatct |
| 97741 | gtgaaaccgg aataccaacc acatctatct catagaactg ttataaagat tcaaatagac |
| 97801 | aatacatgga cctaatttac caacatgtct gccatataat aacagctgca gcttcatgaa |
| 97861 | tgtggcaaaa gcagagagta gataactttc tagtcagatg tctggtagtc tgcagcagtt |
| 97921 | cagaattcta caagtgaacg taggaataag tttttaaaat tccaagtaga tagatactaa |
| 97981 | gtgaatcttt aaaatgttct caaatttcct agagaaatat aggattggtt agaaagggag |
| 98041 | ggattagaaa ttatagaaaa tattccatta ttttttcaca tcaaaaccac aaatttatgt |
| 98101 | atctccttaa atgttgtttt tatttaaaaa atgttttatt acttctcagg agatctctta |
| 98161 | gtaaatccag atcaaccaag gctcaccatt ccaatatctc agattgcccc tgacttgatt |
| 98221 | ttggctgacc tgcctagaaa tattatgttg aataatgatg agttggaatt tgaacaagct |
| 98281 | ccagagtttc tcctaggtaa ttctttttgt taatttgaga ataaaaatta ggatgtaatt |
| 98341 | ttctccttat aatttagaaa atagatttca taattatatt gtcatagatt ttactgtctt |
| 98401 | cqtatatttg ttataatttt tgtatttgga atgatatatt ttaaaggaat ataatattac |
| 98461 | agatctggaa tttgttttgc acataatcat gtagactagg atcaagatga ggatgagatt |
| 98521 | atcatggaag cagaaatatt tatgaaatat atctttgtat ttgccttaat tgccagggat |
| 98581 | atgggaggca aataagacag ttttcaggtg agttaagtga agcagccata ttttataaaa |
| 98641 | tgacagaata ggtaaaggaa gcacacctca gtgtagccat agcaggggtt ttatgactca |
| 98701 | gtgtgacaat gctgaattct catagaaata ttcattaaaa gccttgaaat taaagtcaaa |
| 98761 | agtgttacat ggtgacatac tcaaatactt tttttttttt ttttgatatg ctgaacaatt |
| 98821 | tacatttctt ggttccgtga attcaatcag tgattttcag tagagtatga tggaaatcat |
| 98881 | tgaattcatg tagcatgttt aggtgctcat tgagaaaagg tgaagtcatg gtaaccatgt |
| 98941 | ttcaatattc tcatttgtat cttgacttcc tgcacatgga tttttgggcc taaaagatgt |
| 99001 | ttttaaaaca tgctcataca cttcagaaga tgaaaagtgt atgcattata actactttgg |
| 99061 | gaaagaaaca gtcaacatat gttactgtat gtcattctgt atattacatg tgtggtttct |
| 99121 | catgtctctc agaataaaag ctaatgtctt tacaagacct gcgatgctgt gatctgtctg |
| 99181 | gctcctcggt tatcattttt aaaaaaagat atactttgta caaatttttt taattgacaa |
| 99241 | gtaaaaattg tatatattta tggtgtacaa catgatgttt tgatatatgt atatgttgtg |
| 99301 | gaatggagaa gtttagctat ttaacatata cattatctca aatatttatg tggtgagaac |
| 99361 | tattaaaatc tactctcata gcaatttaca agtatacagt atgttattat taactgtagg |
| 99421 | ctgacatact caagttttaa acattcctga gagtcattgg gacaactatg aaatgcatta |
| 99481 | gattgattta atataaagca tttgaagaca attttgacct tactttgttt agtttttgtt |
| 99541 | gttgttgtgt gtatacattt aattttaatc aaattacccc agaaataatg cctaagatct |
| 99601 | gtcagtcagg acataatatt attagcaaaa agttgtccaa aatttgagac atgatattta |
| 99661 | aagctaaata aactccttta tacccctctt attggcattg attgggaagt ttaggttgaa |
| 99721 | tttaaatgct ttggaactca ggaagttaat gtattagtaa tagtgggtta acataaaatg |
| 99781 | ctgaattgtc cttgctgaat cctacatctt aaccccagac ttcaaggtat acaggaaagt |
| 99841 | accagacatg gtgcatcctt cctctgaaga aatcccaaac tgtcagacac agatccctaa |
| 99901 | aatatttctt tttcctgcat taaaatgtgt ttcagatgaa tggacacgtt ttgagtagtg |
| 99961 | tatgtggaaa cgtcatttac aaagtctgtt tagttggcca ggtgtagtag ctcactcctg |
| 100021 | taatcccagc actttgggag gccgaggtgg gtgtatcacg aggtcaggag ttgaagacca |
| 100081 | gcctgaccaa gatggtgaaa cctcatctct actaaaaata caaaaaaatt aactgggtgt |
| 100141 | ggtggtgggc atctgtaatc tcagctactc gggaggctga ggcagagaat tgcttgaacc |
| 100201 | tgggaggcgg aggttgcagt gagccgaggt tgtgccactg cactccagcc taggcgacag |
| 100261 | agcgtctcaa aacaaaacaa aacaaaaaac aaaaaagcaa agtctgttta gctacccata |
| 100321 | taggaaaatg tttgtgatta ctctcccttc tctagaccca tgtcccataa atccataaat |
| 100381 | cccatgttca tttacagaaa gcagtctaga taggagtttc tcagtctttg agctgttgcc |
| 100441 | attttggctt ggataactaa ctctttctta tcgagggtca tcctgtgcac tgcagaatgt |
| 100501 | ttggcagcat ctctgtctat ccactagatg tcagtagtat ctccccttcc ctcagatgtg |
| 100561 | acaatcaaaa atgtctccgg atgttgccaa agataagggg tggggttgaa taccagtgat |
| 100621 | ttaaacaaat taggtgtatc cttctaaaaa cattttacag gtagcgactc cagcatcttt |
| 100681 | atattagagt aatctggaga aggttatgcc tctctcaatt ttccctcttt ccatttttat |
| 100741 | ttgtagggca gcaatgcatt caggcttttg gtaactcttt ttcccaagat agcagtaact |
| 100801 | attatgcagt gagtaatacg acccacctta atagatatga atagacttgt tttgtgaata |
| 100861 | tattttaaaa tataaatgta tgggattctg ttcatgcgtc tgagaagcca cagggtacat |
| 100921 | ttcctctttg tggagctatt tatttttctg gagagccaag acaggtattt ccacttcagt |
| 100981 | ggtgtgattt gaggggttag gaaaatttcc ttgccttcaa ttttctttcc aacctagatg |
| 101041 | tcacaaatac ataatagtag tccttaactt tatttttgtt ttcagtcacc tgaaagacat |
| 101101 | gacaatccat actccatatt aatgcagcgg cgattctcaa atagagaagg gctttaaaaa |
| 101161 | attagaaatc tctgccgggc gcagtggctc atgcctgtaa tctcaacact ttgggaggcc |
| 101221 | gagatgggcg gatcatgagg tcaggagatc gagaccatcc tggctaacac ggtgaaaccc |
| 101281 | catctctact aaaaatacaa aaaattagcc aggcgtggtg gtgtgggcgg ctgtagtccc |
| 101341 | agctactcgg gaggctgagt caggaaaatg gcatgaacct gggaggcgga gcttgcagtg |
| 101401 | agccgagatc gcgccactgc actccagccc gggcgacaga gcgagactct gtctcaaaaa |
| 101461 | aaaaaagaaa aaaaaaaaga aaaaaaaaac aactagaagt ccctactcca acttgaaatt |
| 101521 | tggatgtatc tccctagagt atgtttcttc tctatgctgc attgcaattt ttctttgttg |
| 101581 | ttgatagttg tccagattga ggggaggcag aacaagatgc atctatatgt ttccatctct |
| 101641 | ccgaccgatt ctctcccttc cccctctact tgctttcttt ctcttttccc tcttctgttt |
| 101701 | acccgattct atttctgatt ccagtatgta acagttccct ctgaagctct ctcaatacca |
| 101761 | acaatcctaa ctaatggttt ttaaaagtca aatattaagt actggaggga tagaatgaga |
| 101821 | gaataccaag actgataaga tgcaaataat acttttaaca tatttacaat ctaatagaaa |
| 101881 | tacaagacat gctcaaataa gttaattatt ttaatatact ctctctgagc ataaaatata |
| 101941 | attatatatg ctcattatag acatataaaa aataaatagg tagaggcttt ccatagatgt |
| 102001 | gtaatttcac cacttgaaaa ttactatatt tccttataga ctgttttgtg tgtattcact |
| 102061 | tatatccatc aagtgactac atttcaaggc actatatgag aaccataaat attgtacaaa |
| 102121 | caggatttgc taaatgtcgg tggagagtaa cagtccacgg ggctgatcat ggtcagtttg |
| 102181 | tgaggcaggc ctccaaactc cttggggatt gagatgatgg agtagcagag ctcttcaagg |
| 102241 | gtatggaggc ctgaaggtac aaagcatgct caggaaattt tggctattgc ggtttgtcta |
| 102301 | gagcacttgt tctcaacctt acctgctcat tactaattct actaagtaca gaattaaaag |
| 102361 | aagaaaaaaa tctaatgacc atttcctcct gggactaatt agatcaaaat ctttgaaccc |
| 102421 | agacattagc gttttaaaaa gctcctcaga tgtactattc agccaggact ggggcaggga |
| 102481 | aagctactga actccagcct tgagaatgag aagtagaaca agaggagaac tttaaaagga |
| 102541 | tttaggggcc actatatgac tatggagctg aatttagatt tgatttagta ggcaacgcgg |
| 102601 | aataatttgt ttctgaacag gagagtgaca caatcaaagt ggaatgatag gaaaattaat |
| 102661 | tttgcaagag agagagaatg agttggaagt aaggaactca gaaggcctcc tgggactcag |
| 102721 | cagaaagctc tgaggccacc aaatgggtgt ggtggtagtg gaaatggaga agaagggaat |
| 102781 | gtaaatgagg ctacacagtg gactgccact gttagccgtg gggttagacc acagcaagag |
| 102841 | ttaaaataat tcttcaattt taactccaga agggcctcaa aaagactttt tgtcttgtta |
| 102901 | tcatcagcta tatggaaggt agaataaaaa ctagttagga gaaaaggtaa taaatgtggc |
| 102961 | ttttgatagg ctgtgattga gttggaaggg cataccagtg aaatcaccaa cacaaagttg |
| 103021 | gaagtgtagg aaagcactta ggaggtggct ataagtgaaa atgtgaaaat tctctacatt |
| 103081 | aaagggatag atgaagtcac agaagtggat gacataattg agcagggtat gtgtagaggg |
| 103141 | aagacgggaa ggttaaggac aaaatcttta catatatctt tcttggagta gaaggaagag |
| 103201 | gaaatgttaa aggagatttg attcaatgaa acaagtaggt caggtttcta ttcaaattta |
| 103261 | caacagatat aattacaaca gatataattt atttagtttt tttcgcttgg acagcttaat |
| 103321 | ttaagtgctt tgtattttct tttcaaaagg tgatggcagt tttggatcag tttaccgagc |
| 103381 | agcctatgaa ggagaagaag tggctgtgaa gatttttaat aaacatacat cactcaggct |
| 103441 | gttaagacaa gtaagaaatt caataatata attatattaa attgcacatt attaatctac |
| 103501 | tggaactctt attttgcata cagttgtgaa aatgcaaaat aatgaccaca tttctactta |
| 103561 | agtttaatta tgcaatccta gtttgtcttt tcgttgtgga gtagaaagtt ttgtgttatt |
| 103621 | tctcctgttg agaaacaaaa cactgtatct gagaatcctt ataatcgtga tacatagtgt |
| 103681 | gttgtaaaac tttttgtaag actcacttac actcctcttt ttactttaga accttgctgt |
| 103741 | tcaaaatgtg ctccatggac aagcagccag gcattaccta ggagattgtt agaaatgtag |
| 103801 | aaacttggga cttttcagtg ccatattatt gttcctgata ctccacagta gtcagactcc |
| 103861 | tagctgcctc cacctgcttc cagaccttga agcctagcaa gctcctgact tcgccttctg |
| 103921 | ttttcttcag agtatttatc ttttactttt ctggtctagg gagagaatga tttttatttt |
| 103981 | tattgaacat gacttctgtg tgttcagggt gaaagaagaa gtttaatgca tgatctcaca |
| 104041 | ttgctaattt gattgaaggt tagaaatctt aaactaaaac tctcactgat aagcttgcac |
| 104101 | ctctcttttc tggatttatc cactttaata agaactgcta ttgattactt gctacaaaga |
| 104161 | tggagaaagt tagcatgctt atcctatttc ctactccctg tccctgtcca cttcctaaaa |
| 104221 | cttaaaattg gttgcattaa ttttcctgat atagtaacaa ttataacttg gaatgatttt |
| 104281 | caaaactttt gtttttttag tataccaact ctagacagca tggactgact ccttgctatg |
| 104341 | tgagatgagg aaaattaacg ctattctttc tccttttccc atcaccttct caagttcttt |
| 104401 | aatttattct attattttta tgtagtgaaa gtttataaca tttatattct ggtctgtact |
| 104461 | cataattaaa ttgttcacat tttgtctata gtttggttct gagaacaaaa ccaataaatg |
| 104521 | ccatttatat atttttttat ttgtacagaa ccaaaatatt tctacttcta gataaagaaa |
| 104581 | tgcaaccttc tgtcactaac ttcttttact aatagaatag taacattcca aatatcaaag |
| 104641 | tcaaatggat tctctattgt tatgtattta tcatcaattt ataaaaataa aggcatattt |
| 104701 | taatttggtc acatttttac cctgatttaa aaaaaaattt gtttttagag atggagtctc |
| 104761 | attaggttga ccaggctggt ctggaactcc tggcctcaag tgatcctctc accttaacct |
| 104821 | tctgagtacc agtggtgatt tattttatgt agctttttga ggttttctga ttatatacat |
| 104881 | atatttttaa aaaacgtact tcaggaaaag atatatattt tcatcatgac ttcaagtgtt |
| 104941 | tctaagttct taatcataca gtttgtataa cagaatctac tttcttcttg aagacattcc |
| 105001 | tcattcagca catgacttac tgctctaaac aggagagatg gatttctagg ctgcttgtgc |
| 105061 | agtgattaat ctatgagtta gtttcctcgc cctctttgat tactctcaat atttcttgga |
| 105121 | ttccatccat tctcttggtt ggattgtcct tagtttttgt tgaagaatat cttcgagtaa |
| 105181 | tttttttaag aaaaggtgtt tgtgaggtaa atgttttcag tccttacatg ttaaaaatat |
| 105241 | cttagttttg ccctcccatg tggtggatat gtcatcacac tttatttttt aggaatctag |
| 105301 | gcttgaaaca attttcttca aaatttgaag aaaattccat tgatttttag tgcccactgt |
| 105361 | tgctaatgaa aagtctgcag tcagtcagat gtttgctcct atctaggata cctttaattt |
| 105421 | cattttgaaa actgaaaatt tgaccttttg aatttcattt gttttcagtg ttctgaacct |
| 105481 | ttacaagtat gtgtttgtgt gtaggttgtt tttcattcca tctaattcat tattttgtga |
| 105541 | aaaattgtct tctgtgtatt ctcttctatt atttattatt tcctccctaa catttattaa |
| 105601 | tcatttttat tgacaactac tatgtaccag gttaggtgat gggacatatg atatatatat |
| 105661 | agtagtaagc taaacccagt caaggctctg cttctctgga gcctatatct agttacttat |
| 105721 | gattcattat tgcttatcat tgctccaaga gtatatgtta gatgacaagc cttttgggtc |
| 105781 | tatcatccat gtttgagttc cctcttcaag ttttatctat aatttgtgtt acttacttga |
| 105841 | ctgtctctta caggtttcta atatttttta gaattgcatc atctattatt tagctttctg |
| 105901 | gtgaattttg ttttgataat catattttcc atttccagca attctttcca tcccctctgg |
| 105961 | ttgttccttt gtagccatgt ttttggataa aatgtccata ggtgtttctg ttcatgtcaa |
| 106021 | ttagaatttt tttttgtatt acttgcatta ttgctttttt ctctgaggtt atttgctctg |
| 106081 | tgggttcatc ttgatctttc tcttttatct tgtcagtttt ccaaattgag tagttttggg |
| 106141 | tgacttcgta tgaagtaagt actctattga ttgttaaaga aggactgtat tgattattaa |
| 106201 | aggtaactag aatgggcatt cttcacattc atgtaggttt gcttgttcaa gttaccactt |
| 106261 | tctgaacaag aaggttagac catagacttt taagggctgc atactgcaaa gggatactct |
| 106321 | gttctttagg ttacatgggc agggatcact gctgagacca tacctgccaa aggaaggcag |
| 106381 | gctttgctct ctagatgctg gacttgaaat tgtttcccct ctgcttagtg ctgcattatt |
| 106441 | ttttttgctt cttaatctgc tgcagagtat ctagatcagg gtgtccaatc ttttggcttc |
| 106501 | cctgggccgt attggaagaa gaattgcctt gggccatata taaaatacac taacactaat |
| 106561 | gatagctgat gagcttaaaa aaattacaaa aaaaaaatct cataatgttt taagaaagtt |
| 106621 | tatgaatttg ggttgggcca cattcaaagc catcctgggc cacatgcgac ctgtgggcca |
| 106681 | caagttggac aagcttgttc tagatacttc agactctgtt ctacatctct tcatagatca |
| 106741 | ataacttgca gcaatgagtt tatcagataa attatgttca cttttcatcc ataaaaaaag |
| 106801 | tcatgggagg tactcaccat aggattggtt taatccagtc actctggcca attttcttta |
| 106861 | aaattccttt aaccttggta tattggttta attccttcag aatggctttt cccattgtga |
| 106921 | taaactggtt ccaggcttca cgagtactta ttccaagtac agaaggaaag agggtacctc |
| 106981 | ttttgagtac ttgtatttca aaggaatcaa atggcctaca taataaccca ttaaataaat |
| 107041 | agatgtctct cagccccagt gagtcatctg tgcttttctc aacaagcact atgttcaggg |
| 107101 | caatgctttg tgctaattgg ctttaggcct ttgttaccaa actgatcact gtggccctga |
| 107161 | gggtgctgtt acttagaata ttcccacctt ttctgagagg ctgatgttgg ggtcacttcc |
| 107221 | ccctgaacta aagtccaggg gctgcatggg tgagggttga gtaactcagt actctaagga |
| 107281 | ggaaaggaag gggaatatac actgttagtt aacagtggtt attcctgtat tctctcttgt |
| 107341 | ttggattcta ctggggattt ctttcttttt ttgagtcttt attgacatta ggaatgagag |
| 107401 | atagaaacag ggtgagagag gaagtaaaat taaatgtgaa ttcttccatc ttataccaga |
| 107461 | actcaactgt atttttggaa tctatatatc cttacttttc ccttgattta ttacaaaaaa |
| 107521 | ttcttagtgg ctacactaag taaattttgt aacctttaaa aaatacatag ttataatatt |
| 107581 | ttaagtactc tgagtaatag aagatttcat gtgagtacaa aatatcctgg gggcatttta |
| 107641 | attattagta acaacagtca cactgtagtt ctagaaccaa attgaacatt ttatatatag |
| 107701 | aagcttatct gtgaatcaaa tctgacataa tctcttttaa tgtgtaggta atttcttata |
| 107761 | taaattgatt ttttggcctt gctgtcagca tgcatttcaa attttacacc atgtgtttgg |
| 107821 | ggagctatgg tggaaattag tacattaaaa atgtctacag agccaattag tgtataagca |
| 107881 | gggaggcaaa ctaagggaat ggtggaagaa attaaaaaaa aaggggtgtg cataatgtgc |
| 107941 | ttcttctctt aaaaaaaaaa aaagaaaaag acaacaacaa aaagaaagaa aaatgacatt |
| 108001 | tgacggtcat tctaccagta gtcatggggc agatctatat tatactgtac tattacgctg |
| 108061 | ttttttcttt gcaattagtg agttgctttt ccaggataga aaatttggat tagacctctg |
| 108121 | tctgtgccta tgaaaacaag cagagctaac ttaactcctt ctcatcagtt ctaaccaact |
| 108181 | gacatgggca ttaaaaaaaa gattttatct actaagcaaa tatgatcaca gtctgaagct |
| 108241 | ttgttcttgg aaaatcccct ctcagggtgt tcagcctttc ttcttcagct tgcagaattc |
| 108301 | tccatgtttc agtttcctga taaatcagtg ggcgccgcta ctccacatct ttgaagctgg |
| 108361 | ttgttaagaa gcagtgcttc tgcagcaatc acagtttaaa gcatgaatca atttaacatc |
| 108421 | acacaagcta tacatttcaa cagagttaca gtttcagagt aaagtgcaat atacagtata |
| 108481 | aagcgaatct ggaattcaag cccaacaatg tcataaaaga ggctgtgaag tctcacatga |
| 108541 | tgtgggccac agagagggtt gttgccattg gatcttagct caaatactac cacatcttcg |
| 108601 | gagtggcctg ccatgggcca cccttactaa ggcaatccct ctgtccaagc caaaatgata |
| 108661 | tctcggactt tttttgaatt gcagagagat tgttgcagcc agggattgcc ttagtaaggg |
| 108721 | tggcccatgg cgggcctctc tgaggatgtg gtaatatttg agataagatc tgaatggcaa |
| 108781 | gacttcagcc atgtgaaggg tggagggatt tctagggaaa ggggtcagca caggcaaaga |
| 108841 | ccctatgatg ggaagaagct gggcacagct tgggattgaa tgaatgccaa tgtggctgaa |
| 108901 | gggtggtgat tgaagaggag ggggagacga gaaggtctgg aaggcctggg gcatgatcag |
| 108961 | atcaggtgct gaggccgtgg aagtagatgg gattttcatc taagggaaat gggaagtcat |
| 109021 | tagagagttt tcaggagggg atgatatgta tttttttaaa attgagcatt atcctcggta |
| 109081 | aacttttgta gtcgttaaac cagagattat aagcaggttt tacctcatat gccagttgca |
| 109141 | gctgattagt agtggctata gagaatcctg ggctgagaag gatactgtgg ctaaccagaa |
| 109201 | tttagtagat gagtttgacg tggcctgtta gtatgactac actgtgtgca ctgtttctgc |
| 109261 | attaaatgtc tgataaaaac agagccaaag gaaaaataga acttaaaaat ttaattctga |
| 109321 | cagtacagtt gacccttgaa aaacataaag gttggggtgc tgaccccttg tgcagtcaca |
| 109381 | aatttgcata taacttttga cctccccaaa aaactgaacc actgatagcc cactgttgac |
| 109441 | tggaagcttt actgataaca taaacacttg attaacacat gttttatatg ttatctgtat |
| 109501 | tatatactcc attcttacca taaagcaagc tagagaaaag agtattttat ttaaaaaatc |
| 109561 | ataatgaaga gaaatatatt taatcttcgt taagtagaag tggatcatta taaaggtctt |
| 109621 | caccctcatc atcttcacat tgagtgggct gaggaggggg aggtagagga aaggttggtc |
| 109681 | ttgctgtctc aagagtggca gaggtggaag aaaatttatg tatatgtgga ctcatgcact |
| 109741 | tcaatcccat gttgtccata ggtcaactgt agtttcaaaa ccagcttttt attactgaaa |
| 109801 | atacgggaaa aaaaactcag agaagaaatg gaaagtttgc tatgatccag tcatacagag |
| 109861 | aaatccatgt tcagcctgtt gatgcacttt aaagaaggag atacgtgggt aaaacctgat |
| 109921 | gttgaattac tcttacatga ttttggactt ttgcaggagc ttgtggtgct ttgccacctc |
| 109981 | caccacccca gtttgatatc tttgctggca gctgggattc gtccccggat gttggtgatg |
| 110041 | gagttagcct ccaagggttc cttggatcgc ctgcttcagc aggacaaagc cagcctcact |
| 110101 | agaaccctac agcacaggat tgcactccac gtagctgatg gtttgaggta agtaggtcat |
| 110161 | gttgttttct attcagtgca tgacaagtgt gatccagact tgctctcagg ttctgagaac |
| 110221 | acttcccagt aacactgtgc cccagtaaca atttataaac aatttggatg aaaactacca |
| 110281 | tttccctgat caaattttgt aatttcagaa aataagagta tggaaaccat gcagaacctc |
| 110341 | atagcaagta gtaatagact ttgaacccac aagttctgct ctagaaccca tcatcttaac |
| 110401 | cctgtactga tctgccttct ataaaaatgt ataagttagg cttcacagta tcaaagtaag |
| 110461 | tgtcaattac atgattccaa tgaggaaaga tgagtccata cttctcaagg ggactagagt |
| 110521 | gattcatgtt ggattcttcg gcatgaccat ctcacatgtc tcagaggcac acctaaccct |
| 110581 | gcatccagag caagctttgg agagggagca cactggagtg gaaaggctgt ggtctttgaa |
| 110641 | gacaaaaggc ctgggattca tcactattcc acacatttag taactgtgat tttatatctc |
| 110701 | tgattcccat tttttaaata gtctgtgaac catgactaat atttaatgca taaaattatg |
| 110761 | atgacttctg taataattgg agacattcca gatgaaactc ttgatgtccc ctctgccatt |
| 110821 | gctccccaac cccagtcacc ctgttacacc tgagagtcac cttacattcc tttcttcctc |
| 110881 | tctcatttca cagctaatcc ttcagcaaat cttttcagct ctgccaccaa aatatatctt |
| 110941 | aatgcttcta acaatttctc tcactaacgt ctaaatctga gccagtatca tctctcattg |
| 111001 | cctactggtc ccctgcttct acctctgtct catgatagtc ccattcctca cccagcctct |
| 111061 | ggagtgattt ttctaacatg aaagttggat caggacttgt tcctgttatt acccctcccc |
| 111121 | tgccttattt cttgggtaca gtgctcagcc actcccatcc ctgaggttcc ttgcagatac |
| 111181 | cagaggcttt atatctgctg ttgatttcac tcaggaatgt ctgactccca gatgtgctct |
| 111241 | ctacttatta taaaggatta tctgaatctt tctgaatcct ttcatttagg actctcagca |
| 111301 | gagaggatgt ccgcaacgac cctttgtctc tccagcccct ataggactat tgctgcctag |
| 111361 | gattctttat gttttcattt tttaaaaact tatttattgt ctgtcttgcc atcagaatct |
| 111421 | aagtaccatg aaagaaggga cttttcgtct tgtttgccat tgtatctcta gctcctaaaa |
| 111481 | tagtaagcct tcagaattac tgtgttgaca gtaggggaag ggggagaaag gaggaaagaa |
| 111541 | ggaaaacagt gcctggggca tagaagccaa gcagtgtatg caactttcct tctcttcttt |
| 111601 | ctcttctgaa atgctatgaa tatgcctttt aggtagtatc cagaaatgtt ccttcctgaa |
| 111661 | agggtccaga aactactgaa aactgtacag attatgaaat gaaacagggt gcagggattt |
| 111721 | ggatttgagt tgatgtttct gcttttgaac accaggggga atcttgggtt acattaatct |
| 111781 | aggtaaagtg cagaatagtc tcctgtattt cagtgccctc tttccttcat ttaactaact |
| 111841 | ctaggttcta gtttttccct aattcttcca caaatcccca aagtgtttat ttataaagtg |
| 111901 | aagaattgct attttttaac actgttcgaa acaccttatc tctaaaatga cttattctag |
| 111961 | ttctctgaaa ccttacttta aataacaaat ccagcagttt ctgatgaagt aaatgaaatg |
| 112021 | tcagcatatt ttaaaataat ttgcctaatt tgttcttagc ataatgccag aaaagctttc |
| 112081 | tggattttgt atcacaaaag gctagtagat ttcagtagct atcaatcttc taccagcact |
| 112141 | aagtatattt taaaaactca gcattaaggt ttatttttcc aagtatgttt cagcacagga |
| 112201 | aataaaatca tgctcctttg gagtccctta aatgctggag ctgtttagag tgacatacaa |
| 112261 | gaactttctt cacgttacat gctctctctt cctccatctt gcttttaact gttagcttac |
| 112321 | ttctccaatt caatccactt cgtttgaact ctttatcata attctataaa acttatgaaa |
| 112381 | atacagtcaa ctgcattttc tgtatgtttc tgtgtttcaa tatcttcaaa atggaatgta |
| 112441 | ctgccttggt acatcaccca ctatgaatct gttatttctg ttatatccca cagttgccag |
| 112501 | gccaggatac ttgtcccatc caggccaaac accttccccc gaaagcaagt atgcatttgt |
| 112561 | ccaccaggtc cttgactcta ttttacatta tctttttagt caattcattt atttttatgc |
| 112621 | cactcctgct gtcttggttc agtatgtcca gggaattatc agaatttctt ttctaaaata |
| 112681 | aaaatctgtt tatgcttgca attccttgac agttctcaat tatctgcaaa gtgcatccaa |
| 112741 | acttcttggc atagcatcaa agatctttct gtatgcctct tgcttccctt tgcggcccct |
| 112801 | gccaccccac tgcccacact gcattctagc cgtgatgaca ggcttgaatt ttcagttatg |
| 112861 | ctcatgtctg tccatcattg tatttgttat tcctctcttt cgaccaagtt gtctgcctag |
| 112921 | agagctcatt ttccttaaga atttcttcac aaaccatctc tactatgaag ctcaagtgtg |
| 112981 | tcatgaagtg ttagcttctc caacttgtgt ttcttgcaga cactctgtgc aagacattga |
| 113041 | cttaggtgct aaagagggaa agctagatat tatattgttc ttgaggttga aagcttacag |
| 113101 | tctagtagga gagtcaactt tgctgtcttt acctcagtgt ttttctccct ctgtgcttcc |
| 113161 | ctagcacgtg gtacttacat atttctggaa tcttgattaa acacctgttt gaggactgtc |
| 113221 | tgagcacaat ccttctggat tgtgacaccc tcaagggagc agagatacaa agatggcttt |
| 113281 | gtatactaaa tgactggccc tcatagatac ctagtacata tttgtcaaat aaatgaatgc |
| 113341 | attctatttt tggaataatt ctattcagaa tcagataaag tttactttaa gctatgaaga |
| 113401 | aagaagtctc ttagcaactc ttacaataat cacaatcaaa gaatgactgt ttaacttaat |
| 113461 | ataaaccagt ttgttttaat aaaatatttg acaatagtca tggttacaca atgcataaat |
| 113521 | tatggctaaa ttattatcag gaaggaaaaa tctttactta ttatttcaaa agctattttg |
| 113581 | ctagtctatt aaaagctatt agaactgcac ttcttaagat taaattctat aattgaacat |
| 113641 | tttaactaac caagatatta tctctttgcc actgacatta tttcaaatta agcttaacta |
| 113701 | tttcttttta gcctttggaa agtattctga aagagtctgt gttctataaa tatacttaaa |
| 113761 | gaggcatgtc ttataaagga tttggatact attcaatgat gtatgacttg gctttagctt |
| 113821 | ttttattctt aatctctcag cttttctctt cagcagggga agagtaccta atggcctttc |
| 113881 | agtaatccct tggtaaattt ttctttcaag cccattactt actgtgaagg tcaacttcat |
| 113941 | tagtgtattt atcttatttt tttcagccca aaataggtat attgaaatga atgggcctaa |
| 114001 | tgtcaaatgt cccgactaca tcctggaaga gagagaatct tcagctgtat tagttgatgc |
| 114061 | agttaaataa tatgtactct ccaggccctc atacaattga aagttcaggg tatcgttgct |
| 114121 | gctctgcttc taatccttcc agaagtgatt ggtgctaggt gatggagtaa ctattaattg |
| 114181 | atataatgtg agccaaaacc aacagtcacg aataagcaaa ggatttaaat ttaactccat |
| 114241 | taagtcttgt gagaaattat tttcaacata ggttataaca tacctgtgac atcacatgaa |
| 114301 | atgctgtagt caatttgaca tcatggggca gagaagacag agttggaaat cagaatttta |
| 114361 | tagacatcta atgtgataat aacattagta gctgagatgc ggtaagctct ttgaccatgt |
| 114421 | ttccagaatg gataagacct ggttgagatg aaaactttac actgtttttt tatattaact |
| 114481 | atcttttact ctttgcctga aatgtccaac tctagttgct cgtgattgcg tgggtcagtc |
| 114541 | tccagaaggt tggactttaa tattacccgt catcttttcc aagacaaaat tgtattcatt |
| 114601 | ctaactctta gccccaaatt ttctttttta accttaatat ctaacatgat taggtttatg |
| 114661 | gtaaattata tactcaaaca gaagaagaga ctaatagcaa gcaaaagtct tatattttca |
| 114721 | tttgttttca tccaaaaagt agaaaatatt ttccaaacat tgggaaacat tttagtcaga |
| 114781 | aaaataaata tcaatgataa atagaataga gaaaaatttt aaagctgagc taaacctcta |
| 114841 | tgtggtttta ggaaaatcaa aactattaaa taaatggcaa gtacaacaaa atcccatcaa |
| 114901 | ttcttattta acatacttac attttgaaat agttaaaata ttcatatgat cattgagaga |
| 114961 | attcagaatt gcctttaagt aattgttcac atatacaaaa gaaaagtctc caaaaattgg |
| 115021 | gtctttgcct gagatagatt tgtcttaaaa ttgaaatcat tcacttatca gatttgaccc |
| 115081 | ttttttaaag cataactttg ctgtgtaata ttagacttat atgttttgat ttccttctac |
| 115141 | aatatctctt aactttaagg gacaaagtga gcacagaatt tttgatgctt gacatagtgg |
| 115201 | acatttatat ttaaggaaat taggacaaaa attattataa tgtaatcaca tttgaataag |
| 115261 | atttcctgtg cattttctgg cagatacctc cactcagcca tgattatata ccgagacctg |
| 115321 | aaaccccaca atgtgctgct tttcacactg tatcccaatg ctgccatcat tgcaaagatt |
| 115381 | gctgactacg gcattgctca gtactgctgt agaatgggga taaaaacatc agagggcaca |
| 115441 | ccaggtaggt gatcaggtct gtctcataat tctatcttca ggatggataa ccactgacct |
| 115501 | cagatgtgag ttcagaagag tcaaaaggaa aacagagtct atcacattgt gaacagaggt |
| 115561 | ttattttgtg aaaaaatgca agcatcacat tgtgattttt atcattgtat tttgtaggaa |
| 115621 | aaaaacaatt gatgtaattt ttcagggcaa aaactgaata aaaagaagag aatgtttgat |
| 115681 | atcaagttat atgttttaaa gttagatttg tagattcttt agatactcta gaggtcataa |
| 115741 | aaagtaacag caaaaacttt agtgtaggta ttgttggcac ttgtgaggca aatcaaattc |
| 115801 | aggtccacaa attctttttc ataattctga aacccaaaga actctgaaaa tcccaagatt |
| 115861 | ttttaaaaaa tgactaattt ggtgtcaaaa cctaagcaag ctgacttgtt gcttattaca |
| 115921 | atctttattt ctcatgctca gtgtgaatat gcatacattt tgctgcagaa atatatacat |
| 115981 | gtttgagtac agggggctgg ccgtgaccct actgagggtt tctgtacaca tcactgtcta |
| 116041 | ccctgtggaa tcttacctcc ctttcttagt tcccaatcct gaaaagcagt tatggggcca |
| 116101 | gtgctctgta cagacatgtt gtctcagaca tcagtttgag caggaagtaa atcatttagg |
| 116161 | ggttggcatt tgtttggagt gtggggaaca ctctatcttt agggaaactt tatatagtta |
| 116221 | gttatttgta agtaaaatta caggtggcta tacatcatct tgctgattgc aactcaatta |
| 116281 | aatcaccgtg cctggcacag aagaaaatat gctacaggat atctcactag ggaaaaggtt |
| 116341 | ctagttcgtt tcctgcgcac tcaacttttg tacttagata agcaaatggc cccagattcc |
| 116401 | aatgcctggt tttatttttg ctccaaatac atatatactc ttttgttttg gatagttaca |
| 116461 | ttttagaagt agactgtgta ttctcataaa cacttcaaag tgtatgttct ggctgagagt |
| 116521 | gtctctgtgt tgttcaataa taataagact aattatcatt ttttgagtac ctgctgtgcg |
| 116581 | tcaggcccag tgccacgtat attagagaca agatctctta tcctcatgcc agggctggaa |
| 116641 | gttagctatt agtttctcat ttgccaaatg agaaaactga ggctcaggga gattatgtaa |
| 116701 | cttgcagaat atcactcagt aattggccaa gataagaatt cagtctaaat gagaaccaga |
| 116761 | tccagagata tttggcttta aattctatag tctctcctaa accatatgca actctaacat |
| 116821 | gaagaagctt atttaatctt cactattaaa aaagtcaaaa caaaacaaca gagccatgaa |
| 116881 | tagcaaatat tgtcaatgag aggtttggaa aaacagtctt aaaggatgaa attccataga |
| 116941 | cctgatatat ttccacctgg aaaaagtggg catgggacag tgattttctc ttgaaagatc |
| 117001 | tgctcatttt tgtcatggga catgaaggtg gactggacca ctcagtttct tctttctgca |
| 117061 | tctcccaacc cagtctttct gttcatgggg tgaaaatctg ttgttgaagc cttgtctgct |
| 117121 | taattggaca gtggatctct cgggtccctg tgggctgtgc gcttgtactt gagctctgct |
| 117181 | tcttcactct gtggtctagg ccagctagca gccagctgag ttcaccttgg ttcagactca |
| 117241 | tggcctttca ttttcagtat ctgacttcct ggttttgctg aaaacctgtc taaaatgtaa |
| 117301 | tatccatctg attcttcata ccaagccaca caattcttcc tgatcccttt taatctccaa |
| 117361 | tattgaatgg tggtaacata aatatggaga cagatcatgt cagaaaccca gggcctaatc |
| 117421 | ttttcttttc tgcctactct tctcacaggc tgcttagtac tttgtaagct tttttttttt |
| 117481 | ttctggctgt aacctagatt ttctctttat cattactcta tttattattg ttagagcact |
| 117541 | tctgattatc tcagccctaa actctgcctc caattttaaa taacaataac tcccactcct |
| 117601 | gctaatactg ctactactac taccatcacc aaactttttc ttccccaaag cagttctgtt |
| 117661 | tgggaaggaa acagttccct ctcatacaat ttcagttatc ttcttgtctt ttccgtttaa |
| 117721 | tgaatcttcc tgttaatgtt acatctttta acatggaaac ttctagagaa acaaaagacg |
| 117781 | atggatttgt taaacctttt gggtgtattt ttatactaac tcttactgca gcgtgtgcat |
| 117841 | tatgagtgta ggtccattac ggctgtatta ggagcagaac cttccagagc atgagcgatg |
| 117901 | tgctgggctt gtgcttagct ctatccatga gttaagtatc tcaatcctta ggaccctctg |
| 117961 | acatatgtgc tattattatt tctagtctat agatacagag actaaagttt agagaatata |
| 118021 | aaaaaacatt tacaaggtcc tatgggacaa aaactgtagg acaaaatgca aacccaagca |
| 118081 | gcctgagagc agagctcctg gtccagcact gtgatagctg gggacgcaga gacagaaaca |
| 118141 | atgcaattat tgacagggac catggtgctg tgtctgtcca cattttgaag ataattatgg |
| 118201 | tttggatatt ttcaccttta aataacttgg agagtttcaa cattaactca gtcagatgga |
| 118261 | tacatttata tcatatcctg ctgggagtga cagttaattc tgggtcctat ggcaattgca |
| 118321 | cttttgactg agatgaatgc tgactgatgg ctttaacatt taactaatgc gatagtattt |
| 118381 | aacacaccca tataaatact atagtcttcg ggtaaataaa atgttaccgg ctggacatac |
| 118441 | atgaatatct gatggagatt atggaacata ctctactcat acttctctga aagtaaaaaa |
| 118501 | taaaagatat gtttcagtac acaatgtgat atgtactcag acttaattca taaatttctc |
| 118561 | ttatccttca tccgtggatc ttttctttat ttacttattg cgtttgttaa aatgcaggct |
| 118621 | tctctgaaaa attattttta aaaatagttt ttagacaatg aatcatattt tctcaagtat |
| 118681 | tttaacattg taatcattat gataattatc caaggggaaa ttatacttat tttttattca |
| 118741 | tttattcatt catttggcaa caatacattg aacatttact aagcatcaaa ctggctctac |
| 118801 | cacttaatag tggcatgatg ttcatcaaga aattgttagt gcaatcaaga acactagaaa |
| 118861 | ttcattggat gaatttaaag aagcttttag aagggtatta tattataatt gaggcacttt |
| 118921 | atgaatatat aaataatatt atgttttcat gctagagatc atgccaatga agatatttac |
| 118981 | tttgaaaagg agaagattag aagtttaaaa gcatttccat attgaagtaa atattcattt |
| 119041 | ccatatcttc acagttatct ttctctgagt tctctgactc attgtgaaaa aaaatcccaa |
| 119101 | ccttcttcac agctctacca tcttcggatt gttgcctaga ggggtaaaaa ctattgtaaa |
| 119161 | aggatgtgtg cactggatga gaatttagaa ttagacgaaa tgacccctag agtcttttcc |
| 119221 | tgctttaaga gcctgtgatt ccaaattcta acagtacatt tatcaagaaa aaatatgctg |
| 119281 | aacatttaaa tagtttttga atagtaccta gatataatag atacctaata aatgtgctca |
| 119341 | atgaataata aataactggt taagatttaa ataagcctcc aaaatctctt ccacattcta |
| 119401 | agaagggaag cataaaggtt gttaatgaac tagtgactgt gtgggtagct cattattttt |
| 119461 | aagtactctt gactttgctg ttcattatct gtgtggcctt aggaaaatac acacatttct |
| 119521 | gaaaggatta tgtcgtttgt aaaatagaaa gtccttatct gtctaccaca gatgattctt |
| 119581 | atgcaaatca aatgaaatgt tcaataaggt gtctgtaaaa tagtagagag agatgaatta |
| 119641 | ggagctattg tgatttgttt acattatgtc acaggtgcac tttattaggg atatgtttta |
| 119701 | tcttaattac acaattcttt aacttagatt ttgagaatta tattatggtt atataggaaa |
| 119761 | atgcccttat tctaaggaaa tgtataatat atttaggtct gaaacattgt atctgtaaca |
| 119821 | atatagtatg taaattatgc taattcacat gataattata tgtaattata ttaatatatt |
| 119881 | actatgtata caatatattt acatgcatat atgtggggaa atgttatcag ttagtgtagt |
| 119941 | aggggttatc atactcaaat tcgatgtctc catccttcca actcttcatg cttttccagc |
| 120001 | atggtgagga ctgctgagct ccatcttttg ctggtagtct ctctgtcaaa tagaactgtt |
| 120061 | tccaaattca gtcatttgct ccttgaaggc tatgaattca tacttcgtta tatttttctg |
| 120121 | gctgcatatt taaattactt taacaatcat ataagttcat tgtaaaaatt ttggaaataa |
| 120181 | aaaggaagat aaaatgcaca gataatttta gcaaatgaaa taataattat attgggatgt |
| 120241 | atttcttcct agattttaat tatgtacatt cccatcaact ttttattttg aaaatgttta |
| 120301 | agcctaaaga acagttgaaa gagtagtaca ggctgggtgc agtggctcct gcctgtaatc |
| 120361 | ccaacacttt gggaggccga ggtgggtgga tcacttgagg tcaggagttc aagaccagcc |
| 120421 | tgaccaacat ggtgaaaccc tgtcactact aaaaatacaa aaatcagctg ggcacggtgg |
| 120481 | tgggcacctg taatcccagc tactcgggag gctgaggcag gagaatcgct tgaacccagg |
| 120541 | aggcagaggt tgcagtaagc caagatcaca cccctgcact ccagcctggg caagagagtg |
| 120601 | agactccatc tcaaaaaaaa aaaaaaaaaa aaaaagaaag aaagagtagt acaatataca |
| 120661 | ttcatactca tatacccgac gcataaattc attgattatt tactttttgc cctacttgtt |
| 120721 | ctttcttgct ctctttgcgt atgaatgaat cattgaaatt aagttgtaga catcatgcca |
| 120781 | tatcacctct gaacagtgtg tatatttctt tagaataagg atgtttactt acataatcat |
| 120841 | aataccatta tcacagctaa gaaaattaat tcagttgatt ttttccacat atttgataac |
| 120901 | tttctgtcta tccacgatta tgtcttacat attcttttaa tttatgtcat agcatatcat |
| 120961 | cttagaaagt gatccctaag ttactgcatg gtatacattg tttaaccatt tccctttgtg |
| 121021 | attggatgtc tttaggttga ttatattttt attattatca caaatgttga aatcactctt |
| 121081 | tttttctgaa gaatttaaaa gtaatttatc tgtcttatgg aataaaatat ttatttcccc |
| 121141 | ttaaaagaat ttcaggcatg aacccaagag agaaggcttt tttttttgtt ttagttgttg |
| 121201 | tttttatttt tattttttat tttttgggta gaaggagcag agagacaagt tcaggaaata |
| 121261 | atgagagtgt tagaattttg ttcaggttaa agtgagttgg agtgaagttt agaaatctcc |
| 121321 | tttctactca tctctcctgt ttttaaaaca ctgtcctgga aatagttaat attaggaacg |
| 121381 | agaaaaatgg tataggtttt cctagtacac tttatttctt aattatgaaa ttctacttaa |
| 121441 | taacttacca ttgaatgttt atccttatta tcattcaagg taattttatt gaagattgaa |
| 121501 | gatatttata ataaagattg aaggatttta ttgtcctgtg tggtcaacct tggggggtga |
| 121561 | gatgttatga gacaggacaa ttaattgact tgatcaaggt accttgttat aaaaataaca |
| 121621 | cagcctggtt tagaacatct cttcctgact ctcttatttg gcatatagcc taagtgtatg |
| 121681 | cctccttgga tgtatgagcc ctgatgttgg tcatatttat tattttatct gcttactttc |
| 121741 | agggtttcgt gcacctgaag ttgccagagg aaatgtcatt tataaccaac aggctgatgt |
| 121801 | ttattcattt ggtttactac tctatgacat tttgacaact ggaggtagaa tagtagaggg |
| 121861 | tttgaagttt ccaaatgagt ttgatgaatt agaaatacaa ggaaaattac ctggtaagtt |
| 121921 | ctgttttctc tacaatgaag attttttttc ttaatatcag cagcttcatt tttatttaat |
| 121981 | tgtagttgta tgcttaattc cttaaacaga tgatcatttt ttttgtttag tgcataaata |
| 122041 | ttcttaaatc ttgtgatata ttaataaaaa tcacctgaaa aaggtagcag ttttaggctt |
| 122101 | tttaaaaaat ccgcaattaa tattggtgta gttaatatta tatttagaaa catagagaag |
| 122161 | gaaattgctg ttagaactcc acatttggtg atttttaatt ttcataaaga attactgtgt |
| 122221 | actcattatc ctggaatgtt ttcgttttct tggagtgaaa taatttacat gcaggaatgg |
| 122281 | aagactgaat gatctataat aataattttt cataagaatc ggtaaatgtg tatttaatgt |
| 112341 | tatcaaagct catttggaat ggttgtctca tgctttcaag aaattagagg actttgtaat |
| 122401 | tcattcctta accattactt tagttctcac cacaaaataa cattttaagt ttatttagct |
| 122461 | ctttctcata ttttctgctt tccctttcat ttaaaaaata cttttgagtg tacacaatgt |
| 122521 | gccatgtaca ggaaatagag ctttatcttt tttgggtata acttcaagat catggcaaaa |
| 122581 | gaaaacttat tattaattgg ataaacctta gatataatct aggttatttc ccttatttta |
| 122641 | ctagttttct agtgaaaata ttcaggtctc tgctgggtac agtggcttac gcctgtaatc |
| 122701 | ccagcacttt gggaggccca ggcaggcaga tcacttgagg ccaggagctg gagaccagcc |
| 122761 | tggccaacat ggtgaaaccc tgtctctact aaaaatacaa aaagtagctg ggcatggtgg |
| 122821 | catgtgcgtg tagtcccagc taccaaggag gctgaggcac gagaatagct tgaacctggg |
| 122881 | aggttgcagt gagccgagat tgcgccactg cactccagcc taagcaacaa agtgagactc |
| 122941 | catcttaaaa aaaaaaattc agttctgtgt tctgcatcaa ccagaataag ctacgcctct |
| 123001 | tataaaaaac aaatgtgcac aaaccatctg tgaggacata aggattaaat gcttgcttac |
| 123061 | tttgagtatt aaaataaaaa gtagaagctt tattatatga gtaaaagtgt ttccaaagtc |
| 123121 | tatttgaaat gcaggtacag aatgaaaatc tgttatttta ttaaatcgtt atttgggtct |
| 123181 | ctttttattc cataaaaaaa aaatcttttc cacatctctt agtggagatc aagttaacaa |
| 123241 | aattagcttt aattttgtga caagtaaatt tacataaata taggattatg gagataatat |
| 123301 | ttttctttgc aatgtctgga ccttttataa acattgagag gaaatataac cattcttact |
| 123361 | tatttagtat gctagcatga tgttttttaa tgttttagat ccagttaaag aatatggttg |
| 123421 | tgccccatgg cctatggttg agaaattaat taaacagtgt ttgaaagaaa atcctcaaga |
| 123481 | aaggcctact tctgcccagg tattcttaaa gttttgttaa tattttgtac agaacatcat |
| 123541 | ttgcatatat gcatatatat ataatcttca ataatatata cttaaacaca taaacacaca |
| 123601 | gagacagaat taaaaatagt tataaggcaa acctcctata attttcacca tcccaggcac |
| 123661 | aaaaaaagga cattgccaaa acctcacatg ctcccatatg cctgtctcct cttctcttct |
| 123721 | ttgaaatgcc tgcccattat tttgcccatt tttctgttga ctcttttgcc tacaaatcaa |
| 123781 | atcqagaaaaa aattttata tataacatac taatcctttc ttggttttat atgttgcaat |
| 123841 | ttctttttcg agctggtggc ttgcttttta aatttcttgt agggtgcccg ttaatgaatg |
| 123901 | gaatttctta attttaatat atataaattt aacaattctt ctttttcctt cttttcctgg |
| 123961 | ttattcctat ttggtcctgt tgtagagccc atctttcttt tagagctaca aaaatattta |
| 124021 | cctgcaattt cttcaaacat tttaaaagtt tgcttttgac aagattttaa tccattggaa |
| 124081 | ttgacatttt tatctgtatc ccattccttt ttttaatgtg gaagactaat tttttcaggt |
| 124141 | tgatttacta aatggcttct ccccttttgt cccatagatc tgatgtgtcc attttgtaat |
| 124201 | ttattaaaga taatgtgcat atccacgtgt acactgtctt ccactgatca actcatttat |
| 124261 | ttttcctcca gtattaccct gtcttaattc ctgtagcttt ataatgatct cctctcccta |
| 124321 | tttatttttc tcatccagga atattttaac cagtcttagg cttagctttt ctatataaaa |
| 124381 | tttagaatca tgctgtcaaa tcttatgaaa aaccacattg tcgtttggat tggtcttgga |
| 124441 | ttgaatatgt tgacaatctg gagatgatat ccttatgata ttaagtcttt gtattctttt |
| 124501 | tcttatttat ttatttattt agaggcaggg tctcactctg tcacccaggc tggagtgctg |
| 124561 | gagtgcagtg gcaggatcac agctcaatgc agctttgacc tcctgggctc aagtgatcct |
| 124621 | gtcgcctcag tctccatcag ccaattgtgt gccaccagac ctggttaact tttcctttaa |
| 124681 | tttttttgta gagaaagggt ctcactctgt tgcccaggct ggtcttgaac tcctggcctc |
| 124741 | aagcaatcct cccgcctagg cctcccaaag tgcatgagca actgcacctg gccaagtctt |
| 124801 | cctattctgg aatggcatga attctctctg tttacttagg tcttctcaag cgtctttcaa |
| 124861 | taaagttttg taatgctaca tacaggtctt gcatatcttt tgctggattt tcttgctgca |
| 124921 | aattgcttta aatgcagttc atggcttgag atttgttttt taactgccca gttaacgtaa |
| 124981 | atctggtcta tgaattgtat tgacaacaag cttgtgagta gcaatttttc agcaaccttt |
| 125041 | tgctccctct ccccgctccc tgcacagtcc cagaacaact ttccttatat accaatgaag |
| 125101 | gtggaaatgt ggacaggttg atttctcctt cttgtttatg ctgaatatgc agtcctttgg |
| 125161 | ggtcccagct ttataggtac agtcccttta ctaagactgt ctatcttggg aaggccctag |
| 125221 | actccaactt ctctcccatg ggccccacaa agcatccaag agtatacatt tatattatct |
| 125281 | cactttgtct tggcagacaa atgtcttcag ggcaagtctg gctttagggg tccactgacc |
| 125341 | tctctggttt ctgtctctca ctgtgatttc ggcctgataa ttccttatgg tgtcagatct |
| 125401 | tcaatgtttt taatacattg tttttaaaaa aatcatttag cattttaaat tgtttttagt |
| 125461 | tggaagattt gcccaagtaa ccttgtccac catattatct gaaagagaac tcttgtcatg |
| 125521 | ttttacactg atacacattt taataaatgt gggttatctt tatgttgtga gctcttgatt |
| 125581 | tggtattata attaattgga aaagttttaa ctttaagtat tctgatcaaa atagtcaagt |
| 125641 | acaactataa tgactttatc aaatattaca taatttttct tctacttggt ttacttgttt |
| 125701 | tttaatttag gttaccatca atgttagtca catgaacttt tatatttatg tccacagtaa |
| 125761 | aaatttttca tagcttgttg tttttctttc ttgttttttc attttcaatt atacttcacc |
| 125821 | tatacttaac agaatactta acaaatatgt atatatatga caatattaaa aagcttagac |
| 125881 | atacttattt tatgtttaaa atataacata tactaggcaa gacaggaaaa ctcatcactt |
| 125941 | ttatgaaatg aggcacagac agagtaatgg gctttcttgg tgtctcctga gtggcgggca |
| 126001 | ggtggccatg tcacagctct aatcccagtt tcctgacttc tggttctgtt ttcttacggt |
| 126061 | gccttcacac tgtctctcca gatcaaaaac agaatctaga gatgacttct aaattttgtt |
| 126121 | accaaagact gaaattcctg ttcctttcaa ctactagaag ctcaactaaa ttgttggccc |
| 126181 | aagggttttt ctctcactgg cggtggctct gcaatataga attgcatgca gagtacctcc |
| 126241 | tgactccgct aaaatcctgt ttaattgacc cttgagattt tcttttcaag ttaaaaaaaa |
| 126301 | tactacacat accaaagagt atcaagcaca gtttaaaaat acatatttgt cttttcatgt |
| 126361 | aattttattt gtagtttaga ttactaatct tggtgatcta gttgggttcc agtttaacag |
| 126421 | ttttaggttt tgcttgacag agctataaca cttcagtcta tatttgattt ttcaagggaa |
| 126481 | atgagttaac tcgataagta ctgttttgtt atctttaaac tttctcaggt ctttgacatt |
| 126541 | ttgaattcag ctgaattagt ctgtctgacg agacgcattt tattacctaa aaacgtaatt |
| 126601 | gttgaatgca tggttgctac acatcacaac agcaggaatg caagcatttg gctgggctgt |
| 126661 | gggcacaccg acagaggaca gctctcattt cttgacttaa atactgaagg atacacttct |
| 126721 | gaggtaaatc caaatgctct ttaaatcttt cataatttaa agcatatacc atttggaaag |
| 126781 | ttacttagga ataaattaaa taagagccaa tgtaggatta ttattcaatt agccttctgt |
| 126841 | tagaacaaga gtattcaaga gcaaatgtgt tttgctttag aatcacagca tatgtcttag |
| 126901 | ctcagggtcc ctagaaacag agcctgagat gggttatctt gcacaagtga tttaaggaaa |
| 126961 | gagaatctta gagagaagta gaggaagcca gaaagggcag aggaaaaagc tcagcagaaa |
| 127021 | tgtggtttct gaagaggtcc agcctcagtc tctgcccaca aggagctctg gaatatgaat |
| 127081 | aggagcacag aattttccca ctgccaggca agggagccag tctttcattc tcacatattg |
| 127141 | ttcagtcatt ggctgcaatc tgctgggagg tgggggtagt gtaactttca aggcatttct |
| 127201 | gggcaagctg cctcctgtca tctgagggta ttctgtgata aatagcacat ctctgaacta |
| 127261 | tagtagccaa cactcagggt agctggggat ggcgtacctg atggataaag gagatctgga |
| 127321 | catggctcct aaaagtggat caatacattg tgttggcaaa tataagataa gtttctagac |
| 127381 | ttcaaagaca acctagtatt ttgactgctg cctgaagata aatattgtgc ctcaacatta |
| 127441 | gttctgaggt taaacaatct ttttttttta attgatcatt cttgggtgtt tctcgcagag |
| 127501 | ggggatttgg cagggtcata ggacaataat ggagggaagg tcagcagata aacaagtgaa |
| 127561 | caaaggtctc tggttttcct aggcagagga ccctgcggcc ttccgtagtg tttgtgtccc |
| 127621 | tgggtacttg agattaggga gtagtgatga ctcttaacga gcatgctgcc ttcaagcatc |
| 127681 | tgtttaacaa agcacatctt gcaccgccct taatccattt aaccctgagt ggacacagca |
| 127741 | catgtttcag agagcaccgg gttgggggca aggtcataga tcaacagcat cccaaggcag |
| 127801 | aagaattttt cttagtacag aacaaaatgg agtctcctat gtctacttct ttctacacag |
| 127861 | atacagcaac aatctgattt ctgtatcttt tccccacatt tccccctttt ctactcgaca |
| 127921 | aaaccgccaa cgtcatcatg gcctgttctc aatgagctgc tgggtacacc tcccagacgg |
| 127981 | ggtggcagcc gggcagaggg gctcctcact tcccagaagg ggcggccggg cagaggcgcc |
| 128041 | ccccacctcc cagacggggc ggcggccggg cgggggctgc cccccacctc ccggatgggg |
| 128101 | tggctgccca gcggagacgc tcctcacttc ccagacgggg cggctgctgg gcggaggggc |
| 128161 | tcctcacttc tccgacgggg cggctgctgg gcggaggggc tcctcacttc tcagacgggg |
| 128221 | cagctgccag gcaaaggggc tcctcacttc tcagacgggg cggctgccgg gcagagggac |
| 128281 | tcctcacttc tcagacaggg cggccaggca gagatgctcc tcacctccca gacagggttg |
| 128341 | cggccgggca taggctctcc tcacatccca gacggggcgg cagggcagag gcgctcccca |
| 128401 | catctcagac aatgggcggc cgggcagagc cgctcatcat ttcctagacg ggatggcggc |
| 128461 | cgggaagagg cgctcctcat ttcccagact gggcagccgg gcagaggggg ctcctcacat |
| 128521 | cccagacgat gggcggccag gcagagacgc tcctcacttc ccagacgggg tggcggccgg |
| 128581 | gcagaggctg caatctcggc actttgggag gccaaggcag gcggctggga ggtggaggtt |
| 128641 | gtaggtagcc aagatcacgc cactgcactc cagcctgggc aactttgagc actgagtgaa |
| 128701 | cgagactccg tctgcaatcc cggcacctcg ggaggccgag gctggcagat cactcgcggt |
| 128761 | taggagctgg agaccagccc ggccaacaca gcgaaacccc gtctccacca aaaaaatatg |
| 128821 | aaaaccagtc aggcgtggcg gcgcacgcct gcaatggcag gcactctgca ggctgaggca |
| 128881 | ggagaatcag gcagggaggt tgcagtgagc cgagatggca gcagtacagc ccagcttcgg |
| 128941 | ctcggcatca gagggagacg gtggaaagag agggagaggg agaccatggg gagagggaga |
| 129001 | cggagaggga gagggagagg aataatcttc ttatatggtt tgaaggaatg agaattcaca |
| 129061 | ctgaaaaata atttttaatt ttagtttcag atgtcatctt gataggcaaa acttgtctgc |
| 129121 | caattaactc atttattgct gaaaattaaa taaaattggc attgttttta aaagtaatgc |
| 129181 | aagaaagcaa aaagagttat gttgataaca gaatccttta ttctgtacaa gttctagttg |
| 129241 | cttaagctta aatcaaatcc tgctaagtat attttctttt cttaacagga agttgctgat |
| 129301 | agtagaatat tgtgcttagc cttggtgcat cttcctgttg aaaaggaaag ctggattgtg |
| 129361 | tctgggacac agtctggtac tctcctggtc atcaataccg aagatgggaa aaagagacat |
| 129421 | accctagaaa agatgactga ttctgtcact tgtttgtatt gcaattcctt ttccaagcaa |
| 129481 | aggtatggta gtgaatttga tcaatgggga aattacagat cttttaaacg actgaattgt |
| 129541 | gtgcataatt gttattgcat cagcaaagat tgttcatttt tagcctattt tcattggttt |
| 129601 | gcatatatta aagggaattg tggaaggtca cagagatatt tgttgttttt ctgaatacag |
| 129661 | atctagctga gacatttata aaataagtca accatttatt caggcctacc agccctgctc |
| 129721 | ctggtattac ctcaactgtg gctctatctc tttacttctc ctcagatcaa tgaatctttg |
| 129781 | tagggcctct tcaaggataa attctcattc attcattctt tgaaaaaaaa aaaatatata |
| 129841 | tatatatata tatgaaaccc attgtgtgcc aggcttaaac ataccagtta tctaactacc |
| 129901 | aaattaagaa aaaaattaaa taaatgaatt aataaattct taataggtga aaatgactta |
| 129961 | gctcttatca attgcagggt tcttgtccca aagaaatata tctacatagc aaaatttcag |
| 130021 | gtgtgagttg taggttggtg actgtaatat ttggggcagg atgatttcca ggaggcatta |
| 130081 | agattatacc ctatatattt ctctggttta agttagtatt ggaaaaaaag tactagaaaa |
| 130141 | atgtgaagcc tgttttttgt acctgaaata tcaactccac tggcagtttc ggagttgaaa |
| 130201 | ttatttgaat atggtcaaag aaaaatttca atggatggaa ttgggcaagg acgactttat |
| 130261 | tcaagcctat cacagcaggg gagagagatc agactgaact aaactccact gaaacaaaag |
| 130321 | gtgggagagt tttaagcgca ggggtgagct aatggaaacg tactggagca ccttgttgga |
| 130381 | aggaagtggg agcagttgtc aatgtgatta ggccatctgt gtttgctaat tgtcccttat |
| 130441 | tgaaggtagg ctcctactct cccacagaca ctggggaatt ccttccttcc ttccatccct |
| 130501 | ccctccctcc ctccctccct tccttccttc cttcttcttt tttttttttt tgaaggagtt |
| 130561 | ttgctcttgt tgcccaagct ggagtgcaat ggcatgatct tggctcactg caacttccac |
| 130621 | ctcctaagtt caagcgattc tctagcctca gcctcctgag tagctgggat tacaggcgtg |
| 130681 | caccaccaaa cctggctaat tttttacatt tttagtagaa acgggatttc accatgttag |
| 130741 | ccagactgat ctcaatctcc tgacctcagg tgatctgccc accgcagcct cccaaagtgc |
| 130801 | tgggattaca agtgtgagcc accacgccag gcctctgtct tgataattac atttcaaagg |
| 130861 | aatggctccc aggtccttgg aaaagacatt cttggggtat aaaactggga agagtctggg |
| 130921 | aaaaggggca gagaaagaat ttataattcc aagtcttcta aagtaaatac tctaagaaaa |
| 130981 | gggaggttag gaatttatag ttgagaagtc tatctaaagt ttaataaagt ggaggagaac |
| 131041 | attaaggcca ttttagtcaa catacatgtt ctttttgtaa caatttcaac atttttcctt |
| 131101 | ttagcaaaca aaaaaatttt tttttggttg gaaccgctga tggcaagtta gcaatttttg |
| 131161 | aagataagac tgttaaggta aatgttgaat gcattctaca tctaaattta ttttaagtct |
| 131221 | tttgttttat atatatctca cacccctctt atgggattat aaactccctg agagcaagaa |
| 131281 | tcataaatta tgctgtattt gtattgcttc ataaaatctt gaacacagta gatcctctga |
| 131341 | aaatacttgc tgattgactg tatattttat atgaatgaac taagaataaa atgataaatg |
| 131401 | acatctgatt gataatattg ggaatggaaa taattcaatt tgtacataac tgaggcagat |
| 131461 | aattccttat aaatatattg tggaaaaaaa acaaaaatat acttaagttt taaatatggc |
| 131521 | ttgccattaa ctttttctta agcattgaag aaatcattta attttctttt cttcagattc |
| 131581 | ctatttagtc attaaagcat tcatttctct atccatctat tcatctttgg ttccatctat |
| 131641 | tcactcaact tcctacccgt tcattctcct attgccaaaa agcttattat ctgatgagag |
| 131701 | acagggaagt agagtataac ccttaggtta tttcttttgt aatttttaca tgggaaaaag |
| 131761 | aatagattga atgtaacaat aatatttcga atatgaccta aattttttta tgtataatat |
| 131821 | ttgtacatat ttatggggta catgtgatat gttgttacat gcatagaatg tgtaatgatc |
| 131881 | aagtcagggt atttaggata tccatcacca tgagcattta tttctctgtg ctgagaacat |
| 131941 | ttcaagtctc ctagttattt tgaaatgttt ttaactgtag tcactttatt gtactattga |
| 132001 | acattagaac ttattcctcc tatctaactg tatgtttgta cccgttaacc agcctccctt |
| 132061 | catcctcccc ttctcccaca cacccatatc ctcccaagcc tctggtaact atcattctac |
| 132121 | tctctacctc catgagatca acttttttag ctcccacata tgagtgagta catgtgatat |
| 132181 | ttgtctttct gtgcttggct tatttcactt aacataatga cctccagttc catccatgtt |
| 132241 | gctgtatatg acatgatttc attccttttt atggtcaaat agtattccgt tatgtaaata |
| 132301 | cacacatttt ctttatgcat tcattcattc atgggtgctt aggttgattc cacttttttt |
| 132361 | tttagctatt gtgaatagtg ctgcgataaa catggggata taggaatccc tttgatatac |
| 132421 | tgattccctt tcctttagat tagtatcagt agtaacattg ttggattgta tggtagttct |
| 132481 | atttttaatt tttttgagaa atcaccattt tgttttccgt agtggctata gcaatttaca |
| 132541 | tacacaccaa tagcatatgg gcattcgttt ttttccgcat ccttgccagc atgttatttt |
| 132601 | ttgtcttttt tataatagcc attctaattg ggtgaagaag atttcattgt ggttttcatt |
| 132661 | tgcattttta ctgatgatta gttaatgttg agcatttttt ttcatatatc cattggccat |
| 132721 | tactatgtct tcttttgcaa atgtctattt agatcctttg ccaacttttt gttttgtttt |
| 132781 | aagacagggt cttgctttct tacccaggct ggctcacagt ggcatgatca tagcttgttg |
| 132841 | cagccttgac cttctgcact caagtgatcc tccaacatca gcttcacgag tagctgggac |
| 132901 | tacaggcgtg tgctaccata cctggctgtt tattttttgt agagatgcgg ctccactatg |
| 132961 | ttgtccagac tgatctcaaa ctcctgggct caagcaatcc tcctgcctca tcttctcaaa |
| 133021 | gtgctgggat tacaggcatg agccaccata cccagccctt tgcctacttt taaatggagt |
| 133081 | tctttttttt ttttcctgtt gaattgcttt ttcgagtttc ttgtgtattc tggatgaata |
| 133141 | gtttgcaaat atttcctcac atttaatgga tcctctctat attgttgata gtttcctttg |
| 133201 | ctgtgcagaa gctttttagt ttattatagt cccatttgtc taattttgtt tttgttgcct |
| 133261 | gtgcttttgg gatcttaacc ataaactctt tgtctagacc aatgttctga aatgtttccc |
| 133321 | ctgtttcctt ttaatagttt catagcttct ggtcttacat ttaagtcttt aatccatctt |
| 133381 | gagttgaatt ttgtaaatgg tgagagagtg gggcctactt tcatccttct gcatactgat |
| 133441 | atccagcttt tccagcacaa tttattgaag gtggtattct ttctcccatg tatgcttttg |
| 133501 | gtgcctttgt tgaaaattag ttggctttaa atatgtgggt ttatttctgg gtcctctaca |
| 133561 | ttggtctacc tacctgtgtt tttgccaata ctgtgctgtt ttggttactc tagccttgta |
| 133621 | atatattttc aagtcaggta gtgtgatgcc tccagctttg ttctttttgc tcaggattgt |
| 133681 | tttggctatt ttggctcttt ttcggttcca ctcaagtttt agaaattttt ttttctattt |
| 133741 | ctgtgagaaa tatcattgga gctttcatgg gaatttcatt gaatctgtag attgctttgg |
| 133801 | gtagtatggt cattttaaca atattaattc ttccagtctg tgagcatgaa tatctttcca |
| 133861 | tttgtttgtg tcctcttcaa tttctttcat gtttttcagg tttccttata gagatcgttc |
| 133921 | atcatctttg ttaaatttac tcctaggtat tttattaatt ttttgtagct acttcaaatg |
| 133981 | ggattgcttt cttgattttt cagctacttt gttgttgttg ttgtatagat atgctactga |
| 134041 | tttctgtatg ttgattttga atcctttact atactcattt atcagaacta agagtttttt |
| 134101 | ttaatggaat ctttaggttt ttgagtttta attttaatat ctctaatcat ttaagatgga |
| 134161 | aagtagtttt ttgaaagcac agattttatg gagttttgtt ctgtggatac tcaatttgct |
| 134221 | gagtgtgttt tctttttttt ttggcaaagc ttaaaggagc tgctcctttg aagatactaa |
| 134281 | atataggaaa tgtcagtact ccattgatgt gtttgagtga atccacaaat tcaacggaaa |
| 134341 | gaaatgtaat gtggggagga tgtggcacaa agattttctc cttttctaat gatttcacca |
| 134401 | ttcagaaact cattgagaca agaacaagcc aactgtaagt tattttttat ctgtacaagt |
| 134461 | aatttatcat tatacttttg ttttttcctt ataatcatta ataatactgt tgataattca |
| 134521 | taaggaagat cttttaaaat gcataattta ttttctatca taaaattaaa ctttcattat |
| 134581 | aaaaaatttt gaaaattcca gaaagcagaa gggtattttt aagaagtcac tcaacctaat |
| 134641 | cacttttagg gataaaatat gtaaactcat tgtaatctta gtagtattta tcaatctaaa |
| 134701 | ttttttaaca atttttatta tctgtgtttc aaattagaca tgaaattgga agacaatcaa |
| 134761 | ctttgtattt caccaaattc acggactata catatgcaat ttgggtaact tccattaagt |
| 134821 | attgattgta ggaaagatag acagcaagta ttcttgcttg tccaaagttg tttctagatt |
| 134881 | tgataattat acagatgtct actcacagcc aagttagtga taccagtttc aaacaagaat |
| 134941 | aaaataaaat attaatataa acctctttca agtttgcttt ttttcagtgg tattttaatc |
| 135001 | aaatctttgc agttggttct tatttatcac attcctcagt gataagcagt ataggattgt |
| 135061 | ggataagagc ataaatcata gttcagatat tgctttgcca tctattggtt ttgtgaactt |
| 135121 | gggcaataac ctttcacatc ttcagtcatc tcttacctga ggattgtaat attctctatc |
| 135181 | tcaaagagat atttggagga gtaaataaga atgttaatat atggatctta attaacataa |
| 135241 | tgaccagcac ctggtaagct gtcaataaac attagctatt attattatta ttagctttga |
| 135301 | gtcacaaatc cctaccttag ggaatatcct ggtttcccat tatccatcaa atttcccaag |
| 135361 | attggcactt gggagtaatc tttgactcct ttgttttttg ctccctttat ccacttgacc |
| 135421 | ctcctaattg tcatttgaat ctttgtactt ctcactgttc tcagtgctgg taccatgggc |
| 135481 | cagactgcca tccattatct gtggccacat taacaagagc atccagttct caccctctga |
| 135541 | ttatactctc ttcaacatat tttcagcatt gcagacagag ggacctttct aaaatgtaca |
| 135601 | tcaatctgat gcaattttcc tgcttaaaac ccttcagtgg tatcccattg tcctttatat |
| 135661 | gaagtccaaa ttccttaaga cagcctactg ggcccttcat aattcagtgc ttgcttacct |
| 135721 | gtctagattc atattttgca agctttttgc catctgtgct ttacccaaac tgaaattgac |
| 135781 | tcagatctct aagacaacct gttattacct ttcaccccca acctttgaat gtggtcttct |
| 135841 | ccttacctgg atgactatta tgccccctgc ctatttcagt ccaagcacca cttgctctga |
| 135901 | gtggcaggtt aggtgacttt gtgcttccat tgcatctaat gttttccttc acagtagcta |
| 135961 | taattgtatt tgttaaagta gtttctcaat accactaaat ctactggctt tcaacattgg |
| 136021 | ctgtgcattt agaaaccact aggtagctga aaataatatg atacctgggc cctacctcag |
| 136081 | accaattaaa tcagaccagt taagcctggg atggggatca gatttttttt ttcaggttct |
| 136141 | caagtgattc taaagtatat ttgaggttaa gatatactgc ggagtgcagt gtattataag |
| 136201 | ttcccatgaa tgaggatttt tatttctgtc tttacatatt tatttactag tatgtggtta |
| 136261 | acatttggat caactcattc tcattctgta atacccacat tttaaaaaat gaatgtaaaa |
| 136321 | atgtctttta ttatttttat ttttcaaatt taattttaga ttccaggaat acatgtgcag |
| 136381 | gtttgttaca aaagtatagt gcgtgatgct gaggtttgga atacaactga actcacaacc |
| 136441 | cagaaagtgg gcatagtgct tgataggtag tttttcatct ttgctcccct tcctgtaatt |
| 136501 | tccactttta acccaattat acaagcctga aaacctttaa aaagaaaggg cctccagttt |
| 136561 | atttttttat ttcatagcac attttatggg attatggata tcagttaact ctttaaagtt |
| 136621 | ccatataaga tttggagcat agatgcttta ctagagagca tccataaaga tcaagctctc |
| 136681 | aaagatgctc atctcccaaa agagattggg acctagatgc ataaacactt aaatataaat |
| 136741 | atttgctctc ttctgaacaa gtatctcctg tggccttggt ctctacccca caaaacagac |
| 136801 | atcacatcac taatcagggg ttgcctcatc atcagtaccc tcatcatcat cagtacacac |
| 136861 | cgactgagag gcatgttggg taatgaaaga tgactgcctt tgaagccgga agactccata |
| 136921 | ggacttttgg agttctaggt ctgcaacatg tttttagtcc taggtcggca agcatgatgt |
| 136981 | tagtcatagg accttgagca aattatttag catttgtaca tgtttctttc tactattaaa |
| 137041 | aacattgaga tttatcatat tttatgtttt tttattaagg atcaagcaag ataacacaca |
| 137101 | aaagtatttt ataaaatacg gaaattccat gcaaaacttt gtcctaattg gaactatttt |
| 137161 | ctattaaata cagcaaatat ccaagaagga attacctaaa gcgtagtggc tctctgacaa |
| 137221 | cacatcatat ttttacctcc ttttccagaa tagaaagaaa agggggagga aaaaaaatct |
| 137281 | tcctactcta ggatatacta atgattgtta aatctttatg gtattttcat gttatctatc |
| 137341 | tgatttaaat gcaattttga ctatttttac atattcctcg ttgttcattc atactgtagt |
| 137401 | gccttcttct attcccccac ttaacagact gcttgattat atcagaggtg cttatctgtg |
| 137461 | caactgttta ctggacgagg ggtatgtaga aaatacattg tcctcatcct tatgaaatta |
| 137521 | cactcataat ctagtgtgag ggatgcacta ataaagacaa ttttatattt aataaggtct |
| 137581 | ataatatgac agtgacacca gtggggaaaa gggactggtt ggtctatttt gataggtcag |
| 137641 | ggaagaaatc cagaggagcc gacatttaag ttcatcctca aaggccaagt aggagtttgc |
| 137701 | catgctgatg tggcccaagg tagccaatct gtttgtgaaa tatgtacaat gccagatgtc |
| 137761 | ttaggttgaa agggaaatat tttaaggtgt tcgtaatttt tctttatgtt taaaagggga |
| 137821 | aaatggcaaa tattttactt tctgtttatg tttggatgat gtggattttt gttttctata |
| 137881 | atttgactgg cttaactgca aagatatccc ttgctttaaa atttgaagac actgcaacta |
| 137941 | aattttattt cagcatttta tattttataa ctctaggtat aaaaggctaa cacttaattt |
| 138001 | tctgagcatt cqtgaaacaa agttttgcaa gaacattcaa aagttacaga tataatattt |
| 138061 | ccttcagaaa tttagatata gtacaaaatt ctacaaagag ccacatagaa ttgaaactaa |
| 138121 | aagtaagacc aaagtaaaca ttggacataa tctttatttt attatcacaa gaaattaata |
| 138181 | taaagtaacc aaaagtaagt aaagtaccaa agcatgttat atattcaatt cagaatggtt |
| 138241 | agggaagaat atgaaataat tgcaatagtc tagcttgttt agttttcaaa atagtgtttt |
| 138301 | tacattaaga actaatataa ggttgtatta cacgtagaaa ttttaagaag aaaacaaata |
| 138361 | gtgatgactt tctatttttt tttctctgta ggttttctta tgcagctttc agtgattcca |
| 138421 | acatcataac agtggtggta gacactgctc tctatattgc taagcaaaat agccctgttg |
| 138481 | tggaagtgtg ggataagaaa actgaaaaac tctgtggact aatagactgc gtgcactttt |
| 138541 | taaggtaaat tctgtggttt ttaattttat tcccaaaaga attatctttg cacttctagt |
| 138601 | gtcacagagg aaggattttt cttcctttct gcctctgaat agagaatttt tttaaaatgc |
| 138661 | agaaaaaaat ttgtaatgct tctcagcacc atcttttcag atcaagaaaa ttttgtcttc |
| 138721 | agaacataaa agaataggca cataatgtgc atagttttct catggtatta caaagaatgt |
| 138781 | tctcgaatga aaatactaca ttattgaaaa tgagcatatt ggagtctctg ctagctttga |
| 138841 | catagttctg tcacagtgtc aaatatacta tttataatta aattatgggc cccaggatta |
| 138901 | tctgctctaa agaaaaagag tcacaaaata atagacaaat atggggggaa atgcaatgga |
| 138961 | ctgaccgagg cgctaaggag tggggatcaa gaccccagaa tgagagcata gtgcttagtc |
| 139021 | tgatgcagcc tgtgagtgac aaatccatag caagcacatt ctttctgtgc tggtgctgag |
| 139081 | aaacaggacc attttcaagc ttatttgcta gccactttat atttttattt tgttttgatt |
| 139141 | ttaccatata gatctatgat actcttgaga acattttaga ttacacacta tatctgtaaa |
| 139201 | aggatacttc aaagtttcct gtcttagatt catctgacag tttttctatg gattgtgaga |
| 139261 | agggctcaca gtttatgttc agaagggcca acaggtctcc ttgataaagg gttccttact |
| 139321 | tcctgaagta ccaaaacgat taggattctt ttatttctgg acacttcatt tttgtcatga |
| 139381 | attagactat tcactggttc tgggaaaaaa ttcagtggtt tgtatcgata tcttttacat |
| 139441 | gtgaatgact ataattttat gttcctttgt aacattgaga cttcatgtaa aacttttgac |
| 139501 | tctaactttt ttttttcttt atcctgggca catgtatgct atttttactg aattagatag |
| 139561 | ctttggtatt tataaaaatt gtatccctct tattcataat ttcctgaaaa tgaagggcta |
| 139621 | ttgttatctt tgataattta tgcttcaagt aaagaagtgt ggctctttgg catctgtatt |
| 139681 | tagcaaaatt tgctttgtat aattttaatg atgcataatg gtggtggtgt catgttttaa |
| 139741 | taatttaaaa tgttgtttat gttatcatat gtaaatagca tttatctctt aattggtggt |
| 139801 | aaaattatta atgtatactt tatggttcta gggaggtaat ggtaaaagaa aacaaggaat |
| 139861 | caaaacacaa aatgtcttat tctgggagag tgaaaaccct ctgccttcag aagaacactg |
| 139921 | ctctttggat aggaactgga ggaggccata ttttactcct ggatctttca actcgtcgac |
| 139981 | ttatacgtgt aatttacaac ttttgtaatt cggtcagagt catgatgaca gcacagctag |
| 140041 | gcaagtttct ttcctttaga tatttttcat attctctaag tcttataaaa tatgccttta |
| 140101 | ttttacgttt acattttctc tgaactttcc agtgtcatat ggatggtctt ggagggtcac |
| 140161 | acagtgaaac ataagactgg tataaattgt gaatagggtc attacagaag tggagggagt |
| 140221 | aaatgctctc agtcccacaa gagaagcaga ttactgcagc tgaacactca gtttgggtct |
| 140281 | tacttgcttt tttccttttt acctaaggca aaaatgggaa atacatggta ttgaatatat |
| 140341 | tttacttttt gagcaaagaa aataaagaaa atgtttgttt taatcatagt ctagcctccc |
| 140401 | agcttgttaa agaatctcat ttggtttttc attctataac aaatcttttt tcttgcagca |
| 140461 | atacatgctg aactgcacaa cctacaaata ttgacaaatc atattttact caaactttgt |
| 140521 | ctttttttgc ttctattttt atatttaaat atgataaaat tgtgatagca cataaaatat |
| 140581 | attttctgca taaatatatt tgcgtcttcc tttgataata atttgtttta gaaaataaca |
| 140641 | ataatagcat atatacaaaa gtttacaaaa acgacactat ggggtttaat tctgaaaaaa |
| 140701 | actagaattt atgtaacttt agcaaataat gaatgttttg aacatggtga agaaaatata |
| 140761 | ttcattgcaa gtatatgtga aagaggaaca tgtgtttttc tagcaccttc acctattttt |
| 140821 | catttataga ctttagagtt gcacaggagt tacaattaga tgctcttaat gactgtaaac |
| 140881 | tattaagata catgtccaca caagcagagc agtaggtctc tcaataggtt gtctcagtag |
| 140941 | tcttattcta ccaaagttgt tgcattctct agttgaattg tatgtacttt gggacccaaa |
| 141001 | tagcttgctt ataactgaag ttatagtgga atgtctatgg gttatagttt gattttaaaa |
| 141061 | taaagatcaa ttggaggata gcctacaagg tgctgcatga gctggcttca ctgtacctct |
| 141121 | cctgcctcca tcatctacca cattcctacc agatcttgct tgtctagatg aacatccagt |
| 141181 | tcttctcaat tactatgcta tattttgcct cagggatttg cacatgctgt ttatttcttt |
| 141241 | gcttagttaa catagctttt tttcatgctt atttaactca tatgctttga aatgttagct |
| 141301 | cctgtgtcaa aacctctgag aagccaacac tgattaggtc aaagttcccg ctttgggttc |
| 141361 | ccataacagc tttcttgcat agttttgatc atggtcatat tttcttttca ttaatgctag |
| 141421 | tctcttcact agaatataaa ctccaagacg gttgggttag tgtgtttttg tttacccctt |
| 141481 | ttttcccagg atctagccta gggcctacat agaagacttt tgatgcaaat ttgttgaata |
| 141541 | aattagtgaa tgatttgaaa agaaaatatg atattttgac atagtatcag tatatccatc |
| 141601 | catctaagtg tccatctaaa tactcatatt tgtactaaat actcatattt gtaccttacc |
| 141661 | atatccaaag aacttttcac acacattacc tcgtttaaca ttgtaacttt gggaagggta |
| 141721 | atgtataaat actgagccta ttttatagaa aggttaagct gttttctcag actcacataa |
| 141781 | ttaagaattg cagcaaggag tggatcacag attttgttat tttttaaaaa aatgctggtc |
| 141841 | tttattcaat ataattgaag ggtcacctag aaaatagaat tgtgaattca gttccaaggt |
| 141901 | atttgtgtct taaactatga acaactttac ttttttttca ggccagttta atatatagtt |
| 141961 | ttaacagaaa acttacatat tttgtttttg taaaggaagc cttaaaaatg tcatgctggt |
| 142021 | attgggctac aaccggaaaa atactgaagg tacacaaaag cagaaaggta acatttagaa |
| 142081 | ggatactgtt ttccaaacag ggcaatgatg tgaatgatgg taacatatta tgtgtttcat |
| 142141 | aaatttgtag aaaatattac atatggtata atcaggaatt ttaattggta gtttatagtg |
| 142201 | taaagaactt agacataaat tttcaaaatt acaagtgata tgaagtgtta aatatttata |
| 142261 | ttttcagctg aagtagaggt gtcaatcact agctcaacct taaacgaaat gtgaatattt |
| 142321 | tttacaactt atctatatct acataatgtc taattttgaa cagtgtttga aaaagctttt |
| 142381 | atttctttta gaatatgaaa tgttaattta ttaaatgttg atactctatt tgaaatttaa |
| 142441 | tagtttctat aatgtattat aaaacttttc caagtatagt tttttataaa taataattta |
| 142501 | gtacattagt tatagctgtg tttatattta catttatcta agtcaactaa aaatacatga |
| 142561 | gccaaactga aataaaataa gaatgtttta tgatggatct ttgaaacatg atttcatttt |
| 142621 | tttctttttc tagagataca atcttgcttg accgtttggg acatcaatct tccacatgaa |
| 142681 | gtgcaaaatt tagaaaaaca cattgaagtg agaaaagaat tagctgaaaa aatgagacga |
| 142741 | acatctgttg agtaagagag aaataggaat tgtctttgga taggaaaatt attctctcct |
| 142801 | cttgtaaata tttattttaa aaatgttcac atggaaaggg tactcacatt ttttgaaata |
| 142861 | gctcgtgtgt atgaaggaat gttattattt ttaatttaaa tatatgtaaa aatacttacc |
| 142921 | agtaaatgtg tattttaaag aactatttaa aacacaatgt tatatttctt ataaatacca |
| 142981 | gttactttcg ttcattaatt aatgaaaata aatctgtgaa gtacctaatt taagtactca |
| 143041 | tactaaaatt tataaggccg ataatttttt gttttcttgt ctgtaatgga ggtaaacttt |
| 143101 | attttaaatt ctgtgcttaa gacaggacta ttgcttgtcg atttttctag aaatctgcac |
| 143161 | ggtataatga aaatattaag acagtttccc atgtaatgta ttccttctta gattgcatcg |
| 143221 | aaatgcacta tcatatatgc ttgtaaatat tcaaatgaat ttgcactaat aaagtccttt |
| 143281 | gttggtatgt gaattctctt tgttgctgtt gcaaacagtg catcttacac aacttcactc |
| 143341 | aattcaaaag aaaactccat taaaagtact aatgaaaaaa catgacatac tgtcaaagtc |
| 143401 | ctcatatcta ggaaagacac agaaactctc tttgtcacag aaactctctg tgtctttcct |
| 143461 | agacataata gagttgtttt tcaactctat gtttgaatgt ggataccctg aattttgtat |
| 143521 | aattagtgta aatacagtgt tcagtccttc aagtgatatt tttatttttt tattcatacc |
| 143581 | actagctact tgttttctaa tctgcttcat tctaatgctt atattcatct tttccctaaa |
| 143641 | tttgtgatgc tgcagatcct acatcattca gatagaaacc tttttttttt tcagaattat |
| 143701 | agaattccac agctcctacc aagaccatga ggataaatat ctaacacttt tcagttgctg |
| 143761 | aaggagaaag gagctttagt tatgatggat aaaaatatct gccaccctag gcttccaaat |
| 143821 | tatacttaaa ttgtttacat agcttaccac aataggagta tcagggccaa atacctatgt |
| 143881 | aataatttga ggtcatttct gctttaggaa aagtactttc ggtaaattct ttggccctga |
| 143941 | ccagtattca ttatttcaga taattccctg tgataggaca actagtacat ttaatattct |
| 144001 | cagaacttat ggcattttac tatgtgaaaa ctttaaattt atttatatta agggtaatca |
| 144061 | aattcttaaa gatgaaagat tttctgtatt ttaaaggaag ctatgcttta acttgttatg |
| 144121 | taattaacaa aaaaatcata tataatagag ctctttgttc cagtgttatc tctttcattg |
| 144181 | ttactttgta tttgcaattt tttttaccaa agacaaatta aaaaaatgaa taccatattt |
| 144241 | aaatggaata ataaaggttt tttaaaaact ttaaa |

For example, the nucleotide sequence corresponding to the mRNA of the human LRRK2 is depicted in SEQ ID NO: 10 (9239 bp), wherein the underscored bolded “ATG” denotes the beginning of the open reading frame. Sequence information related to LRRK2 is accessible in public databases by GenBank Accession number NM_198578.3 (nucleotide).

SEQ ID NO: 10:

| | |
|---|---|
| 1 | gcgctggctg cgggcggtga gctgagctcg cccccgggga gctgtggccg gcgcccctgc |
| 61 | cggttccctg agcagcggac gttcatgctg ggagggcggc gggttggaag caggtgccac |
| 121 | catggctagt ggcagctgtc aggggtgcga agaggacgag gaaactctga agaagttgat |
| 181 | agtcaggctg aacaatgtcc aggaaggaaa acagatagaa acgctggtcc aaatcctgga |
| 241 | ggatctgctg gtgttcacgt actccgagca cgcctccaag ttatttcaag gcaaaaatat |
| 301 | ccatgtgcct ctgttgatcg tcttggactc ctatatgaga gtcgcgagtg tgcagcaggt |
| 361 | gggttggtca cttctgtgca aattaataga agtctgtcca ggtacaatgc aaagcttaat |
| 421 | gggaccccag gatgttggaa atgattggga agtccttggt gttcaccaat tgattcttaa |
| 481 | aatcctaaca gttcataatg ccagtgtaaa cttgtcagtg attggactga agaccttaga |
| 541 | tctcctccta acttcaggta aaatcacctt gctgatattg gatgaagaaa gtgatatttt |
| 601 | catgttaatt tttgatgcca tgcactcatt tccagccaat gatgaagtcc agaaacttgg |
| 661 | atgcaaagct ttacatgtgc tgtttgagag agtctcagag gagcaactga ctgaatttgt |
| 721 | tgagaacaaa gattatatga tattgttaag tgcgttaaca aattttaaag atgaagagga |
| 781 | aattgtgctt catgtgctgc attgtttaca ttccctagcg attccttgca ataatgtgga |
| 841 | agtcctcatg agtggcaatg tcaggtgtta taatattgtg gtggaagcta tgaaagcatt |
| 901 | ccctatgagt gaaagaattc aagaagtgag ttgctgtttg ctccataggc ttacattagg |
| 961 | taattttttc aatatcctgg tattaaacga agtccatgag tttgtggtga aagctgtgca |
| 1021 | gcagtaccca gagaatgcag cattgcagat ctcagcgctc agctgtttgg ccctcctcac |
| 1081 | tgagactatt ttcttaaatc aagatttaga ggaaaagaat gagaatcaag agaatgatga |
| 1141 | tgagggggaa gaagataaat tgttttggct ggaagcctgt tacaaagcat taacgtggca |
| 1201 | tagaaagaac aagcacgtgc aggaggccgc atgctgggca ctaaataatc tccttatgta |
| 1261 | ccaaaacagt ttacatgaga agattggaga tgaagatggc catttcccag ctcataggga |
| 1321 | agtcatgctc tccatgctga tgcattcttc atcaaaggaa gttttccagg catctgcgaa |
| 1381 | tgcattgtca actctcttag aacaaaatgt taatttcaga aaaatactgt tatcaaaagg |
| 1441 | aatacacctg aatgttttgg agttaatgca gaagcatata cattctcctg aagtggctga |
| 1501 | aagtggctgt aaaatgctaa atcatctttt tgaaggaagc aacacttccc tggatataat |
| 1561 | ggcaccagtg gtccccdaaa tactaacagt tatgaaacgt catgagacat cattaccagt |
| 1621 | gcagctggag gcgcttcgag ctattttaca ttttatagtg cctggcatgc cagaagaatc |
| 1681 | cagggaggat acagaatttc atcataagct aaatatggtt aaaaaacagt gtttcaagaa |
| 1741 | tgatattcac aaactggtcc tagcagcttt gaacaggttc attggaaatc ctgggattca |
| 1801 | gaaatgtgga ttaaaagtaa tttcttctat tgtacatttt cctgatgcat tagagatgtt |
| 1861 | atccctggaa ggtgctatgg attcagtgct tcacacactg cagatgtatc cagatgacca |
| 1921 | agaaattcag tgtctgggtt taagtcttat aggatacttg attacaaaga agaatgtgtt |
| 1981 | cataggaact ggacatctgc tggcaaaaat tctggtttcc agcttatacc gatttaagga |
| 2041 | tgttgctgaa atacagacta aaggatttca gacaatctta gcaatcctca aattgtcagc |
| 2101 | atctttttct aagctgctgg tgcatcattc atttgactta gtaatattcc atcaaatgtc |
| 2161 | ttccaatatc atggaacaaa aggatcaaca gtttctaaac ctctgttgca agtgttttgc |
| 2221 | aaaagtagct atggatgatt acttaaaaaa tgtgatgcta gagagagcgt gtgatcagaa |
| 2281 | taacagcatc atggttgaat gcttgcttct attgggagca gatgccaatc aagcaaagga |
| 2341 | gggatcttct ttaatttgtc aggtatgtga gaaagagagc agtcccaaat tggtggaact |
| 2401 | cttactgaat agtggatctc gtgaacaaga tgtacgaaaa gcgttgacga taagcattgg |
| 2461 | gaaaggtgac agccagatca tcagcttgct cttaaggagg ctggccctgg atgtggccaa |
| 2521 | caatagcatt tgccttggag gattttgtat aggaaaagtt gaaccttctt ggcttggtcc |
| 2581 | tttatttcca gataagactt ctaatttaag gaaacaaaca aatatagcat ctacactagc |
| 2641 | aagaatggtg atcagatatc agatgaaaag tgctgtggaa gaaggaacag cctcaggcag |
| 2701 | cgatggaaat ttttctgaag atgtgctgtc taaatttgat gaatggacct ttattcctga |
| 2761 | ctcttctatg gacagtgtgt ttgctcaaag tgatgacctg gatagtgaag gaagtgaagg |
| 2821 | ctcatttctt gtgaaaaaga aatctaattc aattagtgta ggagaatttt accgagatgc |
| 2881 | cgtattacag cgttgctcac caaatttgca aagacattcc aattccttgg ggcccatttt |
| 2941 | tgatcatgaa gatttactga agcgaaaaag aaaaatatta tcttcagatg attcactcag |
| 3001 | gtcatcaaaa cttcaatccc atatgaggca ttcagacagc atttcttctc tggcttctga |
| 3061 | gagagaatat attacatcac tagacctttc agcaaatgaa ctaagagata ttgatgccct |
| 3121 | aagccagaaa tgctgtataa gtgttcattt ggagcatctt gaaaagctgg agcttcacca |
| 3181 | gaatgcactc acgagctttc cacaacagct atgtgaaact ctgaagagtt tgacacattt |
| 3241 | ggacttgcac agtaataaat ttacatcatt tccttcttat ttgttgaaaa tgagttgtat |
| 3301 | tgctaatctt gatgtctctc gaaatgacat tggaccctca gtggttttag atcctacagt |
| 3361 | gaaatgtcca actctgaaac agtttaacct gtcatataac cagctgtctt ttgtacctga |
| 3421 | gaacctcact gatgtggtag agaaactgga gcagctcatt ttagaaggaa ataaaatatc |
| 3481 | agggatatgc tcccccttga gactgaagga actgaagatt ttaaacctta gtaagaacca |
| 3541 | catttcatcc ctatcagaga actttcttga ggcttgtcct aaagtggaga gtttcagtgc |
| 3601 | cagaatgaat tttcttgctg ctatgccttt cttgcctcct tctatgacaa tcctaaaatt |
| 3661 | atctcagaac aaattttcct gtattccaga agcaatttta aatcttccac acttgcggtc |
| 3721 | tttagatatg agcagcaatg atattcagta cctaccaggt cccgcacact ggaaatcttt |
| 3781 | gaacttaagg gaactcttat ttagccataa tcagatcagc atcttggact tgagtgaaaa |
| 3841 | agcatattta tggtctagag tagagaaact gcatctttct cacaataaac tgaaagagat |
| 3901 | tcctcctgag attggctgtc ttgaaaatct gacatctctg gatgtcagtt acaacttgga |
| 3961 | actaagatcc tttcccaatg aaatggggaa attaagcaaa atatgggatc ttcctttgga |
| 4021 | tgaactgcat cttaactttg attttaaaca tataggatgt aaagccaaag acatcataag |
| 4081 | gtttcttcaa cagcgattaa aaaaggctgt gccttataac cgaatgaaac ttatgattgt |
| 4141 | gggaaatact gggagtggta aaaccacctt attgcagcaa ttaatgaaaa ccaagaaatc |
| 4201 | agatcttgga atgcaaagtg ccacagttgg catagatgtg aaagactggc ctatccaaat |
| 4261 | aagagacaaa agaaagagag atctcgtcct aaatgtgtgg gattttgcag gtcgtgagga |
| 4321 | attctatagt actcatcccc attttatgac gcagcgagca ttgtaccttg ctgtctatga |
| 4381 | cctcagcaag ggacaggctg aagttgatgc catgaagcct tggctcttca atataaaggc |
| 4441 | tcgcgcttct tcttcccctg tgattctcgt tggcacacat ttggatgttt ctgatgagaa |
| 4501 | gcaacgcaaa gcctgcatga gtaaaatcac caaggaactc ctgaataagc gagggttccc |
| 4561 | tgccatacga gattaccact ttgtgaatgc caccgaggaa tctgatgctt tggcaaaact |
| 4621 | tcggaaaacc atcataaacg agagccttaa tttcaagatc cgagatcagc ttgttgttgg |
| 4681 | acagctgatt ccagactgct atgtagaact tgaaaaaatc attttatcgg agcgtaaaaa |
| 4741 | tgtgccaatt gaatttcccg taattgaccg gaaacgatta ttacaactag tgagagaaaa |
| 4801 | tcagctgcag ttagatgaaa atgagcttcc tcacgcagtt cactttctaa atgaatcagg |
| 4861 | agtccttctt catttttcaag accagcact gcagttaagt gacttgtact ttgtggaacc |
| 4921 | caagtggctt tgtaaaatca tggcacagat tttgacagtg aaagtggaag gttgtccaaa |
| 4981 | acaccctaag ggcattattt cgcgtagaga tgtggaaaaa tttctttcaa aaaaaaggaa |
| 5041 | atttccaaag aactacatgt cacagtattt taagctccta gaaaaattcc agattgcttt |
| 5101 | gccaatagga gaagaatatt tgctggttcc aagcagtttg tctgaccaca ggcctgtgat |
| 5161 | agagcttccc cattgtgaga actctgaaat tatcatccga ctatatgaaa tgccttattt |
| 5221 | tccaatggga ttttggtcaa gattaatcaa tcgattactt gagatttcac cttacatgct |
| 5281 | ttcagggaga gaacgagcac ttcgcccaaa cagaatgtat tggcgacaag gcatttactt |
| 5341 | aaattggtct cctgaagctt attgtctggt aggatctgaa gtcttagaca atcatccaga |
| 5401 | gagtttctta aaaattacag ttccttcttg tagaaaaggc tgtattcttt tgggccaagt |
| 5461 | tgtggaccac attgattctc tcatggaaga atggtttcct gggttgctgg agattgatat |
| 5521 | ttgtggtgaa ggagaaactc tgttgaagaa atgggcatta tatagtttta atgatggtga |
| 5581 | agaacatcaa aaaatcttac ttgatgactt gatgaagaaa gcagaggaag gagatctctt |
| 5641 | agtaaatcca gatcaaccaa ggctcaccat tccaatatct cagattgccc ctgacttgat |
| 5701 | tttggctgac ctgcctagaa atattatgtt gaataatgat gagttggaat ttgaacaagc |
| 5761 | tccagagttt ctcctaggtg atggcagttt tggatcagtt taccgagcag cctatgaagg |
| 5821 | agaagaagtg gctgtgaaga tttttaataa acatacatca ctcaggctgt taagacaaga |
| 5881 | gcttgtggtg ctttgccacc tccaccaccc cagtttgata tctttgctgg cagctgggat |
| 5941 | tcgtccccgg atgttggtga tggagttagc ctccaagggt tccttggatc gcctgcttca |
| 6001 | gcaggacaaa gccagcctca ctagaaccct acagcacagg attgcactcc acgtagctga |
| 6061 | tggtttgaga tacctccact cagccatgat tatataccga gacctgaaac cccacaatgt |
| 6121 | gctgcttttc acactgtatc ccaatgctgc catcattgca aagattgctg actacggcat |
| 6181 | tgctcagtac tgctgtagaa tggggataaa aacatcagag ggcacaccag ggtttcgtgc |
| 6241 | acctgaagtt gccagaggaa atgtcattta taaccaacag gctgatgttt attcatttgg |
| 6301 | tttactactc tatgacattt tgacaactgg aggtagaata gtagagggtt tgaagtttcc |
| 6361 | aaatgagttt gatgaattag aaatacaagg aaaattacct gatccagtta aagaatatgg |
| 6421 | ttgtgcccca tggcctatgg ttgagaaatt aattaaacag tgtttgaaag aaaatcctca |
| 6481 | agaaaggcct acttctgccc aggtctttga cattttgaat tcagctgaat tagtctgtct |
| 6541 | gacgagacgc attttattac ctaaaaacgt aattgttgaa tgcatggttg ctacacatca |
| 6601 | caacagcagg aatgcaagca tttggctggg ctgtgggcac accgacagag gacagctctc |
| 6661 | atttcttgac ttaaatactg aaggatacac ttctgaggaa gttgctgata gtagaatatt |
| 6721 | gtgcttagcc ttggtgcatc ttcctgttga aaaggaaagc tggattgtgt ctgggacaca |
| 6781 | gtctggtact ctcctggtca tcaataccga agatgggaaa aagagacata ccctagaaaa |
| 6841 | gatgactgat tctgtcactt gtttgtattg caattccttt tccaagcaaa gcaaacaaaa |
| 6901 | aaattttctt ttggttggaa ccgctgatgg caagttagca atttttgaag ataagactgt |
| 6961 | taagcttaaa ggagctgctc ctttgaagat actaaatata ggaaatgtca gtactccatt |
| 7021 | gatgtgtttg agtgaatcca caaattcaac ggaaagaaat gtaatgtggg gaggatgtgg |
| 7081 | cacaaagatt ttctcctttt ctaatgattt caccattcag aaactcattg agacaagaac |
| 7141 | aagccaactg ttttcttatg cagctttcag tgattccaac atcataacag tggtggtaga |
| 7201 | cactgctctc tatattgcta agcaaaatag ccctgttgtg gaagtgtggg ataagaaaac |
| 7261 | tgaaaaactc tgtggactaa tagactgcgt gcacttttta agggaggtaa tggtaaaaga |
| 7321 | aaacaaggaa tcaaaacaca aaatgtctta ttctgggaga gtgaaaaccc tctgccttca |
| 7381 | gaagaacact gctctttgga taggaactgg aggaggccat attttactcc tggatctttc |
| 7441 | aactcgtcga cttatacgtg taatttacaa cttttgtaat tcggtcagag tcatgatgac |
| 7501 | agcacagcta ggaagcctta aaaatgtcat gctggtattg ggctacaacc ggaaaaatac |
| 7561 | tgaaggtaca caaaagcaga aagagataca atcttgcttg accgtttggg acatcaatct |
| 7621 | tccacatgaa gtgcaaaatt tagaaaaaca cattgaagtg agaaaagaat tagctgaaaa |
| 7681 | aatgagacga acatctgttg agtaagagag aaataggaat tgtctttgga taggaaaatt |
| 7741 | attctctcct cttgtaaata tttattttaa aaatgttcac atggaaaggg tactcacatt |
| 7801 | ttttgaaata gctcgtgtgt atgaaggaat gttattattt ttaatttaaa tatatgtaaa |
| 7861 | aatacttacc agtaaatgtg tattttaaag aactatttaa aacacaatgt tatatttctt |
| 7921 | ataaatacca gttactttcg ttcattaatt aatgaaaata aatctgtgaa gtacctaatt |
| 7981 | taagtactca tactaaaatt tataaggccg ataatttttt gttttcttgt ctgtaatgga |
| 8041 | ggtaaacttt attttaaatt ctgtgcttaa gacaggacta ttgcttgtcg atttttctag |
| 8101 | aaatctgcac ggtataatga aaatattaag acagtttccc atgtaatgta ttccttctta |
| 8161 | gattgcatcg aaatgcacta tcatatatgc ttgtaaatat tcaaatgaat ttgcactaat |
| 8221 | aaagtccttt gttggtatgt gaattctctt tgttgctgtt gcaaacagtg catcttacac |
| 8281 | aacttcactc aattcaaaag aaaactccat taaaagtact aatgaaaaaa catgacatac |
| 8341 | tgtcaaagtc ctcatatcta ggaaagacac agaaactctc tttgtcacag aaactctctg |
| 8401 | tgtctttcct agacataata gagttgtttt tcaactctat gtttgaatgt ggataccctg |
| 8461 | aattttgtat aattagtgta aatacagtgt tcagtccttc aagtgatatt tttatttttt |
| 8521 | tattcatacc actagctact tgttttctaa tctgcttcat tctaatgctt atattcatct |
| 8581 | tttccctaaa tttgtgatgc tgcagatcct acatcattca gatagaaacc tttttttttt |
| 8641 | tcagaattat agaattccac agctcctacc aagaccatga ggataaatat ctaacacttt |
| 8701 | tcagttgctg aaggagaaag gagctttagt tatgatggat aaaaatatct gccaccctag |
| 8761 | gcttccaaat tatacttaaa ttgtttacat agcttaccac aataggagta tcagggccaa |
| 8821 | atacctatgt aataatttga ggtcatttct gctttaggaa aagtactttc ggtaaattct |
| 8881 | ttggccctga ccagtattca ttatttcaga taattccctg tgataggaca actagtacat |
| 8941 | ttaatattct cagaacttat ggcattttac tatgtgaaaa ctttaaattt atttatatta |
| 9001 | agggtaatca aattcttaaa gatgaaagat tttctgtatt ttaaaggaag ctatgcttta |
| 9061 | acttgttatg taattaacaa aaaaatcata tataatagag ctctttgttc cagtgttatc |
| 9121 | tctttcattg ttactttgta tttgcaattt tttttaccaa agacaaatta aaaaaatgaa |
| 9181 | taccatattt aaatggaata ataaaggttt tttaaaaact ttaaaaaaaa aaaaaaaaa |

For example the polypeptide sequence corresponding to human LRRK2 is encoded by the nucleic acid sequence of SEQ ID NO: 10 and is depicted in SEQ ID NO: 11 (2527aa). Sequence information related to LRRK2 is accessible in public databases by GenBank Accession numbers NP_940980.3 (protein).

| | |
|---|---|
| 1 | MASGSCQGCE EDEETLKKLI VRLNNVQEGK QIETLVQILE DLLVFTYSEH ASKLFQGKNI |
| 61 | HVPLLIVLDS YMRVASVQQV GWSLLCKLIE VCPGTMQSLM GPQDVGNDWE VLGVHQLILK |
| 121 | MLTVHNASVN LSVIGLKTLD LLLTSGKITL LILDEESDIF MIIFDAMHSF PANDEVOKLG |
| 181 | CKALHVLFER VSEEQLTEFV ENKDYMILLS ALTNFKDEEE IVLHVLHCLH SLAIPCNNVE |
| 241 | VLMSGNVRCY NIVVEAMKAF PMSERIQEVS CCLLHRLTLG NFFNILVLNE VHEFVVKAVQ |
| 301 | QYPENAALQI SALSCLALLT ETIFLNQDLE EKNENQENDD EGEEDKLFWL EACYKALTWH |
| 361 | RKNKHVQEAA CWALNNLLMY QNSLHEKIGD EDGHFPAHRE VMLSMIMHSS SKEVFQASAN |
| 421 | ALSTLLEQNV NFRKILLSKG IHLNVIELMQ KHIHSPEVAE SGCKMINHLF EGSNTSLDIM |
| 481 | AAVVPKILTV MKRHETSLPV QLEALRAILH FIVPGMPEES REDTEFHHKL NMVKKQCFKN |
| 541 | DIHKLVLAAL NRFIGNPGIQ KCGLKVISSI VHFPDALEML SLEGAMDSVL HTLQMYPDDQ |
| 601 | EIQCLGLSLI GYLITKKNVF IGTGHLLAKI LVSSLYRFKD VAEIQTKGFQ TILAILKLSA |
| 661 | SFSKLLVHHS FDLVIFHQMS SNIMEQKDOQ FLNLCCKCFA KVAMDDYLKN VMLERACDQN |
| 721 | NSIMVECLLL LGADANQAKE GSSLICQVCE KESSPKLVEL LLNSGSREQD VRKALTISIG |
| 781 | KGDSQIISLL LRRLALDVAN NSICLGGFCI GKVEPSWLGP LFPDKTSNLR KQTNIASTLA |
| 841 | RMVIRYQMKS AVEEGTASGS DGNFSEDVLS KFDEWTFIPD SSMDSVFAQS DDLDSEGSEG |
| 901 | SFLVKKKSNS ISVGEFYRDA VLQRCSPNLQ RHSNSLGPIF DHEDLLKRKR KILSSDDSLR |
| 961 | SSKLQSHMRH SDSISSLASE REYTTSLDLS ANELRDIDAL SQKCCISVHL EHLEKLELHQ |
| 1021 | NALTSFPQQL CETLKSLTHL DLHSNKFTSF PSYLLKMSCI ANLDVSRNDI GPSVVLDPTV |
| 1081 | KCPTLKQFNL SYNQLSFVPE NLTDVVEKTE QLILEGNKIS GICSPLRLKE LKILNLSKNH |
| 1141 | ISSLSENFLE ACPKVESFSA RMNFLAAMPF LPPSMTILKL SQNKFSCIPE AILNLPHLRS |
| 1201 | LDMSSNDIQY LPGPAHWKSL NLRELLFSHN QISILDLSEK AYLWSRVEKL HLSHNKLKEI |
| 1261 | PPEIGCLENL TSLDVSYNLE LRSFPNEMGK LSKIWDLPLD ELHLNFDFKH IGCKAKDIIR |
| 1321 | FLOQRLKKAV PYNRMKLMIV GNTGSGKTTL LQQLMKTKKS DLGMQSATVG IDVKDWPIQI |
| 1381 | RDKRKRDLVL NVWDFAGREE FYSTHPHFMT ORALYLAVYD LSKGQAEVDA MKPWLFNIKA |
| 1441 | RASSSPVILV GTHLDVSDEK QRKACMSKIT KELLNKRGFP AIRDYHFVNA TEESDALAKL |
| 1501 | RKTIINESLN FKIRDQLVVG QLIPDCYVEL EKIILSERKN VPIEFPVIDR KRLLQLVREN |
| 1561 | QLQLDENELP HAVHFLNESG VLLHFQDPAL QLSDLYFVEP KWLCKIMAQI LTVKVEGCPK |
| 1621 | HPKGIISRRD VEKFLSKKRK FPKNYMSQYF KILEKFQIAL PIGEEYLLVP SSLSDHRPVI |
| 1681 | ELPHCENSEI IIRLYEMPYF PMGFWSRLIN RLLEISPYML SGRERALRPN RMYWRQGIYL |
| 1741 | NWSPEAYCLV GSEVLDNHPE SFLKITVPSC RKGCILLGQV VDHIDSLMEE WFPGLLEIDI |
| 1801 | CGEGETLLKK WALYSFNDGE EHQKILLDDL MKKAEEGDLL VNPDQPRLTI PISQIAPDLI |
| 1861 | LADLPRNIML NNDELEFEQA PEFLLGDGSF GSVYRAAYEG EEVAVKIFNK HTSLRLLRQE |
| 1921 | LVVLCHLHHP SLISLLAAGI RPRMLVMELA SYGSLDRLLQ QDKASLTRTL QHRIALHVAD |
| 1981 | GLRYLHSAMI IYRDTKPHNV LLFTLYPNAA IIAKIADYGI AQYCCRMGIK TSEGTPGFRA |
| 2041 | PEVARGNVIY NQQADVYSFG LLLYDILTTG GRIVEGLKFP NEFDELEIQG KLPDPVKEYG |
| 2101 | CAPWPMVEKL IKQCLKENPQ ERPTSAQVFD ILNSAELVCL TRRILLPKNV IVECMVATHH |
| 2161 | NSRNASIWLG CGHTDRGQLS FLDLNTEGYT SEEVADSRIL CLALVHLPVE KESWIVSGTQ |
| 2221 | SGTLLVINTE DGKKRHTLEK MTDSVTCLYC MSFSKQSYQK NFLLVGTADG KLAIFEDKTV |
| 2281 | KLKGAAPLKI LNIGNVSTPL MCLSESTNST ERNVMWGGCG TKIFSFSNDF TIQKLIETRT |
| 2341 | SQLFSYAAFS DSNIITVVVD TALYIAKQNS PVVEVWDKKT EKLCGLIDCV HFLREVMVKE |
| 2401 | NKESKHKMSY SGRVKTLCLQ KNTALWIGTG GGHILLLDLS TRRLIRVIYN FCNSVRVMMT |
| 2461 | AQLGSLKNVM LVLGYNRKNT EGTQKQKEIQ SCLTVWDINL PHEVQNLEKH IEVRKFLAEK |
| 2521 | MRRTSVE |

The invention provides for a nucleic acid encoding a VPS35 protein, or fragment thereof.

For example, the human genomic nucleotide sequence corresponding to the sense strand of the human VPS35 gene is depicted in SEQ ID NO: 12 (29556 bp). Sequence information related to VPS35 is accessible in public databases by GenBank Accession number NG_029970.1 (nucleotide).

SEQ ID NO: 12:

| | |
|---|---|
| 1 | gctagagagg gcggggcttg gaggggccgc agcgtcacat gaccgcggga ggctacgcgc |
| 61 | gggccgggtg ctgcttcctg caggctctgg ggagtcgcca tggtgagtgc tgagggggca |
| 121 | gtggcacctg ggtcgaccct ccttgtagcc cctgctctct cccaccgccc cgcactccag |
| 181 | cgagtggaga aggggcccca cagaccgttc gggattaaga ccagcccgat ttggcctgcg |
| 241 | ggatagggga cagcaggagg aaggccgcgg gcaggctgat ccgggccggg gtgggcggcg |
| 301 | gctcttggct gcggccgttg ctgagagacg gggcggcctc tctgtggggt tgacttggca |
| 361 | tgtaggcttt ggggtccatg aaggcctgcg gcctccttta agtggaatcg gtcacctgcc |
| 421 | taccacgagg ggaccggtag tcctaggtct gagcgtctgg cccccggggc gcgtggaggc |
| 481 | cctgagactc ggaggtggcg ccggcacccg cccagatgtt gcgtttctac ctttgtgcct |
| 541 | agttgtgctc ggccgtcccc acgccctcct ggaggggtcg cagtgattcc ttggcctttc |
| 601 | ttggcctcat acccgccttc ggctgcagtg tttgtcagcg agttctgggg acctgcttac |
| 661 | atgaatttcc tggaaggact caggctgtct tctaatcctg acggtcgcaa aggagactga |
| 721 | ttgtttactt tagcatttgt gcattgggcg caccttgcct cttttgtctc gccattgata |
| 781 | aaatccaagt atttgacttg ctggaagcag tacttctcct tagggcccgt ctatgacggc |
| 841 | agcaaatcgt ggtgtggctg ttcgccggta aacttgaact tcctcaaaat gtgaatcttt |
| 901 | gtgtctggtt cccacaaagg caagttgtca cttgcatttt attagcgttt aacatagcct |
| 961 | gcactgtgta aataaatttt ttgagtatat actgtatgtc cgctttaatt accttactca |
| 1021 | ctctgtgtag ataggcttct gtaaatctgt aagcctggaa acagatttca ctttaaatgt |
| 1081 | cttaatgcca gaaaggatta agtgttttac aaatactatt ttcatataac gtgttgccgt |
| 1141 | acaaggtgat tttgcctgtt tctcaggatt tttataattg ggaattgata caagaccggc |
| 1201 | gcaaaattta actttaggat ttgtgtgttt ccagcgttta tggattgaca tttatattgt |
| 1261 | tttgtaatgg aaaacactta attgaggatg tattacacac tccgattctt tgttgggtga |
| 1321 | accagttggg agcaatcagc cagacacaca gtcttgtcct catgaatttt attgggaaag |
| 1381 | gaaacttgag tattcgttct ttcacagtca ctgtctataa tttaggcagc tcgcttagtc |
| 1441 | ttcttggtgt cgcacttttg catccgaaag caagtgttgg cggccccttt ggcccttccc |
| 1501 | agccctaaaa ttccagaatc cacttcaatt catcaaacat cttgtgtgcc ttcagtgtca |
| 1561 | cggactgtgg ccctgggtac caatgtcgaa cttaagaggc tactggggga gacacgcttg |
| 1621 | tagaacttaa ctgtaataca gtgtgtggat tgctttacta acattgtcaa ctcagtgctt |
| 1681 | tggggtcatg gagggatagt tttgcctggg gattttggga aggactacag gaagtaacgt |
| 1741 | gaactgagac tataaagagg gtggaaagaa attctccagg tggaaagaaa aggtattgct |
| 1801 | aatagatgaa gccaaaacgt tcagtgtgct ccatcagagg attcttgggg atatgtattg |
| 1861 | gaaatgaagt tagagccgtt tggtgttcaa attatatttt gaagttgtca tgccctcatc |
| 1921 | atccttattt ctctctactt cagttgtcaa tgtatgccat gtcatcattt ttaaaatagt |
| 1981 | tctttcatta acgtgatatt ctgtaccttt gtttgaagaa atgagcgtta catttggttt |
| 2041 | acattgtact gtcagattac atctacagca tataagcagg gaggttgaac cacaataggt |
| 2101 | ttctaacata agtggagata attatttgta tctaaactga ataataatta tagcacttat |
| 2161 | taagtgctta ttgctaagta gtaggcacgt tttgagcttt ggtttcattt gttcttcata |
| 2221 | acagtggtgt actctcatct ctgtttcata gaagaggaaa gaaaagcaca ttgtttcagt |
| 2281 | ggagagtagt tttccaagtg atcagttcat gatctttgct ttgctctgca cattagaact |
| 2341 | gtagacttga gagggctgtt tgtgggtctc aagctaaaat gggactaatc acaaatgtta |
| 2401 | atttgattcc cattctgtag ggtttccatt ttcttctgat tcatgtagct gtgaagtacg |
| 2461 | gtcatttaca aatggaaaac cactttcatg attgaggaaa cattggttaa ttggcttgtg |
| 2521 | tttaatgata gcgtgctcat attataactg ttaaggctct atgtgacact tcatagtaag |
| 2581 | gcatcaagaa tagcccttat aatagctgtt gctagcataa ctacactgtt ttatgagtaa |
| 2641 | tatataaaaa tagattgctt tactatagct ttatgtcttc acttgtagct tactttgtaa |
| 2701 | taagtcatat tctgtaatct tcaacatttt gtatttacat ttctatttta agctgagttg |
| 2761 | agacaaaata gaagaatttt cctaaaattg cattcttttg tttacacatc tctaaatctt |
| 2821 | tctagtcatt ctatacaaat gtttttggaa ctgattgtat cctctttgta gccacagttt |
| 2881 | catttgctat ataataaaaa ttatcataca caacagccaa aaggtggaaa caacccatat |
| 2941 | gtccatggat aaacaaaatg tagtgtgtgt gtatatatat agatatataa tgaaatactt |
| 3001 | cgtttttaaa aggagggaaa ttctggcaca tactgcaaca tggatgaaca ttgaagatat |
| 3061 | tatggtcagt gaaataagcg agacataaaa gtaaaaatat tgtatgattt cacttaaatg |
| 3121 | aagtatatag agtagtcaaa ctcagagaaa agaaggtaga attgcctggg ggttcggaat |
| 3181 | gggagtttaa tggctacagt ttctatttgc atttgtaaag ataaaaagtt ttagagatag |
| 3241 | atggtggtaa ttgtattaat gtgaatgtac ctaatgccac tgaactgtac atttaaatgt |
| 3301 | taaaatggta aattttaggt acattttacc acaacaagaa gtcattttta attaaaaata |
| 3361 | tccagatgta tcataaagaa aaataaaaaa aattcagatg tatcactgtt tatctctaaa |
| 3421 | tggatcaatt gaacttaatg aaatccattg attcaaatta ttattaaatc tattgcgtcc |
| 3481 | agaggtaagg agccaaaaaa ttccaaatga tggcctggtt tcactaaagt tcagagaaga |
| 3541 | ctagcccatg atgaatagta aatttcatta agtcagagtc tttaaatgct ggtgtcatcc |
| 3601 | ttgcctctga aaccagcatt ttatggtaat agttccactg ggttaaattc atgttccctt |
| 3661 | taagtgaagt ttaaaagata cctaacttct tctttgaaat ttgtttgtgc ttctgaggaa |
| 3721 | gagtgcttgc agcagagctc agtttactag agtttttcat agggaaaaaa agggagatgc |
| 3781 | atggtgttgt tcattattca gttaatattt ttctctttcc aaagttagaa caagagaaag |
| 3841 | ttttcaattt ttataagcta tgctagttca gaagtgggtt ttatgttatc aagtttcttt |
| 3901 | gttaatctca aaatgaaatg ttgttttgct tttcttagat aatgaaacag accagatttt |
| 3961 | acttgcaggt tgatgtgtaa gtccttgcct tccacctctt caactcattg tgtgagaggc |
| 4021 | attttgtctt tagtcattgt tttaaaaaat aaaagtgaaa tgacataaca aaaaattaac |
| 4081 | cattttaaag tgaacaattt ataaagtggc atttagtatg ctcacaatac tgtacaatca |
| 4141 | ccacctttgg tttcaaaaca ttttccctac ccctaaaaga aagccagtgc ccattaagtg |
| 4201 | attattcccc tcagcctctc tcctggtaat catcagtcta ctttctgttc ctatacagaa |
| 4261 | tatttcatat tagaggaatc atacaatatg tggcttttta tgtctagctt ctttcattta |
| 4321 | gcatgatgtt ttctaggttt gtttatgtag caatacttca ttccttttca tggttgaata |
| 4381 | atattctgta attgtatgta tataccacaa cttgtttatt catccattaa ttattcagtc |
| 4441 | tttttttgtt aaatatctaa acattctaaa accagtgtat tcatttatgc agtgaacatt |
| 4501 | tgtggagcat attatgtatc aagcagtgtg ttggatccca aggatgtaaa aatgaccgtc |
| 4561 | atttataata ataatgtgat acatgctgtg gtggagatgt aaacagtgtt tatatgactt |
| 4621 | gaagaagtgg ttaattcttt ttggcatgga gattgaattc attttgcaag cagatttttg |
| 4681 | ctgatggcaa agagaagagt gtctagatat attttgtcca ctgtagctgt agatcagtat |
| 4741 | atctgaaatg gtgtaagcta tttgaggaca gtgctcttta ttgttttcta ctgtaagtca |
| 4801 | ctaacaattt tggctgtttt atttctagac tgtttagtct tttgttaaat attgccaagg |
| 4861 | aggggctggt cactgttctc gtaaactagt tccttagtct gtcttaagaa tagactgaaa |
| 4921 | tgcagatgat aagtagtcta gaggaaaaga gaggctttag agattggttt cggctatacc |
| 4981 | tatcacaaga tttcgattgg tcagatggct atgtctgggt tggattcaga gtgtgttagc |
| 5041 | agaacacagc catgaactac cactgcaagt ttctttgagg ccagcctact ttctgagaga |
| 5101 | gaggcaattc tttgtacaca tactattctc ctttgtcagt cttattctgt taacttcagc |
| 5161 | gataaggcat gactctgtgt gcagcagctg ttaataattg gtaaatgggc tgggtgcggt |
| 5221 | ggctcatgcc tataatccaa gcactttggg aggtcgaggc aggcgaatca cgaggtcagg |
| 5281 | agatggagac tatcctggct aacatggtga aaccccgtct ctactaaaaa tagaaaaaaa |
| 5341 | attagccggg tgtggtgatg ggcgcttgta gtcccagcta cttaggaggc tgaggcagga |
| 5401 | gaatggcttg aacccgggag gcggagcttg ccatgagccg agatggcacc actgcactcc |
| 5461 | agcctgggcg acagagctag actccgtctc aaaaaaaaaa tttggtaaat gggtttgagg |
| 5521 | tatagagctg gacattgttg gagaaggact atggctagaa ctatagaaat aacgtacttg |
| 5581 | ctagaagaat gtgcttgaga catcagtgga attttttatt tttcagccta caacacagca |
| 5641 | gtcccctcag gatgagcagg aaaagctctt ggatgaagcc atacaggctg tgaaggtcca |
| 5701 | gtcattccaa atgaagagat gcctggtaag aatggagatg tgggaggcac agttgcagtt |
| 5761 | cgtgtgttcc taaggaagca tgtgcagtgt cttctagagt caggtgtttc tggtaaatct |
| 5821 | aatcttcacc gtttaccagc atctatcttc agtctcatct ccctcaagca ctttgtggag |
| 5881 | caatttcaac aaagagccct gtttactcac atgtatattt atggtttggg attgtctgtc |
| 5941 | ttccctacta gaatacaagc tcataagaat aagagaccct tccttttatt tacacattac |
| 6001 | tgtattatta ccacaccagt gtctgaccag aattactagc ctccttggtt ctatacctca |
| 6061 | gacctgagga atatttaaca tataataggt actcagtaaa tatttgttga atgaatggat |
| 6121 | ttaaatgctt tgcatttgaa ttattcagct ttttttctaa atatcttgaa aactttaatt |
| 6181 | tctttgctga atagatatat ttattgtaga agctagctta aaaattatac ttaacactta |
| 6241 | tttacatatt tttatattct aaaagataaa gtaagagata atctgtgtag atacttttga |
| 6301 | ttctctggat taaaatgtaa ggaattgagc caaattggtt agtactttaa actataaatt |
| 6361 | actgtgatga agatgatgct attttacctt tgtaaaatgt cttactgtgc tttctaaagc |
| 6421 | atagtaatat gctcttgtgt cttttattgg tttaattcct aacaaattgg gaatgaaaaa |
| 6481 | taaatgtctt ggaatggaga agctgggttt gctattgctt gcttctttct cttcctgtgt |
| 6541 | atggatagtg tttcctctat ctcaaggaat tgcttgcatt tctgagttaa gtggaacata |
| 6601 | tgggcattgt gagggcttga agaatgcaag aggaaagcaa acttacatgg atagtcattt |
| 6661 | cagacagctc tgaagagtct ttaacccatg acaaagccat gtcaggatag tatcttcctt |
| 6721 | cacctgaatc agtatgccag ttctcttgat tgcaggtaaa atgtgatgaa tggagctagt |
| 6781 | ttcctagtct ctatagattg aaaagattag cattctatca agaagcttgc agtcttagct |
| 6841 | atgttaagtc ttactaagaa tcatgtatct ttttcttttt cagtagagac ggcaaggtga |
| 6901 | accgatctaa gttgtttttt taatgtggtt aaaatcattt aagtgcggta ttcttttaaa |
| 6961 | actatgtaac aagtccttga tgtaaagaat ttgtacaacc aagataaatg tttatttaaa |
| 7021 | ttaagcattc tcatctattc tcttggtatt tctgtaggac aaaaacaagc ttatggatgc |
| 7081 | tctaaaacat gcttctaata tgcttggtga actccggact tctatgttat caccaaagag |
| 7141 | ttactatgaa ctttgtatct tttgaatgtt gaagactaaa catttggacc ataccttttt |
| 7201 | cttgataagg cctattttgt ttgttcttta tgaagttttt ctggagttat cttattcttc |
| 7261 | gttatctgag tcacatggca ctccttctcc atgcagatgt gctaagtgag aaaaacactt |
| 7321 | tgagagtact cctttcctat gcttaaacat ctttaaatgt gttgtcggtg catctcaatt |
| 7381 | ttcagaccct tcatgaggat atttaggcta tgacacagtt ggttctttaa tacttagatt |
| 7441 | ttgttatgca gcagtctcaa atggacagga atttaatcat ttgccatttc aaaacccatt |
| 7501 | agcagtctga caggtaacca ttgtatttac tgctttgctt gaccacacat gctttaaaac |
| 7561 | ccttatttta aagtaagaaa agtccggcta aaattcatcc ttcgcttgaa cactttctta |
| 7621 | aaggactaaa acttaagatg tctgcccagt agttagtaat gactccaaca agtttcaaag |
| 7681 | ttttgtttag gttggcttat ttttattttt agtccttaat cataattaaa agatatggcc |
| 7741 | atttctgatg aactgcacta cttggaggtc tacctgacag atgagtttgc taaaggaagg |
| 7801 | aaagtggcag atctctacga acttgtacag tatgctggaa acattatccc aaggctgtaa |
| 7861 | gtaattacaa atcagagaac ttttgtgtct gtatttctca ctatatgtta cgtcttttat |
| 7921 | gattatcagc ttaagaaaaa gttttaaggg taacttctta acaaattgag atgaacattt |
| 7981 | tggtagatat tctcttactt gttttagagt aactagattt acgttttatg tagatatttg |
| 8041 | aggaattttg gaaatagaaa aaatggacat gcttgctatt ttttttaatg tcttgactat |
| 8101 | tagaaaaatt aatataattg ttctcttcct aatatgttta aaggtaatat ctatgttgta |
| 8161 | tatatacagt gtgtgtgtgt gtgtgtgtgt gtgtgtgtgt atagtttttt tagaggtcag |
| 8221 | tcagtggtta tattttaaat gagatatttt ccttgtcatg cgggagaaaa caacatggtt |
| 8281 | cctgtcttgt ttatttaatg ttttgttcag tgtgtttgga aataaattct tgatttgaat |
| 8341 | attttatttc taatcagcat ttcttcataa ttttcctagt taccttttga tcacagttgg |
| 8401 | agttgtatat gtcaagtcat ttcctcagtc caggaaggat attttgaaag atttggtaga |
| 8461 | aatgtgccgt ggtgtgcaac atcccttgag gggtctgttt cttcgaaatt accttcttca |
| 8521 | gtgtaccaga aatatcttac ctgatgaagg agagccaaca gagtaagtga ttttctttct |
| 8581 | taattttgtt gcaatatttc tttcattgta gaatgtataa aagtgtggaa acatatacag |
| 8641 | aaacaaagtg tgaataattc ttccacccag tcagccattt aggtagcatt tgtatataga |
| 8701 | tttcctttgt aatatagaac tcctcagtat atgtggtatc atctaaaatg tactcttatg |
| 8761 | caaattttat ctttggattg ttaggacctg cttttttcat ttaatgtaat tttttctact |
| 8821 | acattaaatc ttctttgaaa ataaaacttt tttaagagag ttgtatttgg aaattgaatt |
| 8881 | tgtaatgaaa taataaagtg tgagccagct ggatttcata attgttcctt tagtgtctat |
| 8941 | cagtttttat aatttataga ctgctagtta ccttggaata taagtgattt gaattatctg |
| 9001 | ttacgagtta gctattaact ccagagaagc aaaaataaaa gccattcaga gacactcctg |
| 9061 | tctcttgtgt tatcagtatt ctagcatcaa agtctactgt acttttatcc cacagcaggg |
| 9121 | gcagatggtc agccaactgt ggtcttcagt ggggtgagct gttcacatga caggtcccca |
| 9181 | gattaaagaa cttcattcct tttttaaaaa gtttattcat ttattttctt tcttttttta |
| 9241 | atttttaatc ttttttcagt ttgccccaac agattttgtt tttttctttt taatattttc |
| 9301 | atttatttct aaggttttta atatatcatt tatttctaat gttttttaat atatctgcat |
| 9361 | caatttcttt taaaacagta cagaaaagat aaaacattta acaatgtaga gaaattgatg |
| 9421 | aagttacttg ctttattatg ttttgagtgt ccgttttgag catttaatta ggcaatcaat |
| 9481 | aacaattttt gaaaggtact gaggtctcca tcctaggaga cgtagaaaaa taaagcagga |
| 9541 | aatccatggt ctcttccctc acaaagctta cattccaatt aaaaacaaaa tattcaacag |
| 9601 | taaaatgatg tagttagcag tacaccataa gtgttacatt ttttagcctt ttgtttttgt |
| 9661 | ttttggtttg tggggatggg gtctcattat gttgcccagg ccagttttaa actcctggcc |
| 9721 | taaagcgatc ttcctgcctt ggcctcccaa agcactggat tacggacatg agccaccatg |
| 9781 | cccatccttc tagccttttt caattaagga agttgccata agagcaagtc cagttggccc |
| 9841 | aggatgagga gttggggaaa gtaatttgcc ctttaaattt atactgtcct ctccatggta |
| 9901 | ctttttaccc tagagtctgt tccctttgaa atttaacact aagccaatga agttgaaagt |
| 9961 | gattttttat aaagcattgg tgtactatag agataagtag gaaatacaca aaggagaagg |
| 10021 | atagtagtaa gttggtcctg taaatactgt gtaaagactt ttctgtttct ttgcagtgaa |
| 10081 | gaaacaactg gtgacatcag tgattccatg gattttgtac tgctcaactt tgcagaaatg |
| 10141 | aacaagctct gggtgcgaat gcagcatcag ggacatagcc gagatagaga aaaaagagaa |
| 10201 | cgagaaagac aagaactgag aattttagtg ggaacaaatt tggtgcgcct cagtcagttg |
| 10261 | gaaggtgtaa atgtggaacg ttacaaacag gtttatatat ttttgttacc tcttcttatg |
| 10321 | ttcagagata aactgaaatc tgatttttaa aatcagaata tttttgttat acaatagtac |
| 10381 | attgaaaaac atcttaaaat ggctgttatt gaagaagact taaacggaaa gatatatatg |
| 10441 | cagtgtttgt ggattggaag acttaatatt gtcaaaatag catttaaaaa gaattgattt |
| 10501 | atagatccaa tgtaatctca gtcaaaatcc cagcagactt ttgtagaaat taagaagctg |
| 10561 | attctaaagt ttatatgaag aaacaaagaa cctggaacag ctacaacgaa tttgaaaagg |
| 10621 | aagaacaagt tgaaagaccc aagcaacctg aattaatgat ttactctaaa gctgcagtga |
| 10681 | gatcctgtgt ggtattggtg aaaaggatag acacacaaat caatggaggg gaataaaaca |
| 10741 | gggaatggca aactttacct gtgagggacc agataataaa tggtttttgg ctttgtaaga |
| 10801 | catgtgctgt acaacaagct tgtccaacct gcagcccagg acagccttga atatggccca |
| 10861 | acataagttt gtaaacttta aaacatgaga ttttttgctt tttttttttt tttttttttt |
| 10921 | tttttttaaa gctcatcagc taagtgtatt ttatgtatgg cccaagacaa ttctaattct |
| 10981 | tcttcaagcc aaaagattgg acaccctagt ctacaactaa taacagtgca gatatggtgc |
| 11041 | aaaagcaccc acaggcaata tggaagtgaa tgggcatggc tgtgttctag taaaacttta |
| 11101 | tttgtaaaaa caagcagcag ctcagtttac cgatctctga ctggacaatc cataatagac |
| 11161 | ccagatattt atgatcagtt atttttgata aaagtacaaa ggcaaccttt tcagcaagtg |
| 11221 | attctggaac aattggatgt ttatatgcaa acaaaaaacc ctgaaccttg acccatccct |
| 11281 | cataccatat agaaaaaaca cagaaatcaa tcagagacct aaatatagaa cctaataatg |
| 11341 | ttagaagaaa acacagagga aatctttatg acctaggatt agacaaagat ttctgaggat |
| 11401 | atacaagcac aagccatgaa gaaaaaagct cacttttgag aggccaaggc agatggatca |
| 11461 | cttgagtcca ggagtttgag acaggcctgg gcaacatagg gagaccccat ctctacaaaa |
| 11521 | attaccaaaa ttagctgggc atggtggaac gtacctgtag tcccagcact caggaggctt |
| 11581 | gaggtgggag gatgacttga gcctaggagg tggatgttgc attgagtgga gattgtgcca |
| 11641 | cttcactcca gcctgggcaa ccgaacaaga ccttgtctca aaaagaaaaa agcttttaaa |
| 11701 | gtttagaagt gaagtcttgg tgagaaaaat ctcaaatacg attttcaagt tagtagttca |
| 11761 | aatgcgttac tagaggaata gcttaagatt ttgaaaacag attttaaccc ttatgtgtgt |
| 11821 | tttttctctt ttagattgtt ttgactggca tattggagca agttgtaaac tgtagggatg |
| 11881 | ctttggctca agaatatctc atggagtgta ttattcaggt agctgggaac atttcatttt |
| 11941 | tttttaaacg acctatttta tctttcatta aatttaattg ttttgaaaaa attttgatgg |
| 12001 | aataggaaat aagctttcct gaataaagag ttttccttgc ggggtgtggt gactcacacc |
| 12061 | tgtaatctca gcagtttggg agttcaaggt gggaggatct cttgaggcca ggagttcaaa |
| 12121 | accagcctgg gcaacatagc acgatgccgt ttctataaaa aattaaaaaa atttttttag |
| 12181 | tgtttctttt ttttttcatg taatcttgct tcttctaaaa ataatttaaa aataggaatt |
| 12241 | ttctgtttct aacttatacc ttggtctttg tatcaatgtg gtttgttttc ctccaaaatg |
| 12301 | taggaatgag taatctgagt tttctaggtc tctgtagctt tagtttaatt gtaggtgcac |
| 12361 | tttgtttatt ggaatatttc tgtctgagct tatgtttagt agagaggttc aaaagtaatg |
| 12421 | tgtttgaatt tagttgtata agaatacagt gtttttttcc cacaaatgtg aactttacca |
| 12481 | tatgtgagtc cagaatatta cgtgaaatac ttttatttgt attgatcatt tgattttcag |
| 12541 | gttttccctg atgaatttca cctccagact ttgaatcctt ttcttcgggc ctgtgctgag |
| 12601 | ttacaccaga atgtaaatgt gaagaacata atcattgctt taattgatag gtaagacctt |
| 12661 | ccaacactgg cggataaatg ctctgacttg ggaataatga attttaaaca tttttttgaa |
| 12721 | ttatttgttt ctgttacatc tttatcatac caatgatctt aatttaatta tactataaat |
| 12781 | aatttagctt tgtgagtatg agtactaggt acttgtctag gttagacatg aaagaggctt |
| 12841 | aacttaaatg tgcaggagac gtgaagataa tgaatatctt tattctgtgt gcttaattga |
| 12901 | catttaaaga tgttgtacag acttattttt taaatcatac aaatccaaag atcatattga |
| 12961 | agaacaaaat ttgtttttta ccatgatgta agtatcttgc agtgggaact catttgattt |
| 13021 | agagtagccg taagatactg atgattgaaa atgttcaagt aatcactcta tcatcacatt |
| 13081 | ttcttaaaga aaaaatttta agtatcaaat atgtttagta catccacttt tttattttct |
| 13141 | taggtttttt tttttttttt tttttttgag acagatcctc actcttgtca cccaagctag |
| 13201 | agtgcagtga cgctgtctcg gctcactgca acctctgact cctaggttca agtgattcta |
| 13261 | gtgtctcagc ctccggagta gctgggatta cagacatgca ccaacaagcc cagctaattt |
| 13321 | ttgtattttt agtagagaca gggttttgcc aggttggcta ggctagtctc aaactcctga |
| 13381 | gctcaaatga tctgcctgcc tcagcttccc aaagtgctgg gattacagac atgagccact |
| 13441 | gcgcttggcc aatgggtggc ttttttgcag ccatgttatg tagtagtata tgatgtctgt |
| 13501 | cctacacttg taagcattgt catgaaacca gaaacctaag agaagattta tttctgcaga |
| 13561 | taccttttgt atgtttttta aaaaactaag ttattagttt taaagtctga gaatttagat |
| 13621 | aacaaatttt tccaaattgt cagctcaatc ctgggcagca aaaattccat acttattggg |
| 13681 | cccactctta aaggaagcta gtaactggat tttcctgagt tgcctgtaat gtcacttaca |
| 13741 | catctctgtc agtagtgatg cttctgggca tagcaaaatg tggatgtagt tgtgactgac |
| 13801 | aaacagataa tgataatgaa acatactatt ttgagtaatt taagatgtgg gaaataaaag |
| 13861 | ttaattttat gaattttaga cttagttgta tttcaagctt tagtaaaaat gcagtatctt |
| 13921 | aaaatagtct atgtactttt attttttaaa ggttatttat ttaaatcatg gttgttgaat |
| 13981 | acatttgtca ctttaatgca tttctgtcca tatctgctta attatgcttc aaagagttga |
| 14041 | gagaattatc ttgttgaaaa tctacttaat atggtgtgaa ataagaatgc tgatgaaaaa |
| 14101 | ggtttcattg gcaaaactgt ttagttaaaa atgaattgag gaggccgggt gcagtggctc |
| 14161 | acatctgtat tcccagcact ttgggaggcc aaggagggag gcttgcttga gtccaggtca |
| 14221 | gtaccaccct gggcaacatg gtgaaacccc atcactacag aaaacacaaa aattagctgg |
| 14281 | gtatggtggc acatgctgtt agccccagct actcaggagg ctgaggtgca aggaggatag |
| 14341 | cctgagctca gcaggtggag gtttcattga gtggagagtg cgtgactgca ctccagcctg |
| 14401 | ggcgacagag cgagactctg tctcaaaaca aaacaaaaca aaacaaaaaa aacaaaaacc |
| 14461 | ttttgggctc atacaaaata tagaaaagca ataaagaata agatgtcatc catgatctca |
| 14521 | catcccaaac cctgtatctt ttaaaataaa ggggtgtttt tttttttttt agattagctt |
| 14581 | tatttgctca ccgtgaagat ggacctggaa tcccagcgga tattaaactt tttgatatat |
| 14641 | tttcacagca ggtggctaca gtgatacagg tttgtgtagc atttctccta agttctcaaa |
| 14701 | actttgaaac ttctctgcct tccttttaca attgtttaaa ataaattgtg tggttttcta |
| 14761 | aacattccag tctagacaag acatgccttc agaggatgtt gtatctttac aagtctctct |
| 14821 | gattaatctt gccatgaaat gttaccctga tcgtgtggac tatgttgata aagttctaga |
| 14881 | aacaacagtg gagatattca ataagctcaa ccttgaacag taagtcagtt acatttttgt |
| 14941 | aaaaatcctc aagatatttt ttgtcctaga tttgcttttc tttctcaatt gttttgtgaa |
| 15001 | ctgctggcat ttgtcttgtt ttaatcatgc attaagattg tcatgcttag cactactagg |
| 15061 | ggcagaaagt agtgaccaat tacttgtttt tttatattaa ggaaattgtg gtacctatgg |
| 15121 | accataggca gtcttcaggg accagtgtct ccaatttgga tccctttctg tgtgtcaggg |
| 15181 | gcatccaatc ttttggcttc cctgggctgc actggaagaa gcattttctt gggccacaca |
| 15241 | taaaatacac taacactaac aatagctgat gagcttaaaa aaaaaatccc aaaaaaactc |
| 15301 | ataatgtttt aagaaagttt acgaatttgt gttgggccgc attcaaagcc atcctgggct |
| 15361 | gcatgcggcc tgtgggcttt gggttggaca agcttgcatg tgactgagtt tgttcttaaa |
| 15421 | ctggtaagga aactttgtca ggcagtattt atttccataa gtggtgtttt cctacgaatc |
| 15481 | agcacatggt gaaaaatgag gggctatgta tatttaaggt gcagaattaa attggtttaa |
| 15541 | atatcttttc tattttgagc tttgattttg ataccttaaa ggaaatatca acagtactat |
| 15601 | ttccaacctg aagcctcctc agctgttctg tcctagactt atggcgtcct ctagtggcca |
| 15661 | ctatgggcag ctatgatcct gttaccttcc ccagcagttc ccttcctgcc ctgttcccca |
| 15721 | ctgctctggc ttgggtcaag ccaggcctgc ctcccgccaa catattcttc agaattttac |
| 15781 | ctcatgtaat cttcctcctt tctatctccc ttccagtggt ttacctgcat caagaaaatt |
| 15841 | tcttcttttt ttcctccctt tgtgttaccc ttgttctttt ggtcattttt ggttttgtgt |
| 15901 | gtgtgcaaac tgaaaacaag tccagatgtg gaatgataag tgtgagagaa aattaaatga |
| 19961 | tgtgccaggt gtggtggctt gcacctgtaa tcccagctat tcaggaggct gaattgggag |
| 16021 | aatcacttga gtccatgagt ttgagaacag cctgggcaac atagcgagac cccgtctcta |
| 16081 | ataaaaaata aaattaaaaa taaaaaaaat ttaaattaaa aaaactaaat gatgtatctg |
| 16141 | tgtctttctc cccaagtgaa ttttaaagta aaaatagaca aagtaattag aaataacaac |
| 16201 | ctctaaagag gttgtaataa atgccccaat atgcctcaat atctacagaa tgattttact |
| 16261 | aacaactacg taaaagtcag tcagcctgct tttccttaat caccaacatc tgatgcagaa |
| 16321 | gaaatagttt atgtgttttt ctgttgtgtc aaattgctgg ttttgcatgg agtttttttc |
| 16381 | ctatttattt tcatcatgaa tatacaatac ttgttggctg gcccctggga accaaactac |
| 16441 | cacttaaaat acttccctta gaaatgtcat caaattctag acagtcatct taactccagc |
| 16501 | tataccatct gttcatgagt tggaaactgt atctagtttt gtatcaacag aaaaataata |
| 16561 | gatgaatata tatttgtgtt tagataagca tttttatcct cctgaaagga ggttgttata |
| 16621 | gtcttctgtg gtggtatgat tcacttgacc catttccttt aatgtgtaat gaaaaatttc |
| 16681 | aaattcttat ggaacaaatg ctatttgtgt atatagaaag ttaattttat tcattaagac |
| 16741 | ttctgttttt ctttttgtag tattgctacc agtagtgcag tttcaaagga actcaccaga |
| 16801 | cttttgaaaa taccagttga cacttacaac aatattttaa cagtcttgaa attaaaacat |
| 16861 | tttcacccac tctttgagta ctttgactac gagtccagaa agagcatgag ttgttatgtg |
| 16921 | cttagtaatg ttctggatta taacacagaa attgtctctc aagaccaggt aagagaatac |
| 16981 | ctacgtgcta ttttagggaa acagtgttac aattttagac tttggaccta gatacctgag |
| 17041 | atgggagggg agggtaattc aatactaaat aaaatttaca agtaactttt tcattatata |
| 17101 | aattaaaaat tggagatgta taaagaatta taaaacattt ataattccac cagatagaga |
| 17161 | ataaccactg ttaattaaca tttggtgcat atctttccag acttttgtct gtatatgtgt |
| 17221 | gtatgacata catgtgtatc gactttctca ccaaaaaaag gaatatcttg ttgatactgt |
| 17281 | attgtaattt tataactgga aacacttttg ataatggctt tgtatgccaa tggtttcacc |
| 17341 | tcagtgggtt tcttgtgcct cgcatgttac aggtggattc cataatgaat ttggtatcca |
| 17401 | cgttgattca agatcagcca gatcaacctg tagaagaccc tgatccagaa gattttgctg |
| 17461 | atgagcagag ccttgtgggc cgcttcattc atctgctgcg ctctgaggac cctgaccagc |
| 17521 | agtacttggt atgagtttac ccttagtata tccctgtatc agctcctagt gaaatcacat |
| 17581 | gttcaagtgc ttaaaatggt ttaattcact ttctggtctt agatggtttt gaaggaattg |
| 17641 | caactgaatt aaagattcac ttgaacctgg gaggcggagg ttgtagtgag ccgagattgt |
| 17701 | gccactgcac tccagcctgg gcaacacagc gagactccat ctcgaaaaaa aaaaagattc |
| 17761 | atggcatcca tgggctttta ctttatatat aaacacataa ttgtttgtaa acttctggag |
| 17821 | catgtgagta acaattcagt tgctctgatt tcttttgaag actctctgag aattacaaaa |
| 17881 | aagtctgtct tcttttgctt gagtgccgat aattattcca tgttcatttt ttctgaacta |
| 17941 | tgtattgctt ataataaact ttataagaaa tacaattctt atatttaatt ttacttttcc |
| 18001 | aaatttgcaa gtataaatta tatttgtcat attgaaaatg tgagtttttg ttttttgatg |
| 18061 | aaagatttaa aaattcattt tgcctttttc ttaacttttt tttttctgat aaagaacaat |
| 18121 | cacatgaggt tctctcttta ttattagtcc acagggaatc attgtgaaat ggataaaaca |
| 18181 | tgttgcctga gtaggtgtat cagtgaccga tactagatag atagtttatt ttagtgaagg |
| 18241 | gttagcacag ttggctgctt aattattgtt tgggcaaagt agtttaacca ttcttggatg |
| 18301 | cataaggcta ttaggctgct atgatgaaaa agacatttgc ttgaggatgt cctgactgtc |
| 18361 | tcatcccttt ctgttgactt tcttcattgt agttgacaca cctgtacttc ataatcagtg |
| 18421 | tgaaataaga ggctgacttc tgttgatagt gtgatggtct ttgtcttggt ttagtgacaa |
| 18481 | acattccagg actgtggtat tgtgctctgt gagctatgtg atctgtacag agtgactgtc |
| 18541 | ttaagtattt taactgattg ccttatgttt ctgtgtgaga ttgcttgtat ctgtgtgttt |
| 18601 | tcattttcta ttgcctacca aatatagtag ttagaaacta ttccttccgg cggggcatgg |
| 18661 | tggctcgcat ctataatcct ggcactttga gaggctgagg tggatggatc acctgaggtc |
| 18721 | aggagttcag gatcagcatg gccaacatgg tgaaacccca tctctactaa aaatgcaaaa |
| 18781 | aattagctgg gcgtggtggt gggcgcctgt aatcctagct gctcaggaga ctgaggcagg |
| 18841 | agaatcactt gaacctggga ggtggaggtt gcagtgagtg gatatcatgc cattgcactc |
| 18901 | cagcctgggc aacaagagtg aaactccgtc tcaaaaaaaa ttatgccttc tgcatgtggc |
| 18961 | tgattggtta ttcccatgta tggagatctt taatgatagg gtcattagct ctgactgccc |
| 19021 | ctaggggaaa tgcattctct tattcatcta ccatatcagg aatttcacaa aacctgaatg |
| 19081 | ccattgtgtc acatatacta aaaatatttt ataaactctg tgtttttctt gtaatttttc |
| 19141 | tgaattggct atatgttgtg ccatttcaga aaaaaaaatc caagaaaaac acagaattca |
| 19201 | tggaatattt cacaagtagc tcttttaaag tatgttagca ttttccttga cttaaatggt |
| 19261 | cttaaaattt ttttgaatga ggaggtatga tgtaccagta atatgcatat agttgttgtg |
| 19321 | tatcatagta atagttaata ttactgagct tatgccttgt gctaagtagt ggtaagcctt |
| 19381 | cacatgtgtc acttgatctt cccaacaacc ctaggagttt atagaaactt gtggctaaga |
| 19441 | gaaggtaaat aatttgccca aggccacaca tgtaataagt attagcatct gcttttaaat |
| 19501 | gtgagtctct gagtatcttc acagccttct ttttttctct tttctttttt cttttttttt |
| 19561 | ttttttgaga tggaatcttg ctctgtcacc caggctggag tgcagtggca tgatcccagc |
| 19621 | ttactgcaac ctccatctcc tgggttcaag caattctcct gcctcagcct cctcagtagc |
| 19681 | tgagattacg ggtgtgcgcc accatgccca gctaattttt gtatttttag tagatacagg |
| 19741 | gtttccctat gttggccagg ctggtctcga actcctgacc tcaactgatc tgtccacctt |
| 19801 | cggcctccca aagtgctggg attacagaca tgagtcacca cacctggcca gagcctacat |
| 19861 | tctttatcag tgcagcatac tttgcacatg tgtgtatgaa aatatattta aatatatctt |
| 19921 | tgcttctaac tcgctacctt gggcaggtta tacaacctct ctgaaactca ggcttcccca |
| 19981 | tttgtgaaat ggaatagtat ctgtctctgg gttgttgtga caacttgagg agataagaaa |
| 20041 | tatgtaaatt gcctaccata aagtatggta cattgtatat attcacaaaa tgttagcaat |
| 20101 | gatgattaga gcccacattt atttcacaaa tgattaatca gagtttggaa attttttttt |
| 20161 | ctttaatgct tttgggtcag attttgaaca cagcacgaaa acattttgga gctggtggaa |
| 20221 | atcagcggat tcgcttcaca ctgccacctt tggtatttgc agcttaccag ctggcttttc |
| 20281 | gatataaaga gaattctaaa gtggtgagtt tacttttaag tatttaggta ctttttttcc |
| 20341 | tctttcatca ctctgagtgt gtgtgtgttt gttttatatt ataaaaaatt tcaaacgtac |
| 20401 | aaaaatagac agtggtataa taaaatccca ttttcgccaa ctctctattt gttacttatc |
| 20461 | ctgtgctaag tgttcctaac ggtgatggtg gtggatcaca tactgaggga tcacgtaaaa |
| 20521 | agcacttaga aatgcaagat taaagcagtg tgaatttatg ctgaaactct ttcctaagtt |
| 20581 | ctaattcagg ttagctttaa aacctaagga gagggcctag cattgcagtc gtttctctct |
| 20641 | aaaggcatat cattgaataa tatgagttgt gggcaacttt ttatgagctt ttttcttcct |
| 20701 | caaaatggaa ccatggcttg agtcttcaca gtgtagtttt gaagaaaata cctcaagctc |
| 20761 | acgtacctga aagttggaca ttcaggttaa tgttaaggaa caacctcagt aacttaattt |
| 20821 | tgtttgtttg tttgttttga gatagggtct cattctgtcg cccgggctgg agtacagtgg |
| 20881 | cgcagtctta gctcactgca acctccaact cctgggttca agcgattctt gtgtgtcagc |
| 20941 | ctcctaagta gctgggatca caggtgtgca ccaccatgcc cagctaattt ttgtattttt |
| 21001 | agtagagaca gggtcttgcc atgttgacca gttggtctcg aacttgtggc ctcaggtgat |
| 21061 | cctgctgcct cagcctctca aagtgctagg attgtaggtg tgaaccactg catctggcct |
| 21121 | cagtatggac ttgattttct cgtaatagag aaaaaagatg tatgcagtag acctaccagc |
| 21181 | atgaaacagc agcttttggc caatttttat taggccagct tatcattcac tctttaccag |
| 21241 | cgtttatgga taggaatttg tgaatataac aataaaaata gcaaccagcc tacattacaa |
| 21301 | agccatagta attaaagcag tatggtaata tggtactggc ataaaaacag acacatagac |
| 21361 | caatggaaca gaatagagag tctagaaata aacccacaca tatgcaataa actaatcttt |
| 21421 | gataaggaca ccaagaatac acaaagggga aaagaatggt ctcttcaata aatagtattg |
| 21481 | ggaaagttgg atatccacat gcaaaagaac gcatttggac tctcatctta tgccatatat |
| 21561 | aataatgaac tcaaaatgga ttaaagacct gaaaccataa agctcctaga agaaaacata |
| 21601 | gggaaaaacc tccttgtcat tggtcaatga ttttttggat atgaaaccaa aaacctatgc |
| 21661 | aactaaagca aaaataagtt taaaaataag caaaaaataa gtttaaaata agcttaaaat |
| 21721 | aagcaaaaat aagtttaaaa taagcaaaaa ataagcaaaa ataagtttaa taaactaaaa |
| 21781 | accttctgta caacaaagga aacaatcagc agagtgaaga gacaggcaat ggaatggggg |
| 21841 | agaatatttg caaactatac atctgaaaag tggtcaatat ctaaaatata tatggaatgc |
| 21901 | aactcaatag caagcaaatg aataacttga tttaaaaatg agcaaaggat ctgaatagac |
| 21961 | atttttccaa agaagacata caggtggcca actggtatat gaacagatgt tcaacatcat |
| 22021 | ttatcaggaa aatgtaaatc aaaaccacta tgagatgtca cctcacatct gtcagaataa |
| 22081 | ctgttatcaa aaaaacagaa aatcaagtgt tggcaaggat gtagagaaat gggaaccctg |
| 22141 | tttattattg gtgggaatat aaattagtat agccattatg gaaaacagta tggaggttcc |
| 22201 | tcaaaaaact gaaactagaa ctaccatgtg accctgcagt cccacatcta gttatgcatt |
| 22261 | caaaggaaag gaaatcagta tctcaaagag atatctgcac tcccatgttt attgcagcat |
| 22321 | tattcacaat ggctgagata tggaaacaac cttagtgtcc atcgatagat gagtaaagaa |
| 22381 | attgtgttgt gtatatatgt gtgtgtatat acgtatatat gtgtgtatat gtatgtacgc |
| 22441 | acatattctc tacatagtag aataatactc agctatagaa atgaagaaaa tcttgccatt |
| 22501 | tatgacaaca gggattaatc tggaggacat tgttctaagt gaaataagcc aaacacagaa |
| 22561 | aggcaaatac tatatgactt catttatatg tagaattgtt ttttaagttg aattcatcca |
| 22621 | gcctgggtaa tatagcaaga cccaatctct attaaaaaat aaaaaggcca ggtgtggggg |
| 22681 | ctcacgcctg taatcgcagc actttgggag gccgaagcag gcggttcacc tgaggtcggg |
| 22741 | agtttgagaa cagcctgacc aacatggaga aaccccgtct ctactgaaaa tacaaaatta |
| 22801 | gctgggcgtg gtggcgcatg cctgtagtcc cagctactcg ggagtctgag gcaggagaat |
| 22861 | cacttgaacc cgggacgcag aggttgtggt gagccgagat cgtgccattg cacttcagcc |
| 22921 | tgggcaacaa gagtgaaact ccgtctaaaa aaaaaaaaaa aaaattaaaa aattagccag |
| 22981 | gcgtggtggt acatgcctat agtcctagct actcaagagg ctagggcagg gctgggtgtg |
| 23041 | gtggctcaca cctgtcatcc tagcactttg ggaggccaag gtgggtggat catttgggat |
| 23101 | caggagtttg agaccagcct ggccaacatg gtgaaaccct gtctctacta aaaatacaaa |
| 23161 | aattagccag gtgtgagggg acatgcctgt aatcccagct acttgggaag ctgaggcagg |
| 23221 | agaatcactt gaacccggga ggcagaggtt gcagtaagct gagatcgcgc cactgcactc |
| 23281 | cagcctgggt gacaaagtga gactctgtct caaaaaaaag aggctagggc aggaggacag |
| 23341 | cttgagccca ggagttggag gctgcagtga gttatgattg tgcttttgcg ctccagcctg |
| 23401 | ggtgacagag ggagacccag tcactaaaaa atggttgaac ttgtgtaagc aaagactaga |
| 23461 | acagtagttg ccaaagaata caaactttca gttataagat aaaaaaattc tggggatcaa |
| 23521 | aacgatttag ggcaaataaa taaaagtaac tagcctttac ttatttacta gcatttctta |
| 23581 | ctgtgttgtc acccactgtg ccaaggtcta tgactgccac tgtcactttt tttttttttt |
| 23641 | ttgagtcaag gtctttgttg cccagactgg gatacagtgg tatgattacg gctcactgca |
| 23701 | gcttcgaact cttaggctca agcgatcctt ccatttcagc ctcctgagta gctgggactg |
| 23761 | caagcatgtg ccaccacact ggctaatttt ttattttttg tagagacaga gtctcaccat |
| 23821 | gttgcctagg ctggtctgaa actcttgggc tcaagcgatc ctcctgcctc cttggcctcc |
| 23881 | ctaagtgttg ggattacagg catgagccac catgcccagc ctgtcgccat cttttaaaaa |
| 23941 | tgaaaagaac tgattgcttt aacaagaaga aatttggata gtcaatcatg ataaaatatt |
| 24001 | taacctcgct tgtaattaca acagcgaacc ttttaagaaa tcaaattggc aaagagaaat |
| 24061 | gaaaaataag gttcagcaat ggcgatggtg tgatgaaaat tcattctcat ctaatgttgg |
| 24121 | cagtgtgaat tactataata cttctaggaa gttggcggtg tgtaagtagg gtgttaaaat |
| 24181 | attcaataat tttacttcca agtggaattc caagaattta tactaaggga ataattaggg |
| 24241 | tctcaataaa gcttagtgta tatagaacat tcattgtaat attacagatt atgtctaaaa |
| 24301 | gggaatagtt caataaatta tgccatagcc agtctccata atattttcta gtcattaaaa |
| 24361 | tgatttcgaa ttagtatcgg gaagattgtt aggacaaaat aggaaaaatt agagctgggt |
| 24421 | gcagtggctc acgcctgtaa tcctagcact ttgggaggct gaggcaggcg gatcacctga |
| 24481 | ggtcgggagt ttgagaccag cctgaccaat atggagaaac cccgtctcta ctaaaaatac |
| 24541 | aaaattagct gggtgtggtg gcgcatgcct gtaatcccag ctattcggta ggctgaggca |
| 24601 | ggagaatcac ttgaacctgg gaggcggagg ttgtggcgag ctgagatcat gccattgcac |
| 24661 | tccagcctgg gcaacaagag ggaaactact tctcaaaaaa aaaaaaagaa aagaaaagaa |
| 24721 | aaattagata caaattactt gaagtgtgaa tcgattttaa ctctcaagaa aataaggtct |
| 24781 | agatgcagtg gctcacgcca gtaatcctag cactttggga ggctgagatg ggtggatcag |
| 24841 | ttgaggtcag cagttcaaga ctagcctggc cgataaggtg aaaccccttc tctactaaaa |
| 24901 | atacaaaaaa tagcagggcg tggtggcgcg cttgtaatcc cagctactca ggaggctgag |
| 24961 | gcagaagaat ggtttgaacc caagaggcag aggtggcagt gagccgagat cgcaccaaag |
| 25021 | agaaaaaaga aaaccacaca caaaaatgcc agttatatta cagttacata gaaaaaaaga |
| 25081 | aaggaagaca tttagcatcc gaatgttacc agtgattatc cgtgggtggt agatttaggg |
| 25141 | atgatgtgtg gatgattttg tgtatttttc taattttctc caatttggga atgtaactta |
| 25201 | caaatcagaa aaaacaatta tcagccaggt gtgatggctc atgcctgtaa tcccaacact |
| 25261 | ttgggaggct gaggcgggag gtttgctcgg ggccagtagt tcaagatcag cctgggcaac |
| 25321 | agaatgagac cctgtctcta caaaaaaaaa aaaaaaaaaa aaaaaaaaaa ttagccaggt |
| 25381 | gtggtgatgc aagcctgtag tcctagctat tcgggagtct gaggtgggag catcacttga |
| 25441 | acccaggagt tcaaggctgc aatgagctgt gatcacacca ctgcactcca gctgggtaac |
| 25501 | agagctgttg aaaaaaaaaa aaggaaagaa aaaacaggtt gagtgcagtg gctcacgcct |
| 25561 | gtgatcccag cactttggga ggctgaggcg ggcagatcac ttgaggtcag gagttaccag |
| 25621 | cctggccaac ttggtgaaac cccgccccac ttggtgaaac cccgccccta ctaaaaatac |
| 25681 | aaaattagct gggtgtggtg gtgggcacct gtaatcccag ctactcggga ggctgaggca |
| 25741 | ggagaatcac ttgaatccag gagacggagg ttgcagtgag ccaagattgt gccactgcac |
| 25801 | tccagcctgg gcaacaagag caaaactctg tctcaaaaag aaaaaacaaa taccaaatac |
| 25861 | attaacattg caaaggcaat ttaacctcaa atgatgtttt gagaagacat cctgcttgat |
| 25921 | ttacttgttt gccctataac tgaaacagag aaggaaaatg acaggaaaac tgtgcacaca |
| 25981 | acttacagta ttttgttcta ttaaaatgga tatcctggaa caagttaatt ttgaatttaa |
| 26041 | ggtaacttaa aatgtttttt cttgttttag gatgacaaat gggaaaagaa atgccagaag |
| 26101 | attttttcat ttgcccacca gactatcagt gctttgatca aagcagagct ggcagaattg |
| 26161 | cccttaagac tttttcttca aggagcacta gctgctgggg aaattggttt tgaaaatcat |
| 26221 | gagacagtcg catatgaatt catgtcccag gtgatgatct gttctttctg cgttgtcatg |
| 26281 | tcagctctgc tgggttcagt tgcttgtttg caggcatggt ggtaatgcac atgaatttac |
| 26341 | tcttctttta ctgaatgtgt aactaccacc ttcccaccat catggaacct gttaatatta |
| 26401 | ttgttgtaat tgactggtgt tgatcatttg ctgatgaaat ctaagatttc caagtgggtc |
| 26461 | atggtaaaaa tgtttcatgg aacataaaat tcgggaaatg cactcaattc ccaaaatcca |
| 26521 | gtttgggaac cctgggttaa acaaagttga aagaagtttc tttattgcaa ctttttagcg |
| 26581 | tttttaccat ctcagttgtg tcctgtggct ctcaagagag ggtgcagcat gttctgatat |
| 26641 | gaaggctgca gaagtctcac aggatggagg tttggtgaca agtactttgg aaaatgctca |
| 26701 | actagaggat ggttggtcct tgaaagtcct ttctgcttta tgttcactag gcattttctc |
| 26761 | tgtatgaaga tgaaatcagc gattccaaag cacagctagc tgccatcacc ttgatcattg |
| 26821 | gcacttttga aaggatgaag tgcttcagtc aagagaatca tgaacctctg aggactcagt |
| 26881 | gtgcccttgc tgcatccaaa cttctaaaga aacctgatca gggccgagct gtgagcacct |
| 26941 | gtgcacatct cttctggtct ggcagaaaca cggacaaaaa tggggaggag gtaaggtcat |
| 27001 | tcctgactgc atgatagcag acaggatcca taacagggat cagttgtcat ggccttgtgt |
| 27061 | tctggaggtg aaacatttgg ggtgcttgga aatctgatga acaaaattgc tttgttttgt |
| 27121 | taaaaaagag agtctcatcc tgtagtgaag cctctgcttt gaggatattg taacatagca |
| 27181 | agttcaaacc actacctgtt tttaaaaaaa tacagctgta tacttcaaaa caagaagaag |
| 27241 | gagaatgaaa aggatttaaa tttgttatgt ccctttaaaa cacgaaagag ccacggtagt |
| 27301 | gttgtgtttc tttgtatgaa aacgagatgt ttcattaatc tcttcactgt ccccctgccc |
| 27361 | ttttatttta gcttcacgga ggcaagaggg taatggagtg cctaaaaaaa gctctaaaaa |
| 27421 | tagcaaatca gtgcatggac ccctctctac aagtgcagct ttttatagaa attctgaaca |
| 27481 | gatatatcta tttttatgaa aaggaaaatg atgcggtaag tgaattagta aagtgttgtt |
| 27541 | aataaactaa tattttccct tcctactctt aggagatttg atatgtacaa aagtttatca |
| 27601 | ttctgatact ttaatcactg ttcatttgaa aaatgtaaaa taatttacag atgtcaaata |
| 27661 | ataggctaat ttgtcataat gttctagttt aagataattc ctaggctggg cgtggtggct |
| 27721 | catgcctgta atcccagcac tttgggaggc tgaggcaggc agatcacctg aggtcaggag |
| 27781 | tttgaaacca gcctggccaa cattgtgaaa ccccatctct actaaaaata caaaaattag |
| 27841 | ctaggcgtgg tggcaggcgc ctgtaatccc agctacttgg aagcctaagg caggagaatc |
| 27901 | gcttgaacct gggaggtgga ggctgcagtg agccaagact gtgccattgc actcctgcct |
| 27961 | gggcaacaag agtgaaactc cgtctcaaaa ataataataa taataattcc taaacgcagt |
| 28021 | atccttttag caatacagtt ttggtcaaga tttgtaagtt aaataaaatt ttgcttgttt |
| 28081 | ttcttttttt gacagagtct ggctctgtca cccaggctgg agtgcagtgg caatctcagc |
| 28141 | tcattgcaac ctctgcctcc caggttcaag caattctcat gcctcagcct cctgaatagc |
| 28201 | tggtattata ggcgcccggc accacgccca gctaattttt gtattattac tagagatggg |
| 28261 | gttccaccat gttggccagg ctggtctcaa aactcctgac ttcaagtgat ctgcccacct |
| 28321 | cagcctccca aagtgctggg agtacaggga tgagccactg agccgagcca attttgcttg |
| 28381 | ttttaaaggg ttgttttttt tttttttttt ttgatagtca gtaattgttc aaactaggaa |
| 28441 | ttgtatcccc atctttcttt tttcataatt actcaggtaa ttgatgagtg taacagaagc |
| 28501 | tcctcaaaac agttttatta aattgccttt cattttttgt ggtacgtgct tgatcatgaa |
| 28561 | tttgtacata ttcttttgta ggtaacaatt caggttctaa accagcttat ccaaaagatt |
| 28621 | cgagaagacc tcccgaatct tgaatccagt gaagaaacag agcagattaa caaacatttt |
| 28681 | cataacacac tggagcattt gcgcttgcgg cgggaatcac cagaatccga ggggccaatt |
| 28741 | tatgaaggtc tcatccttta aaaaggaaat agctcaccat actcctttcc atgtacatcc |
| 28801 | agtgagggtt ttattacgct aggtttccct tccatagatt gtgcctttca gaaatgctga |
| 28861 | ggtaggtttc ccatttctta cctgtgatgt gttttaccca gcacctccgg acactcacct |
| 38921 | tcaggacctt aataaaatta ttcacttggt aagtgttcaa gtctttctga tcaccccaag |
| 28981 | tagcatgact gatctgcaat ttaaaattcc tgtgatctgt aaaaaaaaaa aaaaaaaaaa |
| 29041 | aaacaaaacc cacaagcact tatcttggct actaatgaag ctctcctttt ttttgtttgt |
| 29101 | ttgtttgctt cattgttgat tgtgtatttt cttcattcct ggggagtact aacccaaaag |
| 29161 | cgtctgtctc ttgttttcta gtccagtttg agattaattt agaagaaagg aatactgtat |
| 29221 | gtgaaattca tcttgggctt tcccctaaat tgcaagataa ggccatgtgt aagattttcc |
| 29281 | ctaaaactag aatatattaa tgcatgtttg agaattttaa agcaccatgg tcaaaaccag |
| 29341 | aagctatatt ttgcatattt ggactcagcc atccattaag aacccatgtt gtcctctgga |
| 29401 | catatttatc aatataattg ggttttaagt agtataaaag aaaacttgtg atctatataa |
| 29461 | tttatgtatc accttcattg taaatttagc aggaaatgca tcacaattat gatttttttt |
| 29521 | tttgcaccag tgaaacaata aagatgctat taacaa |

For example, the nucleotide sequence corresponding to the mRNA of the human VPS35 is depicted in SEQ ID NO: 13 (3298 bp), wherein the underscored bolded “ATG” denotes the beginning of the open reading frame. Sequence information related to LRRK2 VPS35 is accessible in public databases by GenBank Accession number NM_018206.4 (nucleotide).

SEQ ID NO: 13:

| | |
|---|---|
| 1 | gctagagagg gcggggcttg gaggggccgc agcgtcacat gaccgcggga ggctacgcgc |
| 61 | ggggcgggtg ctgcttgctg caggctctgg ggagtcgcca tgcctacaac acagcagtcc |
| 121 | cctcaggatg agcaggaaaa gctcttggat gaagccatac aggctgtgaa ggtccagtca |
| 181 | ttccaaatga agagatgcct ggacaaaaac aagcttatgg atgctctaaa acatgcttct |
| 241 | aatatgcttg gtgaactccg gacttctatg ttatcaccaa agagttacta tgaactttat |
| 301 | atggccattt ctgatgaact gcactacttg gaggtctacc tgacagatga gtttgctaaa |
| 361 | ggaaggaaag tggcagatct ctacgaactt gtacagtatg ctggaaacat tatcccaagg |
| 421 | ctttaccttt tgatcacagt tggagttgta tatgtcaagt catttcctca gtccaggaag |
| 481 | gatattttga aagatttggt agaaatgtgc cgtggtgtgc aacatccctt gaggggtctg |
| 541 | tttcttcgaa attaccttct tcagtgtacc agaaatatct tacctgatga aggagagcca |
| 601 | acagatgaag aaacaactgg tgacatcagt gattccatgg attttgtact gctcaacttt |
| 661 | gcagaaatga acaagctctg ggtgcgaatg cagcatcagg gacatagccg agatagagaa |
| 721 | aaaagagaac gagaaagaca agaactgaga attttagtgg gaacaaattt ggtgcgcctc |
| 781 | agtcagttgg aaggtgtaaa tgtggaacgt tacaaacaga ttgttttgac tggcatattg |
| 841 | gagcaagttg taaactgtag ggatgctttg gctcaagaat atctcatgga gtgtattatt |
| 901 | caggttttcc ctgatgaatt tcacctccag actttgaatc cttttcttcg ggcctgtgct |
| 961 | gagttacacc agaatgtaaa tgtgaagaac ataatcattg ctttaattga tagattagct |
| 1021 | ttatttgctc accgtgaaga tggacctgga atcccagcgg atattaaact ttttgatata |
| 1081 | ttttcacagc aggtggctac agtgatacag tctagacaag acatgccttc agaggatgtt |
| 1141 | gtatctttac aagtctctct gattaatctt gccatgaaat gttaccctga tcgtgtggac |
| 1201 | tatgttgata aagttctaga aacaacagtg gagatattca ataagctcaa ccttgaacat |
| 1261 | attgctacca gtagtgcagt ttcaaaggaa ctcaccagac ttttgaaaat accagttgac |
| 1321 | acttacaaca atattttaac agtcttgaaa ttaaaacatt ttcacccact ctttgagtac |
| 1381 | tttgactacg agtccagaaa gagcatgagt tgttatgtgc ttagtaatgt tctggattat |
| 1441 | aacacagaaa ttgtctctca agaccaggtg gattccataa tgaatttggt atccacgttg |
| 1501 | attcaagatc agccagatca acctgtagaa gaccctgatc cagaagattt tgctgatgag |
| 1561 | cagagccttg tgggccgctt cattcatctg ctgcgctctg aggaccctga ccagcagtac |
| 1621 | ttgattttga acacagcacg aaaacatttt ggagctggtg gaaatcagcg atttcgcttc |
| 1681 | acactgccac ctttggtatt tgcagcttac cagctggctt ttcgatataa agagaattct |
| 1741 | aaagtggatg acaaatggga aaagaaatgc cagaagattt tttcatttgc ccaccagact |
| 1801 | atcagtgctt tgatcaaagc agagctggca gaattgccct taagactttt tcttcaagga |
| 1861 | gcactagctg ctggggaaat tggttttgaa aatcatgaga cagtcgcata tgaattcatg |
| 1921 | tcccaggcat tttctctgta tgaagatgaa atcagcgatt ccaaagcaca gctagctgcc |
| 1981 | atcaccttga tcattggcac ttttgaaagg atgaagtgct tcagtgaaga gaatcatgaa |
| 2041 | cctctgagga ctcagtgtgc ccttgctgca tccaaacttc taaagaaacc tgatcagggc |
| 2101 | cgagctgtga gcacctgtgc acatctcttc tggtctggca gaaacacgga caaaaatggg |
| 2161 | gaggagcttc acggaggcaa gagggtaatg gagtgcctaa aaaaagctct aaaaatagca |
| 2221 | aatcagtgca tggacccctc tctacaagtg cagcttttta tagaaattct gaacagatat |
| 2281 | atctattttt atgaaaagga aaatgatgcg gtaacaattc aggttttaaa ccagcttatc |
| 2341 | caaaagattc gagaagacct cccgaatctt gaatccagtg aagaaacaga gcagattaac |
| 2401 | aaacattttc ataacacact ggagcatttg cgcttgcggc gggaatcacc agaatccgag |
| 2461 | gggccaattt atgaaggtct catcctttaa aaaggaaata gctcaccata ctcctttcca |
| 2521 | tgtacatcca gtgagggttt tattacgcta ggtttccctt ccatagattg tgcctttcag |
| 2581 | aaatgctgag gtaggtttcc catttcttac ctgtgatgtg ttttacccag cacctccgga |
| 2641 | cactcacctt caggacctta ataaaattat tcacttggta agtgttcaag tctttctgat |
| 2701 | caccccaagt agcatgactg atctgcaatt taaaattcct gtgatctgta aaaaaaaaaa |
| 2761 | aaaaaaaaaa aacaaaaccc acaagcactt atcttggcta ctaatgaagc tctccttttt |
| 2821 | tttgtttgtt tgtttgcttc attgttgatt gtgtattttc ttcattcctg gggagtacta |
| 2881 | acccaaaagc gtctgtctct tgttttctag tccagtttga gattaattta gaagaaagga |
| 2941 | atactgtatg tgaaattcat cttgggcttt cccctaaatt gcaagataag gccatgtgta |
| 3001 | agattttccc taaaactaga atatattaat gcatgtttga gaattttaaa gcaccatggt |
| 3061 | caaaaccaga agctatattt tgcatatttg gactcagcca tccattaaga acccatgttg |
| 3121 | tcctctggac atatttatca atataattgg gttttaaata gtataaaaga aaacttgtga |
| 3181 | tctatataat ttatgtatca ccttcattgt aaatttagca ggaaatgcat cacaattatg |
| 3241 | attttttttt ttgcaccagt gaaacaataa agatgctatt aacaaaaaaa aaaaaaaa |

For example, the polypeptide sequence corresponding to human VPS35 is encoded by the nucleic acid sequence of SEQ ID NO: 13 and is depicted in SEQ ID NO: 14 (796aa). Sequence information related to VPS35 is accessible in public databases by GenBank Accession numbers NP_060676.2 (protein).

| | |
|---|---|
| 1 | MPTTQQSPQD EQFKLLDEAI QAVYVQSFQM KRCLDKNKLM DALKHASNML GELRTSMLSP |
| 61 | KSYYELYMAI SDELHYLEVY LTDEFAKGRK VADLYELVQY AGNIIPRLYL LITVGVVYVK |
| 121 | SFPQSRKDIL KDLVEMCRGV QHPLRGLFLR NYLLQCTRNI LPDEGEPTDE ETTGDISDSM |
| 181 | DFVLLNFAEM NKLWVRMQHQ GHSRDREKRE RERQELRILV GTNLVRLSQL EGVNVERYKQ |
| 241 | IVLTGILEQV VNCRDALAQE YLMECIIQVF PDEFHLQTLN PFLRACAELH QNVNVKNIII |
| 301 | ALIDRLALFA HREDGPGIPA DIKLFDIFSQ QVATVIQSRQ DMPSEDVVSL QVSLINLAMK |
| 361 | CYPDRVDYVD KVLETTVEIF NKLNLEHIAT SSAVSKELTR LLKIPVDTYN NILTVLKLKH |
| 421 | FHPLFEYFDY ESRKSMSCYV LSNVLDYNTE IVSQDQVDSI MNLVSTLIQD QPDQPVEDPD |
| 481 | PEDFADEQSL VGRFIHLLRS EDPDQQYLIL NTARKHFGAG GNQRIRFTLP PLVFAAYQLA |
| 541 | FRYKENSKVD DKWEKKCQKI FSFAHQTISA LIKAELAELP LRLFLQGALA AGEIGFENHE |
| 601 | TVAYEFMSQA FSLYEDEISD SKAQLAAITL IIGTFERMKC FSEENHEPLR TQCALAASKL |
| 661 | LKKPDQGRAV STCAHLFWSG RNTDKNGEEL HGGKRVMECL KKALKIANQC MDPSLQVQLF |
| 721 | IEILNRYIYF YEKENDAVTI QVLNQLIQKI REDLPNLESS EETEQINKHF HNTLEHLRLR |
| 781 | RESPESEGPI YEGLIL |

For example, the polypeptide sequence corresponding to human RAB7L1 (isoform 1) has a mutation wherein the amino acid at position 67 is a lysine (L) instead of a glutamine (Q) and is depicted in SEQ ID NO: 26 (203aa).

SEQ ID NO: 26:

| | |
|---|---|
| 1 | MGSRDHLFKV LVVGDAAVGK TSLVQRYSQD SFSKHYKSTV GVDFALKVLQ WSDYEIVRLQ |
| 61 | LWDIAGLERF TSMTRLYYRD ASACVIMFDV TNATTFSNSQ RWKQDLDSKL TLPNGEPVPC |
| 121 | LLLANKCDLS PWAVSRDQID RFSKENGFTG WTETSVKENK NINEAMRVLI EKMMRNSTED |
| 181 | IMSLSTQGDY INLQTKSSSW SCC |

For example, the polypeptide sequence corresponding to human LRRK2 has a mutation wherein the amino acid at position 2019 is a serine (S) instead of a glycine (G) and is depicted in SEQ ID NO: 27 (2527aa).

SEQ ID NO: 27:

| | |
|---|---|
| 1 | MASGSCQGCE EDEETLKKLI VRLNNVQEGK QIETLVQILE DLLVFTYSEH ASKLFQGKNI |
| 61 | HVPLLIVLDS YMRVASVQQV GWSLLCKLIE VCPGTMQSLM GPQDVGNDWE VLGVHQLILK |
| 121 | MLTVHNASVN LSVIGLKTLD LLLTSGKITL LILDEESDIF MLIFDAMHSF PANDEVQKLG |
| 181 | CKALHVLFER VSEEQLTEFV ENKDYMILLS ALTNFKDEEE IVLHVLHCLH SLAIPCNNVE |
| 241 | VLMSGNVRCY NIVVEAMKAF PMSERIQEVS CCLLHRLTLG NFFNILVLNE VHEFVVKAVQ |
| 301 | QYPENAALQI SALSCLALLT ETIFLNQDLE EKNENQENDD EGEEDKLFWL EACYKALTWH |
| 361 | RKNKHVQEAA CWALNNLLMY QNSLHEKIGD EDGHFPAHRE VMLSMLMHSS SKEVFQASAN |
| 421 | ALSTLLEQNV NFRKILLSKG IHLNVLELMQ KHIHSPEVAE SGCKMLNHLF EGSNTSLDIM |
| 481 | AAVVPKILTV MKRHETSLPV QLEALRAILH FIVPGMPEES REDTEFHHKL NMVKKQCFKN |
| 541 | DIHKLVLAAL NRFIGNPGIQ KCGLKVISSI VHFPDALEML SLEGAMDSVL HTLQMYPDDQ |
| 601 | EIQCLGLSLI GYLITKKNVF IGTGHLLAKI LVSSLYRFKD VAEIQTKGFQ TILAILKLSA |
| 661 | SFSKLLVHHS FDLVIFHQMS SNIMEQKDQQ FLNLCCKCFA KVAMDDYLKN VMLERACDQN |
| 721 | NSIMVECLLL LGADANQAKE GSSLICQVCE KESSPKLVEL LLNSGSREQD VRKALTISIG |
| 781 | KGDSQIISLL LRRLALDVAN NSICLGGFCI GKVEPSWLGP LFPDKTSNLR KQTNIASTLA |
| 841 | RMVIRYQMKS AVEEGTASGS DGNFSEDVLS KFDEWTFIPD SSMDSVFAQS DDLDSEGSEG |
| 901 | SFLVKKKSNS ISVGEFYRDA VLQRCSPNLQ RHSNSLGPIF DHEDLLKRKR KILSSDDSLR |
| 961 | SSKLQSHMRH SDSISSLASE REYITSLDLS ANELRDIDAL SQKCCISVHL EHLEKLELHQ |
| 1021 | NALTSFPQQL CETLKSLTHL DLHSNKFTSF PSYLLKMSCI ANLDVSRNDI GPSVVLDPTV |
| 1081 | KCPTLKQFNL SYNQLSFVPE NLIDVVEKLE QLILEGNKIS GICSPLRLKE LKILNLSKNH |
| 1141 | ISSLSENFLE ACPKVESFSA RMNFLAAMPF LPPSMTILKL SQNKFSCIPE AILNLPHLRS |
| 1201 | LDMSSNDIQY LPGPAHWKSL NLRELLFSHN QISILDLSEK AYLWSRVEKL HLSHNKLKEI |
| 1261 | PPEIGCLENL TSLDVSYNLE LRSFPNEMGK LSKIWDLPLD ELHLNFDFKH IGCKARDIIR |
| 1321 | FLQQRLKKAV PYNRMKLMIV GNTGSGKTTL LQQLMKTKKS DLGMQSATVG IDVKDWPIQI |
| 1381 | RDKRKRDLVL NVWDFAGREE FYSTHPHFMT QRALYLAVYD LSKGQAEVDA MKPWLFNIKA |
| 1441 | RASSSPVILV GTHLDVSDEK QRKACMSKIT KELLNKRGFP AIRDYHFVNA TEESDALAKL |
| 1501 | RKTIINESLN FYIRDQLVVG QLIPDCYVEL EKIILSERKN VPIEFPVIDR KRLLQLVREN |
| 1561 | QLQLDENELP HAVHFLNESG VLLHPQDPAL QLSDLYFVEP KWLCKIMAQI LTVKVEGCPK |
| 1621 | HPKGIISRRD VEKFLSKKRK FPKNYMSQYF KLLEKFQIAL PIGEEYLLVP SSLSDHRPVI |
| 1681 | ELPHCENSEI IIRLYEMPYF PMGFWSRLIN RLLEISPYML SGREPALRPN RMYWRQGIYL |
| 1741 | NWSPEAYCLV GSEVLDNHPE SFLKITVPSC RKGCILLGQV VDHIDSLMEE WFPGLLEIDI |
| 1801 | CGEGETLLKK WALYSFNDGE EHQKILLDDL MKKAEEGDLL VNPDQPRLTI PISQIAPDLI |
| 1861 | LADLPRNIML NNDELEFEQA PEFLLGDGSF GSVYRAAYEG EEVAVKIFNK HTSLRLLRQE |
| 1921 | LVVLCHLHHP SLISLLAAGI RPRMLVMELA SKGSLDRLLQ QDKASLTRTL QHRIALHVAD |
| 1981 | GLRYLHSAMI IYRDLKPHNV LLFTLYPNAA IIAKIADYSI AQYCCRMGIK TSEGTPGFRA |
| 2041 | PEVARGNVIY NQQADVYSFG LLLYDILTTG GRIVEGLKFP NEFDELEIQG KLPDPVKEYG |
| 2101 | CAPWPMVEKL IKQCLKENPQ ERPTSAQVFD ILNSAELVCL TRRILLPKNV IVECMVATHH |
| 2161 | NSRNASIWLG CGHTDRGQLS FLDLNTEGYT SEEVADSRIL CLALVHLPVE KESWIVSGTQ |
| 2221 | SGTLLVINTE DGKKRHTLEK MTDSVTCLYC NSFSKQSKQK NFLLVGTADG KLAIFEDKTV |
| 2281 | KLKGAAPLKI LNIGNVSTPL MCLSESTNST ERNVMWGGCG TKIFSFSNDF TIQKLIETRT |
| 2341 | SQLFSYAAFS DSNIITVVVD TALYIAKQNS PVVEVWDKKT EKLCGLIDCV HFLREVMVKE |
| 2401 | NKESKHKMSY SGRVKTLCLQ KNTALWIGTG GGHILLLDLS TRRLIRVIYN FCNSVRVMMT |
| 2461 | AQLGSLKNVM LVLGYNRKNT EGTQKQKEIQ SCLTVWDINL PHEVQNLEKH IEVRKELAEK |
| 2521 | MRRTSVE |

For example, the polypeptide sequence corresponding to human LRRK2 has a mutation wherein the amino acid at position 1441 is a cysteine (C) instead of an arginine (R) and is depicted in SEQ ID NO: 28 (2527aa).

SEQ ID NO: 28:

| | |
|---|---|
| 1 | MASGSCQGCE EDEETLKKLI VRLNNVQEGK QIETLVQILE DLLVFTYSEH ASKLFQGKNI |
| 61 | HVPLLIVLDS YMRVASVQQV GWSLLCKLIE VCPGTMQSLM GPQDVGNDWE VLGVHQLILK |
| 121 | MLTVHNASVN LSVIGLKTLD LLLTSGKITL LILDEESDIF MLIFDAMHSF PANDEVOKLG |
| 181 | CKALHVLFER VSEEQLTEFV ENKDYMILLS ALTNFKDEEE IVLHVLHCLH SLAIPCNNVE |
| 241 | VLMSGNVRCY NIVVEAMKAF PMSERIQEVS CCLLHRLTLG NFFNILVLNE VHEFVVKAVQ |
| 301 | QYPENAALQI SALSCLALLT ETIFLNQDLE EKNENQENDD EGEEDKLFWL EACYKALTWH |
| 361 | RKNKHVQEAA CWALNNLLMY QNSLHEKIGD EDGHFPAHRE VMLSMLMHSS SKEVFQASAN |
| 421 | ALSTLLEQNV NFRKILLSKG IHLNVLELMQ KHIHSPEVAE SGCKMLNHLF EGSNTSLDIM |
| 481 | AAVVPKILTV MKRHETSLPV QLEALRAILH FIVPGMPEES REDTEFHHKL NMVKKQCFKN |
| 541 | DIHKLVLAAL NRFIGNPGIQ KCGLKVISSI VHFPDALEML SLEGAMDSVL HTLQMYPDDQ |
| 601 | EIQCLGLSLI GYLITKKNVF IGTGHLLAKI LVSSLYRFKD VAEIQTKGFQ TILAILKLSA |
| 661 | SFSKLLVHHS FDLVIFHQMS SNIMEQKDQQ FLNLCCKCFA KVAMDDYLKN VMLERACDQN |
| 721 | NSIMVECLLL LGADANQAKE GSSLICQVCE KESSPKLVEL LLNSGSREQD VRKALTISIG |
| 781 | KGDSQIISLL LRRLALDVAN NSICLGGFCI GKVEPSWLGP LFPDKTSNLR KQTNIASTLA |
| 841 | RMVIRYQMKS AVEEGTASGS DGNFSEDVLS KFDEWTFIPD SSMDSVFAQS DDLDSEGSEG |
| 901 | SFLVKKKSNS ISVGEFYRDA VLQRCSPNLQ RHSNSLGPIF DHEDLLKRKR KILSSDDSLR |
| 961 | SSKLQSHMRH SDSISSLASE REYITSLDLS ANELRDIDAL SQKCCISVHL EHLEKLELHQ |
| 1021 | NALTSFPQQL CETIKSLTHL DLHSNKFTSF PSYLLKMSCI ANLDVSRNDI GPSVVLDPTV |
| 1081 | KCPTLKQFNL SYNQLSFVPE NLTDVVEKLE QLILEGNKIS GICSPLRLKE LKILNLSKNH |
| 1141 | ISSLSENFLE ACPKVESFSA RMNFLAAMPF LPPSMTILKL SQNKFSCIPE AILNLPHLRS |
| 1201 | LDMSSNDIQY LPGPAHWKSL NLRELLFSHN QISILDLSEK AYLWSRVEKL HLSHNKLKEI |
| 1261 | PPEIGCLENL TSLDVSYNLE LRSFPNEMGK LSKIWDLPLD ELHLNFDFKH IGCKAKDIIR |
| 1321 | FLQQRLKKAV PYNRMKLMIV GNTGSGKTTL LQQLMKTKKS DLGMQSATVG IDVKDWPIQI |
| 1381 | RDKRKRDLVL NVWDFAGREE FYSTHPHFMT QRALYLAVYD LSKGQAEVDA MKPWLFNIKA |
| 1441 | CASSSPVILV GTHLDVSDEK QRKACMSKIT KELLNKRGFP AIRDYHFVNA TEESDALAKL |
| 1501 | RKTIINESLN FKIRDQLVVG QLIPDCYVEL EKIILSERKN VPIEFPVIDR KRLLQLVREN |
| 1561 | QLQLDENELP HAVHFLNESG VLLHFQDPAL QLSDLYFVEP KWLCKIMAQI LTVKVEGCPK |
| 1621 | HPKGIISRRD VEKFLSKKRK FPKNYMSQYF KLLEKFQIAL PIGEEYLLVP SSLSDHRPVI |
| 1681 | ELPHCENSEI IIRLYEMPYF PMGFWSRLIN RLLEISPYML SGRERALRPN RMYWRQGIYL |
| 1741 | NWSPEAYCLV GSEVLDNHPE SFLKITVPSC RKGCILLGQV VDHIDSLMEE WFPGLLEIDI |
| 1801 | CGEGETLLKK WALYSFNDGE EHQKILLDDL MKKAEEGDLL VNPDQPRLTI PISQIAPDLI |
| 1861 | LADLPRNIML NNDELEFEQA PEFLIGDGSF GSVYRAAYEG EEVAVKIFNK HTSLRLLRQE |
| 1921 | LVVLCHLHHP SLISLLAAGI RPRMLVMELA SKGSLDRLLQ QDKASLTRTL QHRIALHVAD |
| 1981 | GLRYLHSAMI IYRDLKPHNV LLFTLYPNAA IIAKIADYGI AQYCCRMGIK TSEGTPGFRA |
| 2041 | PEVARGNVIY NQQADVYSFG LLLYDILTTG GRIVEGLKFP NEFDELEIQG KLPDPVKEYG |
| 2101 | CAPWPMVEKL IKQCLKENPO ERPTSAQVFD ILNSAELVCL TRRILLPKNV IVECMVATHH |
| 2161 | NSRNASIWLG CGHTDRGQLS FLDLNTEGYT SEEVADSRIL CLALVHLPVE KESWIVSGTQ |
| 2221 | SGTLLVINTE DGKKRHTLEK MTDSVTCLYC NSFSKQSKQK NFLLVGYADG KLAIFEDKTV |
| 2281 | KLKGAAPLKI LNIGNVSTPL MCLSESTNST ERNVMWGGCG TKIFSFSNDF TIQKLIETRT |
| 2341 | SQLFSYAAFS DSNIITVVVD TALYIAKQNS PVVEVWDKKT EKLCGLIDCV HFLREVMVKE |
| 2401 | NKESKHKMSY SGRVKTLCLQ KNTALWIGTG GGHILLLDLS TRRLIRVIYN FCNSVRVMMT |
| 2461 | AQLGSLKNVM LVLGYNRKNT EGTQKQKEIQ SCLTVWDINL PHEVQNLEKH IEVRKELAEK |
| 2521 | MRRTSVE |

A RAB7L1, a LRRK2, or a VPS35 molecule can also encompass ortholog genes, which are genes conserved among different biological species such as humans, dogs, cats, mice, and rats, that encode proteins (for example, homologs (including splice variants), mutants, and derivatives) having biologically equivalent functions as the human-derived protein. Orthologs of a RAB7L1, a LRRK2, or a VPS35 protein include any mammalian ortholog inclusive of the ortholog in humans and other primates, experimental mammals (such as mice, rats, hamsters and guinea pigs), mammals of commercial significance (such as horses, cows, camels, pigs and sheep), and also companion mammals (such as domestic animals, e.g., rabbits, ferrets, dogs, and cats). A RAB7L1, a LRRK2, or a VPS35 molecule can comprise a protein encoded by a nucleic acid sequence homologous to the human nucleic acid, wherein the nucleic acid is found in a different species and wherein that homolog encodes a protein similar to a RAB7L1, a LRRK2, or a VPS35 protein.

The invention utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “DNA Cloning: A Practical Approach.” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (2001).

One skilled in the art can obtain RAB7L1, a LRRK2, or a VPS35 molecule, in several ways, which include, but are not limited to, isolating the protein via biochemical means or expressing a nucleotide sequence encoding the protein of interest by genetic engineering methods.

The invention provides for a RAB7L1, a LRRK2, or a VPS35 molecule that are encoded by nucleotide sequences. The RAB7L1. LRRK2, or VPS35molecule can be a polypeptide encoded by a nucleic acid (including genomic DNA, complementary DNA (cDNA), synthetic DNA, as well as any form of corresponding RNA). For example, a RAB7L1, a LRRK2, or a VPS35 molecule can be encoded by a recombinant nucleic acid encoding a human RAB7L1, a human LRRK2, or a human VPS35 protein, or fragment thereof. The RAB7L1, LRRK2, or VPS35 molecules of the invention can be obtained from various sources and can be produced according to various techniques known in the art. For example, a nucleic acid that encodes a RAB7L1, a LRRK2, or a VPS35 molecule can be obtained by screening DNA libraries, or by amplification from a natural source. The RAB7L1, LRRK2, or VPS35 molecule of the invention can be produced via recombinant DNA technology and such recombinant nucleic acids can be prepared by conventional techniques, including chemical synthesis, genetic engineering, enzymatic techniques, or a combination thereof. A RAB7L1, a LRRK2, or a VPS35 molecule of this invention can also encompasses variants of the human RAB7L1, LRRK2, or VPS35 proteins. The variants can comprise naturally-occurring variants due to allelic variations between individuals (e.g., polymorphisms), mutated alleles, or alternative splicing forms.

In one embodiment, a fragment of a nucleic acid sequence that comprises a RAB7L1, a LRRK2, or a VPS35 molecule can encompass any portion of at least about 8 consecutive nucleotides of SEQ ID NO: 1, 2, 3, 4, 5, 9, 10, 12, or 13. In one embodiment, the fragment can comprise at least about 10 nucleotides, at least about 15 nucleotides, at least about 20 nucleotides, or at least about 30 nucleotides of SEQ ID NO: 1, 2, 3, 4, 5, 9, 10, 12, or 13. Fragments include all possible nucleotide lengths between about 8 and about 100 nucleotides, for example, lengths between about 15 and about 100 nucleotides, or between about 20 and about 100 nucleotides.

A RAB7L1, a LRRK2, or a VPS35 molecule, can be a fragment of a RAB7L1, a LRRK2, or a VPS35 protein. For example, the RAB7L1, LRRK2, or VPS35 protein fragment can encompass any portion of at least about 8 consecutive amino acids of SEQ ID NO: 6, 7, 8, 1, 14, 26, 27, or 28. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, at least about 80 consecutive amino acids, at least about 90 consecutive amino acids, at least about 100 consecutive amino acids, at least about 110 consecutive amino acids, or at least about 120 consecutive amino acids of SEQ ID NOS: 6, 7, 8, 11, 14, 26, 27, or 28. Fragments include all possible amino acid lengths between about 8 and 80 about amino acids, for example, lengths between about 10 and about 80 amino acids, between about 15 and about 80 amino acids, between about 20 and about 80 amino acids, between about 35 and about 80 amino acids, between about 40 and about 80 amino acids, between about 50 and about 80 amino acids, or between about 70 and about 80 amino acids.

Methods of Treating Parkinson's Disease

In one aspect, the invention provides a method of treating Parkinson's Disease (PD) in a subject comprising: (a) determining the presence or absence of a genetic variant at the PARK16 and LRRK2 loci in a sample from a subject, wherein the presence of a PD-associated genetic variant at both the PARK16 and LRRK2 loci in the subject sample indicates the subject has an increased risk or predisposition to PD, and (b) administering a treatment if the subject has an increased risk or predisposition to PD.

In another aspect, the invention provides a method of treating Parkinson's Disease (PD) in a subject comprising: (a) determining the presence or absence of a genetic variant at the LRRK2 locus in a sample from a subject, wherein the presence of a PD-associated genetic variant at the LRRK2 locus in the subject sample indicates the subject has an increased risk or predisposition to PD, and (b) administering a treatment if the subject has an increased risk or predisposition to PD.

In another aspect, the invention provides a method of treating Parkinson's Disease (PD) in a subject comprising: (a) determining the presence or absence of a genetic variant at the PARK16 locus in a sample from a subject, wherein the presence of a PD-associated genetic variant at the PARK16 locus in the subject sample indicates the subject has an increased risk or predisposition to PD, and (b) administering a treatment if the subject has an increased risk or predisposition to PD.

As used herein. “single-nucleotide polymorphism” or “SNP” refers to variations at single-nucleotide positions in the DNA sequence among individuals. Information on SNPs can be found in publically accessible databases, such as, in the SNP database at the National Center for Biotechnology Information (NCBI) (http://www.ncbi.nlm.nih.gov/). In one embodiment, the genetic variant at the PARK 16 locus comprises single-nucleotide polymorphism (SNP) rs823114, SNP rs823154, SNP rs823128, SNP rs947211, or a combination thereof.

In one embodiment, the PARK16 locus comprises the genes SLC45A3, NUCKS1, RAB7L1, SLC41A1, and PM20D1. In one embodiment, the genetic variant at the PARK 16 locus comprises a genetic variant in the RAB7L1 gene. In another embodiment, the genetic variant at the PARK 16 locus comprises a genetic variant at the SLC45A3, NUCKS1, SLC41A1, or PM20D1 gene. In one embodiment, the genetic variant at the RAB7L1 gene is SNP rs1572931.

Without being bound by theory, genetic variants can be associated with PD. In one embodiment, the PD-associated genetic variant at the PARK16 locus comprises a guanine (G) nucleotide at SNP rs1572931.

Genetic variants can also affect the splicing of mRNA. Without being bound by theory, pre-mRNA transcribed from genomic DNA can be spliced so that introns are removed and exons are joined together. Transcribed pre-mRNA can be alternatively spliced creating a range of unique proteins (known as “isoforms”) and/or mRNAs (known as “transcript variants”) by varying the exon composition of the mRNA. In one embodiment, the PD-associated genetic variant at the PARK16 locus encodes a RAB7L1 mRNA, wherein exon 2 is excluded from the RAB7L1 mRNA sequence. In one embodiment, the PD-associated genetic variant at the PARK16 locus results in loss of expression of a RAB7L1 protein. Various mutations that affect the transcription and translation of a RAB7L1 molecule can result in loss of expression of a RAB7L1 protein.

In one embodiment, the genetic variant at the LRRK2 locus comprises SNP rs1176052. In another embodiment, the PD-associated genetic variant at the LRRK2 locus encodes the protein of SEQ ID NO: 27 or 28. In another embodiment, the protein of SEQ ID NO: 27 or 28 is associated with familial PD. In another embodiment, the genetic variant at the LRRK2 locus is associated with sporadic, or non-familial PD.

In one embodiment, the PD-associated genetic variant at the LRKK2 locus results in loss of expression of a LRKK2 protein. Various mutations that affect the transcription and translation of a LRRK2 molecule can result in loss of expression of a LRRK2 protein.

In one aspect the invention provides, a method of treating PD in a subject comprising: (a) measuring the expression levels of a set of genes in a sample from a subject, wherein the set of genes comprises at least one gene selected from the genes listed in Table 2 (b) comparing the expression levels of the set of genes in the subject sample to expression levels of the same set of genes in a reference sample or samples, wherein the reference sample or samples are from an individual who has a PD-associated SNP, and wherein similar expression levels of the set of genes in the subject sample and the set of genes in the reference sample(s) indicates the subject has an increased risk or predisposition to PD, and (c) administering a treatment if the subject has an increased risk or predisposition to PD.

In another aspect, the invention provides a method of treating PD in a subject comprising: (a) determining a level of full-length RAB7L1 in a sample from a subject, (b) comparing the level of full-length RAB7L1 from the subject sample to a full-length RAB7L1 level in a reference sample, wherein the reference sample is associated with a non-PD status, and wherein a reduced level of the full-length RAB7L in the subject sample indicates the subject has an increased risk or predisposition to PD, and (c) administering a treatment if the subject has an increased risk or predisposition to PD. In one embodiment, the level of full-length RAB7L is protein level of full-length RAB7L, or mRNA levels of the full-length RAB7L, or a combination thereof.

In another aspect, the invention provides a method of treating Parkinson's Disease (PD) in a subject comprising: (a) determining a level of isoform 3 of RAB7L1 in a sample from a subject, (b) comparing the level of isoform 3 of RAB7L1 from the subject sample to an isoform 3 of RAB7L1 level in a reference sample, wherein the reference sample is associated in non-PD status, and wherein an increased level of isoform 3 of RAB7L1 in the subject sample indicates the subject has an increased risk or predisposition to PD, and (c) administering a treatment if the subject has an increased risk or predisposition to PD. In one embodiment, the level of isoform 3 of RAB7L1 is a protein level. In one embodiment, the method further comprises determining the level of transcript variant 4, 5, or a combination thereof of RAB7L1.

A method of treating Parkinson's Disease (PD) in a subject comprising: (a) determining a level of transcript variant 4, 5, or a combination thereof of RAB7L1 in a sample from a subject, (b) comparing the level of transcript variant 4, 5, or a combination thereof of RAB7L1 from the subject sample to a transcript variant 4, 5, or a combination thereof of RAB7L1 level in a reference sample, wherein the reference sample is associated in non-PD status, and wherein an increased level of transcript variant 4, 5, or a combination thereof of RAB7L1 in the subject sample indicates the subject has an increased risk or predisposition to PD, and (c) administering a treatment if the subject has an increased risk or predisposition to PD. In one embodiment, the level of transcript variant 4, 5, or a combination thereof of RAB7L1 is a mRNA level. In another embodiment, the method further comprises determining the level of isoform 3 of RAB7L1.

In one embodiment, the invention provides for determine the level of retromer components. Without being bound buy theory, retromer is a complex of proteins which are involved in recycling between the endolysosomal compartment of a cell and the Golgi apparatus. In mammals, proteins of the retromer complex include, but are not limited to Vps26, Vps29, Vps35, SNX1, SNX2, SNX5 and SNX6. The retromer complex can act in two subcomplexes; a cargo recognition complex that comprises Vps35, Vps29 and Vps26 (Vps trimer), and SNX-BAR dimers that comprises SNX1 and SNX2 or SNX5 and SNX6.

In another aspect, the invention provides a method of treating Parkinson's Disease (PD) in a subject comprising: (a) determining a level of retromer components in a sample from a subject, (b) comparing the level of retromer components from the subject sample to a retromer component level in a reference sample, wherein the reference sample is associated with a non-PD status, and wherein a reduced level of the retromer components in the subject sample indicates the subject has an increased risk or predisposition to PD, and (c) administering a treatment if the subject has an increased risk or predisposition to PD. In one embodiment, the level of retromer component is protein level of retromer component, or mRNA levels of retromer component, or a combination thereof. In another embodiment, the retromer component is VPS35, VPS29, VPS26 or a combination thereof. In a further embodiment the retromer component is SNX1, SNX2. SNX5, SNX6, or a combination thereof. In one embodiment, the level of VPS35. VPS29, or VPS26 is protein level of VPS35, VPS29, or VPS26, or mRNA levels of VPS35, VPS29, or VPS26, or a combination thereof.

In one aspect, the invention provides a method of treating PD in a subject. In one embodiment, the treatment comprises administering to the subject an effective amount of a nucleic acid encoding a protein comprising 90% of SEQ ID NO: 6, SEQ ID NO: 26, SEQ ID NO: 11, SEQ ID NO: 14, or a combination or fragment thereof. In another embodiment the treatment comprises administering to the subject an effective amount of a protein comprising 90% of SEQ ID NO: 6, SEQ ID NO: 26. SEQ ID NO: 11, SEQ ID NO: 14, or a combination or fragment thereof. In one embodiment, the protein comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of SEQ ID NO: 6, SEQ ID NO: 26, SEQ ID NO: 11, or SEQ ID NO: 14.

In one embodiment, the treatment comprises administering to the subject an effective amount of a nucleic acid encoding a protein comprising 90% of SEQ ID NO: 6, SEQ ID NO: 26. SEQ ID NO: 14, or a combination or fragment thereof. In another embodiment, the treatment comprises administering to the subject an effective amount of a protein comprising 90% of SEQ ID NO: 6, SEQ ID NO: 26, SEQ ID NO: 14, or a combination or fragment thereof. In one embodiment, the protein comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of SEQ ID NO: 6, SEQ ID NO: 26, or SEQ ID NO: 14.

In one embodiment, the treatment comprises administering to the subject an effective amount of a nucleic acid encoding a protein comprising 90% of SEQ ID NO: 11, SEQ ID NO: 14, or a combination or fragment thereof. In another embodiment, the treatment comprises administering to the subject an effective amount of a protein comprising 90% of SEQ ID NO: 11, SEQ ID NO: 14, or a combination or fragment thereof. In one embodiment, the protein comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of SEQ ID NO: 6, SEQ ID NO: 26, SEQ ID NO: 11, or SEQ ID NO: 14.

In one embodiment, the PD disease status is determined by any suitable method, including but not limited to a physical examination of the subject, a neurological examination of the subject, a brain scan, or a combination thereof. Suitable methods for determining the PD disease statuts are known to one of skill in the art.

In one embodiment, the subject is not diagnosed with PD. In another embodiment, the subject is diagnosed with PD. In another embodiment, the subject is diagnosed with a pre-disease prodromal state.

In one embodiment, the method further comprises a physical examination of the subject, a neurological examination of the subject, a brain scan, or a combination thereof. Methods and types of physical examinations are known to one of skill in the art.

In one embodiment, the method further comprises a step of sequencing nucleic acids isolated from the subject's sample to determine the presence or absence of a PD-risk associated SNP, wherein the presence of a PD-risk associated SNP is further indicative that the subject is at risk of developing PD or is suffering from PD.

In one embodiment, the subject is suspected of having PD or is at risk of developing PD based on the presence of any one of parkinsonism symptoms. Determination of parkinsonism symptoms are known to one of skill in the art.

In one embodiment, the sample is a CSF sample, blood sample, plasma sample, serum sample or any combination thereof. Methods of sample collection are known to one of skill in the art.

Expression Systems and Purification of Recombinant Proteins One skilled in the art understands that polypeptides (for example RAB7L1, LRRK2, or VPS35, and the like) can be obtained in several ways, which include but are not limited to, expressing a nucleotide sequence encoding the protein of interest, or fragment thereof, by genetic engineering methods.

In one embodiment, the nucleic acid is expressed in an expression cassette, for example, to achieve overexpression in a cell. The nucleic acids of the invention can be an RNA, cDNA, cDNA-like, or a DNA of interest in an expressible format, such as an expression cassette, which can be expressed from the natural promoter or an entirely heterologous promoter. The nucleic acid of interest can encode a protein, and may or may not include introns. Any recombinant expression system can be used, including, but not limited to, bacterial, mammalian, yeast, insect, or plant cell expression systems.

Host cells transformed with a nucleic acid sequence encoding a RAB7L1, a LRRK2, or a VPS35 molecule can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a RAB7L1, a LRRK2, or a VPS35 molecule can be designed to contain signal sequences which direct secretion of soluble polypeptide molecules encoded by a RAB7L1, a LRRK2, or a VPS35 molecule, through a prokaryotic or eukaryotic cell membrane.

Nucleic acid sequences comprising a RAB7L1, a LRRK2, or a VPS35 molecule that encode a polypeptide can be synthesized, in whole or in part, using chemical methods known in the art. Alternatively, a RAB7L1, a LRRK2, or a VPS35 molecule can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431 A Peptide Synthesizer (Perkin Elmer). Optionally, fragments of a RAB7L1, a LRRK2, or a VPS35 molecule can be separately synthesized and combined using chemical methods to produce a full-length molecule.

A synthetic peptide can be substantially purified via high performance liquid chromatography (HPLC). The composition of a synthetic RAB7L1, LRRK2, or VPS35 molecule can be confirmed by amino acid analysis or sequencing. Additionally, any portion of an amino acid sequence comprising a protein encoded by a RAB7L1, a LRRK2, or a VPS35 molecule can be altered during direct synthesis and/or combined using chemical methods with sequences from other proteins to produce a variant polypeptide or a fusion protein.

The invention further encompasses methods for using a protein or polypeptide encoded by a nucleic acid sequence of a RAB7L1, a LRRK2, or a VPS35 molecule, such as the sequences shown in SEQ ID NOS: 6, 7, 8, 11, 14, 26, 27, or 28. In another embodiment, the polypeptide can be modified, such as by glycosylations and/or acetylations and/or chemical reaction or coupling, and can contain one or several non-natural or synthetic amino acids. An example of a RAB7L1, a LRRK2, or a VPS35 molecule has the amino acid sequence shown in either SEQ ID NO: 6, 7, 8, 11, 14, 26, 27, or 28. In certain embodiments, the invention encompasses variants of a human protein encoded by a RAB7L1, a LRRK2, or a VPS35 molecule.

One skilled in the art understands that expression of desired protein products can be carried out in prokaryotes (e.g. E. coli and B. subtilis), in plant cell systems infected with recombinant virus expression vectors (e.g., tobacco mosaic virus, TMV: cauliflower mosaic virus, CaMV), in insect cells (e.g. Autographa californica nuclear polyhedrosis virus (AcNPV) can be used as a vector to express foreign genes in Spodoptera frugiperda cells or in Trichoplusia larvae), in yeast cells (e.g. Saccharomyces sp., Pichia sp.), or in mammalian cells (e.g. BHK cells, VERO cells, CHO cells and the like).

Expression vectors (also known in the art as fusion-vectors) can be designed to add a number of amino acid residues, usually to the N-terminus of the expressed recombinant protein. Such fusion vectors can serve three functions: 1) to increase the solubility of the desired recombinant protein; 2) to increase expression of the recombinant protein of interest; and 3) to aid in recombinant protein purification by acting as a ligand in affinity purification.

An exogenous nucleic acid can be introduced into a cell via a variety of techniques known in the art, such as lipofection, microinjection, calcium phosphate or calcium chloride precipitation, DEAE-dextrin-mediated transfection, or electroporation. Electroporation is carried out at approximate voltage and capacitance to result in entry of the DNA construct(s) into cells of interest. Other methods used to transfect cells can also include modified calcium phosphate precipitation, polybrene precipitation, liposome fusion, and receptor-mediated gene delivery.

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized.

A RAB7L1, a LRRK2, or a VPS35 molecule can be purified from any human or non-human cell which expresses the polypeptide, including those which have been transfected with expression constructs that express a RAB7L1, a LRRK2, or a VPS35 molecule. A purified RAB7L1, LRRK2, or VPS35 molecule can be separated from other compounds which normally associate with the RAB7L1, LRRK2, or VPS35 molecules, in the cell, such as certain proteins, carbohydrates, or lipids, using methods practiced in the art. The desired polypeptide molecule (for example, a RAB7L1, a LRRK2, or a VPS35 molecule) is isolated or purified away from contaminating soluble proteins and polypeptides by suitable purification techniques. Non-limiting purification methods for proteins include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

Methods of Administration

Nucleic Acid Delivery Methods.

The invention provides methods for treating Parkinson's Disease (PD) in a subject. In one embodiment, the method can comprise administering to the subject a RAB7L1, a LRRK2, or a VPS35 molecule (e.g, a RAB7L1, a LRRK2, or a VPS35 polypeptide or a RAB7L1, a LRRK2, or a VPS35 polynucleotide).

Various approaches can be carried out to restore the activity or function of a RAB7L1, a LRRK2, or a VPS35 molecule in a subject, such as those carrying an genetic variant in a RAB7L1, a LRRK2, or a VPS35 gene locus. For example, supplying wild-type RAB7L1. LRRK2, or VPS35 gene function to such subjects can treat Parkinson's Disease. Increasing a RAB7L1, a LRRK2, or a VPS35 gene expression level or activity can be accomplished through gene or protein therapy.

A nucleic acid encoding a RAB7L11, a LRRK2, or a VPS35 molecule can be introduced into the cells of a subject. For example, the wild-type gene (or fragment thereof) can also be introduced into the cells of the subject in need thereof using a vector as described herein. The vector can be a viral vector or a plasmid. The gene can also be introduced as naked DNA. The gene can be provided so as to integrate into the genome of the recipient host cells, or to remain extra-chromosomal. Integration can occur randomly or at precisely defined sites, such as through homologous recombination. For example, a functional copy of a RAB7L1, a LRRK2, or a VPS35 molecule can be inserted in replacement of an altered version in a cell, through homologous recombination. Further techniques include gene gun, liposome-mediated transfection, or cationic lipid-mediated transfection. Gene therapy can be accomplished by direct gene injection, or by administering ex vivo prepared genetically modified cells expressing a functional polypeptide.

Delivery of nucleic acids into viable cells can be effected ex vivo, in situ, or in vivo by use of vectors, and more particularly viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (see, for example. Anderson, Nature, supplement to vol. 392, no. 6679, pp. 25-20 (1998)). Introduction of a nucleic acid or a gene encoding a polypeptide of the invention can also be accomplished with extrachromosomal substrates (transient expression) or artificial chromosomes (stable expression). Cells may also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., (1992)J Gen Virol. 73(Pt 6):1533-6), adenovirus (Berkner (1992) Curr Top Microbiol Immunol. 158:39-66; Berkner (1988) Biotechniques, 6(7):616-29; Gorziglia and Kapikian (1992). J Virol. 66(7):4407-12; Quantin et al., (1992) Proc Natl Acad Sci USA. 89(7):2581-4; Rosenfeld et al., (1992) Cell. 68(1):143-55; Wilkinson et al., (1992) Nucleic Acids Res. 20(9):2233-9; Stratford-Perricaudet et al., (1990) Hum Gene Ther. 1(3):241-56), vaccinia virus (Moss (1992) Curr Opin Biotechnol. 3(5):518-22), adeno-associated virus (Muzyczka, (1992) Curr Top Microbiol Immunol. 158:97-129; Ohi et al., (1990) Gene. 89(2):279-82), herpesviruses including HSV and EBV (Margolskee (1992) Curr Top Microbiol Immunol. 158:67-95; Johnson et al., (1992) Brain Res Mol Brain Res.12(1-3):95-102; Fink et al., (1992) Hum Gene Ther. 3(1):11-9; Breakefield and Geller (1987) Mol Neurobiol. 1(4):339-71; Freese et al., (1990) Biochem Pharmacol. 40(10):2189-99), and retroviruses of avian (Bandyopadhyay and Temin (1984) Mol Cell Biol. 4(4):749-54; Petropoulos et al., (1992) J Virol. 66(6):3391-7), murine (Miller et al. (1992) Mol Cell Biol. 12(7):3262-72; Miller et al., (1985) J Virol. 55(3):521-6; Sorge et al., (1984) Mol Cell Biol. 4(9):1730-7; Mann and Baltimore (1985)J Virol. 54(2):401-7; Miller et al., (1988)J Virol. 62(11):4337-45), and human origin (Shimada et al., (1991) J Clin Invest. 88(3):1043-7; Helseth et al., (1990) J Virol. 64(12):6314-8; Page et al., (1990)J Virol. 64(11):5270-6; Buchschacher and Panganiban (1992) J Virol. 66(5):2731-9).

Non-limiting examples of in vivo gene transfer techniques include transfection with viral (typically retroviral) vectors (see U.S. Pat. No. 5,252,479, which is incorporated by reference in its entirety) and viral coat protein-liposome mediated transfection (Dzau et al., Trends in Biotechnology 11:205-210 (1993), incorporated entirely by reference). For example, naked DNA vaccines are generally known in the art; see Brower, Nature Biotechnology, 16:1304-1305 (1998), which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al., Science 256:808-813 (1992); U.S. Pat. Nos. 5,252,479, 5,747,469, 6,017,524, 6,143,290, 6,410,010 6,511,847; 8,398,968; and 8,404,653 which are all hereby incorporated by reference in their entireties. For an example of gene therapy treatment in humans see Porter et al., NEJM 2011 365:725-733 and Kalos et al. Sci. Transl. Med. 2011. 201 3(95):95. For additional reviews of gene therapy technology, see Friedmann, Science, 244:1275-1281 (1989); Verma, Scientific American: 68-84 (1990); Miller, Nature, 357: 455-460 (1992); Kikuchi et al., J Dermatol Sci. 2008 May; 50(2):87-98; Isaka et al., Expert Opin Drug Deliv. 2007 September; 4(5):561-71; Jager et al., Curr Gene Ther. 2007 August; 7(4):272-83; Waehler et al., Nat Rev Genet. 2007 August; 8(8):573-87; Jensen et al., Ann Med. 2007; 39(2):108-15; Herweijer et al., Gene Ther. 2007 January; 14(2):99-107; Eliyahu et al., Molecules, 2005 Jan. 31; 10(1):34-64; and Altaras et al., Adv Biochem Eng Biotechnol. 2005; 99:193-260, all of which are hereby incorporated by reference in their entireties.

These methods described herein are by no means all-inclusive, and further methods to suit the specific application is understood by the ordinary skilled artisan. Moreover, the effective amount of the compositions can be further approximated through analogy to compounds known to exert the desired effect.

Protein Delivery Methods.

Protein replacement therapy can increase the amount of protein by exogenously introducing wild-type or biologically functional protein by way of infusion. A replacement polypeptide can be synthesized according to known chemical techniques or may be produced and purified via known molecular biological techniques. Protein replacement therapy has been developed for various disorders. For example, a wild-type protein can be purified from a recombinant cellular expression system (e.g., mammalian cells or insect cells-see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

A RAB7L1, a LRRK2, or a VPS35 molecule can also be delivered in a controlled release system. For example, the RAB7L1, LRRK2, or VPS35 molecule can be administered using intravenous infusion, an implantable osmotic pump, a transdermal patch, liposomes, or other modes of administration. In one embodiment, a pump can be used (see Sefton (1987) Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science (1990) 249:1527-1533).

Pharmaceutical Compositions and Methods of Administration

In some embodiments, a RAB7L1, a LRRK2, or a VPS35 molecule can be supplied in the form of a pharmaceutical composition, comprising an isotonic excipient prepared under sufficiently sterile conditions for human administration. Choice of the excipient and any accompanying elements of the composition comprising a RAB7L1, a LRRK2, or a VPS35 molecule will be adapted in accordance with the route and device used for administration. In some embodiments, a composition comprising a RAB7L1, a LRRK2, or a VPS35 molecule can also comprise, or be accompanied with, one or more other ingredients that facilitate the delivery or functional mobilization of the RAB7L1, LRRK2, or VPS35 molecule.

These methods described herein are by no means all-inclusive, and further methods to suit the specific application is understood by the ordinary skilled artisan. Moreover, the effective amount of the compositions can be further approximated through analogy to compounds known to exert the desired effect.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A RAB7L1, a LRRK2, or a VPS35 molecule can be administered to the subject one time (e.g., as a single injection or deposition). Alternatively, a RAB7L1, a LRRK2, or a VPS35 molecule can be administered once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. Furthermore, a RAB7L1, a LRRK2, or a VPS35 molecule can be co-administrated with another therapeutic.

In one embodiment, a RAB7L1, a LRRK2, or a VPS35 molecule can be co-administrated with a Parkinson's Disease drug. Some non-limiting examples of conventional PD drugs include: levodopa, carbidopa/levodopa (co-careldopa), benserazide/levodopadopamine (co-beneldopa), dopamine agonists (e.g., bromocriptine, pergolide, pramipexole, ropinirole, piribedil, cabergoline, apomorphine, and lisuride), MAO-B inhibitors (e.g. selegiline, and rasagiline), amantadine, and anticholingerics.

A RAB7L1, a LRRK2, or a VPS35 molecule may also be used in combination with surgical or other interventional treatment regimens used for the treatment of PD.

A RAB7L1, a LRRK2, or a VPS35 molecule can be administered to a subject by any means suitable for delivering the protein, nucleic acid or compound to cells of the subject. For example, it can be administered by methods suitable to transfect cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with PD by any means that produce contact of the active ingredient with the agent's site of action in the body of a human or non-human subject. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa. (20^{th} ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers, such as PBS, Hank's solution, or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™(BASF, Parsippany. N.J.) or phosphate buffered saline (PBS). The composition must be sterile and fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the RAB7L1, LRRK2, or VPS35 molecule in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art

A composition of the invention can be administered to a subject in need thereof. Subjects in need thereof can include but are not limited to, for example, a mammal such as a dog, a cat, a cow, a horse, a rabbit, a monkey, a pig, a sheep, a goat, or a human. A composition of the invention can also be formulated as a sustained and/or timed release formulation. Such sustained and/or timed release formulations can be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5.073.543; 5,639,476; 5,354,556; and 5,733,566, the disclosures of which are each incorporated herein by reference. The pharmaceutical compositions of the invention (e.g., that have a therapeutic effect) can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, can be readily selected for use with the pharmaceutical compositions of the invention. Single unit dosage forms suitable for oral administration, such as, but not limited to, tablets, capsules, gel-caps, caplets, or powders, that are adapted for sustained release are encompassed by the invention.

In the methods described herein, a RAB7L1, a LRRK2, or a VPS35 molecule, can be administered to the subject either as RNA, in conjunction with a delivery reagent, or as a nucleic acid (e.g., a recombinant plasmid or viral vector) comprising sequences which express the gene product. Suitable delivery reagents for administration of the a RAB7L1, a LRRK2, or a VPS35 molecule, include the Mirus Transit TKO lipophilic reagent; lipofectin; lipofectamine; cellfectin; or polycations (e.g., polylysine), or liposomes.

The dosage administered can be a therapeutically effective amount of the composition sufficient to result in treatment of PD, and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

In some embodiments, the effective amount of the administered RAB7L1, LRRK2, or VPS35 molecule is at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10 μg/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, at least about 1000 μg/kg body weight, at least about 1500 μg/kg body weight, at least about 2000 μg/kg body weight, at least about 2500 μg/kg body weight, at least about 3000 μg/kg body weight, at least about 3500 μg/kg body weight, at least about 4000 μg/kg body weight, at least about 4500 μg/kg body weight, at least about 5000 μg/kg body weight, at least about 5500 μg/kg body weight, at least about 6000 μg/kg body weight, at least about 6500 μg/kg body weight, at least about 7000 μg/kg body weight, at least about 7500 μg/kg body weight, at least about 8000 μg/kg body weight, at least about 8500 μg/kg body weight, at least about 9000 μg/kg body weight, at least about 9500 μg/kg body weight, or at least about 10000 μg/kg body weight.

In one embodiment, a RAB7L1, a LRRK2, or a VPS35 molecule is administered at least once daily. In another embodiment, a RAB7L1, a LRRK2, or a VPS35 molecule is administered at least twice daily. In some embodiments, a RAB7L1, a LRRK2, or a VPS35 molecule is administered for at least 1 week, for at least 2 weeks, for at least 3 weeks, for at least 4 weeks, for at least 5 weeks, for at least 6 weeks, for at least 8 weeks, for at least 10 weeks, for at least 12 weeks, for at least 18 weeks, for at least 24 weeks, for at least 36 weeks, for at least 48 weeks, or for at least 60 weeks. In further embodiments, a RAB7L1, a LRRK2, or a VPS35 molecule is administered in combination with a second therapeutic agent.

Toxicity and therapeutic efficacy of therapeutic compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic agents that exhibit large therapeutic indices are useful. Therapeutic compositions that exhibit some toxic side effects can be used.

Administration of a RAB7L1, a LRRK2, or a VPS35 molecule is not restricted to a single route, but may encompass administration by multiple routes. Multiple administrations may be sequential or concurrent. Other modes of application by multiple routes will be apparent to one of skill in the art.

Methods of Detection

Embodiments of the invention provide for detecting expression of a RAB7L1, a LRRK2, or a VPS35 molecule. In one embodiment, a gene alteration can result in increased or reduced protein expression and/or activity. The alteration can be determined at the level of the DNA, RNA, or polypeptide.

In some embodiments, the detecting comprises detecting in a biological sample whether there is a reduction in an mRNA encoding a RAB7L1, a LRRK2, or a VPS35 protein, or a reduction in a RAB7L1, a LRRK2, or a VPS35 protein, or a combination thereof. In further embodiments, the detecting comprises detecting in a biological sample whether there is a reduction in an mRNA encoding a RAB7L1, a LRRK2, or a VPS35 protein, or a reduction in a RAB7L1, a LRRK2, or a VPS35 protein, or a combination thereof. The presence of such an alteration is indicative of the presence or predisposition to PD.

Methods for detecting and quantifying RAB7L1, LRRK2, or VPS35 molecules in biological samples are known the art. For example, protocols for detecting and measuring the expression of a polypeptide encoded by a RAB7L1, a LRRK2, or a VPS35 molecule, using either polyclonal or monoclonal antibodies specific for the polypeptide are well established. Non-limiting examples include Western blot, enzyme-linked immunosorbent assay (ELISA), radioimmunoassay (RIA), and fluorescence activated cell sorting (FACS).

In one embodiment, a biological sample comprises, a blood sample, serum, cells (including whole cells, cell fractions, cell extracts, and cultured cells or cell lines), tissues (including tissues obtained by biopsy), body fluids (e.g., urine, sputum, amniotic fluid, synovial fluid), or from media (from cultured cells or cell lines). In one embodiment, the sample is a CSF sample, a blood sample, a plasma sample, a serum sample, or any combination thereof. The methods of detecting or quantifying RAB7L1, LRRK2, or VPS35 molecules include, but are not limited to, amplification-based assays with (signal amplification) hybridization based assays and combination amplification-hybridization assays.

Any suitable biological sample can be used in the instant methods. The biological sample can be taken from body fluid, such as urine, saliva, bone marrow, blood, and derivative blood products (sera, plasma, PBMC, circulating cells, circulating RNA). The biological sample can be taken from a human subject, from an animal, or from a cell culture. The biological sample can be obtained in vivo, in vitro or ex vivo. Non-limiting examples of biological samples include blood, serum, plasma, cerebrospinal fluid, mucus, tissue, cells, and the like, or any combination thereof. In a non-limiting embodiment the biological sample is blood. In a non-limiting embodiment the biological sample is serum. In a non-limiting embodiment the biological sample is plasma. Any suitable method to isolate nucleic acids from biological samples are contemplated for use in the invention. Biological samples for analysis are stored under suitable conditions. In non-limiting examples biological samples are kept at about 4° C. In non-limiting examples biological samples are kept at about −20° C. In non-limiting examples biological samples are kept at about −70-80° C.

A RAB7L1, a LRRK2, or a VPS35 molecule can be determined at the nucleic acid level. Optionally, detection can be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (for example, see Jones et al, (2000) Hum Genet., 106(6):663-8), or a combination thereof. In one embodiment, the detection or determination comprises nucleic acid sequencing, selective hybridization, selective amplification, gene expression analysis, or a combination thereof. In one embodiment, the detection is performed by sequencing all or part of a RAB7L1, a LRRK2, or a VPS35 molecule, or by selective hybridization or amplification of all or part of the RAB7L1 LRRK2, or VPS35 molecule. A nucleic acid specific amplification can be carried out before the quantification step. In one embodiment, the detecting comprises using a northern blot; real time PCR and primers directed to SEQ ID NO: 1, 2, 3, 4, 5, 9, 10, 12, or 13; a ribonuclease protection assay; a hybridization, amplification, or sequencing technique to detect a RAB7L1, a LRRK2, or a VPS35 molecule; or a combination thereof. In another embodiment, the PCR primers comprise at least 10, at least 11, at least 12, at least 13, at least 14, at least 15, at least 16, at least 17, at least 18, at least 19, at least 20 consecutive nucleotides comprising SEQ ID NO: 1, 2, 3, 4, 5, 9, 10, 12, 13, 15, 16, 17, 18, 19, 24, 25, or a combination of the primers.

Hybridization detection methods are based on the formation of specific hybrids between complementary nucleic acid. A detection technique involves the use of a nucleic acid probe specific for the presence of a RAB7L1, a LRRK2, or a VPS35 molecule, followed by the detection of the presence of a hybrid. The probe can be in suspension or immobilized on a substrate or support (for example, as in nucleic acid array or chips technologies). The probe can be labeled to facilitate detection of hybrids. In one embodiment, the probe according to the invention can comprise a nucleic acid directed to SEQ ID NO: 1, 2, 3, 4, 5, 9, 10, 12, or 13. In another embodiment, the probe that detects the presence of a RAB7L1, a LRRK2, or a VPS35 molecule comprises SEQ ID NO: 15, 16, 17, 18, 19, 24, or 25.

A guide to the hybridization of nucleic acids is found in e.g., Sambrook, ed., Molecular Cloning: A Laboratory Manual (3^{rd} Ed.), Vols. 1-3, Cold Spring Harbor Laboratory, 1989; Current Protocols In Molecular Biology, Ausubel, ed. John Wiley & Sons, Inc., New York, 2001; Laboratory Techniques In Biochemistry And Molecular Biology: Hybridization With Nucleic Acid Probes, Part I. Theory and Nucleic Acid Preparation, Tijssen, ed. Elsevier, N. Y., 1993.

Sequencing can be carried out using techniques well known in the art, using automatic sequencers. The sequencing can be performed on a RAB7L1, a LRRK2, or a VPS35 molecule. In another embodiment, the sequencing can be performed using SEQ ID NO: 24, or 25.

Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR based single-strand conformational polymorphism (SSCP). Amplification usually requires the use of specific nucleic acid primers, to initiate the reaction. In one embodiment, amplification comprises using forward and reverse PCR primers directed to SEQ ID NO: 1, 2, 3, 4, 5, 9, 10, 12, or 13. In certain subjects, the downregulation of a RAB7L1, a LRRK2, or a VPS35 molecule corresponds to a subject with PD. In one embodiment, amplification can comprise using forward and reverse PCR primers comprising at least 10, at least 11, at least 12, at least 13, at least 14, at least 15, at least 16, at least 17, at least 18, at least 19, at least 20 consecutive nucleotides comprising SEQ ID NO: 15, 16, 17, 18, 19, 24, or 25.

Non-limiting amplification methods include, e.g., polymerase chain reaction. PCR (PCR Protocols. A Guide To Methods And Applications, ed. Innis, Academic Press, N. Y., 1990 and PCR Strategies, 1995, ed. Innis, Academic Press. Inc., N.Y.); ligase chain reaction (LCR) (Wu (1989) Genomics 4:560; Landegren (1988) Science 241:1077; Barringer (1990) Gene 89:117); transcription amplification (Kwoh (1989) PNAS 86:1173); and, self-sustained sequence replication (Guatelli (1990) PNAS 87:1874); Q Beta replicase amplification (Smith (1997) J. Clin. Microbiol. 35:1477-1491), automated Q-beta replicase amplification assay (Burg (1996) Mol. Cell. Probes 10:257-271) and other RNA polymerase mediated techniques (e.g., NASBA, Cangene, Mississauga, Ontario; see also Berger (1987) Methods Enzymol. 152:307-316; U.S. Pat. Nos. 4,683,195 and 4,683,202; and Sooknanan (1995) Biotechnology 13:563-564). All the references stated above are incorporated by reference in their entireties.

The invention provides for a nucleic acid primer, wherein the primer can be complementary to and hybridize specifically to a portion of a RAB7L1, a LRRK2, or a VPS35 molecule. Primers can be specific for a RAB7L1, a LRRK2, or a VPS35 molecule. By using such primers, the detection of an amplification product indicates the presence of a a RAB7L1, a LRRK2, or a VPS35 molecule. Examples of primers of this invention can be single-stranded nucleic acid molecules of about 8 to about 15 nucleotides in length. Perfect complementarity is useful to ensure high specificity; however, certain mismatch can be tolerated. For example, a nucleic acid primer or a pair of nucleic acid primers as described above can be used in a method for detecting the presence of a genetic variant in a subject. In one embodiment, primers can be used to detect the absence of reduced level of a RAB7L1, a LRRK2, or a VPS35 molecule. In some embodiments, the primers are directed to SEQ ID NO: 1, 2, 3, 4, 5, 9, 10, 12, or 13. In another embodiment, the PCR primers comprise at least 10, at least 11, at least 12, at least 13, at least 14, at least 15, at least 16, at least 17, at least 18, at least 19, at least 20 consecutive nucleotides comprising SEQ ID NO: 15, 16, 17, 18, 19, 24, or 25.

Compositions and Kits of the Invention

In one aspect, the invention provides a composition for evaluating the existence of, or predisposition to, PD in a subject, said composition comprising polynucleotides or oligonucleotides, wherein each polynucleotide or oligonucleotide hybridizes to a gene, gene fragment, or gene transcript of at least two different markers in a subject sample, wherein the markers comprise LRRK2, RAB7L1 and VPS35.

In another aspect, the invention provides a composition for evaluating the existence of, or predisposition to, PD in a subject, said composition comprising polynucleotides or oligonucleotides, wherein each polynucleotide or oligonucleotide hybridizes to a gene, gene fragment, or gene transcript of a different marker in a subject sample, each marker being one of the genes listed in Table 2.

In one embodiment, the composition comprises a microarray, a microfluidics card, a chip, or a chamber. In another aspect, the invention provides a diagnostic kit comprising the microarray, microfluidics card, chip, or chamber.

In another aspect, the invention provides a diagnostic kit for determining the levels of RAB7L1, LRRK2, VPS35, or a combination thereof, the kit comprising at least one oligonucleotide or polynucleotide to selectively quantify the levels of RAB7L1, LRRK2, VPS35, or a combination thereof. In one embodiment, the oligonucleotide or polynucleotide comprises SEQ ID NO: 15, 16, 17, or 18. In another embodiment, the oligonucleotide or polynucleotide comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of SEQ ID NO: 15, 16, 17, or 18.

In another aspect, the invention provides for a diagnostic kit for determining whether a sample from a subject exhibits a presence or absence of a PD-associated genetic variant, the kit comprising at least one oligonucleotide or polynucleotide for sequencing nucleic acids isolated from the subject's sample to determine the presence or absence of a PD-risk associated SNP, wherein the presence of a PD-risk associated SNP is further indicative that the subject is at risk of developing PD or is suffering from PD. In one embodiment, the oligonucleotide or polynucleotide comprises SEQ ID NO: 24, or 25. In another embodiment, the oligonucleotide or polynucleotide comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of SEQ ID NO: 24, or 25.

The kits of the invention may also include reagents necessary or useful for the amplification of target nucleic acids, which may include, but is not limited to, DNA polymerase enzymes, primer extension deoxynucleotide triphosphates, and any buffer or other solutions generally used in PCR amplification reactions and kits.

In one embodiment, the kit can further comprise reagents and/or protocols for performing a hybridization, or amplification. In one embodiment, the kit can comprise nucleic acid primers that specifically hybridize to and can prime a polymerase reaction from a RAB7L1, a LRRK2, or a VPS35 molecule comprising at least 10, at least 11, at least 12, at least 13, at least 14, at least 15, at least 16, at least 17, at least 18, at least 19, at least 20 consecutive nucleotides comprising SEQ ID NOS: 15, 16, 17, 18, 19, 24, or 25, or a combination of the primers. In one embodiment, primers can be used to detect the absence or reduction of a RAB7L1, a LRRK2, or a VPS35 molecule, such as a primer directed to SEQ ID NOS: 1, 2, 3, 4, 5, 9, 10, 12, or 13. In another embodiment, the PCR primer comprises at least 10, at least 11, at least 12, at least 13, at least 14, at least 15, at least 16, at least 17, at least 18, at least 19, at least 20 consecutive nucleotides comprising SEQ ID NOS: 15, 16, 17, 18, 19, 24, or 25. In some embodiments, the kit comprises a probe for detecting a RAB7L1, a LRRK2, or a VPS35 molecule.

The diagnosis methods can be performed in vitro, ex vivo, or in vivo. These methods utilize a sample from the subject in order to assess the status of a RAB7L1, a LRRK2, or a VPS35 molecule. The sample can be any biological sample derived from a subject, which contains nucleic acids or polypeptides. Examples of such samples include, but are not limited to, fluids, tissues, cell samples, organs, and tissue biopsies. Non-limiting examples of samples include blood, liver, plasma, serum, saliva, urine, or seminal fluid. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), centrifugation. The nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents, such as probes or primers, in order to assess the absence or presence of a RAB7L1, a LRRK2, or a VPS35 molecule. Contacting can be performed in any suitable device, such as a plate, tube, well, or glass. In some embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

These methods described herein are by no means all-inclusive, and further methods to suit the specific application will be apparent to the ordinary skilled artisan. Moreover, the effective amount of the compositions can be further approximated through analogy to compounds known to exert the desired effect.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### Examples

A number of Examples are provided below to facilitate a more complete understanding of the present invention. The following examples illustrate the exemplary modes of making and practicing the present invention. However, the scope of the invention is not limited to specific embodiments disclosed in these Examples, which are for purposes of illustration only, since alternative methods can be utilized to obtain similar results.

### Example 1

### LRRK2 and PARK16 PD Risk Variants Impart a Common Brain Transcriptome Impact

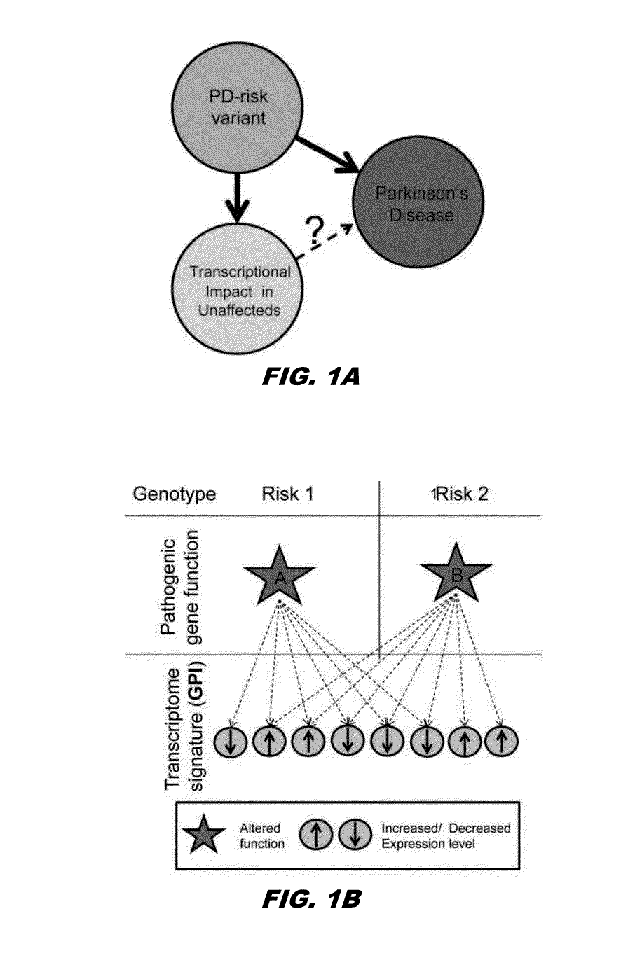

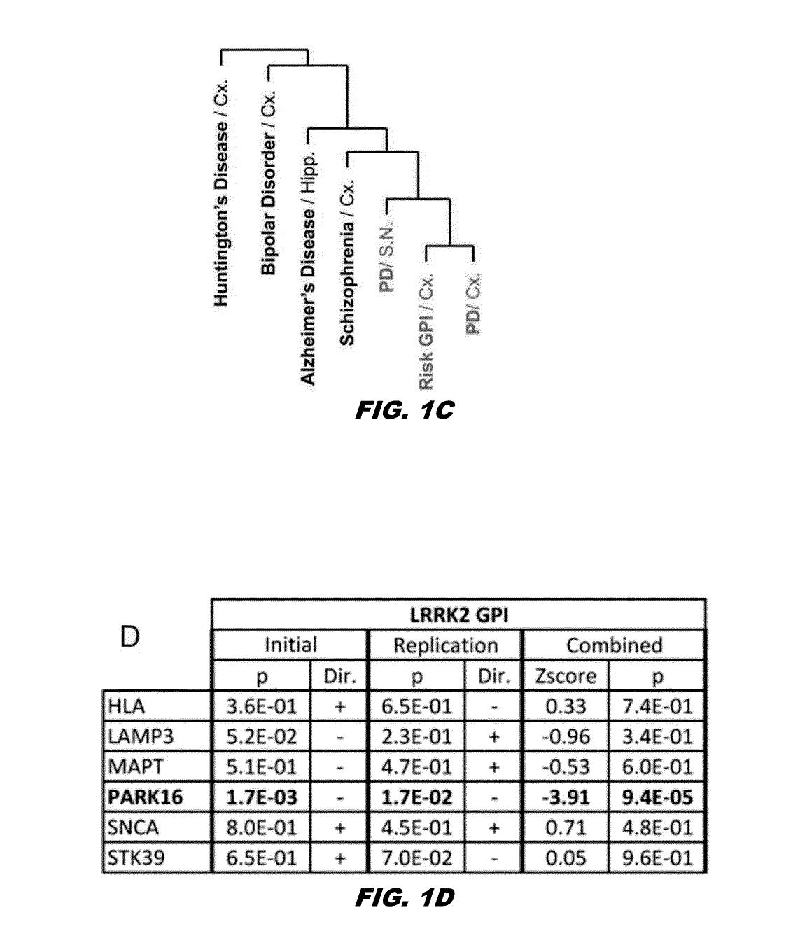

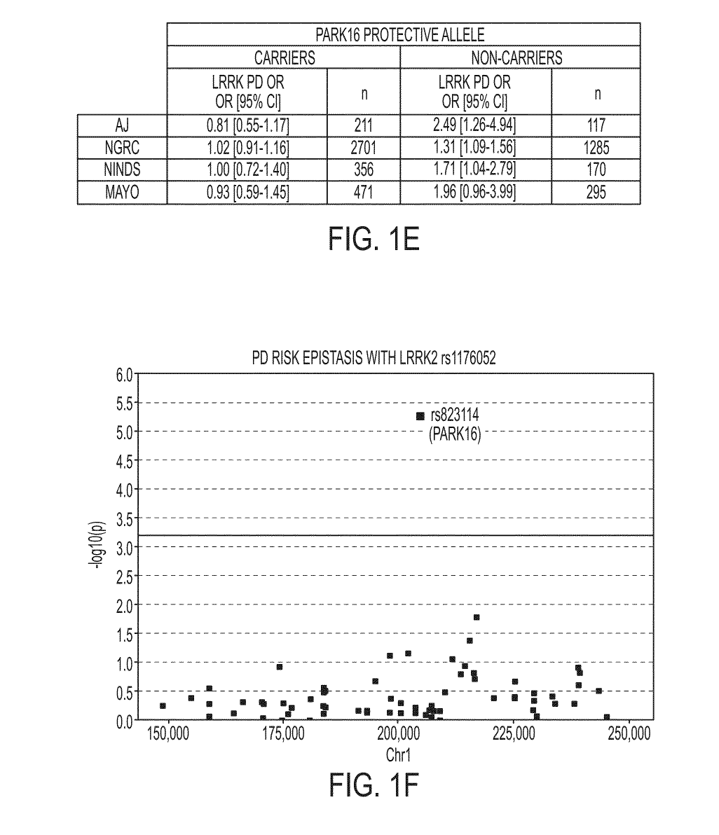

An unbiased and systematic approach was sought to assess the phenotypic impacts of common genetic variants associated with PD risk, particularly in brain tissue from yet unaffected carriers (FIG. 1A), in order to circumvent the limitations of the analysis of diseased patient autopsy tissue. To this end, the transcriptome-wide gene expression profiles of brain tissue samples from cohorts of unaffected individuals who share either a risk or a protective allele at any given PD risk SNP were compared (FIG. 1B). Such a Global Phenotypic Impact (GPI) quantifies the effect of disease risk variants onto the transcriptome-wide gene expression profile in brain. A key aspect of the GPI analysis herein is that tissue from unaffected individuals was tested, in hope of avoiding secondary effects of disease pathology such as cell loss.

The transcriptome-wide GPI at 7 PD-associated loci was assessed (SNCA, LRRK2, MAPT, HLA-DRA, PARK16, LAMP3, STK39, Table 1) (Simon-Sanchez et al., 2009) in a publically available gene expression dataset from cerebral cortex autopsy brain tissue of 185 individuals not apparently affected by a neurodegenerative disease (GSE15222).

TABLE 1
SNPs used for the GPI analysis and linkage with PD associated
SNPs identified by GWAS.

| | | PD- | Dis- | | | |
|---|---|---|---|---|---|---|
| | | associated | tance | | | |
| Locus | GPI SNPs | SNPs | (bp) | D′ | R | Source |
| LRRK2 | rs7306944 | rs2708453 | 42545 | 0.936 | 1 | PDGene |
| | | rs7304279 | 29835 | 0.878 | 0.937 | PDGene |
| MAPT | rs17563787 | rs1981997 | 243580 | 1 | 1 | PDGene |
| | | rs393152 | 94097 | 1 | 1 | PDGene |
| PARK16 | rs823128 | rs1620334 | 15533 | 1 | 1 | PDGene |
| | | rs823123 | 11966 | 1 | 1 | PDGene |
| SNCA | rs356168 | rs356168 | 0 | 1 | 1 | PDGene |
| STK39 | rs10176669 | rs10208207 | 9887 | 0.824 | 0.936 | PDGene |
| | | rs2102808 | 32166 | 0.365 | 0.869 | PDGene |
| LAMP3 | rs9822789 | rs11711441 | 18401 | 0.948 | 1 | PDGene |
| HLA-DR | rs2076530 | rs3129882 | 45714 | 0.484 | 0.67 | T. R. |
| | | | | | | Hamza |
| | | | | | | et al. |

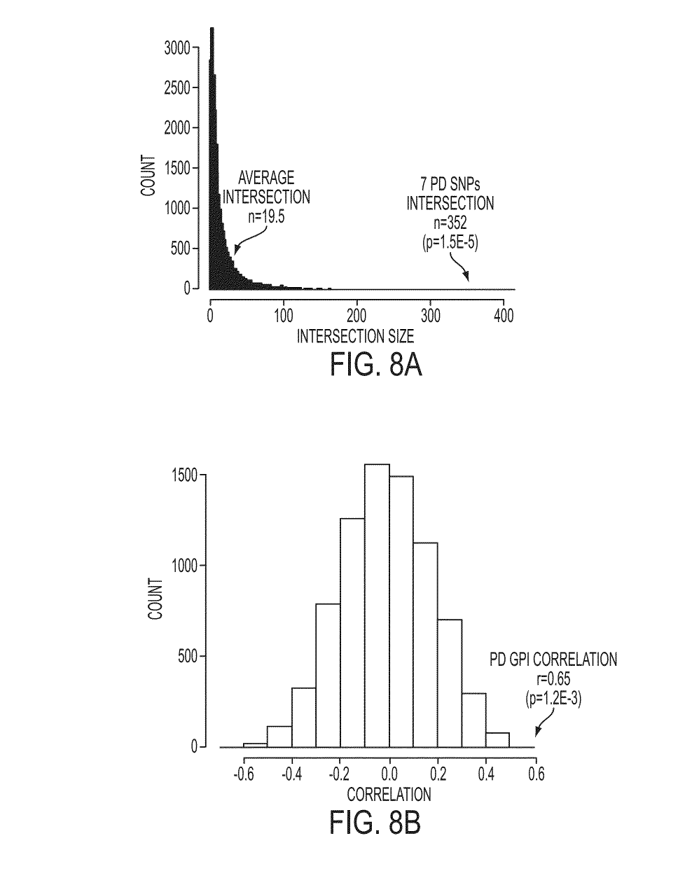

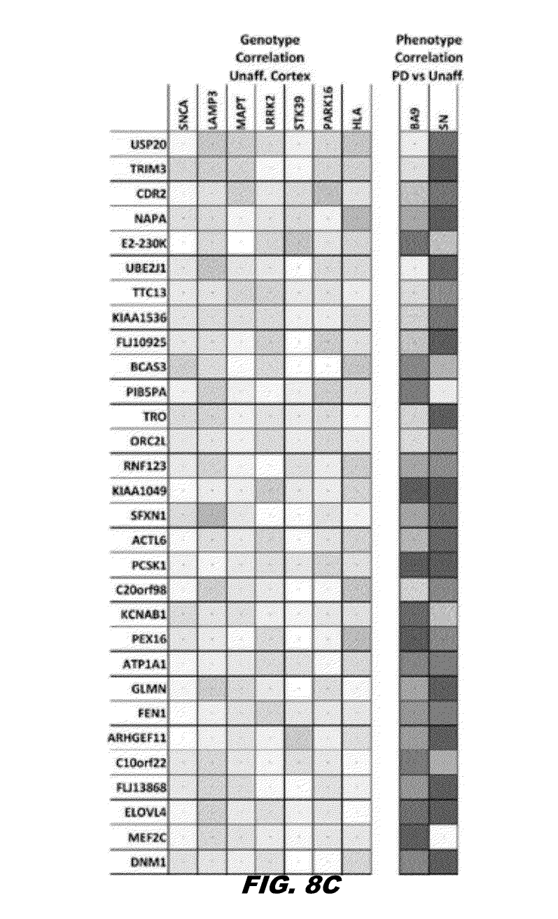

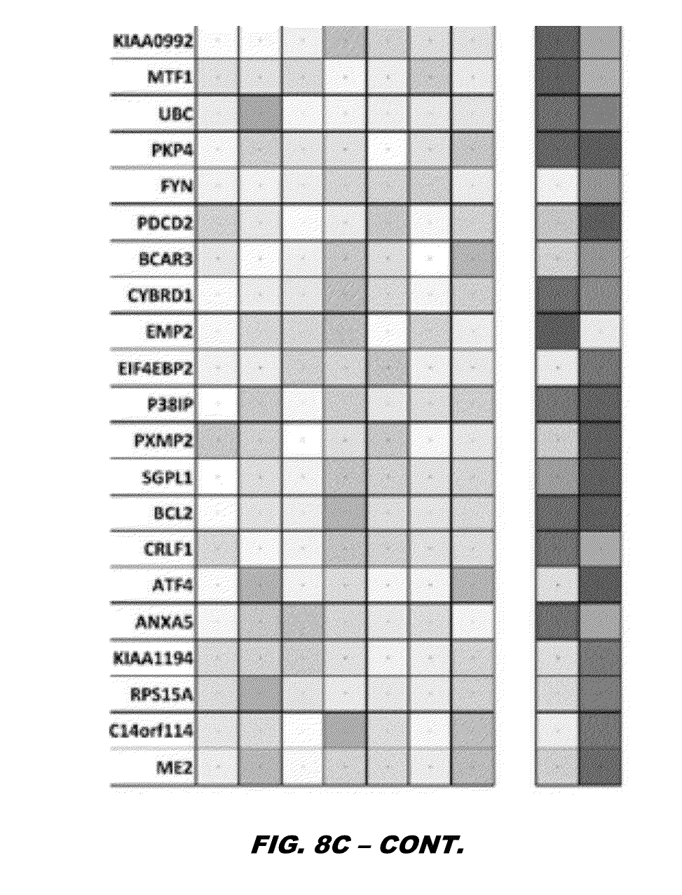

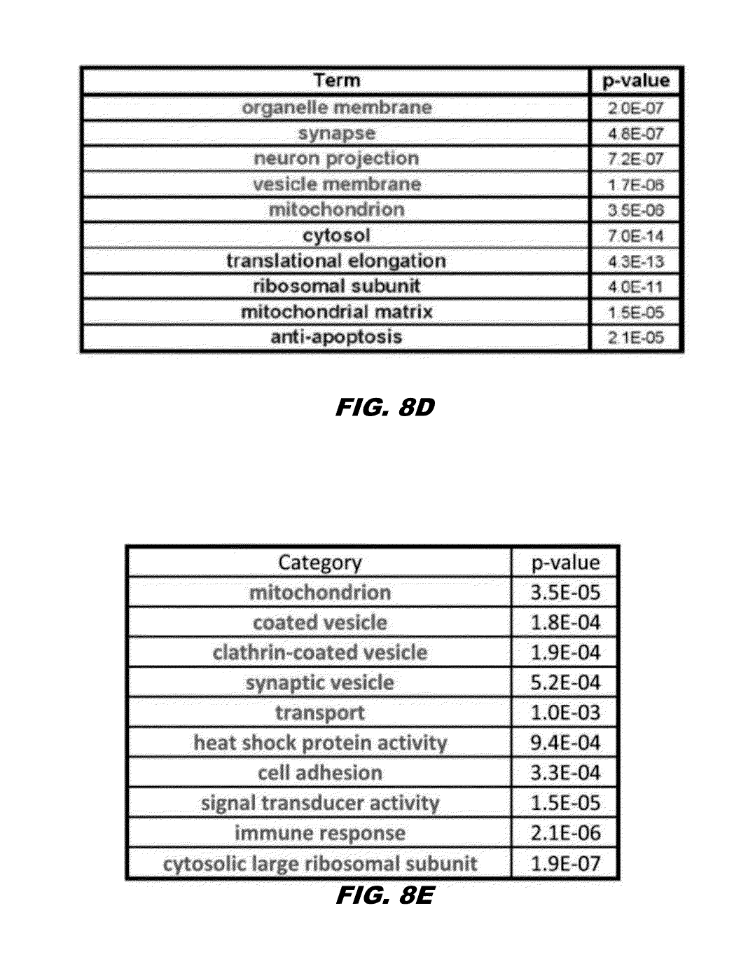

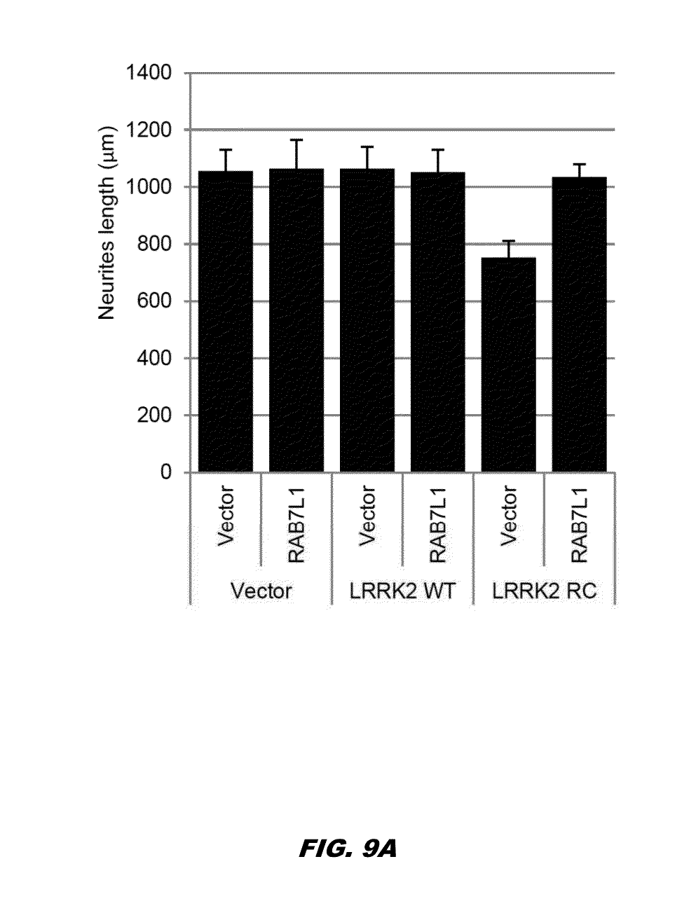

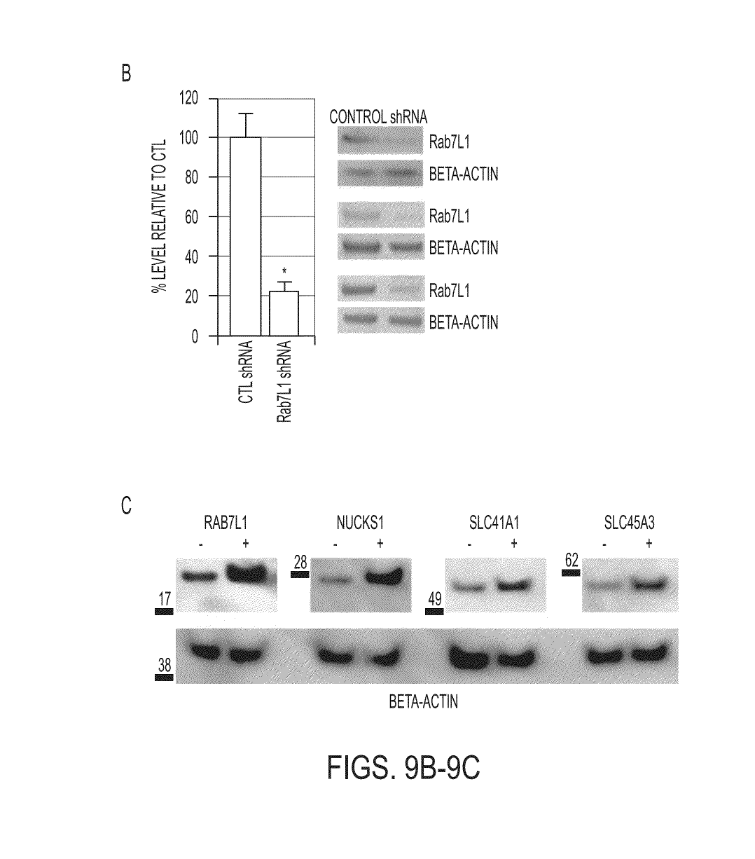

The GPIs of the 7 loci revealed a high degree of overlap in terms of the identity of transcripts altered in expression level and the valence of such alterations: genes were coordinately altered in their expression by each of the 7 PD-associated loci (over 15-fold greater than expected by chance: p=1.5E-5 by resampling statistics; FIGS. 8A-8B. Table 2).

TABLE 2
Individual gene transcripts commonly impacted by the high-risk
allele at 7 PD loci. Presented are the gene transcript-level GPI sub-components: the list of
genes whose expression levels correlate with the PD high-risk allele, and in the same
direction for each of the 7 PD loci studied (“SNCA”, “LRRK2”, “MAPT”, “PARK16”,
“HLA-DRA”, “STK39”, “LAMP3”). Genes are identified by their Illumina probesets
(“Probe”) and their Gene Symbol. Positive values correspond with a relative increase in gene
expression level in the presence of a high-risk allele, negative values with a decrease. The
average correlation across the 7 loci is indicated (“Average”) for each gene.

| | Gene | | | | | | | | |
|---|---|---|---|---|---|---|---|---|---|
| Probe | Symbol | SNCA | LRRK2 | MAPT | PARK16 | HLA-DRA | STK39 | LAMP3 | Avg. |
| GI_16945968-S | LRP15 | 0.10 | 0.17 | 0.09 | 0.17 | 0.00 | 0.16 | 0.14 | 0.12 |
| GI_42657118-S | LOC389203 | 0.04 | 0.17 | 0.13 | 0.09 | 0.07 | 0.17 | 0.11 | 0.11 |
| GI_30089996-A | BAF53A | 0.01 | 0.12 | 0.17 | 0.11 | 0.08 | 0.07 | 0.21 | 0.11 |
| GI_39777591-S | SLC2A10 | 0.09 | 0.25 | 0.11 | 0.01 | 0.10 | 0.16 | 0.03 | 0.11 |
| GI_16554595-A | IER3 | 0.03 | 0.14 | 0.10 | 0.11 | 0.20 | 0.02 | 0.14 | 0.11 |
| GI_38788371-S | AQR | 0.11 | 0.13 | 0.15 | 0.06 | 0.04 | 0.02 | 0.22 | 0.10 |
| GI_23238230-A | HMGN3 | 0.04 | 0.12 | 0.18 | 0.07 | 0.03 | 0.10 | 0.20 | 0.10 |
| GI_7705400-S | HDCMA18P | 0.05 | 0.17 | 0.21 | 0.06 | 0.08 | 0.06 | 0.12 | 0.10 |
| GI_4503932-S | GATM | 0.04 | 0.15 | 0.19 | 0.03 | 0.02 | 0.11 | 0.19 | 0.10 |
| GI_21450827-S | MGC7036 | 0.01 | 0.10 | 0.13 | 0.13 | 0.08 | 0.16 | 0.10 | 0.10 |
| GI_22748758-S | MGC40157 | 0.04 | 0.08 | 0.13 | 0.06 | 0.04 | 0.15 | 0.20 | 0.10 |
| GI_13376994-S | ME2 | 0.05 | 0.12 | 0.18 | 0.05 | 0.02 | 0.10 | 0.18 | 0.10 |
| GI_18860915-S | XRN2 | 0.08 | 0.13 | 0.14 | 0.00 | 0.07 | 0.08 | 0.20 | 0.10 |
| GI_8922630-S | C14orf114 | 0.07 | 0.22 | 0.17 | 0.03 | 0.01 | 0.10 | 0.10 | 0.10 |
| GI_34335150-S | RPS15A | 0.10 | 0.06 | 0.13 | 0.05 | 0.08 | 0.07 | 0.22 | 0.10 |
| GI_38502322-S | C9orf10OS | 0.04 | 0.17 | 0.16 | 0.05 | 0.00 | 0.05 | 0.22 | 0.10 |
| GI_42734361-S | DOCK7 | 0.10 | 0.10 | 0.18 | 0.05 | 0.01 | 0.18 | 0.06 | 0.10 |
| GI_6006027-S | NRAS | 0.05 | 0.19 | 0.12 | 0.04 | 0.13 | 0.07 | 0.08 | 0.10 |
| GI_4507668-S | TPT1 | 0.02 | 0.06 | 0.08 | 0.11 | 0.08 | 0.09 | 0.24 | 0.10 |
| GI_31543202-S | MGC8974 | 0.03 | 0.15 | 0.22 | 0.07 | 0.06 | 0.08 | 0.06 | 0.10 |
| GI_41393567-S | HEBP2 | 0.02 | 0.25 | 0.09 | 0.06 | 0.05 | 0.11 | 0.08 | 0.09 |
| GI_37551941-S | LOC284347 | 0.09 | 0.13 | 0.08 | 0.04 | 0.03 | 0.14 | 0.13 | 0.09 |
| GI_28872862-S | KIAA1194 | 0.10 | 0.08 | 0.12 | 0.05 | 0.13 | 0.04 | 0.12 | 0.09 |
| GI_4809273-S | ANXA5 | 0.03 | 0.11 | 0.03 | 0.10 | 0.16 | 0.06 | 0.15 | 0.09 |
| GI_23065549-S | GSTM2 | 0.02 | 0.07 | 0.17 | 0.10 | 0.07 | 0.11 | 0.11 | 0.09 |
| GI_4507894-S | VIM | 0.01 | 0.17 | 0.07 | 0.08 | 0.04 | 0.10 | 0.16 | 0.09 |
| GI_4557730-S | LTBP1 | 0.03 | 0.08 | 0.10 | 0.08 | 0.17 | 0.08 | 0.09 | 0.09 |
| GI_34222292-S | GYS1 | 0.01 | 0.10 | 0.10 | 0.17 | 0.14 | 0.07 | 0.05 | 0.09 |
| GI_37540659-S | KIAA1345 | 0.15 | 0.12 | 0.14 | 0.01 | 0.08 | 0.08 | 0.05 | 0.09 |
| GI_34485729-S | PRKY | 0.02 | 0.21 | 0.14 | 0.07 | 0.03 | 0.05 | 0.09 | 0.09 |
| GI_5031632-S | FARP1 | 0.04 | 0.07 | 0.16 | 0.13 | 0.11 | 0.04 | 0.07 | 0.09 |
| GI_33469973-A | ATF4 | 0.02 | 0.10 | 0.20 | 0.04 | 0.04 | 0.03 | 0.20 | 0.09 |
| GI_22095362-S | C14orf135 | 0.02 | 0.21 | 0.23 | 0.00 | 0.03 | 0.03 | 0.09 | 0.09 |
| GI_21361081-S | CRLF1 | 0.10 | 0.15 | 0.09 | 0.09 | 0.04 | 0.12 | 0.03 | 0.09 |
| GI_4557354-I | BCL2 | 0.01 | 0.20 | 0.10 | 0.08 | 0.05 | 0.09 | 0.08 | 0.09 |
| GI_45439305-S | DARS | 0.01 | 0.06 | 0.10 | 0.08 | 0.17 | 0.11 | 0.08 | 0.09 |
| GI_31982935-S | SGPL1 | 0.00 | 0.16 | 0.11 | 0.08 | 0.04 | 0.12 | 0.09 | 0.09 |
| GI_8923891-S | PXMP2 | 0.14 | 0.09 | 0.07 | 0.03 | 0.00 | 0.15 | 0.12 | 0.09 |
| GI_7661645-S | DKFZP566E144 | 0.08 | 0.13 | 0.13 | 0.02 | 0.10 | 0.03 | 0.12 | 0.09 |
| GI_4503182-S | CYB5 | 0.04 | 0.06 | 0.08 | 0.09 | 0.01 | 0.06 | 0.25 | 0.09 |
| GI_15431291-S | RPL12 | 0.01 | 0.09 | 0.08 | 0.04 | 0.01 | 0.13 | 0.24 | 0.09 |
| GI_40255312-S | P38IP | 0.00 | 0.13 | 0.13 | 0.09 | 0.03 | 0.07 | 0.14 | 0.09 |
| GI_31542585-S | EIF4EBP2 | 0.04 | 0.11 | 0.06 | 0.04 | 0.12 | 0.16 | 0.05 | 0.08 |
| GI_31343475-S | GNA13 | 0.00 | 0.12 | 0.14 | 0.07 | 0.04 | 0.10 | 0.10 | 0.08 |
| GI_24475891-S | CSPG6 | 0.01 | 0.08 | 0.17 | 0.04 | 0.01 | 0.05 | 0.24 | 0.08 |
| GI_42716292-S | EMP2 | 0.02 | 0.16 | 0.05 | 0.12 | 0.11 | 0.02 | 0.11 | 0.08 |
| GI_31543652-S | SRP14 | 0.01 | 0.17 | 0.08 | 0.04 | 0.04 | 0.11 | 0.13 | 0.08 |
| GI_25453469-S | EEF1A1 | 0.04 | 0.08 | 0.08 | 0.07 | 0.02 | 0.09 | 0.19 | 0.08 |
| GI_27436919-I | SPAG9 | 0.03 | 0.11 | 0.13 | 0.04 | 0.07 | 0.06 | 0.14 | 0.08 |
| GI_21359839-S | SNRPG | 0.01 | 0.06 | 0.10 | 0.08 | 0.01 | 0.12 | 0.19 | 0.08 |
| GI_15451787-S | PDGFRA | 0.00 | 0.11 | 0.06 | 0.13 | 0.02 | 0.16 | 0.10 | 0.08 |
| GI_19923602-S | CYBRD1 | 0.01 | 0.17 | 0.13 | 0.03 | 0.07 | 0.10 | 0.05 | 0.08 |
| GI_19743895-A | TADA3L | 0.10 | 0.04 | 0.11 | 0.06 | 0.01 | 0.12 | 0.12 | 0.08 |
| GI_5730084-S | TCTEL1 | 0.04 | 0.10 | 0.07 | 0.13 | 0.04 | 0.11 | 0.08 | 0.08 |
| GI_15193293-S | PGR1 | 0.13 | 0.00 | 0.12 | 0.07 | 0.01 | 0.02 | 0.22 | 0.08 |
| GI_21704284-S | JAM2 | 0.07 | 0.09 | 0.10 | 0.07 | 0.10 | 0.03 | 0.09 | 0.08 |
| GI_4502370-S | BCAR3 | 0.06 | 0.15 | 0.19 | 0.00 | 0.05 | 0.09 | 0.01 | 0.08 |
| GI_37541013-S | LOC374395 | 0.01 | 0.00 | 0.08 | 0.08 | 0.08 | 0.10 | 0.20 | 0.08 |
| GI_34304355-A | SCAPIN1 | 0.02 | 0.13 | 0.10 | 0.13 | 0.07 | 0.04 | 0.07 | 0.08 |
| GI_21735593-I | PDCD2 | 0.12 | 0.06 | 0.12 | 0.03 | 0.01 | 0.14 | 0.07 | 0.08 |
| GI_23510344-I | FYN | 0.04 | 0.11 | 0.05 | 0.13 | 0.05 | 0.12 | 0.04 | 0.08 |
| GI_7661537-S | BRI3 | 0.12 | 0.14 | 0.07 | 0.06 | 0.05 | 0.03 | 0.08 | 0.08 |
| GI_6806894-S | PKP4 | 0.02 | 0.08 | 0.15 | 0.08 | 0.07 | 0.01 | 0.13 | 0.08 |
| GI_41872597-S | CPNE3 | 0.00 | 0.19 | 0.17 | 0.08 | 0.00 | 0.10 | 0.01 | 0.08 |
| GI_41350211-S | BRD7 | 0.00 | 0.07 | 0.11 | 0.05 | 0.00 | 0.08 | 0.22 | 0.08 |
| GI_37542859-S | DKFZp313M0720 | 0.06 | 0.13 | 0.07 | 0.08 | 0.03 | 0.08 | 0.10 | 0.08 |
| GI_34304116-S | UBC | 0.05 | 0.03 | 0.07 | 0.06 | 0.03 | 0.05 | 0.23 | 0.08 |
| GI_5174588-S | MTF1 | 0.08 | 0.02 | 0.06 | 0.14 | 0.10 | 0.05 | 0.08 | 0.08 |
| GI_21361584-S | KIAA0992 | 0.01 | 0.17 | 0.08 | 0.09 | 0.04 | 0.12 | 0.01 | 0.08 |
| GI_4503532-S | EIF4B | 0.03 | 0.09 | 0.10 | 0.11 | 0.02 | 0.01 | 0.17 | 0.08 |
| GI_19263339-S | GPT2 | 0.04 | 0.13 | 0.04 | 0.08 | 0.07 | 0.14 | 0.01 | 0.07 |
| GI_40255140-S | ChGn | 0.20 | 0.10 | 0.06 | 0.01 | 0.03 | 0.10 | 0.02 | 0.07 |
| GI_41406065-A | H2AV | 0.01 | 0.03 | 0.10 | 0.21 | 0.03 | 0.09 | 0.04 | 0.07 |
| GI_30795205-S | PPP2R5A | 0.05 | 0.07 | 0.07 | 0.08 | 0.08 | 0.08 | 0.08 | 0.07 |
| GI_31542744-S | FLJ23091 | 0.06 | 0.14 | 0.04 | 0.09 | 0.02 | 0.12 | 0.03 | 0.07 |
| GI_22538424-S | ATPAF2 | 0.09 | 0.08 | 0.08 | 0.12 | 0.03 | 0.08 | 0.02 | 0.07 |
| GI_24308042-S | KIAA0828 | 0.04 | 0.08 | 0.09 | 0.07 | 0.08 | 0.11 | 0.02 | 0.07 |
| GI_28316809-S | MGC31967 | 0.02 | 0.07 | 0.08 | 0.06 | 0.06 | 0.02 | 0.19 | 0.07 |
| GI_16905527-A | DAP3 | 0.03 | 0.10 | 0.07 | 0.09 | 0.01 | 0.04 | 0.17 | 0.07 |
| GI_22035601-A | MAP4K4 | 0.01 | 0.07 | 0.06 | 0.14 | 0.08 | 0.02 | 0.12 | 0.07 |
| GI_24497491-S | SLC22A5 | 0.02 | 0.09 | 0.12 | 0.11 | 0.09 | 0.04 | 0.03 | 0.07 |
| GI_21536323-A | HNRPUL1 | 0.02 | 0.04 | 0.03 | 0.05 | 0.14 | 0.09 | 0.13 | 0.07 |
| GI_40254815-S | HSPCA | 0.04 | 0.09 | 0.14 | 0.01 | 0.00 | 0.04 | 0.18 | 0.07 |
| GI_32481212-S | MK-STYX | 0.05 | 0.03 | 0.15 | 0.02 | 0.05 | 0.05 | 0.16 | 0.07 |
| GI_17402905-S | RPL22 | 0.04 | 0.03 | 0.05 | 0.02 | 0.05 | 0.13 | 0.17 | 0.07 |
| GI_28466988-S | ATP10D | 0.08 | 0.15 | 0.10 | 0.05 | 0.05 | 0.07 | 0.00 | 0.07 |
| GI_10346134-S | MAPRE2 | 0.03 | 0.04 | 0.13 | 0.04 | 0.06 | 0.03 | 0.16 | 0.07 |
| GI_33946332-I | ZC3HAV1 | 0.05 | 0.19 | 0.02 | 0.03 | 0.07 | 0.10 | 0.04 | 0.07 |
| GI_41393560-S | LAP3 | 0.03 | 0.07 | 0.08 | 0.11 | 0.03 | 0.05 | 0.11 | 0.07 |
| GI_10863994-S | ZNF410 | 0.03 | 0.11 | 0.10 | 0.07 | 0.07 | 0.05 | 0.08 | 0.07 |
| GI_6715608-S | MAPK4 | 0.07 | 0.12 | 0.07 | 0.08 | 0.03 | 0.06 | 0.05 | 0.07 |
| GI_4505336-S | NUBP1 | 0.06 | 0.09 | 0.04 | 0.06 | 0.10 | 0.05 | 0.08 | 0.07 |
| GI_40353732-S | NPM1 | 0.01 | 0.05 | 0.13 | 0.01 | 0.05 | 0.03 | 0.19 | 0.07 |
| GI_5579480-S | ARHN | 0.06 | 0.05 | 0.09 | 0.01 | 0.05 | 0.00 | 0.21 | 0.07 |
| GI_4503606-S | ETFA | 0.04 | 0.06 | 0.09 | 0.06 | 0.01 | 0.15 | 0.05 | 0.07 |
| GI_34147357-S | MGC2747 | 0.07 | 0.03 | 0.01 | 0.04 | 0.11 | 0.11 | 0.11 | 0.07 |
| GI_4503174-S | CXCR4 | 0.05 | 0.06 | 0.12 | 0.08 | 0.08 | 0.02 | 0.06 | 0.07 |
| GI_37552472-S | LOC286088 | 0.02 | 0.04 | 0.10 | 0.06 | 0.03 | 0.00 | 0.22 | 0.07 |
| GI_24432092-S | PHF13 | 0.01 | 0.08 | 0.12 | 0.02 | 0.07 | 0.09 | 0.08 | 0.07 |
| GI_38683837-S | CD47 | 0.01 | 0.06 | 0.12 | 0.11 | 0.04 | 0.02 | 0.11 | 0.07 |
| GI_31543149-S | MGC11308 | 0.01 | 0.05 | 0.10 | 0.13 | 0.06 | 0.02 | 0.09 | 0.07 |
| GI_4501882-S | ACTA2 | 0.02 | 0.05 | 0.01 | 0.11 | 0.16 | 0.06 | 0.05 | 0.07 |
| GI_4557394-S | CA2 | 0.04 | 0.10 | 0.10 | 0.01 | 0.03 | 0.04 | 0.14 | 0.07 |
| GI_28872718-S | BTG2 | 0.04 | 0.08 | 0.01 | 0.12 | 0.22 | 0.00 | 0.00 | 0.07 |
| GI_45359860-S | POLR2L | 0.05 | 0.03 | 0.03 | 0.09 | 0.00 | 0.14 | 0.12 | 0.07 |
| GI_17864091-S | DNAH7 | 0.06 | 0.08 | 0.11 | 0.07 | 0.05 | 0.08 | 0.01 | 0.07 |
| GI_20986484-S | YAP1 | 0.01 | 0.15 | 0.06 | 0.03 | 0.03 | 0.14 | 0.04 | 0.07 |
| GI_34147334-S | FLJ20811 | 0.06 | 0.00 | 0.06 | 0.02 | 0.05 | 0.07 | 0.19 | 0.07 |
| GI_22538460-S | NCOR1 | 0.05 | 0.09 | 0.08 | 0.00 | 0.01 | 0.01 | 0.22 | 0.07 |
| GI_15451934-I | CDC14B | 0.02 | 0.13 | 0.07 | 0.06 | 0.07 | 0.04 | 0.06 | 0.07 |
| GI_4557378-S | SERPING1 | 0.07 | 0.07 | 0.00 | 0.08 | 0.11 | 0.06 | 0.07 | 0.06 |
| GI_29789284-S | COMMD7 | 0.07 | 0.00 | 0.11 | 0.10 | 0.12 | 0.04 | 0.01 | 0.06 |
| GI_4506342-S | PXMP3 | 0.02 | 0.14 | 0.10 | 0.03 | 0.01 | 0.11 | 0.03 | 0.06 |
| GI_24308106-S | DKFZp566C0424 | 0.00 | 0.12 | 0.03 | 0.07 | 0.11 | 0.05 | 0.07 | 0.06 |
| GI_34328906-I | DNAJB6 | 0.04 | 0.07 | 0.10 | 0.07 | 0.06 | 0.03 | 0.08 | 0.06 |
| GI_38373686-S | AP1G1 | 0.05 | 0.02 | 0.12 | 0.06 | 0.08 | 0.07 | 0.05 | 0.06 |
| GI_13654234-A | RGS20 | 0.06 | 0.11 | 0.05 | 0.05 | 0.02 | 0.14 | 0.02 | 0.06 |
| GI_4502100-S | ANXA1 | 0.03 | 0.07 | 0.02 | 0.08 | 0.08 | 0.01 | 0.14 | 0.06 |
| GI_34222274-S | SSA2 | 0.05 | 0.11 | 0.08 | 0.06 | 0.11 | 0.01 | 0.01 | 0.06 |
| GI_15011920-S | NOLA3 | 0.12 | 0.02 | 0.06 | 0.05 | 0.04 | 0.06 | 0.08 | 0.06 |
| GI_23510452-S | COTL1 | 0.03 | 0.06 | 0.02 | 0.01 | 0.10 | 0.10 | 0.10 | 0.06 |
| GI_23111020-A | RGN | 0.01 | 0.10 | 0.08 | 0.01 | 0.01 | 0.17 | 0.05 | 0.06 |
| GI_32483398-S | PAK2 | 0.07 | 0.04 | 0.13 | 0.12 | 0.01 | 0.03 | 0.04 | 0.06 |
| GI_10835186-S | SOD2 | 0.06 | 0.02 | 0.03 | 0.16 | 0.05 | 0.06 | 0.05 | 0.06 |
| GI_23346408-S | C20orf111 | 0.06 | 0.02 | 0.07 | 0.06 | 0.03 | 0.06 | 0.12 | 0.06 |
| GI_5032214-S | UK114 | 0.01 | 0.09 | 0.10 | 0.04 | 0.01 | 0.15 | 0.03 | 0.06 |
| GI_39725690-S | SPUF | 0.02 | 0.13 | 0.05 | 0.05 | 0.05 | 0.02 | 0.11 | 0.06 |
| GI_40255040-S | TMP21 | 0.01 | 0.04 | 0.09 | 0.02 | 0.01 | 0.07 | 0.18 | 0.06 |
| GI_39725692-S | FLJ10420 | 0.01 | 0.07 | 0.03 | 0.05 | 0.07 | 0.08 | 0.12 | 0.06 |
| GI_31542848-S | GMPR | 0.11 | 0.05 | 0.04 | 0.08 | 0.08 | 0.01 | 0.05 | 0.06 |
| GI_24475893-S | GNB2L1 | 0.02 | 0.03 | 0.02 | 0.05 | 0.02 | 0.08 | 0.19 | 0.06 |
| GI_31581523-S | COBL | 0.02 | 0.15 | 0.11 | 0.06 | 0.02 | 0.03 | 0.03 | 0.06 |
| GI_18375506-S | APEX2 | 0.01 | 0.11 | 0.06 | 0.01 | 0.10 | 0.11 | 0.02 | 0.06 |
| GI_34147490-S | APG3 | 0.05 | 0.01 | 0.11 | 0.00 | 0.02 | 0.04 | 0.18 | 0.06 |
| GI_39930611-I | KLHL5 | 0.01 | 0.10 | 0.07 | 0.08 | 0.10 | 0.05 | 0.01 | 0.06 |
| GI_42658769-S | LOC401457 | 0.09 | 0.08 | 0.03 | 0.03 | 0.05 | 0.00 | 0.13 | 0.06 |
| GI_40254977-S | FIP1L1 | 0.01 | 0.06 | 0.05 | 0.06 | 0.10 | 0.03 | 0.10 | 0.06 |
| GI_23510357-A | RIOK1 | 0.04 | 0.06 | 0.14 | 0.05 | 0.02 | 0.02 | 0.07 | 0.06 |
| GI_33946290-S | FLJ12443 | 0.00 | 0.03 | 0.03 | 0.19 | 0.03 | 0.09 | 0.03 | 0.06 |
| GI_21361092-S | TPST1 | 0.15 | 0.05 | 0.00 | 0.02 | 0.01 | 0.08 | 0.10 | 0.06 |
| GI_40254896-S | DKFZp434K1210 | 0.05 | 0.10 | 0.02 | 0.08 | 0.05 | 0.05 | 0.05 | 0.06 |
| GI_8922853-S | FLJ11078 | 0.07 | 0.10 | 0.04 | 0.06 | 0.04 | 0.07 | 0.01 | 0.06 |
| GI_8051633-S | RARRES3 | 0.05 | 0.03 | 0.02 | 0.12 | 0.03 | 0.08 | 0.08 | 0.06 |
| GI_31543826-I | TSC22 | 0.03 | 0.09 | 0.12 | 0.01 | 0.03 | 0.00 | 0.12 | 0.06 |
| GI_37694061-I | AQP1 | 0.04 | 0.06 | 0.02 | 0.11 | 0.03 | 0.08 | 0.06 | 0.06 |
| GI_19747274-A | PHF10 | 0.02 | 0.16 | 0.03 | 0.07 | 0.08 | 0.02 | 0.01 | 0.06 |
| GI_4557342-S | ALDH7A1 | 0.02 | 0.06 | 0.04 | 0.09 | 0.01 | 0.15 | 0.02 | 0.06 |
| GI_14043023-S | BAG3 | 0.01 | 0.13 | 0.01 | 0.07 | 0.14 | 0.02 | 0.02 | 0.06 |
| GI_18105013-A | 3-Apr | 0.00 | 0.04 | 0.02 | 0.03 | 0.07 | 0.10 | 0.13 | 0.06 |
| GI_21618360-S | FXYD5 | 0.04 | 0.06 | 0.02 | 0.08 | 0.09 | 0.02 | 0.09 | 0.06 |
| GI_4503100-S | CSRP2 | 0.03 | 0.09 | 0.06 | 0.05 | 0.07 | 0.02 | 0.08 | 0.06 |
| GI_23308566-S | ASRGL1 | 0.05 | 0.10 | 0.01 | 0.11 | 0.02 | 0.08 | 0.02 | 0.06 |
| GI_42658538-S | DKFZP434A0225 | 0.03 | 0.03 | 0.06 | 0.13 | 0.09 | 0.02 | 0.03 | 0.06 |
| GI_40255086-S | LOC118491 | 0.04 | 0.05 | 0.10 | 0.02 | 0.07 | 0.06 | 0.06 | 0.06 |
| GI_7661691-S | DKFZP586N0721 | 0.09 | 0.13 | 0.00 | 0.07 | 0.02 | 0.01 | 0.05 | 0.06 |
| GI_4757755-S | ANXA2 | 0.01 | 0.07 | 0.04 | 0.08 | 0.10 | 0.01 | 0.06 | 0.05 |
| GI_14249551-S | DIRC2 | 0.06 | 0.09 | 0.05 | 0.05 | 0.08 | 0.02 | 0.04 | 0.05 |
| GI_20070179-S | EIF4EBP1 | 0.02 | 0.07 | 0.05 | 0.14 | 0.04 | 0.01 | 0.05 | 0.05 |
| GI_4503680-S | FCGBP | 0.01 | 0.06 | 0.05 | 0.03 | 0.08 | 0.03 | 0.12 | 0.05 |
| GI_41188450-S | LOC388727 | 0.01 | 0.04 | 0.01 | 0.15 | 0.08 | 0.08 | 0.01 | 0.05 |
| GI_42717993-A | DTNA | 0.04 | 0.08 | 0.06 | 0.06 | 0.02 | 0.05 | 0.08 | 0.05 |
| GI_4505488-S | ODC1 | 0.10 | 0.10 | 0.03 | 0.00 | 0.04 | 0.05 | 0.05 | 0.05 |
| GI_19923436-S | AK3L1 | 0.04 | 0.06 | 0.01 | 0.02 | 0.10 | 0.13 | 0.00 | 0.05 |
| GI_31341159-S | MGC21416 | 0.15 | 0.02 | 0.06 | 0.00 | 0.11 | 0.01 | 0.00 | 0.05 |
| GI_8922937-S | FLJ11200 | 0.00 | 0.07 | 0.10 | 0.05 | 0.05 | 0.06 | 0.03 | 0.05 |
| GI_34222296-S | FCGRT | 0.01 | 0.08 | 0.02 | 0.07 | 0.07 | 0.09 | 0.02 | 0.05 |
| GI_32307151-S | OXTR | 0.11 | 0.04 | 0.02 | 0.04 | 0.03 | 0.04 | 0.07 | 0.05 |
| GI_34222132-S | TXNDC | 0.04 | 0.07 | 0.06 | 0.04 | 0.04 | 0.09 | 0.02 | 0.05 |
| GI_22035637-A | MGST1 | 0.01 | 0.09 | 0.01 | 0.06 | 0.02 | 0.13 | 0.03 | 0.05 |
| GI_34147669-S | SLC39A1 | 0.02 | 0.04 | 0.04 | 0.04 | 0.13 | 0.02 | 0.04 | 0.05 |
| GI_21071079-S | FBXL7 | 0.00 | 0.09 | 0.03 | 0.06 | 0.06 | 0.08 | 0.01 | 0.05 |
| GI_31543361-S | ORF1-FL49 | 0.05 | 0.08 | 0.02 | 0.07 | 0.01 | 0.10 | 0.00 | 0.05 |
| GI_42716312-S | ANG | 0.01 | 0.02 | 0.05 | 0.06 | 0.08 | 0.09 | 0.02 | 0.05 |
| GI_6006016-S | LGALS3BP | 0.01 | 0.05 | 0.03 | 0.10 | 0.06 | 0.02 | 0.06 | 0.05 |
| GI_40549455-S | NHS | 0.01 | 0.13 | 0.05 | 0.03 | 0.04 | 0.04 | 0.03 | 0.05 |
| GI_7019466-S | CNOT4 | 0.02 | 0.12 | 0.09 | 0.05 | 0.00 | 0.03 | 0.02 | 0.05 |
| GI_24797094-A | PYCR1 | 0.01 | 0.13 | 0.06 | 0.07 | 0.03 | 0.01 | 0.02 | 0.05 |
| GI_7657057-S | EIF2B2 | 0.02 | 0.06 | 0.05 | 0.02 | 0.03 | 0.10 | 0.05 | 0.05 |
| GI_20127485-S | M6PRBP1 | 0.00 | 0.01 | 0.00 | 0.10 | 0.11 | 0.04 | 0.06 | 0.05 |
| GI_4755145-S | AEBP1 | 0.01 | 0.02 | 0.02 | 0.12 | 0.08 | 0.01 | 0.05 | 0.04 |
| GI_33943097-S | RAB5B | 0.07 | 0.00 | 0.06 | 0.07 | 0.09 | 0.01 | 0.00 | 0.04 |
| GI_16445423-S | WDR12 | 0.04 | 0.08 | 0.04 | 0.02 | 0.02 | 0.02 | 0.08 | 0.04 |
| GI_42476129-S | RAP80 | 0.04 | 0.03 | 0.07 | 0.07 | 0.06 | 0.03 | 0.01 | 0.04 |
| GI_34147349-S | MGC2601 | 0.03 | 0.03 | 0.03 | 0.03 | 0.08 | 0.08 | 0.02 | 0.04 |
| GI_29789057-S | KIBRA | 0.04 | 0.06 | 0.01 | 0.00 | 0.04 | 0.11 | 0.03 | 0.04 |
| GI_14670391-A | BAZ1B | 0.04 | 0.00 | 0.07 | 0.00 | 0.12 | 0.01 | 0.04 | 0.04 |
| GI_4557800-S | NP | 0.04 | 0.01 | 0.02 | 0.10 | 0.02 | 0.06 | 0.03 | 0.04 |
| GI_34147469-S | MGC15396 | 0.02 | 0.08 | 0.03 | 0.03 | 0.07 | 0.02 | 0.05 | 0.04 |
| GI_13129101-S | MGC955 | 0.04 | 0.00 | 0.02 | 0.10 | 0.02 | 0.06 | 0.03 | 0.04 |
| GI_39753966-S | CSPG5 | 0.00 | 0.02 | 0.04 | 0.03 | 0.00 | 0.08 | 0.11 | 0.04 |
| GI_33636718-S | TIMM44 | 0.04 | 0.03 | 0.05 | 0.02 | 0.07 | 0.04 | 0.02 | 0.04 |
| GI_40354215-S | SIX5 | 0.00 | 0.10 | 0.02 | 0.07 | 0.04 | 0.05 | 0.00 | 0.04 |
| GI_18702322-S | DNCL2B | 0.02 | 0.10 | 0.06 | 0.02 | 0.04 | 0.02 | 0.01 | 0.04 |
| GI_20127480-S | EPM2A | 0.08 | 0.02 | 0.00 | 0.04 | 0.06 | 0.05 | 0.02 | 0.04 |
| GI_18104963-A | CAPS | 0.00 | 0.05 | 0.01 | 0.05 | 0.10 | 0.03 | 0.02 | 0.04 |
| GI_4507432-S | TEGT | 0.04 | 0.04 | 0.01 | 0.04 | 0.08 | 0.06 | 0.00 | 0.04 |
| GI_24586683-A | DMN | 0.02 | 0.07 | 0.06 | 0.00 | 0.07 | 0.02 | 0.02 | 0.04 |
| GI_20149303-S | KIAA1160 | 0.09 | 0.03 | 0.03 | 0.03 | 0.00 | 0.02 | 0.05 | 0.04 |
| GI_4557586-S | FAH | 0.06 | 0.01 | 0.03 | 0.05 | 0.04 | 0.07 | 0.01 | 0.04 |
| GI_11496992-S | ADPRTL3 | 0.07 | 0.06 | 0.03 | 0.04 | 0.04 | 0.01 | 0.00 | 0.04 |
| GI_37550187-S | LOC375468 | 0.01 | 0.12 | 0.01 | 0.02 | 0.00 | 0.01 | 0.07 | 0.04 |
| GI_34222312-S | GPSM2 | 0.01 | 0.09 | 0.02 | 0.04 | 0.01 | 0.01 | 0.05 | 0.03 |
| GI_34147344-S | KCTD14 | 0.04 | 0.08 | 0.08 | 0.03 | 0.00 | 0.00 | 0.01 | 0.03 |
| GI_7706271-S | CGI-30 | 0.07 | 0.06 | 0.02 | 0.00 | 0.01 | 0.07 | 0.01 | 0.03 |
| GI_34147623-S | SORD | 0.02 | 0.04 | 0.09 | 0.02 | 0.02 | 0.04 | 0.01 | 0.03 |
| GI_40255159-S | MGC20446 | 0.04 | 0.06 | 0.06 | 0.01 | 0.00 | 0.04 | 0.01 | 0.03 |
| GI_19311011-S | BRIX | 0.00 | 0.04 | 0.04 | 0.01 | 0.01 | 0.02 | 0.10 | 0.03 |
| GI_24797087-A | PEX10 | 0.01 | 0.02 | 0.00 | 0.00 | 0.02 | 0.12 | 0.05 | 0.03 |
| GI_20336263-A | GGA2 | 0.06 | 0.03 | 0.01 | 0.02 | 0.02 | 0.06 | 0.02 | 0.03 |
| GI_21361710-S | HCNGP | 0.02 | 0.00 | 0.02 | 0.00 | 0.02 | 0.04 | 0.03 | 0.02 |
| GI_31543911-S | USP20 | −0.02 | −0.11 | −0.16 | −0.12 | −0.14 | −0.04 | −0.16 | −0.11 |
| GI_5902039-S | RABL2B | −0.01 | −0.04 | −0.05 | −0.25 | −0.15 | −0.06 | −0.14 | −0.10 |
| GI_32454736-A | TRIM3 | −0.12 | −0.02 | −0.13 | −0.12 | −0.13 | −0.02 | −0.14 | −0.10 |
| GI_18598508-S | CDR2 | −0.02 | −0.07 | −0.09 | −0.18 | −0.14 | −0.10 | −0.08 | −0.10 |
| GI_7549818-A | RABL2A | −0.04 | −0.02 | −0.05 | −0.23 | −0.16 | −0.05 | −0.13 | −0.10 |
| GI_14149994-S | DKFZp434N035 | −0.02 | −0.13 | −0.18 | −0.07 | −0.11 | −0.06 | −0.11 | −0.10 |
| GI_37577147-A | NCKIPSD | −0.07 | −0.06 | −0.11 | −0.11 | −0.11 | −0.04 | −0.17 | −0.10 |
| GI_34147578-S | PISD | −0.06 | −0.02 | −0.14 | −0.14 | −0.13 | −0.08 | −0.09 | −0.10 |
| GI_4505328-S | NAPA | −0.09 | −0.07 | −0.21 | −0.04 | −0.03 | −0.10 | −0.11 | −0.09 |
| GI_13162281-S | STS | −0.02 | −0.15 | −0.10 | −0.13 | −0.04 | −0.09 | −0.11 | −0.09 |
| GI_21450766-S | C6orf136 | −0.05 | −0.12 | −0.10 | −0.10 | −0.10 | −0.07 | −0.09 | −0.09 |
| GI_11641403-S | CKMT1 | −0.05 | −0.17 | −0.08 | −0.08 | −0.10 | −0.07 | −0.06 | −0.09 |
| GI_33636749-S | E2-230K | 0.00 | −0.12 | −0.12 | −0.08 | 0.00 | −0.16 | −0.11 | −0.09 |
| GI_37577121-I | UBE2J1 | −0.07 | −0.07 | −0.11 | −0.11 | −0.07 | −0.01 | −0.17 | −0.09 |
| GI_31377702-S | TTC13 | −0.06 | −0.13 | −0.05 | −0.09 | −0.12 | −0.06 | −0.08 | −0.09 |
| GI_32528302-S | INSM2 | −0.05 | −0.07 | −0.18 | −0.17 | −0.02 | −0.02 | −0.09 | −0.08 |
| GI_14149741-S | KIAA1536 | −0.08 | −0.11 | −0.10 | −0.06 | −0.09 | −0.04 | −0.10 | −0.08 |
| GI_34222158-S | FLJ10925 | −0.07 | −0.14 | −0.09 | −0.15 | −0.03 | 0.00 | −0.09 | −0.08 |
| GI_8923764-S | CACNA2D3 | −0.01 | −0.17 | −0.06 | −0.04 | −0.05 | −0.08 | −0.15 | −0.08 |
| GI_28372510-S | ZDHHC22 | −0.02 | −0.14 | −0.12 | −0.14 | −0.11 | −0.03 | 0.00 | −0.08 |
| GI_21735551-S | MAP3K12 | −0.08 | −0.12 | −0.12 | −0.02 | −0.03 | −0.01 | −0.16 | −0.08 |
| GI_21361926-S | C6orf31 | −0.06 | −0.03 | −0.11 | −0.05 | −0.14 | −0.05 | −0.11 | −0.08 |
| GI_22095352-S | BCAS3 | −0.14 | −0.11 | −0.17 | 0.00 | −0.01 | 0.00 | −0.10 | −0.08 |
| GI_24430154-A | PSMC4 | −0.06 | −0.10 | −0.14 | −0.08 | −0.10 | 0.00 | −0.06 | −0.08 |
| GI_24307956-S | PIB5PA | −0.04 | −0.03 | −0.09 | −0.14 | −0.04 | −0.05 | −0.14 | −0.08 |
| GI_29540546-A | TRO | −0.10 | −0.08 | −0.03 | −0.10 | −0.04 | −0.05 | −0.13 | −0.08 |
| GI_32454751-S | ORC2L | −0.07 | −0.11 | −0.06 | −0.12 | −0.04 | −0.05 | −0.08 | −0.08 |
| GI_37588868-S | RNF123 | −0.06 | −0.01 | −0.15 | −0.06 | −0.03 | −0.09 | −0.13 | −0.08 |
| GI_22538494-S | WBSCR17 | −0.07 | −0.09 | −0.12 | −0.01 | −0.11 | −0.06 | −0.07 | −0.08 |
| GI_14149656-S | KIAA1049 | −0.01 | −0.15 | −0.13 | −0.06 | −0.05 | −0.06 | −0.06 | −0.07 |
| GI_23618866-S | SFXN1 | −0.10 | −0.01 | −0.04 | −0.08 | −0.07 | −0.01 | −0.22 | −0.07 |
| GI_31342415-S | LOC90529 | −0.01 | −0.06 | −0.08 | −0.09 | −0.09 | −0.08 | −0.12 | −0.07 |
| GI_17017983-S | CDK9 | −0.10 | −0.10 | −0.05 | −0.05 | −0.04 | −0.03 | −0.15 | −0.07 |
| GI_21536352-S | ACTL6 | −0.01 | −0.12 | −0.13 | −0.08 | −0.06 | −0.01 | −0.09 | −0.07 |
| GI_38158008-A | CIDEA | −0.01 | −0.17 | −0.02 | −0.05 | −0.10 | −0.04 | −0.13 | −0.07 |
| GI_20336241-S | PCSK1 | −0.04 | −0.09 | −0.09 | −0.12 | −0.07 | −0.07 | −0.03 | −0.07 |
| GI_19557635-A | PPIL3 | −0.02 | −0.01 | −0.11 | −0.12 | −0.01 | −0.11 | −0.13 | −0.07 |
| GI_13376446-S | C20orf98 | −0.02 | −0.07 | −0.17 | −0.02 | −0.08 | 0.00 | −0.14 | −0.07 |
| GI_14042940-S | eIF2A | −0.01 | −0.14 | −0.04 | −0.14 | −0.01 | −0.08 | −0.09 | −0.07 |
| GI_27436965-A | KCNAB1 | −0.10 | −0.05 | −0.09 | −0.07 | −0.09 | −0.03 | −0.08 | −0.07 |
| GI_4757805-S | C16orf7 | −0.06 | −0.10 | −0.15 | −0.05 | −0.02 | −0.02 | −0.10 | −0.07 |
| GI_7662423-S | KIAA0972 | −0.08 | −0.08 | −0.02 | −0.18 | −0.02 | −0.07 | −0.06 | −0.07 |
| GI_34303930-S | MGC20262 | −0.10 | −0.01 | −0.11 | −0.09 | −0.01 | −0.02 | −0.16 | −0.07 |
| GI_4758897-A | PEX16 | −0.07 | −0.11 | −0.20 | −0.04 | −0.02 | 0.00 | −0.06 | −0.07 |
| GI_19923722-S | RPS6KC1 | −0.01 | −0.09 | −0.10 | −0.09 | −0.07 | −0.05 | −0.10 | −0.07 |
| GI_40255224-S | FLJ12892 | −0.05 | −0.01 | −0.02 | −0.15 | −0.10 | −0.04 | −0.10 | −0.07 |
| GI_34222360-S | ATP1A1 | −0.02 | −0.11 | −0.11 | −0.02 | −0.07 | −0.09 | −0.05 | −0.07 |
| GI_5454157-S | VARS2 | −0.04 | −0.14 | −0.14 | −0.04 | −0.02 | −0.09 | −0.02 | −0.07 |
| GI_31657108-S | ZNF282 | −0.04 | −0.13 | −0.04 | −0.03 | −0.02 | −0.18 | −0.04 | −0.07 |
| GI_16933538-A | GLMN | −0.03 | −0.07 | −0.02 | −0.10 | −0.11 | −0.01 | −0.15 | −0.07 |
| GI_41327772-S | DDX46 | −0.13 | −0.07 | −0.09 | −0.05 | −0.05 | −0.03 | −0.06 | −0.07 |
| GI_24431993-S | MGC3234 | −0.06 | −0.07 | −0.06 | −0.07 | −0.03 | −0.05 | −0.13 | −0.07 |
| GI_19718776-S | FEN1 | −0.01 | −0.12 | −0.07 | −0.07 | −0.08 | −0.07 | −0.05 | −0.07 |
| GI_38026914-A | ARHGEF11 | 0.00 | −0.04 | −0.10 | −0.05 | −0.08 | −0.15 | −0.04 | −0.07 |
| GI_38570137-S | MGC15677 | −0.11 | −0.09 | −0.11 | −0.04 | 0.00 | −0.06 | −0.06 | −0.07 |
| GI_34222197-S | C10orf22 | −0.07 | −0.03 | −0.01 | −0.07 | −0.07 | −0.09 | −0.12 | −0.07 |
| GI_12232402-S | FLJ13868 | −0.07 | −0.02 | −0.05 | −0.09 | −0.10 | −0.01 | −0.12 | −0.07 |
| GI_13899304-S | CD99L2 | −0.06 | −0.05 | −0.13 | −0.01 | −0.05 | −0.07 | −0.09 | −0.07 |
| GI_31543933-S | VMP | −0.05 | −0.07 | −0.12 | −0.06 | −0.06 | −0.03 | −0.08 | −0.07 |
| GI_13376430-S | FLJ13397 | −0.02 | −0.04 | −0.04 | −0.09 | −0.10 | −0.03 | −0.13 | −0.07 |
| GI_7669496-S | JWA | −0.04 | −0.04 | −0.07 | −0.04 | −0.11 | −0.02 | −0.14 | −0.07 |
| GI_34147471-S | MGC20781 | −0.01 | −0.10 | −0.11 | −0.12 | −0.05 | −0.02 | −0.06 | −0.06 |
| GI_41146823-S | LOC389197 | −0.10 | −0.06 | −0.07 | −0.06 | −0.08 | −0.02 | −0.06 | −0.06 |
| GI_42734386-S | LOC199692 | −0.03 | 0.00 | −0.04 | −0.04 | −0.12 | −0.03 | −0.20 | −0.06 |
| GI_21362099-S | ELOVL4 | −0.03 | −0.10 | −0.01 | −0.08 | −0.06 | −0.05 | −0.13 | −0.06 |
| GI_18105052-S | RAE1 | −0.05 | −0.08 | −0.07 | −0.02 | −0.08 | −0.11 | −0.03 | −0.06 |
| GI_34222118-S | SYT4 | −0.10 | −0.06 | 0.00 | −0.05 | −0.12 | −0.04 | −0.08 | −0.06 |
| GI_34147419-S | ACBD6 | 0.00 | −0.18 | −0.08 | −0.06 | −0.06 | −0.02 | −0.05 | −0.06 |
| GI_19923214-S | MEF2C | −0.02 | −0.06 | −0.07 | −0.07 | −0.08 | −0.02 | −0.11 | −0.06 |
| GI_4758181-S | DNM1 | −0.08 | −0.09 | −0.12 | −0.01 | −0.05 | 0.00 | −0.08 | −0.06 |
| GI_22748930-S | FBXL14 | −0.05 | −0.02 | −0.03 | −0.07 | −0.05 | −0.02 | −0.20 | −0.06 |
| GI_7706644-S | PME-1 | −0.04 | −0.12 | −0.08 | −0.02 | −0.08 | −0.06 | −0.03 | −0.06 |
| GI_6466453-S | SNCB | −0.10 | −0.09 | −0.07 | −0.01 | −0.11 | −0.05 | −0.02 | −0.06 |
| GI_21361484-S | DKFZP434P1750 | −0.06 | −0.13 | −0.12 | −0.02 | −0.05 | −0.04 | −0.03 | −0.06 |
| GI_7657503-S | RBM9 | −0.02 | −0.11 | −0.03 | −0.07 | −0.04 | −0.07 | −0.09 | −0.06 |
| GI_19718745-A | OSBPL1A | −0.03 | −0.04 | −0.10 | −0.07 | −0.04 | −0.08 | −0.07 | −0.06 |
| GI_18373307-S | RAB40C | 0.00 | −0.06 | −0.11 | −0.04 | −0.02 | −0.10 | −0.10 | −0.06 |
| GI_33598918-A | SCAMP1 | −0.04 | −0.02 | −0.06 | −0.06 | −0.02 | −0.08 | −0.14 | −0.06 |
| GI_21361102-S | SLC25A12 | 0.00 | −0.09 | −0.02 | −0.14 | −0.07 | −0.03 | −0.07 | −0.06 |
| GI_8922070-S | LOC55565 | −0.04 | −0.06 | −0.10 | 0.00 | −0.05 | 0.00 | −0.16 | −0.06 |
| GI_4507212-S | SRP19 | −0.05 | −0.06 | −0.05 | −0.08 | −0.03 | −0.01 | −0.14 | −0.06 |
| GI_40068504-S | BSCL2 | −0.01 | −0.12 | −0.08 | −0.01 | −0.08 | −0.04 | −0.08 | −0.06 |
| GI_13027629-S | DGCR14 | −0.09 | −0.11 | −0.14 | −0.04 | 0.00 | −0.01 | −0.01 | −0.06 |
| GI_9257239-A | SDFR1 | −0.05 | −0.03 | −0.02 | −0.03 | −0.13 | −0.02 | −0.14 | −0.06 |
| GI_25121973-S | LOC151835 | −0.06 | 0.00 | −0.04 | −0.17 | −0.03 | −0.08 | −0.02 | −0.06 |
| GI_5453861-S | PDE4A | −0.03 | −0.09 | −0.09 | −0.12 | −0.03 | −0.02 | −0.02 | −0.06 |
| GI_34222152-S | VSNL1 | −0.14 | −0.03 | −0.02 | −0.02 | −0.09 | −0.02 | −0.08 | −0.06 |
| GI_32490571-S | EPB41L3 | −0.06 | −0.10 | −0.03 | −0.04 | −0.03 | −0.12 | −0.03 | −0.06 |
| GI_13325063-S | CELSR2 | −0.02 | −0.04 | −0.06 | −0.11 | −0.06 | −0.09 | −0.02 | −0.06 |
| GI_27764866-S | SYP | −0.01 | −0.11 | −0.08 | −0.04 | −0.11 | −0.03 | −0.01 | −0.06 |
| GI_37622344-A | ZNF42 | −0.06 | −0.05 | −0.05 | −0.05 | 0.00 | −0.05 | −0.12 | −0.06 |
| GI_10092616-S | PCBP3 | −0.02 | −0.06 | −0.06 | −0.06 | −0.10 | −0.01 | −0.06 | −0.06 |
| GI_31343339-S | FLJ33996 | 0.00 | −0.12 | −0.03 | −0.07 | −0.05 | −0.08 | −0.04 | −0.06 |
| GI_13375816-S | NEIL1 | −0.04 | −0.01 | −0.08 | −0.16 | −0.01 | −0.01 | −0.08 | −0.05 |
| GI_18550284-S | KIAA1912 | −0.03 | −0.01 | −0.03 | −0.05 | −0.11 | −0.03 | −0.13 | −0.05 |
| GI_4507104-S | SNAPC3 | −0.01 | −0.04 | −0.05 | −0.11 | −0.09 | −0.05 | −0.02 | −0.05 |
| GI_20127649-S | KIAA0157 | −0.04 | −0.02 | 0.00 | −0.06 | −0.07 | −0.06 | −0.12 | −0.05 |
| GI_35493938-S | ProSAPiP1 | −0.01 | −0.05 | −0.09 | −0.03 | −0.01 | −0.08 | −0.12 | −0.05 |
| GI_33667083-S | DNAJC9 | −0.01 | −0.02 | −0.04 | −0.10 | −0.12 | −0.05 | −0.05 | −0.05 |
| GI_37555997-S | LOC375663 | −0.09 | −0.01 | −0.07 | −0.08 | −0.03 | 0.00 | −0.09 | −0.05 |
| GI_22748944-S | MGC26690 | −0.04 | −0.07 | −0.08 | −0.04 | 0.00 | −0.01 | −0.13 | −0.05 |
| GI_39725643-A | MR-1 | −0.01 | −0.11 | −0.07 | −0.06 | −0.03 | −0.07 | −0.03 | −0.05 |
| GI_42734431-S | NLK | −0.04 | −0.03 | −0.05 | −0.08 | −0.03 | −0.09 | −0.04 | −0.05 |
| GI_4502286-S | ATP2B1 | −0.09 | −0.02 | −0.08 | −0.05 | −0.01 | −0.04 | −0.07 | −0.05 |
| GI_33391149-S | NPM2 | −0.03 | −0.06 | −0.07 | −0.09 | −0.06 | −0.01 | −0.04 | −0.05 |
| GI_44955932-I | UBQLN1 | −0.06 | 0.00 | 0.00 | −0.08 | −0.06 | −0.10 | −0.07 | −0.05 |
| GI_24308367-S | FLJ38944 | −0.01 | −0.09 | −0.10 | −0.05 | −0.04 | 0.00 | −0.07 | −0.05 |
| GI_37059735-S | CWF19L1 | −0.03 | −0.07 | −0.01 | −0.08 | 0.00 | −0.08 | −0.08 | −0.05 |
| GI_24797146-S | SEPHS2 | −0.05 | −0.07 | −0.05 | −0.04 | −0.06 | −0.04 | −0.05 | −0.05 |
| GI_42476122-S | RUSC1 | −0.01 | −0.04 | −0.03 | −0.04 | −0.08 | −0.04 | −0.10 | −0.05 |
| GI_24307960-S | KIAA0406 | −0.01 | −0.04 | −0.05 | −0.06 | −0.02 | −0.02 | −0.14 | −0.05 |
| GI_40354211-S | PIP3-E | −0.06 | −0.08 | −0.01 | −0.09 | −0.03 | −0.02 | −0.05 | −0.05 |
| GI_34147620-A | AMPD2 | −0.03 | −0.03 | −0.09 | −0.03 | −0.06 | −0.02 | −0.08 | −0.05 |
| GI_21071040-S | CNTNAP2 | −0.02 | −0.06 | −0.04 | −0.07 | −0.05 | −0.05 | −0.04 | −0.05 |
| GI_27436982-S | KCND2 | −0.03 | −0.04 | −0.04 | −0.04 | −0.09 | −0.02 | −0.09 | −0.05 |
| GI_29029532-A | SULT4A1 | −0.01 | −0.13 | −0.03 | −0.05 | −0.06 | −0.06 | 0.00 | −0.05 |
| GI_4507830-S | ULK1 | 0.00 | −0.03 | −0.10 | −0.04 | −0.03 | 0.00 | −0.13 | −0.05 |
| GI_32698821-S | LOC90637 | −0.10 | −0.04 | −0.02 | −0.05 | −0.04 | −0.04 | −0.05 | −0.05 |
| GI_11968046-S | PAF53 | −0.03 | −0.05 | −0.04 | 0.00 | −0.08 | −0.01 | −0.12 | −0.05 |
| GI_45359844-S | G3BP2 | −0.04 | −0.06 | −0.03 | −0.03 | −0.12 | −0.01 | −0.04 | −0.05 |
| GI_34101281-S | SCNN1D | −0.01 | −0.06 | −0.03 | −0.07 | −0.03 | −0.05 | −0.06 | −0.05 |
| GI_34147584-S | DMAP1 | −0.03 | −0.07 | −0.06 | −0.04 | −0.01 | −0.03 | −0.08 | −0.04 |
| GI_37537698-S | LOC147965 | −0.03 | −0.05 | −0.07 | −0.05 | −0.01 | −0.02 | −0.06 | −0.04 |
| GI_24432025-S | FLJ14360 | 0.00 | −0.02 | −0.10 | −0.01 | −0.01 | −0.02 | −0.14 | −0.04 |
| GI_8922197-S | FLJ10038 | 0.00 | −0.13 | −0.03 | −0.06 | −0.01 | −0.02 | −0.05 | −0.04 |
| GI_10938009-A | TSC2 | −0.07 | −0.05 | −0.07 | −0.08 | −0.01 | −0.01 | 0.00 | −0.04 |
| GI_24308110-S | DKFZp564O1863 | −0.05 | −0.03 | −0.04 | −0.07 | −0.07 | −0.01 | −0.02 | −0.04 |
| GI_4826693-S | DGCR2 | −0.04 | −0.06 | −0.10 | 0.00 | −0.03 | −0.02 | −0.02 | −0.04 |
| GI_22035549-S | APBA2 | −0.05 | −0.08 | −0.01 | −0.02 | −0.09 | −0.01 | −0.01 | −0.04 |
| GI_19923443-S | CGI-141 | −0.03 | 0.00 | −0.04 | −0.09 | −0.08 | −0.02 | −0.01 | −0.04 |
| GI_30795206-S | PPP2R5B | −0.05 | −0.05 | −0.08 | −0.05 | −0.04 | 0.00 | 0.00 | −0.04 |
| GI_22027545-S | CACNG3 | −0.05 | −0.01 | −0.03 | −0.02 | −0.07 | −0.04 | −0.04 | −0.04 |
| GI_23065565-S | GPR24 | −0.01 | −0.03 | −0.05 | −0.04 | −0.02 | −0.09 | −0.03 | −0.04 |
| GI_45439315-I | PPIE | −0.02 | −0.09 | −0.03 | −0.01 | −0.04 | −0.05 | −0.01 | −0.04 |
| GI_40255102-S | ZNF488 | −0.05 | −0.09 | −0.03 | −0.01 | 0.00 | −0.05 | −0.01 | −0.03 |
| GI_4505460-S | ENC1 | −0.03 | −0.04 | 0.00 | −0.03 | −0.07 | −0.03 | −0.04 | −0.03 |
| GI_30795230-S | BASP1 | −0.01 | −0.02 | −0.01 | −0.02 | −0.06 | −0.05 | −0.06 | −0.03 |
| GI_4758403-S | FRG1 | −0.03 | −0.03 | −0.02 | −0.02 | −0.04 | −0.03 | −0.03 | −0.03 |
| GI_45439343-I | PPIL5 | −0.02 | −0.02 | −0.04 | −0.06 | 0.00 | −0.02 | −0.03 | −0.03 |
| GI_16445028-S | IGSF8 | −0.05 | 0.00 | −0.07 | −0.01 | 0.00 | −0.02 | −0.04 | −0.03 |
| GI_19913415-A | AP2A1 | 0.00 | −0.02 | −0.03 | −0.02 | −0.02 | −0.06 | −0.04 | −0.03 |
| GI_29126235-S | PGSF1 | −0.02 | −0.01 | −0.03 | −0.01 | 0.00 | −0.05 | −0.01 | −0.02 |

This observation of an overlapping GPI for these 7 PD-associated loci was moreover confirmed in an additional independent dataset of cerebral frontal cortex autopsy brain tissue of 143 individuals (p=1.6 E-3 by resampling statistics: derived from GEO GSE15745).

Function annotation was performed on the gene expression changes that underlie the common GPIs among PD risk variants. Strikingly, among the annotated gene sets most significantly reduced in expression are “mitochondria” functions (FIGS. 8C-8D), consistent with the well-described association of defects in mitochondria with PD pathology (Zheng et al., 2010). Furthermore, the common overlapping transcriptomic signature of gene expression changes associated with these 7 PD risk variants revealed a pattern most similar to the transcriptome changes observed in the context of PD patient brain tissue (relative to unaffected brain tissue; FIG. 8C), rather than to other CNS disorders such as Alzheimer's disease or schizophrenia.

LRRK2 and PARK16 Variants Cooperatively Determine PD Risk

Among the 7 analyzed PD risk locus GPIs, those at the PARK16 and LRRK2 loci were found to be the most similar. Furthermore, variants at these two loci impacted the transcriptome in a non-additive manner, signifying a genetic interaction (as determined by analysis of carriers of both risk variants; FIG. 1D). It was investigated whether these loci similarly genetically interact in terms of their impact on PD risk: namely, whether harboring either a risk (or protective) allele at one of these loci would modify the association of the second locus with PD risk. In an initial study on an Ashkenazi Jewish (AJ) population, the effect of a risk-associated variant at the LRRK2 locus was in fact highly dependent on the presence of the risk variant at the PARK16 locus, and vice versa (FIG. 1E). Such ‘epistasis’ between the LRRK2 and PARK16 loci regarding PD risk was replicated by reanalysis of 3 other independent GWAS, strongly supporting a common mechanism of action (FIG. 1E). Although prior studies have not reported genetic interactions with the common sporadic PD risk-associated variants at the LRRK2 locus, a GWAS of patients who harbor rare familial LRRK2 mutations identified a broad 15 Mb region of Chromosome 1 as harboring a genetic modifier of age of PD onset (Latourelle et al., 2011). It is noted that this region encompassed the PARK16 locus. Meta-analysis in 4 independent sporadic PD GWAS datasets (as above) of the 74 identified SNP variants within this Chromosome 1 region for epistasis with the common LRRK2 SNP variant regarding PD risk identified the PARK16-associated variant as by far the most significantly interacting variant (combined p-value for epistasis: 4.6E-6; FIG. 1F, Table 3).

TABLE 3
LRRK2-PARK16 epistasis meta-analysis.

| | | NGRC | | | NINDS | | |
|---|---|---|---|---|---|---|---|
| SNP | Chr1 pos. | SNP | Int. OR | p | SNP | Int. OR | p |
| rs12063329 | 148269060 | rs32063329 | 0.95 | 0.6179 | rs12063329 | 0.82 | 0.4924 |
| rs6684514 | 154522030 | rs6684514 | 1.04 | 0.6500 | rs6684514 | 0.97 | 0.8855 |
| rs10908495 | 154530564 | rs10908495 | 1.04 | 0.6220 | rs10908495 | 0.96 | 0.8442 |
| rs10908496 | 154530624 | rs10908496 | 1.04 | 0.6428 | rs10908496 | 0.96 | 0.8601 |
| rs10908498 | 154544799 | rs10908498 | 1.04 | 0.6140 | rs10908498 | 0.96 | 0.8442 |
| rs10908502 | 154566706 | rs10908502 | 1.04 | 0.6528 | rs10908502 | 0.96 | 0.8442 |
| rs2789425 | 158216275 | rs2789424 | 1.09 | 0.2855 | rs2789425 | 0.96 | 0.8509 |
| rs2737703 | 158322556 | rs2737703 | 1.02 | 0.8052 | rs2737703 | 1.69 | 0.0218 |
| rs2369406 | 158331042 | rs2369406 | 0.92 | 0.2705 | rs2369406 | 0.72 | 0.1424 |
| rs12057296 | 163609587 | rs12057296 | 1.02 | 0.7676 | rs12057296 | 1.37 | 0.1456 |
| rs3767443 | 165787088 | rs3767443 | 1.14 | 0.0841 | rs3767443 | 0.84 | 0.4414 |
| rs10489248 | 169912212 | rs10489248 | 0.95 | 0.6108 | rs10489248 | 1.22 | 0.5113 |
| rs2014613 | 169914660 | rs10489248 | 0.95 | 0.6108 | rs2014613 | 1.19 | 0.5730 |
| rs3753539 | 169982808 | rs10753181 | 0.93 | 0.3902 | rs3753539 | 1.10 | 0.7107 |
| rs10912977 | 173565736 | rs12565878 | 0.99 | 0.9015 | rs10912977 | 0.76 | 0.2614 |
| rs4652143 | 174250617 | rs4652143 | 1.04 | 0.7926 | rs4652143 | 1.17 | 0.7223 |
| rs11587254 | 174420189 | rs11579161 | 1.11 | 0.5384 | rs4652143 | 1.17 | 0.7223 |
| rs2294254 | 175445331 | rs989536 | 0.99 | 0.9413 | rs2294254 | 1.08 | 0.7621 |
| rs946817 | 176264332 | rs946817 | 1.06 | 0.4805 | rs946817 | 1.19 | 0.4904 |
| rs1281323 | 180368363 | rs1281336 | 1.02 | 0.7906 | rs1281323 | 0.91 | 0.6719 |
| rs2254327 | 180490378 | rs6662373 | 1.00 | 0.9916 | rs2254327 | 1.57 | 0.0487 |
| rs10911659 | 183171884 | rs10911659 | 0.93 | 0.3234 | rs10911659 | 0.91 | 0.6539 |
| rs234092 | 183186797 | rs234092 | 0.92 | 0.3495 | rs234092 | 1.09 | 0.7840 |
| rs234095 | 183197652 | rs234096 | 0.95 | 0.5392 | rs234095 | 1.03 | 0.9132 |
| rs6684195 | 183293436 | rs2378957 | 1.09 | 0.2687 | rs6684195 | 1.01 | 0.9704 |
| rs1200610 | 183418001 | rs1200610 | 0.96 | 0.6659 | rs1200610 | 1.14 | 0.6486 |
| rs1208517 | 183539106 | rs1208517 | 1.00 | 0.9810 | rs1208517 | 1.06 | 0.8244 |
| rs10489485 | 183605173 | rs10489486 | 1.04 | 0.6589 | rs10489486 | 0.88 | 0.6173 |
| rs2186024 | 190696708 | rs2186024 | 1.07 | 0.3788 | rs1286024 | 0.84 | 0.3315 |
| rs2494354 | 192721321 | rs4427392 | 0.96 | 0.6875 | rs7515494 | 1.25 | 0.5291 |
| rs2494312 | 192763213 | rs4427392 | 0.96 | 0.6875 | rs2494312 | 1.21 | 0.5771 |
| rs927724 | 194441413 | rs927724 | 1.08 | 0.5087 | rs2094026 | 2.04 | 0.0601 |
| rs599779 | 197604771 | rs576141 | 0.91 | 0.1984 | rs599779 | 1.12 | 0.5986 |
| rs1898240 | 197613703 | rs1898240 | 0.93 | 0.4634 | rs1898240 | 0.96 | 0.8838 |
| rs487359 | 197648234 | rs571754 | 0.91 | 0.2021 | rs577752 | 0.95 | 0.8005 |
| rs1890133 | 197721501 | rs1890133 | 0.96 | 0.6363 | rs1890133 | 1.42 | 0.1824 |
| rs1400875 | 200088066 | rs2820295 | 0.97 | 0.6978 | rs1400875 | 1.26 | 0.3257 |
| rs2820312 | 200135880 | rs2820312 | 0.97 | 0.7299 | rs2820312 | 1.23 | 0.3759 |
| rs2050935 | 201598170 | rs1977812 | 0.90 | 0.2352 | rs2050935 | 0.78 | 0.3073 |
| rs4950978 | 203291196 | rs1470537 | 1.07 | 0.4049 | rs4950978 | 0.97 | 0.8878 |
| rs1470637 | 203299906 | rs3851287 | 1.08 | 0.4049 | rs1470637 | 1.13 | 0.5737 |
| rs823114 | 203986155 | rs823114 | 0.04 | 0.0137 | rs823114 | 0.01 | 0.0019 |
| rs2802221 | 205643068 | rs2651361 | 1.18 | 0.0289 | rs2802221 | 0.74 | 0.1727 |
| rs643930 | 206224964 | rs560311 | 0.97 | 0.6541 | rs643930 | 0.93 | 0.7161 |
| rs10729481 | 206755035 | rs11119079 | 1.08 | 0.6451 | rs10779481 | 1.15 | 0.8361 |
| rs11119078 | 206758345 | rs11119079 | 1.08 | 0.6451 | rs11119078 | 1.23 | 0.7245 |
| rs1933564 | 207098611 | rs1933564 | 1.06 | 0.4926 | rs1933564 | 1.21 | 0.3966 |
| rs11119439 | 208447880 | rs591594 | 0.97 | 0.7184 | rs11119439 | 0.96 | 0.8673 |
| rs590152 | 208460541 | rs591594 | 0.97 | 0.7184 | rs590152 | 0.96 | 0.8673 |
| rs12124008 | 209710495 | rs12124008 | 1.17 | 0.0333 | rs12124008 | 0.79 | 0.2615 |
| rs3104209 | 211172801 | rs3124669 | 1.18 | 0.0225 | rs104209 | 1.04 | 0.8567 |
| rs487208 | 213209901 | rs1452632 | 0.90 | 0.1258 | rs487208 | 0.76 | 0.1663 |
| rs7548730 | 213911770 | rs7548730 | 1.05 | 0.4447 | rs7548730 | 1.09 | 0.7149 |
| rs7547186 | 215003504 | rs7547186 | 1.05 | 0.4928 | rs7547186 | 1.02 | 0.9243 |
| rs10495064 | 215896812 | rs10495064 | 1.16 | 0.1339 | rs10495064 | 1.63 | 0.1077 |
| rs6673733 | 215982980 | rs6673733 | 0.95 | 0.4659 | rs6673733 | 0.75 | 0.1760 |
| rs2377281 | 216350745 | rs2377781 | 1.07 | 0.3383 | rs2377781 | 1.25 | 0.3612 |
| rs9441867 | 220044136 | rs4433403 | 1.07 | 0.4005 | rs9441867 | 0.74 | 0.2178 |
| rs1341331 | 224702683 | rs12118824 | 0.98 | 0.7591 | rs1341331 | 1.17 | 0.4648 |
| rs750426 | 224715286 | rs4653740 | 1.04 | 0.6729 | rs250426 | 0.78 | 0.3220 |
| rs898833 | 224737622 | rs898833 | 1.03 | 0.7660 | rs898833 | 0.81 | 0.4035 |
| rs11122571 | 228888383 | rs11122571 | 0.92 | 0.2962 | rs11122571 | 1.24 | 0.3892 |
| rs7540252 | 228949072 | rs7540252 | 1.04 | 0.6123 | rs7540252 | 0.76 | 0.2078 |
| rs1316408 | 228952336 | rs1316408 | 1.01 | 0.3658 | rs1316408 | 1.20 | 0.4449 |
| rs7542797 | 229620410 | rs7542797 | 1.19 | 0.0763 | rs7542797 | 0.93 | 0.7858 |
| rs487770 | 232843716 | rs621901 | 0.86 | 0.0944 | rs487770 | 0.95 | 0.8613 |
| rs12746334 | 233775161 | rs12746334 | 1.27 | 0.0768 | rs12746334 | 1.20 | 0.6337 |
| rs12135445 | 237809880 | rs12135445 | 0.99 | 0.9037 | rs12135445 | 0.89 | 0.6888 |
| rs879081 | 238706181 | rs879081 | 1.05 | 0.5440 | rs879081 | 1.35 | 0.2019 |
| rs9287247 | 238713161 | rs9287247 | 0.97 | 0.6916 | rs9287247 | 0.74 | 0.1831 |
| rs2066380 | 238968131 | rs2066380 | 0.86 | 0.0854 | rs2066380 | 0.82 | 0.3853 |
| rs9661248 | 243001296 | rs9661248 | 0.96 | 0.8497 | rs9661248 | 1.33 | 0.3027 |
| rs4654274 | 244735167 | 4654274 | 1.02 | 0.8553 | rs4654274 | 0.98 | 0.9330 |

| AJ | | | MAYO | | | Combined |
|---|---|---|---|---|---|---|
| SNP | Int. OR | p | SNP | Int. OR | p | p |
| rs12063329 | 0.94 | 0.8662 | rs2039800 | 1.02 | 0.9430 | 0.5194 |
| rs6684514 | 0.67 | 0.1444 | rs11264467 | 0.86 | 0.5694 | 0.3901 |
| rs10908495 | 0.67 | 0.1444 | rs11264467 | 0.86 | 0.5694 | 0.3865 |
| rs10908496 | 0.67 | 0.1444 | rs11264467 | 0.86 | 0.5694 | 0.3840 |
| rs10908498 | 0.67 | 0.1444 | rs11264467 | 0.86 | 0.5694 | 0.3896 |
| rs10908502 | 0.67 | 0.1444 | rs11264467 | 0.86 | 0.5694 | 0.3748 |
| rs2789425 | 1.10 | 0.7236 | rs2789425 | 0.78 | 0.4531 | 0.8090 |
| rs2737703 | 1.09 | 0.7647 | rs7512587 | 0.83 | 0.5479 | 0.2630 |
| rs2369406 | 1.27 | 0.3857 | rs2369406 | 1.09 | 0.7563 | 0.4867 |
| rs12057296 | 1.00 | 0.9895 | rs6675585 | 0.75 | 0.3243 | 0.7069 |
| rs3767443 | 1.05 | 0.8429 | rs1229430 | 1.09 | 0.7504 | 0.4612 |
| rs10489248 | 0.92 | 0.7340 | rs10913508 | 1.67 | 0.0889 | 0.4505 |
| rs2014613 | 0.91 | 0.7030 | rs10913508 | 1.67 | 0.0889 | 0.4919 |
| rs3753539 | 0.89 | 0.6261 | rs11808099 | 1.27 | 0.5313 | 0.8613 |
| rs10912977 | 0.75 | 0.3376 | rs6656777 | 0.71 | 0.3228 | 0.1102 |
| rs4652143 | 1.03 | 0.9589 | rs2502841 | 0.81 | 0.6137 | 0.9342 |
| rs11587254 | 1.13 | 0.8516 | rs17351808 | 1.13 | 0.7798 | 0.4763 |
| rs2294254 | 0.91 | 0.7554 | rs12758344 | 1.28 | 0.4058 | 0.7081 |
| rs946817 | 0.96 | 0.9041 | rs7519563 | 0.96 | 0.8925 | 0.5668 |
| rs1281323 | 1.43 | 0.1514 | rs1281338 | 0.68 | 0.1825 | 0.9775 |
| rs2254327 | 1.20 | 0.4666 | rs2254702 | 0.77 | 0.3262 | 0.3934 |
| rs10911659 | 0.67 | 0.1191 | rs234122 | 1.22 | 0.4752 | 0.2542 |
| rs234092 | 0.88 | 0.6704 | rs4650678 | 0.69 | 0.3239 | 0.2998 |
| rs234095 | 0.74 | 0.3455 | rs234122 | 1.22 | 0.4752 | 0.7135 |
| rs6684195 | 1.07 | 0.7890 | rs12030554 | 0.95 | 0.8735 | 0.5315 |
| rs1200610 | 1.33 | 0.3384 | rs7413268 | NA | NA | 0.5710 |
| rs1208517 | 1.57 | 0.1727 | rs6424975 | 1.16 | 0.5814 | 0.2907 |
| rs10489486 | 1.17 | 0.5726 | rs6689206 | 1.20 | 0.4825 | 0.5581 |
| rs2186024 | 0.91 | 0.7059 | rs12119534 | 0.87 | 0.6666 | 0.6530 |
| rs2494354 | 1.38 | 0.6329 | rs2494155 | NA | NA | 0.6841 |
| rs2494312 | 1.53 | 0.5164 | rs2494315 | NA | NA | 0.6424 |
| rs927724 | 1.38 | 0.3749 | rs7518775 | 0.66 | 0.3943 | 0.1977 |
| rs599779 | 0.88 | 0.6135 | rs556744 | 1.13 | 0.7071 | 0.6567 |
| rs1898240 | 0.94 | 0.8383 | rs16844836 | 1.10 | 0.7876 | 0.6840 |
| rs487359 | 0.65 | 0.0974 | rs590448 | 0.88 | 0.6975 | 0.0696 |
| rs1890133 | 1.45 | 0.3036 | rs7538527 | 0.95 | 0.8832 | 0.3837 |
| rs1400875 | 0.93 | 0.7980 | rs2644112 | 1.40 | 0.2747 | 0.4743 |
| rs2820312 | 0.97 | 0.9146 | rs2820312 | 1.13 | 0.7156 | 0.6901 |
| rs2050935 | 0.83 | 0.5526 | rs12406229 | 0.71 | 0.3761 | 0.0653 |
| rs4950978 | 0.77 | 0.3270 | rs3862948 | 0.71 | 0.3380 | 0.5629 |
| rs1470637 | 0.75 | 0.2661 | rs9787334 | 0.71 | 0.2640 | 0.6768 |
| rs423114 | 0.14 | 0.0414 | rs911154 | 0.41 | 0.0779 | 4.64E−06 |
| rs280221 | 0.63 | 0.0947 | rs966256 | 1.53 | 0.1499 | 0.7680 |
| rs643930 | 1.52 | 0.1195 | rs658347 | 1.07 | 0.7857 | 0.6112 |
| rs10779481 | 0.85 | 0.7714 | rs11119076 | NA | NA | 0.8277 |
| rs11119078 | 1.24 | 0.7355 | rs11119079 | NA | NA | 0.5064 |
| rs1933564 | 0.83 | 0.4452 | rs12043779 | 1.05 | 0.8821 | 0.6460 |
| rs11119439 | 1.15 | 0.5851 | rs11119426 | 1.08 | 0.8554 | 0.9201 |
| rs590152 | 1.15 | 0.5851 | rs845451 | 1.29 | 0.3782 | 0.6529 |
| rs12124008 | 1.46 | 0.1492 | rs11580728 | 0.88 | 0.7044 | 0.3009 |
| rs3104209 | 1.77 | 0.0290 | rs3104212 | 0.69 | 0.2494 | 0.0807 |
| rs487208 | 1.24 | 0.4067 | rs1890007 | 0.77 | 0.4210 | 0.1485 |
| rs7548730 | 1.64 | 0.0655 | rs7518358 | 1.09 | 0.7963 | 0.1064 |
| rs7547186 | 1.94 | 0.0244 | rs11572775 | 1.47 | 0.2693 | 0.0387 |
| rs10495064 | 0.79 | 0.6309 | rs10495065 | 1.10 | 0.7549 | 0.1417 |
| rs6673733 | 0.62 | 0.0705 | rs17046838 | 1.41 | 0.2314 | 0.1779 |
| rs2377781 | 1.71 | 0.0428 | rs10863375 | 1.29 | 0.3272 | 0.0148 |
| rs9441857 | 0.73 | 0.2379 | rs4846353 | 0.96 | 0.3905 | 0.3927 |
| rs1341331 | 0.60 | 0.0442 | rs16845973 | 0.76 | 0.3276 | 0.1993 |
| rs750426 | 0.67 | 0.2191 | rs750426 | 1.01 | 0.9805 | 0.3754 |
| rs898833 | 0.67 | 0.2344 | rs250426 | 1.01 | 0.9805 | 0.3946 |
| rs11122571 | 1.37 | 0.2126 | rs2296800 | 0.98 | 0.9581 | 0.6134 |
| rs7540252 | 0.88 | 0.6022 | rs2282319 | 0.91 | 0.7398 | 0.4219 |
| rs1316408 | 1.30 | 0.3477 | rs12082061 | 1.03 | 0.9301 | 0.3272 |
| rs7542797 | 0.77 | 0.4822 | rs10864669 | 0.93 | 0.7780 | 0.7962 |
| rs487770 | 0.64 | 0.3261 | rs607368 | 1.40 | 0.3102 | 0.3642 |
| rs12746334 | 0.61 | 0.2846 | rs11577962 | NA | NA | 0.4971 |
| rs12135445 | 0.84 | 0.6462 | rs16838380 | 0.85 | 0.6353 | 0.4670 |
| rs879081 | 1.12 | 0.6495 | rs4659570 | 1.02 | 0.9501 | 0.2301 |
| rs9287247 | 0.69 | 0.1627 | rs882869 | 0.99 | 0.9792 | 0.1152 |
| rs2066380 | 0.80 | 0.3605 | rs11802581 | 1.22 | 0.5513 | 0.1461 |
| rs9661248 | 1.10 | 0.7731 | rs4658608 | 1.49 | 0.3460 | 0.3002 |
| rs4654274 | 1.02 | 0.9341 | rs2184975 | 0.84 | 0.5095 | 0.8109 |

Taken together, these data strongly support a genetic interaction between LRRK2 and PARK16 that initially impacts human CNS tissue physiology, as reflected by the transcriptome signature in unaffected carriers, and ultimately favors PD pathology in a small subset of individuals at risk.

Evidence of a LRRK2-RAB7L) Pathway

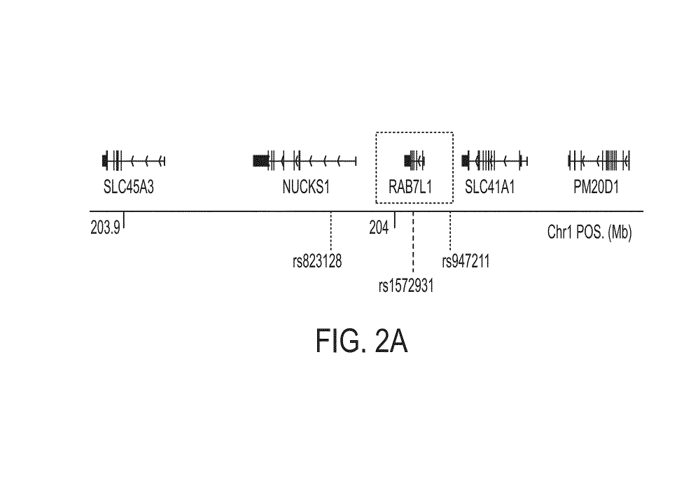

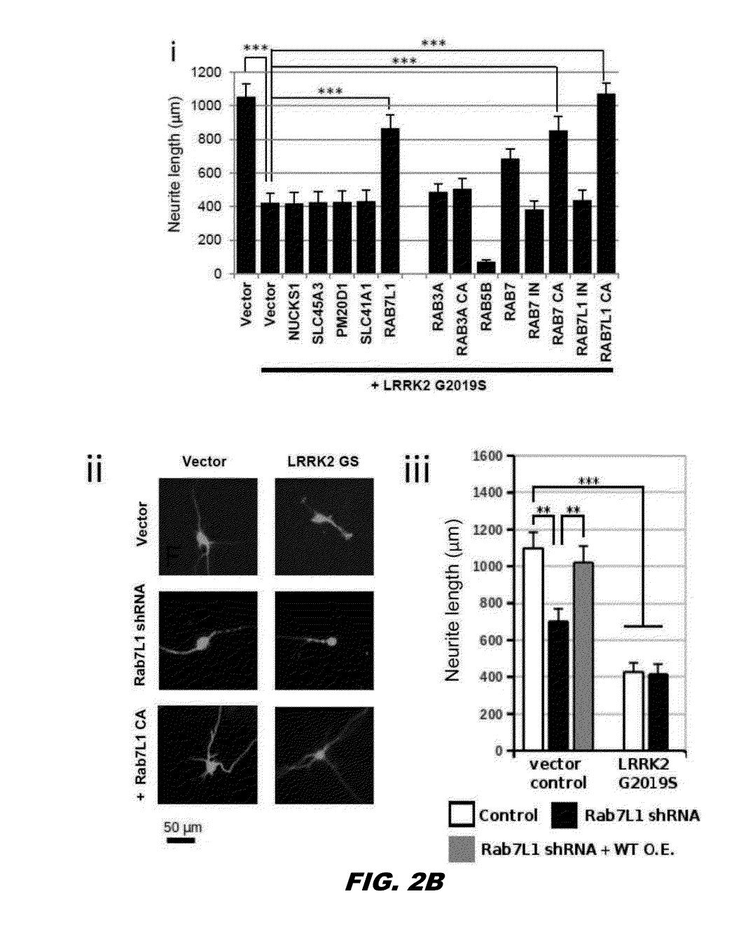

As 5 candidate genes are present within the PARK16 locus (SLC45A3, NUCKS, RAB7L1, SLC41A1, and PM20D1), each of the genes were experimentally screened for a functional interaction with LRRK2 (FIG. 2A). A previously-described primary rat neuron in vitro culture model was used, in which transient expression of familial PD-associated LRRK2 G2019S or R1441C mutant alleles leads to a marked reduction in neurite process length (MacLeod et al., 2006). Overexpression of RAB7L1, but not other genes in the PARK16 locus, significantly suppressed the LRRK2 mutation-induced neurite length phenotype (FIG. 2B). RAB7L1 did not modify neurite length in the context of overexpression of wild-type LRRK2 (FIG. 2A). RAB7L1 is a small cytosolic GTPase, structurally distinct from RAB7 despite its name (also known as RAB29) (Shimizu et al., 1997). One of ˜60 small GTPases in the human genome, RAB7L1 has previously been shown to localize primarily to the Golgi apparatus and implicated in vesicular sorting in the context of Salmonella or Hepatitis C infection (Berger et al., 2009; Spano et al., 2011). But the function of RAB7L1 in CNS neurons remains unknown. Orthologues of RAB7L1 in other organisms, including C. elegans Glo-1 and Drosophila melanogaster Lighloid, have been implicated in trafficking to lysosome-related organelles (Hermann et al., 2005) and in the regulation of neurite process length (Grill et al., 2007), reminiscent of LRRK2 mutant phenotypes (MacLeod et al., 2006). Thus this gene was of particular interest.

Because GTPases such as RAB7L1 are typically only active in the GTP-bound state, mutant forms were generated that are constitutively active (CA; Q67L; this mutation is deficient in GTPase activity) or inactive (IN; T2 IN; a mutation within the presumptive GTP binding site). As expected, overexpression of the CA RAB7L1, but not IN RAB7L1, significantly suppressed the LRRK2 mutation-induced neurite length phenotype. Of other Rab family members, expression of RAB3A or RAB5A failed to rescue the phenotype, whereas RAB7 CA was effective in suppressing the process length shortening induced by LRRK2 mutation (FIG. 2B). In contrast to RAB7L1 overexpression, knockdown of RAB7L1 alone led to a significant reduction in neurite process length, similar to the effect of the LRRK2 G2019S mutant expression (FIG. 2B, 9B).

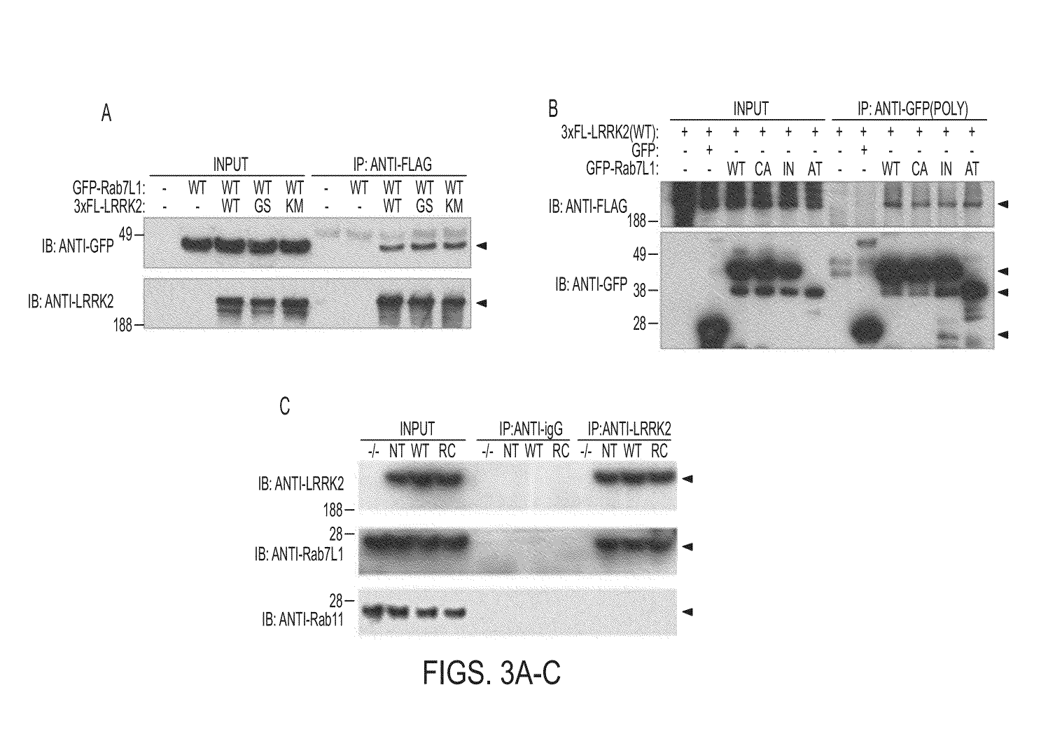

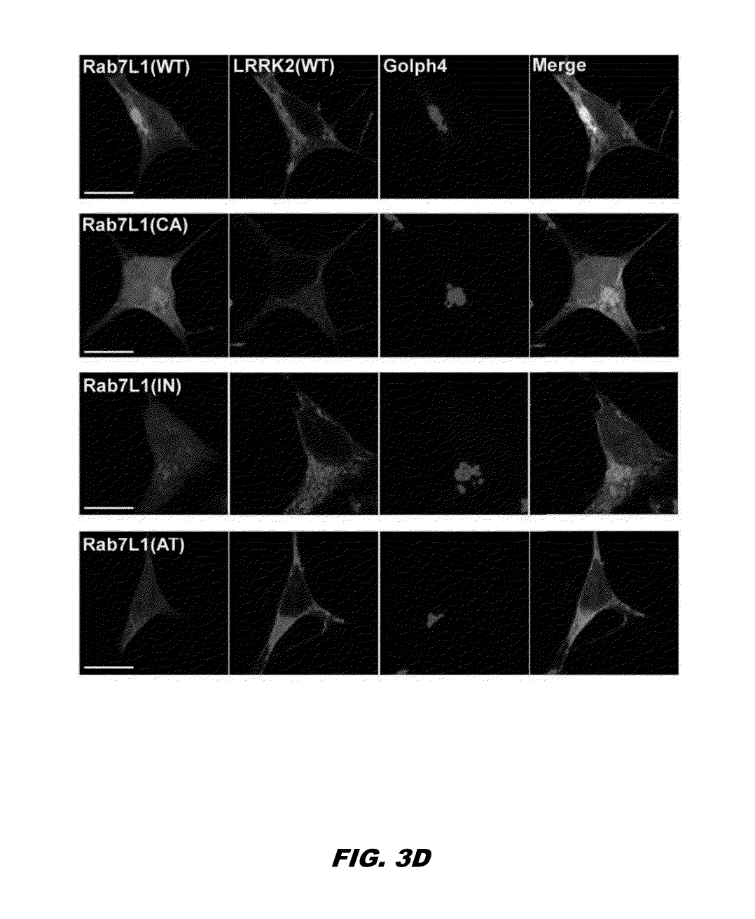

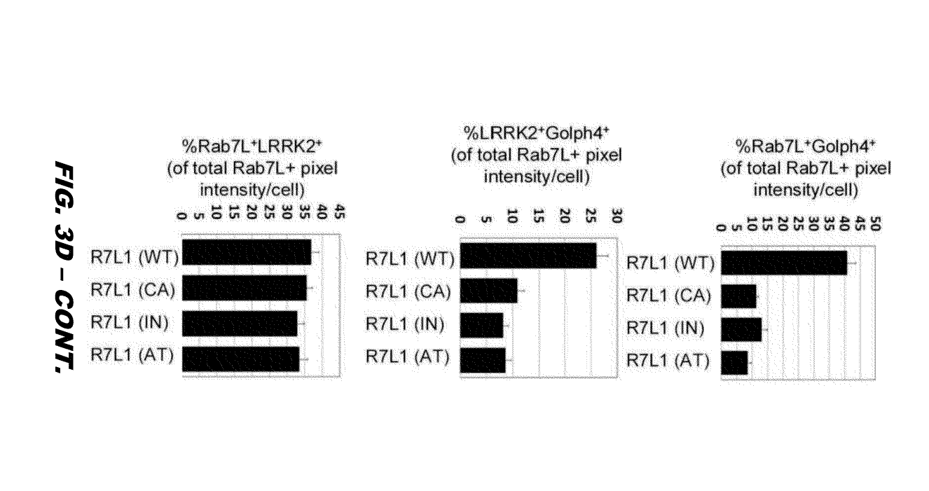

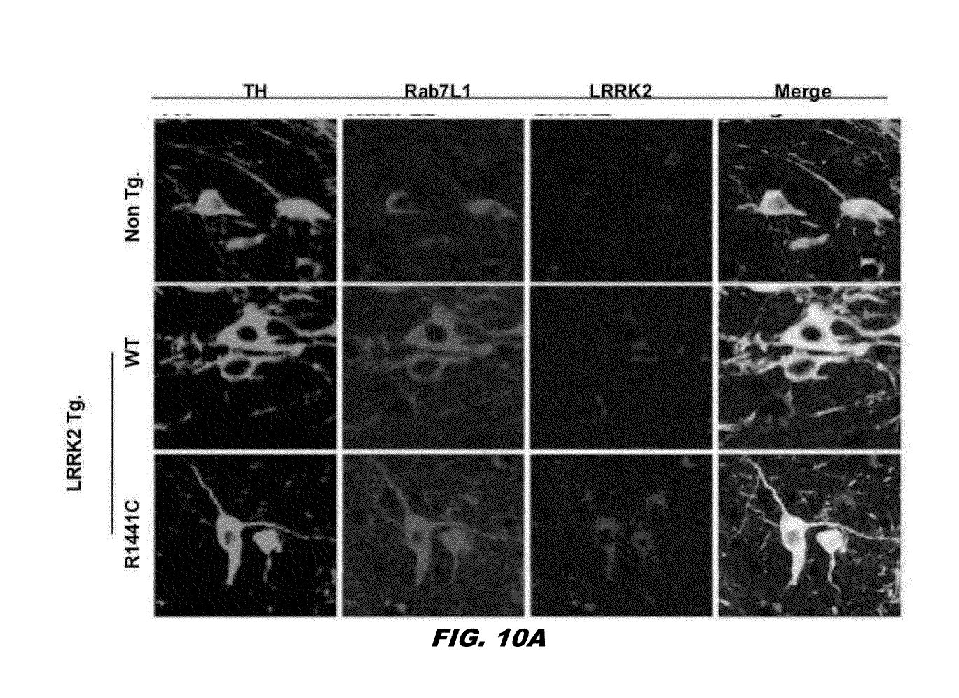

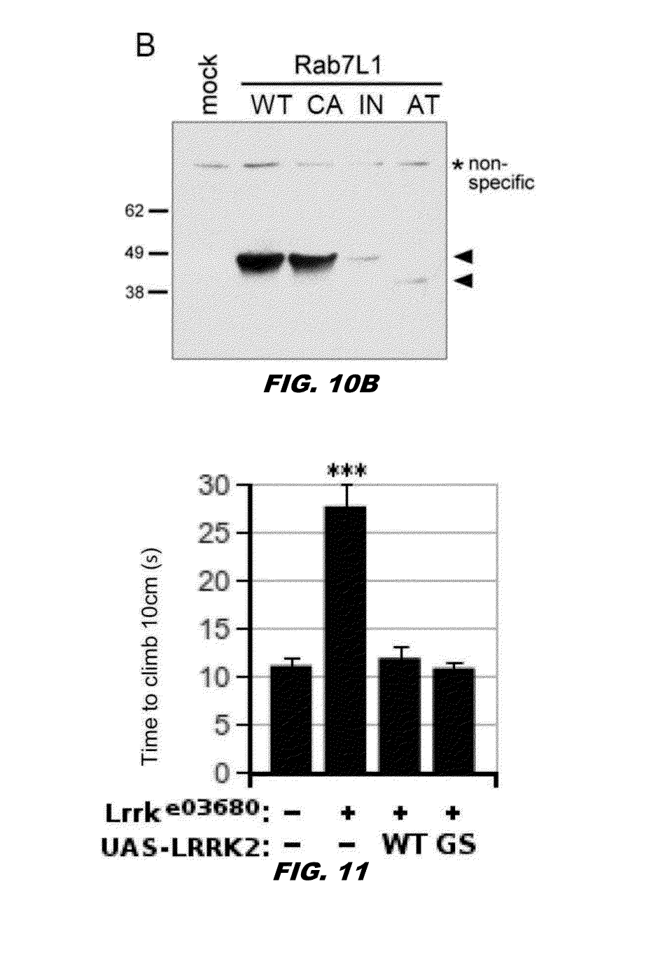

Next more direct evidence of a physical interaction between LRRK2 and RAB7L1 was sought and thus co-immunoprecipitation studies were performed. Epitope-tagged forms of LRRK2 and RAB7L1 (3×Flag-LRRK2 and GFP-RAB7L1) were co-transfected into HEK293T cells, and after 48 hrs, cell lysates were immunoprecipitated with an anti-Flag antibody and then probed for RAB7L1. Flag-immunoprecipitation of LRRK2 effectively co-precipitated RAB7L1 (FIG. 3A). The interaction did not appear to be altered by the presence of the G2019S mutant, or using a kinase-dead variant K1906M of LRRK2 (MacLeod et al., 2006). Similarly, immunoprecipitation of RAB7L1 with an antibody to the GFP tag co-precipitated LRRK2 only in the presence of RAB7L1-GFP (FIG. 3B). To probe for an interaction between LRRK2 and RAB7L1 in a more physiological context, RAB7L1 protein was examined in brain lysates from transgenic mice that harbor human wild-type LRRK2 or a familial PD mutant form of LRRK2, R1441C, within a large bacterial artificial chromosome (BAC) construct. Transgenic LRRK2 is broadly expressed throughout the CNS of these mice, although at relatively low levels (FIG. 10A). Brain tissue lysates were immunoprecipitated for LRRK2 protein with a rabbit monoclonal antibody. Western blotting of the lysates for RAB7L1 demonstrated co-immunoprecipitation of RAB7L1 (FIG. 3C).

In vitro fluorescence microscopy studies were consistent with the presence of RAB7L1 and LRRK2 in common subcellular compartments. GFP-tagged RAB7L1, transfected into SH-SY5Y cells, localized primarily to the Golgi apparatus (as identified with the Golph4 marker), as well as along tubular structures emerging from Golgi apparatus, consistent with prior reports (Spano et al., 2011). LRRK2 staining appeared more diffuse than RAB7L1 but there was significant overlap (FIG. 3D). In contrast to the wild-type form, the RAB7L1 CA or IN mutant forms appeared more diffusely localized through the cytoplasm, as did a RAB7L1 alternative transcript (AT) deficient in the predicted GTP-binding region (FIG. 3D); accumulation of the IN and AT mutant proteins was significantly reduced (FIGS. 3D and 10B).

In Vivo Analysis of a LRRK2-RAB7L1 Pathway in Drosophila Dopamine Neurons

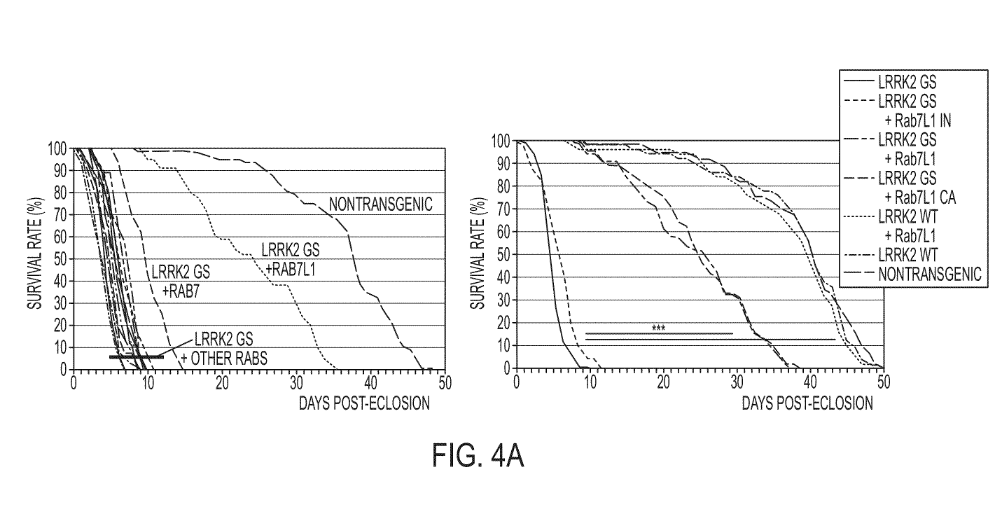

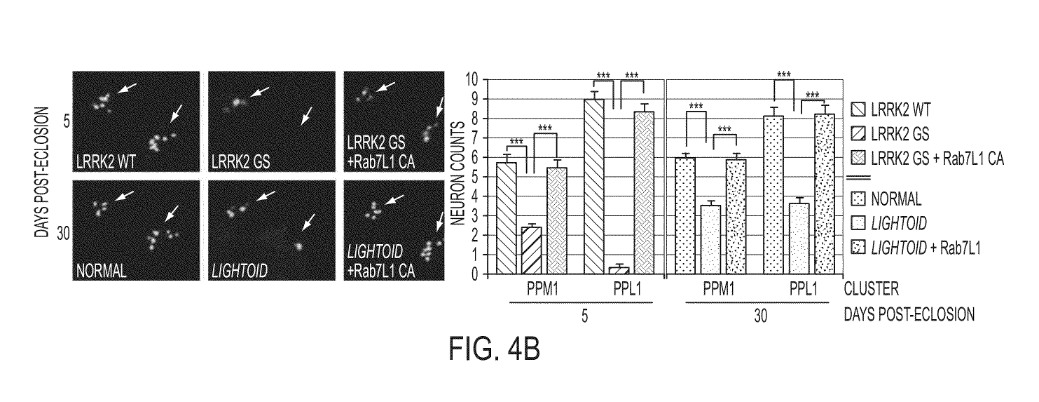

To pursue potential mechanisms of LRRK2 pathology in vivo, a Drosophila model was established. Although transgenic mouse models expressing mutant LRRK2 have been widely described (Andres-Mateos et al., 2009; Li et al., 2009; Piccoli et al., 2011; Tong et al., 2009), these do not show consistent neurodegenerative phenotypes. Dopamine neuron-selective expression of human familial PD-associated G2019S-mutant LRRK2—using either a tyrosine hydroxylase (TH) (Friggi-Grelin et al., 2003) or dopa decarboxylase (DDC) promoter-Gal4 driver (Fischer et al., 1988)—induced premature mortality of young adult animals (FIG. 4A; nontransgenic mean lifespan 37.1 days+/−1; G2019S mean lifespan 4.8 days+/−0.2), akin to previous reports (Ng et al., 2009). In contrast, transgenic expression of wild-type human LRRK2 did not lead to a discernible phenotype. Furthermore, expression of the mutant G2019S LRRK2 transgene in several other cell types, including motor neurons, eye tissues, or muscles (using a variety of promoter-Gal4 driver constructs), failed to lead to a discernible effect on survival or otherwise.

Subsequently a targeted screen for potential genetic modifiers of the LRRK2 G2019S mutant phenotype was performed, based on the idea that LRRK2 may modify a specific intracellular trafficking process, and focused on RAB7L1. A series of 16 Drosophila Rab genes, (see Table 4; out of 33 identified in Drosophila), or CA or IN forms of these (Zhang et al., 2007), were investigated.

TABLE 4
Rab GTPase genes screen for a rescue of the LRRK2 G2019S
phenotype in Drosophila.

| | | Average | | |
|---|---|---|---|---|
| | | adult | | |
| TH-driven | Transgene | lifespan | | |
| Rab GTPase | mutation | (days) | SEM | n |
| Rab1CA | Q70 | 5.9 | 0.43 | 23 |
| Rab2CA | Q65 | 4.7 | 0.44 | 27 |
| Rab3CA | Q80 | 7 | 0.5 | 22 |
| Rab4CA | Q67 | 6 | 0.36 | 21 |
| Rab5CA | Q88 | 5.9 | 0.37 | 20 |
| Rab6CA | Q71 | 5.6 | 0.44 | 22 |
| Rab7WT | n/a | 10.2 | 0.59 | 21 |
| Rab7L1 DN | T33 | 5.6 | 0.53 | 22 |
| Rab7L1WT | n/a | 23.3 | 1.09 | 52 |
| Rab7L1CA | Q79 | 24 | 1.11 | 45 |
| Rab8CA | Q67 | 6.8 | 0.39 | 21 |
| Rab9CA | Q71 | 5.3 | 0.4 | 23 |
| Rab10WT | n/a | 5.6 | 0.39 | 22 |
| Rab14CA | Q94 | 6.6 | 0.37 | 20 |
| Rab18CA | A64 | 4.6 | 0.42 | 20 |
| Rab23CA | Q96 | 6.7 | 0.49 | 20 |
| RabX2CA | D66 | 4.8 | 0.4 | 20 |
| RabX4CA | Q67 | 5.9 | 0.52 | 22 |

Briefly, LRRK2 G2019S mutants were mated with a panel of previously described transgenic Drosophila strains that allow for overexpression of wild-type (WT) or constitutively active (CA), forms of the Rab genes (Zhang et al., 2007), using a standard balancer chromosome-based mating scheme. Co-expression of a majority of these Rab transgenes with LRRK2 within dopamine neurons produced no effect on the survival of animals co-expressing LRRK2 G2019S (FIG. 4A; Table 4). In contrast, overexpression of wild-type and CA forms of the Drosophila RAB7L1 orthologue (termed lightoid) afforded a dramatic rescue of the LRRK2 G2019S premature mortality phenotype (mean lifespan 24.0 days+/−1 for the CA; FIG. 4A). Of note, among the other Rabs screened, only Rab7 led to a statistically significant—albeit much weaker—survival benefit (mean lifespan 14.3 days+/−0.6). Rab1, which was previously found to rescue a defect in vesicular trafficking to the Golgi apparatus in alpha-Synuclein overexpression models of PD (Cooper et al., 2006), did not rescue the LRRK2 defect, suggesting distinct mechanisms.

Next, dopamine neuron survival at the dorsomedial posterior protocerebral (PPM1) and dorsolateral posterior protocerebral (PPL1) clusters of Drosophila CNS mushroom bodies was quantified in terms of the loss of expression of a dopamine neuron-specific nuclear localization signal (NLS)-GFP marker protein, using fluorescent confocal microscopy analysis of whole mounted tissue. Expression of LRRK2 G2019S, but not the WT form, led to the preferential loss of neurons in the dorsomedial cluster, reminiscent of the phenotype in other Drosophila models of PD (Feany and Bender. 2000). Co-expression of CA RAB7L1 rescued the LRRK2 G2019S dopamine neuron loss phenotype (FIG. 4B). Deficiency of the RAB7L1 orthologue (in lightoid homozygous mutants) selectively in dopamine neurons by expression of an siRNA construct (Dietzl et al., 2007), led to a significant loss of dopamine neurons (FIG. 4B).

PARK16 Risk Variants Modify RAB7L1 Splicing and Expression

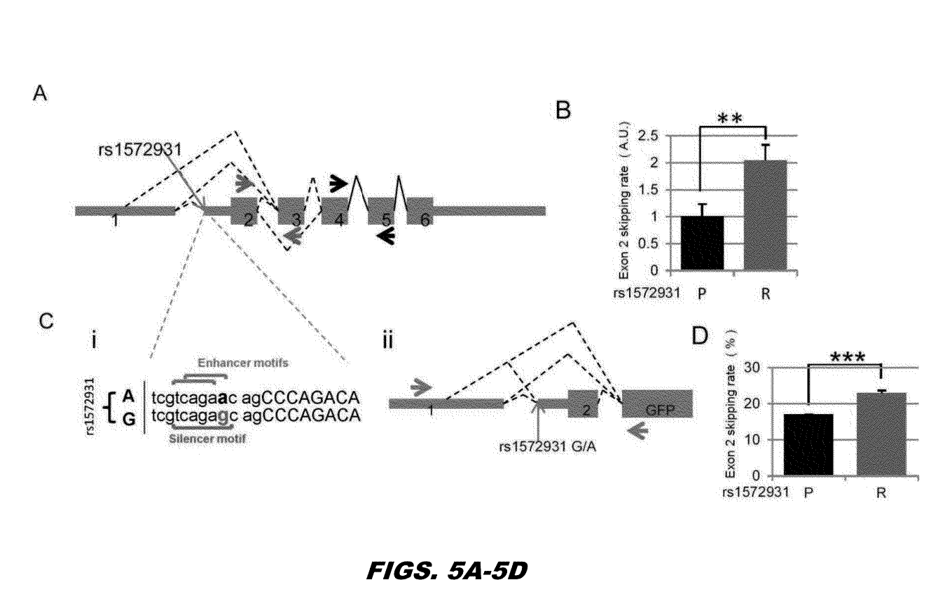

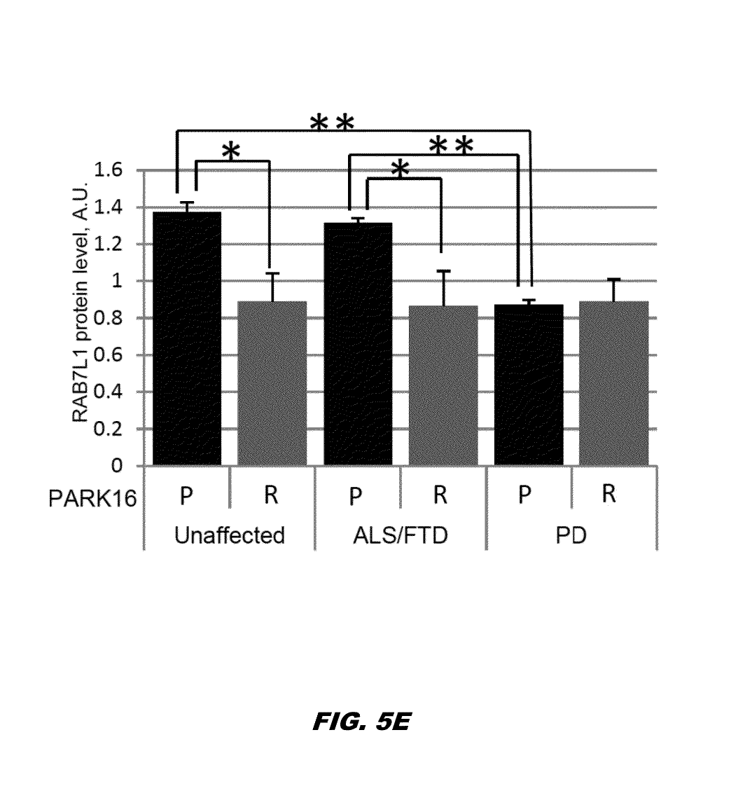

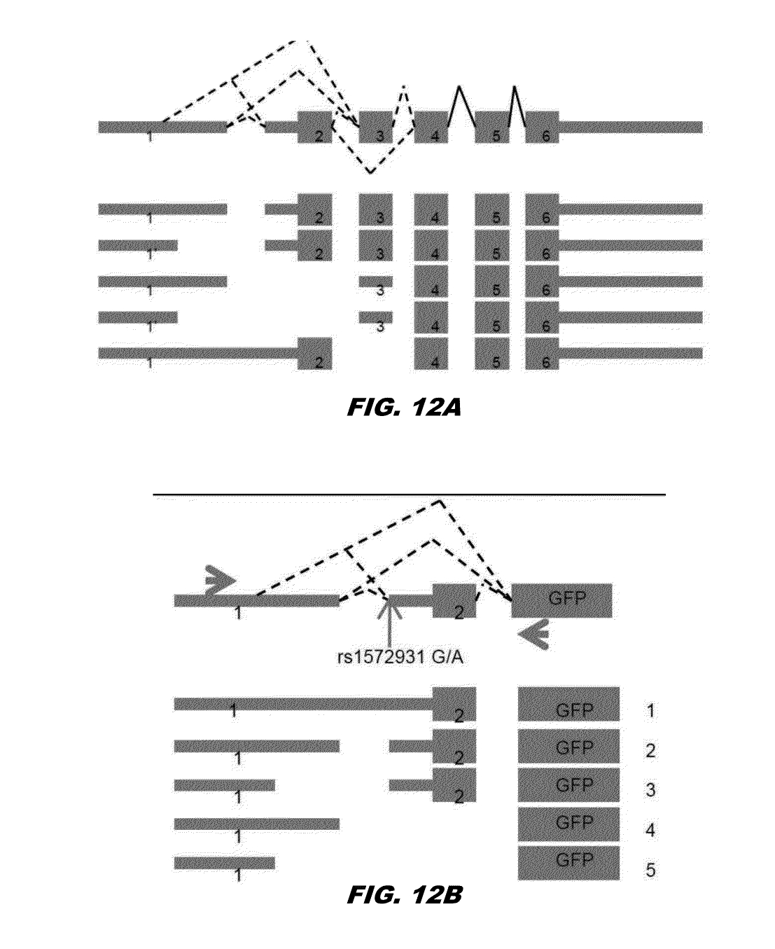

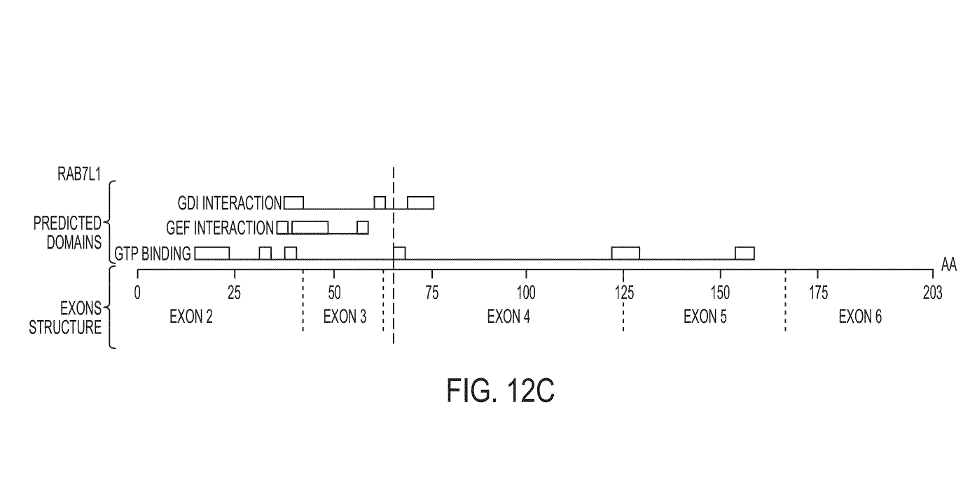

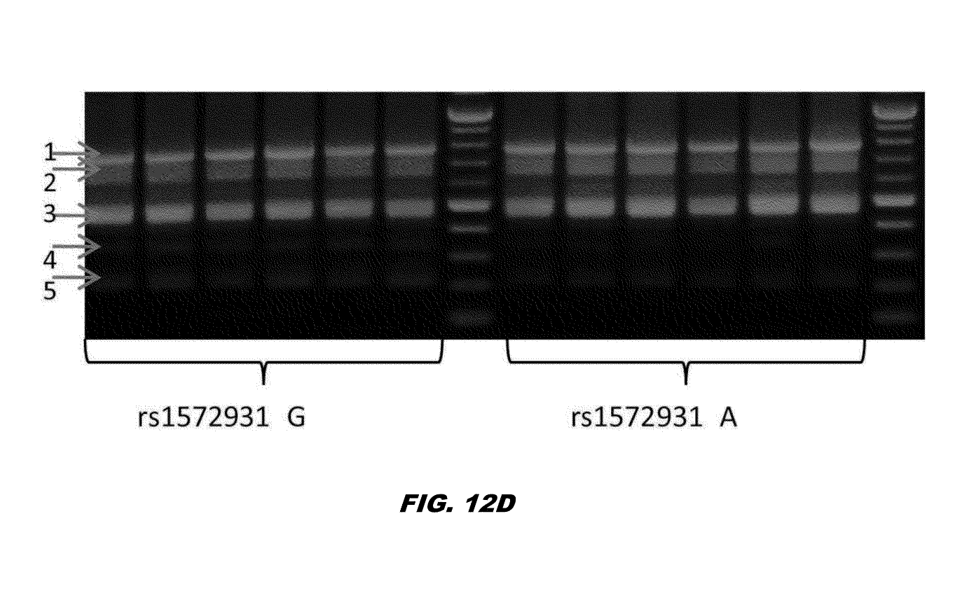

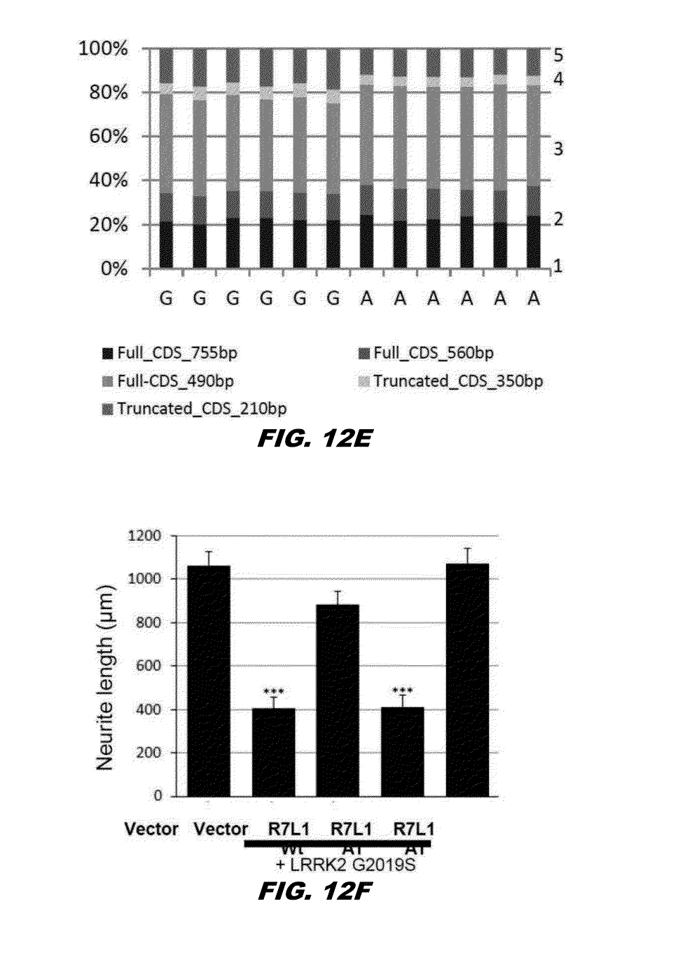

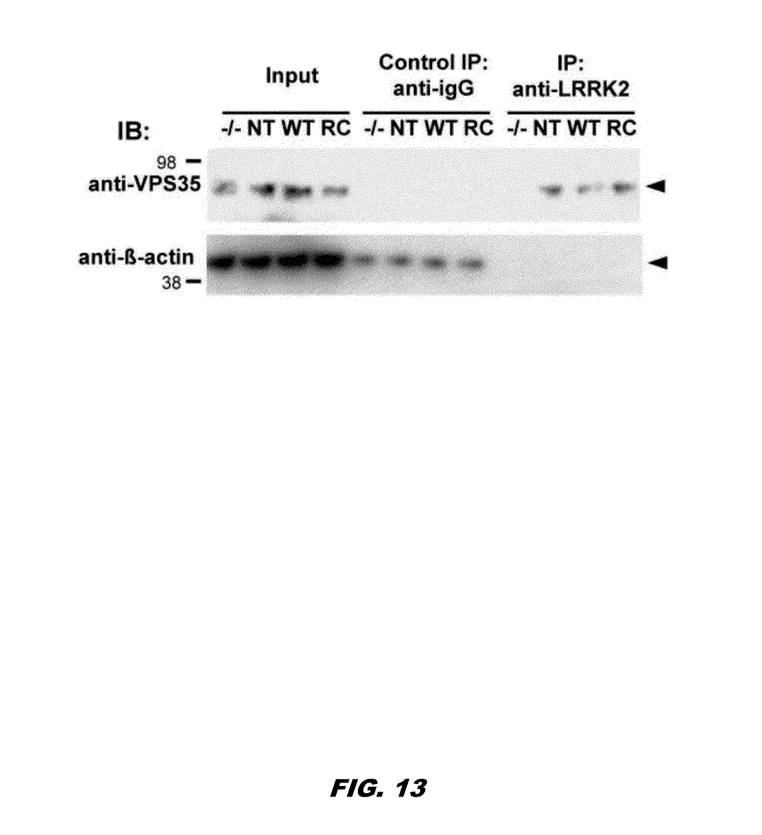

The combination of human brain transcriptomic, human genetic, and model system studies support a role for PARK16, and specifically the PARK16 locus gene RAB7L1, in a pathway with LRRK2. Next possible molecular mechanisms at play at the PARK16 locus that may be responsible for a link between common genetic variants, RAB7L1 function, and PD risk were investigated. A challenge to this is that typically many variants at a given chromosomal location are so closely associated (in ‘linkage dysequilibrium’) so as to make impossible the identification of which is truly ‘causal’ rather than just coincidental. On reanalysis of existing genome-wide splicing data from human lymphoblasts (Montgomery et al., 2010), the PD-associated PARK16 haplotype was found to be associated with alternative splicing of RAB7L1, characterized by the skipping of exons 2 and 3. It is noted that a common SNP variant within the PARK16 locus, rs1572931, that is in linkage dysequilibrium with SNP rs947211 (Hamza et al., 2010) and thus similarly linked to PD risk, falls precisely within regulatory sequences for splicing at the Intron1-exon2 boundary (FIG. 5A). Akin to the lymphoblast transcriptome splicing data, analysis of a set of human cortical brain samples revealed that the rs1572931 genotype is similarly associated with modified splicing of RAB7L in human forebrain (FIGS. 5B, 12A; see Table 6), where the protective PARK16 haplotype is associated with increased exon 2 inclusion in RAB7L1 mRNA. Based strictly on human gene expression data, a causal role for SNP rs1572931 in altered splicing of RAB7L1 cannot be directly assigned (as other SNPs in linkage disequilibrium could be responsible for the observed association). Thus the causal effect of rs1572931 was evaluated using minigene reporter vectors that harbor either the risk-associated or protective allele at rs1572931, but are otherwise identical (FIGS. 5Cii, 12B). Upon transfection into SH-SY5Y human neuroblastoma cells, the rs1572931 risk allele led to increased RAB7L1 exon 2 skipping relative to the protective allele (FIGS. 5D, 12C-E).

TABLE 6
Human Brain Cortical Samples.

| Brain ID | Age | Sex | Status | rs1572931 |
|---|---|---|---|---|
| B1 | 80 | F | Unaff. | AA |
| B2 | 82 | M | LBD | AA |
| B3 | 56 | F | ALS | GA |
| B4 | 62 | F | ALS | GA |
| B5 | 67 | M | ALS | GA |
| B6 | 72 | M | Unaff. | GA |
| B7 | 87 | M | Unaff. | GA |
| B8 | 57 | F | Unaff. | GA |
| B9 | 84 | M | Unaff. | GA |
| B10 | 58 | M | FTD | GA |
| | | | MND | |
| B11 | 85 | M | FTD | GA |
| | | | MND | |
| B12 | 87 | F | PD | GA |
| B13 | 84 | M | PD/D | GA |
| B14 | 80 | M | PD/D | GA |
| B15 | 68 | F | PSP | GA |
| B16 | 83 | M | AD | GG |
| B17 | 89 | F | AD | GG |
| B18 | 79 | F | AD | GG |
| B19 | 89 | F | AD | GG |
| B20 | 89 | F | AD | GG |
| B21 | 83 | M | AD | GG |
| B22 | 73 | M | AD | GG |
| B23 | 86 | F | AD | GG |
| B24 | 75 | F | AD | GG |
| B25 | 62 | F | AD | GG |
| B26 | 89 | F | AD | GG |
| B27 | 89 | F | AD | GG |
| B28 | 89 | M | AD | GG |
| B29 | 88 | M | AD | GG |
| B30 | 76 | M | AD | GG |
| B31 | 80 | M | ALS | GG |
| B32 | 64 | M | ALS | GG |
| B33 | 60 | M | ALS | GG |
| B34 | 79 | F | ALS | GG |
| B35 | 66 | M | ALS | GG |
| B36 | 88 | F | ALS | GG |
| B37 | 71 | F | ALS | GG |
| B38 | 76 | F | Unaff. | GG |
| B39 | 57 | M | Unaff. | GG |
| B40 | 80 | M | Unaff. | GG |
| B41 | 65 | F | Unaff. | GG |
| B42 | 62 | M | Unaff. | GG |
| B43 | 89 | M | Unaff. | GG |
| B44 | 89 | M | Unaff. | GG |
| B45 | 57 | F | Unaff. | GG |
| B46 | 52 | F | Unaff. | GG |
| B47 | 80 | M | FTD | GG |
| B48 | 61 | M | FTD | GG |
| B49 | 74 | M | FTD | GG |
| B50 | 50 | M | FTD | GG |
| | | | MND | |
| B51 | 77 | M | FTD | GG |
| B52 | 73 | M | FTD | GG |
| B53 | 84 | M | LBD | GG |
| B54 | 80 | M | LBD | GG |
| B55 | 76 | F | PD | GG |
| B56 | 83 | M | PD | GG |
| B57 | 74 | M | PD | GG |
| B58 | 80 | F | PD | GG |
| B59 | 77 | F | PD | GG |
| B60 | 80 | F | PD | GG |
| B61 | 85 | M | PD | GG |
| B62 | 77 | M | PD | GG |
| B63 | 77 | F | PD/D | GG |
| B64 | 84 | F | PD/D | GG |
| B65 | 81 | F | PD/D | GG |
| B66 | 69 | F | PD/D | GG |
| B67 | 65 | M | PD/D | GG |
| B68 | 72 | F | PD | GG |

Exon skipping is predicted to lead to the formation of a truncated form of RAB7L1 protein that lacks the predicted GTP-binding domain in the amino-terminal region (FIG. 12C). Overexpression of this truncated form leads to low level accumulation of a shortened protein product (FIG. 10B), and reduced localization to the Golgi apparatus (FIG. 3D); although the shortened product can bind with LRRK2 protein (FIG. 3B), expression of this truncation mutant in primary neurons failed to rescue the reduced neurite length phenotype associated with G2019S mutant LRRK2 (FIG. 12F), whereas expression of the wild-typeRAB7L1 effectively rescued the phenotype. Consistent with these in vitro findings, a significant reduction in full-length RAB7L1 protein was observed in cerebral cortex tissue from unaffected individuals who carry the PARK16 risk allele, when compared to non-carrier individuals (FIG. 5E). It is noted that a similar reduction is seen in PD patient cerebral cortex tissue regardless of the PARK16 genotype. This appears specific to PD, as no such decrease is observed in tissue from patients suffering from other neurodegenerative disorders examined (frontotemporal dementia or amyotrophic lateral sclerosis) who do not carry the PARK16 risk allele (FIG. 5E). Taken together, these findings argue in favor of a post-transcriptional (splicing) mechanism of action for the PARK16 PD risk variant's impact on RAB7L1 levels. However, given the linkage disequilibrium structure of the region, additional transcriptional regulatory effects may exist (Gan-Or et al., 2012).

Lysosomal Changes and Retromer-Associated Sorting Defects in LRRK2 and RAB7L1 Mutant Neurons

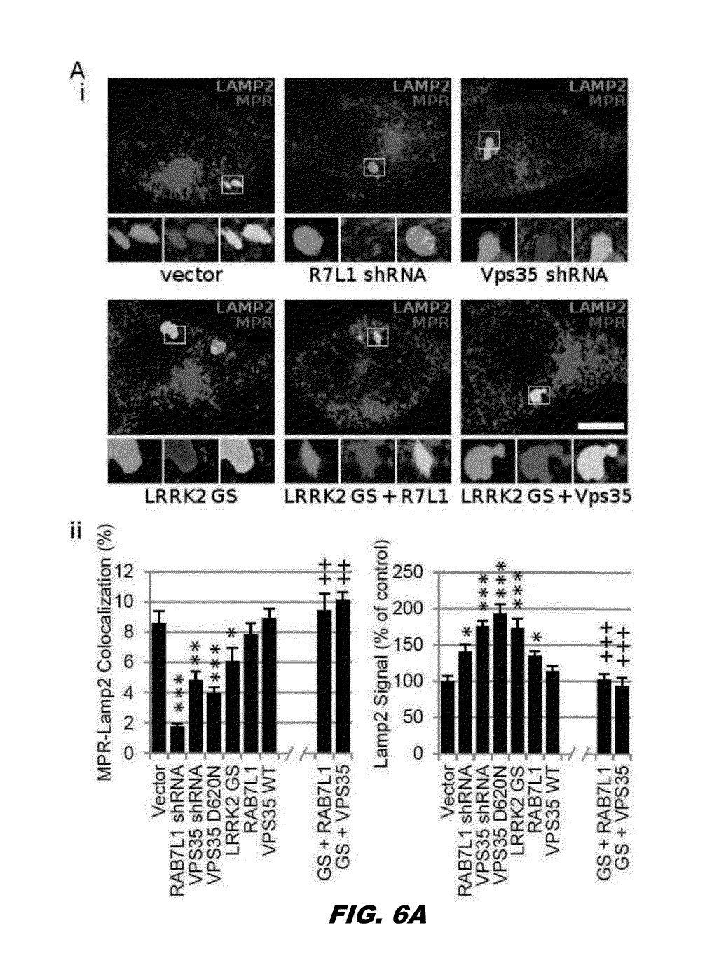

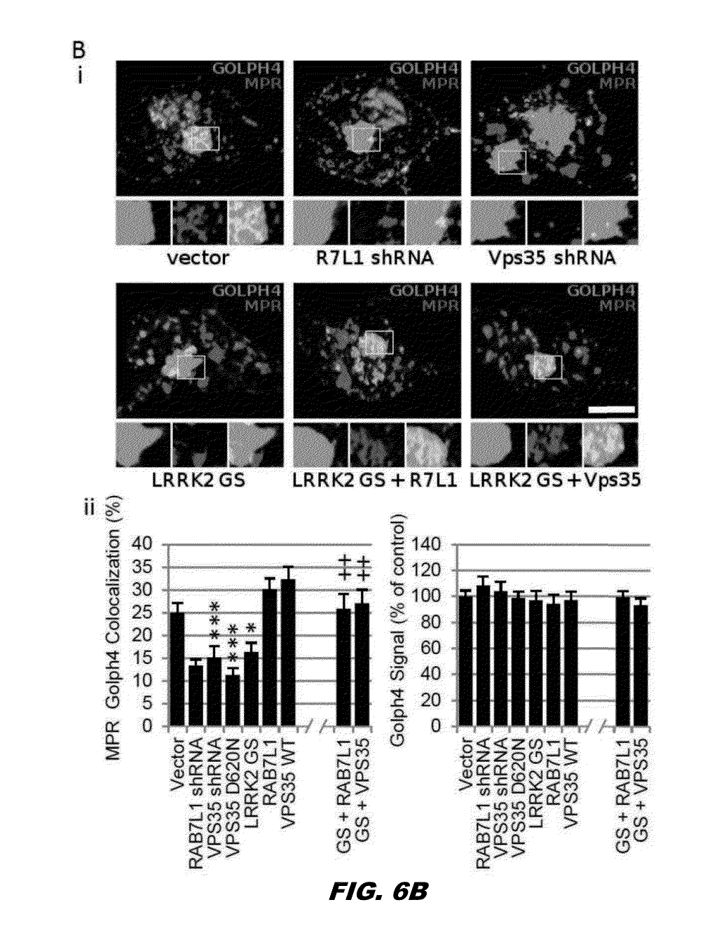

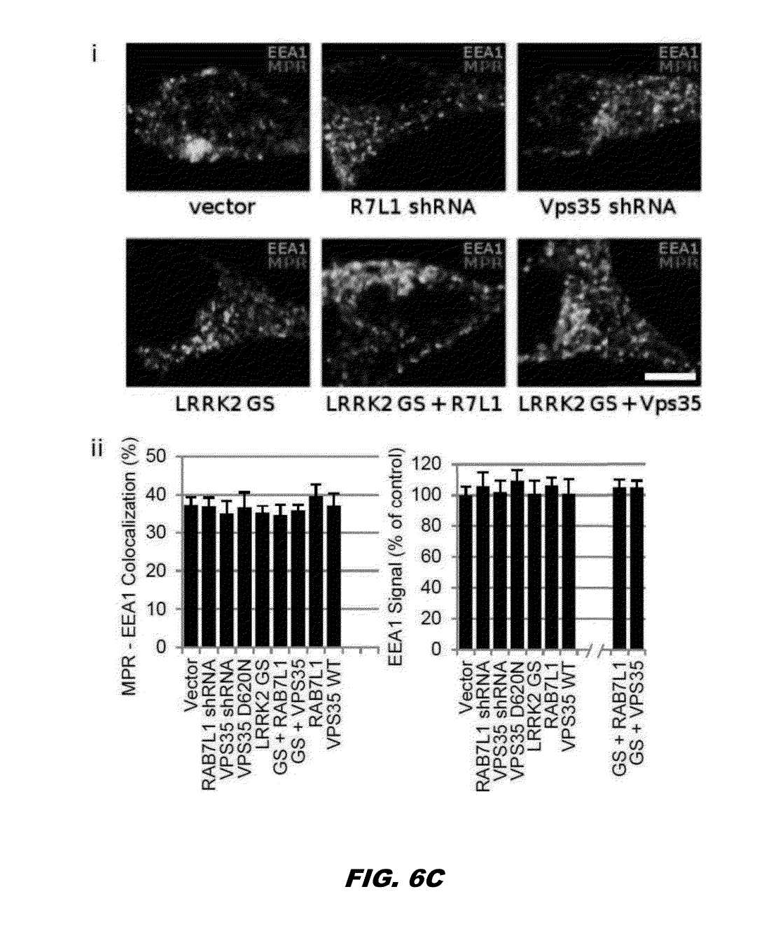

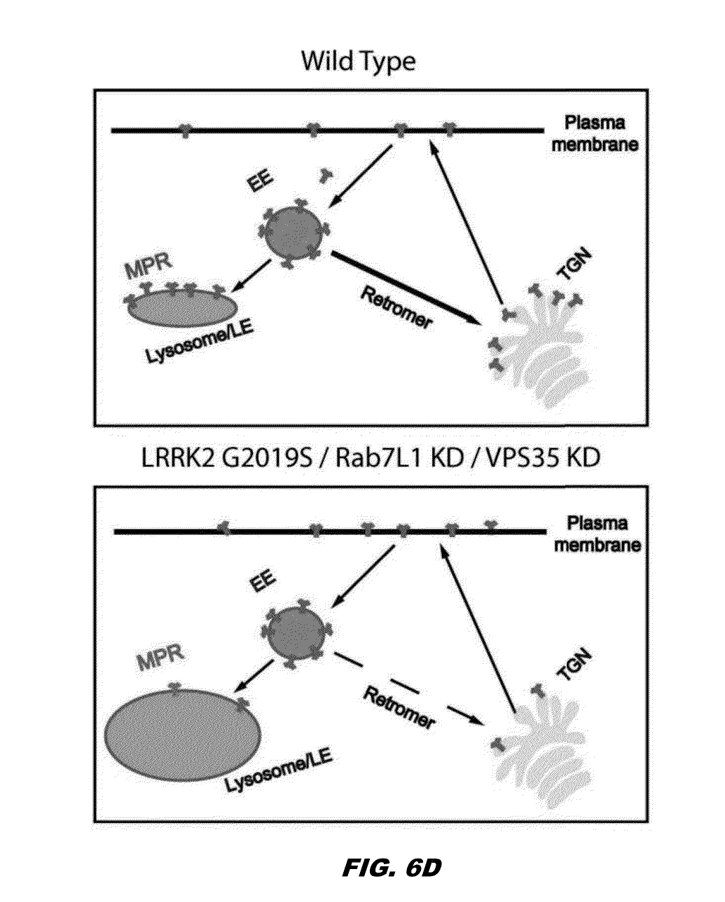

A cellular role for the LRRK2-RAB7L1 pathway was investigated. Prior studies have broadly implicated both of these gene products in intracellular sorting (Sakaguchi-Nakashima et al., 2007; Spano et al., 2011). Expression of the LRRK2 G2019S clinical mutation in rat primary neurons induced lysosomal swelling, as quantified by immunostaining for the lyosomal marker LAMP2 or using the lysosomotropic dye Lysotracker, consistent with prior work and other studies (Dodson et al., 2012; MacLeod et al., 2006; Stafa et al., 2012) (FIG. 6A). In addition to lysosomal enlargement, there was significant reduction in lysosomal accumulation of the cation-independent mannose 6-phosphate receptor (MPR) in terms of the fraction of LAMP2-positive structures stained with MPR. As MPR is required also for the recruitment of lysosomal hydrolases, its deficiency is predicted to lead to functional lysosomal deficits. Knockdown of RAB7L1 was similarly associated with swollen lysosomes and reduced lysosomal MPR, whereas overexpression of RAB7L1 suppressed the lysosomal phenotypes in the context of LRRK2 G2019S expression (FIG. 6A).

MPR is typically recycled between the endolysosome compartment and the Golgi apparatus by the retromer complex (Arighi et al., 2004; Bonifacino and Hurley, 2008; Seaman, 2009; St. George-Hyslop et al., 2009). Given the primary apparent localization of RAB7L1 to the Golgi apparatus (FIG. 3D), as well as the enrichment of LRRK2 at this organelle (FIG. 3D)(Stafa et al., 2012), without being bound by theory, the lysosomal compartment defects described above may be secondary to altered retromer mediated trafficking machinery between these organelles (Bonifacino and Hurley, 2008; Seaman, 2004). Analysis of Golgi structures by immunostaining with the Golph4 marker in primary neurons transfected with either LRRK2 G2019S or shRNA for RAB7L1 did not reveal evidence of gross structural changes, but MPR co-localization at the Golph4-positive Golgi apparatus structures was significantly reduced (FIG. 6B). Accumulation of MPR at early endosomes, assessed by co-staining with the marker early endosomal antigen-1 (EEA1; FIG. 6C), did not appear altered, whereas accumulation at the cell surface appeared increased. The total areas of Golph4, MPR, or EEA staining were unaffected by G2019S LRRK2 expression or RAB7L1 knockdown (FIGS. 6A-C).

The retromer complex is required for retrograde transport of selective cargo—including MPR—between lysosomes and the Golgi apparatus, through endosomal intermediates, in mammalian cells (FIG. 6D) (Bonifacino and Hurley, 2008; St. George-Hyslop et al., 2009), and defects can lead to lysosomal swelling (Arighi et al., 2004). Furthermore, rare mutations in a retromer component, VPS35, were recently linked to rare familial forms of PD (Vilarino-Guell et al., 2011; Zimprich et al., 2011). Knockdown of VPS35 in primary neuron cultures led to reduced MPR co-localization with the Golgi apparatus and with late endosomes/lysosome markers (FIGS. 6A-B), as previously described (Seaman, 2009). Similarly, expression of a familial PD-associated mutation in VPS35, D620N (Vilarino-Guell et al., 2011; Zimprich et al., 2011), phenocopied the MPR missorting phenotype of G2019S mutant LRRK2 expression or VPS35 knockdown (FIGS. 6A-B), suggesting a dominant negative mode of action which is consistent with a predicted structural alteration of a retromer complex interaction motif (Vilarino-Guell et al., 2011; Zimprich et al., 2011). In contrast, overexpression of wild-type VPS35, which promotes trafficking through the retromer pathway, suppressed the altered MPR localization seen with G2019S mutant LRRK2 expression (FIGS. 6A-B). Thus, although it is likely that the LRRK2-RAB7L1 pathway impacts intracellular sorting processes in addition to retromer complex function, suppression of retromer dysfunction is sufficient to rescue the deficits associated with defects in the LRRK2-RAB7L1 pathway.

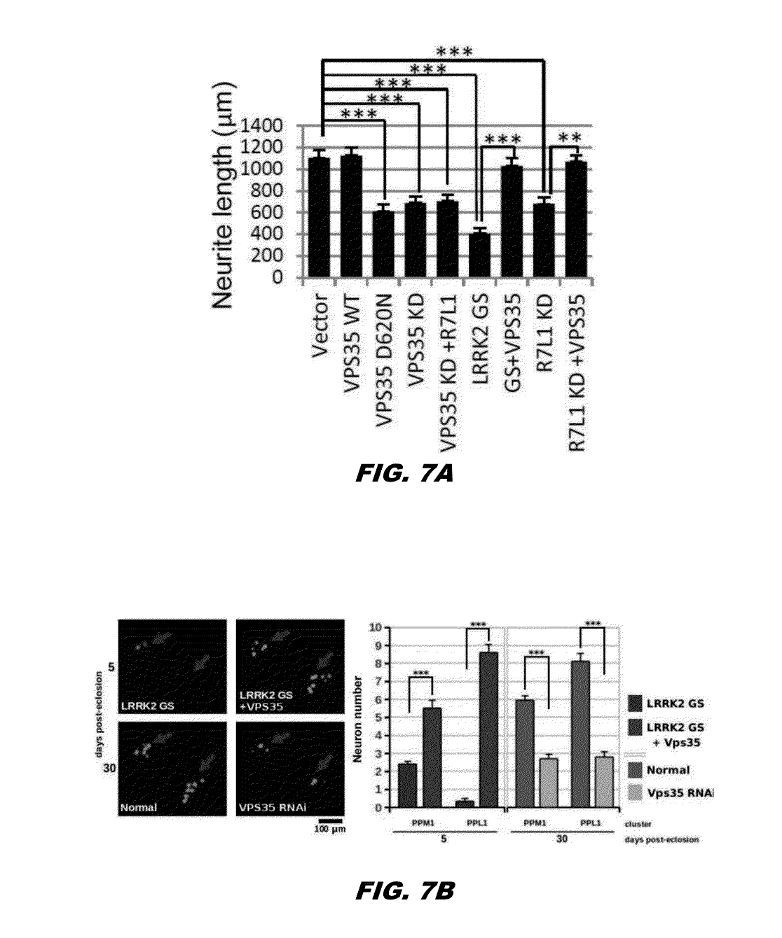

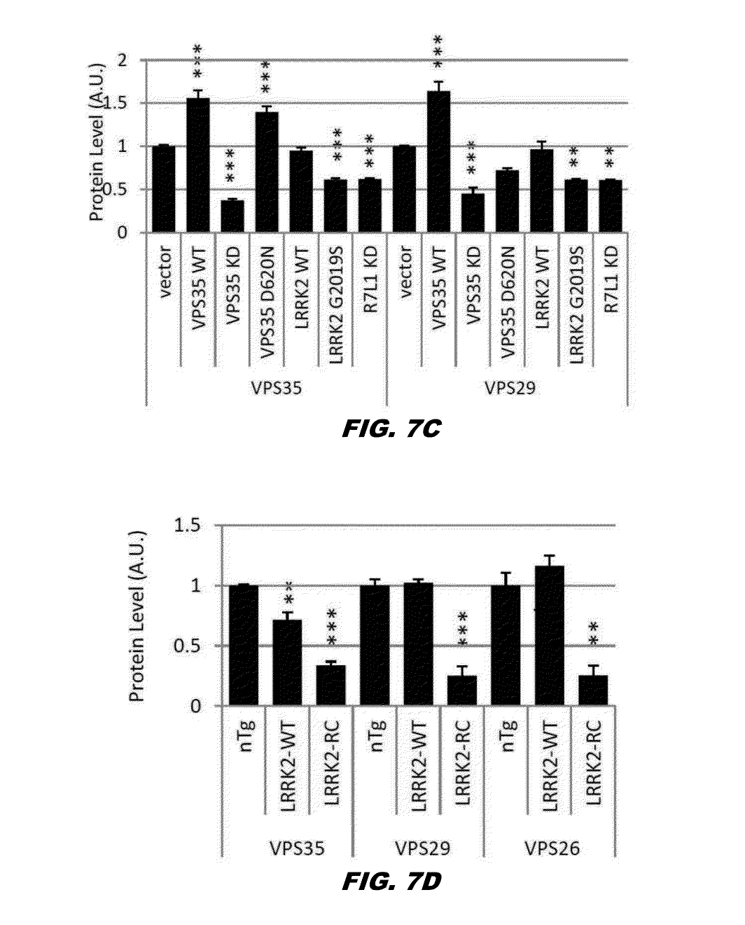

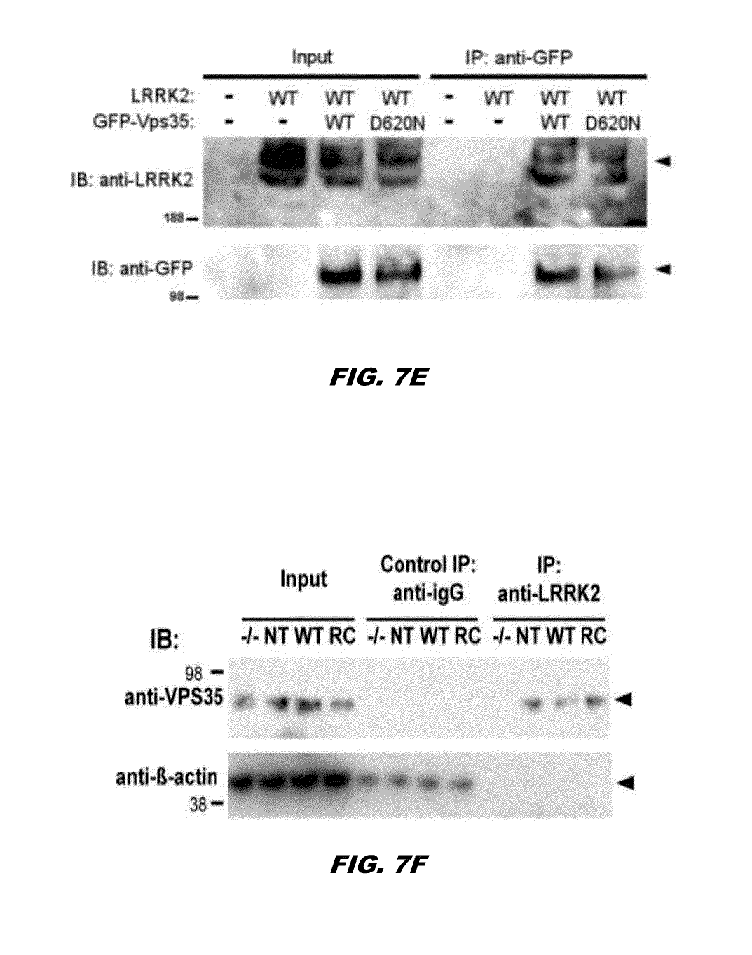

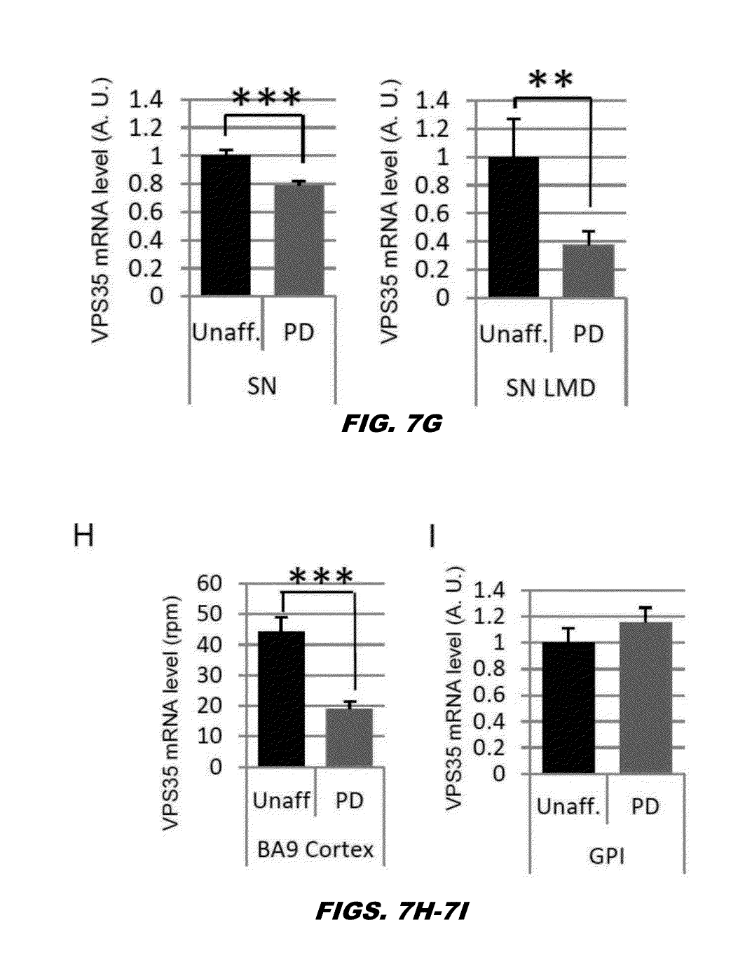

The functional relationship of VPS35 with the LRRK2-RAB7L1 pathway was further investigated in the context of neurite process maintenance. In rat primary neurons, overexpression of VPS35 alone did not directly modify neurite process length, but effectively suppressed the loss of neurite processes in the context of LRRK2 G2019S expression or RAB7L1 knockdown (FIG. 7A). In contrast, knockdown of VPS35 with an shRNA vector, or expression of the VPS35 D620N mutant form, led to neurite process length reduction that phenocopied the effect of LRRK2 G2019S expression. In vivo analysis in the Drosophila CNS further supported a role for retromer dysfunction in the context of LRRK2-RAB7L1 pathway defects. Overexpression of Drosophila VPS35 in Drosophila CNS dopamine neurons rescued the LRRK2 G2019S dopamine neuron loss phenotype (FIG. 7B), and similarly extended the lifespan of G2019S LRRK2 mutant-expressing flies. In contrast, knockdown of VPS35 selectively in Drosophila TH-positive dopamine neurons led to significant cell loss and a reduced lifespan (FIG. 7B).

Reduction in Retromer Complex Component Levels in the Context of LRRK2-RAB7L1 Pathway Defects.

Next potential molecular mechanisms for the apparent defects in retromer pathway function in the context of LRRK2 G2019S mutation or RAB7L1 knockdown were investigated. In mouse N2A neuroblastoma cells, expression of LRRK2 G2019S or knockdown of RAB7L1 led to a significant reduction in the levels of accumulated VPS35 as well as VPS29, a second component of the retromer complex (FIG. 7C). Levels of retromer complex components are dependent on the formation of the entire complex, which also includes VPS29, and thus loss of any complex component is predicted to impact levels of others (Kim et al., 2010). Analysis of transgenic mouse total brain tissue overexpressing the R1441C mutant form of LRRK2 also led to a significant reduction in the accumulation of VPS35 and VPS29, and VPS26 (FIG. 7D).

Although the precise mechanism by which the LRRK2-RAB7L1 pathway impacts retromer complex function and levels remains to be determined, co-immunoprecipitation studies of LRRK2 with VPS35 support a direct interaction between these proteins: Lysates from SH-SY5Y cells co-expressing epitope-tagged V5-LRRK2 (or vector) and eGFP-VPS35 forms, were immunprecipitated for the eGFP tag. Subsequent Western blotting revealed co-purification of LRRK2 with eGFP-VPS35 (FIG. 7E). Similarly, immunoprecipitation of LRRK2 from LRRK2 transgenic mouse brain tissue led to the co-precipitation of endogenous VPS35 (FIG. 7F). It is possible the interactions of LRRK2 with VPS3S and RAB7L1 are within a single complex or multiple complexes.

To relate those findings to sporadic PD, VPS35 levels in PD or unaffected human brain tissue were analyzed. Firsta meta-analysis of substantia nigra (SN) mRNA expression levels in 5 publically available microarray gene expression datasets from patients and controls was carried out (Table 5: totally 144 individuals, 63 unaffected individuals and 81 PD patients), and a highly significant decreased in VPS35 mRNA levels (FIG. 7G) was observed.

TABLE 5
GEO datasets used for the meta-analysis of VPS35 mRNA levels
in SN. Foldchanges and p-values for each individual dataset are indicated.

| | | Re- | Dis- | Fold | | |
|---|---|---|---|---|---|---|
| Dabaset | Probe | gion | ease | change | n | p-vaue |
| GSE26927 | ILMN_21093 | SN | PD | −27% | 20 | 4.90E−03 |
| GSE8397 | 217727_x_at | SN | PD | −10% | 28 | 1.47E−01 |
| GSE7621 | 217727_x_at | SN | PD | −14% | 25 | 1.18E−01 |
| GSE20292 | 217727_x_at | SN | PD | −29% | 29 | 2.80E−04 |
| GSE202923 | 217727_x_at | SN | PD | −9% | 26 | 3.40E−01 |
| GSE20159 | ILMN_1761721 | SN | LBD | −29% | 33 | 3.00E−02 |

Such a decrease was also observed in gene expression data from laser-microdissected PD SN dopamine neurons, when compared to similar cells isolated from unaffected patients (FIG. 7G), as well as in PD cerebral cortex tissue (FIGS. 7H-7I). Taken together, these results support a role for retromer deficiency in the impact of PD-associated genetic risk variants on brain neurons.

### Discussion

Using a brain transcriptomic approach as a starting point, evidence is provided that the impacts of several distinct PD risk-associated common genetic variants are overlapping, even in unaffected PD-free carrier tissue. This points to a convergent pathway of action for such variants. Focusing subsequently on LRRK2 and the PARK16 locus gene RAB7L1, in vitro and in vivo studies support a functional interaction: these gene products bound together and functionally interacted in the regulation of neurite process length in vilro, as well as in the context of dopamine neuron survival in vivo. The impact of LRRK2 and PARK16 variants on brain gene expression was observed even in unaffected carriers of the PARK16 or LRRK2 locus risk variants suggesting the existence of a pre-disease prodromal state in such carriers, that favors subsequent progression.

The most prominent neuronal sorting phenotypes observed in the context of PD-associated LRRK2-RAB7L1 pathway changes were at lysosomes and the Golgi apparatus. Without being bound by theory, the proximal site of action for these proteins may be in defective retromer function at the Golgi apparatus, given the enrichment of both proteins at this structure. Trafficking of MPR to the Golgi apparatus—a function of the retromer complex—is defective, and associated with lysosomal swelling. Although the precise mechanism of retromer dysfunction is unclear, retromer pathway components including VPS35 appear reduced in the context of LRRK2 mutation or RAB711 knockdown. Recently described familial PD-associated clinical mutations in VPS35 phenocopy the deficits associated with LRRK2-RAB7L1 pathway dysfunction, whereas overexpression of VPS35 can rescue such deficits. It is also noted that RAB7 was identified in both the in vitro and in vivo screens of RAB proteins as suppressing the phenotype of LRRK2 mutant pathology, albeit less robustly than RAB7L1. RAB7 is the only RAB protein previously implicated in the regulation of retromer function (Rojas et al., 2008).

Prior studies have supported a role for LRRK2 in vesicular trafficking (Biskup et al., 2006; Dodson et al., 2012; Higashi et al., 2009; MacLeod et al., 2006; Stafa et al., 2012). However, cellular mechanisms of LRRK2 relevant in human brain—and in the context of PD or PD risk variants—have remained unclear. The studies herein are unusual in pursuing a PD genetic pathway using both human brain and model system analyses. A genetic interaction between LRRK2 and RAB7L1 was identified in the context of PD risk, and variants at the loci of these genes impact the brain transcriptome in an overlapping manner. Subsequent cell and animal model studies support a model where LRRK2 and RAB7L1 defects may modify intracellular sorting and retromer pathway function.

It is possible that PD-related defects in LRRK2 and RAB7L1 adversely impact aspects of vesicular trafficking unrelated to retromer function. Nonetheless, inducing retromer function appears sufficient to rescue cellular defects and neuronal survival in these models, suggesting a therapeutic venue in PD patients. It is interesting to note that VPS35 deficits, as well as genetic variants at retromer complex receptor loci such as SORLA (Rogaeva et al., 2007), have also been associated with a second major neurodegenerative disorder, Alzheimer's disease (Muhammad et al., 2008); this suggests a broader role for retromer dysfunction in neurodegeneration. Without being bound by theory, different cargos may be involved in the association of the retromer pathway with distinct pathological processes in Alzheimer's and Parkinson's. To this end, it is of interest to investigate the impact of such retromer dysfunction on aSyn and other proteins associated with PD pathology.

### EXPERIMENTAL PROCEDURE

Drosophila Methods

Drosophila were cultured by standard methods on yeast-cornmeal-agar medium at 25° C. Wild-type and mutant G2019S LRRK2 transgenes were expressed specifically in catecholaminergic neurons, including dopamine neurons, using the Gal4-UAS system described (Fischer et al., 1988). Driver lines used include OK6 (motor neuron), Gmr (eye), G14 (muscle), TH (dopaminergic neuron), and DDC (dopaminergic neuron). A UAS-GFP::nuclear localization sequence (NLS) marker was used to visualize nucleii of cells in which trangenes were expressed (stock 4775 (w 1118; P{UAS-GFP.nls}14), Drosophila Stock Center, Bloomington, Ind.). For the RAB screen, UAS-LRRK2 (G2019S) transgenic Drosophila, crossed with the TH-Gal4 driver, were screened against a UAS-Rab transgenic library (Zhang et al., 2007). Crossings were typically performed using standard balancer chromosome techniques. To generate strains in which the homozygous LRRK2 transgene and another (Driver or marker) transgene lay on the same chromosome (III), genetic recombination was using standard techniques. Adult Drosophila mushroom bodies were dissected as in (Wu and Luo, 2006) and imaged immediately, without fixation, using a Zeiss LSM510 Meta confocal fluorescent microscope. For mortality curves, transgenic Drosophila surviving through adult metamorphosis were counted daily, from the day of pupal eclosion onward. Locomotion deficits were assessed by methods know in the art.

Primary Neurons Culture

Sprague-Dawley rat or mouse PI primary dissociated cortical cultures were prepared and transfected essentially as described (Xia et al., 1996) with the following modifications: cells were plated at high density, approximately 250,000 cells/cm2, in 24-well plates with 500 ul medium/well. Culture medium used for plating cells was Neurobasal-A supplemented with 2% B-27 and 10% FBS. At 1 day after plating, medium was changed to reduced serum (1% FBS+added antimitotic agents: 70 μM uridine and 25 μM 5-fluorodeoxyuridine) and replaced weekly thereafter; for transfections no DMSO was added to the transfection mixture, cells were not subjected to glycerol shock, and a total of 3 μg plasmid DNA was used per well. Cells were fixed in 4% PFA and immunostained with mouse α-RAB7L1 (Santa Cruz, 1:100), and rabbit monoclonal α-LRRK2 (Michal J. Fox Foundation MJFF4, 1:100), then with appropriate fluorescent secondaries (Jackson, 1:1000-2000). Neurite length and neurite puncta (defined as swellings greater than 2 um in diameter) were counted for for at least 20 neurons per condition. Mean puncta number per neuron was normalized to total average neurite length versus wild-type LRRK2 transfected cells. Fluorescent microscopy was performed using a Nikon TE 2000-S microscope and a Zeiss LSM510 Meta confocal microscope. Images were analyzed using Image-Pro Plus (Mediacybernetics) software version 5.1.0.20.

Colocalization Analysis

Primary rat cortical neurons were cultured on glass coverslips, transfected, and fixed as previously described in this methods section. Cells were immunostained for MRP (Abcam #ab2733, 1:400), Golph4(Abcamab #28049, 1:500), Lamp2 (Sigma L0668, 1:500). Fluorescent microscopy was performed using a Zeiss LSM510 Meta confocal microscope. Images were analysed using NIH ImageJ software version 1.45.

Cell Culture, Transfection and Cytochemistry

HEK293T and SH-SY5Y cells were maintained in Dulbecco's modified Eagle's medium (DMEM, Invitrogen) supplemented with 10% fetal bovine serum and 1% penicillin/streptomycin at 37° C. in a 5% CO₂ atmosphere. Transient expression was performed by transfecting the plasmids using Lipofectamine2000 (Invitrogen) according to the manufacturer's instructions. The transfected SH-SY5Y cells grown on glass coverslips for 24 hours were fixed with 4% paraformaldehyde in PBS for 30 minutes, washed three times with PBS and subjected to the observation of fluorescence. For immunostaining of golgi, fixed cells were blocked and permeabilized with PBS containing 0.1% TritonX-100 and 3% bovine serum albumin followed by incubation with anti-Golph4 polyclonal antibody (abcam) and Alexa Fluor 555 goat anti-rabbit IgG (Invitrogen). Staining of nuclei was performed by using SYTOX Orange nucleic acid stain (Invitrogen). Fluorescence was detected using Zeiss LSM 510 confocal microscope.

Immunohistochemistry and Signal Quantification

LRRK2 R1441C or Wt BAC transgenic mice (Li et al., 2009) (Jackson Laboratory) were sacrificed and perfused immediately with 4% PFA for 20 min. Brains were cut by vibratome into sections 60 μm thick. Sections were blocked in 5% NDS overnight at 4 C, then incubated with primary antibodies overnight at 4 C. Antibodies used were sheep monoclonal α-TH (Pelfreeze. 1:500), mouse a-RAB7L1 (Santa Cruz, 1:100), and rabbit monoclonal α-LRRK2 (Michal J. Fox Foundation MJFF4, 1:100). Sections were incubated at room temperature for 2 hours with appropriate fluorescent secondaries (Jackson Laboratories. 1:1000). Microscopy was performed with a Zeiss LSM510 Meta confocal. Fluorescence signal intensity was quantified using NIH ImageJ.

Human Autopsied Brain Samples

Cortical BA9 area brain samples were obtained from the New York Brain Bank and are detailed in Table 5. Anonymous, de-identified tissue from the brain bank was used.

Quantitative Real Time RT-PCR

RT-qPCR was done as described in (Rhinn et al., 2008) RAB7L1 ratio was quantified using ΔΔCt method using primers pairs: RAB7L1_Ex2_fw

(SEQ ID NO: 15)
(CAGCAAACACTACAAGTCCACG)

and RAB7L1_Ex3_rv

(SEQ ID NO: 16)
(CAGCTGAAGCCGCACTATCTCG);

RAB7L1_Ex4_fw

(SEQ ID NO: 17)
(GACAGCAAGCTCACACTACCCA);

RAB7L1_Ex5_rv

(SEQ ID NO: 18)
(TCTGTCCAACCTGTGAAACCGT)

for human brain samples.

Minigene Splicing Assay

The human SH-SY5Y neuroblastoma cell line (ATCC) was cultured following ATCC's instructions, plated at densities of 4.10e5 cells per well (48-well plates) in wells coated with 0.1% gelatin (Specialty Media, Millipore) 24 hours prior to transfections. Transfections were performed with Lipofectamine 2000 reagent (Invitrogen) following manufacturer's instructions. After transfection with plasmids encoding the reporter contruct, RNA was extracted using miRNeasy kit (Qiagen) and reverse transcribed using Superscript III reverse transcriptase (Invitrogen) following manufacturer's instructions. The eDNA was amplified by PCR using the following primers:

(SEQ ID NO: 19)
GGAGGGCGTCTAGGGAATCGAG

(Fw, complementary to exon1 of RAB7L1) and

(SEQ ID NO: 20)
CTTCAGGGTCAGCTTGCCGTAG

(Rev., complementary to GFP CDS) and Accuprime high-fidelity polymerase (Invitrogen) following manufacturer's instruction with an hybridization at 55 C and an elongation step of 1 min. Pictures from an ethidhium bromide stained agarose gels of the migrated PCR products was analyzed using ImageJ software.

Supplementary Experimental Procedures

Western Blots

Mouse brain protein fractions were prepared as follows. Mouse striata were dissected and homogenized by motorized dounce in Krebs-Ringer buffer with 0.32 M sucrose, then centrifuged at 3000×g for 10 min. Supernatant was collected and centrifuged at 10,000×g for 30 min. Pellet was resuspended in NuPage loading buffer (Invitrogen). Human brain proteins were prepared from frozen blocks using RIPA reagent (Pierce) following manufacturer's instruction. SDS-Page and Western Blot were performed according to manufacturer's protocols with NuPage Bis-Tris Mini Gel and Xcell II Blot Module (Invitrogen). Antibodies used include: LRRK2 (MJFF #1 & #2, 1:200), Rab5 (Abcam ab18211, 1:500), RAB7L1 (clone 2B8, Sigma, 1:400, clone 31-E, Santa Cruz sc-81924, 1:400), anti-Flag M2 (Sigma, 1:1000), anti-GFP (Covance, 1:1000), anti-alpha-tubulin (DM1A, 1:2000), SNAP25 (Abcam ab41455, 1:500), VAMP2 (Abcam ab3347, 1:500), beta-actin (clone C4) (Abcam ab3280, 1:800) and appropriate HRP-conjugated secondaries (Jackson, 1:2000). Blots were visualized using Supersignal luminol substrate (ThermoScientific #34075).

Immunoprecipitation

For cultured cells, HEK293T cells transfected for 48 hours were lysed with lysis buffer containing 0.5% Triton-X. 1 mM EDTA and protease inhibitor cocktail (Sigma). The lysates were rotated at 4° C. for 1 hr followed by centrifugation at 20,000 g for 5 min. The supernatant was added to 30 ul (slurry volume) of Dynabeads protein G (Invitrogen) preincubated without (preclear) and with an anti-flag M2 monoclonal antibody (Sigma) and the mixture was rotated for 30 min at 4° C. The beads were washed three times with ice-cold PBS and subjected to immunoblotting.

For mouse tissue, whole brains were dissected and homogenized by motorized dounce at 0° C. in Invitrosol detergent (Invitrogen) and Ix Protease inhibitor cocktail (Pierce) according to manufacturer's protocol Lysate was incubated shaking for 30 min at 4° C. with gel beads (Pierce Co-IP kit #26149) covalently conjugated to LRRK2 antibody (either MJFF #2 or #4) or a control IgG antibody. Beads were washed 4×10 min each, and then eluted. Eluant was analyzed by Western Blot, probed for LRRK2 (MIFF #1, 1:200), Rab7L1 (Santa Cruz sc-81924, 1:400), Rab11 (Abcam ab3612, 1:400), and beta-actin (clone C4) (Abcamab3280, 1:600).

DNA Constructs

The plasmid encoding rat RAB7L1 cDNA sequence was purchased from Open Biosystems, and the sequence was digested and ligated into BgIII-EcoRI site of pEGFP-C1 expression vector (Clontech) to generate N-terminally GFP-tagged RAB7L1. As the purchased RAB7L1 sequence contained 286 bp insertion in the middle of cDNA resulting in the generation of stop codon, this insertion was removed by a long-PCR protocol. The plasmids encoding constitutive active (Q67L) and dominant negative (T2 IN) rat RAB7L1 were generated by using site-directed PCR-mutagenesis kit (Stratagene) from the plasmid encoding N-terminally GFP-tagged wild-type RAB7L. All sequences were verified by DNA sequencing. Plasmids encoding wild-type and mutant Rab7 constructs were from Addgene; Rab3 and Rab5 constructs were also used. Plasmids encoding full-length human LRRK2 (wild-type, G2019S, K1906M) tagged with 3×FLAG at the N-terminus were used. Splice reporter minigene bearing plasmid was created by insertion of a synthesized sequence corresponding to the first exon, the first intron and the second exon and 200 bp of the second intron of human RAB7L1 gene in a pEGFP-N1 vector (Clontech) between its Xhol and HindIII restriction sites. Rab7L1 shRNA plasmid came from Sigma (MISSION shRNA clone NM_144875). LRRK2 plasmids used were those published (MacLeod et al., 2006), and confirmed.

polyA-RNAseq

Library generation and sequencing: First-strand cDNA was synthesized from 1 μg of RNA per biological sample using SuperScript III (Invitrogen) following manufacturer's instructions and using the pdT-FS oligonucleotide to prime the reverse transcription. Barcoded first-strand samples from different samples were then pooled and treated with RNase H (Invitrogen) at 37° C. for 20 minutes followed by 15 minutes at 75° C. to degrade RNA template. First-stand cDNA was then purified using QIAquick PCR Purification kit (Qiagen) in a total volume of 30 uL. Second-strand cDNA was synthesized from 25 uL of first-strand cDNA template by adding 10 μl 10× buffer 2 (NEB), 5 μl 10 mM dNTPs, 20 U Klenow Fragment (3′→5′exo-; NEB), 10 μl of 100 μM tagged 2nd strand primer (R-SS oligonucleotide:

5′-TCCGATCTGANNNNNNN-3′

with N=A,C,T,G mix (SEQ ID NO: 21)) and 46 μl water. The reaction mix was incubated at 37° C. for 30 minutes, followed by 10 minutes at 75° C. then cooled down at 4° C. Double-stranded cDNA was purified using PureLink PCR micro columns (Invitrogen) in a 30 uL volume. Illumina-compatible libraries were then generated by PCR from 25 uL of double-stranded cDNA template using Accuprime Pfx polymerase (Invitrogen) following manufacturer's instruction with NNSR forward

(SEQ ID NO: 22)
(5′-AATGATACGGCGACCACCGAGATCTACACTCTTTCCCTACACGACG
CTCTTCCGATCTC-3′)

and NNSR reverse

(SEQ ID NO: 23)
(5′-CAAGCAGAAGACGGCATACGAGATCGGTCTCGGCATTCCTGCTGAA
CCGCTCTTCCGATCTGA-3′)

primers. Thermo-cycling conditions were 2 min at 94° C. followed by 2 cycles of 94° C. for 10 s, 40° C. for 2 min, 68° C. for 1 min; 8 cycles of 94° C. for 10 s, 60° C. for 30 s, 68° C. for 1 min; 15 cycles of 94° C. for 15 s, 60° C. for 30 s, 68° C. for 1 min with an additional 10 s added at each cycle; and 68° C. for 5 min before cooling to 4° C. Amplified libraries were purified using PureLink PCR micro columns (Invitrogen) and directly used to generate clusters for sequencing-by-synthesis using the Illumina HiSeq 2000 platform. 100 bp single-end reads were obtained by sequencing to generate more than 300 million reads for the 34 samples.

Data was analyzed using Galaxy (Goecks et al., 2010): Illumina reads were converted to FASTQ Sanger format using FASTQ Groomer, the first 27 bp at their 5′ends of the reads were trimmed using FASTQ Trimmer to remove the polyA and adapters sequences and mapped to human hg19 genome using Burrows-Wheeler Alignment tools (Langmead et al., 2009) with Galaxy's default settings allowing 4% of missing alignments. All those tools are included in the Galaxy NGS toolset.

Human Sample Genotyping

DNA was extracted from brain samples using DNeasy kit (Qiagen) and amplified by PCR using primers RAB7L1_Genot_fw

(SEQ ID NO: 24)
(GGTGAGCCTCCGCACTCG)

and RAB7L1_Genot_rv

(SEQ ID NO: 25)
(TTCCCACCCACCGCCTGT)

and Accuprime polymerase (Invitrogen) following manufacturer's instruction with an hybridization at 55° C. and an elongation step of 1 min. PCR products were purified using PureLink PCR columns (Invitrogen) submitted to Sanger sequencing (GeneWiz, NJ) using RAB7L1_Genot_fw primer and analyzed using SeqScanner (Applied Biosystems).

GWAS Epistasis Analysis

The AJ GWAS dataset included a total of 278 cases and 178 controls that were genotyped using the Illumina Human 610-quad bead arrays (Cases n=91 and controls n-96) or the Illumina Human 660-quad bead arrays (Cases n=191 and controls n=84). Details of the genotyping and quality control assessments are provided in Liu et al (2011). Subjects were participants in two studies the Genetic Epidemiology of Parkinson Disease and the AJ study. Ascertainment and a description of the study participants is provided in detail in Marder et al (Marder et al., 2003) and Liu et (Liu et al., 2011). All subjects in the GWAS AJ dataset were genotyped for the LRRK2 G2019S mutation and for GBA mutations common in the AJ population (N370S, L444P, 84insGG, IVS2+1 G>A, V394L, R496H, D409H, A456P and V460V). For the epistasis analysis LRRK2 and GBA mutation carriers were removed from the dataset. The final dataset is comprised of 239 PD cases and 178 controls.

In addition to the AJ dataset, 3 additional publicly available GWAS datasets were studied and downloaded from NCBI's dbGap repository (Mailman et al., 2007): NGRC (CIDR/NGRC Genes and Environment, dbGap phs000196.v2.p1, (Hamza et al., 2010)), that comprises 2013 cases and 1995 controls, NINDS (NINDS-Genome-Wide Genotyping in Parkinson's Disease, dbGap phs000089.v1.p1, (Fung et al., 2006)) that comprises 267 cases and 270 controls and Mayo (Mayo-Perlegen LEAPS (Linked Efforts to Accelerate Parkinson's Solutions) Collaboration, dbGap phs000048.v1.p1, (Maraganore et al., 2005)) that comprises 443 cases and 443 controls. All subsequent genetic analysis were done using gplink (Purcell et al., 2007) software. Epistasis between PARK16 (rs823114, as identified to modify PD age of onset in LRRK2 mutations carriers (Latourelle et al., 2011)) and LRRK2 (rs11176052 as identified by PD GWAS meta-analysis (Lill et al., 2012)) in each dataset was evaluated using epi function. As those SNPs were not present in the lower density Perlgen array used in the Mayo datasets, their best proxies were determined using SNAP in the 1000 genomes CEU population (see below); rs823154 and rs11174928 were used for the analysis in this dataset for PARK16 and LRRK2 respectively. Odd-ratios and their confidence intervals were evaluated using asso function. Meta-analysis and combination of p-values were done using Metal (http://www.sph.umich.edu/csg/abecasis/metal/).

SNP Selection

SNP-SNP pairwise linkage disequilibrium was assessed by SNAP phase (Johnson et al., 2008) using the CEU population panel from the 1000 genomes and HapMap dataset. PD associated SNPs were evaluated based on PDGene meta-analysis results (Lill et al., 2012).

GPI Analysis

Genome-wide SNP variant and gene expression data for 364 individuals were previously described (Myers et al., 2007). Normalized data corrected for covariates such as age, sex and batch effects were processed using R for gene expression analysis and gplink (Purcell et al., 2007) for genotypes. Subsequently for a given SNP, Pearson's correlation coefficient is calculated between the expression level of each gene (within the whole transcriptome dataset) and the allele load across the panel of samples. Associations were arbitrarily described with the high-risk variant at any given disease-associated SNP with positive values, and with the protective low-risk variant with negative values. As a consequence, a gene whose expression is consistently higher in samples from individuals who carry the disease-associated high-risk variant (relatively to the expression in the context of the protective low-risk variant) across the entire sample set will show a positive correlation coefficient (such as Gene 1 in FIG. 12B). By assessing the correlation coefficients across the entire transcriptome for a given variant, the GPI can be obtained.

Formally, the GPI for a SNP is a n-vector of numerical values between −1 and 1, where n is a number of genes whose expression levels is available, and corresponds to the collection of the expression level correlation with the allelic load for each individual gene. The GPI of SNPx was thus calculated as

$$GPISNPx = [ \begin{matrix}r ( {A}_{{G}_{i}} , {L}_{{SNP}_{x}} ) \\ … \\ … \\ \begin{matrix}… \\ r ( {A}_{{G}_{n}} , {L}_{{SNP}_{x}} )\end{matrix}\end{matrix} ]$$

with

$${A}_{{G}_{i}} = [ \begin{matrix}{( {a}_{{G}_{i}} )}_{1} & ⋯ & {( {a}_{{G}_{i}} )}_{p}\end{matrix} ] ,$$

expression level of gene i across all samples, (aGi)ⱼ being the expression level of gene i in sample j

$${L}_{{SNP}_{x}} = [ \begin{matrix}{( {l}_{{SNP}_{x}} )}_{1} & ⋯ & {( {l}_{{SNP}_{x}} )}_{p}\end{matrix} ] ,$$

high-risk allele load of SNP x across all samples (I_{SNP}x) being the high-risk allele load of SNP x in sample j.

$${( {l}_{{SNP}_{x}} )}_{1} = \{ \begin{matrix}0 & if j is homozygous for the low risk allele of SNPx \\ 1 & if j is heterozyguous for SNPx \\ 2 & if j is homozygous for the high risk allele of SNPx\end{matrix}$$

(See FIG. 12A). (AG1, LSNPx) is the Pearson correlation coefficient between the expression level of gene 1 and the disease-associated allelic load of SNPx across all samples. (A, LSNPx) is positive for genes whose expression levels are increased in the presence the risk allele and negative for genes whose expression levels are decreased (Genes 1 and 3 respectively in FIG. 12B).

The intersection between the GPIs calculated for two SNPs is evaluated in a threshold-free approach by considering all the genes which show a correlation in the same direction for different GPIs. Formally, with GPI_{SNPxX SNPy}=GPI_{SNPx}∩GPI_{SNPy}:

$${GPI}_{{SNP}_{x} \times {SNP}_{y}} [ k ] = \{ \begin{matrix}\frac{{GPI}_{{SNP}_{x}} [ k ] + {GPI}_{{SNP}_{y}} [ k ]}{2} & if {GPI}_{{SNP}_{x}} [ k ] , {GPI}_{{SNP}_{y}} [ k ] > 0 \\ 0 & if {GPI}_{{SNP}_{x}} [ k ] , {GPI}_{{SNP}_{y}} [ k ] < 0\end{matrix}$$

In order to study potential interactions between two SNPs, a quantitative trait was defined for which classical genetic interaction analysis could be applied.

A genetic interaction is broadly defined as when the combined phenotypic effect of two mutations (in distinct genes) is not equal to the sum of the two individual phenotypic effects. Thus, such a non-additive interaction can either represent synergy (the combined effect is greater than the sum of its parts) or occlusion (the combined effect is less than the sum of its parts). The prediction for an occlusive genetic interaction is that the transcriptome effect of a risk allele at either one of the 2 genes will preclude the effect of a second risk allele.

A quantitative trait phenotype was defined for the classical genetic interaction analysis. This is most simply done by examining gene expression values that are highly impacted in common by the 2 SNPs individually (as identified above by the GPI intersection genes), and then querying the effect of their combination. Without being bound by theory, any of the gene expression values from the GPI intersection could be queried for a genetic interaction. Rather than querying individual genes expression phenotypes, a single scalar value was generated that represents the combined effect on the expression patterns of all of the relevant genes (as defined by the GPI analysis above; we used the genes most significantly impacted with p<0.01, empirically assessed by resampling). To compute this scalar value, termed the expression quantitative trait (eQT), a standard linear algebra manipulation was used: the combined quantitative trait is the sum of the expression levels of the selected genes, weighted by their GPI intersection coefficients (which reflects how consistently and strongly they are affected by both SNPs).

Formally, the complex expression phenotype for sample j will be:

$${( {CEP}_{xy} )}_{j} = {\sum }_{m = 1}^{M} {GPI}_{{SNP}_{x}} \times {SNP}_{y} [ {t}_{m} ] \cdot {( {a}_{{G}_{{t}_{m}}} )}_{j}$$

with a selection of M genes from GPI_{sNPxX SNPy} and tm the position in the GPIs vector of the m^{th} highest absolute value in GPI_{sNPxX SNPy}.

To actually perform the interaction analysis, we first determine the genotypes for the two SNPs of interest and then proceeded to linear model regression for the quantitative trait across all samples. Computationally, the effect of both SNPs on the quantitative phenotype was assessed using R lm function, by fitting of the linear model CEP_{xy}˜x0+x1·L_{SNPx}+x2·L_{SNPy}+x3·L_{SNPx}L_{SNPy}. The test for interaction is based on the significance associated with the coefficient x₃.

Disease Association Expression Profiles

For a given disease dataset, Pearson's correlation coefficient is calculated between the expression level of each gene (within the whole transcriptome dataset) and the disease phenotype across the panel of samples. Associations with the disease were arbitrarily described as positive values, consistently with the orientation assigned in the GPI calculation to the high-risk allele load of a disease-associated SNP. As a consequence, a gene whose expression is consistently higher in disease samples than in unaffected will show a positive correlation coefficient. By assessing the correlation coefficients across the entire transcriptome for a given variant, a global disease profile (GDP) can be obtained.

Formally, the GDP produces an object of the same class as the GPI, a n-vector of numerical values between −1 and 1, where n is a number of genes whose expression levels is available, and corresponds to the collection of the expression level correlation with the disease phenotype (0 for unaffected, 1 for disease) across the samples. Formally,

$$G D P D = [ \begin{matrix}r ( {A}_{{G}_{1}} , {P}_{D} ) \\ … \\ … \\ \begin{matrix}… \\ r ( {A}_{{G}_{n}} , {P}_{D} )\end{matrix}\end{matrix} ]$$

with PD=[(P_{D})1 . . . (P_{D})p], binary phenotype associated to disease D across all samples, (PD)j being the disease-associated binary phenotype in sample j.

$${( {l}_{SNPx} )}_{j} = \{ \begin{matrix}0 & if j is unaffected \\ 1 & if j is affected\end{matrix}$$

and r(A_{G1}, P_{D}) the Pearson correlation coefficient between the expression level of gene i and the disease-phenotype across all samples. As a consequence, a gene whose expression level is increase in the course of the disease will be associated to a positive correlation in the disease expression profile. When multiple Affymetrix probesets were available for a given gene, the probeset showing the highest expression level was systematically selected. For resampling procedures, the values of the phenotype vector are randomly reattributed to the different samples (label switch).

Datasets of normalized gene expression (accession numbers GSE20168, GSE7621, GSE1297, GSE3790, GSE12654) were downloaded from the Gene Expression Omnibus website (http://www.ncbi.nlm.nih.govigeo/).

Similarity between a disease expression profile and a GPI intersection is evaluated by the Pearson correlation between the GPI intersection and the GDP across the subset of genes that show the highest absolute value in the GPI intersection. (In the case of the 7 PD SNPs intersection, the top 135 genes out of the 352 non-null were selected as a core set, with a FDR<5% based on resampling analysis of a 7 SNPs GPI intersection size). All data manipulations and analysis were done using R.

SNP within Probes

Common SNPs within the target sequences of microarray probes have indeed emerged as a potential technical issue for eQTL analysis in recent years (Alberts et al., 2007; Chen et al., 2009), due to the mis-hybridization caused by the allelic variant of such a SNP that does not match the designed target sequence; as a consequence of the poorer hybridization of the probe to its target sequence, the amount of target sequence might be under-evaluated. Classical eQTL studies aim at identifying relationships between the allelic load of a given variant (herein called “studied variant”) and the level of expression of a given gene. In the case of such eQTL studies, results can be biased if the allele leading to a poorer hybridization segregates with one allele of the studied SNP. This most often happens for cis-eQTL, as the local physical structure of the chromosome can lead to a systemic segregation between the studied variant and the one within the probe if those are in linkage disequilibrium. The GPI analysis can be seen as a globalization at a transcriptome-wide scale of eQTL studies, where the effect of a studied variant is considered on transcriptome-wide gene expression levels in a single measurement. As all the genes are considered with the same weight, there are two direct implications to that 1) the global measurement should be robust to potential technical issues (such as SNP-in-Probe) affecting a single probe, as this will only affect a fraction of a percent of the total GPI signal 2) the GPI is mostly based on trans-effect measurements, as more than 99% of the genes will be considered as “trans” by reference to any studied variant.

To empirically support the robustness of the GPI to SNPs in probes, all the probes sequences used in the study were obtained from Illumina, and were mapped on the human genome using Burrows-Wheeler Alignment (Langmead et al., 2009) to identify those that target sequences containing a SNP whose minor allele frequency is superior to 5% in HapMap Caucasian populations (Consortium, 2003). The whole analysis was then reproduced by excluding the 272 probes satisfying those criteria, and the same results were obtained as the one presented in the manuscript in FIGS. 1C and D, establishing the robustness of the GPI procedure, based on its transcriptome-wide design and ruling out any significant effect caused by the presence of SNPs within the probes.

### REFERENCES

Abeliovich, A., and Flint Beal. M. (2006). Parkinsonism genes: culprits and clues. J Neurochem 99, 1062-1072.
Abeliovich, A., Schmitz, Y., Farinas. I., Choi-Lundberg, D., Ho, W. H., Castillo, P. E., Shinsky, N., Verdugo, J. M., Armanini, M., Ryan, A., et al. (2000). Mice lacking alpha-synuclein display functional deficits in the nigrostriatal dopamine system. Neuron 25, 239-252.
Andres-Mateos, E., Mejias, R., Sasaki, M., Li, X., Lin, B. M., Biskup, S., Zhang, L., Banerjee, R., Thomas. B., Yang, L., et al. (2009). Unexpected lack of hypersensitivity in LRRK2 knock-out mice to MPTP (1-methyl-4-phenyl-1,2,3,6-tetrahydropyridine). J Neurosci 29, 15846-15850.
Arighi, C. N., Hartnell, L. M., Aguilar, R. C., Haft, C. R., and Bonifacino, J. S. (2004). Role of the mammalian retromer in sorting of the cation-independent mannose 6-phosphate receptor. J Cell Biol 165, 123-133.
Berger, K. L., Cooper, J. D., Heaton, N. S., Yoon, R., Oakland. T. E., Jordan, T. X., Mateu, G., Grakoui, A., and Randall, G. (2009). Roles for endocytic trafficking and phosphatidylinositol 4-kinase III alpha in hepatitis C virus replication. Proc Natl Acad Sci USA 106, 7577-7582.
Biskup, S., Moore, D. J., Celsi, F., Higashi, S., West, A. B., Andrabi, S. A., Kurkinen, K., Yu, S. W., Savitt, J. M., Waldvogel, H. J., et al. (2006). Localization of LRRK2 to membranous and vesicular structures in mammalian brain. Ann Neurol 60, 557-569.
Bonifacino, J. S., and Hurley, J. H. (2008). Retromer. Curr Opin Cell Biol 20, 427-436.
Cooper, A. A., Gitler. A. D., Cashikar. A., Haynes, C. M., Hill, K. J., Bhullar. B., Liu, K., Xu, K., Strathearn, K. E., Liu. F., et al. (2006). Alpha-synuclein blocks ER-Golgi traffic and Rab1 rescues neuron loss in Parkinson's models. Science 313, 324-328.
Dietzl, G., Chen, D., Schnorrer, F., Su, K. C., Barinova, Y., Fellner, M., Gasser, B., Kinsey, K., Oppel, S., Scheiblauer, S., et al. (2007). A genome-wide transgenic RNAi library for conditional gene inactivation in Drosophila. Nature 448, 151-156.
Dodson, M. W., Zhang, T., Jiang, C., Chen, S., and Guo, M. (2012). Roles of the Drosophila LRRK2 homolog in Rab7-dependent lysosomal positioning. Hum Mol Genet 21, 1350-1363.
Feany. M. B., and Bender, W. W. (2000). A Drosophila model of Parkinson's disease. Nature 404, 394-398.
Fischer, J. A., Giniger, E., Maniatis, T., and Ptashne, M. (1988). GAL4 activates transcription in Drosophila. Nature 332, 853-856.
Friggi-Grelin, F., Coulom. H., Meller, M., Gomez, D., Hirsh, J., and Birman, S. (2003). Targeted gene expression in Drosophila dopaminergic cells using regulatory sequences from tyrosine hydroxylase. Journal of neurobiology 54, 618-627.
Gan-Or, Z., Bar-Shira, A., Dahary, D., Mirelman, A., Kedmi. M., Gurevich, T., Giladi, N., and Orr-Urtreger, A. (2012). Association of sequence alterations in the putative promoter of RAB7L1 with a reduced parkinson disease risk. Arch Neurol 69, 105-110.
Grill, B., Bienvenut, W. V., Brown, H. M., Ackley, B. D., Quadroni, M., and Jin, Y. (2007). C. elegans RPM-1 regulates axon termination and synaptogenesis through the Rab GEF GLO-4 and the Rab GTPase GLO-1. Neuron 55, 587-601.
Hamza, T. H., Zabetian, C. P., Tenesa, A., Laederach, A., Montimurro, J., Yearout, D., Kay, D. M., Doheny, K. F., Paschall, J., Pugh, E., et al. (2010). Common genetic variation in the HLA region is associated with late-onset sporadic Parkinson's disease. Nat Genet 42, 781-785.
Hardy, J., Cai, H., Cookson, M. R., Gwinn-Hardy, K., and Singleton, A. (2006). Genetics of Parkinson's disease and parkinsonism. Ann Neurol 60, 389-398.
Heo, H. Y., Kim, K. S., and Seol, W. (2010). Coordinate Regulation of Neurite Outgrowth by LRRK2 and Its Interactor, Rab5. Exp Neurobiol 19, 97-105.
Hermann, G. J., Schroeder, L. K., Hieb, C. A., Kershner, A. M., Rabbitts, B. M., Fonarev, P., Grant, B. D., and Priess, J. R. (2005). Genetic analysis of lysosomal trafficking in Caenorhabditis elegans. Mol Biol Cell 16, 3273-3288.
Higashi, S., Moore, D. J., Yamamoto, R., Minegishi, M., Sato, K., Togo, T., Katsuse, O., Uchikado, H., Furukawa, Y., Hino, H., et al. (2009). Abnormal localization of leucine-rich repeat kinase 2 to the endosomal-lysosomal compartment in lewy body disease. J Neuropathol Exp Neurol 68, 994-1005.
Kim, E., Lee, Y., Lee, H. J., Kim, J. S., Song, B. S., Huh, J. W., Lee, S. R., Kim, S. U., Kim, S. H., Hong, Y., et al. (2010). Implication of mouse Vps26b-Vps29-Vps35 retromer complex in sortilin trafficking. Biochem Biophys Res Commun 403, 167-171.
Lang, A. E., and Lozano, A. M. (1998). Parkinson's disease. First of two parts. N Engl J Med 339, 1044-1053.
Latourelle, J. C., Hendricks, A. E., Pankratz, N., Wilk, J. B., Halter, C., Nichols. W. C., Gusella, J. F., Destefano, A. L., Myers, R H., and Foroud, T. (2011). Genomewide linkage study of modifiers of LRRK2-related Parkinson's disease. Mov Disord 26, 2039-2044.
Lee, S. B., Kim, W., Lee, S., and Chung, J. (2007). Loss of LRRK2/PARK8 induces degeneration of dopaminergic neurons in Drosophila. Biochem Biophys Res Commun 358, 534-539.
Li, Y., Liu, W., Oo, T. F., Wang, L., Tang, Y., Jackson-Lewis, V., Zhou, C., Geghman, K., Bogdanov, M., Przedborski, S., et al. (2009). Mutant LRRK2(R1441G) BAC transgenic mice recapitulate cardinal features of Parkinson's disease. Nat Neurosci 12, 826-828.
MacLeod, D., Dowman, J., Hammond, R., Leete, T., Inoue. K., and Abeliovich, A. (2006). The familial Parkinsonism gene LRRK2 regulates neurite process morphology. Neuron 52, 587-593.
Montgomery, S. B., Sammeth, M., Gutierrez-Arcelus, M., Lach, R. P., Ingle, C., Nisbett, J., Guigo, R., and Dermitzakis, E. T. (2010). Transcriptome genetics using second generation sequencing in a Caucasian population. Nature 464, 773-777.
Muhammad. A., Flores, I., Zhang, H., Yu, R., Staniszewski, A., Planel, E., Herman. M., Ho, L., Kreber. R., Honig, L. S., et al. (2008). Retromer deficiency observed in Alzheimer's disease causes hippocampal dysfunction, neurodegeneration, and Abeta accumulation. Proc Natl Acad Sci USA 105, 7327-7332.
Ng, C. H., Mok, S. Z., Koh, C., Ouyang, X., Fivaz, M. L., Tan, E. K., Dawson, V. L., Dawson, T. M., Yu, F., and Lim, K. L. (2009). Parkin protects against LRRK2 G2019S mutant-induced dopaminergic neurodegeneration in Drosophila. J Neurosci 29, 11257-11262.
Piccoli, G., Condliffe. S. B., Bauer, M., Giesert. F., Boldt, K., De Astis, S., Meixner, A., Sarioglu, H., Vogt-Weisenhorn, D. M., Wurst, W., et al. (2011). LRRK2 controls synaptic vesicle storage and mobilization within the recycling pool. J Neurosci 31, 2225-2237.
Rhinn, H., Marchand-Leroux, C., Croci, N., Plotkine, M., Scherman, D., and Escriou, V. (2008). Housekeeping while brain's storming Validation of normalizing factors for gene expression studies in a murine model of traumatic brain injury. BMC Mol Biol 9, 62.
Rogaeva. E., Meng, Y., Lee, J. H., Gu, Y., Kawarai, T., Zou, F., Katayama, T., Baldwin, C. T., Cheng, R., Hasegawa, H., et al. (2007). The neuronal sortilin-related receptor SORL1 is genetically associated with Alzheimer disease. Nat Genet 39, 168-177.
Rojas, R., van Vlijmen, T., Mardones, G. A., Prabhu, Y., Rojas, A. L., Mohammed, S., Heck, A. J., Raposo, G., van der Sluijs, P., and Bonifacino, J. S. (2008). Regulation of retromer recruitment to endosomes by sequential action of Rab5 and Rab7. J Cell Biol 183, 513-526.
Sakaguchi-Nakashima, A., Meir, J. Y., Jin, Y., Matsumoto, K., and Hisamoto, N. (2007). LRK-1, a C. elegans PARK8-related kinase, regulates axonal-dendritic polarity of SV proteins. Current biology: CB 17, 592-598.
Seaman. C. F. S. a. M. N. J. (2009). The Role of Retromer in Neurodegenerative Disease. In Intracellular Traffic and Neurodegenerative Disorders, pp. 125-140.
Seaman, M. N. (2004). Cargo-selective endosomal sorting for retrieval to the Golgi requires retromer. J Cell Biol 165, 111-122.
Seaman, M. N., McCaffery, J. M., and Emr, S. D. (1998). A membrane coat complex essential for endosome-to-Golgi retrograde transport in yeast. J Cell Biol 142, 665-681.
Shimizu, F., Katagiri, T., Suzuki, M., Watanabe, T. K., Okuno, S., Kuga, Y., Nagata, M., Fujiwara, T., Nakamura, Y., and Takahashi, E. (1997). Cloning and chromosome assignment to 1q32 of a human cDNA (RAB7L1) encoding a small GTP-binding protein, a member of the RAS superfamily. Cytogenet Cell Genet 77, 261-263.
Simon-Sanchez, J., Schulte, C., Bras, J. M., Sharma, M., Gibbs, J. R., Berg, D., Paisan-Ruiz, C., Lichtner, P., Scholz, S. W., Hernandez, D. G., et al. (2009). Genome-wide association study reveals genetic risk underlying Parkinson's disease. Nat Genet 41, 1308-1312.
Spano, S., Liu, X., and Galan, J. E. (2011). Proteolytic targeting of Rab29 by an effector protein distinguishes the intracellular compartments of human-adapted and broad-host Salmonella. Proc Natl Acad Sci USA 108, 18418-18423.
St. George-Hyslop, P., Mobley, W. C., and Christen, Y. (2009). Intracellular traffic and neurodegenerative disorders (Berlin; London, Springer).
Stafa, K., Trancikova. A., Webber, P. J., Glauser, L., West, A. B., and Moore. D. J. (2012). GTPase activity and neuronal toxicity of Parkinson's disease-associated LRRK2 is regulated by ArfGAP1. PLoS Genet 8, e1002526.
Thayanidhi, N., Helm, J. R., Nyez, D. C., Bentley, M., Liang. Y., and Hay, J. C. (2010). Alpha-synuclein delays endoplasmic reticulum (ER)-to-Golgi transport in mammalian cells by antagonizing ER/Golgi SNAREs. Mol Biol Cell 21, 1850-1863.
Tong, Y., Pisani, A., Martella, G., Karouani, M., Yamaguchi, H., Pothos, E. N., and Shen, J. (2009). R1441C mutation in LRRK2 impairs dopaminergic neurotransmission in mice. Proc Natl Acad Sci USA 106, 14622-14627.
Vilarino-Guell, C., Wider, C., Ross, O. A., Dachsel. J. C., Kachergus, J. M., Lincoln, S. J., Soto-Ortolaza. A. I., Cobb, S. A., Wilhoite, G. J., Bacon, J. A., et al. (2011). VPS35 mutations in Parkinson disease. Am J Hum Genet 89, 162-167.
Wu, J. S., and Luo, L. (2006). A protocol for dissecting Drosophila melanogaster brains for live imaging or immunostaining. Nat Protoc 1, 2110-2115.
Xia, Z., Dudek, H., Miranti, C. K., and Greenberg, M. E. (1996). Calcium influx via the NMDA receptor induces immediate early gene transcription by a MAP kinase/ERK-dependent mechanism. J Neurosci 16, 5425-5436.
Zhang, J., Schulze. K. L., Hiesinger, P. R., Suyama, K., Wang, S., Fish, M., Acar, M., Hoskins, R. A., Bellen, H. J., and Scott, M. P. (2007). Thirty-one flavors of Drosophila rab proteins. Genetics 176, 1307-1322.
Zheng. B., Liao, Z., Locascio, J. J., Lesniak, K. A., Roderick, S. S., Watt, M. L., Eklund, A. C., Zhang-James, Y., Kim, P. D., Hauser. M. A., et al. (2010). PGC-1alpha, a potential therapeutic target for early intervention in Parkinson's disease. Sci Transl Med 2, 52ra73.
Zimprich, A., Benet-Pages, A., Struhal, W., Graf, E., Eck, S. H., Offman, M. N., Haubenberger, D., Spielberger, S., Schulte, E. C., Lichtner, P., et al. (2011). A mutation in VPS35, encoding a subunit of the retromer complex, causes late-onset Parkinson disease. Am J Hum Genet 89, 168-175.

### SUPPLEMENTARY REFERENCES

Alberts, R., Terpstra, P., Li, Y., Breitling, R., Nap, J. P., and Jansen, R. C. (2007). Sequence polymorphisms cause many false cis eQTLs. PLoS One 2, e622.
Chen, L., Page, G. P., Mehta, T., Feng, R., and Cui, X. (2009). Single nucleotide polymorphisms affect both cis- and trans-eQTLs. Genomics 93, 501-508.
Consortium, I. H. (2003). The International HapMap Project. Nature 426, 789-796.
Fung, H. C., Scholz, S., Matarin, M., Simon-Sanchez, J., Hernandez, D., Britton, A., Gibbs, J. R., Langefeld, C., Stiegert, M. L., Schymick, J., et al. (2006). Genome-wide genotyping in Parkinson's disease and neurologically normal controls: first stage analysis and public release of data. Lancet Neurol 5, 911-916.
Goecks, J., Nekrutenko, A., and Taylor, J. (2010). Galaxy: a comprehensive approach for supporting accessible, reproducible, and transparent computational research in the life sciences. Genome Biol 11, R86.
Hamza, T. H., Zabetian, C. P., Tenesa, A., Laederach, A., Montimurro, J., Yearout, D., Kay, D. M., Doheny, K. F., Paschall, J., Pugh, E., et al. (2010). Common genetic variation in the HLA region is associated with late-onset sporadic Parkinson's disease. Nat Genet 42, 781-785.
Johnson, A. D., Handsaker, R. E., Pulit, S. L., Nizzari, M. M., O'Donnell, C. J., and de Bakker, P. I. (2008). SNAP: a web-based tool for identification and annotation of proxy SNPs using HapMap. Bioinformatics 24, 2938-2939.
Langmead, B., Trapnell, C., Pop, M., and Salzberg, S. L. (2009). Ultrafast and memory efficient alignment of short DNA sequences to the human genome. Genome Biol 10, R25.
Latourelle, J. C., Hendricks. A. E., Pankratz, N., Wilk, J. B., Halter, C., Nichols, W. C., Gusella, J. F., Destefano, A. L., Myers, R. H., and Foroud, T. (2011). Genomewide linkage study of modifiers of LRRK2-related Parkinson's disease. Mov Disord 26, 2039-2044.
Lill, C. M., Roehr, J. T., McQueen, M. B., Kavvoura, F. K., Bagade, S., Schjeide, B. M., Schjeide, L. M., Meissner, E., Zauft, U., Allen, N. C., et al. (2012). Comprehensive Research Synopsis and Systematic Meta-Analyses in Parkinson's Disease Genetics: The PDGene Database. PLoS Genet 8, e1002548.
Liu, X., Cheng, R., Verbitsky, M., Kisselev. S., Browne, A., Mejia-Sanatana, H., Louis, E. D., Cote, L. J., Andrews, H., Waters, C., et al. (2011). Genome-wide association study identifies candidate genes for Parkinson's disease in an Ashkenazi Jewish population. BMC Med Genet 12, 104.
Mailman, M. D., Feolo, M., Jin, Y., Kimura, M., Tryka, K., Bagoutdinov, R., Hao, L., Kiang, A., Paschall, J., Phan, L., et al. (2007). The NCBI dbGaP database of genotypes and phenotypes. Nat Genet 39, 1181-1186.
Maraganore, D. M., de Andrade, M., Lesnick, T. G., Strain. K. J., Farrer, M. J., Rocca, W. A., Pant, P. V., Frazer, K. A., Cox, D. R., and Ballinger, D. G. (2005). High-resolution wholegenome association study of Parkinson disease. Am J Hum Genet 77, 685-693.
Marder, K., Levy, G., Louis, E. D., Mejia-Santana, H., Cote, L., Andrews, H., Harris, J., Waters, C., Ford. B., Frucht, S., et al. (2003). Familial aggregation of early- and late-onset Parkinson's disease. Ann Neurol 54, 507-513.
Myers, A. J., Gibbs, J. R., Webster, J. A., Rohrer, K., Zhao, A., Marlowe. L., Kaleem, M., Leung, D., Bryden, L., Nath. P., et al. (2007). A survey of genetic human cortical gene expression. Nat Genet 39, 1494-1499.
Purcell, S., Neale, B., Todd-Brown, K., Thomas, L., Ferreira, M. A., Bender, D., Mailer, J., Sklar, P., de Bakker, P. I., Daly, M. J., et al. (2007). PLINK: a tool set for whole-genome association and population-based linkage analyses. Am J Hum Genet 81, 559-575.

What is claimed is:

1-46. (canceled)

47. A method of treating Parkinson's Disease in a subject comprising administering an effective amount of a composition that stabilizes retromer complex to a subject in need thereof.

48. A method of treating Parkinson's Disease in a subject comprising administering an effective amount of a composition that suppresses retromer complex dysfunction to a subject in need thereof.

49. A method of treating Parkinson's Disease in a subject comprising administering an effective amount of a composition that induces retromer complex function to a subject in need thereof.

50. A method of treating Parkinson's Disease in a subject comprising administering an effective amount of a composition that stabilizes retromer complex component interactions.

51. A method of treating Parkinson's Disease in a subject comprising administering an effective amount of a composition that reduces retromer complex-associated sorting defects.

52. A method of treating Parkinson's Disease in a subject comprising administering an effective amount of a composition that inhibits reduction in retromer complex-component levels.

53. A method of treating Parkinson's Disease in a subject comprising administering an effective amount of a composition that increases retromer complex-component levels.

54. A method of treating Parkinson's Disease in a subject comprising administering an effective amount of a composition that increases retromer complex activity.

55. The method of claims 47-54, wherein retromer complex comprises VPS35, VPS29, VPS26 or a combination thereof.

56. The method of claims 47-54, wherein the composition comprises a nucleic acid encoding a protein comprising 90% identity to SEQ ID NO: 14 or a fragment thereof.

57. The method of claims 47-54, wherein the composition comprises a protein comprising 90% identity to SEQ ID NO: 14 or a fragment thereof.

58. The method of claims 47-54, wherein the composition further comprises a pharmaceutically acceptable excipient.

59. The method of claims 47-54, wherein the subject has one or more mutations in the polypeptide VPS35, VPS29, or VPS26.

60. The method of claims 47-54, wherein the subject has a genetic variant in the LRRK2 or RAB7L1 gene.

61. The method of claim 60, wherein the genetic variant in the LRRK2 gene encodes a LRRK2 protein comprising a G2019S mutation.

62. The method of claim 60, wherein the genetic variant in the RAB7L1 gene comprises a guanine (G) nucleotide at SNP rs1572931.

63. The method of claim 60, wherein the genetic variant in the LRRK2 gene comprises SNP rs11176052.

64. The method of claims 47-54, wherein the subject has sporadic non-familial Parkinson's Disease.


SEQUENCE LISTING (30 sequences)

SEQ ID NO: 1 - DNA, 9752 bp, Homo sapiens
ctgaaagaaaaagataatcatcagagaagtacggggatgacaaagaaagaacagcgtcatagaaggcataagggaaacaaatgtcaaggagtggtcaactatgtcaaaacgaataagaacagagaaaactggatccttaaagatgagtagcttgaactaacctcctgtacctgggtaacgaacattctgggcaaaatatattgtgaaccatgctgatgttctttcgccacaaaacataagtgatagcctctctatcaagagacctagatttcctagtgtctggtcctgggctgttttcccatggccccgctggttctcttcctctattcctagaaaagtatagaaaatggagctctctttgcttgccattgagctcatgtcctgccaacttctacccaagtgacaggtaggtcagtaaagttacaggctgagaaattaaaattatttgccaaacactagttttatgattatctattaacaacagccacaacaaatcaagaaggagtcccagtgtatttcaattagcttctagctcaagtgatacatgagtagtatggctagaataggcattgtagaagtatgaatggagaactgtctcttgttttctgtccatctttattgttattgctggatattaaatgctgtaatttttggcataggtttttttccaaagactggtctcttgggtcaagttttcctgaacttgcctggtattgggcaagttcacacagaaaatatgagtcatttcacaaagtcctgggcttgtctatctacttgaaggaataggaaatggacttcacttggagcatgatggatgaggcttgggcttttactaatgaaataagtttcaatcttaactcagttaaacctttaaagacaggagaaaactatctggggatggattcaggccagtgagatcacgttgcacaaatctccccttaagggatctttactgacctagaattgtttcaaaataagattccaaactgtcgagggagtttgaagccttgaaacttggtgtaggtttgcttaaaacaaaataacgaatatactacatttcccaggaggcttcgcggtgagcctccgcactcggctggttctctttaccgcgaggaaagctgggaaatgtagtgccacaggcaaccctgcacgtgacgcttgcggaggaaggggagagagaggcgcgcgggagggcgtctagggaatcgaggtgccggctgctccttcctcacaatttggtttgtgctgcaaggggagggtccccatcatctggccccagtggtgtaaggagctgactgggattcagtcactgacttggagccgctcgggggaagtcccggtgggtgaggttccgcggcgcctggtccagtttctcggcagtcaggccaggagggggtggggaaggtgcgaacagccgggggctcggagctcgcgccctgcgccccacccaggttgcggccgcccggggaggaagcgcgtataggttgagtgcaaagtttccttttttgctttctcgtcagagcagcccagacaggcggtgggtgggaatgcctcacttcagtttgaagagggtccggatccaaaggggttaaaacgagcgacccccgatccccgaccacacttcccgcctccctaaaacgcacaccccgctagccatgggcagccgcgaccacctgttcaaagtgctggtggtgggggacgccgcagtgggcaagacgtcgctggtgcagcgatattcccaggacagcttcagcaaacactacaagtccacggtgggaggtgagacgtctcgggggtggggagggggagggggaggaggaggccccgaccgcggactcgagctggggacccaagtggggtggggatttgctgtcggttgttgggtccttcttcgggtcaggaagtactgtctgggaacctagatggtgtaattgccgggtgtttggtttacacgcctctcttgagtttcccgttagattttcccaaactgagcccactgccaggagaaaggtaagagcaaaggcgtgaagatgagaaatagggaagggaggatgaggatgagcaattgctgtttattgcaggcgtactgtgaacttaagtctccagcctttcagctgtccggcaaatctctccaatcagcagaagcagctgagatcctctgtggggcctcctggttaggtgccggctggggctgtagtcctgcccctctgttgagggaggaaccgcaaggtgtttaacctggcttcagtgggagaacgcaggaagcggaactggtctgaaggactgtagctttgttgtggggctgtgggtgggatggaagacacaaaatccccgaacgagccccttggctacgcagtcagatgcccatgagggctgatgagctgtgtttttgtccgttgattgcttaaataaaggtaaccaaggaattcttccaagaatgtgaaatataccacttctctgacctatcttcccaccacttttttcctcaaactggagaaggtagggaggaaggggacaccgagttcattcatttctttcccgggtgtggcctttggactcaactattttttcagaggcagaaatgccttattgttaattagaaggtatttagctaggaagagctggtgatgggggagagtaacttaaaattttatttctctctgttaaaagaacttgtttcaaggggggaaaactcgtaccaatggaaaaattgaaaatgtgtgctcctgttcatatggaagtttaaaaagaaattaacaaacatttacattgcaattaaaacattctccttaagaacccccagtagggcccagtaccgtggttcatacttgcaatcccagcactttaggaggctgaggcgggaggattgcttgagcccaggagttcgagaccagccatgggaaacatggcaagacccccacctctacaaaaattaaagaagaaagtagccgtggtcccagctactcgggaaactgagacgagaggattgcttgaactggggaggtcgaggcttcagtgagccccgttcgcaccactcactgcactgtagcctgggcaacagagtgagaccctgtctcaaaaacaaaggaacaaaaaatttaagtctggcaaaattaaaggttcaaagtttcagacactctatcctgaccgctctgccctcccactcctaaataatataagggtccctatttcacggatttgggttaaaagtggcctgctattaatagttaatttttaacaattgctgtagttttcctttagcaaagaatataattggctaatttttttttcagtgtttagtctaaatttcccttagctttgattcctgtcacagatgtgaaagataatatttccaaacctcagaaattactctgtttctctccttagccaaaaggctttccccatggaatacttactcagccttaaggagtctcagcacctgcagacttggtggtagattccagcaggactcttcagcaacatccacacagtgtcttcttcttgggactcctctgggaccttttccatttattccattcttgcccaagataatacctgcaaccttatggtggatgtagagttccccagggtgtgttcttttcataatacaatttgattagaaaccaacagtgaaattgaatgtggaagatacgaaagcaaccacacttgtctcatttcccccttctcccccatcaccccttccctgagaaaaccccctaaatctcccagaaactatttaacctgagaatgagaattgttttcatagttactcccacccccagtcttggagacaaacagtagcaaataaatgagcttgacagtagagtaaaagctggaaacccctttggagagtgagtatgaatcatccatctcacactaaatacacgcatgtggactcatctcagtgaattgagggcttaaagttaaacatatgggattggagttgtgtgtccatagggtttcactgcctatttgatttgagtttatccctattaattttttacagtgaaattttattaaagtataatgtacatatattttcagtggattttgctctgaaggttctccagtggtctgactacgagatagtgcggcttcagctgtgggatattgcaggtaaagtgggagggctagataggaacaagggaagaaattttgatttaactagaaagctctgaggaaaaaatggggcagcaaagccagaaattgtttttctgaagctggaatgattgagctgctctgaggacacatgagctgaataggtgcagagcttttgtagatgttttgccctgaatagatctggagcagtctctatgtggatggaggggctggcaaggtggtggattgtccagactataggtggacactcccacagcaggggagggtgctttttctaggtttaaggctgaccttggtgaaaaacaagatccccagaaaatagatacagtaaaaatagtgaatccttactatgtgccaagcattcttctaagtattttaaattcactgactcattaatcctcacatcaaccctatcataaaagctactgcttttggagaattgcttgaacctgggaggtggaggttgcagtgagcctagatggcaccactgcactccagcctgggcaacaggagtgcaggaaactgtttcaaaaaaaaaaaaaaaaagcgactgcttttatcttcactttacaaatgagaaatctgaagcactgagagcttgatttgcccgaagtcaaatagtatgcagcagagcagggatttggactggagcaatctgggtgcggaatctgccttggaaataattgggcttcattatccctttatgggatagaaaaccaagagtcagagggggaaagtgagtggccccatctgacacatatgcttactgccctcttctacaaggaatggggagaggccggctcttgggtcaccttgagaactctcccctttctccccagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaaggtatgtggccaatctcagaaaatggtccctagcctctatctgtggttagaaaggaagtaaaatgctctctgattctgggcatccatttccccttcttaagttggcaaaatcatctctacctccttcaaaggatgttgagggtgagtgagactttctttcttttttttttttttgagacggagtctcgctctgttgctcaggctggagtgcagtggcgcgatctcggctcactgccagctctgcctcccaggttcacgccattctcttgcctcagcctcccgagtagctgggactacaggtgcccaccaccacacccagctaatttttttgtatttttagtagagactgggtttcattgtgttagccaggatggtctcgctctcctgacctcatgatctgcccgcctcagcctcccaaagtgctgggattacaggcgtgagccactgcgcccagccaagggtgagactttctttgggaagaatgttgattatagatttccgatgctggtgttcttatccattggtggaaatatgtttaaaatccacagtatctgagccatattctttcttagttgcaggaagtttatgatactgccttttattttttctctttgctagtgtgatctgtccccttgggcagtgagccgggaccagattgaccggttcagtaaagagaacggtttcacaggttggacagaaacatcagtcaaggagaacaaaaatattaatgaggctatgaggtaagtagctcatgctgataggcatgcagatgtatccatcttccactgtggccctgtggaccctcttttcttatttctgagccaacaatgacagactgagccattggaatgctagcccctaaaggtcagggatactgtctttcctgagataactggggacaaaggggcatttaaaccttctggctttaccaaatactctctgatgataagtcagactacatctgccatttctgcttttcctccaaaataagcttctcaggcttattttttgtcttttagagtcctcattgaaaagatgatgagaaattccacagaagatatcatgtctttgtccacccaaggggactacatcaatctacaaaccaagtcctccagctggtcctgctgctagtagtgtttggcttattttccatcccagttctgggaggtcttttaagtctcttccctttggttgcccacctgacaattttattaagtacatttgaattgtctcctgactactgtccagtaaggaggcccattgtcacttagaaaagacacctggaacccatgtgcatttctgcatctcctggattagcctttcacatgttgctggctcacattagtgccagttagtgccttcggtgtaagatcttctcatcagccctcaatttgtgatccggaattttatgagaaggattagaaatcagcacctgcgttttagagatcataattctcacctacttctgagcttatttttccatttgatattcattgatatcatgacttccaattgagaggaaaatgagatcaaatgtcatttcccaaatttcttgtaggccgttgtttcagattctttctgtcttggaatgtaaacatctgattctggaatgcagaaggagggggtctgggcatctgtggatttttggctactagaagtgtcccagaagtcactgtatttttgaaacttctaacgtcataattaagtttctcttgtcttggcatcaagaatagtcaagttttttggccgggcatggtggctcatgcctgtaatcccagcacttggggaggccaaggcaggcggatcacatgaggccaggaattcgagaccaacctggtcagcatggcaaaaccccgtctctactaaaagtacaaaaattagccaggcgtgatggcacgtgtctgtaatcccagctactctggagactgaggtgggagaatcgcttgagactgggaggcagaggttgcagtgaaccgagatcatgccaccgcacttcagcctgggtgacagagaaggactccgtctcaaaaaaaaaagaaaaaagaatagtcatttttaaactacctatctcatgcaatgaaagcattttcttccacaaagagcttaatcctcatgataggattgcctagtgtctcccatttgcaggtttctgggttgatgtcttaatgcataatactgcaagtgacatcagctggctgtgatgcttcgaaataggtctgctcctcacagctttgggaatctgaatggaagaagaaaagagagaagttaacaacctccactggggcaactttgtgaacacgtaggcacttagtcataggaaacatattatgtgcaggtcctagcctgggggaggaaagtagatagacagaaaatcattaggtaatttaagtactaaattgggcagggctttttagtatcaaatcactactagaccatttaatttgttaaattatctctaggatggtgatttataacctacccaaagttatcgatattcttactaaactctgaggcctgaagttctgtgatagaccttaaataagtgtcctaagtcagtggttcccaaatctggctggtcgggaatacctgggaagtttgttaaaattttttaaaaatgttttaagatttttgggtcctgagccagccgtggtggctcacacctgtaatcccagcactttgggaggctgaggcaggtggatcgcctgaggtcaggagttcaagatcaacctggccaacatactgaaaccccgtctctactaaaaataagaaaaattagctgggcgtggtggcgggcacctgtaatcccagctacttgggagactgaggcaggagaatcgcttgaacctgggagttagaggttgcagtgagctgagatcacaccattgcgcttcagcctgggcaacaagagtgaaactccatctccaaaaaaaaaaaacaaaaagaaaaagatttttgggtcccgacctcaaacctactgaatcagaatttctagggatgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatattctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaaaatcacaaataaatgagaactaagtcttgtattcatggtggttattggcctttaatgtgagtttgtcaaagtgctgttttatactgatagctcaagaggattgcggaaatggagactttattttttaaatacctttttctaaattttcaattcaaggggtacgtgtgcaagtttgttacatggatatattgtgtgatgctaaagcttgggtttctgcttagcctgtctcccaagtagtgaacatagcacccagtcggcactttttcaacgcttactccctctctccccacttttgggagtctccagtgtctgttgttcccatctttatgtgtgtgcccaatgtttacctcccacttacaagtgagaaatgtggttaaataccttcttttaaaatgaaggtaaatattattctgtttacctgttaacttactaataccaattgcttagatccatctgcctagtaccaattcagtacttgatggaacatgccaaatactacttggaaacgtgacctttttgaaaaactgaatgtgcttgcttttcccatttgctgactcaggatttacctgccttttcaagcattgttagaggctggccagccagccagcccttctggatattctctcccaactggtgaacattataggcctgaaactaattaaagaaggaactgaaatatacctaattcctcttcgtgcccctatccataagagaaaagcaattatgattaggaaaaataaaatatttacatgaatggaaatgcgatgctactaaaaataattaggtcttatcactcggatgctacctctttggtgcattcaaaagaggtagcgtccaaaattgtttggaacgtagatatcgtagcccctccctagaggaggtcctacatttgagacaagatgcctctccaaccactcaggtggcttttggtcaaaaaagaaaacagttcccccagagtgttctcagaaaccctaagggagggctgagaacacagctgtggggtagtgtctgccctgactttcttctttgaacagaagaaatttggtcacttagctgagtgaccaaataagtggagcagaggagattgtaggtcagggaaggccctttagcagtaaagaatggtctgctgctttatcactactattgcgccagaatta

SEQ ID NO: 2 - DNA, 3324 bp, Homo sapiens
agtgccacaggcaaccctgcacgtgacgcttgcggaggaaggggagagagaggcgcgcgggagggcgtctagggaatcgaggtgccggctgctccttcctcacaatttggtttgtgctgcaaggggagggtccccatcatctggccccagtggtgtaaggagctgactgggattcagtcactgacttggagccgctcgggggaagtcccgcccagacaggcggtgggtgggaatgcctcacttcagtttgaagagggtccggatccaaaggggttaaaacgagcgacccccgatccccgaccacacttcccgcctccctaaaacgcacaccccgctagccatgggcagccgcgaccacctgttcaaagtgctggtggtgggggacgccgcagtgggcaagacgtcgctggtgcagcgatattcccaggacagcttcagcaaacactacaagtccacggtgggagtggattttgctctgaaggttctccagtggtctgactacgagatagtgcggcttcagctgtgggatattgcagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaagtgtgatctgtccccttgggcagtgagccgggaccagattgaccggttcagtaaagagaacggtttcacaggttggacagaaacatcagtcaaggagaacaaaaatattaatgaggctatgagagtcctcattgaaaagatgatgagaaattccacagaagatatcatgtctttgtccacccaaggggactacatcaatctacaaaccaagtcctccagctggtcctgctgctagtagtgtttggcttattttccatcccagttctgggaggtcttttaagtctcttccctttggttgcccacctgacaattttattaagtacatttgaattgtctcctgactactgtccagtaaggaggcccattgtcacttagaaaagacacctggaacccatgtgcatttctgcatctcctggattagcctttcacatgttgctggctcacattagtgccagttagtgccttcggtgtaagatcttctcatcagccctcaatttgtgatccggaattttatgagaaggattagaaatcagcacctgcgttttagagatcataattctcacctacttctgagcttatttttccatttgatattcattgatatcatgacttccaattgagaggaaaatgagatcaaatgtcatttcccaaatttcttgtaggccgttgtttcagattctttctgtcttggaatgtaaacatctgattctggaatgcagaaggagggggtctgggcatctgtggatttttggctactagaagtgtcccagaagtcactgtatttttgaaacttctaacgtcataattaagtttctcttgtcttggcatcaagaatagtcaagttttttggccgggcatggtggctcatgcctgtaatcccagcacttggggaggccaaggcaggcggatcacatgaggccaggaattcgagaccaacctggtcagcatggcaaaaccccgtctctactaaaagtacaaaaattagccaggcgtgatggcacgtgtctgtaatcccagctactctggagactgaggtgggagaatcgcttgagactgggaggcagaggttgcagtgaaccgagatcatgccaccgcacttcagcctgggtgacagagaaggactccgtctcaaaaaaaaaagaaaaaagaatagtcatttttaaactacctatctcatgcaatgaaagcattttcttccacaaagagcttaatcctcatgataggattgcctagtgtctcccatttgcaggtttctgggttgatgtcttaatgcataatactgcaagtgacatcagctggctgtgatgcttcgaaataggtctgctcctcacagctttgggaatctgaatggaagaagaaaagagagaagttaacaacctccactggggcaactttgtgaacacgtaggcacttagtcataggaaacatattatgtgcaggtcctagcctgggggaggaaagtagatagacagaaaatcattaggtaatttaagtactaaattgggcagggctttttagtatcaaatcactactagaccatttaatttgttaaattatctctaggatggtgatttataacctacccaaagttatcgatattcttactaaactctgaggcctgaagttctgtgatagaccttaaataagtgtcctaagtcagtggttcccaaatctggctggtcgggaatacctgggaagtttgttaaaattttttaaaaatgttttaagatttttgggtcctgagccagccgtggtggctcacacctgtaatcccagcactttgggaggctgaggcaggtggatcgcctgaggtcaggagttcaagatcaacctggccaacatactgaaaccccgtctctactaaaaataagaaaaattagctgggcgtggtggcgggcacctgtaatcccagctacttgggagactgaggcaggagaatcgcttgaacctgggagttagaggttgcagtgagctgagatcacaccattgcgcttcagcctgggcaacaagagtgaaactccatctccaaaaaaaaaaaacaaaaagaaaaagatttttgggtcccgacctcaaacctactgaatcagaatttctagggatgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatattctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaaaatcacaaataaatgagaactaagtcttgtaaaaaaaaaaaaaaaaa

SEQ ID NO: 3 - DNA, 3223 bp, Homo sapiens
agtgccacaggcaaccctgcacgtgacgcttgcggaggaaggggagagagaggcgcgcgggagggcgtctagggaatcgaggtgccggctgctccttcctcacaatttgcccagacaggcggtgggtgggaatgcctcacttcagtttgaagagggtccggatccaaaggggttaaaacgagcgacccccgatccccgaccacacttcccgcctccctaaaacgcacaccccgctagccatgggcagccgcgaccacctgttcaaagtgctggtggtgggggacgccgcagtgggcaagacgtcgctggtgcagcgatattcccaggacagcttcagcaaacactacaagtccacggtgggagtggattttgctctgaaggttctccagtggtctgactacgagatagtgcggcttcagctgtgggatattgcagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaagtgtgatctgtccccttgggcagtgagccgggaccagattgaccggttcagtaaagagaacggtttcacaggttggacagaaacatcagtcaaggagaacaaaaatattaatgaggctatgagagtcctcattgaaaagatgatgagaaattccacagaagatatcatgtctttgtccacccaaggggactacatcaatctacaaaccaagtcctccagctggtcctgctgctagtagtgtttggcttattttccatcccagttctgggaggtcttttaagtctcttccctttggttgcccacctgacaattttattaagtacatttgaattgtctcctgactactgtccagtaaggaggcccattgtcacttagaaaagacacctggaacccatgtgcatttctgcatctcctggattagcctttcacatgttgctggctcacattagtgccagttagtgccttcggtgtaagatcttctcatcagccctcaatttgtgatccggaattttatgagaaggattagaaatcagcacctgcgttttagagatcataattctcacctacttctgagcttatttttccatttgatattcattgatatcatgacttccaattgagaggaaaatgagatcaaatgtcatttcccaaatttcttgtaggccgttgtttcagattctttctgtcttggaatgtaaacatctgattctggaatgcagaaggagggggtctgggcatctgtggatttttggctactagaagtgtcccagaagtcactgtatttttgaaacttctaacgtcataattaagtttctcttgtcttggcatcaagaatagtcaagttttttggccgggcatggtggctcatgcctgtaatcccagcacttggggaggccaaggcaggcggatcacatgaggccaggaattcgagaccaacctggtcagcatggcaaaaccccgtctctactaaaagtacaaaaattagccaggcgtgatggcacgtgtctgtaatcccagctactctggagactgaggtgggagaatcgcttgagactgggaggcagaggttgcagtgaaccgagatcatgccaccgcacttcagcctgggtgacagagaaggactccgtctcaaaaaaaaaagaaaaaagaatagtcatttttaaactacctatctcatgcaatgaaagcattttcttccacaaagagcttaatcctcatgataggattgcctagtgtctcccatttgcaggtttctgggttgatgtcttaatgcataatactgcaagtgacatcagctggctgtgatgcttcgaaataggtctgctcctcacagctttgggaatctgaatggaagaagaaaagagagaagttaacaacctccactggggcaactttgtgaacacgtaggcacttagtcataggaaacatattatgtgcaggtcctagcctgggggaggaaagtagatagacagaaaatcattaggtaatttaagtactaaattgggcagggctttttagtatcaaatcactactagaccatttaatttgttaaattatctctaggatggtgatttataacctacccaaagttatcgatattcttactaaactctgaggcctgaagttctgtgatagaccttaaataagtgtcctaagtcagtggttcccaaatctggctggtcgggaatacctgggaagtttgttaaaattttttaaaaatgttttaagatttttgggtcctgagccagccgtggtggctcacacctgtaatcccagcactttgggaggctgaggcaggtggatcgcctgaggtcaggagttcaagatcaacctggccaacatactgaaaccccgtctctactaaaaataagaaaaattagctgggcgtggtggcgggcacctgtaatcccagctacttgggagactgaggcaggagaatcgcttgaacctgggagttagaggttgcagtgagctgagatcacaccattgcgcttcagcctgggcaacaagagtgaaactccatctccaaaaaaaaaaaacaaaaagaaaaagatttttgggtcccgacctcaaacctactgaatcagaatttctagggatgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatattctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaaaatcacaaataaatgagaactaagtcttgtaaaaaaaaaaaaaaaaa

SEQ ID NO: 4 - DNA, 3438 bp, Homo sapiens
agtgccacaggcaaccctgcacgtgacgcttgcggaggaaggggagagagaggcgcgcgggagggcgtctagggaatcgaggtgccggctgctccttcctcacaatttggtttgtgctgcaaggggagggtccccatcatctggccccagtggtgtaaggagctgactgggattcagtcactgacttggagccgctcgggggaagtcccggtgggtgaggttccgcggcgcctggtccagtttctcggcagtcaggccaggagggggtggggaaggtgcgaacagccgggggctcggagctcgcgccctgcgccccacccaggttgcggccgcccggggaggaagcgcgtataggttgagtgcaaagtttccttttttgctttctcgtcagagcagcccagacaggcggtgggtgggaatgcctcacttcagtttgaagagggtccggatccaaaggggttaaaacgagcgacccccgatccccgaccacacttcccgcctccctaaaacgcacaccccgctagccatgggcagccgcgaccacctgttcaaagtgctggtggtgggggacgccgcagtgggcaagacgtcgctggtgcagcgatattcccaggacagcttcagcaaacactacaagtccacggtgggagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaagtgtgatctgtccccttgggcagtgagccgggaccagattgaccggttcagtaaagagaacggtttcacaggttggacagaaacatcagtcaaggagaacaaaaatattaatgaggctatgagagtcctcattgaaaagatgatgagaaattccacagaagatatcatgtctttgtccacccaaggggactacatcaatctacaaaccaagtcctccagctggtcctgctgctagtagtgtttggcttattttccatcccagttctgggaggtcttttaagtctcttccctttggttgcccacctgacaattttattaagtacatttgaattgtctcctgactactgtccagtaaggaggcccattgtcacttagaaaagacacctggaacccatgtgcatttctgcatctcctggattagcctttcacatgttgctggctcacattagtgccagttagtgccttcggtgtaagatcttctcatcagccctcaatttgtgatccggaattttatgagaaggattagaaatcagcacctgcgttttagagatcataattctcacctacttctgagcttatttttccatttgatattcattgatatcatgacttccaattgagaggaaaatgagatcaaatgtcatttcccaaatttcttgtaggccgttgtttcagattctttctgtcttggaatgtaaacatctgattctggaatgcagaaggagggggtctgggcatctgtggatttttggctactagaagtgtcccagaagtcactgtatttttgaaacttctaacgtcataattaagtttctcttgtcttggcatcaagaatagtcaagttttttggccgggcatggtggctcatgcctgtaatcccagcacttggggaggccaaggcaggcggatcacatgaggccaggaattcgagaccaacctggtcagcatggcaaaaccccgtctctactaaaagtacaaaaattagccaggcgtgatggcacgtgtctgtaatcccagctactctggagactgaggtgggagaatcgcttgagactgggaggcagaggttgcagtgaaccgagatcatgccaccgcacttcagcctgggtgacagagaaggactccgtctcaaaaaaaaaagaaaaaagaatagtcatttttaaactacctatctcatgcaatgaaagcattttcttccacaaagagcttaatcctcatgataggattgcctagtgtctcccatttgcaggtttctgggttgatgtcttaatgcataatactgcaagtgacatcagctggctgtgatgcttcgaaataggtctgctcctcacagctttgggaatctgaatggaagaagaaaagagagaagttaacaacctccactggggcaactttgtgaacacgtaggcacttagtcataggaaacatattatgtgcaggtcctagcctgggggaggaaagtagatagacagaaaatcattaggtaatttaagtactaaattgggcagggctttttagtatcaaatcactactagaccatttaatttgttaaattatctctaggatggtgatttataacctacccaaagttatcgatattcttactaaactctgaggcctgaagttctgtgatagaccttaaataagtgtcctaagtcagtggttcccaaatctggctggtcgggaatacctgggaagtttgttaaaattttttaaaaatgttttaagatttttgggtcctgagccagccgtggtggctcacacctgtaatcccagcactttgggaggctgaggcaggtggatcgcctgaggtcaggagttcaagatcaacctggccaacatactgaaaccccgtctctactaaaaataagaaaaattagctgggcgtggtggcgggcacctgtaatcccagctacttgggagactgaggcaggagaatcgcttgaacctgggagttagaggttgcagtgagctgagatcacaccattgcgcttcagcctgggcaacaagagtgaaactccatctccaaaaaaaaaaaacaaaaagaaaaagatttttgggtcccgacctcaaacctactgaatcagaatttctagggatgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatattctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaaaatcacaaataaatgagaactaagtcttgtaaaaaaaaaaaaaaaaa

SEQ ID NO: 5 - DNA, 3070 bp, Homo sapiens
agtgccacaggcaaccctgcacgtgacgcttgcggaggaaggggagagagaggcgcgcgggagggcgtctagggaatcgaggtgccggctgctccttcctcacaatttggtttgtgctgcaaggggagggtccccatcatctggccccagtggtgtaaggagctgactgggattcagtcactgacttggagccgctcgggggaagtcccgtggattttgctctgaaggttctccagtggtctgactacgagatagtgcggcttcagctgtgggatattgcagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaagtgtgatctgtccccttgggcagtgagccgggaccagattgaccggttcagtaaagagaacggtttcacaggttggacagaaacatcagtcaaggagaacaaaaatattaatgaggctatgagagtcctcattgaaaagatgatgagaaattccacagaagatatcatgtctttgtccacccaaggggactacatcaatctacaaaccaagtcctccagctggtcctgctgctagtagtgtttggcttattttccatcccagttctgggaggtcttttaagtctcttccctttggttgcccacctgacaattttattaagtacatttgaattgtctcctgactactgtccagtaaggaggcccattgtcacttagaaaagacacctggaacccatgtgcatttctgcatctcctggattagcctttcacatgttgctggctcacattagtgccagttagtgccttcggtgtaagatcttctcatcagccctcaatttgtgatccggaattttatgagaaggattagaaatcagcacctgcgttttagagatcataattctcacctacttctgagcttatttttccatttgatattcattgatatcatgacttccaattgagaggaaaatgagatcaaatgtcatttcccaaatttcttgtaggccgttgtttcagattctttctgtcttggaatgtaaacatctgattctggaatgcagaaggagggggtctgggcatctgtggatttttggctactagaagtgtcccagaagtcactgtatttttgaaacttctaacgtcataattaagtttctcttgtcttggcatcaagaatagtcaagttttttggccgggcatggtggctcatgcctgtaatcccagcacttggggaggccaaggcaggcggatcacatgaggccaggaattcgagaccaacctggtcagcatggcaaaaccccgtctctactaaaagtacaaaaattagccaggcgtgatggcacgtgtctgtaatcccagctactctggagactgaggtgggagaatcgcttgagactgggaggcagaggttgcagtgaaccgagatcatgccaccgcacttcagcctgggtgacagagaaggactccgtctcaaaaaaaaaagaaaaaagaatagtcatttttaaactacctatctcatgcaatgaaagcattttcttccacaaagagcttaatcctcatgataggattgcctagtgtctcccatttgcaggtttctgggttgatgtcttaatgcataatactgcaagtgacatcagctggctgtgatgcttcgaaataggtctgctcctcacagctttgggaatctgaatggaagaagaaaagagagaagttaacaacctccactggggcaactttgtgaacacgtaggcacttagtcataggaaacatattatgtgcaggtcctagcctgggggaggaaagtagatagacagaaaatcattaggtaatttaagtactaaattgggcagggctttttagtatcaaatcactactagaccatttaatttgttaaattatctctaggatggtgatttataacctacccaaagttatcgatattcttactaaactctgaggcctgaagttctgtgatagaccttaaataagtgtcctaagtcagtggttcccaaatctggctggtcgggaatacctgggaagtttgttaaaattttttaaaaatgttttaagatttttgggtcctgagccagccgtggtggctcacacctgtaatcccagcactttgggaggctgaggcaggtggatcgcctgaggtcaggagttcaagatcaacctggccaacatactgaaaccccgtctctactaaaaataagaaaaattagctgggcgtggtggcgggcacctgtaatcccagctacttgggagactgaggcaggagaatcgcttgaacctgggagttagaggttgcagtgagctgagatcacaccattgcgcttcagcctgggcaacaagagtgaaactccatctccaaaaaaaaaaaacaaaaagaaaaagatttttgggtcccgacctcaaacctactgaatcagaatttctagggatgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatattctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaaaatcacaaataaatgagaactaagtcttgtaaaaaaaaaaaaaaaaa

SEQ ID NO: 6 - protein, 203 aa, Homo sapiens
MGSRDHLFKVLVVGDAAVGKTSLVQRYSQDSFSKHYKSTVGVDFALKVLQWSDYEIVRLQLWDIAGQERFTSMTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 7 - protein, 179 aa, Homo sapiens
MGSRDHLFKVLVVGDAAVGKTSLVQRYSQDSFSKHYKSTVGGQERFTSMTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 8 - protein, 131 aa, Homo sapiens
MTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 9 - DNA, 144275 bp, Homo sapiens
gcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcggtaatcacttgaaaataaactgtgcttttatttttgcaaactttctccccctccttacatttgcaaattttgtcctcctccccttgaccctgctcaaacccggactcttaaggagccgcaaactcccatatcctttccttagggcagaaagcagctgagaatttcaggaaggtcttcacctttttgacttttctccccgtttcagactaaaaaggagagggggtgctgtggattgtgactttgcttcttttccccacccacttgttttccagcctccaagttatttcaaggcaaaaatatccatgtgcctctgttgatcgtcttggactcctatatgagagtcgcgagtgtgcagcaggtaaaggcattgttttcacttcaactcattctcccttctgtttggaaggagacgttttactggcaatgttaatatagccgagagttcttggttattcccaaaatttggcttgaggaacctctgactgtgattttaagatgggaatattgttaaatcattacgcaatgtaaacgggatgaagagccccagtatgtgttccctgagtgtctttaagaagtaactttataaaaccaacagtatggatggtggtagaaggaggataaaaatgggttcggttttagtctcgttattggcaagatgaattcattagtgtttagacttgactattccaagtatcttcccaatacagagcatgtcctagatgagaagattatgaatagtttggaaaaggggaataattaatagtgataaaatgcaactttgtcactagcaaactcttgtagagttcagcactttttaaaattcaaagatttctagcctttagttgtagtataccttgtagtatctaaagaaagtgatgtcttatgagaccctcatagtttgcaactgttgtcatataaaatgcatgtagaagtgaaacttttacaatctgtaccataggaaacccagaaatttgctatgtatcttggatttttttttaaagggggccttaaaaatggtaattaagaatgatttacagtcaaaacaaaattataggccaaggtgataacttccttcggagcacttagagatttggggaactgaaatcagttttgtcatctgcatgttaactcatgcagagaaagagaattggactttgaactccttggaggtgcagtcagaaagccaatgtttcttaatggttgagaggcttgacagacatgaggcatctcaatctttaaagtggtgtgggtctatctttatcttgatgtttatctctgtatctagctgtatctagtctgggtgaaccatctagcttctttgatatgaggacatttacatctggaagaaatattttaatttgttttcaactgtgaaatattttccatctgactattatagattttcacgctgctatcaaaccaaaccaagaaaagatggaggcataataaagatgctgttcttttaagactcaaagtcggaattttgcctgtggaatatgagtcactttttgggcactggcctattgtgcttcctgctctgcacccacgtcatcccttcttacttgtctctgctttggtgttcagaagtgcctgattctggccaccttcattccctagactctgtacttgatagagtcactcctgcttgatactgctcaggacagtcagatcctgggtaggcgttttggtctgcagggtctagataaggcagtgctatacttgacaacccaggggagcctggaacatacttcctaattcttaattttagaaattgcccaagcctgagcatacttgtccggagtagttatgagtgtcacttagtatttctgcctagagagtaccagaggcaaagtatgctggaaaataaggaagagtttttttaaaagtaattaattacttttttggatatatcatagttgtatatattttggggatacatatgctatttgatacatgtatacaatgtgtaatgttcaaatcagggtaactggaatatccgtcacctcgaacatttttctttgtgttggcaacgttgcaatttctttcttctagctattttaaaatatgcaatgaattattaaccataatttccctgctatactattaaatattaacttaattgcttgtatctaattatatttttgtacacattaaccaccttctctttatccctccccatcctttcatttccagtctctggtaaccaccattctactctcttcctccatgagatccaccttttccgctcctacatatgagtgagattatgcaatattttatttctgtacctggcttatttaatttaacctaatgacctccagtcccacccatgctgttgcaaatgacaggatttcattttttatgactgaataatattccattgtgtatgtataccacattttcttttatttttagttaagtaattaatttagagacagggtctcactctgttgcccagactggagagcagtggtgtgatcaaagctcactgcaggcctgcaatcctgggctgaagtagtcctcctgcctcagcctcccaggtagctaggactataagcatgtgccaccatgctcagctaatttttttttctttttttactttttgtagagatggagtcttgctatgttgcccaggttagtttcaaactcctgacctcaagtaatcctcctgcctcggcctccatattttctttgttaatctgttgatagacatataaggtgattctgtattttaactattgtgaacagggctgcaataaacatgggagttcagatatatctttgatatactgatgttctttttttggatatatacccagcgatgagattactggatcatatgaaaattctctttttagttttttaagatacctccatactgtgtttcatcatggctgtgctactttatattcccatcagcagtgtaccaccattcccctttttctgcatccttaccagcatttgttattttttgtctttttgataatagccattctgcctgtggtgagataatatctcattatggttttgatttgcctctccctaatgattagtgaagtttaggatttttttttcatgtacctgttagccatttgtctgtcttcttttgagaaatgtctatttggatcttttgtccatttaaaaataagacttttttttttttttttttttttgctaattgagtttttttagttccttatgtattctggttgttaatcttttgttgaatggatattttgcaaatattttctccctttctttatgttgtctcttcactttgttaattattttctttgttgtgcagaagctttttagcttgatataatcccatttgcctatttttgttgtaattgcctgtgcttttgaggtcctaccccaaaaatcattgcacagaccaatgtcctgtagcatttccccagtgttttcttctagtagttgcatattttcaggtctgagatgtaagtctttaatccattttgaaatgatttgtgtatatggtgaaagctgtggatctagtttccttcttttgcacagccaatatttgattctcactgaaatctcactgccctcctggaaatattaccggatgttttactgtcatagcgaagtgagagtaagctgctcactgaggatcaaagagcttgtgacagacctaagactcaagtcttctcacaccttcaaaatctctttccatcatacaatctactagctgctgaattcgcaagctttttgtgcaagctagtaaaaagcaaaatggtttgatacaaatactgtggccatgctaggtacaatgacatcaatttaaattatcattggtcttaacaaggggatgtagaaaggggtctcctactgacattttaatactcacttaaaagtagtatttttccttcagatttctttatattattagtataattactgtaagtatcctttactgctttatatgttgaattagctggaagtgccaaaagaaaaactcttaatgataaatttaaggtattaaggtaaatttctccttcatttaatttaatagtaattcttaattactatttaaaataaagattaaggtttgtttctagatgccatttaacatgatattccagactgccagttttattttcaaagtttgtttcatattttattaatgtttcttcataaatgacagtctttagaaaattgacggttaagctaggtgctttatatttttcttttcctgcctatcttttcactgtgctcctaaattttacatctctttattctcaagggttcaacctttgaagaaggggagcaaaataaatgaaagtggctaaaattttttctttaacccctagactctttcctgttgtgcattaattacatgcttgagtttttagaataattataataaagtaaaactaccaatttaattgtattgtaactgtgcaagatgggaaccttctctcttagagagataagcttttaattgaatagattaatggatcaattgttacctctgctttgctgccagagattctatttaatcacagaagttccatgtagtgctggagagctcagttgcctgaatctttttgcaaagcgtttactgatactgttgcttcaccaaccaaaacaaacaggttttttccttgagtcagctttgtaggtacagagatgagtttggcatcctatgtgacttttttttttttttttttttttttttttaccaccagaagctgttcagaatgttattttcttaaatagttcggaaaaaagtcttgatgtattctatgaaagcacaaaaatagtcagtttctatgacagctggattgtcaacgtctgttcagcttacgtggaggaggatgtcctacttgagtagtataggtagaaatagctatcagaaattgccgcctttgaaagcaatttgaaattatgtaaaaggaagtaatgacaaaataaagcaatttatgtttaatctggaaaagatccaaaagtaatattgtaaagagatcttgagtaatcatttttatcttcctaaaatagccgttgtttactcccgtaagcgagtaagaaacttgtgccattattccttattgggtgcatatagatttctcaccttgtcattcaactccttgcaatattcaactttacttatgcatccagccttatcccaaaatagcctcttccctgtagcagcttccttatcatgtagcagcctactctcctcaccgctcatccgttcttatacaatctggcttaagctctacctcttcatattatattcttctctgagtaatttcaactcacactgagtcttactttcagtatttctattatattggtatttattacacatcacacttagatacttttccattagtctctagagggtacatatatgcactttctctttttttttttttttttgaccatgtcaagtatagttctatagtataatagaatgaattggagatcctttacatttagagagggaggagtctacagtaggaagaataggttaattttcatctccggtttgaaatcaggactttcaatttttttttcagaggtaaagagcaacttagtcaagttggcatcttgtaaacagactgagtgaagatatacttaaaatgcatctataatttcatattttatttcgaaatgtgaaagagcctactaggggtgtctgtgatctctagaccttatcaattcattctagagaaatctggagggagccattgaggagttctacctcctgtctattttatagagctctttctctttttctcctatcagatgtagattcaattgctaaaaatgccacgtttcttgcctctattattctagcttcattacttggggagcagccattctgataacttacattttgctactaaaatctccaacttcacctaatccttcattataagccacttcattttttcctataattaaaattttaaatatgtggaggaaattctgtcaggtagatatgacttaaaacctactaagggccaggtgcagaggctcacgcctgtaatcccagcactttgagaggccaagtcaggaggattgcttgagcccaggagcttgagaccagcctgggcaacagagcgagacccctgtcttcacaaaaaaaaaaaaaaaaaaattagctgggtgtggtggcacatacctgtagtcccagctacttgggaggctgaagtgggtggatcacctaaggacaggagttctaggctgcagtgatctatgattgcaccattgcactccagcctgggtgacagagtgagaccctatctcaaaaacaaaaacaaaaccaaaaaaaaaaaaaaaacaagaaaaaaaaagtactaaggatctggtatagccattcttgcacttaataatcttggtacaacctctaaaactattttttatagtttattttattttgccttatttagacaattggcatgtctatgttcttcataatttagaaattatatgtatttatatatataaattatatatattatatattatgtataaattaaattatatatatatataatatatatatatttgaaatggattcttgctctgtcatccaggctagagtgcagtggcacaatcttggctcactgcaccctctgcctcctgggttcaagcgattctccttcctcagcctcccgagtagctgagattacaggcgcccatcaccatgcctggctaatttttgtatttttagtagagactgggtttcaccatcttggccacattggtctcaaactgccggctgaaatgttgtattttttgtatgtctttctggtatgatttttggagaaaggtgtatcctaagaatacggcttgcttttgtttctgggtaagcattttagggtatcattttgttgtataaccattgtttacaagtgagataagcatctattccactaagattgaagagattcatgtttgactgagtatgctctattaacattctttaaaacatgtgaatatatgtctttcttgttttcaggtgggttggtcacttctgtgcaaattaatagaagtctgtccaggtacaatgcaaagcttaatgggaccccaggatgttggaaatgattgggaagtccttggtgttcaccagtaagtatgatagatatgtaaaacaaatggccttgagtatttatttgtacacatgacaaccttcccttgatacactgtgtttgcaatccaaggctactcctgtggaattctttaaaatacagatatttttccttgagtcaatgatttacatttatagagagctttaaactcagaagtttgatttagaaagcaaacatttaaggtaacatgtcagaagttattattttaataatataatcatataattataaaactggttaagttgtagatttttgatgagtacttttgaattcaaaccatgaagagattttggcttttaataatagaatcgatacaaaccactagttcttaaaaaaatgggaactgagaaaagttagttctgtaagtagtaatttgaaagttgatgttctactgtctttaaatagtacatttatatatatattcctatatatacagtaagtttaaactatggctttcagaaagagttaagaaagaggaaattaactttcagcacatctgtagccaaatcttgatagtaattttaccagctatgtttttgcagtttgcagcataatggcttcttagatgagactacttccttagccatcattaattaagaaaatattttctcaagaagaatgtgtttccaggaaaatacattttggatagctttgtttcttgacagttaaaaaatatcttctaagctactgaggaggctgaggaaggagaatcacttgaacctgggaggcggaggttgcagtgagcggaggttgcagtgagccgagattgcaccactgcactccagcctgggtgacagagcgacactctgtctcaaaaacaaacaaacaaacaaaccttttgtcattaactttaaatcttttttatacctaatatgacttttctttatcacagaaaaggaaattgtgaatattttttggcttccaatggtatatggtttatgaaaatttaatttatgaaaaattttcaggtgtttgtattgctgatcagtgtcaagtagtgctataaatttagacaaattagagctatgtgtttgtccataagtgaacatgtctgtgcttatacattttcccctctttgacaaatgtgttgctcttcttgttttcagtacataaagggtgtgttttggaaagagcatatttacaattaattggagttctcgtcttcaatctaatctctgtaattctatgtatcagttctaaagtatacagcatttgatgaggaattactcaaaatataccagtaattaggaattgtaactttaaatgtcccttggtttgggtgataatttccaggaagtccaaagatgagccagtctataacctcagggagtgtttgggaaactcatctagtcatattcctgtacaaaccaactgttcaaattaaattacataaaagtttatgtaggaaatttcattcactcactcacttactcattcactcactttgttcatccagtcattcatcttctattcattgaatgtttttgaagcctgtcctctgggtcagaaaccatgcagttgtgaagaagatagacacactgctgtctccagtggagtgtattagatcactcccagcaaaaattgattgtaaaacagatttctcttttttcaaggccttttccctccaaagacttaccagtactgaagaaaaatttcttccgtggtaataaagtcaggaattgtgggaatggtatagggagaggtaggggcagggtgattaggaggaaggctggcagagaatcgaagactggcttcattcaggtcctccaattgccaaatggagattatgcaacgtttcttgaatacatacaaaactctagatgtggccagctcagtcttcttccaataatgtaaagccaaacaatgctttgcaggaatagactagagattatattttgggattaataacatagggattaaaatcttatcttgaactaactaaacattattgatatgctaaattcactttttttttttttttttttttttttttttagacagagtctttctctgttgccaggctggcgtgcagtggcgtgatctcggctcactgtaacctctgtgtcccgggttcaagtgattctcctgcctcagcctctcgagtagctgggactgcaggagtacgccaccacgcccagctaatttttgtatttttagtagagacagggtttcatcatgttggccaggatggtcttgatctattgacctcatgatccccccgcctcggcgtcccaaagtgctgagattacaggcgtgagcaacggccaccggcccactactttttaatatatcattaatttctcttttaaaaacagtagcaatcaataatttaaatattcaaatgaattcttaatttatatacacaaactaacatctttattatatctctatattttaatatatcaacatgtctaagataatttataaatttacatcatatataaaaatgggtttgctctcgatgtatataaggcttcatgatattttgaatatggagttgggtgaaaatagtgaatctgaatatttgaatttgaatatttattggaaaataagtagtgcttttaactttttaaatgagacacataatagtcccctgttgatttttttttatttttttaactttattaaagtatagttgacaattaaaaattgtttatatttaaggtatacaattgatgatttgacatgtgtacattgtgaattattcaccacaatcaagctaattaacattccctgttagtttttataagcctggttcaggtttgtagaaagaaacaaacacacatggccaggcacggtggctcacacctataatcccagactttgggaggccaaggcaggaggatcacttgaacccagaagtttagaccagcctaggcaaaatagcaaaccctgtctctccaaaaaagaatgaaaaaattagcctggtgtggtggcatgtacctgtatccctagcaactcaggaggctgaggcaggaggattgctcacttgagcccaggagtttgaggtttcagtgagctatgattgcaccattgcattccagcctgggtgacacagcaagaccctgtctctaaaagcaggcaacaaaaacacatgagcttcactacagggaattaaatacaatgagagtaataaaaaataggtgagcaaaaaaaatgcaaataagcaaacttttgagtatgatatttcattcttatcttgatttctgtttttaactccagattgattcttaaaatgctaacagttcataatgccagtgtaaacttgtcagtgattggactgaagaccttagatctcctcctaacttcaggtaatatgtgtatatgttttttgtgttgattcaaattaaaaaaaaagttgataccattaagtaaatgtgtgtgtgtgtgtttttttttttttttttttgaagatcaggattagggtagcttgatttaaatgtcctaaaattgcatctgtttttagacctagtgatgggacagccataatataatctaaatatcagttatttccaaaattctttctgtttccatctcttctccttatctcttttctctatactttgcctctcaaaaatctcattcaatacattggttttaaacattaccttatatattatccccaaatctctgttggtagtccgatgtttgctcccaaaatctggacctacatctcatattcccccaggttagtggtcattcctgcccttgccattattacttctttctccctatatatctttaataaattttctataatatatgttctggagtatgccataatcgttacattttgaaaacacatagtattacttcttgagtatttgctagatgccaggcttcacaagtaaaatgcttcacgtgcttttaaacacctgaatctgaaaacaccccttgagatagggattttatctcagttttctaagtgacaaacactgaagtgcagagaagttgctttggccactaagaggtagaaacagggtctgattctccatgtcaggttccttccctgagaaaactttggcctggtagataatggacctgaaaacaaaaaatcttgaaatgatgcaacagttgtgggcattgctgtgctggacactggctattatataaggttccgagaagaaaggccgctcacagggagctaatcttgaagggctgggaggagtttccttccatgtaggggagggctttttaggttgagagaagtatgggtgcagaggcctgggaggatagcatgagagaggctggacgtgtgattgggaaggtttgaattgtcctcatcagtcctgctaagagatgtaaagaccatgctggagaaagaggaagatgagagtatgagggaaaaacaagaaggtactcaacatttcactacagctttttatgaccatgttgtatggcatgcactaagagtctttaaccatgatttaatttaacctcacccttgggaggtattttttttttgacggagattcattctccatttggcgatggacaggaagatgagggtttattaatatgaaaaatctaccaacactggaatatattgaagttagcctcatacagtactactactcctattccagtattattatttttattgacagaatagatgctgtttgtgttaagttttggattatgataggaaatgtttggtatagtaaaaggcaagagtgtgacatgcagttagtccaagtacgaagagataccaaaaaaaaaatgtttagtgaggagcagagtttagcatatttggagtgaagacaatgtgcggaaggaaaggagctgatgagatacatgtactatagtcggttgtgtgaaaggtcttgtttttcatactaaggatcatgagaagatcttagtagattccagcaagggatttgcaagaccacatttgtgttttagaaatataatgcaggcaataaaccagttggatagaaattggggactgtagagcaattaagcaactgtttttgcattctagatgagaatgcaaaacacaataggaatgaaggtgccttgttcaaaaggagttttgttcaaaaggaatcttcaagatgtgtaggagatattcttaaaggacttggtaatgaattgatttgttgcttggatagagaatgagaagagaaaggagggtggaagaaggaagatgacttaggagtttctcttgggtagctagtggattatggtatcattgatgaagacagggaacaggagtaggccaggtttggggtaactgtggaatattcagatgttgtctaagaggcataagaatgtatttcagatgtttggggcaagttgtctaggctagaagtactgattgagactcatgaaattatagtaaagtgaactgggagtttatctcatttataaagatctagagcttgataagtctaacatctagggcagttaagtagtttatcaacaaacaaacaaacaaacaaacaagaaaaccatgggtctacaaaccattcacagtcttcatgtaaaaattaattcatgtaaaaattaacacattaaatgttaaagcagctctttactcagagcatattattctctttaaaataggtaaaatcaccttgctgatattggatgaagaaagtgatattttcatgttaatttttgatgccatgcactcatttccagccaatgatgaagtccagaaacttggatgcaaagctttacatgtgctgtttgagagaggtattttaaaatgtcaaattccttaaagtatatataagaaaaaaaggcttatactgggaaaagtagaacacagttataataagaagaaggtttctaaaatcctactatttattaagaagtgggagttgtctgtcaagggtgaggaatggggttaattcagaagtattgcttgttttggtggggtgaatttcattcgtgggttataaatcatgcccctggagtagactttcttcaattgcttaacaaggcataaggtttactttgaaaactggatgtgtgggtgctatgaaagaaaaaataaaactgtgaagccaagcataggttacactgggattatgatgttgagtcatcaccagaaatcatagaaattgcataaagagcctgaaggtttacaaagtgtccttcaggaaaaagactaatatgcatttcatagcctggccctgagattgataactgagattattatgtaattttagagttggttggagtccttgtttagtctttccattgaccttaggaggaagtgggtcacagcagtgaagtgagcatcctgcctgaggacacagagcttgtgacagtacagttcaattagcaattattttaagagccccttttgtatcattatgagagccaactgtgctaggggtttagataagaatgatttatgtgggccctgtgtcagttatcagtttaccagtctaatttcttgcagttcccagaatgggatagatcacctgataactgttgaattccctgtctcctcccagaaggattttaaacagcttatagataattataatacacaagagtaaacaaaatggatgagaaaataggtgaagggacaataatataaagctagattaagtttactgtgtttctaaggtcctgcatatttacaaaggggtgggccagaaatttgtctgtttgcttcctatctgacaaagaaaagaggtaaatatcagtggttacaaagttccttaagataaaagtaaacctattattcaggagaagcaattggtctcatgggagatctgagaaacatcttcccatgggttttcctggatgagacaataaaggacatacattttgcaaggaatacaaagtgtattgcagcgagagtgactctgtcaaaagtcagaatagcatgggcctggtacccagctctttgataatcatacaccgtgaagtagaagatagtttacagcgagtacggaattccttcaggctgtcatgtataaatgttctatcttgcaactaagctttcgatgacaattaggataaagtttgaggttctattgtcttgcagggtctgtaatcttctgtgtggaaggttaggggcacattcttcttcctggaaggagggctagcatcactttatcaccatcgttgtttagtccatctaagacactggaggtagaccatagaatgttacaaagaagaatgttgctcaatagaaaaaccatcagtgctgagagggttatgactataaatgtagagtagaaaaatttctgatttttccaggagtatcaggttctccaggactcaggggtgactataaagttaattttcaaaatttgaaagtgtactgtggaaactagaccataaagtgagaaagttccatgatattcttcacttgttaggaaaacttaactgatttcacattatattatagggacactctggcataaaattaaaaaaaatgaatgttgatcacttagagtgctgtgttttctaacatatttctggcgccattctcaagctagataaactataattttatacatgtttttcaggttgttgcccaataacaatgactccaaatggaacttactggcttgatcaaatgactttaattgtgaaaattaatgatttatatttttgctgtctgatggaaaaccactaagacagagtatttcaaagtctgattacttgccatttgctcaagttgacaactcttgaactgaaacatttagccgagctgcccttcagcagcctaccattaatgcctcccttttaaatattgcaatatgtccagttccagttggccatctttattagtcactgtcagttttctctagaatttcccaaatgaaattgtaaataattttgtttttctgagaactgcttgctgactagcacttttacatttcaaaacatggagtacctaacataggccgaaacaaaattatttgaatctccgtagcttgttttctcattataacattcttaggaagggctgcttcacagaaatatattttttatttaaggagattacacttgatgtatctcacacaactataatgaatattgtaatttttgaataattaaactttcatatcatctttaagcttattcagtattttgtctttcatttttaagtctcagaggagcaactgactgaatttgttgagaacaaagattatatgatattgttaagtgcgttaacaaattttaaagatgaagaggaaattgtgcttcatgtgctgcattgtttacattccctagcgattccttgtaagtagcatttaaatgttatttattttttgtatctgaaaaattacaatatatctcattctgagtatattttaacaatatttttattatttagaaacttgtggatgctcaacccattcattcatttattcatttaattaatttacattcactgacattatactgaagttggctgtgggcttggtgctggagaaacaatcgtggaaaatacagatgtgttccttaccttttcagagcttgtagtacaatgggggacacagataagtacagaggtgattacagtggcagaagtgatggcagatggcagaagtacctagagttaggagatcaaataggaagtgaggcagtgtctcttagcaaagatttaataagtggagcttcctgtgcatgaaggtgtgacctgaagtgagaatgcaggcaaagtggcccaggcagtgggcatggttaatgtaaagatgctggagcaagagagagcagactgccttcaagaagacaaaagtagctcagtaaaggtgtggggttatgagtgtgcgtgcatgcatgcgtgtgtgccgctgtgcatgcacatccccaaatatcctatccgtttgtgtttcattgacagaggcaagggagagcttgataagaggcagtaaatgaggccagagacatggagtggagagcatgaagggcctaaaaagccacatgaaggagtttgaattttattgtgactcttgattagcattttaatgaggctttgaaatttagccacatttttcaccaaaaatattaatcagaagaaattaatttgatgtgtatgctaccaatgattgctattaggctaaaataatggttcatattctgttttgttttgtattaatggttcatattctgttttgttttgtaagtgaccattaacactttgtattttatgtattacttgtgtgggtttctacaggatatacatatgcatttatctagtgatattttcatcctcacacatgtgaagttttgaggattagagttaaacaatgtacctggtatgtaataagtgttctaaaatcactgacaggattattagacaatatgtattttatatgtgtgttgtatactatatgtaattgcatttatggtttcagatatggaaatcactgtgtcaatctgaaggtgtgagccttcggtgtaggcagagtaaaacccaatgcccttgtgaaagaatgcttttttttggtgatgtttataaaatcacaatgttttcttatccacaggaaattaaacactggaaagtgggtggggctgaacaataatagagaaaggccatggttttacatttctctgagacatcactgccaacaaactgaatatgtttttcattatactttttccttggctatatttattcatttatttatttattttgggctggaggtttttggaatccattgttttccacccacattggacataactccagtaaaaatgtgttgattcataatgcaaaagtcaagaaagtagcagctaaaaattaagaaatcaaaagtttttaaaacactgattctaactgaaaaacatttgcttttcagtctttaagtctattgttctgagtcaaagcagttcatttccttacgttgttaattttttttctatgtttaagcattgtaatatactttttgtgaaaacagttgattagttttggttgtgccaaaacaaatactaaaatgttttgcaaacagcctttttttaaacaaaaaaagaacagttaacatttgatgcagagatatacatgttttctccatgtaggttcacacctcacttcctttattgattaattgctttttctggtagagtctttctttcctttctgttttacctgtgtttgtccctaagacttatattttaatattatgcctcctctctttcgttctcccatcttttcttccacctattttggagccttcaggaagcttgattttgctgccttgtacattggttgcccttctggaatggaggaaacaggtcatagctgattttaactgttccatctggtgacatattcttgattttctttcttttggttggggaaaaaaaaacaatgcaaaagtcattctccaatggggttgagcctcgttaagaaatagaccctccacaatggttgaactagtttacagtcccaccaacagtgtaaaagtgttcctatttctccacatcaaaaaaaaaaaaaggtaagcaatataacatgagccatatctaataggacttcagaaattatctatcctatagttccaggatgacgatgatgatggttgtgataatgatgaagattgtgatgatagttatatgggagataaaactttaagcactttacatattaaattctataatattcaccacatctattaaaatatgttacattattgtccctattttaccaacaagaaaactgaccaacaagattaaacaacgtgactaagttcacacaacctgtaacagcagaatctatgtcaatcacaacacaattagcagctatttctgtggcaattttcaataaagatgtgtctggaaaaaaaaaaaagaaatagaccctccaatttatttatctgaaaacttatgaccaatacattacatttccagactttcattttcagtacttttcctttcattttcagtactaaaagtactctgaatttttcctttttttgatcttaaggctttaagccaagaaacaggaataaagtaaattttccttaatgccaaagattagtcctacaccccattatgttattaatgaacagcatagtattttttacagctacttaaagaacatgatgtttaaatttggaaatgcagtcattatgctgccatctatttacagtctatataagacgtctttgtatgcatatttgaaaggagaacatggttaccttattgataattatgatctctttaaattcaggcaataatgtggaagtcctcatgagtggcaatgtcaggtgttataatattgtggtggaagctatgaaagcattccctatgagtgaaagaattcaagaagtgagttgctgtttgctccataggcttacattaggtgagtttcttagttaatatgtcatcacacactgtatgatatacatatacatataaaacatatatatgttgcataataatggataagtagcatattgacatactttgaatgaaaatattgtaaaatcccagaaaaaataaattaaaacaaaaagaaaatactgtaaattaccaaactgttctgctgtgcttagatggacttttaaaaggagtgtcaaaaatagatgtgtagaatgtaaaagaagtattatcttaatcttatttttatagatgtagttctatagatgagtttttttattgttgaggctatatttaaaatataattatgtaagaattgatacatacaaaaatatgcataacatatacaatataaagcataatgcaaataactcactatccaacttaaatgttgtatattcccagtgggggaagctaccctgggcttcttcctgcccttgctttctctatagaggttaacactatccagaattttgtgtttacaaatcttttgtttataaatatgatttactacattttcatgtttctctaagcaaaattgttaatgtttgcctgcttttgcttcatcaaaatgtaatcatactgtatgttgtcttctgcaattttcaaatatcagtgctatgattataagaatcagaaatatttttgcatgtggttgcatgtagtattctattatttggaaataccacaatttatttttccatttttctatccatggacatttggattctttcttattttatgctactactatctgtgttaaaacagataactgaaaaagaacagttaacatttggtgcagagatatacatattttctccatatataaataagggttaatattttaaaaaatatttatttgccattggtatgtccttctttgtgaaatatgcaatctgtcatttatcaatattgttcacaggatagtctgtctttttaaaaccgattcatagattctggataataatgttttggtggtaggttatacgtattgaaaataccttcccttgaacatcacactctggggactgttgtggggtggggggaggggggagggatagcattgggagatatacctaatgctagatgacgagttagtgggtgcagcgcaccaacatggcacatgtatacatatgtaactaacctgaacattgtacacatgtaccctaaaacttaaagtataataataattttaaaaaaaaggagatgaagaggtagctgcaggttgactgagcaagggtcattgtctatttgaagtttcaaaggtatctctgaaaataaacacagttttttgcaagagtgaaaaaaaaaaaaaaaagaaaataccttcccttgacttgtagcatgtcttttcaccgtctttatggtggcttttgtgatatagttaaatttaataatcagttcctttgtgatttactcttttttatgtatcttgtttaagaaatcttaatctgtcctttccctaagataataaatatattgtatattttattccaaatcttactcgtttgttaaactgttgcagtttgtttttgtaaagaacccaattccccctctttttttcagtgtggagagctagttaccatcacagcacaatttattagaggttcacctgtttcccagtcatgtggtatataaatatgtaaacatatatgcttatgtttctgtccttggcgttctgttgcattaatctgatttgtctgaatcagatttaaatctgatgttaaatataacactatttaaattaatagttctgtaagtcctgatatctgaaaagctaataggtcaagtcaccatacattcttttttaggcacagcttgccttttttcttgggcctttactattccatgcaaattacaggattaacttgtccagttccttggaaaacactgttgagattttgactggaattgcacaaaatatgtggatcattttaagaagaactgacatctttacattataatttcttctatacatgtcttgcacatcttagtttttgctacgtatcttattttttattattattgtaaatggtatttctttttaaatcatatttttgaactgtgttttcataaagaaatgcaaatgattttttgtatattgaactaacctacctttctaaactcttcgttaattctgacaatttgtttctaaatgcacttaagttctctacctagcaattatataatttgtaaataatgacagtctgagttcttcctttctgcttcttatattctttatttcatttccttgtcttcttacatgagtcagaattattaatatagtgttaaatagagccattgatactagacatccttgtcttgctctttttcctgattttaaacagaatatttttaatattctcccatccaaaataatgcatcttacaagggtttaaacttttaaaaaaaattttgttaagaaattttattttatttcttctttgcttttgttttttcaatgtgggcttttaaatgtttgcttttatttttacaatgtgggcttaaaaatattaaaatatttaattttatcaaatatacttaaaatgtagtaagtcttttttttccttttcattatgttaatatgaagaaatacatctacaggttttctaatactaagctactgtgcacttcctgataaatttaacttggtcatttattagatttttaaaaacacttctaaaaaatcttctctgtttattttcacatataccaggtgagttttggcagcctcttgttttttcttttttaccctttcttttttgttaaaaacatccttttatttatttaatgattaatacatggctattttgtgttttgtatctgataactatatataaagttctggggagtctaaaccaactgcttctaggctgactgcctgtcagttgcggagccttgttttcttgtatggttgtgagtttctcttttattgagcatcctgaggacttaaattaaagatgctctttcagaggtgtttgccaggagtcagagcacaggactgacctgggagcagtttaggatatgatccaggcttaatatgggagactctggttgagaccttaccttgcagagagtctgaaactggtttgttgaatgcagcgccaggattcatgctttcccacaagactactctggcgttcaactcacagctcttgtttcagcttctttttgaactccctctgcccctcaacacacaccttggaaatttccttgatatttttgtgaggacaacaatgcatttaaaagtgagagtggttgctgaataataaggaatgatcgttaactgttggacattgattctgatgacattttctcaaaaggataaccaggaatattttgtacaaaattaggattattataataggcatttagtttttcattgttacaaattttgaggaaaatcattaatcatttgaaaaaactaattgacatgtctccattgtagcaacttgtattttcacttctagtatcatgattatatcccctgtgtaatttggaaattgattttagcattaggaaatttcctagtttcagttaaaatgaattttttgtaagctgaattctattttacatgcacaactttagtttgttatttctttccttgacaagcatttattgaatgttgttatatgactaaaactgtaagatgataatgttttaatattttcacatttctttcatagcttccaaagtgtatatatatactcacatacttcatatatatgaatctacttatatactgtatataaaaatatgtatataaatatatacacattgtatataaatgtgtatatatatttacacatgtatataaatatacatacaaatgtatataaatatacatatttatacacagatgtatataaatatacatatttatacacagatgtatataaatatacatatttatacacagatgtatataaatatacatatttatacacagatgtatataaatatacaaatataaatatatacatttatatataaatatacatatttatacacagatgtatataaatatacaaatataaatatatacatttatatataaatatacatatttatacacagatgtatataaatatacaaatataaatatatacatttatatataaatatatatattttttgatacgtacaactatcttgggagatgggatattgttattatttccacatttacagatgaggtcctgtggttcatcaatctttgtgttttattcaagattatgtaagcagtaagggatgaaatcaggctgagaacccaaatttcttcatccctaaacaaagtatttcttctaaacatggtatccatttactagtttatctctatcaggtgaccctttattcattatttttcatgagaaggttgtagttgtacaaagtggctgatatctgataatgttttaatctaattcaaagtcgatttcttaaatccaggtgtcagagtagcacactactgtacaattctctgtttcatgttttacaatttaccacagtcaagttacaacttgcacatgttacattaaaatgtgaattcaccttaatttcttgaaatgagccagaagaagaagtggttttgttttgtgatcagggaaatgctacttgactgccaaattgtctagaacagcacattaaagttgcttgattttatactgttaaaattaaataaaacatggcaactgtcatgtcatatgtaacttttgtattattctgtaactttttttgaaaataaaaagtgatcaaattgatcttcaggtaaagaatcttttttctcttgattatctcttagtggatgatgatttgttccatttaatgggtagagaatttattgttgctgttattgtttcaaaagtagctgaatgaaactcttaactttttcttcatagttaaaattaaaacctcaagtaaataaatatttaacgttttgccaaactatgtaatctaattatgggcttaatgcatttaaaggctttgtataatttgctacgtattttcacacaagttacctgaacataagtccagtcttcctgctgttttctgagtcacacagtgattcagtgaccgaacagctgttggcagtggtgtgggtataatagggaaaagagactgatggggacaacccaagtttagacaagctggtaaaagtagaagaaaatcttcttgaaaacactagtgatcactaagggctgtggagaatttttgcttggtgggtgaatgtggaagaggcaacagcatgggaaggtgttggtaaaggagctccatacttgcttaaactgccttttgattgtgaggccgttgatgaatatttagtttgggctttaggttttttatgatacaggatattttccatttctggctttgtatctcagagatcacttagttacacttatagatgaataggagtttcaattccttgttttagaaagaagcttggtaactgttagtgagttacaaataagccaaatagaagaaggtacatatttctgcagtatcaggtaaagtttttcctcataaggatttagactcttggatatcatattaattctcagaagagtgggtataaaaaggtatgggacttcttcctggggtgggttggaggtggtgaaatacctttttttttttttttctgagatcatcatagacaagatcaaataatggtaaacatgccaatgaattttctaagcactattcctttaagtgaaagaagagtgtttcagtaaaatgatttaatattgggtcttccaaaagatggatttaagagtttcaactttaaaagacagaaaaattaagttattttacacaatgaatattgtcgtgccgtgtgtcacagacatgacatgagagggaatcagagaacatacagttaatacaacgcaaactagtatcattacttttgctcaatcacttccattgtctaagtaagataattaaaggacagcataaaataaaatttcaaaactttactcaatcatattaagctattttaattaaagtaaatgttttaatgccattgaatattcatcaccattcaaaattattgatgtaaatagtgttatatgttaaaggtaatttaacttccatggatgagaattcagctaatgtttcacttaacttttaggtaattttttcaatatcctggtattaaacgaagtccatgagtttgtggtgaaagctgtgcagcagtacccagagaatgcagcattgcagatctcagcgctcagctgtttggccctcctcagtaagtaacttcactaaaaaggggattcttacagaggcatttgacatcaaatatgaacattgtaacaagagaatcatatgtacagatggaagcattcaatgccttttctgtcctgtgtagctcattttccagtagaggatactttcaaggaaactaacagttgtgacaaatatacacatctcaatgtagagttttgctttacatcattcttgatttagctttgtcattaagcagctaatctgttttaaaaaaatttttatttgtgcctgggcatggtggctcacgcctgtaatctcagaactttgggaggccgaggcgggtggatcacaaggtcaggagttcgagaccagcctggccaacatggtgaaaccccatctctactaaaaatataaaaattagtctggcatggtggcgggcacctataatcccagctgctcgggaggccgaggcaggagtatcgcttggaactggagggtaggagttgaagtgagctgagattgtgccactgcactccagcctgggcaacaagaatgaaactccatctcaaaaaaaataatttatttgtgttttaagttctggtgtacacgtgcaggatgtgcaggtttttcacataggtagacgtgtgccatggtggtttgctatacctattaacccatcacctaggtattaagcccagcatgcattagctatttttcctaatgctctcccttctccagtcccatcccctaacaggccccagtgtgtattgtccctctccctgtgttcatgtgttctcattgttcagctcccatttataagtgagaacacgcagtgtgtggttttctcttcatgtgttagttatctgaggataatggtttccagctccatccgtgtccctgcaaaggacatgagataattcctttttatggctgcatagtattccatggtatatatgtaccacattttctttatctagtctatcattgatgggcatttgggttgattccatgtctttgctcttgtgaatagtgctgtgatgaactaatcttttcaaataatcctcctctcgtctattaggttttttttttttttggtaccttcttcctcattttattatttatctggataggatggtagcattatgagacgtataatatattaaaaattatctttataattgaccaaggcttctcctaaagcacacctcattctgttggtaatatttcaaaatatggacttagagttggtcaaactgttaagtagataatatatataatgtttttatatatttttcaatttttttcaagctgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggtaggactctcataaatattagagttattcaaaattatgttttccagtcatttatattttgacagatttcttttttctcccctaatccaggaggccgcatgctgggcactaaataatctccttatgtaccaaaacagtttacatgagaagattggagatgaagatggccagttagtagttttgattttatatgatagaaaatttcagttatattttaaatcaatacctataaaataccttaaccgtaacttttattgttagaaatatttttgatataggcatttagttttagatgttgctgcaaaatagtagtaggtatgtagtattttgatctcatcaccttcaggagttagaaaaggtagaatgagagttattattgagagatttggaatcaagggtcatttggtaattcatgaatcatggagaaagaatctttctattttctggctgatcgttttaaaatgccatattaattcatcttgggtgatagaaattgcagagccatctgtgatcttttcttctatggtgactagccagcaggttgtcaatatcagaaattagatttggttagagagcttcctatgatgagttcccaactgatgtgacagagttgacctgtcttctttcgagagggttatttgaagctgtcatctctggataactctttcaataggagtgccattcaaacatcataagaccggcactctctcccaaagatacaagctgtagcaaggagttttgtgcatatcaggtttgtttcatatccgtgagcctttgtgttttatggcaactgattgattatacttgtgctatttgcaatgggtatcctctgggttttaaatagtgaatgacttattttggaaacaaatagtagtattctatgtctgaaatcttgaccgtctatttgtttaattatctattgctgataagaagggaattaataacacagatgctacttaattaaatattttcattttgacaagaaacagtaattcttttgaaaactatgctaaattggcatcttaataatctcatgttgagcaaggcttttggagattagggtaagggagattcatgtcgcagtttataaatttcagttcataggatacctatttttcaatatccataagataactttaaaataaatatattattaaaacaaagaaaatatatttacgttatacatctttaaaacctaccttgcttctttacaattttcagtttttcctctcttgtttccattctcttctcctcactttacctttttcctcagcatctctttacatatctgctgagcttttttatttctctctgctgcatatgcttttgaaaaagcataaaaatactaatttgttaggatttaattaattcgagtcttaaaaaatgaactataattcactcttgtaagtggaggtggcatgaaatattgtttatatgctcttaattgttgttagagatatttgataatggcaagtgagaatttgagatagttatttaaaagattactactaacattttgtttgaatttttgaaagtttcccagctcatagggaagtgatgctctccatgctgatgcattcttcatcaaaggaagttttccaggcatctgcgaatgcattgtcaactctcttagaacaaaatggtaagcagtgggccatgttttcaaataaagggaaacacatttttgtggtatttttaattatagaagctatatactgtgaaaaatttacataatttataaagctatatattgtgaaagatatctctatgtgtagagatgtattgacatatggattatgaatatataggtaaaaggatgaagaataaaataaacatttgttgtatatttttccggacttctatgtgtaaactcacacatgacatatacaaaattttatgtattttgtagaaatgggatcatattatactcttttataaccaaattttatttattctcttttttatgttgatacatgtgtattctcacattatcattttatttttaattttttaaattaatttttgtttttagatatggtctcactctgtcacctaggctggagtgcagtaccatgatcatggctcactacaacctcaaacttttggactcaggtgatgctcccacctcagcttcctgagtatctgggactacaggcatgcactgccatacctggctaattttttgcagagatggtgttttgccatattgcccaggctggtctcgaattcctgggctcaagcaatccatctgccttggcctcccaagtgctgggattacaggcatgaaccactgtgcccggcccaaattataatttttaatagctttgtagtttttcagtgcttgcctgtattctgttttatgaaccaatttcttactgatgaagttttagtttgtgtccagtgggatgtttctggaacccatagtaaacaccagtgatcactttcctcctctgttttcctttatacatggttcctattattttcttgggaaaatttcatagaaatgcaactgggtagagagctattcaccattttaaaatctggtaaaattgtctattataaatttccacactatacttttttaaaaaatcgttctttaatgtaattcttataaatcttatagctttgtataattatgaagagaaaaatggcttgtatccctttagaaagacatgagttttaagattatggttcaggctgctcaaatttcttcccccataaacaggaatactgccagaaatccttaggtgaaaactcatcataaaggcattgggacttggcagcttttgcaggacatttttagagggcaaaaaatagagaaaaacactgaaagtcagagacagagaccagtgtaagcatcagcttttaatagagaaactgggtagggtggaaaaaaaataaagcaaccacctcatgcatgtttctttatattattattgaagtcaaataaagaggaaaatcatttcttcttcctcttcctcctttttcatctcacctctccaatggcactttaataaaacgcttggagtggccagggcactgacagacagacaggggctgctctcaaggataatgagtcaaaggggaaggagagggaatcgctgttctcgaatctctcttattctactgtgcagttaaagaggtctggacagggatttcactcctgaaaatgaggactggactttgtggcttctgttggggcacctttagagtggaggtagacttttactatgtacagacaacattgtgttggtgacatcattcataaccacctggaaatctcctttgatatgcaaatcaaacaaccataactttgtgaaatttcgactgcttcctattgtggtgtctgaggactggttacattcagagtccaccctgatgtctttgttcagttttctgctctttctagttccttacttctttgttcctgatcacctgtcaagtaaaatgtcctcagatccttttgtattgtctttggagttctgccttaataaagcatgaagaacttgagtagctcgttcatcaactttcttgggcaatttctcattgaaagacacttgggtgtctttgggtgtgcagagctgagcatggctttatgtttttagaaaaaatggctacattggcaggcagaagaactgcgtccttggaatcatggaggtcccaaggttgcatacattttgtgtgacatttttcctattcaattaattaactaatatttattgagctcccaagtgtgtagtgtctgtaggcacttgggatgcattcattaagtaaaaatcccaggctcatggagtttaaactgtagtagggaagataataagattaactaaaatatgtaatatttgaggcagttaagaataaaaaatgaagcaaggaaggagaatatgatatgttagacatcagaggagatgaagttgtgaataggcagccaggaaagaaggtgactattgagtaagacctgcagggcgtcgcatattgttttttgcctctgaaagcagttaatttcctgttaaaatggagtggatgagatcaagagtattacgtagatagctggtaaatgcgatagtgtgtaaaatgttctataaagtctaacgtgactttatgatgaaatttcttcttctaggtttattgcttgcaattttcaaaccacacattgggttactgtctaggatagtgattcttaaagtgtggttcctggaccagcagcatttgctgggggaacttgataaacagtgtaaattcaaggaccccatacagaccttctcaatcacaaaccctggagttgagacccagcaatccatgttttaacaagctctccaggtgattctgatgcacactaaagtttgagaaccactaacccagtgtcatttttgtcttttaaagtgtcttcttggctagaagctagccactttgggaaaggttattacaacttctgatgtgatcaagcaaagtaaccaactctttattgtatcttaatatgtgatattctgaatgtgtttaaaaggtatgagtttttcaggctctggcagtattttagaatgtgtatgtgatttctatttatttccatgttttgtcctatcttcttaagatagactacttattttaaaagcagtacttaagttaaaactttttatgtttctttttctgccactttcaaagtgttgaatcacagtgtgtaatgttggaactgatatttttatagcggcttcaagacaattgatatttatgtggaaacttgaagacagtaggtttatgtttagtgaaggaagtttattacaaagaggaaaattggccagttgtggtggctcacgcctgtaatcccagcactttgggaggccaaggcaggaggattgcttgagctcaggagttcaagaccaggctgggcaacatagtgagatcccctctctacaaaatattaacaaaaattagccaggcatggtggcacacttgtagtccctggtacttggaggctgagacaggaggatcacttgaggccaggaggttaagactgcagtgagctatgatcatgctactgcactccagccttggcaacagagagagatgctgtcacaaaaagaaacaccaacaaaaaaagaggaaaattattccttaatcattattgctggaatatagttactttccacaaatagtgaagtgccagttgtaaagcatatctatatgtttcctagactttggcattactttgtgaaaataactgtaattacttatgttctatgtaaatgctttccattcatttgtatatgatggcatatatagaaattataatgtttgtaaagtccactgggataaatggacaaagcagctgaaggctgaaagcaaccaagccttttacagccccttcattccccacactcccaaaaagctgagtgaatggtcgatacctccacatgcttataactcattcccagcccaccagtgtctagcatatctggttagtcttagctttatataagtgcagttatttgtgaacttgttttaagtattggaatacaatttaactttcattcttattttggagaccattatttaaacagatttcttttttcctgcaaaaacactcttttcacaatggacagagacacggtgattacattaaaaccatctactctatgaataaaaatgttaaaaccaaaatcccaacaaagggttaataaaggcaaaaaaaattggaaatgacatgtgttttaaagaaataaacatgaattatctttaagctgtcaatgaactataaattatgtgtgctcttgtatatgctttcctgtaaatttggactatattaatattctaaagcttatggtaaaattatgaaaatatgctttcatatctataagtaacattttaaaaaatctcagttaatttcagaaaaatactgttatcaaaaggaatacacctgaatgttttggagttaatgcagaagcatatacattctcctgaagtggctgaaagtggctgtaaaatgctaaatcatctttttgaaggaaggtaatatagattcattaacttgtacagaatatatcatattgggccaggtagaatatcaatatttcaagcatatttctaacaatgaaaagaaaaagaaaaacataagacacttgaaaactgaagcattttgcaatgtaatctcgtgtcactagtaccatagacttactttatctgaacactgaaaggaatggcaagattgtggaaacatgttgaaggtttgcttttgaacctgatgcttgatgttgactatattttgaaaagtggtaattgtatagcacatagcatacagcagtttttctaattattgtgtgtgtgaaagttataaaagataaaatcagtttatggctaaattttgctctttcacaacgaatatattattccttcatctgaatgaactttgtcttctcttcctgctctcaatccttagttagggaaaattttaactacatctagtccaagtgcagagatgctaagattatatagctggcggttagtggcacaacagagaccatatcctttgatctatgtgtggatggtggtggggcagggtgggatgaggtgggggtgaggggtggtaggtgtgggcagaactctcatgtgtaaaaaaaataattggcacagaagttgcagtgaaaactaattttgttcctggttttgccactaatttgagccaactgtttcatctctaaaacttcacgttcctcattgataaagaggaatgataataacaactttgaaagttgtaagcttagaatgtaaggattaaataaattaatttttacaaagggattaggataatgcctgctgcattttaagcactcaacaaattgtgtctattgttgttatactgttactaagtgtgaataaatgaagtgcatatagcatgaaatgtagcgtaactgcagacttgtaagaagtagggttacactgtttttaacatcagtctaactaatctatgtttatatatctttctaagctgtatttctcttatttaagtgttgtttttgaacactgagatgaaaagtttatcttaaatgttgattttaatggggccggaagtgtgcaaacctttacaaatgaggcaaaaacacagcggaataaactccagtctaggattctgtagattctgggcaaggcatttaatgtttctcgctgcatgctcttacgtaaaatgtgtacagttgcagccctggagattccgcattggctctgacagtgtgtctgcccctacagaactcatgtgagtcgagagactgataagtaaacagattattataatacagtctcagaatgcagtggcagtagtgtgtaaaagacgcaatggtaagagtagagtggactcagctggggttacccaaggaggggaggctccaatggagggatgtgtttaaactgggactttaagttggaaaagaaggaatgtatctcagtgtccacagaaccatgcaaagtgagaacatggttctgatatgcacaagtttcagttaacaagcaaagcaaggattgactgtattaaagttcatagtacctactgcattctagtcaagtgacatttgctcatatgtaaaagaaagaatagcttaaatacctgagagaaaccaagactgtaaaacaaattaaaaataattaaaaataccttataagaagtccagtgatgttaatctggaagaggaaggttcgtgtgatgtaaagaggccttgtcttggggtcagacaagtttgggtaccagccttgtctctgtcactttctagtgataagacctgaatatttaacctctatctgtctaagttcttcatgtagaaaatggggataataacacctacctgctgggattgttgttattgacccatcgtaggtcagaaggatgttgttagttttatgaagtgaaataattcccggattactatgaattctatcttatgagttcaaagtttagacaattaaaattatgtatgctcatactactgatttcaaatgcattttcatatagtctttcctgataaaatatattggttctgccctcctgtacttatttcaatttggtgtttataccattgaatcagatcagtctttcaataagcatgccaattttatatccccagcaacacttccctggatataatggcagcagtggtccccaaaatactaacagttatgaaacgtcatgagacatcattaccagtgcagctggaggcgcttcgagctattttacattttatagtgcctggtaagttacatagttgattgtgggaagagataacaatttaaatggatttttgatttttcatgaaatagcaatattctaggcaaatattaaaagactagtttctgtcgactaaatgtaaatctttctgttaaaccaaaaagaggttaaatatgatgcagaagagtcacttagattaatttttataagaaagcaatatgaattcagtaatttatttatacaaagtaactacaatgtaaaatgtggagcttttatttttaaggagggtgttcatctctgataattcttttctatttttgttgccatgacctgagttcaagctttttttctcttgtttgaattgtactattagctaattttcatacctgttctcttccttcatttctatccttttcttactctatgattgaattaatctttctccaatgtggcttgtactcatttacctcaatggcttccaccctccacccacctttaattgttactgacatctgttatcaccttatttgttctccaaagcccctttaaaacacctatgtctgtattcatccaacacatttattaagtgcttcttatgttctaagcactgtgatgctgttaaactttaaaagatgaattggaaaaaaagcacaactgtactattataggagcttagaggtagatggaagtgtaaatagataattaacaggcagcgtgagagaggcgatgatagatgcatatataaaaggctttgagaatatcgatggaggcacaaataatttcttcaaaggagtaggacagagattgtaatatttgaggtggaagggtagatagagctggcctggtagaaaggccaaggaagacatttcaagcagaagaaagtgcacatggtcgggggtgagtgaggcatggaggataaggagaggagtgcttggggatgagtgtggaaggaaaggctgaggcccaaagaaaagggccttgaatgcagtgctaaaaattttttgccttttaaaaatggaagccaacccaagtttattaaaattgtttgcagattgtagtgtcacaccgaaatttgcaactaaagacaatagcattgtggtgcagaggatgatagacagggagagagactagatacaaggagattgggttggaggtccatggtagtcaagtgagacccagtgaagggacccagcaatggaaacacagatgagagggcagattggacagatggttgggagatctgtttgatgtgacatcgtgatccctaagtatgagggatgatactttgttctgtgaagttcccaatcttttgaagtcatttttgctttgttcttttaaaatgaaatcttcctaaatctatcattttctctagttatataggtattttgtttctctgtataagagtattactcaatataaaagttttttcaaggacagagctttcttcagtcatttttttctgggccccccagtcctctggtggctattcaatcagtattttaaaaattgaatcatggtgtcctattcatagtttcagcttagttttgagacgtaatgtaacagatgtaattttacttgaaaatattattgcatgttttattgaattttatttttagattgtaattaaaaacaataaaaatgccttttgattatccttaaagttgacagccttattcttttgagggaggttttgggttttaaagataaccaaaggacactcaaaaaccgtttctgttagttaataaaataatactcttttagtccaaaagcaagttttgaatacagtatttcttttcttttattgttcttaaactgatcctgaaggaaaattgttagttaacaatcaatcagatgtattatgggtgcccttgaaaataatcacttgaggactgtacttaatgtaaaaaaatacattttataagcatatcagtatgtaagtacattcttctagtaggtaaaggcttaatcatttactgtatgctaacatattatactaccacagtacaccatactgtactgtacccaacgtaatgtaccatgccgtgcagtaccgtatcattctaggtatcaacaagtaactatggtagcaatactccagtagttttgaggttgaatatgattctggtttgaacatgttaagttgaggttcttatgagatacctaggtgtacatgtattcatttattagctagattatattcaggttttttagtggcaaagatgtacattatggcaaatgtgtataatttagggtccaattaaggatagcatattgcatctagtgcatatatcttaagtttctttcaatctattactgtactttttctcttctactttataataacttgttatatagacattgagctggttgctttaagaaaataataatgaagacccagttactttaataaaatatcatttgacttgtttcttgagttactgctagttttagaggaaataatgtgaaattctttaagaagggatataggcaaattgggaagtatatagaggagagtgattagggagataaaagcacatcaaaaagagcaataagaggaactgagattgttttatttgaagaagagatgactcaaagggatatgaaactctattcaaatctacttacttataaccagtaacagtaataataccaaacaatatggaggacttaaaaagtattaggcattgtttgaagtattttaagtatattatctcttctgattctaacagcactccatgaggcagatattattattattattattttggtattgtatggactacaaaactgaggcatagaattgtagagatttttaaggtcaggcctgatataacagcaccagattttcagcttatgcaggctgactccagagtaggcattttaaaataaatatttgtgcaacaatcttgtcctgaacaactgttgtatttaaagcactatgtctagaatccttaaggtatcgggagatgatgagattatggatactgctctcaaaaaaatttacagtccacacgacctaaaggactgttatgtagaaaggaaattatatccatttgaatagtaggacctcaagtgatagaacaaagactggagaagaaatttacaggaatgttcatttggtcttaatttaaggaagatgttaaataagacccattacatgtagcatgatgtttttactactcaggccagttttagtggtcacatcttcttaaggtgtaataggcagcctcagaagttactgtggtcttgctcactggaaatgatacacagacagtttaagggacttgccccagaccacacggcaagaggtgaatgtcagaacctctttgagtacattttaaaataaggactgaaagttggaggagggtggttatcaaggctgccttccttaccatagtattcccagcattaacaaaatccttggcatgtaattggaattcagatgcttcttaaataaatgaaaagcctgttgtagccagcttacagtttgcattaatgcagattattaaagtggaagatcataaatgattttttattaatatttatgtctataatcttaggtttggaaaacattattcattcataataattttaattatatgttacattaccacatttttgacttgtagtgtttttagcatagttcagctacagtgtagcttaataaagaatatgattttttaaaatagcaatgctattatatagcctttacagaacttctaaaaaatgacatgttctctaccaccttaatactgaaactcaaatcttattttttgctacgattattccagctactcttttttgtctatatttcatttctgcctttttatgttgtggtccaagtaactctgagcttttctcatgttgtccattgttgcataaaaatcttccagcatcttaaagcacagcctactcacacaaaaaagtgattgtttgctacagaaaatttcttcaccatcgtaattttttgctacttcaaattcagtaagcattcttacacattatatttattttatattcagtgatgaactatttttatagattccttaaaatttctggttatttatttgataaggaaacatgtactagaaaaaagtacaacacatatattgtgagattaattatgacaatttctagaaagtaacagtctgttcaactcaaatgtttataagaaaattctttctttatttatttatctgtgcatttaggcatgccagaagaatccagggaggatacagaatttcatcataagctaaatatggttaaaaaacagtgtttcaagaatgatattcacaaactggtcctagcagctttgaacagggtatgttgaatataagttttctgtatttatactattaactaaaatattaaatttggagaactaggggcgctttttcagtctaagttttctgttctccgtttgctatgataggaggaagtcatgtggttagagacataagatgacagtggggatgtgggaagtgaaaagatatgtactaagctaagtccagctaagtgtattatcaattatagatgtaggcaagattcttttgattgccagtaacataaatccactctagtttgctcaaccagaaagagaaccaaagagccatatatgcagctagaccttgtgagtcatgctgggtactatggctgctgttttctctttctgtcctctggctacttgtctttcttttctggtctcatagtatatggtttagcccatgaagacataccagtgttaacagtaaagtcttcggctgggcacagtggcccacacctgtaatcccagcactttgggaggctgaggtgggtggatcacgaggtcaggagttcgagagcaacctggccaacatggtgaaaccctgtctctactaaaaatacaaaaattaactgggcatggtggcacgtgcctgtaatcccagctactcaggaggctgaggcaggagaatcgcttgaacccaggaggcagaggttgcagtgagctgagatcacaccactgcactccagcttgggcgacaagagtgagacttcgtctcaaaacaaaaacaaaaacagacaaacaaaaacagtaaagtcttctttgattccctacgctccttttcattgttctccggagaaataacctctgaaatgatttggtatacattgtttccattttttagcatttacatatccatgttcctacattataattaaagtatccataaatcatactgagtatgaaaaagagaagaagggaattacatttaaattgtgtaatgcaaaaagtattggtggaattaagaagttttggaaattttgcataagatgaattggttctttattaaagatgttaagaataaagacataattagtgtgaacatttttataaaaggaggagcctatttaaaataattaatggaaatgattccatgtgatttgatatactttgatgaatgtcataaattaattaaagtggcttccagagagatctcccttaaaaattcattttaaattgaactttatactgtcactcactgcctataatatgtttgagtcatttatactcaaactttaatacaatccttgagtatggcaagaatttatgttgtaatgggttaaatttatcttgagaaatatttgttgaaaataagtatatggaaggaaggggttaggcatttagaagataaataaatatgctttgtactcttctctcctgaatctcataagccggttgttgatggctgttgtgaaaccttggttcttttctttaaacaagagacacacagcagaggagatgcagcatcgagtaatttattgcaaaagaaaaagaatattttgcaagttaagtgaggaatagacacttataccctgacagaattcagggtgggcttactagtaaggatgagacagcgtaaattggcactaggaagactccctttgtgggagttgtacatgatttttcataagtgggtgggaagaagtgttactagtaagcatattctaggttgtcctctgagtgaacatgtgcagtagctgtacatgcttgttcatatatcgcatgtctcataagtatctgaaatctccacccaggggtgtgtgttttactattataatgagcaaagggtcagtctgaggacaaggaaaatcaaaatgtgcatgctccccacgctacctgacttcaaactatactacaaagctacagtaaccaaaacagcatggtactggtaccaaaaaagagatataggccaatggaacagaacagagccctcagaaataatgccgcatatctacaaccatctgatctttgacaaacctgacaaaaacaagaaatggggaaacgattccctatttaataaatggtgctgggaaaactggctagccatatgtagaaagctgaaactggatcccttccttacaccttatacaaaaataaattcaagatggtttaaagacttaaatgttagacctaaaaccataaaaaccctagaagaaaacctaagcaatactattcaggacataggcataggcaaggccttcatgtctaaaacaccaaaagcagtggcaacaaaagccaaaattgacaaatgggatctaattaaactaaagagcttctgcacagcaaaagaaactaccatcagagtgaacaggcaacctacagaatgggagaaaatttttgcaatctactcatctgacaaagggctaatatctagaatctacaatgaactccaacaaatttacaagaaagaaaaaacaaccccatcaaaaagtgggcaaaggatatgagcagacacttctcaaaagaagacatttatgcagccaacaggcacatgaaaaaatcctcatcatcattggccatcagagaaatgcaaatcaaaaccacaatgatataccatctcacaccagttagaatggcgatcattaaaaagtcaggaaacaacagatgctggagaggatgtggagaaataggaacacttttacactgttggtgggactgtaaactagttcaaccattgtggaagacagtgtggcgattcctcagggatctagaactagaaataccatttgacccagccatcctgttagtgggtatatacccaaaggattataaatcatgctgctataaagacacttgcacacctatgtttattgtggcactattcacaatagcaaagacttggaaccaacccaaatgtccaacaatgatagactggattaagaaaatgtggcacatatacaccatggaatactaagcagccataaaaaatgatgagttcatgtcctttgtagggacatggatggtactcagcaaagtatgccaaggacaaaaaaccaaacaccatatgttctcactcataagtgggaattgaacaatgagaacacatggacacaggaaggggaacatcacactctggggcctgttgtggggtggggggaggggggatagcatttggagatatacctaatgttaaatgacaagttactgggtgtagcacaccaacatggcacatgtatacatatgtaactaacctgcacgttgtgcacatctaccctaaaacttaaagtataattaaaaaaaaatgtgcatgctccatacaggggcaattccctactggagatagctttgcttaaatgagctggactacaatgcaaatgctgaaacttactatattgacagtaagattgccacagttgccgcgtcctgaggacatggttacttccttttaatacctatcctgtctcattgtgagaggattaacaactgtgcataaaaccagttgttctacatgagcacttaggagggataccagcattgtgaacatagtttaagtacgtagaggagggaacagttaagtttattcatggtgagtgttggtgaaaagtggaagaggtaccaaaacagccgtatagataactggttccagttagccaacattctctaaagttattagagaagcctaagtgaggtgtaacctcagcagtcgggagccaagagagcaagtaagtgctgtgatgtggagaaaatcactttgttccaactgagaagaaatggttgagcactgcttttcccccatgccagtactgacgcacagcctttcacttagcactgattatcgataggggtggggagttaaggtatggggaaacacaagtaacaatattttatttcaaaaacctctccactgtaattcccctaatccttcatcatggttgaggaaaatggctctaaaaaatgagagcaattactgtagctccaaaattctgtgattgcatgtcttactctgaatagcaattacaaagcatcagaggatttaggtccaaatattgcagacacaagaaaatgaattacattttaatacatctaaacttggagagcagagttccaaataaggtagaacttgagattcaactctgatttataaagcagagactaagaagagtatttataaagcgaatccatgtttggatacataaaagtgcaataaaattcaagctgaagttaaaatctctgtctagaacagcgatgttccatttatgcctgatccttttagcttttccacagatgaagactttgtcacctgttccagagagatatatttgttcattattgtttccagagagcaaaatggaaaataaactctgcacattttggccgcatctgtgttttatatgcggtgacactcctgttctcttcagtgaggaaatccagtaaagtaaaaccagtcttctgatgaaatgggcacaaatcaaagaacttgtgagcttcacaaaaaccttgaagcaaaatataccaagcttaaatattgaatgtattgatttcagtagtcaaaaacagagctcatctgcaaaagcaacaacaacaatgacaacaacaaattacatataagtaaaatttaaaaaaggtttacaggatgaatatacagaaaactatgaagcttaggggtagagaggagtagttgaatatatgaaaagatacatcttttttgatgaaggactaaatttttaaaaatgaaaattgatctatgtttatgaaccattagtaaaaataacaatagtattttctggaactaagtaagctagtgtaaaatttgtatgaaaaaattaatacatatgagtaatcaggagaattttaaaaaagagtgttgattcatatggcctaactccaaaagataatcaaatgtattatacaattttagtaattataatggtgtgatactggcactaggggagagagatcagtgccacagaagggtggctcaggaaatagactcaaataaaaatttgtaatgttatgatattcctcgactgcgggaggaaaatagattactcagttacttgtgttggaacaactaactcttcagccatttgggaaaagcaaagaaagctgaatatttatcttactccttttgccaaaacaaattacagatggatgagatttaaagtctataatgaaattggtatatgtacatattaaaagtttcaactataattgttattaacgtaaatgacaacggaacaccttgttggagggaaatttggtaatatctatcaaaattaaaatgccatgttttctgatcagcaatttcattctaagaatttttaatgtagatatacttgctcatgtacataaagattattagacatgaatgttcactgtggcatgattcgtaattaaaaatgtggcaacaaactaaatgcttacatggtaatcattcatgctgtttgccagatatttttatttctccaccttgtggtgattctggcatagtatttagtagttaggttgaagcatgtgactaattttggtcagggagttgtaaatggagggaatagctggtgagacatcttgcggaatactgtctcaggtaccatcgtacctgcagtgtttgagatagtagttgctccatcagtagaggtccctgagggactacaacaagcagagtcccctcctgacctgcaacacatatgtagcatgagcaataatggaacttattatttttgcaccctataaaattttgagaattgttactgcagcttaatttaatctttcctgacaatacaatacctatcaatagagactaatcagataaattataccatttccaaattgtatcatcctgcataaatattaaaaaacaatgaggtgagattcctaatgtgctgctatggaaaaatcttcaattttctatgtgtacgaatgtatttcccaggtattcgttttttccttcctgtatgttgacacattatgcacacttttggatgaaattaatatatttccaccgctttatgtcctctccctcacttttatcacccaattgtagcaaagtatatgtttgcttttatagctttagctatataaaattttctaatagctaagtttgtggttcctggaattaagatatctgaatttaaatctaacactactacctacagactatgcaccctggacaagtacttaatgtcttggttttgttatgtataaaatggagataataactgatttttcacttagagttgttgaatattttataagataattcatgaaaaagtgtcagtataatgcttggcacatagtatgcgctcaataaatgttaatgttattactattagatttaaaagtatcttttgacccctggctatagaagatgaggaaatcagagtatttgcacttctaatatctcctgttttcccacctacttttgttcaataaattaactccacattggcagggtagataatatttatattcagctttctaattatgctttctaagtttgtgtttatcttactcctaccgttatttggaagcagtcttcatctcaagtccttttgttactgttttttcactcatcttttagttgtctgaatttctttagaaaagtttaattccctgaatatttaatgtttatattttgttgttgtttttgcctttatatttggactcctaaaaaaagttttgttggttgtaaaatattgtttttttttccttgaagtgtttgtaggcatttcactgtgttctagtactgaatgttttagtggagaagtctgaggccagcttcatttgtttcttggatatttttgcccagtgctgaaaggattttttctcttgaaatccatccactttattagggtatatctcaatgttagtcctgggatatggtatcctcattctagctatacattcaacttttttcctggaaagttttcttgagttgcatctttatatacatatacatacacgaatacatatacatggtaaagatattaaggataaagataggtgctttataaataccaatttgatataattttggcatgaaaaaaagcctgtggtgcattctgagtttggaagtagaactggatatacttacttattactattgtttttaatacaaaaatggagacagggtctcactatgttgcccaggctggtctcaaactccttgactcaagcaatcctgcctcgacctcccaaagtgctgagattacaaatgtgagccaccgtgaccagcctaaatatctaaatttttctctttcattattttagctctcttctttggggctatcagtcatatgatgttggatctcttttgccttaatcttctaatttttcaaagactaatccagagaccactatctatcaccatgccattactcatagacaagggacatcattccacacatttagcttatttgtcaaaatcagagatgggatttataaaagaaaaagaataaagggagaaaacaaaacacaaattatctgggaatgcgagtctgttttcctttctttggctaatctacttacctagtagctaggaaagaggccagcatgagtatttttccattgagctggctagctcagtcattggagaaaccatttcaatatttatggattctctgaaaaaaacttggagagtaaaaaagcataggtggtaggatgctgccatccttctgttggcatctccagagtttcacttgaaaaacacctcctaagtagacactgataaattgattttaatccctatttgagcaccaatgcaatacattactatttcaagatggagcattagataactaaaggaaattctattgtgggcttgtaaatatcatgatagtcatacctatgtaatacatatatgtatatataaatattaatttttaaacatttacttagctattaaaaatcaaatgctcatatttaaaattattggtttattaatgcaccatcacatgttatcatgtgatactctgaattctctttttgcatagacaaatcagagattacctgctgcaagtcattagtcatcattaacctgtactgaaatggttagagcctaggggagttccaggtaaaaggaccagtaatgagtagaagcttgttagatgtagagatattgaggacagagataggtgctttataaatatcaatttgatataattttagcataaaaataagcctatgatgctctttgagcttgggagtagaagtggatatgtaattttcagggcgtagtataaaatggaaatgcacactccttgtttgaaaattattaacgattttacaagggtgacaacagagcattgaagcaagtgctctgtgagcatgaagccctgtgtgaaatacacacctgtgaagctggccttgcctgccaaacaggaatgctggtactgttaatcaaacagaaagttcagaatgacaatctgacttttttttgtagaagatactaaacttttggccttgaatatctgaatttaagatactggtatcaagccaaaagaaatttgggcttagaacttgaaagatttaaacttgtgctttgacttgtgtgctcacagctctctgtgtcacaatttttttcatctgtacttcgggacaacaatagtgtgtcgacatcacaaaggttttgggaggattaagtaggtgaatacatatgaagttcttaaaagaatgtctggcactgagtgagtgctacctcggttttggcatcgttattgtggtcattgctattgttattatgacttgaagtcatattagtgtatgaaatcccatccatgaatagaagacagaaagaactttgggcagggtttggaggtaaaagaagacattgtaaaggagatgggtaaaggaatgattttaaagactgagaataattaggggagtacgatgtcatggaaattaagggatgataagcaccaattaagttgttattggacttttttctttgaatggttcaaattcagaataataaggaaagaagtctgattatacgaaattaaagggtagggtgactgtggaggtagtgggagttgacttttctgctaataagtttagaaataaaggaaaaatggtagcttgaggaaagagaggagcgattaagggaaagtctctcgttaactcttgtttttcatctctgaggacagcgcttagcccaaggcctgacctgtgatcattactctctcaaggtttattccatggacagagctatctcatttcatgcttataacaaccctacattattagaattgttttagagatgaggaagctgagacacacaccaaaccagccttccaatttcactttgcacaactttgaatttctttatatttcttgaataaaagttccactttttaacttaccacttcttagcagtcattgtctaactgagtaattgttacttcattcatttaatggttctcagattcgcataatttgaacctaaatttaattggcctccaagctgatgtgcttacagaaacagtgacaggaaacaaaaatgtcaagggagactatgtattattaagatgataaatgaaatgatgtccaagctgagcaattaaagtgtgaagtagaaggacacagggtgagaaactgatgcttctcctcagcctctataaaaaagatactgaataaagataattgagaggcattaggggactagactgagaaaggattggaaatctgttcactgagagtacagaaatgaggaagcttggaaggcagaagattttggtcaaagacgtctggcttgaagctatttcagctctttggattatctgtggtggaggccatcacgtctttggagtggaggtaccatgaaactagtgtctgcaaaacatcatctaaatgaaagcaaaattcctgagaaggatggcactataatttttaaaagaaaagctatgagttaagcattcatatcaaggtagatgtttggagtgtattgcttgtgtgtgaaaaggcagagatgaccagaataagagttagaggtatgctgcgttttcttcttggttgatgagtaggatggcctggacaaagaagtgacctctagtaaaataccttcatagtgtcaaatcatctggaggaaattcagattaaagaggttggatgatgtcgtaattaagatcctgggcttttaaggtggacacatttatattcaagtcccaggcccaatgcatattagctctgttacttgagcttttatttctgcatctttaaagtttggcaaacctattacatgaagctgttgaggggataaatgaaataacgcatgcaaagcacttgcagtaaagactaattatcaatattttatttgttaagaggcagcattgcgttttactggtcaagtatgtagactctggggtgaaacatatttggtttggtttcatctctgcaatttatagtttgtgtagactttgagaatagttctcaatcattctaaccctcagtgaattcatcttctaatgggagtgatatcagtatggatttcatgagattatgaaaagaaaatgcctacaaagtatttattacaatgcctggcacagaacaagctctccttaattgtaaaaatgctaactcttattcttcataataaataaaagtaattaatgttatagaaaacaaaatcaaggatactgatttatatttggattacttgatttatattttgtcagtctataactggtcttaactaaggtaagtattaagatctcatttttaacagcgagtattcttttgattttagttcattggaaatcctgggattcagaaatgtggattaaaagtaatttcttctattgtacattttcctgatgcattagagatgttatccctggaaggtgctatggattcagtgcttcacacactgcagatgtatccagatgaccaaggtcagtacaatttgaattcaggatttagaatagatttttgtagggcattagctggtgactggatgtctttaaatatttttcttcagttttgagatttaaaacaattctttttttttattttcctagaaattcagtgtctgggtttaagtcttataggatacttgattacaaagaagaatgtgttcataggaactggacatctgctggcaaaaattctggtttccagcttataccgatttaaggatgttgctgaaatacagactaaagtatgtgcattatcttggaaagaatttgggaacttgtgcgaatttcacttttggagcagtttgtgtaattcccactttgcatgaatggggtattctagttaatggaaaaccatttatccttttgtagtattttaattatacaagcaaagaaaattggattgaatctctaaagatccagtgtttcattatgaaatctctaaagtcagcatggttattcaccatttatcttgcccataaaagttcagagaatgtgctaagaaatcccagctagctgagtttattcgcttagattttagataaatagaatttataaatattccaaagtttgtcactctctgggttttattgcaggttgcttacctttagtaattttgcttgttgatttttttccttgcagtgaaaaaatgtttttaacatttttcatcaagcaaaatttaaaacatgatatataataactgtctttgtaaggaattcaagatactggcctagagttagttcacgggagattaagaataaatttgttttgttttgttttttaattgtagcaaaacaaatagtttttcttcaagagtttctgccttggttgtggagtttgcaactttcataaactacaaaggaattttttttttttttttggagacagggtctcactctgtcacccaggctggagtgtagtggcagatttcagctcactacaacagctgcttcccgagctcaagtgattctcttgcctcagcctcctgagtagctcagactacaggcatgcactcccatgcctggctaatttttgtattttttgtagagatgaggtttcaccatatttcccaggctggtctcaaactccctggtctcaagcaatctgtcctgctcagcctcccaaagtgctgggattacaggtgtgagccaaggtgcccagctgactcaggaaatattttttgtaactggcagcattgaccaggaataaaaatacctggtctctaatctttgcacagacattatcagtaaatgagagaatatgtgtaaagttttttaaaaaattataaagttatgaacatacaaaattcttagattaataacaacaatgtgttttataactgcttttcataatgtgcctcaggctaggctgattaaaccaagataggattgattaaaagtaatcttagggaaagggaaggattttgtgccggtatggaactctcagttactctggattaattcatctaggcataaattttagaatctctatagtagagtttatgaactaaatctggcctgccaacatattttatttgtccagttcagggttttgctttgttttttgagacagagtctcactctgttgcccaggctgtagtgcagtggcgcagtctcagctcacatcaccctccgcctcctgggttcaagcaattctcctgcctcagcctccctagtagctaggactacaagtatgcaccaccatgctcagctaatgtttgtatttttagctgagatggggtttcgccgtgttggccaggctggtctcaaactcctgacctcaagtgattcactcacctcggactcccaaagtgttgggattacaggcatgagccactgcacccggccttcagttcagtgtttaaaagtttttaattcgaatgacgtactttctgcacatttgcatggcctgctctgctgtagcattcacttgttttcagagacctctgctctagaggcaggtggatcacctgtccctcagacatacataaattaaggctactttgcttatcaaatattagtattcgtagatactcagcatcataagagttcgaagtaataattttaatatttagatgacgtaagttaatttaaaatttttttgagatggggtctcactctatggcccaggctagagtgcagtggcacaatctcggttcactgcaacctctgcctcctgggcccatcctcctgggtgggctcaagtgatccacctcagcctcctgagtagctgggactacaggtgcatgcacgggtaattttaaaatattttttataggcacaagattttgccatattgtgcaggctggtcttgaattcctgggctcaagcaatcccacagtgctgagattacaggtgtgagccatggtgtctagccaattttattaatatgtaatattagaggtaataaaacattaaaaagttaagatgatccttggtggctttacccaacctaaataatactaaagtcaaaagcccaatctttcattaaaacatcacatgagtgaagaggacagactctggggatgtgcttaaggtggttctaaaaaagtaacggtgttctttataaataacttattattagaatgtaatcctcagagtgccctcagcgcttctcaactacactcaacataaatgaaatctaggagtccacactagcctttctgagataaacatttcggaagacagcgcaaaaagctgggggatatgctaggctctctagagaacctactgttcaatattataatacaaatttttactctattgacctgtttggatgtgtagttctgctgatccaaccgctttaatcctgtttaatatctgggtttcatcctataactatggctttagacaagcatctttgaaaaccaaatttgagggtattagttctttttcctgctttgctactgaatggtttgttaactagcattttattctctgtgcctgctatatttcttagtcatgagagagagagggagtatttatttacaggataaatactttaaagcaccaacccaatatatctatagttaaatgaacatcctaggtattgtttcatatacaaactctctctgctttatactgtttattcattttgcctgtaattgcttattttattattttttttcttatacttttagggatttcagacaatcttagcaatcctcaaattgtcagcatctttttctaagctgctggtgcatcattcatttgacttagtaatattccatcaaatgtcttccaatatcatggaacaaaaggatcaacaggtacagtgtttttcacttgcatcctaaatgttatgtatttatctgactctaattctcatttccactctttttagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtaaatattcaaggcctcacttttgtctttgctcagtattcttatagaatgtaagagccctgccattgtgtatctcttacttatatcatattattcttcactacagaaatttaccagtttattgcaattgtttgtgtcttgtagtagatttatagaattccagaagtaatagggtcctttaggtgttatccagtctaatctttcatttcatctgtttacttatcttgttaagttgataaataacttttcaaatgtgtcccttagtaggcatctctacaacttagtctccagatacactccacataacacatagttctaatgttttgataattttttaaccatttttttccatggttttagtttctttgcctagaaagttctcccctgagggctaccacacatggctatgcaggctgtggatggcacacttttgtcggtgccattcacagtgacatgagttgctgttggccaaagttgtgtaacactggtctttctttccttctctcttccctcctgaaccatgtaaacatatatctatctgattgttctgctctcccttcaaaatataattcaaattatctttctttaaagccctccccatacctccaaacctccaaacaaaattaagatttacttcttttgtcagtctatgaaaatatatacatatctcttgtatacttggtgagttgtgtgaaaataacagtgtacagtgttcatctttgtatcattcagaatatcgagctcattgctttacatatggtgtgtattcaataaatactaggttcattgcttatatttcagatttgtattatttgtataagtgttagagtttatactagcattcaggtagcactatgtctattttctagaaatttaatatttctaacaaagcaattatgtagtgatttaatacacattattaaataatcaataaagtactatgtttgccaatagtttactttttaaaccttactgtatttaatatccctactgtatttaatatcccacttgcctatggattgaaatcaatttgttgactgttaagattaagttaatactaattagtaatcaacataaaaagaaaaagaatttgtaacccattttcatgcattacgtttatgaattaaaatcacataaacaatctaattatttaaatttagtcaaatttcttttaagcaagcaacaattaaaatagttgctccgctttactaaagataattaaatttttccatcaataatttaatacatttttactgtgcatcttttgcatgcagattattgcattaattttaattgaaaataccgaagaactaaaaagaaacttccccttctaagtccacattaaggaaacaacatacctaaaagcacctgatacaactgtactacattccccacaggaaatcatttctactattctttcaatttatccaaatctttctacccaacaggatttttactttattcctctttccatattcttttggacttcatatgcttagttttatcttttctttttaaaacgaaatcttaaatccaaggattatgtattaggtttaaagaatttatcccagttgtcagaggttatttatatctagcaaacaataactgctgattaaatcttgtggatgagtttgtcgtatgtaccttatttgtgccagagcaaaataaggtaatcaggactatttattcatttaccaagaggttacatattgaaggactatctagagcaagggtggagttgtgttagactttctgcagagaatttgataatggaatgtacatgattggtagagaagaatatggaagtttaatactgggtatgcaaatgcatggataaaaacctcaaggtaaaactcatcaaatcacagtggaaaaagtatagtgaagtctgaataaaaataataagaggctgggcatggtggctcacatctgtaatcccaacactttgggatgttgaggtgggaggatcacttgagccaggagttcgagaccaacttgagaaacatagtgagactccatttctacaaaacaaaccaacaagcaaaaaaccatgtatgatggcacacacatgtagtcctagcttcatgcagggtggctcatgcctgtaatcccagggctttgggaagtcaaggcgggaggatcatttgagcccaggagttcaagaccagcctgggcaacatagttagacccccatgtctacaaaaagtcaaaaaattagctgggtatggtggtacctgcttatagtcccagctacttgggagtctgaggtgggaggatgacttgagcctgggaggttgaggctgcagttagctgagattgcaccattgcactccagcctaggcaacagagccagaccctgttaaaataaaataaaataaaataaaataaaataaaataaaataatataataaggctgaggtgggaggatcacttgagcctaggaggtcaaggctgcaggagctaagattgtgccactgtacagcagccttggtgacagagggagactctgtctcaaaaccaaccggtcgggtgcggtggctcacgcctgtaatcccagcactttgggaggccgaggtgggtggatcatgaggtcaggagatcgagaccatcctggctaacccggtgaaaccttgtttctactaaaaaaatacaaaaaattagccaggcgtggtggcaggtgcctgtagtcccagctacttgggaggctgaggcaggagaatggcgtgaacctggaaggcggagcttgcagtgagcctagatcgcgccactgcactgcagcctgggcgacagagtgagactccgtctcaaaaaaaaaaaaaacaaaaaacgaaccaaccaaccaaccaacaaaacaaacaaacaaaaaaccaacaaaaccaaacacttctatcatgctcattaccacctgggcactgctccaaatactttacacaatttaatccttacgacaacctacgaaaaggtccagtaggttctaatgttattcccattgtgcaagtgagaagctgaggcactgagggtttaaataacttgcctaagaacaagctcctggtaacagtgtgaaatctgcctccacagtgcctgctttaatttcttggctacacagcagattcatggtagtggtggtagtggtgttcattttctctaaaataacagtttgaataatttggttttgataatgcactgcatttattataaattagatgatcagagaaagattgcagggataagaaattatgcttttgataatctttagttatattcttaattttcttcattattatttaaatgtaaaaataaatatctgtgagcagtagtattttcctgtcatgaagctgaaattactttcataaatatgtgtgaatattctaaagagaatgactctgtaggatttaaagaaattaattcttatttttgctggcatttatttattttatcagattcactttctcatatatgtctctcttcatggcaccatatgcctaaagtcagcttggatagtttggatcctccaaggaaaattccttccacaaacatgtgcagcacacagtgctagataattaatagagaatataaaatgggtttcctgtttcaagatggtttgtaggtctgtatgtgtagggcattgacaagagagtaaaacataaatcaccttagtacaaagtaaggagtgaatggcatatcttagagaaaaaaaagttactgggctataagagaaggcatttgtgagtttttccctccctccccgcttcccttcccttcccttcccttcccttcccttcccttcccttcccttcccttcccttcccttcccttcccttcccttctcttccctcccctccccttctcttccctcccctcccctcccctcccctcccctcccttctcctttctcttccccttccccttcctcttccccttccccttctccttccttccttccttccttcctcttccacctgccttccttttaattttgctatgagcccttaaagaggattttagtaatttgctacttaaattaaatatatttgctagatgttgtgctaggcttcaggaatacaagttggattgcagtaatgtaaagccctttgcattctagcaagaaaacagatgggtatgtatgtttgctcagtgctacattaaatgaaatggatgggagccgggaggagaaatggtgtgtttggcctgagaggttagtagcaaggacttctctgcaagaaagtttgaagccaattcttcaagaatgaacacctttttgctgggtgaaaagtagaggaaggcatttggggtaatagaaatagcataaaaggtaatgaggtttgaaaaattacatgctgtgtttggaagaatgtcctggagcagcagcgttttagaaggtttttaaagacgatggtgacttgatcagagctctgtagtgctttgaggatgggttgaaggtgggcgtacttggagactggtgggcatttaattggtgccttccaaccacataaatgaatgtcccctcaaatcccttggaaacactttaattctagaaaattcaaaaattgtccccaacatctttttcctctgagttggtaccctggatctttgggtcttcttttctttccttttttgatgttttattttgggtaatgaaagtcacacaggttttgaagccagcagatttggcttcaaatccaagtctcagttgcttgctagctgtaagggacaaattatatatcttttctaaatactcatctataaaatgggagtaataattgctatggcataggatttttttaaaaaaaagattagaaatcatgtgtgtacagaatttagcacagtaactgatggatattatttctattacctgttatcttggtcttctagttgatagctccttgctagcgtctagctcctttccatagctcttcctgagtagggccagcatgcagtgccacagcttgctaaggcttctcctggattgctgagttgttctagtttttgtggcacctcacatgctaacccaccctgaacacatgctctgaaaacataacatttagaggaaggttgaagactgagagacaaggtatatctttgaggaaattcagatgcttgtcttgaggagctcaggaaagctagacacgagtaatgactgtcgtttgtgtgtggcattaataaattttacaatagctatgtccccatttagttattctatgtcacaaataaaggcaggacagtagtatttactgtgttaaggtactggtttcccaggtatcttacagtgagaagacagaagctcagaaagtgtaagcaatgtgcatatttggtggagtctggatgtaaacagagatcttgatgccaagcctgtggagctttgtctccatataatgttgtctctttcataataactgactgtcatgtggcagattattcatgctattctgacattgatggcattaatatcatcttattttcccaatctattcaaggatcagttttgccttattttattttgtttcattccaaattggagatgtagagaaaaatcacatgaagtttgatttgccagtctcctaaaaggaagaaaaatgtagatttttaatatacttaattttttttctttaataggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaagtaagtaacaaggagaatattttttacaattcttatttttaatagtatttttttaagtcactagtcttttagtggttattcatgccagtttgagggaccttaagccaaagatattgcaaaggtttggattttttttttttttggctatgaaatacttcaaaatgacatttaagttctttatgagatagcaaatagttatttataaaaatagagcaaaatagtggaagctttttgaaggggtactttttaatatatattttttattattaaagtaagatatccctgtttttaaaggaaatataaaattataaaaaagaaaataaaaataacttattttatctcttataagtaattaatatggatatttttcctaactttttatatgcttacatgtacctatgcattcaaatgtatgtaaaagcatacacacatatttatttggcatttttaacttagaatatactttatatttcaattgataatgcattttctttatactttcaagctcatgtgtattttgtacatattatgtgtattgatggtaagttaccatcttctgacactatttttatcttttgagctctctcatttgttcacactaaatgtgtttttagcgtgaaagctcccagctttccctgtgttaacttagtcccatgcccatctccttccccatggtcatcaaactccatgaatcaacaccttaaggaccatcttgcaagtaacatgtttgcttctctcatttttatgatgcactcactagcaaaacaccagttttggtcagtctaccagtctactttttccctcagtttcaccaagaaaactgagtgctgctagagaaaagtacccatccatgcaatttggtgcctttatacatcaaggtttccaaccgctcagtaggctccaaaagttccaatcaggctgaattttcctcggtttctcaaacacttcgtgtacccttacttccagtctttttccagtgttactctctctctacctagctctaaattctctcttcacctggctgtctcttcattcttcctgtctcagtgctatcaccagtctggaaggttctcttacatgaccctatagcactttatttctcacatatactaccattcaccacattatataatttaatttttcatttttataatctactttttggtaaattgttagtaccatgaagtcaatgtcaattttgttcatggttgtaaccttaccattgatactagtgttttgcacatagtagattatcatttagaattaagtattcaatattggcaaaaaataaaaattgtgtaatacattatgttgataagcatgtgtggaaacatgcttcatatattgatatgaatttaaattgtcctcttttgaggacaatttggcaatatctactaatatttttaatatatgtacatacttttttgacctcacaatgtactgttaggaatctatgatacagacattctcaatgtgcacaaaaattatgtacaaaaatgcacattaaaacattgtttataatagcaaaagagtaggaaaaaaacctaagtattccccaaaaggaactattcaaataaataatggtacatacatgttgtggaatgctttgcaatcattgaggaaaaaaaaacgtggagcaaatttaatgtcctgataaagattacattactccgtggcaaaaaaaagggcacagacagtgtttttactatgctaatgttgatgaaaatgcaactggaatatgatagttataaaagtttgaatatgaaataaaaccctccagaaatgggttccctggttgtctctgggtctttggaaattactgagacatggttagatcccatgtttcattacttaaactagtcttatgccaaaaacctgcttactttaatcttcaatatccgatggagaggaattgtgggcccattggagagggacagagggagatttatcattcactatattctctttgttctgtctggagtttttaccattgacacatcttacccagttaaaaaaacatagaattgtcatttgattaattggagggtataaccatgatttcactggcagctggtctgagtaaagaacactttgggtcatagctttcaaacatttttcaggtagtatttgcctaagtgacatatttgtgtgtgagctcatcctaccgggcttcgggataatttcccatatcataacatattactctggaaaaaggaaccatttgggtatatgggtatagtgtaagccatagtatcagttgccttcttggggtttatcatatgggtccaccacatatttacagtaggaatagatgtagatacatgagcatacttcactctgctactataattattgctattcctactgttgtcaaagtcttttagctgattatctacacttcacaggggtaatatcaaatgatcccccaccatgctctgagccccagggtttattttcctttttacagtaggaggcctaaccagcattgtattagccaactcatcactggatattgtactacatgatacagcagtatcatgtagtaatatttgaagtcattacagtagtaatatttgaagaaatcttcctggctgtatgtagaacaatgaggactcagccaacttattcttcatagtagagctaatacataatgtaatgaagttgtgagaagaatgttaactttgaaattccatcaggtttcccaatagtcataatgaatcactcagcaaactttataaaaataacaagatcctttatttagcagtttatgtgttctatgcattgtgctaaacattttatatgcatcatttcaattactctttttcatcaccatatactgtatttattatcatcttcattttccaggtgaggacactgatacccagggagctcatataactcacaaatggcatttctatgacttgaacacaggcctgtctggcttcaaagcctaggccttttcctgaataaagttagttccatagagattcagttttgctgtctacatgaaagcattgtgtacatggttatgtttttttaaaaaatatatgatctgccacctgttaattattcaggatcactagtgtaaggtgactttgaaaggaaaaatagaaatattctccagaagcatagcaatacgtaagaactttggtcctatgtatgtttatttttgcataattgttgatttctaagttgctggtgtatctcttattttcagatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggtatttattataaaaaaaaaccctttatgctttatatttacacactgacattgaacaataggacccaagacaaaaacctgacctaaatcatctggaaaaacttgagtagaaatgtgtttattatcgcaaacagttaagtttactaattttggttaaagtgatgggtcaaggaagtgtgtctctgtgcttctaaatgttatactaattggttaatggttaatattccaggaaacaaactctgactagactggaacgagattccacgctctgtcattgactagatcctttcgtggcttgtgtaagccccttaaccttgttaaaggtagtaatgtcgactttgcagggttatacataataattagaaaaaatgtatgtaaaatgtctgcaacaatgcttggcaaacaggaagcgcttaataaaaaaggtttttatctttactatagcttaaaacaatattaatattttaatagctcacttgagataactttttaaaaaattaatatggtgaaatatataatgacaatgattagggctgatgtatttagcattagcagtttggtaaaaatggagtgaggggctttcttattaatatagtatgattgaaaacactgggtgatagaataaggatatttgagagggcaaaaaatgagagttgttccaaaatattgtgtctcaagtcaaaccatttttaaaaatcaagtgtagtgatttatatacatatataatttatataaaataaaatgcattcactttaagtatatattttcatgcgctttgaaaattaacatattcatgtggtcattgtcgctattaagttatgcaatattttcattatccaaaaaagtttcttcatgcctcttcactgaaaacatccccttcccctggcccccctgacccttagcaatcatttgcttcctgacaatgtagattaatgttttctctagttttatatacataggatcatacagtgagtactcttttgtgtctgttttccaaaatgattgtactatgttctacccccaccagcagtacatgagcattctggttgctctacatccttgtcagtacttggtattttcaagttacttttagctgttctagtggaggtttaattcatttagatgtaattttcatttccctgatgactaattatgtagaggatgttttcatgttcttattggccattcttatttttgtgtgaagtgttgaagtattttgcttttaattgggttgtcttattatataagagttctttgtatattctagatagaagttgtgacaggtatatgtattgcatatttttttcccagtcatagcttgtcttttcattttactaattctatttttaacaaaacagaggttttaaatcttggtgaaatgcagttttccagtttttttcttttatggtttgtgctttttgtatcccacttaagaaaccttttcttagcctaaatttgtgaatattttctcccatattttctcttagaagtgttaaaatctcagctttggcatttaggtctaaaacattttaagttgatttttgtgtgtggtgtgtcaatgaagagttgacatttatttctttctgtatggatatccagttgttccaacattacttgttgaaaatattatataattcctcattgaattaatggaagctttgttctctttaattgactatttaggtatggttctattttagcattatttattctgtgccactgatttataccttattcttatgccaataccacactgtcttgattactctagcttaatagcagttcttgaaatcagatagtgtaagtcctctggtgttcttttaaaaaaaattgttcttattattctaggttctttgcatttccatataaatttttaatcaactaactttatgctgggatttttattgtaattaagtccatatttagattttatatagaatttatttataaattaaatcatattacctatgattttaatgtaatctataatatataattaatacataaataataatttatatagattatatctataaattaatttgagggaactaatatattaataatgagtcttttgacatattaatgtgatatatagttcaattagtcttttaaaatttctaacagtgtgttgtattggatgttttctgatactattatagatggtattgtatttgaattctaatttccagtagttcactgttgatatatagaaacataattcatttatttgtacatgaattttgtatcctgtaaacttactaagctcacttacgtgttccagtaccttattatagattctacaggattttctttgttcacaattataccatttggtaataaagacagatttgctttttcttttctaatatttatgtcttctttttttttcttgtcctattgcactggctaggacctccagtactatgttgaatagaattggtcagactgggcatcattccctggtttccaaacttagaggaaaaacatacagtctctaatcactaattatgacattatctgtagtttttcatagatgcccttcatcaaattgaagaagcttcttcctagtcatttttggagagcctttttttatcattaataggtgttgaaaaatgctcttcagcatctgctgaggtattcatatttttcacctttattttgttaatatggtcaaatacactgactgattttctaatgttaaaccaacttgcattcctggagtaaatctcacttggttatggtacattatcctttttatatagtattaaattttgttttctaaattttgttaagaattttgcatctgtgagagatattagtctgtagttttatacttccttgtaattatgggagtaatgctggcctcttgaaatgaattgggaagtgtttcccattccacaattttctggaagaatttgtgtaaaggtattttcttcttaaatgtctgatagacttcaccagcaaaggccatctaggttttaaatttttgtgaggaggagatttagaattatgaatttaataactttgatagatgtatgattattttaattttcttttacttcttaagtcagttttagtaatgtgtaactttcaaggaatatgcccatttcatataagttgccaaatttatttgtttaatgttacttatagcaattatttaattatcctgttagtgatttaattatcctgttaatctcttcaaagaatcagatttttgttatattgattttctctattgtttgtgttactttctcacggacttttgttcttatctttattgttcccttttccttacttatttttattttaattgactctttttatagattcttaatttggaagcttagaacactggtttttagaaccttcttattttgtaacagaaacatttatttaaggctgtatatgtccttataaatatcactttcactgcatcccaatattttgatgtgtcctctttctattcatttaaaaaaatttaatttcccaaatgtccaacaatgatagactggattaagaaaatgtggcacatgtacaccatggaatactatgcagccataaaaaatgatgagttcatgtcctttgtagggacatggatgaagctggaaaccatcgttctcagcaaactattgcaaggacaaaaaaccaaacaccgcatgttctcactcataggtgggaattgaacaatgagaacacttggacacaggaaggggaacatcacacaccagggcctgttgtggggtggggggaagggggagggatagcattaggagatacacctaatgtaaatgaggagttaatgggtgcagcacaccagcatggcacatgtatacatatgtaacaaacctgcatgttgtgcatgtgtaccctagaacttaaagtataataaaatatatatatatatatataaatttaatttcctttgtgacttctttgattcacgagttatttagaaggctgtcatttattttccaaatatttggagattttctggataactttctggttattggtttcagtttgacttttttgtgctcagataacctactctgatgatttctatccttttatatttattgaaacatgctttatggattatcatatggtctgtcttggtgatgtgccatgtgcaactgaaaagaaagtatattcagctgttattgggtcaaatcaataggccaaattgtttggtaggatttctcaagttttctgtgacttcactgacttttctttctatttattgtatttgatagagaaatattgaagttgtaattgaggatttgttggttgccttttcagttttatctgtttatgctttgtatattttgaagctctgttgtttgttgtgcaaacattaaggactatataacaaaggactatatataaaaataattttttaaataaagtaaccactttatattaatctaagtttgcagttagtatcattttcttcctctggaagaattcccttttacatttctagcgcagatcttccagaaacccatatgcagattactggagcactttttccataactgccccagaactcccagctatctcagtctccataaatgttgatctctatctcctcatctcaggaaagccatgagactgtttaaatttcttcttatgtatatggcccttaatttgtctctaagcagaaggccagagtgatcatagggctcacataatttgtttcccttctttcgggtattacatagtcctatgctgtctgttgcccagtgtctgtatccagtcatttaatatatttatccagttttcttgttgtttatgtgagtaggttaagttcacatttgttactctatcatgcctgaaagaactctcttcaagtatttttcaagaaaacattagaaattttaacataaaataattataaaatgcactatgctgcttgtacaaataatgtgctaatagacatttcacacacatataaagtgggataattatgataatttaataatctttataattatgtaattgttactaaattataaattatattttattaaaattcttaagcattaaaataatttaaactatagaattaattaaactttagcaatgaaccaggcaggaactgtttatatcaggatttatactgaatattataagggtggtatttggatatacagattttataatttggttttgacataaaatgaattctttctttttctaagataaagagtaggggaatttaaaatcataattaatttttcacaaatgcattcacattatttatctttaatatgatttttttattgatctttgttttcttgtaaatttattattttgaaagtactgccttttccttattttataaaacaattattgccagccaaatttattgtgtttattttaataccattccataaaagaaaccatgaaacatgaaattcaaatagaaatttattaaaaattgctgactgttaaataatttgtgtgattacaacatttaaagcaagatttgaaaaatttataagaaaaattcagggagttaatccactctctttcctatgctgctagaccctattccagcggtctccataaaaaaaattcagagaaacagaaagcagtaacagtgatcagattgcatacaaactttctgcacacacatatatttgttataacttatgtaaccgttgattgatttggctttttcctgctggactatgagctcctctgaggaatagatttattttttttctgcatatcagtatcccgtgcttacccagtgtctagtctgtaacagccattctataaatatttatgggtgaagaaaaatgttgttgaatttttaaagtgaaaaaccaacatggcttatcatctctattttaaagattttacaaagggaatggactgtgaaatttccattaataaaaataggtctaatcttccatgattgaactatgatagaaggatcttatgattgagtaagctttttgtattcaccttcatgttattttatcattttcaaaataggaagtgaaggctcatttcttgtgaaaaagaaatctaattcaattagtgtaggagaattttaccgagatgccgtattacagcgttgctcaccaaatttgcaaagacattccaattccttggtaagttaaattgtgcaattgtgattatgttgtgttttgctgctgacattctcttgataactaaaatttatgccaaagctaggaacaattggtagggatttccctgatgtatgaaaactataattttgagatttttatatatgtaatagatatgaaaacatattagatgtaaattatgctcaattcacatttgtagtcttttgagtatgcagggtatgaattttttggggcacatatatatatatatatatacttacagtacacttcaagatggttttcttctttcttttcagaactccatgtctgaaaagagcccaggctagacctctacctaatggtgtgggttggtcccatgaacactttagctagaaatctgatagtgatttctaagaaaccagacagaagtctgaaggacactgaacaagatggagtagcataatataattcattgttcatctatctatctatcatctatctatctatctatctatctatctatctatctatctatctatcatctatctatcaatcatctatctattttggtgtattgaaagtcatttaattttttagatacctttattattatttcaacctcttgtctgttttggaactatggaaggactatggcatatttgcatgaggagtctgataattctagttgaggaaattgggagccaccttattctcaggttcactttgaaagacctgttctaacctattctccaattttgattatagctgagtactaaaaatatgagggttgttttgtgttaattctagatcttaagatgggtgaaatgaatgactgtagttgaatcggttaaattagctgtcagtctttatatgctctttcgaatttatatataaatttagttataaaaagtagtttggttaatgagaaattatatggatatagctttttcactcaacctttctgtttttcagtttccttatatttaaaacaaaggagaaagagtagatgctttctaaggtcatttgagcactgaactggagttttcttttatcctcataattgggttcttagtttttacttgcctattttttcccataattataaataccattaaccctattaaaatttcatggttccttccttataaaaatgtcctcttctccaataaatgacagcaattttattataaattattttttaataggggcccatttttgatcatgaagatttactgaagcgaaaaagaaaaatattatcttcagatgattcactcagtaagtatttggatgtaatcataagtaaatagatattttgggcagaatgcagtgtttggttgaatttcctccaattattcaaatattcttggtgccagtttcatcttacataatcttcatatatatttacctaatgattccttccataagctatagaaaaatgaaacatacatttaaaaatttacctttcttgaatattatagaacacattagtctttttttttacaagttttctgaaatgtaaataacacctccaccaaggccactcttcatcctctcctccagttttctttctttcttttttttttcacaccacttacaccacctgattaaatgctttttatttactgttcatctctgccactggaatgtaaactccatagtttggttttctactgaatcttcagtgccttgaagaaagcctgattgctaggaggtgctcactaaacttttactacatgaatttaaattatttgcttatatttccattgtttgtttgtttttgacaaaagggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaagtaaatttaatttatccttgtaactttcaagacatttgaagagcttttgtatttatatctaatttgcataattaagtcgtttaaaagaacattctacttttgtgtcactgggtgataagtcccccgtgcctctggtttttgcacacatatcttagtctgtgtgatgttcaggagcatctttgagggcaggcaatggaaacagatctgattagaaaggaattccaggttctgtacggagtacatgttaaagtctgtcaagtgtatattgattatactttaatcatttaattcaagtaagacaacttcaacaatttaaattagattaggtaaactagaattagacctggtttggtagtactggctctgactcagctaccaactgtgtgacaatatgaacatgtcactccgcctgctctaatccttcattttctcatctgtgaaatagagattcaactaaatgattactgaaagtttttttttcagtttaaaataatgtgtaacttaaagatttttttctttttggtcaaagttcctgtcttgtaagaattaaagtataacatagtttgttgataggatagctctctgaaaattgactttgctcaccatttgtatgtactacagatcaaaatagttttgaaagccaaagaagatatcataaaagttaaaattattttaatgcaaatgtttaaattgttaaattctcaaggctgggcatggtgactcatgcctataatcccagcactttgggaggctgaggcgggtggatctttttgagttcaggagttcaagaccagcttaagcatcagcaaaatcctgtctctaccaaaaatatgaaaaattagccaggtgtggtggggtgcacctgtggtcccagctacttgggatgctgaggtgggaggattgcctgatcctgggaggcagaggttgcagtgagccaagatcgcaccactgtactctagcctgggcaacagagtgagaccctgtctcaaaaaaaaaaaagtcttgttgcaaatgcatttccccctttttaagcctaaaaaattaatcataattttgagatgttttaaaggcaacattacataaattttaagtatatttaagggatgttttttctctaaagtttttatatctggagacagagagaagaaagaaaggtgacccactcctccagccatgccctaatgtctaaaatgtgtctttctctcttctccacttctttgccttgctaaatacttcaagccacccagggctcaatttaactgtcacttcatcactctctgcttctcttcctttttccttctaccctccagccaacctacccacctagcctccatgtccataacacctgatgctttccacctaattctcattcggtcatccaatttgatttaaaatacctttgcccccactagactgtgaactctttgaggacaggacttgtttcaggcttgtttctgtctattcccagtgcctagtgagatctgacatatggtagaagtttagtacttactgaattgatttgtggaggaataaatgtctgaaacttggtaatccttcaattaatatttgttaaatgagcaagcaaaataattttgggatttagtctagttaaaacaaagagaattggaagagactgtgacaaggtgagacatgccggcattcaatgactggacaagctcagaaccttctcttagggaaatttcaaaatgacaccattagatggcactttgtttgtttgtttgttattgtcaaagggtctcatctatgttccttttataggaacatttcctgattaaaccttgggaataattttaaaatctttacttcagaataagttaatgagggtctgaaacaaaagcaggaattttgaaacaacttctggggctaagagtggttaataagcctctataatgatatcaaactctagagtttctcgtgtggataaatatattgataaataaagaagaccatagagaagtgattgattttggtattttagctctttgagagtattacgtacctaagttttaaaaaattgacataatgtgtaagtaggggtttgctattatcattataaaattagaaattgcttaaaaatagaaagtagaaatttgaaacaaaaagtttcgtaaaaaacaggaggttctaaaatgaaacacattataagtaactatttttatagttaaatcttaaatatatcaaaatatgtaaaatttctgacagcatttaaaacatattcccaggattatattgtactttttgttaaatcattaattcaaatatttgttgaggcgtattctgcttccattttgctctttctggaaataatttacaaaaaagctgaaggaagctttcaactctatttttgtgaacctgctttttacaatctacctgttgtaattttcctggttttacccatgctacaagcagagacgattgggccaattagtatactgaaattctgttgtggattgtgttttcaactttttgaaaattcttgatggttctagttaccagaggtgtgtaaggcagaaatattagctagacttaagttcctcagatggttcactttagaattttaaactattgtcttttcagactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaagggtaagaaagagctcattaaaaataaaagggttgcctaaatatgctgatgttaacaaaatatgctgacatttttatagcaatgagttttaacaacatggtgaaactccatctctactaaaaatacaaaaattagcccagcgtggtggtgcgcaccttataatcccagctactcagaggctgaggcatgagaatcgcttgaacccaggaggcggaggtcgcagtgagccgagatcgtgccattgcactccagcctgggtgacagaggcgagactctgtctcaaagaaatatatatatatatatatataatatatgtattataatatataatacatatattatatatatttattatatataatacctatatattatatatatactatatataatacttattatatatatactatatataatacttattatatatataataaaagaccgaggcaatgaatattacaacttttatcaactgacttacatttttacaactaatttttaaattaatgagtcctctttgatgctgttctttgaaagcaaattgtttttgatattttttctttaaagcatatgaatttatgcaatttaatcattatcttgtctcttgtgactagaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttggtaagtgttctgtgtgggtctcctccttaccaggccctctaagttgtacaagatgagtcatatatggaccctttagttgtggatttaaaagtggcatttcagtttaaatattatgctggatttaaaaaataaaattagcaggttggcaataaaacaaaatgctataaaactatgaaaagacatgaaagaaacataaatgcatattggtaagtgaaagaagccaatctgaaaaggctacatactattcgttcccaactataagacattctggaagagacaaaattatgaggacattaaaaagatcaatggttgtcaggggttaggggagggagagatgaatagataaaggacagaggatttttagggcagtgaaactgttttgtatactatagagggggatatgtgccatggtacacttgtcaaaacccatagaatgtgcagtataaagaatgaaccctgatgtaaactatggactttgagtgataatggtgtgtcaaatattggctcattgattggaacaactgtgccaaactaatgcaaattgttaataataggggaaaatgtgtgtggggtggtggtggtggtgagggaagaagggatttattgaaagtctctggactgtgtgctcaattttcctgtgtatctaaagctgccctaaaaatagtctataatttaaaaaaattatcacatttttattgtcagaggttaaaatgatagttacttggcctactgtgtagtaccctgtggttccctttagtcttaaactaaacatgcacatggctgcctgagctgggtaaggcatcctgatactgagatattgtttttcatactgaagtttcttcagcaactttttgtatgataaatatgattactctttgctgttgttagaaataaaattaatcatttaatggttttcaaattagtgaagttaatgtatattcattcagctcttgtgctttgcaagacattataataatgcaaattattatcatcacttttattaaaagttgtagaatcccctgccttctcttagcatatgaaataatagaggaaattatgttcatttgtatcctaaatgaacattttaattttaaggaacaaaatacttttatgacaataaacaggaattccccatattttatcttccttcatagagaatactcacatcttgccacccatgtgtttattctatacctgactcagtaaaataatttttaattctatttaatagcagaatatggcttaactacttattaatagtattttaatttgtcagagctttcagaaactaataatgatcaaagccatcttaatttggatagtattttcctccttttccgcgccctcccttcctcctaccctcctgaatattggccactttccaaccatgatcttgaaactggggatagagttgttgactcactcactagtaagcaaagcaaactcggtctctgtccttaaggaacttgtgattaagttcagagcacagacaaacattaagtaatgatggcataaataaatgtaattttatattgtggtgccttctgtggaaaagtttcaggatgctctgtggtacctcaggggaatctgattcagactgtggagtcaggaaagcatttctgtgcctgtgacatttaagttgggtctggaaagaggagcatccaaaaggaccttgaagcttgtgtatggtcagtgtgtcatctcagctgaatctcacaatgatcctaatggtcatacctaggacaggtatgacctctgcccaattgaggaaattgaagccgtgagattttttgtgtgatgcttacaggtgcatacaattaattatcttgttgtgaaagtttgaatggaccccagttcaaaatctgtttgctttgcactacacaatcttaatagtttgagaagtggtcttacatttgagaagatgccccatggaggtgacatctgagcaggaccactaaccattaaaaaataatcccatgtaaatggtgaaaccctgtctctactaaaaatacaaaaattagctgggtgtggtggcgtgtgactataatcccagctactcgggaggctgaggcagaagaatcgcttgaaccagggagtcagaggttgcagtgagtggagatcaggccacagcactccagcctggcgacagagcaagactccgtcaatcaatcaatcaaacaatcaataaatcccatgtaaaataaagttttagttctgtggccttatgagtgttttccatacagcatatgaaactcaagactctgaagtcttaagtggagaatcatttcgattcatttattttgcgaataggtgaggtataatagctatctttctgcttctcaggaagacagcttctaggagtgtcctggaacattttgacccttgaagattgtctagataaagaataacccatatttttaaccttgaaaatgctaataactaattcctcaatccgttttcctagaaacggtagatttgtagatctctcaagtgtcttagtgttcatctgatctagctacactcttttacagataagaaaagagactgtgtgattttctgtttcccaaattgtgtttcacagaattttatttcttggaggtacctcatactgcatttccttcttaacaattcataaagtaggccaggcacggtggctcatgcctgtaatcccagcactttgggaggccggggcgggcagatcacaaggtcaggagatggagaccatcctggctaacatggtgaaaccccatctctactaaaaatacagaaaattagctggacatgatggcacctgcctgtaatcccagctactcaggaggctgaggcaggagaatctcttgaacctgggaggcggagattgcattgagctgagatcacgctactgcactccagcctgggcaacagagcgagactctgtctcaaataacaataataataataaaataataaaaaataaaaaaatcataaaatataccagagtattgagaactcagatattttactttattttactgaatgtttcccaatctcttaagattatatgatcgaagaatacttttattaaataacacccattattattccatagaacactctttatgagctagtaccataaagaatttttgtccttataatatactgtattttagtaaagtgttcatagttgttttgctatagttatttaagagtaattctagttaatctatatggtatataataacatcacacataatttgaaatgaatattctaggaatttttactttgtacaaatgttgggtagacaaatttatatttaaatagctgtttagcttccaggttaaaaggttctggtaagtatagcatattattttattgtttagtggaagtattaaggctattaccttatttttaaaagtggatttttaaaaattgtcagtgtgagaatggaaatcaaattaagtgacacactattggtagctgttcttatttttgaatttaatggaaatctggttaaaatgattaaaatgtttatctcattttttttcttttagctgctatgcctttcttgcctccttctatgacaatcctaaaattatctcagaacaaattttcctgtattccagaagcaattttaaatcttccacagtaagtttattgttattttaattttaaaagcacattagctggaacagaacctttagaaacatgatttcgatttagtcatatagaggtaattgatttctaaacctactcaacttgatgtttttgtatgtatgaatgattttcactagataaaagacccaactcattacttaaaatggaaacttttatatttatttagtggatcattgtgtaaaaacaacttaagattgtttaattaattgctgtattagtataatgaaatgagattatactggctatcactttaacttttaaaattttaattgtttgtggaacttgatatgttgccaaaatacccttaactttcacattatgcttaagttatgtttgagtgaaattttgggggaagattaggcaagtttatgtagttccaggtttttgagatattttggttaattcatgaaaccagaagcttcctgttaactttaaattcaggcattaattggatcttgagtgtgttgtaatcttaaatgctattctaattatatcatacatgataaaccaaattcataaaaatatatgtgtaaatttatcttttcctttgttttcttgctgtcagctattccttcaaacactatggctttttagaattgacactaaaatgctgcttgcatgatgctgcaatgagctcttctgtgagcttatatttaggcaaataataattagaatttagccatagagagtgttacacaaacctataatagctaaattacgtctagctttagaatgtgtttaactgttctaactactctacagcggttcatctctttaatcttcctaataatgccctacgatagccactgttattatctccacttcataagagatgaagtaacttgcccagagtcatagttcttaaacactggtggttcaacttcaggctctcaaatcacatgcatactcatatgtcaataatgctagttttgacgttacactttattctcaccctggggaaaattatttgtgatgttatttcatgtattttggaaatactcatttggtttatgtcttttctgtgtattgttttagcttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagaggtaagacgattattgccacttaaaaaatatactttatgatttgcatcattacaaattatcattttaagtgatatttagcttctaaataccaatttcatgaaactagaagcttcctgttaactataaattcctgtcaactataaatccagatttccattaaatttaaaaataagaacagctactaatgatgtgtcacttaatttaatttccattctcacaccgacaatttaaaaaaatctacttttaaaaataaggtagtagccttaatacttccactaaacaataaaacaatatgctatacttatcagaaccttttaatctgaaagctaaacagctagatataaatttgtctacccaacatttgtacaaagtaaaaattattagaatattcatttcaaattatgtgtgatgttatcatataacatattatagattaactcaaattattcttaactgttacttaactatgacaaaacaacttagaacgctttgctaatacacaagatagtataaggataaaaattctttatagtactagctcataaagagtgttctatggaatagtagtggatgtcatttaataactataaattcaaaataagcattgtaaatatcaataccattcaatttttttttgttttttaaacaagttgtaagcctaccctatggtaaatggatatggtaacacagcataatttcctcaaaaaattacttttgtgatatacttttaaaggattatatgaatatatacataattatagatgaatgtgatgctgtgtgtcattgtatcaccaaatctctgtccaatctgttaacagactcttaaataaaccatttttctcaagttgttactggcctgtatactgtattacttgtttttcagctttccttggtacattcttaaatttctgcattccgccaccatgctatcaccctaatggatcaaccttttttgttttgctccattctctctgttgtaaatctgaaattgataatttgtttgtctcagaaaatattatttttcaagttctagcgtattgcctacaaaaaccaaaagaattaagtgtctgacactgtgaggttcagcaaaactgtgcatatattttgctacctgattttttgccagcaaatgagtgttttctattataaatatagtatatattgcttaaaaatttggaacagaaaagaaattactccaattaggatgccacctaaagtataagcatgtagctgtactttgagaacactaaattgcatgcaggtttgtagtgactaggtcttcttgcctttactgaaggagcagaatgaagtcacagataatggataaccaaatccattttgtggtaagaacttccttactttcaatgtcttgaagagatgaagtatgttaccaaaggagattgggttttttaatattacagatgagtgacatagattgtttgggagtaagtttttatatgtaagtttttatgtttttaaacacatactgacaacttatgacaaacctttggaaagttttaaaactctgttgaaaggttgtgcaagctgctgatggaatctgtgagcctttcttgtttcttatcacgttttttggcagagcacatttcttccttcccaccaacaggttttgcccttttttttcccattaagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggttagataatttttttctatttggttttactaaatttatttcagattttctactctctgtgactttgatggacatatattgttactatttagggaaaaataaatagtaatatttggcattaatatgctgtgtgtcatttgcctttcatttaatgaatgtgtttctgtggtgccactgtagagatttctcattcttcttagccagactaatgttgagagcggcttctcctccttctgtttcttttcagtggagtagactctaaaagaaaataagtattgctatttggtctctggttaccaattacacaatctaaagaaatacagcacagtataataacttctcacactgtatttcatatagcaactagttaacatatgcctcttacatcttaaagcattatagctactgacatcatgtgaaattactaacttctattttgcccattaggatgagtaatctactcaccttgatcagttttgaaagcaccaaaacttctcaagtatcactgtttctggtctttacactttaagcactttaaatatctttggtaatggattttatcctcctttttgttccctttcagcacatcggtcttattactttctcataaaatcctttgctcccttttccacagttactgtattaacgttgcagacctcagctctgtcatcacctctcaacttgactgtaatatccaccaaggcagagaccatggctgtgttcactcactattcaaatcttggcacccaacacagtgcctggcatacaattaatagttgtttaattaccagtgatttatacttactcattctcttctgcctaaaatctcttaaatttatatttaacttcatctgtttttatgaggaaggattttgttttctgaactcctgagcttgatttcattttaaaggagtttgttatcttttgtgctaattgtggctacccttcatcctacccaattatttttctctcttgaaactggaaaagatggtcatataaaaattggttcagttcttactaaacatttagtagaactagctttcagtgtattatactgtattatctaactaaatatttttaatatttaatatttaatttaatatataactaaatatttttaaacatgtttaacattttcagaaaagacagaaagacctagagcagattagaaattgtaggcatcatttgctttttgaagaaagacattttttcaaatagtggtgcattcttaagaaataaatcaagaaaggtaatgttgctttttggtcatatcatcaggaatgttggtcagattcttattagttacaggaatgaattgatcactactctgatgtaaaattcacttatgatttagtctttttctctaatttgaaactgtggcaacattttaacatatttcaaaatatatctttctctatccattatatttttgataacactttgactctactattagtttaaaggtggttttttagctacctaaacacttctatttcattcaggttttacattaagatcattaggaatgaaagctaacatctgctgatagtataatagtttatatttatttatgatgttatgtgatctcactatccatatatactattatatgcatatgtgatatacatgaatatatagctatacatcatatataccatatatgaatatatacacacacatatataatgtaactaatatgaccctattatcaagctttaacagtatacatatatctctaccttgtttctatgtcatatggactttgtgaaattttgaactttataatttatagggtttttcttttcttttcttttctttttttttttttttttgagactgagtttcactcttgtcacccaggctggagtgcagtggcctgatcttggctcactgcaacctctgcctcttggcttcaggcgattctcctgcctcaacctcccaagtagctggaattacaggcacctgccactgttcccggctactttttggatttttaatagagacggggtttcactatattggccaggctggtctcaaactcctgacctcatgatccgcccacctcggcctcccaaaatgcagggattacaggtgcgagccaccgcacctggcgtataatttgtagggtttttcatactatttaaagacattagaatatgtatacatgtatgtatatgtgtgtatatatagaggtatatatatattgcatatcgtattctaattagtattgcaaacatattttggccttttgattatttctggtgatagtgtaacatgttttctttggtgattttaccaaacattatcaactaccctaaaatctctagcaaaatatatgcattaacagtactctgaaagacatgtacattattagttatatgagatatgcactcttctggatactatattttagaatagtgtgacatgtaaaagaactcacctaaatctcaagtatacttttaagcagtttattattttatttttatctttcaaatactaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtatttctttctatagaattttaaaattcacttttaccatttgtttggaacagggattcaaaaactgagctttctgttctaatatccagaaacctggtagactgtatggaattattccaaagcccttcatttctcctaattttacccttgcctccagaatggagaagaacatggagggatatgttaggaacaatttggtgctaggtactttgatcggttgctgacaaatatgctaaaagtggtcaatcctagtaaaaacccagaatagttctctaaacatggtctgttgtttttctcttattagtatgctaaataataaatagtattattctcccagatttttttttaaaaaaggattcttgcctgtcgtttgaaagattaaaaaaatttgtctctaatctttatttaggtcgtgaggaattctatagtactcatccccattttatgacgcagcgagcattgtaccttgctgtctatgacctcagcaagggacaggctgaagttgatgccatgaagccttggctcttcaatataaaggtgatttgttctgatcatttgaaaatagaaaataattcatgtgtctgtgtgcgtgtgtgtgtgtgtgtgtaagttaatttattttgggcaaacaattgcttcagtctctttaaatactttcttaaaagaagcactaaaattttgaattgggaaactttccgagtaatgaagtcataacatgaaaattgtatgttccatgttggtgaatgttattggtaacctgaaactcttttatgctgtaaaacttgaaaatatatatgttcaactgttttttaattatattatttcttaaatgaaatctaaatttttctaatttaaaataagctatattaaagaaaagcaatctatatatatatatctcatcaactttgtactcaggggccatttagtgtgaaattcttcagattgtatcctttaagtggtcccagattattatgctgttacatctggaatctcccttttgttgcttttctatcttttcctttgttgtcttgttgtcagctattccttcaaacactatggctttttagaatggagactaaactgctgcttgcatgatgctgcaatgaactcttctgtgcataaagtccttaaaaagcttgtgtcaggacatttaaccatgtaattggctgcatacatgcttgttttgtaatttgggtattttttaatgtttcttttattaacttttttacagctagccaacgtgagcaaatagtacagtggcagtcatatttgcttgagtggcttttattctttcattgtagactccaaattggttgactttaaaacgaatttagaagattaaattcacagataaggaagagaaaatataaactatatgacgttaatttgatataatttgtgggtttatgaaatgcttattttatttaggagtgaataactcatcttaaggcatgaagatgggaaaggaaaactataccactaccgttatatatgccacctaaaagggtgaagaattgggttaagaaaggccaaaaatgactttttaaaatgtcgtaaggttacatttttttcttaggtttaaggaaaaaaggacagttgttcttttcttcttctgaagtctgctagtttctcttttccattcaagtgaatgtcacggaaagcaaatatcaacaggaatgtgagcaggcccagtttgaaagcaaacacaagagggttttgtgtctttccctccaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaaggtaacatggtaggctggtagagaaatgtaatttattgattctcaactgcctagaaatgtcagaaattttgagaagtgagcaactcacttaaaattgtgggttttctttccttgttgctgttagcattattaaagtcctttccattttaaaattatttatgccagacttcatttctaattcatagaaatgggaacaaaaaataattagaggaacctgagagaaactaagagaccgtttctgggatactgagaaaatgtttctgagagagaatctgagaaaatgtttttgatgccttttctgattcaacttcttatagtggtgattcaatcacaagggtaaaggtgaatactgaggtcttgggatcatctttcttctattattctttaactgttatttttccatttcctcttttcttttggaattcctgttttatggacatcttgatcttttgtgccactcattcatgaattttgtcactgtgattcccattccaatttttttccctccgtattgtgaggcagctgttttatttagtcatgaagaccactaacttggttttcagcagtgtctcactaattacttagttcatacaaaatgggctttttattttaggaattatgttttaaatgtttaaagttatcttctcgtaagccaaatttttataaaatgtaaataaatcagttatcagagagaacacttttttttttaaatacttggcagaaaaaagaaatcttcactgggtactacagggagtgtggtgtaaactgtactgaaaaatacccttgatagttccatatgacaaacataatgatgaatttcacttagtctgtcttggcttagctcaatagcactaatgatcaagatactggctgataaatagagtcctatttggcctgggcagtcccagcataattatgtaatagtgtcccactatattctcaaaagcattccaatttggatgataaattatatagtcaccttggttataactccatgctggccagttagcttagttctgttccatttatatagattatgtgtgcttcactccaaaacctaatgagccatttgtaaaagtgatggcttttgcggtgcccagggagagaatttgtatgtttgtatccttcaacacacatttattacagttattaaaaggttttattgatgatagatggtaatgtcatgtaaaaatgacatattatttatttgtagactttcctattctcttgttggacatgtaattagaaactaatatgacttaaagaaaaacaaatacacaaaatttattcatccaattaatctcttaatccaggtgttttttttttctgagactatacccatacttcaataactttgttgttactgagaatattttgagtttccctttttgtcattgttgtcagagaatgtatcatatctttaaaaagacttgttggaggatgagtttgttttgaaaaggcctgaatttagttgatgcaaagtcacagataagatggttcattaagctgtattaatactgcttttgtctaatagatatcattaccaataagtcagactagtttttcttttggcacttataaatcacctttgaagacaactttttacaaggaaataaaacaaatgctttgagaaataccagtattattgaaagaaaagtatatattgctaatggatgcagcattctggcataatggtttgaaaactcatttgattgctttgtagaagaatgactctttcagatgacccagggcctgtgagcctgccagaacttgaaaattctttcttccctgaggtgcttcaacctgaattcaaagagcagcttttaatctattagagatcattttttgtcctctcatttatttttcatatttgcctttgatcttagctcttctctaatctttttctgtctcaaccttattaacaggtgtctgtgcagacacttttaagttttgttttttggctcagcctgtcagttaactgataatcatgctgaaaggagaagcaggacaaaacagagttcaatgctgacaatactccttttaatcttgtccagcccattagcagagcaggcatctctgtgggccttgagacgtagtcccgtaaaactcatcccgtttctacttgatttgctttctttgagaactcttgtttatttttatatggaggtttcctgccttggattaaaacataaacctcaatctgaagttcaatttcatcttaatttatgaacgactaagagagggaacatgaaaagtggaggttagtgaaattatctctaattctctgggttaagagatacatgaaaacagtctcttgagtaaccatttgcaggtaaatatggaagtaatggttatggttgtctctttaagtttttagtcacaagtagaaaaagaccaagttaatttttttctgtgtgtgctgaatttctatttgtagtaagtgtaagaatttaagcagaaattctgattcgtattttcagataaaaagaatatgtaatttccataggtccagaaatagggagagtttgccatctggtggttcttaacggcactctggatattattaagagttgcatttctatttaaaattatattttaaaaaacgtttggaagatacttttattgtagaaactatcctcttagggccattctttaaaaaaatcttattttatatatttctcattttgttgatagtgattagattctaagagcaacagaacaatgatcatcctctcctatcagaatcactgatgtttagatgatttctcattttcccaagttcaaggttccatgaaaaacatagcttgagtgggattttatgtctctgcgtttcactgttgatatatatgtcctcccaatataacattttacaaataaccaagcacaaaatttaatattttaccttgaatatttaaaatataataatatccaaaagctcttgtaatttgtactgatatcttatactagcgtgtctgtttcacattaagtttaatgtcttaggatataaaaaatcttttttatggttagtgatttatcttgtttttttttccatggaatttctggatagcgagataaatatttccatactattttatttgatatttccaaatttgcctctgaatcaacaattttcctattttaatttcattgtacttgttccttacaacctaaatagctttttattatattttgattttatttaaaaatgtacttctgaataatatatctgtttctgtaaaaactgttagcactgaatttgccaaccatttgacaaatacacaaataaaatagatttttacggcttgtcatttgtaatttcatagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggtttgtgtttttcgttccttattttcaaagctcagctgtagtaacttataaaagtgtttctgaatcttttatagaatttacattcaaagttgagagaatatccatacggttctttaataggccactgatttttttctttttggaagatcatcatgtgtgttcatgacaaatcatgtatcatgtcataagaaaacaaatttagaaatcacctaggagtaaagcagtggaaagagtccctgagtgggagttaaaatatttgggttctagaacttgtctttactattcaggagctgtggaaccctgaatagtcaaatgacattcataatgtcaaatgagtttagtgcatgtgaaagttatttttatattgcaaaggggaattattgttggcatggtctaactgggacgcttggagagtcaatggctccctgagatgatgcagcttctgagtggaagatctagctctcttgcatcaaatattgatctcaaagatgaaaattctcaaagcaacttcagtgctaattgtgtacttgatcatattaccttgctagaaatgtgtgagttgtttgatagtactagagtaagtgactgggaagctgcttttgatccctagattctgttgtataaaaaatagcttcccgtggtttatgatctgttccttttccccatcgttcttaaggtatgctgagatatgctgtgtttcttatctgtatttgaaaataaaacatgtctttgtagtgtgtattcagcaagcgaaacagaaaattatgaatttctacttatgtgtgaaatatgctctgtaatgcatgtcagtgtctcaaatatgcttaaatatgatcattttatgtagtttaaaaatactccattataatattggaactttagaccataggatgcacagcttctagtcccagctctgtcactagctatgctgaaatttcttcacctgcaaaatgaggaagttggactagattttttctaaagccccttgatatttgttctagattccatgtttcactgtttgatgactttttactacaggagtccttcttcattttcaagacccagcactgcagttaagtgacttgtactttgtggaacccaagtggctttgtaaaatcatggcacaggttggtgtcttttatttttgtggcacgggggttatggtcaaagcatagaacagatggcgcccagagcattgagcattttagaatttgggtttagttaaggcagaaacttttgtgaatttggaaaactgtggaacatttcacatagaagactacttgaagagcttcatggaagaaggaaagatgtcttgagttcacttccatgacttggttttcaagccacatacagatgtttgtatcactctgccccatgctgctttactagatcctgatgatgtcattggtttggttactgaattagtcaattgaatgatggctttgtggaaatccttggggtaaacacatataagaaaattaggttgctgagcctgtgaaacctctatctagataacatggaggtgagttttgacttaagtgaaatgatctgagctttaaatgcttacgattttgaaaactttggatggccttggttatagctatttttttcttatatttcacatggaaaatgatttttttctccaaatgataatccattaccaatgagtttaattagttataataatccatctctgtagctttgacataaaagaccatttgagcaaaacatactacctcagggcttttcaaccccagcatgatgacattttgggccagataattctttgttgcacattgtaggatgttagcagcatttttggcctttatattcgagacgtaagtagtatcctctagctgtgacaagcaaaaatgtatccagacattgctaaatattgcttggagaatgtgaaaaattaccctagttgagaaacattaagctactgatttgttgatgagtaaaatttatagttttgcatgtggctgcccgagttcctaaaattattatatatttttatgttagaaatatctcttccaattaaaccataaaggtaattaaattcactcaggcagccttgaataattgttcctaaattccatctaaggaaaaaaaggaagctattgtgaagagagaactcagttgaggctaaatcctgtaccatggaactcaagagcatattgaaacattgcaatcagcaattatttgcagtgtgtcagttattactattttggtaggtatttttaaattagattttcagccttctgcacatatgtcatggataatgtgattttactcaattattaaatgataatggagacagtagtgtgacccagagcacttacttgagcatcagcttgacctacgtttcagtctctttaattacttattagctctgtgaaatttcttaatgcattaagcctttgtttacttactttttaaataaggaaaataacaattatcttctatattgcctccctggttcagtgtaagtgaggggtaaatgttagctaattttatattggatctatttggcaatttaaagaatgttaatcaggaaattttaaaaaattcagaactataaagaggtacttacgtagttttggaaagtgtgtcatgtatggggacaaataaaaaagatgtgtaggtagctgcatcctgtacagcaaaggaagttttaaatatatccagcaattttgttgtcctagctggcgcacaatagttatcaggaggtaactcaactccacatagtcaaggaaaagctaaagttgctctctaaagtggtgtgtttccatgtcactatggaacacttgaagttgcacacatgtgaacattaggatgggtatatcttatacagtagaataaggaagaggtttgcatcagaactccccttttaaaaaaatgcagattttcactatgactgcaataaaattcctgaagattctgtggagtaattaagttgaaactccatgaaagttcttctcattagcatagttataaatatgataatttaagtaaaaattaagttaatttgagccactcaaagttacttttaaagacagatttaaaatgtcaataaaatgataatttaaatttccgattaacctaaaaaagaagtgccatcatttttatttatgccaataaattgaaatataatgtcattttatcactaaggtttaaaggaaatgaaatctctaaataatcaagtgaaaccaagagcaacttgtctgacagctattagcaaaaataaataggagtattcaccttcatgaatcaaggcaagggccggaataatttcatggtgcagaagctctaatgagcccacccactctatgcgccccgagctgttaggtcactaaacttattaaaaaaaggtaccattaaggcagggagaagtttacaagactcatttaactgtatgataaaagagatatgaaagagacctattcaattaatcaggtggaacattaaaaagcttacatggcaatttaaccttgataaaaatacatgggagaaatacaaaggaatttggaaaattctctttccttgaataaggcatcagttagctattcaggttatgaggttgaaggaatgttaggagctcttttaaaggtgataaagtcaagataatgttgcagattttattcttatgtaacaaaccccctcgaaacttggaggcttaaaatgtgaacaatttatcatttctcgttcttctgtggcttgactgggctcagctgcgtggttctgctccacatggtattggcaagggttattcacttggcttcattcattaaactgagctggaaagtgcaagaaaggtacatgcatgtttttggagtattggtgcttctccatgtggcctatcatatggctaagttgggcttcctcgtggcacggtgatcacagaataattagacatctttcatggtggctggttaccaagagaaatgaagcagattttttctgtcctcttaaaggctaggccaaggactggcaaaaatattaattctgctacattctagtaaccagagcaaccacaaacctagctcagattaaaggggaaggaaaagagactctatatgaatagcacctatgtatagggatggaaatgatgtgtccatctttggaaacttccactataaatagtggtagcacgctatagatccactaggaaaatcaagcacaaactctttaaaaaataagtgtatcttagtaaaatagattagaataactagataataatggctaacatacatgaggttaatatgtgcttttcaaagattagctcatgtaattctcacagcaacctttccaaatggtactttattagcccctatgatacagatgaagaaattgattgacagagaggttgaataatttatccaacggtacacattcaggaagaggtagagttagaatttcaaaccaagtagtttgactccagggcctatgagtttatacattcatagggctgatattcaaatgagagaagagaagtaataaataaacatataatatgttgagtggtacagagtgctacaaagaaaatatgaagtgcagttggagatgaattgtcaaaaaaggtcttagcacttaaaaaacactaaaacagcaaacaattctctttaccacctaaactgtaagagcgatctggaattgctataaagtacacaacatgggagaagtcttaaacaacagtttttattatttataggcccattgcacactgtcattaaataccaatatgttcaatcaaccatgcattcattgattcaataaatactgtacatacaaaatagaaatacagaaatgggtaagacaagtccttgggcctaaggactttataacctggtatttcactcaactacatgatagcataaataatgtttgcttctgtttaagtattccttaaacattatagatctcccaaagaaaattaaatacaaacctctttttaaagtgaatttgacaaagcaaaataaattggaatatatagataaatatgctaaaatttgtcatatgtactttgcgtactttacatgtgttatttcattctcagggcaatctaagacagtcacttttattatctcattttatagagaagaaagctgtgcagtaaagaaatcaaatacctttcccaaggttacagagctagtagtagagcctggatttgaatctgggttctgactgatttttaactgccatgacaaggatcaaagctcaaagtgtgatctctgtgttagaaacatcggggttgctctttaaaaaagccgattctcaggcctcaacccagacctactgacccagacactgcaagtagaatccatcaaaatgcagtagttactttgagaatcatgaaactctgctacacagtctgtcttcctattcatggaagtcctctcctagtatataaatgtgaagtaatatttctatttcaaacctgtattgataactgtctggaagataattttcctgggaatatattattgatgagactgcaaaacagatgtgaggtattggattgatctttccattgtagctagggaaatactgatgttcattgtttcagtgaagttcaatgatttcctatccgaattaactcccttaatttaacaattttttttttttttttgagagtgaatgcccctctgggcttctaggccacatggttgctagagaaattaggtactgtgttgcacttgaaaacactaaaatctttctgactactttcactgagcaaagagacataaaatgctttaaatttgcaacatttcagaaaataaattttagtgattatttatgactcgaatctttcagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcaggtaaagaaaaccttaaaaaattaattgctacatggaaattcactatctattcttttaattgtcaaactaactgtagtctataatagatgtattaaataaataaatatattttgcttctagtgtaaacctcctactgacatgtatcatttattttggaataaaacattgcatctgacactttaacaatatagtaaatcacttactttatgtgtatagttactagttggcttatcactgttgaaattatttaagaaaggtaaatagtggagattaatgtgtgtgtgtgtctgtgtttgtgtatgtgtgtgttcttaaacaacactgagagagtttattaagcaagttctgagaagatagtgagttttcaacagaattttaaaagcatttatggcatcacaatggatgcctatgttttagcctatactatggaaatttttcctactgctctaagcaactgggaaatttataaagtaatatgatgttgaaatgtgcaaattacattgattgatggatgcagccaattttaaaaataaatatacactttttttctaggacatgtatttttcaggatttatataagattacatttgtctatgcataactaattgtaataatttatgtattagtgcacagggattaccgaaaatatttcatgcatctacatctgagcatgcatttgaattggttattgaccactgaatttttggtgtaggaaaaatatgtagtgaaacaatgttacaaaaagattacaattgtttggaatgattaccttcattgactttaagcagtaaaatcatttgctcaacaaggttgggtgttttgtgaggctgtataaccatagtgtccttttgcctttagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagaggtaagtatctaatgaagacttattagatttttagagactattaatttagacttattaatttttagagaaattagggagatggcatatgaaaagtaatatgccattttctcagagtttacttgtttggaaggcagctgaagaattagaaaataagctcataaaaccttggagtaggcaatctaaagacacacaagcacatataacctcatctaatttgtcaggaagaaaattccttaggtgctcactcagatcttgactgtgattacattgtagggactgtaattatctcttttctgttgcacagccactaagacatttacaaaaaaagagcaaatccggtgtttataatgctaactctttcttctaaaataaatagagacattttggtactccaaagggaaaatatcattttggggattaaaattagctttacacaggtgttactggtttccaaaataaaccttaccttgattggaattaatcaacatataggtagttacattgcattaaaaagttcagaaagttttgcgtttagcatgatcaaaaacttctttttaaaaattatgaggatttatttatgattttctttcttcatctgtcgagcatattaaactgcttaacagcatcaacctgaaatggatcttaatgtgcaggggatttaactctttttattgtaaagttgtggataaaatatttaatagatatggatgaggactcatatcagtaacaacccaatactttatttcaaaatgaatagatctgtattacaatcacttgtgttgtgtgcagtagattttttccctttaacttaggaagcagttaataattaatggctccattttttagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggtaaggaaatcaatttgaatgttttcaattgcaacactaaagaaatttaaacttaaaaaaaaaaaaaactttaccttaaagctttgcgacagtatgaggtttagacaaggtgttgagctctgttttgaatcatgtaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggtatgttttgatacaacttacaaatgcttttaagtgatccttcaatacttatgaagtgacttttaataaatgtaaatattcttatccataagggatgagttgaaaaatagtatattcaattatagggacagttcagaaaactgaattatatttattaccaataaaatcttgtattctagattcagaaaatgttgatttgagggtttgaatgctggcttattgagcaacataacctcatctgtgaaaccggaataccaaccacatctatctcatagaactgttataaagattcaaatagacaatacatggacctaatttaccaacatgtctgccatataataacagctgcagcttcatgaatgtggcaaaagcagagagtagataactttctagtcagatgtctggtagtctgcagcagttcagaattctacaagtgaacgtaggaataagtttttaaaattccaagtagatagatactaagtgaatctttaaaatgttctcaaatttcctagagaaatataggattggttagaaagggagggattagaaattatagaaaatattccattattttttcacatcaaaaccacaaatttatgtatctccttaaatgttgtttttatttaaaaaatgttttattacttctcaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtaattctttttgttaatttgagaataaaaattaggatgtaattttctccttataatttagaaaatagatttcataattatattgtcatagattttactgtcttcatatatttgttataatttttgtatttggaatgatatattttaaaggaatataatattacagatctggaatttgttttgcacataatcatgtagactaggatcaagatgaggatgagattatcatggaagcagaaatatttatgaaatatatctttgtatttgccttaattgccagggatatgggaggcaaataagacagttttcaggtgagttaagtgaagcagccatattttataaaatgacagaataggtaaaggaagcacacctcagtgtagccatagcaggggttttatgactcagtgtgacaatgctgaattctcatagaaatattcattaaaagccttgaaattaaagtcaaaagtgttacatggtgacatactcaaatacttttttttttttttttgatatgctgaacaatttacatttcttggttccgtgaattcaatcagtgattttcagtagagtatgatggaaatcattgaattcatgtagcatgtttaggtgctcattgagaaaaggtgaagtcatggtaaccatgtttcaatattctcatttgtatcttgacttcctgcacatggatttttgggcctaaaagatgtttttaaaacatgctcatacacttcagaagatgaaaagtgtatgcattataactactttgggaaagaaacagtcaacatatgttactgtatgtcattctgtagattacatgtgtggtttctcatgtctctcagaataaaagctaatgtctttacaagacctgcgatgctgtgatctgtctggctcctcggttatcatttttaaaaaaagatatactttgtacaaatttttttaattgacaagtaaaaattgtatatatttatggtgtacaacatgatgttttgatatatgtatatgttgtggaatggagaagtttagctatttaacatatacattatctcaaatatttatgtggtgagaactattaaaatctactctcatagcaatttacaagtatacagtatgttattattaactgtaggctgacatactcaagttttaaacattcctgagagtcattgggacaactatgaaatgcattagattgatttaatataaagcatttgaagacaattttgaccttactttgtttagtttttgttgttgttgtgtgtatacatttaattttaatcaaattaccccagaaataatgcctaagatctgtcagtcaggacataatattattagcaaaaagttgtccaaaatttgagacatgatatttaaagctaaataaactcctttatacccctcttattggcattgattgggaagtttaggttgaatttaaatgctttggaactcaggaagttaatgtattagtaatagtgggttaacataaaatgctgaattgtccttgctgaatcctacatcttaaccccagacttcaaggtatacaggaaagtaccagacatggtgcatccttcctctgaagaaatcccaaactgtcagacacagatccctaaaatatttctttttcctgcattaaaatgtgtttcagatgaatggacacgttttgagtagtgtatgtggaaacgtcatttacaaagtctgtttagttggccaggtgtagtagctcactcctgtaatcccagcactttgggaggccgaggtgggtgtatcacgaggtcaggagttgaagaccagcctgaccaagatggtgaaacctcatctctactaaaaatacaaaaaaattaactgggtgtggtggtgggcatctgtaatctcagctactcgggaggctgaggcagagaattgcttgaacctgggaggcggaggttgcagtgagccgaggttgtgccactgcactccagcctaggcgacagagcgtctcaaaacaaaacaaaacaaaaaacaaaaaagcaaagtctgtttagctacccatataggaaaatgtttgtgattactctcccttctctagacccatgtcccataaatccataaatcccatgttcatttacagaaagcagtctagataggagtttctcagtctttgagctgttgccattttggcttggataactaactctttcttatcgagggtcatcctgtgcactgcagaatgtttggcagcatctctgtctatccactagatgtcagtagtatctccccttccctcagatgtgacaatcaaaaatgtctccggatgttgccaaagataaggggtggggttgaataccagtgatttaaacaaattaggtgtatccttctaaaaacattttacaggtagcgactccagcatctttatattagagtaatctggagaaggttatgcctctctcaattttccctctttccatttttatttgtagggcagcaatgcattcaggcttttggtaactctttttcccaagatagcagtaactattatgcagtgagtaatacgacccaccttaatagatatgaatagacttgttttgtgaatatattttaaaatataaatgtatgggattctgttcatgcgtctgagaagccacagggtacatttcctctttgtggagctatttatttttctggagagccaagacaggtatttccacttcagtggtgtgatttgaggggttaggaaaatttccttgccttcaattttctttccaacctagatgtcacaaatacataatagtagtccttaactttatttttgttttcagtcacctgaaagacatgacaatccatactccatattaatgcagcggcgattctcaaatagagaagggctttaaaaaattagaaatctctgccgggcgcagtggctcatgcctgtaatctcaacactttgggaggccgagatgggcggatcatgaggtcaggagatcgagaccatcctggctaacacggtgaaaccccatctctactaaaaatacaaaaaattagccaggcgtggtggtgtgggcggctgtagtcccagctactcgggaggctgagtcaggaaaatggcatgaacctgggaggcggagcttgcagtgagccgagatcgcgccactgcactccagcccgggcgacagagcgagactctgtctcaaaaaaaaaaagaaaaaaaaaaagaaaaaaaaaacaactagaagtccctactccaacttgaaatttggatgtatctccctagagtatgtttcttctctatgctgcattgcaatttttctttgttgttgatagttgtccagattgaggggaggcagaacaagatgcatctatatgtttccatctctccgaccgattctctcccttccccctctacttgctttctttctcttttccctcttctgtttacccgattctatttctgattccagtatgtaacagttccctctgaagctctctcaataccaacaatcctaactaatggtttttaaaagtcaaatattaagtactggagggatagaatgagagaataccaagactgataagatgcaaataatacttttaacatatttacaatctaatagaaatacaagacatgctcaaataagttaattattttaatatactctctctgagcataaaatataattatatatgctcattatagacatataaaaaataaataggtagaggctttccatagatgtgtaatttcaccacttgaaaattactatatttccttatagactgttttgtgtgtattcacttatatccatcaagtgactacatttcaaggcactatatgagaaccataaatattgtacaaacaggatttgctaaatgtcggtggagagtaacagtccacggggctgatcatggtcagtttgtgaggcaggcctccaaactccttggggattgagatgatggagtagcagagctcttcaagggtatggaggcctgaaggtacaaagcatgctcaggaaattttggctattgcggtttgtctagagcacttgttctcaaccttacctgctcattactaattctactaagtacagaattaaaagaagaaaaaaatctaatgaccatttcctcctgggactaattagatcaaaatctttgaacccagacattagcgttttaaaaagctcctcagatgtactattcagccaggactggggcagggaaagctactgaactccagccttgagaatgagaagtagaacaagaggagaactttaaaaggatttaggggccactatatgactatggagctgaatttagatttgatttagtaggcaacgcggaataatttgtttctgaacaggagagtgacacaatcaaagtggaatgataggaaaattaattttgcaagagagagagaatgagttggaagtaaggaactcagaaggcctcctgggactcagcagaaagctctgaggccaccaaatgggtgtggtggtagtggaaatggagaagaagggaatgtaaatgaggctacacagtggactgccactgttagccgtggggttagaccacagcaagagttaaaataattcttcaattttaactccagaagggcctcaaaaagactttttgtcttgttatcatcagctatatggaaggtagaataaaaactagttaggagaaaaggtaataaatgtggcttttgataggctgtgattgagttggaagggcataccagtgaaatcaccaacacaaagttggaagtgtaggaaagcacttaggaggtggctataagtgaaaatgtgaaaattctctacattaaagggatagatgaagtcacagaagtggatgacataattgagcagggtatgtgtagagggaagacgggaaggttaaggacaaaatctttacatatatctttcttggagtagaaggaagaggaaatgttaaaggagatttgattcaatgaaacaagtaggtcaggtttctattcaaatttacaacagatataattacaacagatataatttatttagtttttttcgcttggacagcttaatttaagtgctttgtattttcttttcaaaaggtgatggcagttttggatcagtttaccgagcagcctatgaaggagaagaagtggctgtgaagatttttaataaacatacatcactcaggctgttaagacaagtaagaaattcaataatataattatattaaattgcacattattaatctactggaactcttattttgcatacagttgtgaaaatgcaaaataatgaccacatttctacttaagtttaattatgcaatcctagtttgtcttttcgttgtggagtagaaagttttgtgttatttctcctgttgagaaacaaaacactgtatctgagaatccttataatcgtgatacatagtgtgttgtaaaactttttgtaagactcacttacactcctctttttactttagaaccttgctgttcaaaatgtgctccatggacaagcagccaggcattacctaggagattgttagaaatgtagaaacttgggacttttcagtgccatattattgttcctgatactccacagtagtcagactcctagctgcctccacctgcttccagaccttgaagcctagcaagctcctgacttcgccttctgttttcttcagagtatttatcttttacttttctggtctagggagagaatgatttttatttttattgaacatgacttctgtgtgttcagggtgaaagaagaagtttaatgcatgatctcacattgctaatttgattgaaggttagaaatcttaaactaaaactctcactgataagcttgcacctctcttttctggatttatccactttaataagaactgctattgattacttgctacaaagatggagaaagttagcatgcttatcctatttcctactccctgtccctgtccacttcctaaaacttaaaattggttgcattaattttcctgatatagtaacaattataacttggaatgattttcaaaacttttgtttttttagtataccaactctagacagcatggactgactccttgctatgtgagatgaggaaaattaacgctattctttctccttttcccatcaccttctcaagttctttaatttattctattatttttatgtagtgaaagtttataacatttatattctggtctgtactcataattaaattgttcacattttgtctatagtttggttctgagaacaaaaccaataaatgccatttatatatttttttatttgtacagaaccaaaatatttctacttctagataaagaaatgcaaccttctgtcactaacttcttttactaatagaatagtaacattccaaatatcaaagtcaaatggattctctattgttatgtatttatcatcaatttataaaaataaaggcatattttaatttggtcacatttttaccctgatttaaaaaaaaatttgtttttagagatggagtctcattaggttgaccaggctggtctggaactcctggcctcaagtgatcctctcaccttaaccttctgagtaccagtggtgatttattttatgtagctttttgaggttttctgattatatacatatatttttaaaaaacgtacttcaggaaaagatatatattttcatcatgacttcaagtgtttctaagttcttaatcatacagtttgtataacagaatctactttcttcttgaagacattcctcattcagcacatgacttactgctctaaacaggagagatggatttctaggctgcttgtgcagtgattaatctatgagttagtttcctcgccctctttgattactctcaatatttcttggattccatccattctcttggttggattgtccttagtttttgttgaagaatatcttcgagtaatttttttaagaaaaggtgtttgtgaggtaaatgttttcagtccttacatgttaaaaatatcttagttttgccctcccatgtggtggatatgtcatcacactttattttttaggaatctaggcttgaaacaattttcttcaaaatttgaagaaaattccattgatttttagtgcccactgttgctaatgaaaagtctgcagtcagtcagatgtttgctcctatctaggatacctttaatttcattttgaaaactgaaaatttgaccttttgaatttcatttgttttcagtgttctgaacctttacaagtatgtgtttgtgtgtaggttgtttttcattccatctaattcattattttgtgaaaaattgtcttctgtgtattctcttctattatttattatttcctccctaacatttattaatcatttttattgacaactactatgtaccaggttaggtgatgggacatatgatatatatatagtagtaagctaaacccagtcaaggctctgcttctctggagcctatatctagttacttatgattcattattgcttatcattgctccaagagtatatgttagatgacaagccttttgggtctatcatccatgtttgagttccctcttcaagttttatctataatttgtgttacttacttgactgtctcttacaggtttctaatattttttagaattgcatcatctattatttagctttctggtgaattttgttttgataatcatattttccatttccagcaattctttccatcccctctggttgttcctttgtagccatgtttttggataaaatgtccataggtgtttctgttcatgtcaattagaatttttttttgtattacttgcattattgcttttttctctgaggttatttgctctgtgggttcatcttgatctttctcttttatcttgtcagttttccaaattgagtagttttgggtgacttcgtatgaagtaagtactctattgattgttaaagaaggactgtattgattattaaaggtaactagaatgggcattcttcacattcatgtaggtttgcttgttcaagttaccactttctgaacaagaaggttagaccatagacttttaagggctgcatactgcaaagggatactctgttctttaggttacatgggcagggatcactgctgagaccatacctgccaaaggaaggcaggctttgctctctagatgctggacttgaaattgtttcccctctgcttagtgctgcattattttttttgcttcttaatctgctgcagagtatctagatcagggtgtccaatcttttggcttccctgggccgtattggaagaagaattgccttgggccatatataaaatacactaacactaatgatagctgatgagcttaaaaaaattacaaaaaaaaaatctcataatgttttaagaaagtttatgaatttgggttgggccacattcaaagccatcctgggccacatgcgacctgtgggccacaagttggacaagcttgttctagatacttcagactctgttctacatctcttcatagatcaataacttgcagcaatgagtttatcagataaattatgttcacttttcatccataaaaaaagtcatgggaggtactcaccataggattggtttaatccagtcactctggccaattttctttaaaattcctttaaccttggtatattggtttaattccttcagaatggcttttcccattgtgataaactggttccaggcttcacgagtacttattccaagtacagaaggaaagagggtacctcttttgagtacttgtatttcaaaggaatcaaatggcctacataataacccattaaataaatagatgtctctcagccccagtgagtcatctgtgcttttctcaacaagcactatgttcagggcaatgctttgtgctaattggctttaggcctttgttaccaaactgatcactgtggccctgagggtgctgttacttagaatattcccaccttttctgagaggctgatgttggggtcacttccccctgaactaaagtccaggggctgcatgggtgagggttgagtaactcagtactctaaggaggaaaggaaggggaatatacactgttagttaacagtggttattcctgtattctctcttgtttggattctactggggatttctttctttttttgagtctttattgacattaggaatgagagatagaaacagggtgagagaggaagtaaaattaaatgtgaattcttccatcttataccagaactcaactgtatttttggaatctatatatccttacttttcccttgatttattacaaaaaattcttagtggctacactaagtaaattttgtaacctttaaaaaatacatagttataatattttaagtactctgagtaatagaagatttcatgtgagtacaaaatatcctgggggcattttaattattagtaacaacagtcacactgtagttctagaaccaaattgaacattttatatatagaagcttatctgtgaatcaaatctgacataatctcttttaatgtgtaggtaatttcttatataaattgattttttggccttgctgtcagcatgcatttcaaattttacaccatgtgtttggggagctatggtggaaattagtacattaaaaatgtctacagagccaattagtgtataagcagggaggcaaactaagggaatggtggaagaaattaaaaaaaaaggggtgtgcataatgtgcttcttctcttaaaaaaaaaaaaagaaaaagacaacaacaaaaagaaagaaaaatgacatttgacggtcattctaccagtagtcatggggcagatctatattatactgtactattacgctgttttttctttgcaattagtgagttgcttttccaggatagaaaatttggattagacctctgtctgtgcctatgaaaacaagcagagctaacttaactccttctcatcagttctaaccaactgacatgggcattaaaaaaaagattttatctactaagcaaatatgatcacagtctgaagctttgttcttggaaaatcccctctcagggtgttcagcctttcttcttcagcttgcagaattctccatgtttcagtttcctgataaatcagtgggcgccgctactccacatctttgaagctggttgttaagaagcagtgcttctgcagcaatcacagtttaaagcatgaatcaatttaacatcacacaagctatacatttcaacagagttacagtttcagagtaaagtgcaatatacagtataaagcgaatctggaattcaagcccaacaatgtcataaaagaggctgtgaagtctcacatgatgtgggccacagagagggttgttgccattggatcttagctcaaatactaccacatcttcggagtggcctgccatgggccacccttactaaggcaatccctctgtccaagccaaaatgatatctcggactttttttgaattgcagagagattgttgcagccagggattgccttagtaagggtggcccatggcgggcctctctgaggatgtggtaatatttgagataagatctgaatggcaagacttcagccatgtgaagggtggagggatttctagggaaaggggtcagcacaggcaaagaccctatgatgggaagaagctgggcacagcttgggattgaatgaatgccaatgtggctgaagggtggtgattgaagaggagggggagacgagaaggtctggaaggcctggggcatgatcagatcaggtgctgaggccgtggaagtagatgggattttcatctaagggaaatgggaagtcattagagagttttcaggaggggatgatatgtatttttttaaaattgagcattatcctcggtaaacttttgtagtcgttaaaccagagattataagcaggttttacctcatatgccagttgcagctgattagtagtggctatagagaatcctgggctgagaaggatactgtggctaaccagaattcagtagatgagtttgacgtggcctgttagtatgactacactgtgtgcactgtttctgcattaaatgtctgataaaaacagagccaaaggaaaaatagaacttaaaaatttaattctgacagtacagttgacccttgaaaaacataaaggttggggtgctgaccccttgtgcagtcacaaatttgcatataacttttgacctccccaaaaaactgaaccactgatagcccactgttgactggaagctttactgataacataaacacttgattaacacatgttttatatgttatctgtattatatactccattcttaccataaagcaagctagagaaaagagtattttatttaaaaaatcataatgaagagaaatatatttaatcttcgttaagtagaagtggatcattataaaggtcttcaccctcatcatcttcacattgagtgggctgaggagggggaggtagaggaaaggttggtcttgctgtctcaagagtggcagaggtggaagaaaatttatgtatatgtggactcatgcacttcaatcccatgttgtccataggtcaactgtagtttcaaaaccagctttttattactgaaaatacgggaaaaaaaactcagagaagaaatggaaagtttgctatgatccagtcatacagagaaatccatgttcagcctgttgatgcactttaaagaaggagatacgtgggtaaaacctgatgttgaattactcttacatgattttggacttttgcaggagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgaggtaagtaggtcatgttgttttctattcagtgcatgacaagtgtgatccagacttgctctcaggttctgagaacacttcccagtaacactgtgccccagtaacaatttataaacaatttggatgaaaactaccatttccctgatcaaattttgtaatttcagaaaataagagtatggaaaccatgcagaacctcatagcaagtagtaatagactttgaacccacaagttctgctctagaacccatcatcttaaccctgtactgatctgccttctataaaaatgtataagttaggcttcacagtatcaaagtaagtgtcaattacatgattccaatgaggaaagatgagtccatacttctcaaggggactagagtgattcatgttggattcttcggcatgaccatctcacatgtctcagaggcacacctaaccctgcatccagagcaagctttggagagggagcacactggagtggaaaggctgtggtctttgaagacaaaaggcctgggattcatcactattccacacatttagtaactgtgattttatatctctgattcccattttttaaatagtctgtgaaccatgactaatatttaatgcataaaattatgatgacttctgtaataattggagacattccagatgaaactcttgatgtcccctctgccattgctccccaaccccagtcaccctgttacacctgagagtcaccttacattcctttcttcctctctcatttcacagctaatccttcagcaaatcttttcagctctgccaccaaaatatatcttaatgcttctaacaatttctctcactaacgtctaaatctgagccagtatcatctctcattgcctactggtcccctgcttctacctctgtctcatgatagtcccattcctcacccagcctctggagtgatttttctaacatgaaagttggatcaggacttgttcctgttattacccctcccctgccttatttcttgggtacagtgctcagccactcccatccctgaggttccttgcagataccagaggctttatatctgctgttgatttcactcaggaatgtctgactcccagatgtgctctctacttattataaaggattatctgaatctttctgaatcctttcatttaggactctcagcagagaggatgtccgcaacgaccctttgtctctccagcccctataggactattgctgcctaggattctttatgttttcattttttaaaaacttatttattgtctgtcttgccatcagaatctaagtaccatgaaagaagggacttttcgtcttgtttgccattgtatctctagctcctaaaatagtaagccttcagaattactgtgttgacagtaggggaagggggagaaaggaggaaagaaggaaaacagtgcctggggcatagaagccaagcagtgtatgcaactttccttctcttctttctcttctgaaatgctatgaatatgccttttaggtagtatccagaaatgttccttcctgaaagggtccagaaactactgaaaactgtacagattatgaaatgaaacagggtgcagggatttggatttgagttgatgtttctgcttttgaacaccagggggaatcttgggttacattaatctaggtaaagtgcagaatagtctcctgtatttcagtgccctctttccttcatttaactaactctaggttctagtttttccctaattcttccacaaatccccaaagtgtttatttataaagtgaagaattgctattttttaacactgttcgaaacaccttatctctaaaatgacttattctagttctctgaaaccttactttaaataacaaatccagcagtttctgatgaagtaaatgaaatgtcagcatattttaaaataatttgcctaatttgttcttagcataatgccagaaaagctttctggattttgtatcacaaaaggctagtagatttcagtagctatcaatcttctaccagcactaagtatattttaaaaactcagcattaaggtttatttttccaagtatgtttcagcacaggaaataaaatcatgctcctttggagtcccttaaatgctggagctgtttagagtgacatacaagaactttcttcacgttacatgctctctcttcctccatcttgcttttaactgttagcttacttctccaattcaatccacttcgtttgaactctttatcataattctataaaacttatgaaaatacagtcaactgcattttctgtatgtttctgtgtttcaatatcttcaaaatggaatgtactgccttggtacatcacccactatgaatctgttatttctgttatatcccacagttgccaggccaggatacttgtcccatccaggccaaacaccttcccccgaaagcaagtatgcatttgtccaccaggtccttgactctattttacattatctttttagtcaattcatttatttttatgccactcctgctgtcttggttcagtatgtccagggaattatcagaatttcttttctaaaataaaaatctgtttatgcttgcaattccttgacagttctcaattatctgcaaagtgcatccaaacttcttggcatagcatcaaagatctttctgtatgcctcttgcttccctttgcggcccctgccaccccactgcccacactgcattctagccgtgatgacaggcttgaattttcagttatgctcatgtctgtccatcattgtatttgttattcctctctttccaccaagttgtctgcctagagagctcattttccttaagaatttcttcacaaaccatctctactatgaagctcaagtgtgtcatgaagtgttagcttctccaacttgtgtttcttgcagacactctgtgcaagacattgacttaggtgctaaagagggaaagctagatattatattgttcttgaggttgaaagcttacagtctagtaggagagtcaactttgctgtctttacctcagtgtttttctccctctgtgcttccctagcacgtggtacttacatatttctggaatcttgattaaacacctgtttgaggactgtctgagcacaatccttctggattgtgacaccctcaagggagcagagatacaaagatggctttgtatactaaatgactggccctcatagatacctagtacatatttgtcaaataaatgaatgcattctatttttggaataattctattcagaatcagataaagtttactttaagctatgaagaaagaagtctcttagcaactcttacaataatcacaatcaaagaatgactgtttaacttaatataaaccagtttgttttaataaaatatttgacaatagtcatggttacacaatgcataaattatggctaaattattatcaggaaggaaaaatctttacttattatttcaaaagctattttgctagtctattaaaagctattagaactgcacttcttaagattaaattctataattgaacattttaactaaccaagatattatctctttgccactgacattatttcaaattaagcttaactatttctttttagcctttggaaagtattctgaaagagtctgtgttctataaatatacttaaagaggcatgtcttataaaggatttggatactattcaatgatgtatgacttggctttagcttttttattcttaatctctcagcttttctcttcagcaggggaagagtacctaatggcctttcagtaatcccttggtaaatttttctttcaagcccattacttactgtgaaggtcaacttcattagtgtatttatcttatttttttcagcccaaaataggtatattgaaatgaatgggcctaatgtcaaatgtcccgactacatcctggaagagagagaatcttcagctgtattagttgatgcagttaaataatatgtactctccaggccctcatacaattgaaagttcagggtatcgttgctgctctgcttctaatccttccagaagtgattggtgctaggtgatggagtaactattaattgatataatgtgagccaaaaccaacagtcacgaataagcaaaggatttaaatttaactccattaagtcttgtgagaaattattttcaacataggttataacatacctgtgacatcacatgaaatgctgtagtcaatttgacatcatggggcagagaagacagagttggaaatcagaattttatagacatctaatgtgataataacattagtagctgagatgcggtaagctctttgaccatgtttccagaatggataagacctggttgagatgaaaactttacactgtttttttatattaactatcttttactctttgcctgaaatgtccaactctagttgctcgtgattgcgtgggtcagtctccagaaggttggactttaatattacccgtcatcttttccaagacaaaattgtattcattctaactcttagccccaaattttcttttttaaccttaatatctaacatgattaggtttatggtaaattatatactcaaacagaagaagagactaatagcaagcaaaagtcttatattttcatttgttttcatccaaaaagtagaaaatattttccaaacattgggaaacattttagtcagaaaaataaatatcaatgataaatagaatagagaaaaattttaaagctgagctaaacctctatgtggttttaggaaaatcaaaactattaaataaatggcaagtacaacaaaatcccatcaattcttatttaacatacttacattttgaaatagttaaaatattcatatgatcattgagagaattcagaattgcctttaagtaattgttcacatatacaaaagaaaagtctccaaaaattgggtctttgcctgagatagatttgtcttaaaattgaaatcattcacttatcagatttgacccttttttaaagcataactttgctgtgtaatattagacttatatgttttgatttccttctacaatatctcttaactttaagggacaaagtgagcacagaatttttgatgcttgacatagtggacatttatatttaaggaaattaggacaaaaattattataatgtaatcacatttgaataagatttcctgtgcattttctggcagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccaggtaggtgatcaggtctgtctcataattctatcttcaggatggataaccactgacctcagatgtgagttcagaagagtcaaaaggaaaacagagtctatcacattgtgaacagaggtttattttgtgaaaaaatgcaagcatcacattgtgatttttatcattgtattttgtaggaaaaaaacaattgatgtaatttttcagggcaaaaactgaataaaaagaagagaatgtttgatatcaagttatatgttttaaagttagatttgtagattctttagatactctagaggtcataaaaagtaacagcaaaaactttagtctaggtattgttggcacttgtgaggcaaatcaaattcaggtccacaaattctttttcataattctgaaacccaaagaactctgaaaatcccaagattttttaaaaaatgactaatttggtgtcaaaacctaagcaagctgacttgttgcttattacaatctttatttctcatgctcagtgtgaatatgcatacattttgctgcagaaatatatacatgtttgagtacagggggctggccgtgaccctactgagggtttctgtacacatcactgtctaccctgtggaatcttacctccctttcttagttcccaatcctgaaaagcagttatggggccagtgctctgtacagacatgttgtctcagacatcagtttgagcaggaagtaaatcatttaggggttggcatttgtttggagtgtggggaacactctatctttagggaaactttatatagttagttatttgtaagtaaaattacaggtggctatacatcatcttgctgattgcaactcaattaaatcaccgtgcctggcacagaagaaaatatgctacaggatatctcactagggaaaaggttctagttcgtttcctgcgcactcaacttttgtacttagataagcaaatggccccagattccaatgcctggttttatttttgctccaaatacatatatactcttttgttttggatagttacattttagaagtagactgtgtattctcataaacacttcaaagtgtatgttctggctgagagtgtctctgtgttgttcaataataataagactaattatcattttttgagtacctgctgtgcgtcaggcccagtgccacgtatattagagacaagatctcttatcctcatgccagggctggaagttagctattagtttctcatttgccaaatgagaaaactgaggctcagggagattatgtaacttgcagaatatcactcagtaattggccaagataagaattcagtctaaatgagaaccagatccagagatatttggctttaaattctatagtctctcctaaaccatatgcaactctaacatgaagaagcttatttaatcttcactattaaaaaagtcaaaacaaaacaacagagccatgaatagcaaatattgtcaatgagaggtttggaaaaacagtcttaaaggatgaaattccatagacctgatatatttccacctggaaaaagtgggcatgggacagtgattttctcttgaaagatctgctcatttttgtcatgggacatgaaggtggactggaccactcagtttcttctttctgcatctcccaacccagtctttctgttcatggggtgaaaatctgttgttgaagccttgtctgcttaattggacagtggatctctcgggtccctgtgggctgtgcgcttgtacttgagctctgcttcttcactctgtggtctaggccagctagcagccagctgagttcaccttggttcagactcatggcctttcattttcagtatctgacttcctggttttgctgaaaacctgtctaaaatgtaatatccatctgattcttcataccaagccacacaattcttcctgatcccttttaatctccaatattgaatggtggtaacataaatatggagacagatcatgtcagaaacccagggcctaatcttttcttttctgcctactcttctcacaggctgcttagtactttgtaagctttttttttttttctggctgtaacctagattttctctttatcattactctatttattattgttagagcacttctgattatctcagccctaaactctgcctccaattttaaataacaataactcccactcctgctaatactgctactactactaccatcaccaaactttttcttccccaaagcagttctgtttgggaaggaaacagttccctctcatacaatttcagttatcttcttgtcttttccgtttaatgaatcttcctgttaatgttacatcttttaacatggaaacttctagagaaacaaaagacgatggatttgttaaaccttttgggtgtatttttatactaactcttactgcagcgtgtgcattatgagtgtaggtccattacggctgtattaggagcagaaccttccagagcatgagcgatgtgctgggcttgtgcttagctctatccatgagttaagtatctcaatccttaggaccctctgacatatgtgctattattatttctagtctatagatacagagactaaagtttagagaatataaaaaaacatttacaaggtcctatgggacaaaaactgtaggacaaaatgcaaacccaagcagcctgagagcagagctcctggtccagcactgtgatagctggggacgcagagacagaaacaatgcaattattgacagggaccatggtgctgtgtctgtccacattttgaagataattatggtttggatattttcacctttaaataacttggagagtttcaacattaactcagtcagatggatacatttatatcatatcctgctgggagtgacagttaattctgggtcctatggcaattgcacttttgactgagatgaatgctgactgatggctttaacatttaactaatgcgatagtatttaacacacccatataaatactatagtcttcgggtaaataaaatgttaccggctggacatacatgaatatctgatggagattatggaacatactctactcatacttctctgaaagtaaaaaataaaagatatgtttcagtacacaatgtgatatgtactcagacttaattcataaatttctcttatccttcatccgtggatcttttctttatttacttattgcgtttgttaaaatgcaggcttctctgaaaaattatttttaaaaatagtttttagacaatgaatcatattttctcaagtattttaacattgtaatcattatgataattatccaaggggaaattatacttattttttattcatttattcattcatttggcaacaatacattgaacatttactaagcatcaaactggctctaccacttaatagtggcatgatgttcatcaagaaattgttagtgcaatcaagaacactagaaattcattggatgaatttaaagaagcttttagaagggtattatattataattgaggcactttatgaatatataaataatattatgttttcatgctagagatcatgccaatgaagatatttactttgaaaaggagaagattagaagtttaaaagcatttccatattgaagtaaatattcatttccatatcttcacagttatctttctctgagttctctgactcattgtgaaaaaaaatcccaaccttcttcacagctctaccatcttcggattgttgcctagaggggtaaaaactattgtaaaaggatgtgtgcactggatgagaatttagaattagacgaaatgacccctagagtcttttcctgctttaagagcctgtgattccaaattctaacagtacatttatcaagaaaaaatatgctgaacatttaaatagtttttgaatagtacctagatataatagatacctaataaatgtgctcaatgaataataaataactggttaagatttaaataagcctccaaaatctcttccacattctaagaagggaagcataaaggttgttaatgaactagtgactgtgtgggtagctcattatttttaagtactcttgactttgctgttcattatctgtgtggccttaggaaaatacacacatttctgaaaggattatgtcgtttgtaaaatagaaagtccttatctgtctaccacagatgattcttatgcaaatcaaatgaaatgttcaataaggtgtctgtaaaatagtagagagagatgaattaggagctattgtgatttgtttacattatgtcacaggtgcactttattagggatatgttttatcttaattacacaattctttaacttagattttgagaattatattatggttatataggaaaatgcccttattctaaggaaatgtataatatatttaggtctgaaacattgtatctgtaacaatatagtatgtaaattatgctaattcacatgataattatatgtaattatattaatatattactatgtatacaatatatttacatgcatatatgtggggaaatgttatcagttagtgtagtaggggttatcatactcaaattcgatgtctccatccttccaactcttcatgcttttccagcatggtgaggactgctgagctccatcttttgctggtagtctctctgtcaaatagaactgtttccaaattcagtcatttgctccttgaaggctatgaattcatacttcgttatatttttctggctgcatatttaaattactttaacaatcatataagttcattgtaaaaattttggaaataaaaaggaagataaaatgcacagataattttagcaaatgaaataataattatattgggatgtatttcttcctagattttaattatgtacattcccatcaactttttattttgaaaatgtttaagcctaaagaacagttgaaagagtagtacaggctgggtgcagtggctcctgcctgtaatcccaacactttgggaggccgaggtgggtggatcacttgaggtcaggagttcaagaccagcctgaccaacatggtgaaaccctgtcactactaaaaatacaaaaatcagctgggcacggtggtgggcacctgtaatcccagctactcgggaggctgaggcaggagaatcgcttgaacccaggaggcagaggttgcagtaagccaagatcacacccctgcactccagcctgggcaagagagtgagactccatctcaaaaaaaaaaaaaaaaaaaaaaagaaagaaagagtagtacaatatacattcatactcatatacccgacgcataaattcattgattatttactttttgccctacttgttctttcttgctctctttgcgtatgaatgaatcattgaaattaagttgtagacatcatgccatatcacctctgaacagtgtgtatatttctttagaataaggatgtttacttacataatcataataccattatcacagctaagaaaattaattcagttgattttttccacatatttgataactttctgtctatccacgattatgtcttacatattcttttaatttatgtcatagcatatcatcttagaaagtgatccctaagttactgcatggtatacattgtttaaccatttccctttgtgattggatgtctttaggttgattatatttttattattatcacaaatgttgaaatcactctttttttctgaagaatttaaaagtaatttatctgtcttatggaataaaatatttatttccccttaaaagaatttcaggcatgaacccaagagagaaggcttttttttttgttttagttgttgtttttatttttattttttattttttgggtagaaggagcagagagacaagttcaggaaataatgagagtgttagaattttgttcaggttaaagtgagttggagtgaagtttagaaatctcctttctactcatctctcctgtttttaaaacactgtcctggaaatagttaatattaggaacgagaaaaatggtataggttttcctagtacactttatttcttaattatgaaattctacttaataacttaccattgaatgtttatccttattatcattcaaggtaattttattgaagattgaagatatttataataaagattgaaggattttattgtcctgtgtggtcaaccttggggggtgagatgttatgagacaggacaattaattgacttgatcaaggtaccttgttataaaaataacacagcctggtttagaacatctcttcctgactctcttatttggcatatagcctaagtgtatgcctccttggatgtatgagccctgatgttggtcatatttattattttatctgcttactttcagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctggtaagttctgttttctctacaatgaagattttttttcttaatatcagcagcttcatttttatttaattgtagttgtatgcttaattccttaaacagatgatcattttttttgtttagtgcataaatattcttaaatcttgtgatatattaataaaaatcacctgaaaaaggtagcagttttaggctttttaaaaaatccgcaattaatattggtgtagttaatattatatttagaaacatagagaaggaaattgctgttagaactccacatttggtgatttttaattttcataaagaattactgtgtactcattatcctggaatgttttcgttttcttggagtgaaataatttacatgcaggaatggaagactgaatgatctataataataatttttcataagaatcggtaaatgtgtatttaatgttatcaaagctcatttggaatggttgtctcatgctttcaagaaattagaggactttgtaattcattccttaaccattactttagttctcaccacaaaataacattttaagtttatttagctctttctcatattttctgctttccctttcatttaaaaaatacttttgagtgtacacaatgtgccatgtacaggaaatagagctttatcttttttgggtataacttcaagatcatggcaaaagaaaacttattattaattggataaaccttagatataatctaggttatttcccttattttactagttttctagtgaaaatattcaggtctctgctgggtacagtggcttacgcctgtaatcccagcactttgggaggcccaggcaggcagatcacttgaggccaggagctggagaccagcctggccaacatggtgaaaccctgtctctactaaaaatacaaaaagtagctgggcatggtggcatgtgcgtgtagtcccagctaccaaggaggctgaggcacgagaatagcttgaacctgggaggttgcagtgagccgagattgcgccactgcactccagcctaagcaacaaagtgagactccatcttaaaaaaaaaaattcagttctgtgttctgcatcaaccagaataagctacgcctcttataaaaaacaaatgtgcacaaaccatctgtgaggacataaggattaaatgcttgcttactttgagtattaaaataaaaagtagaagctttattatatgagtaaaagtgtttccaaagtctatttgaaatgcaggtacagaatgaaaatctgttattttattaaatcgttatttgggtctctttttattccataaaaaaaaaatcttttccacatctcttagtggagatcaagttaacaaaattagctttaattttgtgacaagtaaatttacataaatataggattatggagataatatttttctttgcaatgtctggaccttttataaacattgagaggaaatataaccattcttacttatttagtatgctagcatgatgttttttaatgttttagatccagttaaagaatatggttgtgccccatggcctatggttgagaaattaattaaacagtgtttgaaagaaaatcctcaagaaaggcctacttctgcccaggtattcttaaagttttgttaatattttgtacagaacatcatttgcatatatgcatatatatataatcttcaaatatatatacttaaacacataaacacacagagacagaattaaaaatagttataaggcaaacctcctataattttcaccatcccaggcacaaaaaaaggacattgccaaaacctcacatgctcccatatgcctgtctcctcttctcttctttgaaatgcctgcccattattttgcccatttttctgttgactcttttgcctacaaatcaaatcagaaaaaaatttttatatataacatactaatcctttcttggttttatatgttgcaatttctttttcgagctggtggcttgctttttaaatttcttgtagggtgcccgttaatgaatggaatttcttaattttaatatatataaatttaacaattcttctttttccttcttttcctggttattcctatttggtcctgttgtagagcccatctttcttttagagctacaaaaatatttacctgcaatttcttcaaacattttaaaagtttgcttttgacaagattttaatccattggaattgacatttttatctgtatcccattcctttttttaatgtggaagactaattttttcaggttgatttactaaatggcttctccccttttgtcccatagatctgatgtgtccattttgtaatttattaaagataatgtgcatatccacgtgtacactgtcttccactgatcaactcatttatttttcctccagtattaccctgtcttaattcctgtagctttataatgatctcctctccctatttatttttctcatccaggaatattttaaccagtcttaggcttagcttttctatataaaatttagaatcatgctgtcaaatcttatgaaaaaccacattgtcgtttggattggtcttggattgaatatgttgacaatctggagatgatatccttatgatattaagtctttgtattctttttcttatttatttatttatttagaggcagggtctcactctgtcacccaggctggagtgctggagtgcagtggcaggatcacagctcaatgcagctttgacctcctgggctcaagtgatcctgtcgcctcagtctccatcagccaattgtgtgccaccagacctggttaacttttcctttaatttttttgtagagaaagggtctcactctgttgcccaggctggtcttgaactcctggcctcaagcaatcctcccgcctaggcctcccaaagtgcatgagcaactgcacctggccaagtcttcctattctggaatggcatgaattctctctgtttacttaggtcttctcaagcgtctttcaataaagttttgtaatgctacatacaggtcttgcatatcttttgctggattttcttgctgcaaattgctttaaatgcagttcatggcttgagatttgttttttaactgcccagttaacgtaaatctggtctatgaattgtattgacaacaagcttgtgagtagcaatttttcagcaaccttttgctccctctccccgctccctgcacagtcccagaacaactttccttatataccaatgaaggtggaaatgtggacaggttgatttctccttcttgtttatgctgaatatgcagtcctttggggtcccagctttataggtacagtccctttactaagactgtctatcttgggaaggccctagactccaacttctctcccatgggccccacaaagcatccaagagtatacatttatattatctcactttgtcttggcagacaaatgtcttcagggcaagtctggctttaggggtccactgacctctctggtttctgtctctcactgtgatttcggcctgataattccttatggtgtcagatcttcaatgtttttaatacattgtttttaaaaaaatcatttagcattttaaattgtttttagttggaagatttgcccaagtaaccttgtccaccatattatctgaaagagaactcttgtcatgttttacactgatacacattttaataaatgtgggttatctttatgttgtgagctcttgatttggtattataattaattggaaaagttttaactttaagtattctgatcaaaatagtcaagtacaactataatgactttatcaaatattacataatttttcttctacttggtttacttgttttttaatttaggttaccatcaatgttagtcacatgaacttttatatttatgtccacagtaaaaatttttcatagcttgttgtttttctttcttgttttttcattttcaattatacttcacctatacttaacagaatacttaacaaatatgtatatatatgacaatattaaaaagcttagacatacttattttatgtttaaaatataacatatactaggcaagacaggaaaactcatcacttttatgaaatgaggcacagacagagtaatgggctttcttggtgtctcctgagtggcgggcaggtggccatgtcacagctctaatcccagtttcctgacttctggttctgttttcttacggtgccttcacactgtctctccagatcaaaaacagaatctagagatgacttctaaattttgttaccaaagactgaaattcctgttcctttcaactactagaagctcaactaaattgttggcccaagggtttttctctcactggcggtggctctgcaatatagaattgcatgcagagtacctcctgactccgctaaaatcctgtttaattgacccttgagattttcttttcaagttaaaaaaaatactacacataccaaagagtatcaagcacagtttaaaaatacatatttgtcttttcatgtaattttatttgtagtttagattactaatcttggtgatctagttgggttccagtttaacagttttaggttttgcttgacagagctataacacttcagtctatatttgatttttcaagggaaatgagttaactcgataagtactgttttgttatctttaaactttctcaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggtaaatccaaatgctctttaaatctttcataatttaaagcatataccatttggaaagttacttaggaataaattaaataagagccaatgtaggattattattcaattagccttctgttagaacaagagtattcaagagcaaatgtgttttgctttagaatcacagcatatgtcttagctcagggtccctagaaacagagcctgagatgggttatcttgcacaagtgatttaaggaaagagaatcttagagagaagtagaggaagccagaaagggcagaggaaaaagctcagcagaaatgtggtttctgaagaggtccagcctcagtctctgcccacaaggagctctggaatatgaataggagcacagaattttcccactgccaggcaagggagccagtctttcattctcacatattgttcagtcattggctgcaatctgctgggaggtgggggtagtgtaactttcaaggcatttctgggcaagctgcctcctgtcatctgagggtattctgtgataaatagcacatctctgaactatagtagccaacactcagggtagctggggatggcgtacctgatggataaaggagatctggacatggctcctaaaagtggatcaatacattgtgttggcaaatataagataagtttctagacttcaaagacaacctagtattttgactgctgcctgaagataaatattgtgcctcaacattagttctgaggttaaacaatcttttttttttaattgatcattcttgggtgtttctcgcagagggggatttggcagggtcataggacaataatggagggaaggtcagcagataaacaagtgaacaaaggtctctggttttcctaggcagaggaccctgcggccttccgtagtgtttgtgtccctgggtacttgagattagggagtagtgatgactcttaacgagcatgctgccttcaagcatctgtttaacaaagcacatcttgcaccgcccttaatccatttaaccctgagtggacacagcacatgtttcagagagcaccgggttgggggcaaggtcatagatcaacagcatcccaaggcagaagaatttttcttagtacagaacaaaatggagtctcctatgtctacttctttctacacagacacagcaacaatctgatttctgtatcttttccccacatttcccccttttctactcgacaaaaccgccaacgtcatcatggcctgttctcaatgagctgctgggtacacctcccagacggggtggcagccgggcagaggggctcctcacttcccagaaggggcggccgggcagaggcgccccccacctcccagacggggcggcggccgggcgggggctgccccccacctcccggatggggtggctgcccagcggagacgctcctcacttcccagacggggcggctgctgggcggaggggctcctcacttctccgacggggcggctgctgggcggaggggctcctcacttctcagacggggcagctgccaggcaaaggggctcctcacttctcagacggggcggctgccgggcagagggactcctcacttctcagacagggcggccaggcagagatgctcctcacctcccagacagggttgcggccgggcataggctctcctcacatcccagacggggcggcagggcagaggcgctccccacatctcagacaatgggcggccgggcagagccgctcatcacttcctagacgggatggcggccgggaagaggcgctcctcatttcccagactgggcagccgggcagagggggctcctcacatcccagacgatgggcggccaggcagagacgctcctcacttcccagacggggtggcggccgggcagaggctgcaatctcggcactttgggaggccaaggcaggcggctgggaggtggaggttgtaggtagccaagatcacgccactgcactccagcctgggcaactttgagcactgagtgaacgagactccgtctgcaatcccggcacctcgggaggccgaggctggcagatcactcgcggttaggagctggagaccagcccggccaacacagcgaaaccccgtctccaccaaaaaaatatgaaaaccagtcaggcgtggcggcgcacgcctgcaatggcaggcactctgcaggctgaggcaggagaatcaggcagggaggttgcagtgagccgagatggcagcagtacagcccagcttcggctcggcatcagagggagacggtggaaagagagggagagggagaccatggggagagggagacggagagggagagggagaggaacaatcttcttatatggtttgaaggaatgagaattcacactgaaaaataatttttaattttagtttcagatgtcatcttgataggcaaaacttgtctgccaattaactcatttattgctgaaaattaaataaaattggcattgtttttaaaagtaatgcaagaaagcaaaaagagttatgttgataacagaatcctttattctgtacaagttctagttgcttaagcttaaatcaaatcctgctaagtatattttcttttcttaacaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaaggtatggtagtgaatttgatcaatggggaaattacagatcttttaaacgactgaattgtgtgcataattgttattgcatcagcaaagattgttcatttttagcctattttcattggtttgcatatattaaagggaattgtggaaggtcacagagatatttgttgtttttctgaatacagatctagctgagacatttataaaataagtcaaccatttattcaggcctaccagccctgctcctggtattacctcaactgtggctctatctctttacttctcctcagatcaatgaatctttgtagggcctcttcaaggataaattctcattcattcattctttgaaaaaaaaaaaatatatatatatatatatatgaaacccattgtgtgccaggcttaaacataccagttatctaactaccaaattaagaaaaaaattaaataaatgaattaataaattcttaataggtgaaaatgacttagctcttatcaattgcagggttcttgtcccaaagaaatatatctacatagcaaaatttcaggtgtgagttgtaggttggtgactgtaatatttggggcaggatgatttccaggaggcattaagattataccctatatatttctctggtttaagttagtattggaaaaaaagtactagaaaaatgtgaagcctgttttttgtacctgaaatatcaactccactggcagtttcggagttgaaattatttgaatatggtcaaagaaaaatttcaatggatggaattgggcaaggacgactttattcaagcctatcacagcaggggagagagatcagactgaactaaactccactgaaacaaaaggtgggagagttttaagcgcaggggtgagctaatggaaacgtactggagcaccttgttggaaggaagtgggagcagttgtcaatgtgattaggccatctgtgtttgctaattgtcccttattgaaggtaggctcctactctcccacagacactggggaattccttccttccttccatccctccctccctccctccctcccttccttccttccttcttcttttttttttttttgaaggagttttgctcttgttgcccaagctggagtgcaatggcatgatcttggctcactgcaacttccacctcctaagttcaagcgattctctagcctcagcctcctgagtagctgggattacaggcgtgcaccaccaaacctggctaattttttacatttttagtagaaacgggatttcaccatgttagccagactgatctcaatctcctgacctcaggtgatctgcccaccgcagcctcccaaagtgctgggattacaagtgtgagccaccacgccaggcctctgtcttgataattacatttcaaaggaatggctcccaggtccttggaaaagacattcttggggtataaaactgggaagagtctgggaaaaggggcagagaaagaatttataattccaagtcttctaaagtaaatactctaagaaaagggaggttaggaatttatagttgagaagtctatctaaagtttaataaagtggaggagaacattaaggccattttagtcaacatacatgttctttttgtaacaatttcaacatttttccttttagcaaacaaaaaaattttcttttggttggaaccgctgatggcaagttagcaatttttgaagataagactgttaaggtaaatgttgaatgcattctacatctaaatttattttaagtcttttgttttatatatatctcacacccctcttatgggattataaactccctgagagcaagaatcataaattatgctgtatttgtattgcttcataaaatcttgaacacagtagatcctctgaaaatacttgctgattgactgtatattttatatgaatgaactaagaataaaatgataaatgacatctgattgataatattgggaatggaaataattcaatttgtacataactgaggcagataattccttataaatatattgtggaaaaaaaacaaaaatatacttaagttttaaatatggcttgccattaactttttcttaagcattgaagaaatcatttaattttcttttcttcagattcctatttagtcattaaagcattcatttctctatccatctattcatctttggttccatctattcactcaacttcctacccgttcattctcctattgccaaaaagcttattatctgatgagagacagggaagtagagtataacccttaggttatttcttttgtaatttttacatgggaaaaagaatagattgaatgtaacaataatatttcgaatatgacctaaatttttttatgtataatatttgtacatatttatggggtacatgtgatatgttgttacatgcatagaatgtgtaatgatcaagtcagggtatttaggatatccatcaccatgagcatttatttctctgtgctgagaacatttcaagtctcctagttattttgaaatgtttttaactgtagtcactttattgtactattgaacattagaacttattcctcctatctaactgtatgtttgtacccgttaaccagcctcccttcatcctccccttctcccacacacccatatcctcccaagcctctggtaactatcattctactctctacctccatgagatcaacttttttagctcccacatatgagtgagtacatgtgatatttgtctttctgtgcttggcttatttcacttaacataatgacctccagttccatccatgttgctgtatatgacatgatttcattcctttttatggtcaaatagtattccgttatgtaaatacacacattttctttatgcattcattcattcatgggtgcttaggttgattccacttttttttttagctattgtgaatagtgctgcgataaacatggggatataggaatccctttgatatactgattccctttcctttagattagtatcagtagtaacattgttggattgtatggtagttctatttttaatttttttgagaaatcaccattttgttttccgtagtggctatagcaatttacatacacaccaatagcatatgggcattcgtttttttccgcatccttgccagcatgttattttttgtcttttttataatagccattctaattgggtgaagaagatttcattgtggttttcatttgcatttttactgatgattagttaatgttgagcattttttttcatatatccattggccattactatgtcttcttttgcaaatgtctatttagatcctttgccaactttttgttttgttttaagacagggtcttgctttcttacccaggctggctcacagtggcatgatcatagcttgttgcagccttgaccttctgcactcaagtgatcctccaacatcagcttcacgagtagctgggactacaggcgtgtgctaccatacctggctgtttattttttgtagagatgcggctccactatgttgtccagactgatctcaaactcctgggctcaagcaatcctcctgcctcatcttctcaaagtgctgggattacaggcatgagccaccatacccagccctttgcctacttttaaatggagttcttttttttttttcctgttgaattgctttttcgagtttcttgtgtattctggatgaatagtttgcaaatatttcctcacatttaatggatcctctctatattgttgatagtttcctttgctgtgcagaagctttttagtttattatagtcccatttgtctaattttgtttttgttgcctgtgcttttgggatcttaaccataaactctttgtctagaccaatgttctgaaatgtttcccctgtttccttttaatagtttcatagcttctggtcttacatttaagtctttaatccatcttgagttgaattttgtaaatggtgagagagtggggcctactttcatccttctgcatactgatatccagcttttccagcacaatttattgaaggtggtattctttctcccatgtatgcttttggtgcctttgttgaaaattagttggctttaaatatgtgggtttatttctgggtcctctacattggtctacctacctgtgtttttgccaatactgtgctgttttggttactctagccttgtaatatattttcaagtcaggtagtgtgatgcctccagctttgttctttttgctcaggattgttttggctattttggctctttttcggttccactcaagttttagaaatttttttttctatttctgtgagaaatatcattggagctttcatgggaatttcattgaatctgtagattgctttgggtagtatggtcattttaacaatattaattcttccagtctgtgagcatgaatatctttccatttgtttgtgtcctcttcaatttctttcatgtttttcaggtttccttatagagatcgttcatcatctttgttaaatttactcctaggtattttattaattttttgtagctacttcaaatgggattgctttcttgatttttcagctactttgttgttgttgttgtatagatatgctactgatttctgtatgttgattttgaatcctttactatactcatttatcagaactaagagttttttttaatggaatctttaggtttttgagttttaattttaatatctctaatcatttaagatggaaagtagttttttgaaagcacagattttatggagttttgttctgtggatactcaatttgctgagtgtgttttcttttttttttggcaaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgtaagttattttttatctgtacaagtaatttatcattatacttttgttttttccttataatcattaataatactgttgataattcataaggaagatcttttaaaatgcataatttattttctatcataaaattaaactttcattataaaaaattttgaaaattccagaaagcagaagggtatttttaagaagtcactcaacctaatcacttttagggataaaatatgtaaactcattgtaatcttagtagtatttatcaatctaaattttttaacaatttttattatctgtgtttcaaattagacatgaaattggaagacaatcaactttgtatttcaccaaattcacggactatacatatgcaatttgggtaacttccattaagtattgattgtaggaaagatagacagcaagtattcttgcttgtccaaagttgtttctagatttgataattatacagatgtctactcacagccaagttagtgataccagtttcaaacaagaataaaataaaatattaatataaacctctttcaagtttgctttttttcagtggtattttaatcaaatctttgcagttggttcttatttatcacattcctcagtgataagcagtataggattgtggataagagcataaatcatagttcagatattgctttgccatctattggttttgtgaacttgggcaataacctttcacatcttcagtcatctcttacctgaggattgtaatattctctatctcaaagagatatttggaggagtaaataagaatgttaatatatggatcttaattaacataatgaccagcacctggtaagctgtcaataaacattagctattattattattattagctttgagtcacaaatccctaccttagggaatatcctggtttcccattatccatcaaatttcccaagattggcacttgggagtaatctttgactcctttgttttttgctccctttatccacttgaccctcctaattgtcatttgaatctttgtacttctcactgttctcagtgctggtaccatgggccagactgccatccattatctgtggccacattaacaagagcatccagttctcaccctctgattatactctcttcaacatattttcagcattgcagacagagggacctttctaaaatgtacatcaatctgatgcaattttcctgcttaaaacccttcagtggtatcccattgtcctttatatgaagtccaaattccttaagacagcctactgggcccttcataattcagtgcttgcttacctgtctagattcatattttgcaagctttttgccatctgtgctttacccaaactgaaattgactcagatctctaagacaacctgttattacctttcacccccaacctttgaatgtggtcttctccttacctggatgactattatgccccctgcctatttcagtccaagcaccacttgctctgagtggcaggttaggtgactttgtgcttccattgcatctaatgttttccttcacagtagctataattgtatttgttaaagtagtttctcaataccactaaatctactggctttcaacattggctgtgcatttagaaaccactaggtagctgaaaataatatgatacctgggccctacctcagaccaattaaatcagaccagttaagcctgggatggggatcagattttttttttcaggttctcaagtgattctaaagtatatttgaggttaagatatactgcggagtgcagtgtattataagttcccatgaatgaggatttttatttctgtctttacatatttatttactagtatgtggttaacatttggatcaactcattctcattctgtaatacccacattttaaaaaatgaatgtaaaaatgtcttttattatttttatttttcaaatttaattttagattccaggaatacatgtgcaggtttgttacaaaagtatagtgcgtgatgctgaggtttggaatacaactgaactcacaacccagaaagtgggcatagtgcttgataggtagtttttcatctttgctccccttcctgtaatttccacttttaacccaattatacaagcctgaaaacctttaaaaagaaagggcctccagtttatttttttatttcatagcacattttatgggattatggatatcagttaactctttaaagttccatataagatttggagcatagatgctttactagagagcatccataaagatcaagctctcaaagatgctcatctcccaaaagagattgggacctagatgcataaacacttaaatataaatatttgctctcttctgaacaagtatctcctgtggccttggtctctaccccacaaaacagacatcacatcactaatcaggggttgcctcatcatcagtaccctcatcatcatcagtacacaccgactgagaggcatgttgggtaatgaaagatgactgcctttgaagccggaagactccataggacttttggagttctaggtctgcaacatgtttttagtcctaggtcggcaagcatgatgttagtcataggaccttgagcaaattatttagcatttgtacatgtttctttctactattaaaaacattgagatttatcatattttatgtttttttattaaggatcaagcaagataacacacaaaagtattttataaaatacggaaattccatgcaaaactttgtcctaattggaactattttctattaaatacagcaaatatccaagaaggaattacctaaagcgtagtggctctctgacaacacatcatatttttacctccttttccagaatagaaagaaaagggggaggaaaaaaaatcttcctactctaggatatactaatgattgttaaatctttatggtattttcatgttatctatctgatttaaatgcaattttgactatttttacatattcctcgttgttcattcatactgtagtgccttcttctattcccccacttaacagactgcttgattatatcagaggtgcttatctgtgcaactgtttactggacgaggggtatgtagaaaatacattgtcctcatccttatgaaattacactcataatctagtgtgagggatgcactaataaagacaattttatatttaataaggtctataatatgacagtgacaccagtggggaaaagggactggttggtctattttgataggtcagggaagaaatccagaggagccgacatttaagttcatcctcaaaggccaagtaggagtttgccatgctgatgtggcccaaggtagccaatctgtttgtgaaatatgtacaatgccagatgtcttaggttgaaagggaaatattttaaggtgttcgtaatttttctttatgtttaaaaggggaaaatggcaaatattttactttctgtttatgtttggatgatgtggatttttgttttctataatttgactggcttaactgcaaagatatcccttgctttaaaatttgaagacactgcaactaaattttatttcagcattttatattttataactctaggtataaaaggctaacacttaattttctgagcattcatgaaacaaagttttgcaagaacattcaaaagttacagatataatatttccttcagaaatttagatatagtacaaaattctacaaagagccacatagaattgaaactaaaagtaagaccaaagtaaacattggacataatctttattttattatcacaagaaattaatataaagtaaccaaaagtaagtaaagtaccaaagcatgttatatattcaattcagaatggttagggaagaatatgaaataattgcaatagtctagcttgtttagttttcaaaatagtgtttttacattaagaactaatataaggttgtattacacgtagaaattttaagaagaaaacaaatagtgatgactttctatttttttttctctgtaggttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaaggtaaattctgtggtttttaattttattcccaaaagaattatctttgcacttcatgtgtcacagaggaaggatttttcttcctttctgcctctgaatagagaatttttttaaaatgcagaaaaaaatttgtaatgcttctcagcaccatcttttcagatcaagaaaattttgtcttcagaacataaaagaataggcacataatgtgcatagttttctcatggtattacaaagaatgttctcgaatgaaaatactacattattgaaaatgagcatattggagtctctgctagctttgacatagttctgtcacagtgtcaaatatactatttataattaaattatgggccccaggattatctgctctaaagaaaaagagtcacaaaataatagacaaatatggggggaaatgcaatggactgaccgaggcgctaaggagtggggatcaagaccccagaatgagagcatagtgcttagtctgatgcagcctgtgagtgacaaatccatagcaagcacattctttctgtgctggtgctgagaaacaggaccattttcaagcttatttgctagccactttatatttttattttgttttgattttaccatatagatctatgatactcttgagaacattttagattacacactatatctgtaaaaggatacttcaaagtttcctgtcttagattcatctgacagtttttctatggattgtgagaagggctcacagtttatgttcagaagggccaacaggtctccttgataaagggttccttacttcctgaagtaccaaaacgattaggattcttttatttctggacacttcatttttgtcatgaattagactattcactggttctgggaaaaaattcagtggtttgtatcgatatcttttacatgtgaatgactataattttatgttcctttgtaacattgagacttcatgtaaaacttttgactctaacttttttttttctttatcctgggcacatgtatgctatttttactgaattagatagctttggtatttataaaaattgtatccctcttattcataatttcctgaaaatgaagggctattgttatctttgataatttatgcttcaagtaaagaagtgtggctctttggcatctgtatttagcaaaatttgctttgtataattttaatgatgcataatggtggtggtgtcatgttttaataatttaaaatgttgtttatgttatcatatgtaaatagcatttatctcttaattggtggtaaaattattaatgtatactttatggttctagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggcaagtttctttcctttagatatttttcatattctctaagtcttataaaatatgcctttattttacgtttacattttctctgaactttccagtgtcatatggatggtcttggagggtcacacagtgaaacataagactggtataaattgtgaatagggtcattacagaagtggagggagtaaatgctctcagtcccacaagagaagcagattactgcagctgaacactcagtttgggtcttacttgcttttttcctttttacctaaggcaaaaatgggaaatacatggtattgaatatattttactttttgagcaaagaaaataaagaaaatgtttgttttaatcatagtctagcctcccagcttgttaaagaatctcatttggtttttcattctataacaaatcttttttcttgcagcaatacatgctgaactgcacaacctacaaatattgacaaatcatattttactcaaactttgtctttttttgcttctatttttatatttaaatatgataaaattgtgatagcacataaaatatattttctgcataaatatatttgcgtcttcctttgataataatttgttttagaaaataacaataatagcatatatacaaaagtttacaaaaacgacactatggggtttaattctgaaaaaaactagaatttatgtaactttagcaaataatgaatgttttgaacatggtgaagaaaatatattcattgcaagtatatgtgaaagaggaacatgtgtttttctagcaccttcacctatttttcatttatagactttagagttgcacaggagttacaattagatgctcttaatgactgtaaactattaagatacatgtccacacaagcagagcagtaggtctctcaataggttgtctcagtagtcttattctaccaaagttgttgcattctctagttgaattgtatgtactttgggacccaaatagcttgcttataactgaagttatagtggaatgtctatgggttatagtttgattttaaaataaagatcaattggaggatagcctacaaggtgctgcatgagctggcttcactgtacctctcctgcctccatcatctaccacattcctaccagatcttgcttgtctagatgaacatccagttcttctcaattactatgctatattttgcctcagggatttgcacatgctgtttatttctttgcttagttaacatagctttttttcatgcttatttaactcatatgctttgaaatgttagctcctgtgtcaaaacctctgagaagccaacactgattaggtcaaagttcccgctttgggttcccataacagctttcttgcatagttttgatcatggtcatatttttttttcattaatgctagtctcttcactagaatataaactccaagacggttgggttagtgtgtttttgtttaccccttttttcccaggatctagcctagggcctacatagaagacttttgatgcaaatttgttgaataaattagtgaatgatttgaaaagaaaatatgatattttgacatagtatcagtatatccatccatctaagtgtccatctaaatactcatatttgtactaaatactcatatttgtaccttaccatatccaaagaacttttcacacacattacctcgtttaacattgtaactttgggaagggtaatgtataaatactgagcctattttatagaaaggttaagctgttttctcagactcacataattaagaattgcagcaaggagtggatcacagattttgttattttttaaaaaaatgctggtctttattcaatataattgaagggtcacctagaaaatagaattgtgaattcagttccaaggtatttgtgtcttaaactatgaacaactttacttttttttcaggccagtttaatatatagttttaacagaaaacttacatattttgtttttgtaaaggaagccttaaaaatgtcatgctggtattgggctacaaccggaaaaatactgaaggtacacaaaagcagaaaggtaacatttagaaggatactgttttccaaacagggcaatgatgtgaatgatggtaacatattatgtgtttcataaatttgtagaaaatattacatatggtataatcaggaattttaattggtagtttatagtgtaaagaacttagacataaattttcaaaattacaagtgatatgaagtgttaaatatttatattttcagctgaagtagaggtgtcaatcactagctcaaccttaaacgaaatgtgaatattttttacaacttatctatatctacataatgtctaattttgaacagtgtttgaaaaagcttttatttcttttagaatatgaaatgttaatttattaaatgttgatactctatttgaaatttaatagtttctataatgtattataaaacttttccaagtatagttttttataaataataatttagtacattagttatagctgtgtttatatttacatttatctaagtcaactaaaaatacatgagccaaactgaaataaaataagaatgttttatgatggatctttgaaacatgatttcatttttttctttttctagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaaa

SEQ ID NO: 10 - DNA, 9239 bp, Homo sapiens
gcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaaaaaaaaaaaaaaaaa

SEQ ID NO: 11 - protein, 2527 aa, Homo sapiens
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 12 - DNA, 29556 bp, Homo sapiens
gctagagagggcggggcttggaggggccgcagcgtcacatgaccgcgggaggctacgcgcggggcgggtgctgcttgctgcaggctctggggagtcgccatggtgagtgctgagggggcagtggcacctgggtcgaccctccttgtagcccctgctctctcccaccgccccgcactccagcgagtggagaaggggccccacagaccgttcgggattaagaccagcccgatttggcctgcgggataggggacagcaggaggaaggccgcgggcaggctgatccgggccggggtgggcggcggctcttggctgcggccgttgctgagagacggggcggcctctctgtggggttgacttggcatgtaggctttggggtccatgaaggcctgcggcctcctttaagtggaatcggtcacctgcctaccacgaggggaccggtagtcctaggtctgagcgtctggcccccggggcgcgtggaggccctgagactcggaggtggcgccgggacccgcccagatgttgcgtttctacctttgtgcctagttgtgctcggccgtccccacgccctcctggaggggtcgcagtgattccttggcctttcttggcctcatacccgccttcggctgcagtgtttgtcagcgagttctggggacctgcttacatgaatttcctggaaggactcaggctgtcttctaatcctgacggtcgcaaaggagactgattgtttactttagcatttgtgcattgggcgcaccttgcctcttttgtctcgccattgataaaatccaagtatttgacttgctggaagcagtacttctccttagggcccgtctatgacggcagcaaatcgtggtgtggctgttggccggtaaacttgaacttcctcaaaatgtgaatctttgtgtctggttcccacaaaggcaagttgtcacttgcattttattagcgtttaacatagcctgcactgtgtaaataaattttttgagtatatactgtatgtccgctttaattaccttactcactctgtgtagataggcttctgtaaatctgtaagcctggaaacagatttcactttaaatgtcttaatgccagaaaggattaagtgttttacaaatactattttcatataacgtgttgccgtacaaggtgattttgcctgtttctcaggatttttataattgggaattgatacaagaccggcgcaaaatttaactttaggatttgtgtgtttccagcgtttatggattgacatttatattgttttgtaatggaaaacacttaattgaggatgtattacacactccgattctttgttgggtgaaccagttgggagcaatcagccagacacacagtcttgtcctcatgaattttattgggaaaggaaacttgagtattcgttctttcacagtcactgtctataatttaggcagctcgcttagtcttcttggtgtcgcacttttgcatccgaaagcaagtgttggcggcccctttggcccttcccagccctaaaattccagaatccacttcaattcatcaaacatcttgtgtgccttcagtgtcacggactgtggccctgggtaccaatgtcgaacttaagaggctactgggggagacacgcttgtagaacttaactgtaatacagtgtgtggattgctttactaacattgtcaactcagtgctttggggtcatggagggatagttttgcctggggattttgggaaggactacaggaagtaacgtgaactgagactataaagagggtggaaagaaattctccaggtggaaagaaaaggtattgctaatagatgaagccaaaacgttcagtgtgctccatcagaggattcttggggatatgtattggaaatgaagttagagccgtttggtgttcaaattatattttgaagttgtcatgccctcatcatccttatttctctctacttcagttgtcaatgtatgccatgtcatcatttttaaaatagttctttcattaacgtgatattctgtacctttgtttgaagaaatgagcgttacatttggtttacattgtactgtcagattacatctacagcatataagcagggaggttgaaccacaataggtttctaacataagtggagataattatttgtatctaaactgaataataattatagcacttattaagtgcttattgctaagtagtaggcacgttttgagctttggtttcatttgttcttcataacagtggtgtactctcatctctgtttcatagaagaggaaagaaaagcacattgtttcagtggagagtagttttccaagtgatcagttcatgatctttgctttgctctgcacattagaactgtagacttgagagggctgtttgtgggtctcaagctaaaatgggactaatcacaaatgttaatttgattcccattctgtagggtttccattttcttctgattcatgtagctgtgaagtacggtcatttacaaatggaaaaccactttcatgattgaggaaacattggttaattggcttgtgtttaatgatagcgtgctcatattataactgttaaggctctatgtgacacttcatagtaaggcatcaagaatagcccttataatagctgttgctagcataactacactgttttatgagtaatatataaaaatagattgctttactatagctttatgtcttcacttgtagcttactttgtaataagtcatattctgtaatcttcaacattttgtatttacatttctattttaagctgagttgagacaaaatagaagaattttcctaaaattgcattcttttgtttacacatctctaaatctttctagtcattctatacaaatgtttttggaactgattgtatcctctttgtagccacagtttcatttgctatataataaaaattatcatacacaacagccaaaaggtggaaacaacccatatgtccatggataaacaaaatgtagtgtgtgtgtatatatatagatatataatgaaatacttcgtttttaaaaggagggaaattctggcacatactgcaacatggatgaacattgaagatattatggtcagtgaaataagcgagacataaaagtaaaaatattgtatgatttcacttaaatgaagtatatagagtagtcaaactcagagaaaagaaggtagaattgcctgggggttcggaatgggagtttaatggctacagtttctatttgcatttgtaaagataaaaagttttagagatagatggtggtaattgtattaatgtgaatgtacctaatgccactgaactgtacatttaaatgttaaaatggtaaattttaggtacattttaccacaacaagaagtcatttttaattaaaaatatccagatgtatcataaagaaaaataaaaaaaattcagatgtatcactgtttatctctaaatggatcaattgaacttaatgaaatccattgattcaaattattattaaatctattgcgtccagaggtaaggagccaaaaaattccaaatgatggcctggtttcactaaagttcagagaagactagcccatgatgaatagtaaatttcattaagtcagagtctttaaatgctggtgtcatccttgcctctgaaaccagcattttatggtaatagttccactgggttaaattcatgttccctttaagtgaagtttaaaagatacctaacttcttctttgaaatttgtttgtgcttctgaggaagagtgcttgcagcagagctcagtttactagagtttttcatagggaaaaaaagggagatgcatggtgttgttcattattcagttaatatttttctctttccaaagttagaacaagagaaagttttcaatttttataagctatgctagttcagaagtgggttttatgttatcaagtttctttgttaatctcaaaatgaaatgttgttttgcttttcttagataatgaaacagaccagattttacttgcaggttgatgtgtaagtccttgccttccacctcttcaactcattgtgtgagaggcattttgtctttagtcattgttttaaaaaataaaagtgaaatgacataacaaaaaattaaccattttaaagtgaacaatttataaagtggcatttagtatgctcacaatactgtacaatcaccacctttggtttcaaaacattttccctacccctaaaagaaagccagtgcccattaagtgattattcccctcagcctctctcctggtaatcatcagtctactttctgttcctatacagaatatttcatattagaggaatcatacaatatgtggctttttatgtctagcttctttcatttagcatgatgttttctaggtttgtttatgtagcaatacttcattccttttcatggttgaataatattctgtaattgtatgtatataccacaacttgtttattcatccattaattattcagtctttttttgttaaatatctaaacattctaaaaccagtgtattcatttatgcagtgaacatttgtggagcatattatgtatcaagcagtgtgttggatcccaaggatgtaaaaatgaccgtcatttataataataatgtgatacatgctgtggtggagatgtaaacagtgtttatatgacttgaagaagtggttaattctttttggcatggagattgaattcattttgcaagcagatttttgctgatggcaaagagaagagtgtctagatatattttgtccactgtagctgtagatcagtatatctgaaatggtgtaagctatttgaggacagtgctctttattgttttctactgtaagtcactaacaattttggctgttttatttctagactgtttagtcttttgttaaatattgccaaggaggggctggtcactgttctcgtaaactagttccttagtctgtcttaagaatagactgaaatgcagatgataagtagtctagaggaaaagagaggctttagagattggtttcggctatacctatcacaagatttcgattggtcagatggctatgtctgggttggattcagagtgtgttagcagaacacagccatgaactaccactgcaagtttctttgaggccagcctactttctgagagagaggcaattctttgtacacatactattctcctttgtcagtcttattctgttaacttcagcgataaggcatgactctgtgtgcagcagctgttaataattggtaaatgggctgggtgcggtggctcatgcctataatccaagcactttgggaggtcgaggcaggcgaatcacgaggtcaggagatggagactatcctggctaacatggtgaaaccccgtctctactaaaaatagaaaaaaaattagccgggtgtggtgatgggcgcttgtagtcccagctacttaggaggctgaggcaggagaatggcttgaacccgggaggcggagcttgccatgagccgagatggcaccactgcactccagcctgggcgacagagctagactccgtctcaaaaaaaaaatttggtaaatgggtttgaggtatagagctggacattgttggagaaggactatggctagaactatagaaataacgtacttgctagaagaatgtgcttgagacatcagtggaattttttatttttcagcctacaacacagcagtcccctcaggatgagcaggaaaagctcttggatgaagccatacaggctgtgaaggtccagtcattccaaatgaagagatgcctggtaagaatggagatgtgggaggcacagttgcagttcgtgtgttcctaaggaagcatgtgcagtgtcttctagagtcaggtgtttctggtaaatctaatcttcaccgtttaccagcatctatcttcagtctcatctccctcaagcactttgtggagcaatttcaacaaagagccctgtttactcacatgtatatttatggtttgggattgtctgtcttccctactagaatacaagctcataagaataagagacccttccttttatttacacattactgtattattaccacaccagtgtctgaccagaattactagcctccttggttctatacctcagacctgaggaatatttaacatataataggtactcagtaaatatttgttgaatgaatggatttaaatgctttgcatttgaattattcagctttttttctaaatatcttgaaaactttaatttctttgctgaatagatatatttattgtagaagctagcttaaaaattatacttaacacttatttacatatttttatattctaaaagataaagtaagagataatctgtgtagatacttttgattctctggattaaaatgtaaggaattgagccaaattggttagtactttaaactataaattactgtgatgaagatgatgctattttacctttgtaaaatgtcttactgtgctttctaaagcatagtaatatgctcttgtgtcttttattggtttaattcctaacaaattgggaatgaaaaataaatgtcttggaatggagaagctgggtttgctattgcttgcttctttctcttcctgtgtatggatagtgtttcctctatctcaaggaattgcttgcatttctgagttaagtggaacatatgggcattgtgagggcttgaagaatgcaagaggaaagcaaacttacatggatagtcatttcagacagctctgaagagtctttaacccatgacaaagccatgtcaggatagtatcttccttcacctgaatcagtatgccagttctcttgattgcaggtaaaatgtgatgaatggagctagtttcctagtctctatagattgaaaagattagcattctatcaagaagcttgcagtcttagctatgttaagtcttactaagaatcatgtatctttttctttttcagtagagacggcaaggtgaaccgatctaagttgtttttttaatgtggttaaaatcatttaagtgcggtattcttttaaaactatgtaacaagtccttgatgtaaagaatttgtacaaccaagataaatgtttatttaaattaagcattctcatctattctcttggtatttctgtaggacaaaaacaagcttatggatgctctaaaacatgcttctaatatgcttggtgaactccggacttctatgttatcaccaaagagttactatgaactttgtatcttttgaatgttgaagactaaacatttggaccatacctttttcttgataaggcctattttgtttgttctttatgaagtttttctggagttatcttattcttcgttatctgagtcacatggcactccttctccatgcagatgtgctaagtgagaaaaacactttgagagtactcctttcctatgcttaaacatctttaaatgtgttgtcggtgcatctcaattttcagacccttcatgaggatatttaggctatgacacagttggttctttaatacttagattttgttatgcagcagtctcaaatggacaggaatttaatcatttgccatttcaaaacccattagcagtctgacaggtaaccattgtatttactgctttgcttgaccacacatgctttaaaacccttattttaaagtaagaaaagtccggctaaaattcatccttcgcttgaacactttcttaaaggactaaaacttaagatgtctgcccagtagttagtaatgactccaacaagtttcaaagttttgtttaggttggcttatttttatttttagtccttaatcataattaaaagatatggccatttctgatgaactgcactacttggaggtctacctgacagatgagtttgctaaaggaaggaaagtggcagatctctacgaacttgtacagtatgctggaaacattatcccaaggctgtaagtaattacaaatcagagaacttttgtgtctgtatttctcactatatgttacgtcttttatgattatcagcttaagaaaaagttttaagggtaacttcttaacaaattgagatgaacattttggtagatattctcttacttgttttagagtaactagatttacgttttatgtagatatttgaggaattttggaaatagaaaaaatggacatgcttgctattttttttaatgtcttgactattagaaaaattaatataattgttctcttcctaatatgtttaaaggtaatatctatgttgtatatatacagtgtgtgtgtgtgtgtgtgtgtgtgtgtgtgtatagtttttttagaggtcagtcagtggttatattttaaatgagatattttccttgtcatgcgggagaaaacaacatggttcctgtcttgtttatttaatgttttgttcagtgtgtttggaaataaattcttgatttgaatattttatttctaatcagcatttcttcataattttcctagttaccttttgatcacagttggagttgtatatgtcaagtcatttcctcagtccaggaaggatattttgaaagatttggtagaaatgtgccgtggtgtgcaacatcccttgaggggtctgtttcttcgaaattaccttcttcagtgtaccagaaatatcttacctgatgaaggagagccaacagagtaagtgattttctttcttaattttgttgcaatatttctttcattgtagaatgtataaaagtgtggaaacatatacagaaacaaagtgtgaataattcttccacccagtcagccatttaggtagcatttgtatatagatttcctttgtaatatagaactcctcagtatatgtggtatcatctaaaatgtactcttatgcaaattttatctttggattgttaggacctgcttttttcatttaatgtaattttttctactacattaaatcttctttgaaaataaaacttttttaagagagttgtatttggaaattgaatttgtaatgaaataataaagtgtgagccagctggatttcataattgttcctttagtgtctatcagtttttataatttatagactgctagttaccttggaatataagtgatttgaattatctgttacgagttagctattaactccagagaaggaaaaataaaagccattcagagacactcctgtctcttgtgttatcagtattctagcatcaaagtctactgtacttttatcccacagcaggggcagatggtcagccaactgtggtcttcagtggggtgagctgttcacatgacaggtccccagattaaagaacttcattccttttttaaaaagtttattcatttattttctttctttttttaatttttaatcttttttcagtttgccccaacagattttgtttttttctttttaatattttcatttatttctaaggtttttaatatatcatttatttctaatgttttttaatatatctgcatcaatttcttttaaaacagtacagaaaagataaaacatttaacaatgtagagaaattgatgaagttacttgctttattatgttttgagtgtccgttttgagcatttaattaggcaatcaataacaatttttgaaaggtactgaggtctccatcctaggagacgtagaaaaataaagcaggaaatccatggtctcttccctcacaaagcttacattccaattaaaaacaaaatattcaacagtaaaatgatgtagttagcagtacaccataagtgttacattttttagccttttgtttttgtttttggtttgtggggatggggtctcattatgttgcccaggccagttttaaactcctggcctaaagcgatcttcctgccttggcctcccaaagcactggattacggacatgagccaccatgcccatccttctagcctttttcaattaaggaagttgccataagagcaagtccagttggcccaggatgaggagttggggaaagtaatttgccctttaaatttatactgtcctctccatggtactttttaccctagagtctgttccctttgaaatttaacactaagccaatgaagttgaaagtgattttttataaagcattggtgtactatagagataagtaggaaatacacaaaggagaaggatagtagtaagttggtcctgtaaatactgtgtaaagacttttctgtttctttgcagtgaagaaacaactggtgacatcagtgattccatggattttgtactgctcaactttgcagaaatgaacaagctctgggtgcgaatgcagcatcagggacatagccgagatagagaaaaaagagaacgagaaagacaagaactgagaattttagtgggaacaaatttggtgcgcctcagtcagttggaaggtgtaaatgtggaacgttacaaacaggtttatatatttttgttacctcttcttatgttcagagataaactgaaatctgatttttaaaatcagaatatttttgttatacaatagtacattgaaaaacatcttaaaatggctgttattgaagaagacttaaacggaaagatatatatgcagtgtttgtggattggaagacttaatattgtcaaaatagcatttaaaaagaattgatttatagatccaatgtaatctcagtcaaaatcccagcagacttttgtagaaattaagaagctgattctaaagtttatatgaagaaacaaagaacctggaacagctacaacgaatttgaaaaggaagaacaagttgaaagacccaagcaacctgaattaatgatttactctaaagctgcagtgagatcctgtgtggtattggtgaaaaggatagacacacaaatcaatggaggggaataaaacagggaatggcaaactttacctgtgagggaccagataataaatggtttttggctttgtaagacatgtgctgtacaacaagcttgtccaacctgcagcccaggacagccttgaatatggcccaacataagtttgtaaactttaaaacatgagattttttgctttttttttttttttttttttttttttttaaagctcatcagctaagtgtattttatgtatggcccaagacaattctaattcttcttcaagccaaaagattggacaccctagtctacaactaataacagtgcagatatggtgcaaaagcacccacaggcaatatggaagtgaatgggcatggctgtgttctagtaaaactttatttgtaaaaacaagcagcagctcagtttaccgatctctgactggacaatccataatagacccagatatttatgatcagttatttttgataaaagtacaaaggcaaccttttcagcaagtgattctggaacaattggatgtttatatgcaaacaaaaaaccctgaaccttgacccatccctcataccatatagaaaaaacacagaaatcaatcagagacctaaatatagaacctaataatgttagaagaaaacacagaggaaatctttatgacctaggattagacaaagatttctgaggatatacaagcacaagccatgaagaaaaaagctcacttttgagaggccaaggcagatggatcacttgagtccaggagtttgagacaggcctgggcaacatagggagaccccatctctacaaaaattaccaaaattagctgggcatggtggaacgtacctgtagtcccagcactcaggaggcttgaggtgggaggatgacttgagcctaggaggtggatgttgcattgagtggagattgtgccacttcactccagcctgggcaaccgaacaagaccttgtctcaaaaagaaaaaagcttttaaagtttagaagtgaagtcttggtgagaaaaatctcaaatacgattttcaagttagtagttcaaatgcgttactagaggaatagcttaagattttgaaaacagattttaacccttatgtgtgttttttctcttttagattgttttgactggcatattggagcaagttgtaaactgtagggatgctttggctcaagaatatctcatggagtgtattattcaggtagctgggaacatttcatttttttttaaacgacctattttatctttcattaaatttaattgttttgaaaaaattttgatggaataggaaataagctttcctgaataaagagttttccttgcggggtgtggtgactcacacctgtaatctcagcagtttgggagttcaaggtgggaggatctcttgaggccaggagttcaaaaccagcctgggcaacatagcacgatgccgtttctataaaaaattaaaaaaatttttttagtgtttcttttttttttcatgtaatcttgcttcttctaaaaataatttaaaaataggaattttctgtttctaacttataccttggtctttgtatcaatgtggtttgttttcctccaaaatgtaggaatgagtaatctgagttttctaggtctctgtagctttagtttaattgtaggtgcactttgtttattggaatatttctgtctgagcttatgtttagtagagaggttcaaaagtaatgtgtttgaatttagttgtataagaatacagtgtttttttcccacaaatgtgaactttaccatatgtgagtccagaatattacgtgaaatacttttatttgtattgatcatttgattttcaggttttccctgatgaatttcacctccagactttgaatccttttcttcgggcctgtgctgagttacaccagaatgtaaatgtgaagaacataatcattgctttaattgataggtaagaccttccaacactggcggataaatgctctgacttgggaataatgaattttaaacatttttttgaattatttgtttctgttacatctttatcataccaatgatcttaatttaattatactataaataatttagctttgtgagtatgagtactaggtacttgtctaggttagacatgaaagaggcttaacttaaatgtgcaggagacgtgaagataatgaatatctttattctgtgtgcttaattgacatttaaagatgttgtacagacttattttttaaatcatacaaatccaaagatcatattgaagaacaaaatttgttttttaccatgatgtaagtatcttgcagtgggaactcatttgatttagagtagccgtaagatactgatgattgaaaatgttcaagtaatcactctatcatcacattttcttaaagaaaaaattttaagtatcaaatatgtttagtacatccacttttttattttcttaggtttttttttttttttttttttttgagacagatcctcactcttgtcacccaagctagagtgcagtgacgctgtctcggctcactgcaacctctgactcctaggttcaagtgattctagtgtctcagcctccggagtagctgggattacagacatgcaccaacaagcccagctaatttttgtatttttagtagagacagggttttgccaggttggctaggctagtctcaaactcctgagctcaaatgatctgcctgcctcagcttcccaaagtgctgggattacagacatgagccactgcgcttggccaatgggtggcttttttgcagccatgttatgtagtagtatatgatgtctgtcctacacttgtaagcattgtcatgaaaccagaaacctaagagaagatttatttctgcagataccttttgtatgttttttaaaaaactaagttattagttttaaagtctgagaatttagataacaaatttttccaaattgtcagctcaatcctgggcagcaaaaattccatacttattgggcccactcttaaaggaagctagtaactggattttcctgagttgcctgtaatgtcacttacacatctctgtcagtagtgatgcttctgggcatagcaaaatgtggatgtagttgtgactgacaaacagataatgataatgaaacatactattttgagtaatttaagatgtgggaaataaaagttaattttatgaattttagacttagttgtatttcaagctttagtaaaaatgcagtatcttaaaatagtctatgtacttttattttttaaaggttatttatttaaatcatggttgttgaatacatttgtcactttaatgcatttctgtccatatctgcttaattatgcttcaaagagttgagagaattatcttgttgaaaatctacttaatatggtgtgaaataagaatgctgatgaaaaaggtttcattggcaaaactgtttagttaaaaatgaattgaggaggccgggtgcagtggctcacatctgtattcccagcactttgggaggccaaggagggaggcttgcttgagtccaggtcagtaccaccctgggcaacatggtgaaaccccatcactacagaaaacacaaaaattagctgggtatggtggcacatgctgttagccccagctactcaggaggctgaggtggaaggaggatagcctgagctcagcaggtggaggtttcattgagtggagagtgcgtgactgcactccagcctgggcgacagagcgagactctgtctcaaaacaaaacaaaacaaaacaaaaaaaacaaaaaccttttgggctcatacaaaatatagaaaagcaataaagaataagatgtcatccatgatctcactacccaaaccctgtatcttttaaaataaaggggtgttttttttttttttagattagctttatttgctcaccgtgaagatggacctggaatcccagcggatattaaactttttgatatattttcacagcaggtggctacagtgatacaggtttgtgtagcatttctcctaagttctcaaaactttgaaacttctctgccttccttttacaattgtttaaaataaattgtgtggttttctaaacattccagtctagacaagacatgccttcagaggatgttgtatctttacaagtctctctgattaatcttgccatgaaatgttaccctgatcgtgtggactatgttgataaagttctagaaacaacagtggagatattcaataagctcaaccttgaacagtaagtcagttacatttttgtaaaaatcctcaaagatatttttgtcctagatttgcttttctttctcaattgttttttgaactgctggcatttgtcttgttttaatcatgcattaagattgtcatgcttagcactactaggggcagaaagtagtgaccaattacttgtttttttatattaaggaaattgtggtacctatggaccataggcagtcttcagggaccagtgtctccaatttggatccctttctgtgtgtcaggggcatccaatcttttggcttccctgggctgcactggaagaagcattttcttgggccacacataaaatacactaacactaacaatagctgatgagcttaaaaaaaaaatcccaaaaaaactcataatgttttaagaaagtttacgaatttgtgttgggccgcattcaaagccatcctgggctgcatgcggcctgtgggctttgggttggacaagcttgcatgtgactgagtttgttcttaaactggtaaggaaactttgtcaggcagtatttatttccataagtggtgttttcctacgaatcagcacatggtgaaaaatgaggggctatgtatatttaaggtgcagaattaaattggtttaaatatcttttctattttgagctttgattttgataccttaaaggaaatatcaacagtactatttccaacctgaagcctcctcagctgttctgtcctagacttatggcgtcctctagtggccactatgggcagctatgatcctgttaccttccccagcagttcccttcctgccctgttccccactgctctggcttgggtcaagccaggcctgcctcccgccaacatattcttcagaattttacctcatgtaatcttcctcctttctatctcccttccagtggtttacctgcatcaagaaaatttcttctttttttcctccctttgtgttacccttgttcttttggtcatttttggttttgtgtgtgtgcaaactgaaaacaagtccagatgtggaatgataagtgtgagagaaaattaaatgatgtgccaggtgtggtggcttgcacctgtaatcccagctattcaggaggctgaattgggagaatcacttgagtccatgagtttgagaacagcctgggcaacatagcgagaccccgtctctaataaaaaataaaattaaaaataaaaaaaatttaaattaaaaaaactaaatgatgtatctgtgtctttctccccaagtgaattttaaagtaaaaatagacaaagtaattagaaataacaacctctaaagaggttgtaataaatgccccaatatgcctcaatatctacagaatgattttactaacaactacgtaaaagtcagtcagcctgcttttccttaatcaccaacatctgatgcagaagaaatagtttatgtgtttttctgttgtgtcaaattgctggttttgcatggagtttttttcctatttattttcatcatgaatatacaatacttgttggctggcccctgggaaccaaactaccacttaaaatacttcccttagaaatgtcatcaaattctagacagtcatcttaactccagctataccatctgttcatgagttggaaactgtatctagttttgtatcaacagaaaaataatagatgaatatatatttgtgtttagataagcatttttatcctcctgaaaggaggttgttatagtcttctgtggtggtatgattcacttgacccatttcctttaatgtgtaatgaaaaatttcaaattcttatggaacaaatgctatttgtgtatatagaaagttaattttattcattaagacttctgtttttctttttgtagtattgctaccagtagtgcagtttcaaaggaactcaccagacttttgaaaataccagttgacacttacaacaatattttaacagtcttgaaattaaaacattttcacccactctttgagtactttgactacgagtccagaaagagcatgagttgttatgtgcttagtaatgttctggattataacacagaaattgtctctcaagaccaggtaagagaatacctacgtgctattttagggaaacagtgttacaattttagactttggacctagatacctgagatgggaggggagggtaattcaatactaaataaaatttacaagtaactttttcattatataaattaaaaattggagatgtataaagaattataaaacatttataattccaccagatagagaataaccactgttaattaacatttggtgcatatctttccagacttttgtctgtatatgtgtgtatgacatacatgtgtatcgactttctcaccaaaaaaaggaatatcttgttgatactgtattgtaattttataactggaaacacttttgataatggctttgtatgccaatggtttcacctcagtgggtttcttgtgcctcgcatgttacaggtggattccataatgaatttggtatccacgttgattcaagatcagccagatcaacctgtagaagaccctgatccagaagattttgctgatgagcagagccttgtgggccgcttcattcatctgctgcgctctgaggaccctgaccagcagtacttggtatgagtttacccttagtatatccctgtatcagctcctagtgaaatcacatgttcaagtgcttaaaatggtttaattcactttctggtcttagatggttttgaaggaattgcaactgaattaaagattcacttgaacctgggaggcggaggttgtagtgagccgagattgtgccactgcactccagcctgggcaacacagcgagactccatctcgaaaaaaaaaaagattcatggcatccatgggcttttactttatatataaacacataattgtttgtaaacttctggagcatgtgagtaacaattcagttgctctgatttcttttgaagactctctgagaattacaaaaaagtctgtcttcttttgcttgagtgccgataattattccatgttcattttttctgaactatgtattgcttataataaactttataagaaatacaattcttatatttaattttacttttccaaatttgcaagtataaattatatttgtcatattgaaaatgtgagtttttgttttttgatgaaagatttaaaaattcattttgcctttttcttaacttttttttttctgataaagaacaatcacatgaggttctctctttattattagtccacagggaatcattgtgaaatggataaaacatgttgcctgagtaggtgtatcagtgaccgatactagatagatagtttattttagtgaagggttagcacagttggctgcttaattattgtttgggcaaagtagtttaaccattcttggatgcataaggctattaggctgctatgatgaaaaagacatttgcttgaggatgtcctgactgtctcatccctttctgttgactttcttcattgtagttgacacacctgtacttcataatcagtgtgaaataagaggctgacttctgttgatagtgtgatggtctttgtcttggtttagtgacaaacattccaggactgtggtattgtgctctgtgagctatgtgatctgtacagagtgactgtcttaagtattttaactgattgccttatgtttctgtgtgagattgtttgtatctgtgtgttttcattttctattgcctaccaaatatagtagttagaaactattccttccggcggggcatggtggctcgcatctataatcctggcactttgagaggctgaggtggatggatcacctgaggtcaggagttcaggatcagcatggccaacatggtgaaaccccatctctactaaaaatgcaaaaaattagctgggcgtggtggtgggcgcctgtaatcctagctgctcaggagactgaggcaggagaatcacttgaacctgggaggtggaggttgcagtgagtggatatcatgccattgcactccagcctgggcaacaagagtgaaactccgtctcaaaaaaaattatgccttctgcatgtggctgattggttattcccatgtatggagatctttaatgatagggtcattagctctgactgcccctaggggaaatgcattctcttattcatctaccatatcaggaatttcacaaaacctgaatgccattgtgtcacatatactaaaaatattttataaactctgtgtttttcttgtaatttttctgaattggctatatgttgtgccatttcagaaaaaaaaatccaagaaaaacacagaattcatggaatatttcacaagtagctcttttaaagtatgttagcattttccttgacttaaatggtcttaaaatttttttgaatgaggaggtatgatgtaccagtaatatgcatatagttgttgtgtatcatagtaatagttaatattactgagcttatgccttgtgctaagtagtggtaagccttcacatgtgtcacttgatcttcccaacaaccctaggagtttatagaaacttgtggctaagagaaggtaaataatttgcccaaggccacacatgtaataagtattagcatctgcttttaaatgtgagtctctgagtatcttcacagccttctttttttctcttttcttttttctttttttttttttttgagatggaatcttgctctgtcacccaggctggagtgcagtggcatgatcccagcttactgcaacctccatctcctgggttcaagcaattctcctgcctcagcctcctcagtagctgagattacgggtgtgcgccaccatgcccagctaatttttgtatttttagtagatacagggtttccctatgttggccaggctggtctcgaactcctgacctcaactgatctgtccaccttcggcctcccaaagtgctgggattacagacatgagtcaccacacctggccagagcctacattctttatcagtgcagcatactttgcacatgtgtgtatgaaaatatatttaaatatatctttgcttctaactcgctaccttgggcaggttatacaacctctctgaaactcaggcttccccatttgtgaaatggaatagtatctgtctctgggttgttgtgacaacttgaggagataagaaatatgtaaattgcctaccataaagtatggtacattgtatatattcacaaaatgttagcaatgatgattagagcccacatttatttcacaaatgattaatcagagtttggaaatttttttttctttaatgcttttgggtcagattttgaacacagcacgaaaacattttggagctggtggaaatcagcggattcgcttcacactgccacctttggtatttgcagcttaccagctggcttttcgatataaagagaattctaaagtggtgagtttacttttaagtatttaggtactttttttcctctttcatcactctgagtgtgtgtgtgtttgttttatattataaaaaatttcaaacgtacaaaaatagacagtggtataataaaatcccattttcgccaactctctatttgttacttatcctgtgctaagtgttcctaacggtgatggtggtggatcacatactgagggatcacgtaaaaagcacttagaaatgcaagattaaagcagtgtgaatttatgctgaaactctttcctaagttctaattcaggttagctttaaaacctaaggagagggcctagcattgcagtcgtttctctctaaaggcatatcattgaataatatgagttgtgggcaactttttatgagcttttttcttcctcaaaatggaaccatggcttgagtcttcacagtgtagttttgaagaaaatacctcaagctcacgtacctgaaagttggacattcaggttaatgttaaggaacaacctcagtaacttaattttgtttgtttgtttgttttgagatagggtctcattctgtcgcccgggctggagtacagtggcgcagtcttagctcactgcaacctccaactcctgggttcaagcgattcttgtgtgtcagcctcctaagtagctgggattacaggtgtgcaccaccatgcccagctaatttttgtatttttagtagagacagggtcttgccatgttgaccagttggtctcgaacttgtggcctcaggtgatcctgctgcctcagcctctcaaagtgctaggattgtaggtgtgaaccactgcatctggcctcagtatggacttgattttctcgtaatagagaaaaaagatgtatgcagtagacctaccagcatgaaacagcagcttttggccaatttttattaggccagcttatcattcactctttaccagcgtttatggataggaatttgtgaatataacaataaaaatagcaaccagcctacattacaaagccatagtaattaaagcagtatggtaatatggtactggcataaaaacagacacatagaccaatggaacagaatagagagtctagaaataaacccacacatatgcaataaactaatctttgataaggacaccaagaatacacaaaggggaaaagaatggtctcttcaataaatagtattgggaaagttggatatccacatgcaaaagaacgcatttggactctcatcttatgccatatataataatgaactcaaaatggattaaagacctgaaaccataaagctcctagaagaaaacatagggaaaaacctccttgtcattggtcaatgattttttggatatgaaaccaaaaacctatgcaactaaagcaaaaataagtttaaaaataagcaaaaaataagtttaaaataagcttaaaataagcaaaaataagtttaaaataagcaaaaaataagcaaaaataagtttaataaactaaaaaccttctgtacaacaaaggaaacaatcagcagagtgaagagacaggcaatggaatgggggagaatatttgcaaactatacatctgaaaagtggtcaatatctaaaatatatatggaatgcaactcaatagcaagcaaatgaataacttgatttaaaaatgagcaaaggatctgaatagacatttttccaaagaagacatacaggtggccaactggtatatgaacagatgttcaacatcatttatcaggaaaatgtaaatcaaaaccactatgagatgtcacctcacatctgtcagaataactgttatcaaaaaaacagaaaatcaagtgttggcaaggatgtagagaaatgggaaccctgtttattattggtgggaatataaattagtatagccattatggaaaacagtatggaggttcctcaaaaaactgaaactagaactaccatgtgaccctgcagtcccacatctagttatgcattcaaaggaaaggaaatcagtatctcaaagagatatctgcactcccatgtttattgcagcattattcacaatggctgagatatggaaacaaccttagtgtccatcgatagatgagtaaagaaattgtgttgtgtatatatgtgtgtgtatatacgtatatatgtgtgtatatgtatgtacgcacatattctctacatagtagaataatactcagctatagaaatgaagaaaatcttgccatttatgacaacagggattaatctggaggacattgttctaagtgaaataagccaaacacagaaaggcaaatactatatgacttcatttatatgtagaattgttttttaagttgaattcatccagcctgggtaatatagcaagacccaatctctattaaaaaataaaaaggccaggtgtgggggctcacgcctgtaatcgcagcactttgggaggccgaagcaggcggttcacctgaggtcgggagtttgagaacagcctgaccaacatggagaaaccccgtctctactgaaaatacaaaattagctgggcgtggtggcgcatgcctgtagtcccagctactcgggagtctgaggcaggagaatcacttgaacccgggacgcagaggttgtggtgagccgagatcgtgccattgcacttcagcctgggcaacaagagtgaaactccgtctaaaaaaaaaaaaaaaaaattaaaaaattagccaggcgtggtggtacatgcctatagtcctagctactcaagaggctagggcagggctgggtgtggtggctcacacctgtcatcctagcactttgggaggccaaggtgggtggatcatttgggatcaggagtttgagaccagcctggccaacatggtgaaaccctgtctctactaaaaatacaaaaattagccaggtgtgagggcacatgcctgtaatcccagctacttgggaagctgaggcaggagaatcacttgaacccgggaggcagaggttgcagtaagctgagatcgcgccactgcactccagcctgggtgacaaagtgagactctgtctcaaaaaaaagaggctagggcaggaggacagcttgagcccaggagttggaggctgcagtgagttatgattgtgcttttgcgctccagcctgggtgacagagggagacccagtcactaaaaaatggttgaacttgtgtaagcaaagactagaacagtagttgccaaagaatacaaactttcagttataagataaaaaaattctggggatcaaaacgatttagggcaaataaataaaagtaactagcctttacttatttactagcatttcttactgtgttgtcacccactgtgccaaggtctatgactgccactgtcacttttttttttttttttgagtcaaggtctttgttgcccagactgggatacagtggtatgattacggctcactgcagcttcgaactcttaggctcaagcgatccttccatttcagcctcctgagtagctgggactgcaagcatgtgccaccacactggctaattttttattttttgtagagacagagtctcaccatgttgcctaggctggtctgaaactcttgggctcaagcgatcctcctgcctccttggcctccctaagtgttgggattacaggcatgagccaccatgcccagcctgtcgccatcttttaaaaatgaaaagaactgattgctttaacaagaagaaatttggatagtcaatcatgataaaatatttaacctcgcttgtaattacaacagcgaaccttttaagaaatcaaattggcaaagagaaatgaaaaataaggttcagcaatggcgatggtgtgatgaaaattcattctcatctaatgttggcagtgtgaattactataatacttctaggaagttggcggtgtgtaagtagggtgttaaaatattcaataattttacttccaagtggaattccaagaatttatactaagggaataattagggtctcaataaagcttagtgtatatagaacattcattgtaatattacagattatgtctaaaagggaatagttcaataaattatgccatagccagtctccataatattttctagtcattaaaatgatttcgaattagtatcgggaagattgttaggacaaaataggaaaaattagagctgggtgcagtggctcacgcctgtaatcctagcactttgggaggctgaggcaggcggatcacctgaggtcgggagtttgagaccagcctgaccaatatggagaaaccccgtctctactaaaaatacaaaattagctgggtgtggtggcgcatgcctgtaatcccagctattcggtaggctgaggcaggagaatcacttgaacctgggaggcggaggttgtggcgagctgagatcatgccattgcactccagcctgggcaacaagagggaaactacttctcaaaaaaaaaaaaagaaaagaaaagaaaaattagatacaaattacttgaagtgtgaatcgattttaactctcaagaaaataaggtctagatgcagtggctcacgccagtaatcctagcactttgggaggctgagatgggtggatcacttgaggtcagcagttcaagactagcctggccgataaggtgaaaccccttctctactaaaaatacaaaaaatagcagggcgtggtggcgcgcttgtaatcccagctactcaggaggctgaggcagaagaatggtttgaacccaagaggcagaggtggcagtgagccgagatcgcaccaaagagaaaaaagaaaaccacacacaaaaatgccagttatattacagttacatagaaaaaaagaaaggaagacatttagcatccgaatgttaccagtgattatccgtgggtggtagatttagggatgatgtgtggatgattttgtgtatttttctaattttctccaatttgggaatgtaacttacaaatcagaaaaaacaattatcagccaggtgtgatggctcatgcctgtaatcccaacactttgggaggctgaggcgggaggtttgctcggggccagtagttcaagatcagcctgggcaacagaatgagaccctgtctctacaaaaaaaaaaaaaaaaaaaaaaaaaaaaattagccaggtgtggtgatgcaagcctgtagtcctagctattcgggagtctgaggtgggagcatcacttgaacccaggagttcaaggctgcaatgagctgtgatcacaccactgcactccagctgggtaacagagctgttgaaaaaaaaaaaaggaaagaaaaaacaggttgagtgcagtggctcacgcctgtgatcccagcactttgggaggctgaggcgggcagatcacttgaggtcaggagttaccagcctggccaacttggtgaaaccccgccccacttggtgaaaccccgcccctactaaaaatacaaaattagctgggtgtggtggtgggcacctgtaatcccagctactcgggaggctgaggcaggagaatcacttgaatccaggagacggaggttgcagtgagccaagattgtgccactgcactccagcctgggcaacaagagcaaaactctgtctcaaaaagaaaaaacaaataccaaatacattaacattgcaaaggcaatttaacctcaaatgatgttttgagaagacatcctgcttgatttacttgtttgccctataactgaaacagagaaggaaaatgacaggaaaactgtgcacacaacttacagtattttgttctattaaaatggatatcctggaacaagttaattttgaatttaaggtaacttaaaatgttttttcttgttttaggatgacaaatgggaaaagaaatgccagaagattttttcatttgcccaccagactatcagtgctttgatcaaagcagagctggcagaattgcccttaagactttttcttcaaggagcactagctgctggggaaattggttttgaaaatcatgagacagtcgcatatgaattcatgtcccaggtgatgatctgttctttctgcgttgtcatgtcagctctgctgggttcagttgcttgtttgcaggcatggtggtaatgcacatgaatttactcttcttttactgaatgtgtaactaccaccttcccaccatcatggaacctgttaatattattgttgtaattgactggtgttgatcatttgctgatgaaatctaagatttccaagtgggtcatggtaaaaatgtttcatggaacataaaattcgggaaatgcactcaattcccaaaatccagtttgggaaccctgggttaaacaaagttgaaagaagtttctttattgcaactttttagcgtttttaccatctcagttgtgtcctgtggctctcaagagagggtgcagcatgttctgatatgaaggctgcagaagtctcacaggatggaggtttggtgacaagtactttggaaaatgctcaactagaggatggttggtccttgaaagtcctttctgctttatgttcactaggcattttctctgtatgaagatgaaatcagcgattccaaagcacagctagctgccatcaccttgatcattggcacttttgaaaggatgaagtgcttcagtgaagagaatcatgaacctctgaggactcagtgtgcccttgctgcatccaaacttctaaagaaacctgatcagggccgagctgtgagcacctgtgcacatctcttctggtctggcagaaacacggacaaaaatggggaggaggtaaggtcattcctgactgcatgatagcagacaggatccataacagggatcagttgtcatggccttgtgttctggaggtgaaacatttggggtgcttggaaatctgatgaacaaaattgctttgttttgttaaaaaagagagtctcatcctgtagtgaagcctctgctttgaggatattgtaacatagcaagttcaaaccactacctgtttttaaaaaaatacagctgtatacttcaaaacaagaagaaggagaatgaaaaggatttaaatttgttatgtccctttaaaacacgaaagagccacggtagtgttgtgtttctttgtatgaaaacgagatgtttcattaatctcttcactgtccccctgcccttttattttagcttcacggaggcaagagggtaatggagtgcctaaaaaaagctctaaaaatagcaaatcagtgcatggacccctctctacaagtgcagctttttatagaaattctgaacagatatatctatttttatgaaaaggaaaatgatgcggtaagtgaattagtaaagtgttgttaataaactaatattttcccttcctactcttaggagatttgatatgtacaaaagtttatcattctgatactttaatcactgttcatttgaaaaatgtaaaataatttacagatgtcaaataataggctaatttgtcataatgttctagtttaagataattcctaggctgggcgtggtggctcatgcctgtaatcccagcactttgggaggctgaggcaggcagatcacctgaggtcaggagtttgaaaccagcctggccaacattgtgaaaccccatctctactaaaaatacaaaaattagctaggcgtggtggcaggcgcctgtaatcccagctacttggaagcctaaggcaggagaatcgcttgaacctgggaggtggaggctgcagtgagccaagactgtgccattgcactcctgcctgggcaacaagagtgaaactccgtctcaaaaataataataataataattcctaaacgcagtatccttttagcaatacagttttggtcaagatttgtaagttaaataaaattttgcttgtttttctttttttgacagagtctggctctgtcacccaggctggagtgcagtggcaatctcagctcattgcaacctctgcctcccaggttcaagcaattctcatgcctcagcctcctgaatagctggtattataggcgcccggcaccacgcccagctaatttttgtattattactagagatggggttccaccatgttggccaggctggtctcaaaactcctgacttcaagtgatctgcccacctcagcctcccaaagtgctgggagtacagggatgagccactgagccgagccaattttgcttgttttaaagggttgtttttttttttttttttttgatagtcagtaattgttcaaactaggaattgtatccccatctttcttttttcataattactcaggtaattgatgagtgtaacagaagctcctcaaaacagttttattaaattgcctttcattttttgtggtacgtgcttgatcatgaatttgtacatattcttttgtaggtaacaattcaggttttaaaccagcttatccaaaagattcgagaagacctcccgaatcttgaatccagtgaagaaacagagcagattaacaaacattttcataacacactggagcatttgcgcttgcggcgggaatcaccagaatccgaggggccaatttatgaaggtctcatcctttaaaaaggaaatagctcaccatactcctttccatgtacatccagtgagggttttattacgctaggtttcccttccatagattgtgcctttcagaaatgctgaggtaggtttcccatttcttacctgtgatgtgttttacccagcacctccggacactcaccttcaggaccttaataaaattattcacttggtaagtgttcaagtctttctgatcaccccaagtagcatgactgatctgcaatttaaaattcctgtgatctgtaaaaaaaaaaaaaaaaaaaaaaacaaaacccacaagcacttatcttggctactaatgaagctctccttttttttgtttgtttgtttgcttcattgttgattgtgtattttcttcattcctggggagtactaacccaaaagcgtctgtctcttgttttctagtccagtttgagattaatttagaagaaaggaatactgtatgtgaaattcatcttgggctttcccctaaattgcaagataaggccatgtgtaagattttccctaaaactagaatatattaatgcatgtttgagaattttaaagcaccatggtcaaaaccagaagctatattttgcatatttggactcagccatccattaagaacccatgttgtcctctggacatatttatcaatataattgggttttaaatagtataaaagaaaacttgtgatctatataatttatgtatcaccttcattgtaaatttagcaggaaatgcatcacaattatgatttttttttttgcaccagtgaaacaataaagatgctattaacaa

SEQ ID NO: 13 - DNA, 3298 bp, Homo sapiens
gctagagagggcggggcttggaggggccgcagcgtcacatgaccgcgggaggctacgcgcggggcgggtgctgcttgctgcaggctctggggagtcgccatgcctacaacacagcagtcccctcaggatgagcaggaaaagctcttggatgaagccatacaggctgtgaaggtccagtcattccaaatgaagagatgcctggacaaaaacaagcttatggatgctctaaaacatgcttctaatatgcttggtgaactccggacttctatgttatcaccaaagagttactatgaactttatatggccatttctgatgaactgcactacttggaggtctacctgacagatgagtttgctaaaggaaggaaagtggcagatctctacgaacttgtacagtatgctggaaacattatcccaaggctttaccttttgatcacagttggagttgtatatgtcaagtcatttcctcagtccaggaaggatattttgaaagatttggtagaaatgtgccgtggtgtgcaacatcccttgaggggtctgtttcttcgaaattaccttcttcagtgtaccagaaatatcttacctgatgaaggagagccaacagatgaagaaacaactggtgacatcagtgattccatggattttgtactgctcaactttgcagaaatgaacaagctctgggtgcgaatgcagcatcagggacatagccgagatagagaaaaaagagaacgagaaagacaagaactgagaattttagtgggaacaaatttggtgcgcctcagtcagttggaaggtgtaaatgtggaacgttacaaacagattgttttgactggcatattggagcaagttgtaaactgtagggatgctttggctcaagaatatctcatggagtgtattattcaggttttccctgatgaatttcacctccagactttgaatccttttcttcgggcctgtgctgagttacaccagaatgtaaatgtgaagaacataatcattgctttaattgatagattagctttatttgctcaccgtgaagatggacctggaatcccagcggatattaaactttttgatatattttcacagcaggtggctacagtgatacagtctagacaagacatgccttcagaggatgttgtatctttacaagtctctctgattaatcttgccatgaaatgttaccctgatcgtgtggactatgttgataaagttctagaaacaacagtggagatattcaataagctcaaccttgaacatattgctaccagtagtgcagtttcaaaggaactcaccagacttttgaaaataccagttgacacttacaacaatattttaacagtcttgaaattaaaacattttcacccactctttgagtactttgactacgagtccagaaagagcatgagttgttatgtgcttagtaatgttctggattataacacagaaattgtctctcaagaccaggtggattccataatgaatttggtatccacgttgattcaagatcagccagatcaacctgtagaagaccctgatccagaagattttgctgatgagcagagccttgtgggccgcttcattcatctgctgcgctctgaggaccctgaccagcagtacttgattttgaacacagcacgaaaacattttggagctggtggaaatcagcggattcgcttcacactgccacctttggtatttgcagcttaccagctggcttttcgatataaagagaattctaaagtggatgacaaatgggaaaagaaatgccagaagattttttcatttgcccaccagactatcagtgctttgatcaaagcagagctggcagaattgcccttaagactttttcttcaaggagcactagctgctggggaaattggttttgaaaatcatgagacagtcgcatatgaattcatgtcccaggcattttctctgtatgaagatgaaatcagcgattccaaagcacagctagctgccatcaccttgatcattggcacttttgaaaggatgaagtgcttcagtgaagagaatcatgaacctctgaggactcagtgtgcccttgctgcatccaaacttctaaagaaacctgatcagggccgagctgtgagcacctgtgcacatctcttctggtctggcagaaacacggacaaaaatggggaggagcttcacggaggcaagagggtaatggagtgcctaaaaaaagctctaaaaatagcaaatcagtgcatggacccctctctacaagtgcagctttttatagaaattctgaacagatatatctatttttatgaaaaggaaaatgatgcggtaacaattcaggttttaaaccagcttatccaaaagattcgagaagacctcccgaatcttgaatccagtgaagaaacagagcagattaacaaacattttcataacacactggagcatttgcgcttgcggcgggaatcaccagaatccgaggggccaatttatgaaggtctcatcctttaaaaaggaaatagctcaccatactcctttccatgtacatccagtgagggttttattacgctaggtttcccttccatagattgtgcctttcagaaatgctgaggtaggtttcccatttcttacctgtgatgtgttttacccagcacctccggacactcaccttcaggaccttaataaaattattcacttggtaagtgttcaagtctttctgatcaccccaagtagcatgactgatctgcaatttaaaattcctgtgatctgtaaaaaaaaaaaaaaaaaaaaaaacaaaacccacaagcacttatcttggctactaatgaagctctccttttttttgtttgtttgtttgcttcattgttgattgtgtattttcttcattcctggggagtactaacccaaaagcgtctgtctcttgttttctagtccagtttgagattaatttagaagaaaggaatactgtatgtgaaattcatcttgggctttcccctaaattgcaagataaggccatgtgtaagattttccctaaaactagaatatattaatgcatgtttgagaattttaaagcaccatggtcaaaaccagaagctatattttgcatatttggactcagccatccattaagaacccatgttgtcctctggacatatttatcaatataattgggttttaaatagtataaaagaaaacttgtgatctatataatttatgtatcaccttcattgtaaatttagcaggaaatgcatcacaattatgatttttttttttgcaccagtgaaacaataaagatgctattaacaaaaaaaaaaaaaaa

SEQ ID NO: 14 - protein, 796 aa, Homo sapiens
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL

SEQ ID NO: 15 - DNA, 22 bp, Artificial Sequence
  Description of Artificial Sequence Synthetic primer
cagcaaacactacaagtccacg

SEQ ID NO: 16 - DNA, 22 bp, Artificial Sequence
  Description of Artificial Sequence Synthetic primer
cagctgaagccgcactatctcg

SEQ ID NO: 17 - DNA, 22 bp, Artificial Sequence
  Description of Artificial Sequence Synthetic primer
gacagcaagctcacactaccca

SEQ ID NO: 18 - DNA, 22 bp, Artificial Sequence
  Description of Artificial Sequence Synthetic primer
tctgtccaacctgtgaaaccgt

SEQ ID NO: 19 - DNA, 22 bp, Artificial Sequence
  Description of Artificial Sequence Synthetic primer
ggagggcgtctagggaatcgag

SEQ ID NO: 20 - DNA, 22 bp, Artificial Sequence
  Description of Artificial Sequence Synthetic primer
cttcagggtcagcttgccgtag

SEQ ID NO: 21 - DNA, 17 bp, Artificial Sequence
  Description of Artificial Sequence Synthetic primer
tccgatctgannnnnnn

SEQ ID NO: 22 - DNA, 60 bp, Artificial Sequence
  Description of Artificial Sequence Synthetic primer
aatgatacggcgaccaccgagatctacactctttccctacacgacgctcttccgatctct

SEQ ID NO: 23 - DNA, 63 bp, Artificial Sequence
  Description of Artificial Sequence Synthetic primer
caagcagaagacggcatacgagatcggtctcggcattcctgctgaaccgctcttccgatctga

SEQ ID NO: 24 - DNA, 18 bp, Artificial Sequence
  Description of Artificial Sequence Synthetic primer
ggtgagcctccgcactcg

SEQ ID NO: 25 - DNA, 18 bp, Artificial Sequence
  Description of Artificial Sequence Synthetic primer
ttcccacccaccgcctgt

SEQ ID NO: 26 - protein, 203 aa, Homo sapiens
MGSRDHLFKVLVVGDAAVGKTSLVQRYSQDSFSKHYKSTVGVDFALKVLQWSDYEIVRLQLWDIAGLERFTSMTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 27 - protein, 2527 aa, Homo sapiens
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYSIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 28 - protein, 2527 aa, Homo sapiens
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKACASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 29 - DNA, 20 bp, Homo sapiens
tcgtcagaacagcccagaca

SEQ ID NO: 30 - DNA, 20 bp, Homo sapiens
tcgtcagagcagcccagaca